SEQUENCE LISTING (324 sequences)

SEQ ID NO: 1 - protein, 18 aa, synthetic construct
  REGION 1..18: note = FV linker 1
GSTSGSGKPGSGEGSTKG

SEQ ID NO: 2 - protein, 15 aa, synthetic construct
  REGION 1..15: note = FV linker 2
GGGGSGGGGSGGGGS

SEQ ID NO: 3 - DNA, 54 bp, synthetic construct
  misc_feature 1..54: note = FV linker 1
  source 1..54: mol_type = other DNA
ggctctacaagcggctctggcaagcctggatctggcgagggaagcaccaagggc

SEQ ID NO: 4 - DNA, 45 bp, synthetic construct
  misc_feature 1..45: note = FV linker 2
  source 1..45: mol_type = other DNA
ggtggaggaggctcaggaggaggtggctctggtggtggaggctcg

SEQ ID NO: 5 - protein, 27 aa, Homo sapiens
FWVLVVVGGVLACYSLLVTVAFIIFWV

SEQ ID NO: 6 - DNA, 81 bp, Homo sapiens
  source 1..81: mol_type = genomic DNA
ttctgggtgctggtggtcgtgggcggagtgctggcctgttatagcctgctcgtgaccgtggccttcatcatcttttgggtg

SEQ ID NO: 7 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - DNA, 321 bp, Homo sapiens
  source 1..321: mol_type = genomic DNA
atcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatct

SEQ ID NO: 9 - protein, 111 aa, Homo sapiens
RVKSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 333 bp, Homo sapiens
  source 1..333: mol_type = genomic DNA
cgagtgaagagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 11 - protein, 41 aa, Homo sapiens
RSKRSRLLHSDYMNMTPRRPGPTRKHYQPYAPPRDFAAYRS

SEQ ID NO: 12 - DNA, 123 bp, Homo sapiens
  source 1..123: mol_type = genomic DNA
cgcagcaagcggagccggctgctgcacagcgactacatgaacatgacccccagacggcccggacccaccagaaagcactaccagccttacgcccctcccagagacttcgccgcctacagatct

SEQ ID NO: 13 - protein, 39 aa, Homo sapiens
IEVMYPPPYLDNEKSNGTIIHVKGKHLCPSPLFPGPSKP

SEQ ID NO: 14 - DNA, 117 bp, Homo sapiens
  source 1..117: mol_type = genomic DNA
atcgaagtgatgtacccccctccctacctggacaacgagaagtccaacggcaccattatccacgtgaagggaaagcacctgtgccccagccctctgttccctggccctagcaagcct

SEQ ID NO: 15 - protein, 239 aa, Homo sapiens
  VARIANT 1: note = X is any amino acid
  VARIANT 20: note = X is any amino acid
XGSTSGSGKPGSGEGSTKGXIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 713 bp, Homo sapiens
  misc_difference 1: note = n is any nucleotide
  misc_difference 56: note = n is any nucleotide
  source 1..713: mol_type = genomic DNA
nggctctacaagcggctctggcaagcctggatctggcgagggaagcaccaagggcnatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 17 - protein, 8 aa, Homo sapiens
GGSISSNY

SEQ ID NO: 18 - protein, 7 aa, Homo sapiens
VSYSGST

SEQ ID NO: 19 - protein, 12 aa, Homo sapiens
ARESYYYYGMDV

SEQ ID NO: 20 - protein, 118 aa, Homo sapiens
QVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 21 - protein, 8 aa, Homo sapiens
GFTFSTYQ

SEQ ID NO: 22 - protein, 8 aa, Homo sapiens
IVSSGGST

SEQ ID NO: 23 - protein, 12 aa, Homo sapiens
AGELLPYYGMDV

SEQ ID NO: 24 - protein, 119 aa, Homo sapiens
EVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 25 - protein, 8 aa, Homo sapiens
GGSISSYY

SEQ ID NO: 26 - protein, 7 aa, Homo sapiens
INHSGST

SEQ ID NO: 27 - protein, 12 aa, Homo sapiens
ARCPIYYYGMDV

SEQ ID NO: 28 - protein, 118 aa, Homo sapiens
QLQLQESGPGLVKPSQTLSLTCTVSGGSISSYYWSWIRQPPGKGLEWIGEINHSGSTNYNPSLKSRVTISVDTSKNQFSLKLSSVTAADTAVYYCARCPIYYYGMDVWGQGTTVTVSS

SEQ ID NO: 29 - protein, 9 aa, Homo sapiens
SRDVGGYNY

SEQ ID NO: 30 - skipped

SEQ ID NO: 31 - protein, 9 aa, Homo sapiens
WSFAGSYYV

SEQ ID NO: 32 - protein, 109 aa, Homo sapiens
QSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVL

SEQ ID NO: 33 - protein, 6 aa, Homo sapiens
QSISSY

SEQ ID NO: 34 - skipped

SEQ ID NO: 35 - protein, 9 aa, Homo sapiens
QQYYSTPQT

SEQ ID NO: 36 - protein, 107 aa, Homo sapiens
DIQMTQSPSAMSASVGDRVTITCRASQSISSYLNWYQQKPGKAPKLLIYAASSLQSGVPSRFSGSGSGTDFTLTISSLQPEDFATYYCQQYYSTPQTFGPGTKVDIK

SEQ ID NO: 37 - protein, 6 aa, Homo sapiens
QSISSY

SEQ ID NO: 38 - skipped

SEQ ID NO: 39 - protein, 9 aa, Homo sapiens
QQYESYRRS

SEQ ID NO: 40 - protein, 107 aa, Homo sapiens
DIQMTQSPSSLSASVGDRVTITCRASQSISSYLNWYQQKAGKAPKLLIYAASSLQSGVPSRFSGSGSGTDFTLTISCLQSEDVATYYCQQYESYRRSFGQGTKVEIK

SEQ ID NO: 41 - protein, 6 aa, Homo sapiens
QSISSY

SEQ ID NO: 42 - skipped

SEQ ID NO: 43 - protein, 9 aa, Homo sapiens
QQYNSYSRT

SEQ ID NO: 44 - protein, 107 aa, Homo sapiens
DIQMTQSPSSLSASVGDRVTITCRASQSISSYLNWYQQKPGKAPKLLIYAASSLQSGVPSRFSGSGSGTDFTLTISCLQSEDFATYYCQQYNSYSRTFGQGTKVEIK

SEQ ID NO: 45 - protein, 107 aa, Homo sapiens
DIQMTQSPSSLSASVGDRVTITCRASQSISSYLNWYQQKPGKAPKLLIYAASRLESGVPSRFSGSGSGTDFTLTISCLQSEDFATYYCQQYNSYSRTFGQGTKVEIK

SEQ ID NO: 46 - protein, 6 aa, Homo sapiens
QSISSY

SEQ ID NO: 47 - skipped

SEQ ID NO: 48 - protein, 10 aa, Homo sapiens
QQYNSYSPCT

SEQ ID NO: 49 - protein, 108 aa, Homo sapiens
DIQMTQSPSSLSASVGDRVTITCRASQSISSYLNWYQQKPGKAPKLLLYAASRLESGVPSRFSGSGSGTEFTLTISSLQPDDFATYYCQQYNSYSPCTFGPGTKVDIK

SEQ ID NO: 50 - protein, 6 aa, Homo sapiens
QDISRY

SEQ ID NO: 51 - skipped

SEQ ID NO: 52 - protein, 8 aa, Homo sapiens
QQYDNLIT

SEQ ID NO: 53 - protein, 106 aa, Homo sapiens
DIQMTQSPSSLSASVGDRVSITCRASQDISRYLNWYQQKPGKAPKLLLYAASRLESGVPSRFSGSGSGTDFTLTITSLQPDDFATYYCQQYDNLITFGQGTRLEIK

SEQ ID NO: 54 - protein, 6 aa, Homo sapiens
QDISRY

SEQ ID NO: 55 - skipped

SEQ ID NO: 56 - protein, 9 aa, Homo sapiens
QQYNSYSRT

SEQ ID NO: 57 - protein, 107 aa, Homo sapiens
DIQMTQSPSSLSASVGDRVSITCRASQDISRYLNWYQQKPGKAPKLLLYAASRLESGVPSRFSGSGSGTDFTLTITSLQPDDFATYYCQQYNSYSRTFGQGTKVEIK

SEQ ID NO: 58 - protein, 6 aa, Homo sapiens
QSISSY

SEQ ID NO: 59 - skipped

SEQ ID NO: 60 - protein, 8 aa, Homo sapiens
QQYDNLIT

SEQ ID NO: 61 - protein, 106 aa, Homo sapiens
DIQMTQSPSSLSASVGDRVTITCRASQSISSYLNWYQQKPGKAPKLLIYAASSLQSGVPSRFSGSGSGTDFTLTITSLQPDDFATYYCQQYDNLITFGQGTRLEIK

SEQ ID NO: 62 - protein, 6 aa, Homo sapiens
QSVSSN

SEQ ID NO: 63 - skipped

SEQ ID NO: 64 - protein, 9 aa, Homo sapiens
QQYNSYSRT

SEQ ID NO: 65 - protein, 107 aa, Homo sapiens
EIVLTQSPATLSVSPGERATLSCRASQSVSSNLAWYQQKPGQAPRLLIYGASTRATGIPARFSGSGSGTEFTLTISRLEPEDFATYYCQQYNSYSRTFGQGTKVEIK

SEQ ID NO: 66 - protein, 6 aa, Homo sapiens
QSISSY

SEQ ID NO: 67 - skipped

SEQ ID NO: 68 - protein, 9 aa, Homo sapiens
QQYESYSRT

SEQ ID NO: 69 - protein, 107 aa, Homo sapiens
DIQMTQSPSSLSASVGDRVTITCRASQSISSYLNWYQQKAGKAPKLLIYAASSLQSGVPSRFSGSGSGTDFTLTISCLQSEDVATYYCQQYESYSRTFGQGTKVEIK

SEQ ID NO: 70 - protein, 9 aa, Homo sapiens
SSDVGGYDF

SEQ ID NO: 71 - skipped

SEQ ID NO: 72 - protein, 9 aa, Homo sapiens
SSYAGSNSV

SEQ ID NO: 73 - protein, 109 aa, Homo sapiens
QSALTQPPSASGSPGQSVTISCTGTSSDVGGYDFVSWYQQHPGEAPKLLVYDVNNRPSGVSNRFSGSKSGNTASLTISGLQAEDEGDYYCSSYAGSNSVFGTGTKVTVL

SEQ ID NO: 74 - protein, 9 aa, Homo sapiens
SSDVGGYEF

SEQ ID NO: 75 - skipped

SEQ ID NO: 76 - protein, 10 aa, Homo sapiens
SSYTSSSTYV

SEQ ID NO: 77 - protein, 110 aa, Homo sapiens
QSALTQPASVSGSPGQSITISCTGTSSDVGGYEFVSWYQQHPGSAPKLIIYDVIERPFGVSYRFSASKSGNTASLTISGLQGEDEADYFCSSYTSSSTYVFGTGTKVTVL

SEQ ID NO: 78 - protein, 9 aa, Homo sapiens
GSDVGAYDY

SEQ ID NO: 79 - skipped

SEQ ID NO: 80 - protein, 10 aa, Homo sapiens
SSYSGSSTWV

SEQ ID NO: 81 - protein, 110 aa, Homo sapiens
QSALTQPASVSGSPGQSITISCTGTGSDVGAYDYVSWYQHHPGRAPRLIIRDVSVRPSGVPDRFSGSKSGNTASLTISGLQAEDEADYYCSSYSGSSTWVFGGGTKLTVL

SEQ ID NO: 82 - protein, 9 aa, Homo sapiens
SSDVGSYNL

SEQ ID NO: 83 - skipped

SEQ ID NO: 84 - protein, 11 aa, Homo sapiens
SSYTSSSTFAV

SEQ ID NO: 85 - protein, 111 aa, Homo sapiens
QSALTQPASVSGSPGQSITISCTGTSSDVGSYNLVSWYQQHPGKAPKLMIYDVSNRPSGVSYRFSGSKSGNTASLTISGLQAEDEADYYCSSYTSSSTFAVFGGGTQLTVL

SEQ ID NO: 86 - protein, 9 aa, Homo sapiens
SSDVGGYNY

SEQ ID NO: 87 - skipped

SEQ ID NO: 88 - protein, 9 aa, Homo sapiens
CSYAGGYYV

SEQ ID NO: 89 - protein, 109 aa, Homo sapiens
QSALPQPASVSGSPGQSVTISCTGTSSDVGGYNYVSWYQQHPGKAPKLMIYDVSKRPSGVPDRFSGSKSGNTASLTVSGLQAEDEADYFCCSYAGGYYVFGTGTKLTVL

SEQ ID NO: 90 - protein, 9 aa, Homo sapiens
SSDVGGYNY

SEQ ID NO: 91 - skipped

SEQ ID NO: 92 - protein, 12 aa, Homo sapiens
SSYAGSGSTPFV

SEQ ID NO: 93 - protein, 112 aa, Homo sapiens
QSALTQPPSASGSPGQSVTISCTGTSSDVGGYNYVSWYQQHPGKAPKLMIYDVSKRPSGVPDRFSGSKSGNTASLTISGLQTEDEADYYCSSYAGSGSTPFVFGTGTKLTVL

SEQ ID NO: 94 - protein, 9 aa, Homo sapiens
SSDVGGYNY

SEQ ID NO: 95 - skipped

SEQ ID NO: 96 - protein, 9 aa, Homo sapiens
CSYAGRRYV

SEQ ID NO: 97 - protein, 109 aa, Homo sapiens
QSALTQPASVSGSPGQSITISCTGTSSDVGGYNYVSWYQQHPGKAPKLMIYDVSNRPSRVPDRFAGSKSGNTASLTISGLQAEDEADYYCCSYAGRRYVFGTGTKLTVL

SEQ ID NO: 98 - protein, 245 aa, synthetic construct
  REGION 1..245: note = Anitigen biding domain
QVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSGSTSGSGKPGSGEGSTKGQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVL

SEQ ID NO: 99 - protein, 243 aa, synthetic construct
  REGION 1..243: note = Anitigen biding domain
DIQMTQSPSAMSASVGDRVTITCRASQSISSYLNWYQQKPGKAPKLLIYAASSLQSGVPSRFSGSGSGTDFTLTISSLQPEDFATYYCQQYYSTPQTFGPGTKVDIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 100 - protein, 243 aa, synthetic construct
  REGION 1..243: note = Anitigen biding domain
DIQMTQSPSSLSASVGDRVTITCRASQSISSYLNWYQQKAGKAPKLLIYAASSLQSGVPSRFSGSGSGTDFTLTISCLQSEDVATYYCQQYESYRRSFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 101 - protein, 243 aa, synthetic construct
  REGION 1..243: note = Anitigen biding domain
DIQMTQSPSSLSASVGDRVTITCRASQSISSYLNWYQQKPGKAPKLLIYAASSLQSGVPSRFSGSGSGTDFTLTISCLQSEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 102 - protein, 243 aa, synthetic construct
  REGION 1..243: note = Anitigen biding domain
DIQMTQSPSSLSASVGDRVTITCRASQSISSYLNWYQQKPGKAPKLLIYAASRLESGVPSRFSGSGSGTDFTLTISCLQSEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 103 - protein, 244 aa, synthetic construct
  REGION 1..244: note = Antigen biding domain
DIQMTQSPSSLSASVGDRVTITCRASQSISSYLNWYQQKPGKAPKLLLYAASRLESGVPSRFSGSGSGTEFTLTISSLQPDDFATYYCQQYNSYSPCTFGPGTKVDIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 104 - protein, 242 aa, synthetic construct
  REGION 1..242: note = Antigen biding domain
DIQMTQSPSSLSASVGDRVSITCRASQDISRYLNWYQQKPGKAPKLLLYAASRLESGVPSRFSGSGSGTDFTLTITSLQPDDFATYYCQQYDNLITFGQGTRLEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 105 - protein, 243 aa, synthetic construct
  REGION 1..243: note = Antigen biding domain
DIQMTQSPSSLSASVGDRVSITCRASQDISRYLNWYQQKPGKAPKLLLYAASRLESGVPSRFSGSGSGTDFTLTITSLQPDDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 106 - protein, 242 aa, synthetic construct
  REGION 1..242: note = Antigen biding domain
DIQMTQSPSSLSASVGDRVTITCRASQSISSYLNWYQQKPGKAPKLLIYAASSLQSGVPSRFSGSGSGTDFTLTITSLQPDDFATYYCQQYDNLITFGQGTRLEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 107 - protein, 243 aa, synthetic construct
  REGION 1..243: note = Antigen biding domain
EIVLTQSPATLSVSPGERATLSCRASQSVSSNLAWYQQKPGQAPRLLIYGASTRATGIPARFSGSGSGTEFTLTISRLEPEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 108 - protein, 243 aa, synthetic construct
  REGION 1..243: note = Antigen biding domain
DIQMTQSPSSLSASVGDRVTITCRASQSISSYLNWYQQKAGKAPKLLIYAASSLQSGVPSRFSGSGSGTDFTLTISCLQSEDVATYYCQQYESYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSS

SEQ ID NO: 109 - protein, 246 aa, synthetic construct
  REGION 1..246: note = Antigen biding domain
QSALTQPPSASGSPGQSVTISCTGTSSDVGGYDFVSWYQQHPGEAPKLLVYDVNNRPSGVSNRFSGSKSGNTASLTISGLQAEDEGDYYCSSYAGSNSVFGTGTKVTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 110 - protein, 246 aa, synthetic construct
  REGION 1..246: note = Antigen binding domain
EVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSGSTSGSGKPGSGEGSTKGQSALTQPPSASGSPGQSVTISCTGTSSDVGGYDFVSWYQQHPGEAPKLLVYDVNNRPSGVSNRFSGSKSGNTASLTISGLQAEDEGDYYCSSYAGSNSVFGTGTKVTVL

SEQ ID NO: 111 - protein, 247 aa, synthetic construct
  REGION 1..247: note = Antigen biding domain
QSALTQPASVSGSPGQSITISCTGTSSDVGGYEFVSWYQQHPGSAPKLIIYDVIERPFGVSYRFSASKSGNTASLTISGLQGEDEADYFCSSYTSSSTYVFGTGTKVTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 112 - protein, 247 aa, synthetic construct
  REGION 1..247: note = Antigen biding domain
QSALTQPASVSGSPGQSITISCTGTGSDVGAYDYVSWYQHHPGRAPRLIIRDVSVRPSGVPDRFSGSKSGNTASLTISGLQAEDEADYYCSSYSGSSTWVFGGGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 113 - protein, 248 aa, synthetic construct
  REGION 1..248: note = Antigen biding domain
QSALTQPASVSGSPGQSITISCTGTSSDVGSYNLVSWYQQHPGKAPKLMIYDVSNRPSGVSYRFSGSKSGNTASLTISGLQAEDEADYYCSSYTSSSTFAVFGGGTQLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 114 - protein, 246 aa, synthetic construct
  REGION 1..246: note = Antigen biding domain
QSALPQPASVSGSPGQSVTISCTGTSSDVGGYNYVSWYQQHPGKAPKLMIYDVSKRPSGVPDRFSGSKSGNTASLTVSGLQAEDEADYFCCSYAGGYYVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 115 - protein, 249 aa, synthetic construct
  REGION 1..249: note = Antigen biding domain
QSALTQPPSASGSPGQSVTISCTGTSSDVGGYNYVSWYQQHPGKAPKLMIYDVSKRPSGVPDRFSGSKSGNTASLTISGLQTEDEADYYCSSYAGSGSTPFVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 116 - protein, 246 aa, synthetic construct
  REGION 1..246: note = Antigen biding domain
QSALTQPASVSGSPGQSITISCTGTSSDVGGYNYVSWYQQHPGKAPKLMIYDVSNRPSRVPDRFAGSKSGNTASLTISGLQAEDEADYYCCSYAGRRYVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 117 - protein, 245 aa, synthetic construct
  REGION 1..245: note = Antigen biding domain
QLQLQESGPGLVKPSQTLSLTCTVSGGSISSYYWSWIRQPPGKGLEWIGEINHSGSTNYNPSLKSRVTISVDTSKNQFSLKLSSVTAADTAVYYCARCPIYYYGMDVWGQGTTVTVSSGSTSGSGKPGSGEGSTKGQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVL

SEQ ID NO: 118 - protein, 467 aa, synthetic construct
  REGION 1..467: note = Chimeric antigen receptor
QVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSGSTSGSGKPGSGEGSTKGQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 465 aa, synthetic construct
  REGION 1..465: note = Chimeric antigen receptor
DIQMTQSPSAMSASVGDRVTITCRASQSISSYLNWYQQKPGKAPKLLIYAASSLQSGVPSRFSGSGSGTDFTLTISSLQPEDFATYYCQQYYSTPQTFGPGTKVDIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 465 aa, synthetic construct
  REGION 1..465: note = Chimeric antigen receptor
DIQMTQSPSSLSASVGDRVTITCRASQSISSYLNWYQQKAGKAPKLLIYAASSLQSGVPSRFSGSGSGTDFTLTISCLQSEDVATYYCQQYESYRRSFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 465 aa, synthetic construct
  REGION 1..465: note = Chimeric antigen receptor
DIQMTQSPSSLSASVGDRVTITCRASQSISSYLNWYQQKPGKAPKLLIYAASSLQSGVPSRFSGSGSGTDFTLTISCLQSEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 465 aa, synthetic construct
  REGION 1..465: note = Chimeric antigen receptor
DIQMTQSPSSLSASVGDRVTITCRASQSISSYLNWYQQKPGKAPKLLIYAASRLESGVPSRFSGSGSGTDFTLTISCLQSEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 466 aa, synthetic construct
  REGION 1..466: note = Chimeric antigen receptor
DIQMTQSPSSLSASVGDRVTITCRASQSISSYLNWYQQKPGKAPKLLLYAASRLESGVPSRFSGSGSGTEFTLTISSLQPDDFATYYCQQYNSYSPCTFGPGTKVDIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 464 aa, synthetic construct
  REGION 1..464: note = Chimeric antigen receptor
DIQMTQSPSSLSASVGDRVSITCRASQDISRYLNWYQQKPGKAPKLLLYAASRLESGVPSRFSGSGSGTDFTLTITSLQPDDFATYYCQQYDNLITFGQGTRLEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 465 aa, synthetic construct
  REGION 1..465: note = Chimeric antigen receptor
DIQMTQSPSSLSASVGDRVSITCRASQDISRYLNWYQQKPGKAPKLLLYAASRLESGVPSRFSGSGSGTDFTLTITSLQPDDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 464 aa, synthetic construct
  REGION 1..464: note = Chimeric antigen receptor
DIQMTQSPSSLSASVGDRVTITCRASQSISSYLNWYQQKPGKAPKLLIYAASSLQSGVPSRFSGSGSGTDFTLTITSLQPDDFATYYCQQYDNLITFGQGTRLEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 465 aa, synthetic construct
  REGION 1..465: note = Chimeric antigen receptor
EIVLTQSPATLSVSPGERATLSCRASQSVSSNLAWYQQKPGQAPRLLIYGASTRATGIPARFSGSGSGTEFTLTISRLEPEDFATYYCQQYNSYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 465 aa, synthetic construct
  REGION 1..465: note = Chimeric antigen receptor
DIQMTQSPSSLSASVGDRVTITCRASQSISSYLNWYQQKAGKAPKLLIYAASSLQSGVPSRFSGSGSGTDFTLTISCLQSEDVATYYCQQYESYSRTFGQGTKVEIKGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 468 aa, synthetic construct
  REGION 1..468: note = Chimeric antigen receptor
QSALTQPPSASGSPGQSVTISCTGTSSDVGGYDFVSWYQQHPGEAPKLLVYDVNNRPSGVSNRFSGSKSGNTASLTISGLQAEDEGDYYCSSYAGSNSVFGTGTKVTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 569 aa, synthetic construct
  REGION 1..569: note = Chimeric antigen receptor
EVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSQSALTQPPSASGSPGQSVTISCTGTSSDVGGYDFVSWYQQHPGEAPKLLVYDVNNRPSGVSNRFSGSKSGNTASLTISGLQAEDEGDYYCSSYAGSNSVFGTGTKVTVLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 469 aa, synthetic construct
  REGION 1..469: note = Chimeric antigen receptor
QSALTQPASVSGSPGQSITISCTGTSSDVGGYEFVSWYQQHPGSAPKLIIYDVIERPFGVSYRFSASKSGNTASLTISGLQGEDEADYFCSSYTSSSTYVFGTGTKVTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 469 aa, synthetic construct
  REGION 1..469: note = Chimeric antigen receptor
QSALTQPASVSGSPGQSITISCTGTGSDVGAYDYVSWYQHHPGRAPRLIIRDVSVRPSGVPDRFSGSKSGNTASLTISGLQAEDEADYYCSSYSGSSTWVFGGGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 470 aa, synthetic construct
  REGION 1..470: note = Chimeric antigen receptor
QSALTQPASVSGSPGQSITISCTGTSSDVGSYNLVSWYQQHPGKAPKLMIYDVSNRPSGVSYRFSGSKSGNTASLTISGLQAEDEADYYCSSYTSSSTFAVFGGGTQLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 468 aa, synthetic construct
  REGION 1..468: note = Chimeric antigen receptor
QSALPQPASVSGSPGQSVTISCTGTSSDVGGYNYVSWYQQHPGKAPKLMIYDVSKRPSGVPDRFSGSKSGNTASLTVSGLQAEDEADYFCCSYAGGYYVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 471 aa, synthetic construct
  REGION 1..471: note = Chimeric antigen receptor
QSALTQPPSASGSPGQSVTISCTGTSSDVGGYNYVSWYQQHPGKAPKLMIYDVSKRPSGVPDRFSGSKSGNTASLTISGLQTEDEADYYCSSYAGSGSTPFVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 468 aa, synthetic construct
  REGION 1..468: note = Chimeric antigen receptor
QSALTQPASVSGSPGQSITISCTGTSSDVGGYNYVSWYQQHPGKAPKLMIYDVSNRPSRVPDRFAGSKSGNTASLTISGLQAEDEADYYCCSYAGRRYVFGTGTKLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 467 aa, synthetic construct
  REGION 1..467: note = Chimeric antigen receptor
QLQLQESGPGLVKPSQTLSLTCTVSGGSISSYYWSWIRQPPGKGLEWIGEINHSGSTNYNPSLKSRVTISVDTSKNQFSLKLSSVTAADTAVYYCARCPIYYYGMDVWGQGTTVTVSSGSTSGSGKPGSGEGSTKGQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - DNA, 1401 bp, synthetic construct
  misc_feature 1..1401: note = nucleotide of Chimeric antigen receptor
  source 1..1401: mol_type = other DNA
caggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcaggctctacaagcggctctggcaagcctggatctggcgagggaagcaccaagggccagagcgagctgacacagcctagatccgtgtctggcagccctggccagagcgtgaccatcagctgtaccggcaccagcagagatgtgggcggctacaactacgtgtcctggtatcagcagcatcccggcaaggcccccaagctgatcatccacgacgtgatcgagcggagcagcggcgtgcccgatagattcagcggcagcaagagcggcaacaccgccagcctgacaatcagcggactgcaggccgaggacgaggccgactactactgttggagcttcgccggcagctactacgtgttcggcaccggcaccgatgtgaccgtgctggcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 139 - DNA, 1395 bp, synthetic construct
  misc_feature 1..1395: note = nucleotide of Chimeric antigen receptor
  source 1..1395: mol_type = other DNA
gacatccagatgacccagtctccatctgccatgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcagcctgaagattttgcaacttactattgtcagcaatattatagtactcctcaaactttcggccctgggaccaaagtggatatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 140 - DNA, 1395 bp, synthetic construct
  misc_feature 1..1395: note = nucleotide of Chimeric antigen receptor
  source 1..1395: mol_type = other DNA
gacatccagatgacccagtctccatcctccctgtctgcatctgtaggagacagagtcaccatcacttgccgggcaagtcagagcattagcagctatttaaattggtatcagcagaaagcagggaaagcccctaagcttctgatctatgctgcatccagtttgcaaagtggggtcccatcaaggttcagcggcagtggatctgggacagatttcactctcaccatcagctgcctgcagtctgaagatgttgcaacctattactgccaacagtatgaaagttatcgaaggtcgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 141 - DNA, 1395 bp, synthetic construct
  misc_feature 1..1395: note = nucleotide of Chimeric antigen receptor
  source 1..1395: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagctgcctgcagtctgaagattttgcaacttattactgccaacagtataatagttattcccggacgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 142 - DNA, 1395 bp, synthetic construct
  misc_feature 1..1395: note = nucleotide of Chimeric antigen receptor
  source 1..1395: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagattggaaagtggggtcccatcaaggttcagtggcagtggatctgggacagatttcactctcaccatcagctgcctgcagtctgaagattttgcaacttattactgccaacagtataatagttattcccggacgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 143 - DNA, 1398 bp, synthetic construct
  misc_feature 1..1398: note = nucleotide of Chimeric antigen receptor
  source 1..1398: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgctctatgctgcatccagattggaaagtggggtcccatcaaggttcagcggcagtggatctgggacagaattcactctcaccatcagcagcctgcagcctgatgattttgcaacttattactgccaacagtataatagttattctccgtgcactttcggccctgggaccaaagtggatatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 144 - DNA, 1392 bp, synthetic construct
  misc_feature 1..1392: note = nucleotide of Chimeric antigen receptor
  source 1..1392: mol_type = other DNA
gacatccagatgacccagtctccatcctccctgtctgcatctgtaggggacagagtctccatcacttgccgggcaagtcaggacattagcaggtatttaaattggtatcagcagaaaccagggaaagcccctaagctcctgctctatgctgcatccagattggaaagtggggtcccatccaggttcagtggcagtggatctgggacagacttcactctcaccatcaccagcctgcagcctgatgactttgcaacttattactgccaacagtatgataatctgatcaccttcggccaagggacacgactggagattaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 145 - DNA, 1395 bp, synthetic construct
  misc_feature 1..1395: note = nucleotide of Chimeric antigen receptor
  source 1..1395: mol_type = other DNA
gacatccagatgacccagtctccatcctccctgtctgcatctgtaggggacagagtctccatcacttgccgggcaagtcaggacattagcaggtatttaaattggtatcagcagaaaccagggaaagcccctaagctcctgctctatgctgcatccagattggaaagtggggtcccatccaggttcagtggcagtggatctgggacagacttcactctcaccatcaccagcctgcagcctgatgactttgcaacttattactgccaacagtataatagttattcccggacgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 146 - DNA, 1392 bp, synthetic construct
  misc_feature 1..1392: note = nucleotide of Chimeric antigen receptor
  source 1..1392: mol_type = other DNA
gacatccagatgacccagtctccatcctccctgtctgcatctgtaggggacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagacttcactctcaccatcaccagcctgcagcctgatgactttgcaacttattactgccaacagtatgataatctgatcaccttcggccaagggacacgactggagattaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 147 - DNA, 1395 bp, synthetic construct
  misc_feature 1..1395: note = nucleotide of Chimeric antigen receptor
  source 1..1395: mol_type = other DNA
gaaattgtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagactggagcctgaagattttgcaacctattactgccaacagtataatagttattcccggacgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 148 - DNA, 1395 bp, synthetic construct
  misc_feature 1..1395: note = nucleotide of Chimeric antigen receptor
  source 1..1395: mol_type = other DNA
gacatccagatgacccagtctccatcctccctgtctgcatctgtaggagacagagtcaccatcacttgccgggcaagtcagagcattagcagctatttaaattggtatcagcagaaagcagggaaagcccctaagcttctgatctatgctgcatccagtttgcaaagtggggtcccatcaaggttcagcggcagtggatctgggacagatttcactctcaccatcagctgcctgcagtctgaagatgttgcaacctattactgccaacagtatgaaagttattcccggacgttcggccaagggaccaaggtggaaatcaaaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 149 - DNA, 1404 bp, synthetic construct
  misc_feature 1..1404: note = nucleotide of Chimeric antigen receptor
  source 1..1404: mol_type = other DNA
cagtctgccctgactcagcctccctccgcgtccgggtctcctggacagtcagtcaccatctcctgcactggaaccagcagtgacgttggtggttatgactttgtctcctggtaccaacagcacccaggcgaagcccccaaactcctcgtttatgatgtcaataaccggccctcaggggtttctaatcgcttctctggctccaagtctggcaacacggcctccctgaccatctctgggctccaggctgaggacgagggtgactattactgcagctcatatgcaggcagcaacagcgtcttcggaactgggaccaaggtcaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 150 - DNA, 1404 bp, synthetic construct
  misc_feature 1..1404: note = nucleotide of chimeric antigen receptor
  source 1..1404: mol_type = other DNA
gaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcaggctctacaagcggctctggcaagcctggatctggcgagggaagcaccaagggccagtctgccctgactcagcctccctccgcgtccgggtctcctggacagtcagtcaccatctcctgcactggaaccagcagtgacgttggtggttatgactttgtctcctggtaccaacagcacccaggcgaagcccccaaactcctcgtttatgatgtcaataaccggccctcaggggtttctaatcgcttctctggctccaagtctggcaacacggcctccctgaccatctctgggctccaggctgaggacgagggtgactattactgcagctcatatgcaggcagcaacagcgtcttcggaactgggaccaaggtcaccgtcctagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 151 - DNA, 1407 bp, synthetic construct
  misc_feature 1..1407: note = nucleotide of Chimeric antigen receptor
  source 1..1407: mol_type = other DNA
cagtctgccctgactcagcctgcctccgtgtctgggtctcctggacagtcgatcaccatctcctgcactgggaccagcagtgacgttggtggttatgaatttgtctcctggtaccaacaacacccaggcagcgcccccaaactcattatttatgacgtaatagagcgtcccttcggtgtctcctatcggttctctgcctccaagtcaggcaacacggcctccctgacgatctctgggctccagggtgaagacgaggctgattacttctgcagctcatatacaagcagcagcacttatgtcttcggaactgggaccaaggtcaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 152 - DNA, 1407 bp, synthetic construct
  misc_feature 1..1407: note = nucleotide of Chimeric antigen receptor
  source 1..1407: mol_type = other DNA
cagtctgccctgactcagcctgcctccgtgtctgggtctcctggacagtcgatcaccatctcctgcactggaaccggcagtgacgttggtgcttatgactatgtctcctggtaccaacatcacccaggcagagcccccagactcatcattcgtgatgtcagtgtgcggccctcaggggtccctgatcgcttctctggctccaagtctggcaacacggcctccctgaccatctctgggctccaggctgaggacgaggctgattattactgctcctcatattcaggcagcagcacttgggtgttcggcggggggaccaagctgaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 153 - DNA, 1410 bp, synthetic construct
  misc_feature 1..1410: note = nucleotide of Chimeric antigen receptor
  source 1..1410: mol_type = other DNA
cagtctgccctgactcagcctgcctccgtgtctgggtctcctggacagtcgatcaccatctcctgcactggaaccagcagtgatgttgggagttataaccttgtctcctggtaccaacaacacccaggcaaagcccccaaactcatgatttatgatgtcagtaatcggccctcaggggtttcttatcgcttctctggctccaagtctggcaacacggcctccctgaccatctctgggctccaggctgaggacgaggctgattattactgcagctcatatacaagcagcagcactttcgcggtgttcggcggagggacccagctgaccgtcctcggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 154 - DNA, 1404 bp, synthetic construct
  misc_feature 1..1404: note = nucleotide of Chimeric antigen receptor
  source 1..1404: mol_type = other DNA
caatctgccctgcctcagcctgcctccgtgtctgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggttccaagtctggcaacacggcctccctgaccgtctctgggctccaggctgaggatgaggctgattatttctgctgttcgtatgcaggcggctattatgtcttcggaactgggaccaagctgaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 155 - DNA, 1413 bp, synthetic construct
  misc_feature 1..1413: note = nucleotide of Chimeric antigen receptor
  source 1..1413: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccagactgaggacgaggctgattactattgcagctcatatgcaggcagcggcagcaccccctttgtcttcggaactgggaccaagctgaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 156 - DNA, 1404 bp, synthetic construct
  misc_feature 1..1404: note = nucleotide of Chimeric antigen receptor
  source 1..1404: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaagggtccctgatcgcttcgctggctccaagtctggcaacacggcctccctgaccatctctgggctccaggctgaagacgaggctgattattactgctgctcatatgccggcagacgttatgtgttcggaactgggaccaagctgaccgtcctaggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 157 - DNA, 1401 bp, synthetic construct
  misc_feature 1..1401: note = nucleotide of Chimeric antigen receptor
  source 1..1401: mol_type = other DNA
cagctgcagctgcaggagtcgggcccaggactggtgaagccttcacagaccctgtccctcacctgcactgtctctggtggctccatcagtagttactactggagctggatccggcagcccccagggaagggactggagtggattggggaaatcaatcatagtggaagcaccaactacaacccgtccctcaagagtcgagtcaccatatcagtagacacgtctaagaaccagttctccctgaagctgagctctgtgactgccgcggacacggccgtgtattactgtgcgaggtgccctatctactactacggtatggacgtctggggccaagggaccacggtcaccgtctcctcaggctctacaagcggctctggcaagcctggatctggcgagggaagcaccaagggccagagcgagctgacacagcctagatccgtgtctggcagccctggccagagcgtgaccatcagctgtaccggcaccagcagagatgtgggcggctacaactacgtgtcctggtatcagcagcatcccggcaaggcccccaagctgatcatccacgacgtgatcgagcggagcagcggcgtgcccgatagattcagcggcagcaagagcggcaacaccgccagcctgacaatcagcggactgcaggccgaggacgaggccgactactactgttggagcttcgccggcagctactacgtgttcggcaccggcaccgatgtgaccgtgctggcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 158 - protein, 44 aa, Homo sapiens
  VARIANT 1: note = X is any amino acid
  VARIANT 20: note = X is any amino acid
  VARIANT 25: note = X is any amino acid
  VARIANT 44: note = X is any amino acid
XGSTSGSGKPGSGEGSTKGXSGSGXGSTSGSGKPGSGEGSTKGX

SEQ ID NO: 159 - DNA, 124 bp, Homo sapiens
  misc_difference 1: note = n is any nucleotide
  misc_difference 56: note = n is any nucleotide; note = n is any nucleotide
  misc_difference 69: note = n is any nucleotide
  misc_difference 124: note = n is any nucleotide
  source 1..124: mol_type = genomic DNA
nggctctacaagcggctctggcaagcctggatctggcgagggaagcaccaagggcnagcggatctggcnggctctacaagcggctctggcaagcctggatctggcgagggaagcaccaagggcn

SEQ ID NO: 160 - protein, 44 aa, Homo sapiens; identical to SEQ ID NO: 158
  VARIANT 1: note = X is any amino acid
  VARIANT 20: note = X is any amino acid
  VARIANT 25: note = X is any amino acid
  VARIANT 44: note = X is any amino acid

SEQ ID NO: 161 - DNA, 124 bp, Homo sapiens; identical to SEQ ID NO: 159
  misc_difference 1: note = n is any nucleotide
  misc_difference 12..124: note = n is any nucleotide
  misc_difference 56: note = n is any nucleotide
  misc_difference 69: note = n is any nucleotide
  source 1..124: mol_type = genomic DNA

SEQ ID NO: 162 - DNA, 357 bp, synthetic construct
  misc_feature 1..357: note = Nucleotide of 3M4E5 heavy chain
  source 1..357: mol_type = other DNA
gaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctca

SEQ ID NO: 163 - DNA, 327 bp, synthetic construct
  misc_feature 1..327: note = Nucleotide of 3M4E5 light chain
  source 1..327: mol_type = other DNA
cagagcgagctgacacagcctagatccgtgtctggcagccctggccagagcgtgaccatcagctgtaccggcaccagcagagatgtgggcggctacaactacgtgtcctggtatcagcagcatcccggcaaggcccccaagctgatcatccacgacgtgatcgagcggagcagcggcgtgcccgatagattcagcggcagcaagagcggcaacaccgccagcctgacaatcagcggactgcaggccgaggacgaggccgactactactgttggagcttcgccggcagctactacgtgttcggcaccggcaccgatgtgaccgtgctg

SEQ ID NO: 164 - protein, 246 aa, synthetic construct
  REGION 1..246: note = 3M4E5HL scFv
EVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSGSTSGSGKPGSGEGSTKGQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVL

SEQ ID NO: 165 - DNA, 738 bp, synthetic construct
  misc_feature 1..738: note = Nucleotide of 3M4E5HL scFv
  source 1..738: mol_type = other DNA
gaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcaggctctacaagcggctctggcaagcctggatctggcgagggaagcaccaagggccagagcgagctgacacagcctagatccgtgtctggcagccctggccagagcgtgaccatcagctgtaccggcaccagcagagatgtgggcggctacaactacgtgtcctggtatcagcagcatcccggcaaggcccccaagctgatcatccacgacgtgatcgagcggagcagcggcgtgcccgatagattcagcggcagcaagagcggcaacaccgccagcctgacaatcagcggactgcaggccgaggacgaggccgactactactgttggagcttcgccggcagctactacgtgttcggcaccggcaccgatgtgaccgtgctg

SEQ ID NO: 166 - protein, 246 aa, synthetic construct
  REGION 1..246: note = 3M4E5LH scFv
QSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 167 - DNA, 738 bp, synthetic construct
  misc_feature 1..738: note = Nucleotide of 3M4E5LH scFv
  source 1..738: mol_type = other DNA
cagagcgagctgacacagcctagatccgtgtctggcagccctggccagagcgtgaccatcagctgtaccggcaccagcagagatgtgggcggctacaactacgtgtcctggtatcagcagcatcccggcaaggcccccaagctgatcatccacgacgtgatcgagcggagcagcggcgtgcccgatagattcagcggcagcaagagcggcaacaccgccagcctgacaatcagcggactgcaggccgaggacgaggccgactactactgttggagcttcgccggcagctactacgtgttcggcaccggcaccgatgtgaccgtgctgggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctca

SEQ ID NO: 168 - protein, 110 aa, Homo sapiens
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 169 - DNA, 330 bp, Homo sapiens
  source 1..330: mol_type = genomic DNA
gcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatct

SEQ ID NO: 170 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 336 bp, Homo sapiens
  source 1..336: mol_type = genomic DNA
cgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 172 - protein, 4 aa, synthetic construct
  REGION 1..4: note = furin cleavage site
RAKR

SEQ ID NO: 173 - protein, 4 aa, synthetic construct
  REGION 1..4: note = Spacer sequence
SGSG

SEQ ID NO: 174 - protein, 19 aa, synthetic construct
  REGION 1..19: note = Codon-optimized P2A sequence
ATNFSLLKQAGDVEENPGP

SEQ ID NO: 175 - DNA, 45 bp, synthetic construct
  misc_feature 1..45: note = 5'-RACE primer
  source 1..45: mol_type = other DNA
ctaatacgactcactatagggcaagcagtggtatcaacgcagagt

SEQ ID NO: 176 - DNA, 21 bp, synthetic construct
  misc_feature 1..21: note = 3'IGHC primer
  source 1..21: mol_type = other DNA
tgagttccacgacaccgtcac

SEQ ID NO: 177 - DNA, 22 bp, synthetic construct
  misc_feature 1..22: note = 3'IGHM primer
  source 1..22: mol_type = other DNA
tccaggacaaagtgatggagtc

SEQ ID NO: 178 - DNA, 43 bp, synthetic construct
  misc_feature 1..43: note = Modified 5'-RACE primer
  source 1..43: mol_type = other DNA
gtgtggtggtacgggaattcaagcagtggtatcaacgcagagt

SEQ ID NO: 179 - DNA, 44 bp, synthetic construct
  misc_feature 1..44: note = 3'IGHJ1 primer
  source 1..44: mol_type = other DNA
cttgccagagccgcttgtagagcctgaggagacggtgaccaggg

SEQ ID NO: 180 - DNA, 46 bp, synthetic construct
  misc_feature 1..46: note = 3'IGHJ2 primer
  source 1..46: mol_type = other DNA
cttgccagagccgcttgtagagcctgaagagacggtgaccattgtc

SEQ ID NO: 181 - DNA, 46 bp, synthetic construct
  misc_feature 1..46: note = 3'IGHJ3 primer
  source 1..46: mol_type = other DNA
cttgccagagccgcttgtagagcctgaggagacggtgaccgtggtc

SEQ ID NO: 182 - DNA, 22 bp, synthetic construct
  misc_feature 1..22: note = 3'IGKC primer
  source 1..22: mol_type = other DNA
agggtcagaggccaaaggatgg

SEQ ID NO: 183 - DNA, 20 bp, synthetic construct
  misc_feature 1..20: note = 3'IGLC primer
  source 1..20: mol_type = other DNA
cttggagctcctcagaggag

SEQ ID NO: 184 - DNA, 45 bp, synthetic construct
  misc_feature 1..45: note = 3'IGKJ1 primer
  source 1..45: mol_type = other DNA
cttgccagagccgcttgtagagcctttgatttccaccttggtccc

SEQ ID NO: 185 - DNA, 45 bp, synthetic construct
  misc_feature 1..45: note = 3'IGKJ2 primer
  source 1..45: mol_type = other DNA
cttgccagagccgcttgtagagcctttgatctccagcttggtccc

SEQ ID NO: 186 - DNA, 45 bp, synthetic construct
  misc_feature 1..45: note = 3'IGKJ3 primer
  source 1..45: mol_type = other DNA
cttgccagagccgcttgtagagcctttgatatccactttggtccc

SEQ ID NO: 187 - DNA, 45 bp, synthetic construct
  misc_feature 1..45: note = 3'IGKJ4 primer
  source 1..45: mol_type = other DNA
cttgccagagccgcttgtagagcctttaatctccagtcgtgtccc

SEQ ID NO: 188 - DNA, 45 bp, synthetic construct
  misc_feature 1..45: note = 3'IGLJ1 primer
  source 1..45: mol_type = other DNA
cttgccagagccgcttgtagagcctaggacggtgaccttggtccc

SEQ ID NO: 189 - DNA, 45 bp, synthetic construct
  misc_feature 1..45: note = 3'IGLJ2 primer
  source 1..45: mol_type = other DNA
cttgccagagccgcttgtagagcctaggacggtcagcttggtccc

SEQ ID NO: 190 - DNA, 45 bp, synthetic construct
  misc_feature 1..45: note = 3'IGLJ4 primer
  source 1..45: mol_type = other DNA
cttgccagagccgcttgtagagcctaaaatgatcagctgggttcc

SEQ ID NO: 191 - DNA, 45 bp, synthetic construct
  misc_feature 1..45: note = 3'IGLJ5 primer
  source 1..45: mol_type = other DNA
cttgccagagccgcttgtagagcctaggacggtcagctcggtccc

SEQ ID NO: 192 - DNA, 45 bp, synthetic construct
  misc_feature 1..45: note = 3'IGLJ7 primer
  source 1..45: mol_type = other DNA
cttgccagagccgcttgtagagccgaggacggtcagctgggtgcc

SEQ ID NO: 193 - DNA, 1095 bp, Homo sapiens
  source 1..1095: mol_type = genomic DNA
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtg

SEQ ID NO: 194 - DNA, 864 bp, Homo sapiens
  source 1..864: mol_type = genomic DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 195 - DNA, 615 bp, Homo sapiens
  source 1..615: mol_type = genomic DNA
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactcctcacaagacagaactggta

SEQ ID NO: 196 - DNA, 831 bp, Homo sapiens
  source 1..831: mol_type = genomic DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 197 - DNA, 762 bp, Homo sapiens
  source 1..762: mol_type = genomic DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 198 - protein, 9 aa, Homo sapiens
SLYNTVATL

SEQ ID NO: 199 - DNA, 21 bp, synthetic construct
  misc_feature 1..21: note = F primer 1
  source 1..21: mol_type = other DNA
atcccagtgtggtggtacggg

SEQ ID NO: 200 - DNA, 20 bp, synthetic construct
  misc_feature 1..20: note = R primer 1
  source 1..20: mol_type = other DNA
gggtacatcacttcgattgc

SEQ ID NO: 201 - protein, 245 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 202 - DNA, 735 bp, Homo sapiens
  source 1..735: mol_type = genomic DNA
gaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgctatggactattggggccagggcaccagcgttacagtgtcctcgggctctacaagcggctctggcaagcctggatctggcgagggaagcaccaagggcgatatccagatgacccagacaacaagcagcctgagcgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcaca

SEQ ID NO: 203 - protein, 245 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 204 - DNA, 735 bp, Homo sapiens
  source 1..735: mol_type = genomic DNA
gatatccagatgacccagacaacaagcagcctgagcgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggctctacaagcggctctggcaagcctggatctggcgagggaagcaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgctatggactattggggccagggcaccagcgttacagtgtcctcg

SEQ ID NO: 205 - DNA, 1668 bp, Homo sapiens
  source 1..1668: mol_type = genomic DNA
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccagg

SEQ ID NO: 206 - protein, 9 aa, Homo sapiens
SLLMWITQC

SEQ ID NO: 207 - protein, 9 aa, synthetic construct
  REGION 1..9: note = Peptide
GLRMWIKQV

SEQ ID NO: 208 - DNA, 711 bp, synthetic construct
  misc_feature 1..711: note = synthesized oligonucleotides
  source 1..711: mol_type = other DNA
atggcggctgcatctggaagcgtgctgcagagatgtatcgtgtccccagccggcagacatagcgccagcctgatttttctgcacggcagcggcgattctggccagggactgagaatgtggatcaaacaggtgctgaaccaggacctgaccttccagcacatcaagatcatctaccccaccgctccacctcggagctacacacctatgaagggcggcatcagcaacgtttggttcgaccggttcaagatcaccaacgactgccccgagcacctggaatccatcgacgtgatgtgtcaggtgctcaccgacctgatcgacgaggaagtgaagtccggcatcaagaagaacagaatcctgatcggcggcttcagcatgggcggctgtatggccattcacctggcctacagaaaccaccaggatgtggctggcgtgttcgccctgagcagctttctgaacaaagccagcgccgtgtatcaggccctgcagaaatctaacggcgtgctgcctgagctgttccagtgtcatggcacagccgatgagctggtgctgcactcttgggccgaagagacaaatagcatgctgaaaagcctgggcgtgaccaccaagttccacagcttccccaacgtgtaccacgagctgagcaagaccgagctggacatcctgaaactgtggattctgaccaagctgcccggcgagatggaaaagcagaag

SEQ ID NO: 209 - DNA, 540 bp, synthetic construct
  misc_feature 1..540: note = synthesized oligonucleotides
  source 1..540: mol_type = other DNA
atgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgc

SEQ ID NO: 210 - protein, 470 aa, synthetic construct
  REGION 1..470: note = Chimeric antigen receptor
QSVLTQPPSVSGAPGQRVTISCTGSSSNLGAGYDVHWYQQLPGTAPKLLIYGSTTRPSGIPDRFSGSKSGTSATLGITGLQTGDEADYYCGTWDSSLSAGVFGGGTQLTVLGSTSGSGKPGSGEGSTKGEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - DNA, 1410 bp, synthetic construct
  misc_feature 1..1410: note = synthesized oligonucleotides
  source 1..1410: mol_type = other DNA
cagtctgtgctgacgcagccgccctcagtgtctggggccccagggcagagggtcaccatctcctgcactgggagcagctccaacctcggggcaggttatgatgtacactggtaccagcagcttccaggaacagcccccaaactcctcatctatggtagcaccactcggccctcagggattcctgaccgattctctggctccaagtctggcacgtcagccaccctgggcatcaccggactccagactggggacgaggccgattattactgcggaacatgggatagcagcctgagtgctggggtgttcggcggaggcacccagctgaccgtcctcggctctacaagcggctctggcaagcctggatctggcgagggaagcaccaagggcgaagtgcagctgctggaatctggcggcggactggtgcagcctggcggatctctgagactgagctgtgccgccagcggcttcaccttcagcacctaccagatgagctgggtgcgccaggcccctggcaaaggactggaatgggtgtccggcatcgtgtccagcggcggctctacagcctacgccgatagcgtgaagggccggttcaccatcagccgggacaacagcaagaacaccctgtacctgcagatgaacagcctgagagccgaggacaccgccgtgtactattgtgccggggagctgctgccctactacggcatggatgtgtggggccagggcaccaccgtgacagtgtcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 212 - protein, 467 aa, synthetic construct
  REGION 1..467: note = Chimeric antigen receptor
EIVLTQSPATLSVSPGERATLSCRASQSVSSNLAWYQQKPGQAPRLLIYGASTRATGIPARFSGSGSGTEFTLTISRLEPEDFAVYYCQQYNNWPPKFTFGPGTKVDIRGSTSGSGKPGSGEGSTKGQVQLQESGPGLVKPSDTLSLTCLVSGGSISSNYWSWIRQAPGKGLEWIGHVSYSGSTNYNPSLKSRVTISVDTSKNQFSLKLSSVTAADTAVYYCARESYYYYGMDVWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - DNA, 1401 bp, synthetic construct
  misc_feature 1..1401: note = synthesized oligonucleotides
  source 1..1401: mol_type = other DNA
gaaattgtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagactggagcctgaagattttgcagtgtattactgtcagcagtataataactggcctccgaaattcactttcggccctgggaccaaagtggatatcagaggctctacaagcggctctggcaagcctggatctggcgagggaagcaccaagggccaggtgcagctgcaggagtcgggcccaggactggtgaagccttcggataccctgtccctcacctgtcttgtctctggtggctccatcagtagtaattactggagctggatccggcaggccccagggaagggactggagtggattggacatgtctcctacagtgggagcaccaactacaacccctccctcaagagtcgagttaccatatcagtagacacgtctaagaaccagttctccctgaagctgagctctgtgactgccgcggacacggccgtgtattactgtgcgagagagtcctactactactacggtatggacgtctggggccaagggaccacggtcaccgtctcctcagcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 214 - protein, 249 aa, synthetic construct
  REGION 1..249: note = single chain Fv
QSALTQPRSVSGFPGQSVTISCTGTTSDDVSWYQQHPGKAPQLMLYDVSKRPSGVPHRFSGSRSGRAASLIISGLQTEDEADYFCCSYAGRYNSVLFGGGTKLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 215 - DNA, 747 bp, synthetic construct
  misc_feature 1..747: note = synthesized oligonucleotides
  source 1..747: mol_type = other DNA
caatctgctctgacacagcctagaagcgtgtccggctttcctggccagagcgtgaccatcagctgtaccggcaccacctccgatgacgtgtcctggtatcagcagcatcctggcaaagcccctcagctgatgctgtacgacgtgtccaaaagacctagcggcgtgccccacagattcagcggatctagaagtggcagagccgccagcctgatcatctctggactgcagacagaggacgaggccgactacttctgctgtagctacgccggcagatacaacagcgtgctgtttggcggcggaacaaagctgacagtgctgggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttct

SEQ ID NO: 216 - protein, 8 aa, Homo sapiens
GFTFDDYA

SEQ ID NO: 217 - protein, 8 aa, Homo sapiens
ISWNSGRI

SEQ ID NO: 218 - protein, 18 aa, Homo sapiens
ARDQGYHYYDSAEHAFDI

SEQ ID NO: 219 - protein, 125 aa, Homo sapiens
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 220 - protein, 6 aa, Homo sapiens
KLGDKY

SEQ ID NO: 221 - skipped

SEQ ID NO: 222 - protein, 10 aa, Homo sapiens
QAWDSSTHVV

SEQ ID NO: 223 - protein, 107 aa, Homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTHVVFGGGTKLTVL

SEQ ID NO: 224 - protein, 9 aa, Homo sapiens
SSDVGGYNY

SEQ ID NO: 225 - skipped

SEQ ID NO: 226 - protein, 11 aa, Homo sapiens
GTWDTSLTAVV

SEQ ID NO: 227 - protein, 111 aa, Homo sapiens
QSALTQPRSVSGSPGQSVTISCTGTSSDVGGYNYVSWYQQHPGKAPKLMIYDVSNRPSGVSNRFSGSKSGNTASLTISGLQTGDEADYYCGTWDTSLTAVVFGGGTELTVL

SEQ ID NO: 228 - protein, 8 aa, Homo sapiens
WSNIGDDH

SEQ ID NO: 229 - skipped

SEQ ID NO: 230 - protein, 11 aa, Homo sapiens
GTWESSLSGVV

SEQ ID NO: 231 - protein, 110 aa, Homo sapiens
QSVLTQPPSVSAAPGQKVTISCSGSWSNIGDDHVSWYQQFPGAAPKLLIYDTSKRPSRVADRFSGSKSGASATLAITGLQAGDEADYYCGTWESSLSGVVFGGGTELTVL

SEQ ID NO: 232 - protein, 9 aa, Homo sapiens
SSDVGGYDY

SEQ ID NO: 233 - skipped

SEQ ID NO: 234 - protein, 10 aa, Homo sapiens
SSYTTSTTWV

SEQ ID NO: 235 - protein, 111 aa, Homo sapiens; identical to SEQ ID NO: 227

SEQ ID NO: 236 - protein, 9 aa, Homo sapiens
TSDVGTTNY

SEQ ID NO: 237 - skipped

SEQ ID NO: 238 - protein, 10 aa, Homo sapiens
SYAGSYTFVV

SEQ ID NO: 239 - protein, 111 aa, Homo sapiens
QSALTQPASVSGSPGQSITISCTGTTSDVGTTNYVSWYQQHPGKAPKLLIYDVTNRPSGVPDRFSGSKSANTASLTISGLQAEDEADYYCCSYAGSYTFVVFGGGTELTVL

SEQ ID NO: 240 - protein, 9 aa, Homo sapiens
SSDVGVYNY

SEQ ID NO: 241 - skipped

SEQ ID NO: 242 - protein, 11 aa, Homo sapiens
AAWDDSLNGVV

SEQ ID NO: 243 - protein, 111 aa, Homo sapiens
QSALTQPPSASGSPGQSVTISCTGTSSDVGVYNYVSWYQQHPGKAPKLMIYDVSKRPSGVPDRFSGSKSANTASLTISGLQAEDEADYYCAAWDDSLNGVVFGGGTQLTVL

SEQ ID NO: 244 - protein, 8 aa, Homo sapiens
SSNIGNNY

SEQ ID NO: 245 - skipped

SEQ ID NO: 246 - protein, 10 aa, Homo sapiens
AAWDDSLSAI

SEQ ID NO: 247 - protein, 109 aa, Homo sapiens
QSVLTQPPSASGTPGQRVTISCSGSSSNIGNNYVCWYQHLPGTAPKLLIYDNVKRPSGIPDRFSGSKSGTSASLAISGLRSEDEADYYCAAWDDSLSAIFGGGTELTVL

SEQ ID NO: 248 - protein, 9 aa, Homo sapiens
SSDVGGYNY

SEQ ID NO: 249 - skipped

SEQ ID NO: 250 - protein, 10 aa, Homo sapiens
HSYDSSLSHV

SEQ ID NO: 251 - protein, 110 aa, Homo sapiens
QSALTQPASVSGSPGQSITISCTGTSSDVGGYNYVSWYQQHPGKAPKLMIYDVSNRPSGVPDRFSGSKSGTSASLAISGLQSEDEADYYCHSYDSSLSHVFGTGTKVTVL

SEQ ID NO: 252 - protein, 6 aa, Homo sapiens
QSVSSN

SEQ ID NO: 253 - skipped

SEQ ID NO: 254 - protein, 11 aa, Homo sapiens
QQYNNWPPLYT

SEQ ID NO: 255 - protein, 109 aa, Homo sapiens
EIVLTQSPGTLSLSPGERATLSCRASQSVSSNLAWYQQKPGQAPRLLIYGASTRATGIPARFSGSGSGTEFTLTISSLQSEDFAVYYCQQYNNWPPLYTFGQGTKLEIK

SEQ ID NO: 256 - protein, 6 aa, Homo sapiens
QSVSSY

SEQ ID NO: 257 - skipped

SEQ ID NO: 258 - protein, 10 aa, Homo sapiens
QQSYSTLLYT

SEQ ID NO: 259 - protein, 108 aa, Homo sapiens
EIVLTQSPGTLSLSPGERATLSCRASQSVSSYLAWYQQKPGQAPRLLIYDASNRATGIPARFSGSGSGTDFTLTISSLEPEDFATYYCQQSYSTLLYTFGQGTKLEIK

SEQ ID NO: 260 - protein, 6 aa, Homo sapiens
QSVSSY

SEQ ID NO: 261 - skipped

SEQ ID NO: 262 - protein, 9 aa, Homo sapiens
QQYDSLPLT

SEQ ID NO: 263 - protein, 107 aa, Homo sapiens
EIVLTRSPATLSLSPGERATLSCRASQSVSSYLAWYQQKPGQAPRLLIYDASNRATGISARFSGSGSGTEFTLTISSLQSEDIATYYCQQYDSLPLTFGGGTKLEIK

SEQ ID NO: 264 - protein, 7 aa, Homo sapiens
QTISASS

SEQ ID NO: 265 - skipped

SEQ ID NO: 266 - protein, 9 aa, Homo sapiens
QQFNEWPLT

SEQ ID NO: 267 - protein, 108 aa, Homo sapiens
EIVLTQSPATLSLSPGERATVSCRPSQTISASSVAWYQQKAGQAPRLLIYGASSRATGIPDRFSGSGSGTDFTLTISRLEPEDFAVYYCQQFNEWPLTFGGGTKVEIK

SEQ ID NO: 268 - protein, 6 aa, Homo sapiens
QSVSSN

SEQ ID NO: 269 - skipped

SEQ ID NO: 270 - protein, 11 aa, Homo sapiens
QQYGSSPDIFT

SEQ ID NO: 271 - protein, 109 aa, Homo sapiens
EIVLTQSPATLSLSPGERATLSCRASQSVSSNLAWYQQKLGQAPRLLIYGASSRATGIPDRFSGSGSGTDFTLTISRLEPEDFAVYYCQQYGSSPDIFTFGPGTKVDIK

SEQ ID NO: 272 - protein, 250 aa, synthetic construct
  REGION 1..250: note = Anitgen binding domain of L4-18H
SYELTQPPSVSVSPGQTASITCSGDKLGDKYACWYQQKPGQSPVLVIYQDSKRPSGIPERFSGSNSGNTATLTISGTQAMDEADYYCQAWDSSTHVVFGGGTKLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 273 - protein, 254 aa, synthetic construct
  REGION 1..254: note = Anitgen binding domain of L7-18H
QSALTQPRSVSGSPGQSVTISCTGTSSDVGGYNYVSWYQQHPGKAPKLMIYDVSNRPSGVSNRFSGSKSGNTASLTISGLQTGDEADYYCGTWDTSLTAVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 274 - protein, 253 aa, synthetic construct
  REGION 1..253: note = Anitgen binding domain of L9-18H
QSVLTQPPSVSAAPGQKVTISCSGSWSNIGDDHVSWYQQFPGAAPKLLIYDTSKRPSRVADRFSGSKSGASATLAITGLQAGDEADYYCGTWESSLSGVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 275 - protein, 253 aa, synthetic construct
  REGION 1..253: note = Anitgen binding domain of L13-18H
QSALTQPASVSGSPGQSITISCTGLSSDVGGYDYVSWYQQHPGIAPKLMIYDVTNRPSGVSSRFSGSKSGNTASLTISGLQAEDEADYYCSSYTTSTTWVFGAGTKLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 276 - protein, 254 aa, synthetic construct
  REGION 1..254: note = Anitgen binding domain of L14-18H
QSALTQPASVSGSPGQSITISCTGTTSDVGTTNYVSWYQQHPGKAPKLLIYDVTNRPSGVPDRFSGSKSANTASLTISGLQAEDEADYYCCSYAGSYTFVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 277 - protein, 254 aa, synthetic construct
  REGION 1..254: note = Anitgen binding domain of L16-18H
QSALTQPPSASGSPGQSVTISCTGTSSDVGVYNYVSWYQQHPGKAPKLMIYDVSKRPSGVPDRFSGSKSANTASLTISGLQAEDEADYYCAAWDDSLNGVVFGGGTQLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 278 - protein, 252 aa, synthetic construct
  REGION 1..252: note = Anitgen binding domain of L17-18H
QSVLTQPPSASGTPGQRVTISCSGSSSNIGNNYVCWYQHLPGTAPKLLIYDNVKRPSGIPDRFSGSKSGTSASLAISGLRSEDEADYYCAAWDDSLSAIFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 279 - protein, 253 aa, synthetic construct
  REGION 1..253: note = Anitgen binding domain of L22-18H
QSALTQPASVSGSPGQSITISCTGTSSDVGGYNYVSWYQQHPGKAPKLMIYDVSNRPSGVPDRFSGSKSGTSASLAISGLQSEDEADYYCHSYDSSLSHVFGTGTKVTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 280 - protein, 252 aa, synthetic construct
  REGION 1..252: note = Anitgen binding domain of K4-18H
EIVLTQSPGTLSLSPGERATLSCRASQSVSSNLAWYQQKPGQAPRLLIYGASTRATGIPARFSGSGSGTEFTLTISSLQSEDFAVYYCQQYNNWPPLYTFGQGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 281 - protein, 251 aa, synthetic construct
  REGION 1..251: note = Anitgen binding domain of K5-18H
EIVLTQSPGTLSLSPGERATLSCRASQSVSSYLAWYQQKPGQAPRLLIYDASNRATGIPARFSGSGSGTDFTLTISSLEPEDFATYYCQQSYSTLLYTFGQGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 282 - protein, 250 aa, synthetic construct
  REGION 1..250: note = Anitgen binding domain of K6-18H
EIVLTRSPATLSLSPGERATLSCRASQSVSSYLAWYQQKPGQAPRLLIYDASNRATGISARFSGSGSGTEFTLTISSLQSEDIATYYCQQYDSLPLTFGGGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 283 - protein, 251 aa, synthetic construct
  REGION 1..251: note = Anitgen binding domain of K9-18H
EIVLTQSPATLSLSPGERATVSCRPSQTISASSVAWYQQKAGQAPRLLIYGASSRATGIPDRFSGSGSGTDFTLTISRLEPEDFAVYYCQQFNEWPLTFGGGTKVEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 284 - protein, 252 aa, synthetic construct
  REGION 1..252: note = Anitgen binding domain of K10-18H
EIVLTQSPATLSLSPGERATLSCRASQSVSSNLAWYQQKLGQAPRLLIYGASSRATGIPDRFSGSGSGTDFTLTISRLEPEDFAVYYCQQYGSSPDIFTFGPGTKVDIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 285 - protein, 472 aa, synthetic construct
  REGION 1..472: note = L4-18H CAR
SYELTQPPSVSVSPGQTASITCSGDKLGDKYACWYQQKPGQSPVLVIYQDSKRPSGIPERFSGSNSGNTATLTISGTQAMDEADYYCQAWDSSTHVVFGGGTKLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 286 - protein, 476 aa, synthetic construct
  REGION 1..476: note = L7-18H CAR
QSALTQPRSVSGSPGQSVTISCTGTSSDVGGYNYVSWYQQHPGKAPKLMIYDVSNRPSGVSNRFSGSKSGNTASLTISGLQTGDEADYYCGTWDTSLTAVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 287 - protein, 475 aa, synthetic construct
  REGION 1..475: note = L9-18H CAR
QSVLTQPPSVSAAPGQKVTISCSGSWSNIGDDHVSWYQQFPGAAPKLLIYDTSKRPSRVADRFSGSKSGASATLAITGLQAGDEADYYCGTWESSLSGVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 288 - protein, 475 aa, synthetic construct
  REGION 1..475: note = L13-18H CAR
QSALTQPASVSGSPGQSITISCTGLSSDVGGYDYVSWYQQHPGIAPKLMIYDVTNRPSGVSSRFSGSKSGNTASLTISGLQAEDEADYYCSSYTTSTTWVFGAGTKLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 289 - protein, 476 aa, synthetic construct
  REGION 1..476: note = L14-18H CAR
QSALTQPASVSGSPGQSITISCTGTTSDVGTTNYVSWYQQHPGKAPKLLIYDVTNRPSGVPDRFSGSKSANTASLTISGLQAEDEADYYCCSYAGSYTFVVFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 290 - protein, 476 aa, synthetic construct
  REGION 1..476: note = L16-18H CAR
QSALTQPPSASGSPGQSVTISCTGTSSDVGVYNYVSWYQQHPGKAPKLMIYDVSKRPSGVPDRFSGSKSANTASLTISGLQAEDEADYYCAAWDDSLNGVVFGGGTQLTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 291 - protein, 474 aa, synthetic construct
  REGION 1..474: note = L17-18H CAR
QSVLTQPPSASGTPGQRVTISCSGSSSNIGNNYVCWYQHLPGTAPKLLIYDNVKRPSGIPDRFSGSKSGTSASLAISGLRSEDEADYYCAAWDDSLSAIFGGGTELTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 292 - protein, 475 aa, synthetic construct
  REGION 1..475: note = L22-18H CAR
QSALTQPASVSGSPGQSITISCTGTSSDVGGYNYVSWYQQHPGKAPKLMIYDVSNRPSGVPDRFSGSKSGTSASLAISGLQSEDEADYYCHSYDSSLSHVFGTGTKVTVLGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 474 aa, synthetic construct
  REGION 1..474: note = K4-18H CAR
EIVLTQSPGTLSLSPGERATLSCRASQSVSSNLAWYQQKPGQAPRLLIYGASTRATGIPARFSGSGSGTEFTLTISSLQSEDFAVYYCQQYNNWPPLYTFGQGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 294 - protein, 473 aa, synthetic construct
  REGION 1..473: note = K5-18H CAR
EIVLTQSPGTLSLSPGERATLSCRASQSVSSYLAWYQQKPGQAPRLLIYDASNRATGIPARFSGSGSGTDFTLTISSLEPEDFATYYCQQSYSTLLYTFGQGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 472 aa, synthetic construct
  REGION 1..472: note = K6-18H CAR
EIVLTRSPATLSLSPGERATLSCRASQSVSSYLAWYQQKPGQAPRLLIYDASNRATGISARFSGSGSGTEFTLTISSLQSEDIATYYCQQYDSLPLTFGGGTKLEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - protein, 473 aa, synthetic construct
  REGION 1..473: note = K9-18H CAR
EIVLTQSPATLSLSPGERATVSCRPSQTISASSVAWYQQKAGQAPRLLIYGASSRATGIPDRFSGSGSGTDFTLTISRLEPEDFAVYYCQQFNEWPLTFGGGTKVEIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 297 - protein, 474 aa, synthetic construct
  REGION 1..474: note = K10-18H CAR
EIVLTQSPATLSLSPGERATLSCRASQSVSSNLAWYQQKLGQAPRLLIYGASSRATGIPDRFSGSGSGTDFTLTISRLEPEDFAVYYCQQYGSSPDIFTFGPGTKVDIKGSTSGSGKPGSGEGSTKGEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 298 - DNA, 1416 bp, synthetic construct
  misc_feature 1..1416: note = Polynucleotide coding L4-18H CAR
  source 1..1416: mol_type = other DNA
tcctatgagctgactcagccaccctcagtgtccgtgtctccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcacacatgtggtattcggcggagggaccaagctgaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 299 - DNA, 1428 bp, synthetic construct
  misc_feature 1..1428: note = Polynucleotide coding L7-18H CAR
  source 1..1428: mol_type = other DNA
cagtctgccctgactcagcctcgctcagtgtccgggtctcctggacagtcagtcaccatctcctgcactggaaccagcagtgatgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccagactggggacgaggccgattattactgcggaacatgggataccagcctgactgctgtggtattcggcggagggaccgagctgaccgtcctcggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 300 - DNA, 1425 bp, synthetic construct
  misc_feature 1..1425: note = Polynucleotide coding L9-18H CAR
  source 1..1425: mol_type = other DNA
cagtctgtgttgacgcagccgccctcagtgtctgcggccccaggacagaaggtcaccatctcctgctctggaagctggtccaacattggagatgatcatgtctcctggtaccagcagttcccaggagcagcccccaaactcctcatttatgacacttctaagcgaccctcacgcgttgctgaccgattctctggctccaagtctggcgcgtcagccaccctggccatcactggactccaggctggggacgaggccgactattattgcggaacatgggaaagcagcctgagtggtgtggttttcggcggagggaccgagctgaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 301 - DNA, 1425 bp, synthetic construct
  misc_feature 1..1425: note = Polynucleotide coding L13-18H CAR
  source 1..1425: mol_type = other DNA
cagtctgccctgactcagcctgcctccgtgtctgggtctcctggacagtcgatcaccatctcctgcactggactcagcagtgacgttggtggttatgactatgtctcctggtaccaacaacacccaggcatagcccccaaactcatgatttatgatgtcactaatcggccctcaggggtttctagtcgcttctctggctccaagtctggcaacacggcctccctgaccatctctgggctccaggctgaggacgaggctgattattactgcagctcatatacaaccagcacgacttgggtcttcggagctgggaccaagctgaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 302 - DNA, 1428 bp, synthetic construct
  misc_feature 1..1428: note = Polynucleotide coding L14-18H CAR
  source 1..1428: mol_type = other DNA
cagtctgccctgactcagcctgcctccgtgtctgggtctcctggacagtcgatcaccatctcctgcactggaaccaccagtgacgttggtactactaattatgtctcctggtaccagcaacacccaggcaaagcccccaaactcctaatttatgatgtcactaatcggccctcaggggtccctgatcgcttctctggctccaagtctgccaacacggcctccctgaccatctctgggctccaggctgaggacgaggctgattattactgctgctcatatgcaggcagctacaccttcgtggtattcggcggagggaccgagctgaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 303 - DNA, 1428 bp, synthetic construct
  misc_feature 1..1428: note = Polynucleotide coding L16-18H CAR
  source 1..1428: mol_type = other DNA
cagtctgccctgactcagcctccctccgcgtccgggtctcctggacagtcagtcaccatctcctgcactggaaccagcagtgacgttggtgtttataactatgtctcctggtaccaacaacacccaggcaaagcccccaaactcatgatttatgatgtcagtaagcggccctcaggggtccctgatcgcttctctggctccaagtctgccaacacggcctccctgaccatctctgggctccaggctgaggatgaggctgattattactgtgcagcatgggatgacagcctgaatggtgtggtattcggcggaggcacccagctgaccgtcctcggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 304 - DNA, 1422 bp, synthetic construct
  misc_feature 1..1422: note = Polynucleotide coding L17-18H CAR
  source 1..1422: mol_type = other DNA
cagtctgtactgactcaaccaccctcagcgtctgggacccccgggcagagggtcaccatctcttgctctggcagcagctccaacatcggaaataattatgtatgctggtaccaacacctcccaggaacggcccccaaacttctcatttatgacaatgttaagcgaccctcagggattcctgaccgattctctggctccaagtctggcacgtcagcctccctggccatcagtgggctccggtccgaggatgaggctgattattactgtgcagcatgggatgacagcctgagtgccatattcggcggagggaccgagctgaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 305 - DNA, 1425 bp, synthetic construct
  misc_feature 1..1425: note = Polynucleotide coding L22-18H CAR
  source 1..1425: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtccctgatcgcttctctggctccaagtctggcacctcagcctccctggccatcagtgggctccagtctgaggatgaggctgattattactgccactcctatgacagcagcctgagtcatgtcttcggaactgggaccaaggtcaccgtcctaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 306 - DNA, 1422 bp, synthetic construct
  misc_feature 1..1422: note = Polynucleotide coding K4-18H CAR
  source 1..1422: mol_type = other DNA
gaaattgtgttgacgcagtctccaggcaccctgtctttgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcccttgtacacttttggccaggggaccaagctggagatcaaaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 307 - DNA, 1419 bp, synthetic construct
  misc_feature 1..1419: note = Polynucleotide coding K5-18H CAR
  source 1..1419: mol_type = other DNA
gaaattgtgttgacgcagtctccaggcaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcaacttactactgtcaacagagttacagtacccttttgtacacttttggccaggggaccaagctggagatcaaaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 308 - DNA, 1416 bp, synthetic construct
  misc_feature 1..1416: note = Polynucleotide coding K6-18H CAR
  source 1..1416: mol_type = other DNA
gaaattgtgttgacacggtctccagccaccctgtctttgtctccaggggaaagagccaccctctcctgcagggccagtcagagtgttagcagctacttagcctggtaccaacagaaacctggccaggctcccaggctcctcatctatgatgcctccaacagggccactggtatttcagccaggttcagtggcagtgggtctgggacagagttcactctcaccatcagcagcctgcagtctgaagatattgcaacatattactgtcaacagtatgatagtctcccactcactttcggcggagggaccaagctggagatcaaaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 309 - DNA, 1419 bp, synthetic construct
  misc_feature 1..1419: note = Polynucleotide coding K9-18H CAR
  source 1..1419: mol_type = other DNA
gaaatagtgttgacacagtctccagccaccctgtctttgtctccaggggaaagagccaccgtctcctgtaggcccagtcagaccattagtgccagttccgtagcctggtatcagcagaaagctggccaggctccacggctcctcatctatggtgcatccagcagggccactggcatcccagacaggttcagtggcagtgggtctgggacagacttcactctcaccatcagcagactggagcctgaagattttgcagtttattactgtcagcaatttaatgaatggcctctcactttcggcggagggaccaaggtggaaatcaaaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 310 - DNA, 1422 bp, synthetic construct
  misc_feature 1..1422: note = Polynucleotide coding K10-18H CAR
  source 1..1422: mol_type = other DNA
gaaattgtgttgacacagtctccagccaccctgtctttgtctccaggggaaagagccaccctctcctgcagggccagtcagagtgttagcagcaacttagcctggtaccagcaaaaacttggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccgatatattcactttcggccctgggaccaaagtggatatcaaaggctctacaagcggctctggcaagcctggatctggcgagggaagcaccaagggcgaagtgcagcttgtggaatctggcggaggactggtgcagcctggaagaagcctgagactgtcttgtgccgccagcggcttcaccttcgacgattatgccatgcactgggtccgacaggcccctggaaaaggccttgaatgggtgtccggcatctcttggaacagcggcagaatcggctacgccgactctgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgttagttctgcggccgcaatcgaagtgatgtacccccctccctacctggacaacgagaagtccaacggcaccattatccacgtgaagggaaagcacctgtgccccagccctctgttccctggccctagcaagcctttctgggtgctggtggtcgtgggcggagtgctggcctgttatagcctgctcgtgaccgtggccttcatcatcttttgggtgcgcagcaagcggagccggctgctgcacagcgactacatgaacatgacccccagacggcccggacccaccagaaagcactaccagccttacgcccctcccagagacttcgccgcctacagatctcgagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccaga

SEQ ID NO: 311 - protein, 841 aa, synthetic construct
  REGION 1..841: note = SLR protein
MEEENIVNGDRPRDLVFPGTAGLQLYQSLYKYSYITDGIIDAHTNEVISYAQIFETSCRLAVSLEKYGLDHNNVVAICSENNIHFFGPLIAALYQGIPMATSNDMYTEREMIGHLNISKPCLMFCSKKSLPFILKVQKHLDFLKKVIVIDSMYDINGVECVFSFVSRYTDHAFDPVKFNPKEFDPLERTALIMTSSGTTGLPKGVVISHRSITIRFVHSSDPIYGTRIAPDTSILAIAPFHHAFGLFTALAYFPVGLKIVMVKKFEGEFFLKTIQNYKIASIVVPPPIMVYLAKSPLVDEYNLSSLTEIACGGSPLGRDIADKVAKRLKVHGILQGYGLTETCSALILSPNDRELKKGAIGTPMPYVQVKVIDINTGKALGPREKGEICFKSQMLMKGYHNNPQATRDALDKDGWLHTGDLGYYDEDRFIYVVDRLKELIKYKGYQVAPAELENLLLQHPNISDAGVIGIPDEFAGQLPSACVVLEPGKTMTEKEVQDYIAELVTTTKHLRGGVVFIDSIPKGPTGKLMRNELRAIFAREQAKSKLRAKRSGSGATNFSLLKQAGDVEENPGPMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 312 - DNA, 2526 bp, synthetic construct
  misc_feature 1..2526: note = Polynucleotide coding SLR protein
  source 1..2526: mol_type = other DNA
atggaagaagagaacatcgtgaatggcgatcgccctcgggatctggtgttccctggcacagccggcctgcagctgtatcagtccctgtataaatactcttacatcaccgacggaatcatcgacgcccacaccaacgaggtgatctcctatgcccagattttcgaaacaagttgccgcctggccgtgagcctggagaagtatggcctggatcacaacaacgtggtggccatttgcagcgagaacaacatccacttcttcggccctctgatcgctgccctataccaggggattccaatggccacatccaacgatatgtacaccgagagggagatgatcggccacctgaacatctccaagccatgtctgatgttctgttccaagaagtccctgccattcatcctgaaggtgcagaagcacctggactttctcaagaaggtgatcgtgatcgacagcatgtacgacatcaacggcgtggagtgcgtgttcagtttcgtgtcccggtacaccgatcatgcgttcgatccagtgaagttcaaccctaaagagtttgatcccctggagagaaccgcgctgatcatgacatcctctggaacaaccggcctgcctaagggcgtggtgatcagccacaggagcatcaccatcagattcgtccacagcagcgatcccatctacggcacccgcatcgccccagatacatccatcctggccatcgcccctttccaccacgccttcggactgtttaccgccctggcttactttccagtgggcctgaagatcgtgatggtgaaaaagtttgagggcgagttcttcctgaagaccatccagaactacaagatcgcttctatcgtggtgcctcctccaatcatggtgtatctggccaagagccctctggtggatgagtacaatctgtccagcctgacagagatcgcctgtggcggctcccctctgggcagagacatcgccgacaaggtggccaagagactgaaggtccacggcatcctgcagggctatggcctgaccgagacctgtagcgccctgatcctgagccccaacgatagagagctgaagaagggcgccatcggcacccctatgccctatgtccaggtgaaggtgattgacatcaacaccggcaaagccctgggaccaagagagaagggcgagatttgcttcaagagccagatgctgatgaagggctaccacaacaacccacaggccaccagggatgccctggacaaggacgggtggctgcacaccggcgatctgggctactacgacgaggacagattcatctatgtggtggatcggctgaaagaactcatcaagtacaagggctaccaggtggcccctgccgagctggagaacttgcttctgcagcaccctaacatctctgatgccggcgtcatcggcatcccagacgagtttgccggccagctgccttccgcctgtgtcgtgctggagcctggcaagaccatgaccgagaaggaggtgcaggattatatcgccgagctggtgaccaccaccaagcacctgcggggcggcgtggtgttcatcgacagcattccgaaaggcccaacaggcaagctgatgagaaacgagctgagggccatctttgcccgcgagcaggccaagtccaagctgagggccaagcggtccggatccggagccaccaacttcagcctgctgaagcaggccggcgacgtggaggagaaccccggccccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctga

SEQ ID NO: 313-314 - skipped (2 entries)

SEQ ID NO: 315 - protein, 9 aa, synthetic construct
  REGION 1..9: note = CDRL1 of anti-NY-ESO1 157-165 antibody
  VARIANT 1..2: note = X can be any naturally occurring amino acid
  VARIANT 6: note = X can be any naturally occurring amino acid
  VARIANT 8..9: note = X can be any naturally occurring amino acid
XXDVGXYXX

SEQ ID NO: 316 - skipped

SEQ ID NO: 317 - protein, 9 aa, synthetic construct
  REGION 1..9: note = CDRL1 of anti-CD19 antibody
  VARIANT 1: note = X can be any naturally occurring amino acid
  VARIANT 3..4: note = X can be any naturally occurring amino acid
  VARIANT 6..7: note = X can be any naturally occurring amino acid
XSXXGXXNY

SEQ ID NO: 318-320 - skipped (3 entries)

SEQ ID NO: 321 - protein, 12 aa, synthetic construct
  REGION 1..12: note = CDRL3 of anti-CD19 antibody
  VARIANT 3..9: note = X can be any naturally occurring amino acid
  VARIANT 11: note = X can be any naturally occurring amino acid
QQXXXXXXXLXT

SEQ ID NO: 322 - skipped

SEQ ID NO: 323 - protein, 9 aa, synthetic construct
  REGION 1..9: note = CDRH2 of anti-NY-ESO1 157-165 antibody
  VARIANT 1..4: note = X can be any naturally occurring amino acid
  VARIANT 6: note = X can be any naturally occurring amino acid
XXXXSXGST

SEQ ID NO: 324 - protein, 10 aa, synthetic construct
  REGION 1..10: note = CDRH3 of anti-NY-ESO1 157-165 antibody
  VARIANT 2..6: note = X can be any naturally occurring amino acid
AXXXXXGMDV

(19) United States
(12) Patent Application Publication
(10) Pub. No.: US 2025/0236864 A1
(43) Pub. Date: Jul. 24, 2025

(54) CAR LIBRARY AND scFv MANUFACTURING METHOD

(71) Applicant: NATIONAL UNIVERSITY CORPORATION EHIME UNIVERSITY, Matsuyama-shi (JP)

(72) Inventors: Toshiki OCHI, Ehime (JP); Masaki YASUKAWA, Ehime (JP); Hiroshi FUJIWARA, Ehime (JP); Katsuto TAKENAKA, Ehime (JP)

(21) Appl. No.: 19/035,459

(22) Filed: Jan. 23, 2025

Related U.S. Application Data
(63) Continuation of application No. 17/428,214, filed on Aug. 3, 2021, now Pat. No. 12,241,061.

(30) Foreign Application Priority Data
Feb. 4, 2019 (JP) 2019-018269
May 18, 2019 (JP) 2019-094188
Jul. 30, 2019 (JP) 2019-140121
Oct. 4, 2019 (JP) 2019-184071

(51) Int. Cl. C12N 15/10; A61K 40/11; A61K 40/31; A61K 40/42; C07K 14/705; C07K 14/725; C07K 16/28; C12N 5/0783

(52) U.S. Cl. CPC C12N 15/1068; A61K 40/11; A61K 40/31; A61K 40/4211; A61K 40/4269; C07K 14/7051; C07K 14/70521; C07K 16/2803; C12N 5/0636; A61K 2239/31; A61K 2239/38; A61K 2239/48; C07K 2317/14; C07K 2317/565; C07K 2317/622; C07K 2319/02; C07K 2319/03; C07K 2319/30; C07K 2319/33

(57) ABSTRACT

Provided are a CAR library used to screen scFvs that can be functional in CAR-T cells, and an scFv manufacturing method in which the CAR library is used. A chimeric antigen receptor (CAR) library of the present invention includes nucleic acids coding for first CARs. Each of the first CARs includes a first antigen-binding domain, a first transmembrane domain, and a first intracellular signaling domain. The first antigen-binding domain includes a first single-chain antibody (scFv) to be screened for the ability to bind to a target antigen. The first scFv includes a first heavy-chain variable region and a first light-chain variable region. The first heavy-chain variable region and the first light-chain variable region meet a predetermined condition.

### SEQUENCE LISTING

The present application is being filed along with a Sequence Listing in electronic format. The Sequence Listing is provided as a file entitled Sequence_Listing_0014USC1-2.xml created on Apr. 14, 2025, which is 414 KB in size. The information in the electronic format of the Sequence Listing is incorporated herein by reference in its entirety.

### TECHNICAL FIELD

The present invention relates to a CAR library and an scFv manufacturing method.

### BACKGROUND ART

Attempts have been made to treat cancers using T cells (chimeric antigen receptor-expressing T cells, CAR-T cells) expressing single-chain antibodies (single-chain Fvs: scFvs) capable of binding to cancer-specific antigens or target antigens.

scFvs are screened using a technique in which hybridomas derived from B cells isolated from living organisms are used, or a phage display technique. Specifically, in the former technique, a hybridoma library is produced using B cells isolated from living organisms after antigen immunization. Then, a hybridoma that produces an antibody capable of binding to a target antigen is selected, and the sequence of the antigen-recognition site of the antibody produced by the hybridoma is determined.

In the latter technique, an scFv library that includes polynucleotides coding for scFvs is produced, and the scFvs are expressed in phages. Then, a phage capable of binding to a target antigen is selected, and the polynucleotide included in the phage is identified.

### SUMMARY OF INVENTION

### Technical Problem

With both of the techniques, an scFv capable of binding to a target antigen can be selected, but it is not clear whether this scFv is also functional in CAR-T cells. Accordingly, after the above-mentioned selection, it is necessary to express target antigen-binding scFvs in T cells and examine whether they are functional.

To address this, the present invention provides a CAR library used to screen scFvs that can be functional in CAR-T cells, for example, and an scFv manufacturing method in which the CAR library is used.

### Solution to Problem

To achieve the above-mentioned object, a chimeric antigen receptor (CAR) library of the present invention includes nucleic acids coding for first chimeric antigen receptors (CARs),
wherein each of the first CARs includes a first antigen-binding domain, a first transmembrane domain, and a first intracellular signaling domain,
the first antigen-binding domain includes a first single-chain antibody (scFv) to be screened for the ability to bind to a target antigen,
the first scFv includes a first heavy-chain variable region and a first light-chain variable region,
the first heavy-chain variable region and the first light-chain variable region meet Condition 1 or Condition 2 below,

Condition 1 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of an antibody capable of binding to the target antigen or an antigen-binding fragment of the antibody, respectively, and
a light-chain complementarity determining region (CDRL) 1, a CDRL2, and a CDRL3 in the first light-chain variable region include a CDRL1, a CDRL2, and a CDRL3 in a light-chain variable region of a first B cell receptor, respectively, and

Condition 2 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of a first B cell receptor, respectively, and
a light-chain complementarity determining region (CDRL) 1, a CDRL2, and a CDRL3 in the first light-chain variable region include a CDRL1, a CDRL2, and a CDRL3 in a light-chain variable region of an antibody capable of binding to the target antigen or an antigen-binding fragment of the antibody, respectively.

An scFv manufacturing method of the present invention (also referred to as a “first screening method” hereinafter) includes
a first expression step of expressing the CAR library of the present invention in immune cells;
a first contact step of bringing the immune cells obtained in the first expression step into contact with the target antigen; and
a first selection step of selecting the first scFvs of the CARs expressed in the immune cells that have bound to the target antigen in the first contact step as first candidate scFvs capable of binding to the target antigen.

### Advantageous Effects of Invention

With the present invention, it is possible to provide a CAR library used to screen scFvs that can be functional in CAR-T cells, for example, and an scFv manufacturing method in which the CAR library is used.

### BRIEF DESCRIPTION OF DRAWINGS

FIG. 1 shows dot plots showing the results of flow cytometry in Example 1.

FIG. 2 shows dot plots showing the results of flow cytometry in Example 1.

FIG. 3 is a graph indicating the ratios of A2/NY-ESO-1₁₅₇ tetramer-positive cells in Example 1.

FIG. 4 is a graph indicating changes in a CD69 expression level in Example 1.

FIG. 5 is a graph indicating changes in a CD69 expression level in Example 1.

FIG. 6 shows dot plots showing the results of flow cytometry in Example 1.

FIG. 7(A) shows dot plots showing the results of flow cytometry in Example 1.

FIG. 7(B) shows dot plots showing the results of flow cytometry in Example 1.

FIG. 8 shows dot plots showing the results of flow cytometry in Example 2.

FIG. 9 shows dot plots showing the results of flow cytometry in Example 2.

FIG. 10 is a graph indicating the ratios of A2/NY-ESO-1₁₅₇ tetramer-positive cells in Example 2.

FIG. 11 is a graph indicating changes in a CD69 expression level in the case where the peptide concentration is 10 μg/mL in Example 2.

FIG. 12 is a graph indicating changes in a CD69 expression level in Example 2.

FIG. 13 shows dot plots showing the results of flow cytometry in Example 3.

FIG. 14 shows dot plots showing the results of flow cytometry in Example 4.

FIG. 15(A) and FIG. 15(B) show graphs indicating changes in a cytokine production amount in Example 4. FIG. 15(A) shows the results from CD8-positive T cells, and FIG. 15(B) shows the results from CD4-positive T cells.

FIG. 16(A) and FIG. 16(B) show graphs indicating changes in a cytokine production amount in Example 5. FIG. 16(A) shows the results from T cells expressing a 3M4E5LH CAR, and FIG. 16(B) shows the results from T cells expressing an L1-3M4E5H CAR.

FIG. 17 shows dot plots showing the results of flow cytometry in Example 6.

FIG. 18(A) and FIG. 18(B) show graphs indicating changes in a cytokine production amount in Example 6. FIG. 18(A) shows the results from CD8-positive T cells, and FIG. 18(B) shows the results from CD4-positive T cells.

FIG. 19(A) and FIG. 19(B) show graphs indicating changes in a cytokine production amount in Example 6. FIG. 19(A) shows the results from CD8-positive T cells, and FIG. 19(B) shows the results from CD4-positive T cells.

FIG. 20(A) and FIG. 20(B) show graphs indicating changes in a cytokine production amount in Example 7. FIG. 20(A) shows the results from T cells expressing a 3M4E5LH CAR, and FIG. 20(B) shows the results from T cells expressing a 3M4E5H-L1 CAR.

FIG. 21(A) and FIG. 21(B) show graphs indicating changes in a cytokine production amount in Example 7. FIG. 21(A) shows the results from CD8-positive T cells, and FIG. 21(B) shows the results from CD4-positive T cells.

FIG. 22 shows dot plots showing the results of flow cytometry in Example 8.

FIG. 23(A) and FIG. 23(B) show graphs indicating changes in a cytokine production amount in Example 8. FIG. 23(A) shows the results from CD8-positive T cells, and FIG. 23(B) shows the results from CD4-positive T cells.

FIG. 24(A) and FIG. 24(B) show graphs indicating changes in a cytokine production amount in Example 8. FIG. 24(A) shows the results from CD8-positive T cells, and FIG. 24(B) shows the results from CD4-positive T cells.

FIG. 25 shows dot plots showing the results of flow cytometry in Example 9.

FIG. 26 is graphs indicating changes in a CD69 expression level in Example 9.

FIG. 27 shows dot plots showing the results of flow cytometry in Example 10.

FIG. 28 shows dot plots showing the results of flow cytometry in Example 10.

FIG. 29 shows dot plots showing the results of flow cytometry in Example 10.

FIG. 30 shows graphs indicating changes in a CD69 expression level in Example 10.

FIG. 31 shows graphs indicating relative ratios of CD19 dimer-positive cells in Example 10.

FIG. 32 shows graphs indicating relative values of CD69 expression levels when the amount of target cells is varied in Example 10.

FIG. 33 shows graphs indicating ratios of cytokine-producing cells in Example 10.

FIG. 34(A) shows a protocol relating to anti-tumor activity in living organisms in Example 11.

FIG. 34(B) shows photographs using an image analyzer.

FIG. 34(C) shows a graph indicating the sizes of tumors.

FIG. 35 is a graph indicating a relationship between the structural avidity index value and the functional avidity index value in Example 12.

FIG. 36 shows graphs indicating cytotoxicity in Example 13.

FIG. 37(A) shows a protocol relating to the overall survival rate in living organisms in Example 14.

FIG. 37(B) shows a graph indicating the Kaplan-Meier curves showing the overall survival.

FIG. 38(A) shows a protocol relating to the overall survival rate in living organisms in Example 14.

FIG. 38(B) shows a graph indicating the Kaplan-Meier curves showing the overall survival.

FIG. 39 shows graphs indicating the cell proliferation rates in Example 15.

FIG. 40 shows graphs indicating the ratios of cytokine-producing cells in Example 15.

FIG. 41 shows graphs indicating the cytotoxic activity in Example 15.

FIG. 42 shows dot plots indicating the distributions of CD45RA/CD62L-positive cells in CD8-positive CAR-T cells and CD4-positive CAR-T cells before and after coculture in Example 15.

FIG. 43 shows graphs indicating the CAR-T cell division ratios, the ratios of CD45RA-positive CD62L-positive CCR7-positive cells before and after coculture, the ratios of CD45RA-negative CD62L-positive CCR7-positive cells before and after coculture, and the ratios of CD45RA-positive CD62L-negative PD1-positive cells before and after coculture, in Example 15.

FIG. 44 is a diagram for illustrating a mechanism in which scFvs having an excellent anti-tumor effect in vivo can be obtained using the screening method of the present invention in Example 15.

### DESCRIPTION OF EMBODIMENTS

### CAR Library

As described above, the chimeric antigen receptors (CAR) library of the present invention includes nucleic acids coding for first CARs, wherein each of the first CARs includes a first antigen-binding domain, a first transmembrane domain, and a first intracellular signaling domain, the first antigen-binding domain includes a first single-chain antibody (scFv) to be screened for the ability to bind to a target antigen, the first scFv includes a first heavy-chain variable region and a first light-chain variable region, and the first heavy-chain variable region and the first light-chain variable region meet Condition 1 or Condition 2 below. The CAR library of the present invention is characterized by meeting Condition 1 or Condition 2 below, and there is no particular limitation on other configurations and conditions.

### Condition 1:

The heavy-chain complementarity determining region (CDRH) 1, the CDRH2, and the CDRH3 in the first heavy-chain variable region include the CDRH1, the CDRH2, and the CDRH3 in the heavy-chain variable region of an antibody capable of binding to the target antigen or an antigen-binding fragment of the antibody (also referred to as an “antibody or the like” hereinafter), respectively, and
the light-chain complementarity determining region (CDRL) 1, the CDRL2, and the CDRL3 in the first light-chain variable region include the CDRL1, the CDRL2, and the CDRL3 in the light-chain variable region of a first B cell receptor, respectively.

### Condition 2:

The heavy-chain complementarity determining region (CDRH) 1, the CDRH2, and the CDRH3 in the first heavy-chain variable region include the CDRH1, the CDRH2, and the CDRH3 in the heavy-chain variable region of a first B cell receptor, respectively, and
the light-chain complementarity determining region (CDRL) 1, the CDRL2, and the CDRL3 in the first light-chain variable region include the CDRL1, the CDRL2, and the CDRL3 in the light-chain variable region of an antibody capable of binding to the target antigen or an antigen-binding fragment of the antibody, respectively.

The CAR library of the present invention is characterized in that each of the first CARs includes the first antigen-binding domain, the first transmembrane domain, and the first intracellular signaling domain, and the first heavy-chain variable region and the first light-chain variable region meet Condition 1 or Condition 2 above, and there is no particular limitation on other configurations and conditions.

In the CAR library of the present invention, each of the first CARs is a protein that includes the first antigen-binding domain, the first transmembrane domain, and the first intracellular signaling domain. The first CAR has a structure similar to that of a CAR that includes an extracellular domain capable of binding to an antigen, a transmembrane domain, and an intracellular signaling domain, for example, and the extracellular domain capable of binding to an antigen is changed to a first antigen-binding domain to be screened for the ability to bind to the target antigen. In the CAR library of the present invention, the first CAR has a structure similar to that of the CAR as mentioned above. Accordingly, a first CAR capable of binding to the target antigen can induce a signal via the first intracellular signaling domain in the expressing cell, for example. Thus, a first CAR capable of binding to the target antigen can activate the expressing cell (e.g., activating the cell proliferation, the expression of an activation marker, and the like), for example. That is, with the CAR library of the present invention, a first screening method, which will be described later, can be used to increase the ratio of cells expressing a first CAR capable of binding to the target antigen in a cell group expressing the CAR library, for example. Therefore, with the CAR library of the present invention, scFvs capable of binding to the target antigen can be screened using a smaller number of, or a smaller number of types of, nucleic acids compared with a phage display technique, for example. Moreover, a first CAR capable of binding to the target antigen can activate the expressing cell in the first screening method, which will be described later, for example, and thus it can be said that the first CAR includes an scFv that is functional in CAR-T cells. Therefore, with the CAR library of the present invention, scFvs that can be functional in CAR-T cells can be screened, for example.

Moreover, in the CAR library of the present invention, the first antigen-binding domain includes the first single-chain antibody (scFv) to be screened for the ability to bind to the target antigen. The first scFv has a structure similar to that of an scFv that is a single-chain polypeptide derived from an antibody capable of binding to an antigen and has an ability to bind to the antigen, for example. The scFv is a polypeptide obtained by coupling the fragments (Fvs) of variable regions of the heavy chain (H chain) and the light chain (L chain) of the antibody capable of binding to the antigen. On the other hand, in the first scFv, either the heavy-chain variable region or the light-chain variable region is a variable region of the antibody or the like capable of binding to the target antigen, and the other one is a variable region derived from a B cell receptor, namely a variable region that may or may not bind to the target antigen, as described later, for example. With the CAR library of the present invention in which the first scFv has such a configuration, heavy-chain variable regions or light-chain variable regions capable of binding to the target antigen can be screened, for example. Therefore, with the CAR library of the present invention, scFvs capable of binding to the target antigen can be screened using a smaller number of, or a smaller number of types of, nucleic acids compared with a phage display technique in which heavy-chain variable regions and light-chain variable regions capable of binding to the target antigen are screened at a time, for example.

In the CAR library of the present invention, the nucleic acids coding for first CARs are nucleic acids (polynucleotides) coding for the amino acid sequences of the first CARs, for example. The nucleic acids may be constituted by DNA, RNA, or both. The nucleic acids may be constituted by natural nucleic acids, modified nucleic acids obtained by modifying natural nucleic acids, artificial nucleic acids such as ENAs (2′-O,4′-C-Ethylene-bridged Nucleic Acids), LNAs (Locked nucleic acids), PNAs (Peptide Nucleic Acids), or morpholinos, or two or more of these compounds.

The term “domain” as used in the present invention means a region of a polypeptide specified based on the three-dimensional structure or functionality, for example. The polypeptide may be a peptide containing 10 or more amino acids, for example.

There is no particular limitation on the target antigen of the first scFv, and any antigen can be used. Examples of the target antigen include tumor antigens, viral antigens, bacterial antigens, parasitic antigens, antigens linked to autoimmune diseases, and sugar chain antigens. The tumor antigens are biomolecules having antigenicity such as antigens that are newly expressed or are expressed at an increased level due to cell canceration, for example. The tumor antigens may be tumor-specific antigens or tumor-related antigens, for example.

Examples of the tumor antigens include 5T4, α5β1-integrin, activated integrin β7, 707-AP, α-fetoprotein (AFP), lectin-reactive AFP, ART-4, AURKA (AURORA A), B7H4, BAGE, β-catenin, BCMA, Bcr-abl, BTAA, MN/CA IX antigens, CA125, CA19-9, CA72-4, CAMEL, CAP-1, CASP-8, CD4, CD19, CD20, CD22, CD25, CD27, CD30, CD33, CD47, CD52, CD56, CD80, CD96, CD123, CDK4, carcinoembryonic antigens (CEAs), CLL1, CT, cyclin A1, Cyp-B, DAM, EGFR, ErbB3, ELF2M, EMMPRIN, EpCam, ETV6-AML1, G250, GAGE (GAGE-1, GAGE-2, etc.), GD2 (ganglioside G2), GnT-V, Gp100, HAGE, β-human chorionic gonadotropin (HCG), HER2/neu, HLA-A*0201-R170I, HPV-E7, HSP70-2M, HST-2, iCE, insulin growth factor (IGF)-1, IGF-2, IGF-1R, IL-2R, IL-5, KIAA0205, K-Ras, LAGE, LDLR/FUT, MAGE (MAGE-3, MAGE-4, MAGE-5, MAGE-6, etc.), MART-1/melan-A, MART-2/Ski, MC1R, mesothelin (MSLN), myosin, MUC1, MUM-1, MUM-2, MUM-3, NA88-A, prostatic acid phosphatase (PAP), proteinase-3, PRAME (melanoma antigen preferentially expressed in tumors), p53, p190 minor bcr-abl, Pml/RARα, prostatic tumor antigen-1 (PCTA-1), PRAME, prostate-specific antigen (PSA), PSM, PSMA, RAGE, RAS, RHAMM (CD168), RU1, RU2, SAGE, SART-1, SART-3, thyroglobulin, survivin, telomerase reverse transcriptase (TERT or TRT), TEL/AML1, TGFβ, TIM3, TPI/m, TRP-1, TRP-2, TRP-2/INT2, VEGF, WT1, NY-Eso-1, and NY-Eso-B.

Examples of the viral antigens include antigens derived from viruses belonging to: the family Adenoviridae including adenoviruses; the family Coronaviridae including coronaviruses; the family Filoviridae including the genus Ebolavirus; the family Flaviviridae including hepatitis C virus (HCV), Dengue virus, Japanese encephalitis virus, west Nile virus, and yellow fever virus; the family Hepadnaviridae including hepatitis B virus (HVB); the family Herpesviridae including herpes simplex virus-1 (HSV-1), herpes simplex virus-2 (HSV-2), varicella zoster virus (VZV), human cytomegalovirus (HCMV), Epstein-Barr virus (EBV), and Kaposi's sarcoma-associated herpesvirus (KSHV); the family Orthomyxoviridae including the genus Influenzavirus A, the genus Influenzavirus B, and the genus Influenzavirus C; the family Paramyxoviridae including measles virus, Human parainfluenza viruses 1 to 4, Mumps virus, and Respiratory syncytial virus; the family Parvoviridae including Parvovirus B19; the family Picornaviridae including enteroviruses, poliovirus, human rhinoviruses A and B, hepatitis A virus, coxsackievirus, and echo virus; the family Poxviridae including variola virus and Vaccinia virus; the family Retroviridae including human immunodeficiency viruses (HIVs)-1 and 2 and human T-lymphocytropic viruses (HTLV)-I and II; the family Rhabdoviridae including Rabies virus and Vesicular stomatitis virus; and the family Togaviridae including Rubella virus and Chikungunya virus.

Examples of the bacterial antigens include antigens derived from bacteria belonging to: the genus Clostridium including Clostridium tetani; the genus Escherichia including Escherichia coli; the genus Helicobacter including Helicobacter pyloris; the genus Legionella including Legionella pneumophila; the genus Listeria including Listeria monocytogenes; the genus Mycobacterium including Mycobacterium tuberculosis, Mycobacterium leprae, Mycobacterium avium, Mycobacterium intracellulare, Mycobacterium kansasii, and Mycobacterium gordonae; the genus Neisseria including Neisseria gonorrhoeae and Neisseria meningitidis; the genus Pseudomonas including Pseudomonas aeruginosa; the genus Salmonella including Salmonella enterica serovar Typhi and Salmonella enterica serovar Paratyphi A; the genus Staphylococcus including Staphylococcus aureus; and the genus Streptococcus including Streptococcus pneumoniae and Streptococcus pyogenes.

Examples of the parasitic antigens include antigens derived from parasites such as Clonorchis sinensis, Schistosoma japonicum, Ascaris lumbricoides, Enterobius vermicularis, Cysticercus cellulosae, Diphyllobothrium latum, Echinococcus, Entamoeba histolytica, and Plasmodium.

In the CAR library of the present invention, the first scFv includes the first heavy-chain variable region and the first light-chain variable region. The first heavy-chain variable region and the first light-chain variable region have structures similar to those of the heavy-chain variable region and the light-chain variable region in an antibody molecule, respectively. In general, the heavy-chain variable region and the light-chain variable region in an antibody molecule each include three complementarity determining regions (CDRs). The CDRs are also referred to as “hypervariable domains”. The CDRs are regions in which the primary structure is particularly likely to be variable in the variable regions of the heavy chain and the light chain, and the primary structure generally includes three CDRs. In the present invention, the three CDRs in the heavy-chain variable region are referred to as a heavy-chain CDR 1 (CDRH1), a heavy-chain CDR 2 (CDRH2), and a heavy-chain CDR 3 (CDRH3), in this order from the amino terminus (N terminus) of the amino acid sequence of the heavy-chain variable region, and the three CDRs in the light-chain variable region are referred to as a light-chain CDR 1 (CDRL1), a light-chain CDR 2 (CDRL2), and a light-chain CDR 3 (CDRL3), in this order from the amino terminus of the amino acid sequence of the light-chain variable region. These sites are close to one another in the three-dimensional structure and determine the binding specificity for an antigen.

The first heavy-chain variable region includes the CDRH1, the CDRH2, and the CDRH3. The first light-chain variable region includes the CDRL1, the CDRL2, and the CDRL3. The first heavy-chain variable region and the first light-chain variable region meet Condition 1 or Condition 2 above.

In Condition 1 above, the CDRH1, the CDRH2, and the CDRH3 of an antibody or the like capable of binding to the target antigen are used as those in the first heavy-chain variable region, and the CDRL1, the CDRL2, and the CDRL3 in the first light-chain variable region are screened for the ability to bind to the target antigen. For example, the descriptions of an antibody and an antigen-binding fragment regarding the antibody of the present invention or the like, which will be described later, can be applied to specific examples of the antibody or the antigen-binding fragment thereof.

Known antibodies or the like can be used as the antibody or the like capable of binding to the target antigen in accordance with the target antigen, for example. As a specific example, a 3M4E5 antibody can be used as an antibody against a complex of HLA-A*02:01 and NY-ESO-1₁₅₇₋₁₆₅, for example. An FMC63 antibody can be used as an antibody against human CD19, for example. The antibody or the like capable of binding to the target antigen may be an scFv obtained using a first screening method of the present invention, which will be described later, or an antibody obtained through immunization with the target antigen, for example. The CDRH1, the CDRH2, and the CDRH3 in the first heavy-chain variable region may respectively be polypeptides that consist of the amino acid sequences of the CDRH1, the CDRH2, and the CDRH3 of the antibody or the like capable of binding to the target antigen, or polypeptides that include the amino acid sequences of the CDRH1, the CDRH2, and the CDRH3 of the antibody or the like capable of binding to the target antigen, for example.

Regions in the first heavy-chain variable region other than the CDRH1, the CDRH2, and the CDRH3, namely framework regions (FRs), may include the FRs in the heavy-chain variable region of the antibody or the like capable of binding to the target antigen, for example. The number of the FRs above in the primary structure is generally four. In the present invention, the four FRs in the heavy-chain variable region are referred to as a heavy-chain FR 1 (FRH1), a heavy-chain FR 2 (FRH2), a heavy-chain FR 3 (FRH3), and a heavy-chain FR 4 (FRH4), in this order from the N terminus of the amino acid sequence of the heavy-chain variable region. It should be noted that the CDRHs and the FRHs are arranged such that the FRH1, the CDRH1, the FRH2, the CDRH2, the FRH3, the CDRH3, and the FRH4 are lined up in this order from the N terminus of the amino acid sequence of the heavy-chain variable region, for example. The FRH1, the FRH2, the FRH3, and the FRH4 in the first heavy-chain variable region may respectively be polypeptides that consist of the amino acid sequences of the FRH1, the FRH2, the FRH3, and the FRH4 of the antibody or the like capable of binding to the target antigen, or polypeptides that include the amino acid sequences of the FRH1, the FRH2, the FRH3, and the FRH4 of the antibody or the like capable of binding to the target antigen, for example. It is preferable that “the antibody or the like capable of binding to the target antigen” in the descriptions of the CDRHs and “the antibody or the like capable of binding to the target antigen” in the descriptions of the FRHs are the same antibody or the like.

The first heavy-chain variable region may include the heavy-chain variable region of the antibody or the like capable of binding to the target antigen, for example. In this case, the first heavy-chain variable region may be a polypeptide that consist of the amino acid sequence of the heavy-chain variable region of the antibody or the like capable of binding to the target antigen, or a polypeptide that include the amino acid sequence of the heavy-chain variable region of the antibody or the like capable of binding to the target antigen, for example.

The first light-chain variable region is encoded by a VJ gene fragment formed through VJ gene recombination of a V gene fragment and a J gene fragment, for example. Therefore, the first B cell receptor may be a B cell receptor that includes a polypeptide encoded by an artificial VJ gene fragment designed as an artificial combination of a V gene fragment and a J gene fragment, for example. B cells in a living organism express a light-chain variable region encoded by a product formed through VJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the CAR library of the present invention, the first B cell receptor is preferably the light-chain variable region of the B cell receptor derived from isolated B cells for the reason that the CAR library can be more easily prepared.

The CDRL1, the CDRL2, and the CDRL3 in the first light-chain variable region may respectively be polypeptides that consist of the amino acid sequences of the CDRL1, the CDRL2, and the CDRL3 of the first B cell receptor, or polypeptides that include the amino acid sequences of the CDRL1, the CDRL2, and the CDRL3 of the first B cell receptor, for example.

The FRs in the first light-chain variable region may include the FRs in the light-chain variable region of the first B cell receptor, for example. The number of the FRs above in the primary structure is generally four. In the present invention, the four FRs in the light-chain variable region are referred to as a light-chain FR 1 (FRL1), a light-chain FR 2 (FRL2), a light-chain FR 3 (FRL3), and a light-chain FR 4 (FRL4), in this order from the N terminus of the amino acid sequence of the light-chain variable region. It should be noted that the CDRLs and the FRLs are arranged such that the FRL1, the CDRL1, the FRL2, the CDRL2, the FRL3, the CDRL3, and the FRL4 are lined up in this order from the N terminus of the amino acid sequence of the light-chain variable region, for example. The FRL1, the FRL2, the FRL3, and the FRL4 in the first light-chain variable region may respectively be polypeptides that consist of the amino acid sequences of the FRL1, the FRL2, the FRL3, and the FRL4 of the first B cell receptor, or polypeptides that include the amino acid sequences of the FRL1, the FRL2, the FRL3, and the FRL4 of the first B cell receptor, for example. It is preferable that “the first B cell receptor” in the descriptions of the CDRLs and “the first B cell receptor” in the descriptions of the FRLs are the same B cell receptor.

The first light-chain variable region may include the light-chain variable region of the first B cell receptor, for example. In this case, the first light-chain variable region may be a polypeptide that consists of the amino acid sequence of the light-chain variable region of the first B cell receptor, or a polypeptide that includes the amino acid sequence of the light-chain variable region of the first B cell receptor, for example.

Next, in Condition 2 above, the CDRL1, the CDRL2, and the CDRL3 in the first light-chain variable region are derived from the antibody or the like capable of binding to the target antigen, and the CDRH1, the CDRH2, and the CDRH3 in the first heavy-chain variable region are screened for the ability to bind to the target antigen.

The first heavy-chain variable region is encoded by a VDJ gene fragment formed through VDJ gene recombination of a V gene fragment, a D gene fragment, and a J gene fragment, for example. Therefore, the first B cell receptor may be a B cell receptor that includes a polypeptide encoded by an artificial VDJ gene fragment designed as an artificial combination of a V gene fragment, a D gene fragment, and a J gene fragment, for example. A heavy-chain variable region is expressed in B cells in a living organism after VDJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the CAR library of the present invention, the first B cell receptor is preferably the heavy-chain variable region of the B cell receptor derived from isolated B cells for the reason that the CAR library can be more easily prepared.

The CDRH1, the CDRH2, and the CDRH3 in the first heavy-chain variable region may respectively be polypeptides that consist of the amino acid sequences of the CDRH1, the CDRH2, and the CDRH3 of the first B cell receptor, or polypeptides that include the amino acid sequences of the CDRH1, the CDRH2, and the CDRH3 of the first B cell receptor, for example.

The FRH1, the FRH2, the FRH3, and the FRH4 in the first heavy-chain variable region may respectively be polypeptides that consist of the amino acid sequences of the FRH1, the FRH2, the FRH3, and the FRH4 of the first B cell receptor, or polypeptides that include the amino acid sequences of the FRH1, the FRH2, the FRH3, and the FRH4 of the first B cell receptor, for example. It is preferable that “the first B cell receptor” in the descriptions of the CDRHs and “the first B cell receptor” in the descriptions of the FRHs are the same B cell receptor.

The first heavy-chain variable region may include the heavy-chain variable region of the first B cell receptor, for example. In this case, the first heavy-chain variable region may be a polypeptide that consists of the amino acid sequence of the heavy-chain variable region of the first B cell receptor, or a polypeptide that includes the amino acid sequence of the heavy-chain variable region of the first B cell receptor, for example.

For example, regarding the first light-chain variable region, the descriptions of the antibody or the like capable of binding to the target antigen in Condition 1 above, and the like can be applied to the antibody or the like capable of binding to the target antigen. The CDRL1, the CDRL2, and the CDRL3 in the first light-chain variable region may respectively be polypeptides that consist of the amino acid sequences of the CDRL1, the CDRL2, and the CDRL3 of the antibody or the like capable of binding to the target antigen, or polypeptides that include the amino acid sequences of the CDRL1, the CDRL2, and the CDRL3 of the antibody or the like capable of binding to the target antigen, for example.

The FRs in the first light-chain variable region may include the FRs in the light-chain variable region of the antibody or the like capable of binding to the target antigen, for example. The FRL1, the FRL2, the FRL3, and the FRL4 in the first light-chain variable region may respectively be polypeptides that consist of the amino acid sequences of the FRL1, the FRL2, the FRL3, and the FRL4 of the antibody or the like capable of binding to the target antigen, or polypeptides that include the amino acid sequences of the FRL1, the FRL2, the FRL3, and the FRL4 of the antibody or the like capable of binding to the target antigen, for example. It is preferable that “the antibody or the like capable of binding to the target antigen” in the descriptions of the CDRLs and “the antibody or the like capable of binding to the target antigen” in the descriptions of the FRLs are the same antibody or the like.

The first light-chain variable region may include the light-chain variable region of the antibody or the like capable of binding to the target antigen, for example. In this case, the first light-chain variable region may be a polypeptide that consists of the amino acid sequence of the light-chain variable region of the antibody or the like capable of binding to the target antigen, or a polypeptide that includes the amino acid sequence of the light-chain variable region of the antibody or the like capable of binding to the target antigen, for example.

In the first scFv, the first heavy-chain variable region and the first light-chain variable region are coupled to each other via a linker peptide (Fv linker peptide), for example. It is preferable that the Fv linker peptide does not inhibit the first scFv from binding to the target antigen, for example. The Fv linker peptide is constituted by 1 to 40, 1 to 18, 1 to 15, 1 to 7, 1 to 3, or 1 or 2 amino acids, for example. The Fv linker peptide is constituted by amino acids such as glycine and serine, for example, and a specific example thereof is (GGGGS)ₙ. n in this formula is an integer from 1 to 6, for example. The amino acid sequence of the Fv linker peptide may be the amino acid sequence of the polypeptide represented by Sequence ID No. 1 or 2 below, for example.

Fv Linker Peptide 1
(Sequence ID No. 1)
GSTSGSGKPGSGEGSTKG
Fv Linker Peptide 2
(Sequence ID No. 2)
GGGGSGGGGSGGGGS
Base Sequence Coding for Fv Linker Peptide 1
(Sequence ID No. 3)
5′-GGCTCTACAAGCGGCTCTGGCAAGCCTGGATCTGGCGAGGGAAGCA
CCAAGGGC-3′
Base Sequence Coding for Fv Linker Peptide 2
(Sequence ID No. 4)
5′-GGTGGAGGAGGCTCAGGAGGAGGTGGCTCTGGTGGTGGAGGCTC
G-3′

The first antigen-binding domain includes the first scFv, and the first binding domain may also include a structure other than the scFv that includes the heavy-chain variable region and the light-chain variable region. In a specific example, the first binding domain may be a Fab, Fab′, F(ab′)₂, variable region fragment (Fv), disulfide-bond Fv, or the like.

The first transmembrane domain is a domain that makes up the region that passes through the cell membrane when the first CAR is expressed in a cell, for example. The first transmembrane domain may be the transmembrane domain of a transmembrane protein or an artificial transmembrane domain that has been artificially designed, for example. In the former case, examples of the transmembrane protein include an a chain and a β chain of a T cell receptor, a CD3ζ chain, CD28, CD3ε, CD45, CD4, CD5, CD8 (CD8a or CD8B), CD9, CD16, CD22, CD27, CD33, CD37, CD64, CD80, CD86, CD134, CD137, CD154, ICOS, and GITR, and variants thereof having equivalent functions. The variants having equivalent functions are polypeptides that consist of amino acid sequences with deletion, substitution, insertion, and/or addition of one or several amino acids and function as a transmembrane domain, for example. The term “one or several” above refers to 1 to 15, 1 to 10, 1 to 5, or 1 or 2, for example. In the latter case, the artificial transmembrane domain is a polypeptide that is mainly constituted by hydrophobic amino acids such as leucine, isoleucine, and valine, for example. The artificial transmembrane domain may also include tripeptides constituted by phenylalanine, tryptophan, and valine at the two ends thereof, for example. When the first transmembrane domain is the transmembrane domain of the above-mentioned transmembrane protein, the first transmembrane domain may also include one or several amino acids continuous with the N terminus or C terminus of the transmembrane domain of the transmembrane protein, for example. The example as described above can be applied to the term “one or several” above, for example.

An example of the first transmembrane domain is the transmembrane domain of CD28. The transmembrane domain of CD28 may have an amino acid sequence listed below that corresponds to the amino acid sequence between position 153 and position 179 (Sequence ID No. 5) in the amino acid sequence registered as NCBI Accession No. NP_006130, for example. It should be noted that NCBI Accession No. above is a number assigned to the entire amino acid sequence of the precursor of the protein (the same applies hereinafter). A transmembrane unit that includes the transmembrane domain of CD28 as well as the extracellular region on the N-terminal side of the transmembrane domain and the intracellular region on the C-terminal side of the transmembrane domain may also be used as the transmembrane domain of the CD28, for example.

Transmembrane Domain of CD28
(Sequence ID No. 5)
FWVLVVVGGVLACYSLLVTVAFIIFWV
Base Sequence Coding for Transmembrane Domain of CD28
(Sequence ID No. 6)
5′-TTCTGGGTGCTGGTGGTCGTGGGCGGAGTGCTGGCCTGTTATAGCCTGCTCGTGAC
CGTGGCCTTCATCATCTTTTGGGTG-3′
Transmembrane Unit of CD28
(Sequence ID No. 7)
IEVMYPPPYLDNEKSNGTIIHVKGKHLCPSPLFPGPSKPFWVLVVVGGVLACY
SLLVTVAFIIFWVRSKRSRLLHSDYMNMTPRRPGPTRKHYQPYAPPRDFAAYRS
Base Sequence Coding for Transmembrane Unit of CD28
(Sequence ID No. 8)
5′-ATCGAAGTGATGTACCCCCCTCCCTACCTGGACAACGAGAAGTCCAACGGCACCAT
TATCCACGTGAAGGGAAAGCACCTGTGCCCCAGCCCTCTGTTCCCTGGCCCTAGCA
AGCCTTTCTGGGTGCTGGTGGTCGTGGGCGGAGTGCTGGCCTGTTATAGCCTGCTC
GTGACCGTGGCCTTCATCATCTTTTGGGTGCGCAGCAAGCGGAGCCGGCTGCTGCA
CAGCGACTACATGAACATGACCCCCAGACGGCCCGGACCCACCAGAAAGCACTAC
CAGCCTTACGCCCCTCCCAGAGACTTCGCCGCCTACAGATCT-3′

The first intracellular signaling domain is a domain that can perform intracellular signaling when the first antigen-binding domain in the same protein binds to the target antigen, for example. The first signaling domain may be a domain having an amino acid sequence of a cytoplasmic region of a T cell receptor (TCR) complex or costimulatory molecule, or a variant thereof having functions equivalent to those of such an amino acid sequence, for example. The variant having equivalent functions is a polypeptide that consists of an amino acid sequence with deletion, substitution, insertion, and/or addition of one or several amino acids and functions as an intracellular signaling domain, for example. The descriptions above can be applied to the term “one or several” above, for example.

T cells are activated by an antigen-dependent cytoplasmic signal such as an intracellular signal transmitted via a TCR complex, for example (primary activation). This activation is increased by an antigen-non-specific cytoplasmic signal such as intracellular signal transmitted via a costimulatory molecule (secondary activation). Accordingly, T cells include a domain for activation via the TCR complex (primary-activation domain) and a domain for antigen-non-specific activation (secondary-activation domain), for example. Therefore, the first intracellular signaling domain includes at least either the primary-activation domain or the secondary-activation domain, and preferably both, for example.

The primary-activation domain can adjust primary activation by the TCR complex, for example. An example of a primary-activation domain that induces the primary activation is an intracellular signaling domain that includes an immunoreceptor tyrosine-based activation motif (ITAM). Meanwhile, an example of a primary-activation domain that suppreses the primary activation is an intracellular signaling domain that includes an immunoreceptor tyrosine-based inhibitory motif (ITIM).

Examples of the intracellular signaling domain that includes the ITAM include intracellular signaling domains of CD3ζ, FcRγ, FcRβ, CD3γ, CD3δ, CD3ε, CD5, CD22, CD79a, CD79b, and CD66d, and those of variants thereof having equivalent functions. The variants having equivalent functions are polypeptides that consist of amino acid sequences with deletion, substitution, insertion, and/or addition of one or several amino acids and function as an intracellular signaling domain that includes the ITAM. The descriptions above can be applied to the term “one or several” above, for example. The intracellular signaling domain that includes the ITAM of CD3ζ may have an amino acid sequence that corresponds to the amino acid sequence between position 52 and position 164 in the amino acid sequence registered as NCBI Accession No. NP_932170.1, or the amino acid sequence of Sequence ID No. 9 below, for example. The intracellular signaling domain that includes the ITAM of FcεRIγ may have an amino acid sequence that corresponds to the amino acid sequence between position 45 and position 86 in the amino acid sequence registered as NCBI Accession No. NP_004097.1, for example. The intracellular signaling domain that includes the ITAM of FcεRIβ may have an amino acid sequence that corresponds to the amino acid sequence between position 201 and position 244 in the amino acid sequence registered as NCBI Accession No. NP_000130.1, for example. The intracellular signaling domain that includes the ITAM of CD3γ may have an amino acid sequence that corresponds to the amino acid sequence between position 139 and position 182 in the amino acid sequence registered as NCBI Accession No. NP_000064.1, for example. The intracellular signaling domain that includes the ITAM of CD3δ may have an amino acid sequence that corresponds to the amino acid sequence between position 128 and position 171 in the amino acid sequence registered as NCBI Accession No. NP_000723.1, for example. The intracellular signaling domain that includes the ITAM of CD3ε may have an amino acid sequence that corresponds to the amino acid sequence between position 153 and position 207 in the amino acid sequence registered as NCBI Accession No. NP_000724.1, for example. The intracellular signaling domain that includes the ITAM of CD5 may have an amino acid sequence that corresponds to the amino acid sequence between position 402 and position 495 in the amino acid sequence registered as NCBI Accession No. NP_055022.2, for example. The intracellular signaling domain that includes the ITAM of CD22 may have an amino acid sequence that corresponds to the amino acid sequence between position 707 and position 847 in the amino acid sequence registered as NCBI Accession No. NP_001762.2, for example. The intracellular signaling domain that includes the ITAM of CD79a may have an amino acid sequence that corresponds to the amino acid sequence between position 166 and position 226 in the amino acid sequence registered as NCBI Accession No. NP_001774.1, for example. The intracellular signaling domain that includes the ITAM of CD79b may have an amino acid sequence that corresponds to the amino acid sequence between position 182 and position 229 in the amino acid sequence registered as NCBI Accession No. NP_000617.1, for example. The intracellular signaling domain that includes the ITAM of CD66d may have an amino acid sequence that corresponds to the amino acid sequence between position 177 and position 252 in the amino acid sequence registered as NCBI Accession No. NP_001806.2, for example.

Intracellular Signaling Domain That Includes ITAM of CD3ζ
(Sequence ID No. 9)
RVKSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQA
LPPR
Base Sequence Coding for Intracellular Signaling Domain That
Includes ITAM of CD3ζ
(Sequence ID No. 10)
5′-CGAGTGAAGAGCAGAAGCGCCGACGCCCCTGCCTATCAGCAGGGCCAGAACCAGC
TGTACAACGAGCTGAACCTGGGCAGACGGGAAGAGTACGACGTGCTGGACAAGCG
GAGAGGCAGGGACCCTGAGATGGGCGGCAAGCCCAGAAGAAAGAACCCCCAGGA
AGGCCTGTATAACGAACTGCAGAAAGACAAGATGGCCGAGGCCTACAGCGAGATC
GGCATGAAGGGCGAGCGGAGAAGAGGCAAGGGCCACGATGGCCTGTACCAGGGC
CTGAGCACCGCCACCAAGGACACCTATGACGCCCTGCACATGCAGGCCCTGCCCCC
CAGA-3′

Examples of the secondary-activation domain include intracellular signaling domains of CD2, CD4, CD5, CD8α, CD8β, CD27, CD28, CD134 (OX40), CD137 (4-1BB), CD154, GITR, and ICOS, and those of variants thereof having equivalent functions. The variants having equivalent functions are polypeptides that consist of amino acid sequences with deletion, substitution, insertion, and/or addition of one or several amino acids and function as an intracellular signaling domain. The descriptions above can be applied to the term “one or several” above, for example. Specifically, the intracellular signaling domain of CD2 may have an amino acid sequence that corresponds to the amino acid sequence between position 236 and position 351 in the amino acid sequence registered as NCBI Accession No. NP_001758.2, for example. The intracellular signaling domain of CD4 may have an amino acid sequence that corresponds to the amino acid sequence between position 421 and position 458 in the amino acid sequence registered as NCBI Accession No. NP_000607.1, for example. The intracellular signaling domain of CD5 may have an amino acid sequence that corresponds to the amino acid sequence between position 402 and position 495 in the amino acid sequence registered as NCBI Accession No. NP_055022.2, for example. The intracellular signaling domain of CD8α may have an amino acid sequence that corresponds to the amino acid sequence between position 207 and position 235 in the amino acid sequence registered as NCBI Accession No. NP_001759.3, for example. The intracellular signaling domain of CD8β may have an amino acid sequence that corresponds to the amino acid sequence between position 196 and position 210 in the amino acid sequence registered as NCBI Accession No. AAA35664.1, for example. The intracellular signaling domain of CD27 may have an amino acid sequence that corresponds to the amino acid sequence between position 213 and position 260 in the amino acid sequence registered as NCBI Accession No. M63928.1, for example. The intracellular signaling domain of CD28 may have an amino acid sequence that corresponds to the amino acid sequence between position 181 and position 220 (Sequence ID No. 11) in the amino acid sequence registered as NCBI Accession No. NP_006130.1, for example. The intracellular signaling domain of CD134 may have an amino acid sequence that corresponds to the amino acid sequence between position 241 and position 277 in the amino acid sequence registered as NCBI Accession No. NP_003318.1, for example. The intracellular signaling domain of CD137 may have an amino acid sequence that corresponds to the amino acid sequence between position 214 and position 255 in the amino acid sequence registered as NCBI Accession No. NP_001552.2, for example. The intracellular signaling domain of GITR may have an amino acid sequence that corresponds to the amino acid sequence between position 193 and position 241 in the amino acid sequence registered as NCBI Accession No. NP_004186.1, for example. The intracellular signaling domain of ICOS may have an amino acid sequence that corresponds to the amino acid sequence between position 166 and position 199 in the amino acid sequence registered as NCBI Accession No. NP_036224.1, for example.

Intracellular Signaling Domain of CD28
(Sequence ID No. 11)
RSKRSRLLHSDYMNMTPRRPGPTRKHYQPYAPPRDFAAYRS
Base Sequence Coding for Intracellular Signaling Domain of CD28
(Sequence ID No. 12)
5′-CGCAGCAAGCGGAGCCGGCTGCTGCACAGCGACTACATGAACATGACCCCCAGAC
GGCCCGGACCCACCAGAAAGCACTACCAGCCTTACGCCCCTCCCAGAGACTTCGCC
GCCTACAGATCT-3′

The first CAR includes one or more first intracellular signaling domains, for example. When the first CAR includes a plurality of first intracellular signaling domains, the first intracellular signaling domains may be the same or different. It is preferable that the first intracellular signaling domain includes the intracellular signaling domain that includes the ITAM of CD3ζ as the primary-activation domain, for example. Also, it is preferable that the first intracellular signaling domain includes at least one intracellular signaling domain selected from the group consisting of those of CD27, CD28, and CD137 as the secondary-activation domain, for example. In a specific example, the first intracellular signaling domain includes the intracellular signaling domain that includes the ITAM of CD3ζ and at least one intracellular signaling domain selected from the group consisting of those of CD27, CD28, and CD137, for example. Thus, with the CAR library of the present invention, scFvs capable of binding to the target antigen can be screened using a much smaller number of, or a much smaller number of types of, nucleic acids compared with a phage display technique, in the first screening method of the present invention, which will be described later, for example. When the first intracellular signaling domain includes the intracellular signaling domain that includes the ITAM of CD3ζ and the intracellular signaling domain of CD28, it is preferable that the intracellular signaling domain that includes the ITAM of CD3ζ is arranged on the C-terminal side of the intracellular signaling domain of CD28. When the first intracellular signaling domain includes the intracellular signaling domain that includes the ITAM of CD3ζ and the intracellular signaling domain of CD28, the first intracellular signaling domain may further include at least one of the intracellular signaling domain of CD134 and the intracellular signaling domain of CD137.

The first antigen-binding domain and the first transmembrane domain are directly or indirectly coupled to each other. When the first antigen-binding domain and the first transmembrane domain are indirectly coupled, it is preferable that they are coupled via a spacer peptide. The spacer peptide is constituted by 1 to 300, 1 to 100, 10 to 100, or 25 to 50 amino acids, for example. It is preferable that the spacer peptide includes amino acids (e.g., cysteine, serine, and threonine) that promote the binding of the first CAR to the target antigen and induce and/or increase signal transmission by the first intracellular signaling domain when the first CAR binds to the target antigen. In a specific example, the constant region of immunoglobulin such as the CH1 region or L region, a partial region of CD4, CD8α, CD8β, or CD28, or the like can be used as the spacer peptide, for example. The first spacer peptide has an amino acid sequence that has been artificially designed. An example of the partial region of CD8α is the hinge region of CD8α, and a specific example thereof may have an amino acid sequence that corresponds to the amino acid sequence between position 118 and position 178 in the amino acid sequence registered as NCBI Accession No. NP_001759.3. The partial region of CD8β may have an amino acid sequence that corresponds to the amino acid sequence between position 135 and position 195 in the amino acid sequence registered as NCBI Accession No. AAA35664.1, for example. The partial region of CD4 may have an amino acid sequence that corresponds to the amino acid sequence between position 315 and position 396 in the amino acid sequence registered as NCBI Accession No. NP_000607.1, for example. The partial region of CD28 may have an amino acid sequence that corresponds to the amino acid sequence between position 137 and position 152 (Sequence ID No. 13) in the amino acid sequence registered as NCBI Accession No. NP_006130.1, for example. The spacer peptide may have an amino acid sequence that corresponds to a part of the amino acid sequence of each example.

Partial Region of CD28 (Extracellular Region)
(Sequence ID No. 13)
IEVMYPPPYLDNEKSNGTIIHVKGKHLCPSPLFPGPSKP
Base Sequence Coding for Part of CD28
(Sequence ID No. 14)
5′-ATCGAAGTGATGTACCCCCCTCCCTACCTGGACAACGAGAAGTCCAACGGCACCAT
TATCCACGTGAAGGGAAAGCACCTGTGCCCCAGCCCTCTGTTCCCTGGCCCTAGCA
AGCCT-3′

In the first CAR, the domains may be coupled to each other via a linker peptide (domain linker peptide), for example. The domain linker peptide is constituted by 1 to 40, 1 to 18, 1 to 15, 1 to 7, 1 to 3, or 1 or 2 amino acids, for example. The domain linker peptide is constituted by amino acids such as glycine and serine, for example, and a specific example thereof is (GGGGS)ₙ. n in this formula is an integer from 1 to 6, for example.

An example of the first CAR is a polypeptide that consists of the amino acid sequence (Sequence ID No. 15) represented by Formula (1) below. In Formula (1) below, V₁ and V₂ are the amino acid sequences of the heavy-chain variable region and the light-chain variable region, respectively, or the amino acid sequences of the light-chain variable region and the heavy-chain variable region, respectively. In Formula (1) below, the amino acid sequence between V₁ and V₂ is the amino acid sequence of the Fv linker peptide, and the amino acid sequence located on the C-terminal side of the V₂ includes the amino acid sequences of the spacer peptide, the transmembrane domain, and the intracellular signaling domain. In Formula (1) below, the Fv linker peptide is the Fv linker peptide 1, but may also be the Fv linker peptide 2.

[V₁]-[GSTSGSGKPGSGEGSTKG]-[V₂]-
[IEVMYPPPYLDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRS]-
[RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR]
...(1)

An example of the nucleic acid coding for the first CAR is a polynucleotide consisting of the base sequence (Sequence ID No. 16) represented by Formula (2) below. In Formula (2) below, N₁ and N₂ are the base sequences coding for the heavy-chain variable region and the light-chain variable region, respectively, or the base sequences coding for the light-chain variable region and the heavy-chain variable region, respectively. In Formula (2) below, the base sequence between N₁ and N₂ is the base sequence coding for the Fv linker peptide, and the base sequence located on the 3′-end side of the N₂ includes the base sequences coding for the spacer peptide, the transmembrane domain, and the intracellular signaling domain. In Formula (2) below, the base sequence coding for the Fv linker peptide is the base sequence coding for the Fv linker peptide 1, but may also be the base sequence coding for the Fv linker peptide 2.

(2)
5′-[N₁]-[GGCTCTACAAGCGGCTCTGGCAAGCCTGGATCTGGC
GAGGGAAGCACCAAGGGC]-[N₂]-[ATCGAAGTGATGTACCCCC
CTCCCTACCTGGACAACGAGAAGTCCAACGGCACCATTATCCAC
GTGAAGGGAAAGCACCTGTGCCCCAGCCCTCTGTTCCCTGGCCCT
AGCAAGCCTTTCTGGGTGCTGGTGGTCGTGGGCGGAGTGCTGGCC
TGTTATAGCCTGCTCGTGACCGTGGCCTTCATCATCTTTTGGGTG
CGCAGCAAGCGGAGCCGGCTGCTGCACAGCGACTACATGAACATG
ACCCCCAGACGGCCCGGACCCACCAGAAAGCACTACCAGCCTTAC
GCCCCTCCCAGAGACTTCGCCGCCTACAGATCT]-[CGAGTGAA
GTTCAGCAGAAGCGCCGACGCCCCTGCCTATCAGCAGGGCCAGAA
CCAGCTGTACAACGAGCTGAACCTGGGCAGACGGGAAGAGTACGA
CGTGCTGGACAAGCGGAGAGGCAGGGACCCTGAGATGGGCGGCAA
GCCCAGAAGAAAGAACCCCCAGGAAGGCCTGTATAACGAACTGCA
GAAAGACAAGATGGCCGAGGCCTACAGCGAGATCGGCATGAAGGG
CGAGCGGAGAAGAGGCAAGGGCCACGATGGCCTGTACCAGGGCCT
GAGCACCGCCACCAAGGACACCTATGACGCCCTGCACATGCAGGC
CCTGCCCCCCAGA]-3′.

It is preferable that the CAR library of the present invention includes a plurality of types of nucleic acids, for example. In this case, the CAR library of the present invention is a mixture of a plurality of types of nucleic acids, for example. It is preferable that some or all of the plurality of types of nucleic acids code for different first CARs, and preferably code for different first antigen-binding domains, for example. When the plurality of types of nucleic acids code for different first antigen-binding domains, the regions other than the first antigen-binding domains in the first CARs have the same amino acid sequence or different amino acid sequences, for example. The number of types of nucleic acids included in the CAR library of the present invention is 1×10⁵ to 1×10⁷, 1×10⁵ to 1×10⁶, or 1×10⁶ to 5×10⁶, for example, and preferably about 2×10⁶ (e.g., 1×10⁶ to 3×10⁶). In the phage display technique, the number of types of nucleic acids required to screen scFvs capable of binding to the target antigen is about 1×10⁸. On the other hand, with the CAR library of the present invention, scFvs capable of binding to the target antigen can be screened using as few as about 1×10⁶ types of nucleic acids, for example. Accordingly, with the CAR library of the present invention, scFvs capable of binding to the target antigen can be screened using a smaller number of, or a smaller number of types of, nucleic acids compared with the phage display technique, for example. The number of types of nucleic acids can also be referred to as the heterogeneity of the nucleic acids, for example. The heterogeneity can be measured through restriction enzyme mapping, sequencing of the CDRs and/or the FRHs using the Sanger's method etc., or the like, for example.

The first CAR may include a signal peptide at the N terminus, for example. An example of the signal peptide is a signal peptide serving as an endoplasmic reticulum transport signal. The first CAR may include a tag, for example. Examples of the tag include a peptide tag and a protein tag. Examples of the tag include a FLAG (registered trademark) tag, a HA tag, a His tag, a Myc tag, a V5 tag, and a truncated NGFR (nerve growth factor receptor). The tag peptide is added to at least either the N terminus or the C terminus of the first CAR, for example. When the truncated NGFR is used as the tag, the tag is arranged at the C-terminal side of the first CAR. Thus, with the CAR library of the present invention, the nucleic acid introduction efficiency can be adjusted such that one type of nucleic acid coding for a first CAR is introduced per cell, for example. The first CAR and the tag may be coupled to each other via a linker peptide.

In the present invention, the nucleic acids coding for the first CARs can be prepared based on the amino acid sequences of the first CARs using an ordinary method, for example. In a specific example, the nucleic acids coding for the first CARs can be prepared based on the base sequences coding for the amino acid sequences obtained from the database in which the amino acid sequences of the above-described domains are registered, using a molecular biological technique and/or a chemical synthesis method, for example. The base sequences of the nucleic acids may be subjected to codon optimization in accordance with the source of cells in which the CAR library of the present invention is to be expressed, for example.

The nucleic acids coding for the first CARs may be introduced into expression vectors, for example. The expression vectors can be prepared by coupling the nucleic acids coding for the first CARs to linking vectors, for example. There is no particular limitation on the types of linking vectors, and examples thereof include: retroviral vectors such as oncoretroviral vectors, lentiviral vectors, and pseudo type vectors; and viral vectors such as adenoviral vectors, adeno-associated viral (AAV) vectors, simian viral vectors, vaccinia viral vectors, Sendai viral vectors, Epstein-Barr viral (EBV) vectors, and HSV vectors. Specific examples of the linking vectors include pUC, pCMV, pMX, and pELP. The linking vectors can also be determined as appropriate in accordance with hosts into which the expression vectors are to be introduced, for example. There is no particular limitation on the hosts, and examples thereof include mammalian-derived cultured cells such as CHO cells, Jurkat cells, and Jurkat 76 cells, and immune cells. Examples of the immune cells include lymphocytes, granulocytes, and macrophages. Examples of the lymphocytes include T cells, NK cells, NKT cells, and B cells. The immune cells are cells isolated from a living organism, immune cells induced from stem cells such as multipotent stem cells, or cultured cells derived from immune cells, for example. The T cells may be T cell-like cells. Examples of the T cell-like cells include cultured cells derived from T cells, and specific examples thereof include Jurkat cells and Jurkat 76 cells.

It is preferable that each of the expression vectors includes a regulatory sequence for regulating at least either the expression of the nucleic acid coding for the first CAR or the expression of the first CAR encoded by the nucleic acid coding for the first CAR, for example. Examples of the regulatory sequence include a promoter, a terminator, an enhancer, a polyadenylation signal sequence, and a replication origin sequence (ori). There is no particular limitation on the arrangement of the regulatory sequence in the expression vector. It is sufficient that the regulatory sequence is arranged in the expression vector such that at least either the expression of the nucleic acid coding for the first CAR or the expression of the first CAR encoded by the nucleic acid can be functionally regulated, for example, and the regulatory sequence can be arranged using a known method. For example, a predetermined sequence included in the linking vector may be used as the regulatory sequence, or an additional regulatory sequence may be inserted into the linking vector, or a regulatory sequence included in the linking vector may be replaced with another regulatory sequence.

The expression vector may further include a sequence coding for a selective marker, for example. Examples of the selective marker include drug-resistant markers, fluorescent protein markers, enzymatic markers, and cell-surface receptor markers.

First scFv-Screening Method

A first screening method of the present invention includes: a first expression step of expressing the CAR library of the present invention (also referred to as a “first CAR library” hereinafter) in immune cells; a first contact step of bringing the immune cells obtained in the first expression step into contact with the target antigen; and a first selection step of selecting the first scFvs of the CARs expressed in the immune cells that have bound to the target antigen in the first contact step as first candidate scFvs capable of binding to the target antigen. The first screening method of the present invention is characterized by including the first expression step, the first contact step, and the first selection step, and using the CAR library of the present invention in the first expression step, and there is no particular limitation on the other configurations and conditions. With the first screening method of the present invention, scFvs that can be functional in CAR-T cells can be screened. Moreover, antibodies capable of binding to the target antigen or antigen-binding fragment thereof can be produced based on the amino acid sequences of the CDRs of the heavy-chain variable regions and the light-chain variable regions in the scFvs capable of binding to the target antigen, for example. Accordingly, the first screening method can also be referred to as a method for screening antibodies capable of binding to the target antigen or antigen-binding fragments thereof, for example. The descriptions of the CAR library of the present invention can be applied to the first screening method of the present invention. With the scFv manufacturing method of the present invention, novel scFvs can be screened. Accordingly, the scFv manufacturing method of the present invention can also be referred to as an “scFv screening method”, for example.

When chimeric antigen receptors (CARs) and the like capable of binding to a target antigen are manufactured using a phage display technique or the like, an antibody library in which the number of types of antibodies is about 1×10¹⁰ is produced, and antibodies capable of binding to the target antigen are obtained, followed by preparation of CARs and the like that include scFvs from the antibodies. Then, the effectiveness of the CARs and the like is examined using immune cells or the like. On the other hand, in the first screening method of the present invention, the CAR library of the present invention and the immune cells are used, thus making it possible to simultaneously perform screening of scFvs capable of binding to the target antigen and examination of the effectiveness of the scFvs in the immune cells, for example. Accordingly, with the first screening method of the present invention, scFvs capable of binding to a target antigen and being used for CARs that are functional in immune cells and the like can be more easily screened compared with a phage display technique and the like used to obtain antibodies capable of binding to a target antigen, for example.

In the first expression step, the CAR library of the present invention is expressed in immune cells. Specifically, in the first expression step, the first CAR library is introduced into the immune cells, for example. Then, in the first expression step, the first CAR library is expressed in the immune cells by culturing the immune cells into which the first CAR library has been introduced, for example. There is no particular limitation on a method for introducing the first CAR library, and a known method for introducing nucleic acids into cells can be used, for example. Specific examples of the method for introducing the first CAR library include a method in which a nucleic acid introducing reagent such as a liposome or a cationic lipid is used; and a method in which virus such as retrovirus or lentivirus is used. The immune cells are cultured for 6 hours to 30 days, 6 to 96 hours, or 1 to 30 days, for example, but there is no particular limitation thereto. The immune cells are cultured at a temperature of 28 to 37° C., for example.

The first CAR library may include nucleic acids coding for the first CARs, or expression vectors into which nucleic acids coding for the first CARs have been introduced, for example.

Examples of the immune cells include, but are not particularly limited to, T cells, NK cells, NKT cells, and B cells. Examples of the immune cells include immune cells isolated from a living organism, immune cells induced from stem cells such as multipotent stem cells, and cultured cells derived from immune cells. The isolated immune cells may be immune cell-like cultured cells, for example, and specific examples thereof include T cell-like cultured cells, NK cell-like cultured cells, NKT cell-like cultured cells, and B cell-like cultured cells. The immune cells are immune cells isolated from a living organism, for example, and specific examples thereof include immune cells derived from human peripheral blood. Examples of the T cell-like cultured cells include Jurkat cells and Jurkat 76 cells. The immune cells are preferably T cells, T cell-like cultured cells, NK cells, or NKT cells because scFvs that are more likely to be functional in CAR-T cells can be screened, and immune cells that express first CARs capable of binding to the target antigen can be enriched in the first contact step, which will be described later, for example.

In the first expression step, the number of the immune cells is not particularly limited, and is 1×10⁵ to 1×10⁸, 1×10⁵ to 1×10⁷, 1×10⁵ to 1×10⁶, 1×10⁶ to 5×10⁶, or 1×10⁶ to 3×10⁶, for example.

In the first expression step, it is preferable that the CAR library of the present invention is expressed in the immune cells such that the average number of expressed first CARs per immune cell is 1 or less. Accordingly, with the first screening method of the present invention, it is possible to suppress selection of non-specific first CARs incapable of binding to the target antigen, for example. The number of expressed first CARs per cell can be adjusted by changing the ratio between the number of cells and the amount of the nucleic acid or vector such as a viral vector to be introduced into the cells, for example. Specifically, in the first expression step, reducing the ratio of the amount of the vector to be introduced into the certain number of cells makes it possible to reduce the number of expressed first CARs per cell. On the other hand, in the first expression step, increasing the ratio of the amount of the vector to be introduced into the certain number of cells makes it possible to increase the number of expressed first CARs per cell. In the first expression step, the efficiency of introduction of the vector into the immune cells is not particularly limited, and can be set to 10 to 60%, 10 to 50%, 20 to 40%, or 25 to 35%, for example.

Next, in the first contact step, the immune cells (candidate immune cells) obtained in the first expression step are brought into contact with the target antigen. The candidate immune cells can be brought into contact with the target antigen by culturing the candidate immune cells together with the target antigen, for example. The candidate immune cells are cultured for 6 hours to 30 days, 6 to 96 hours, or 1 to 30 days, for example. The candidate immune cells are cultured at a temperature of 28 to 37° C., for example. Hereinafter, when the immune cells are T cells, the candidate immune cells can also be referred to as “candidate CAR-Ts”, for example.

The candidate immune cells may express a single type of CAR or a plurality of types of CARs, for example. The former is preferable. It is preferable that the candidate immune cells are provided as a mixture of candidate immune cells that express CARs having different amino acid sequences, namely two or more types of candidate immune cells that express different CARs.

Examples of the target antigen to be brought into contact with the candidate immune cells include a target antigen monomer, a target antigen complex, and a target antigen-expressing cell, and a target antigen-expressing cell is preferable. An example of the target antigen complex is a target antigen multimer, and specific examples thereof include a target antigen dimer and a target antigen tetramer. The target antigen multimer can be prepared using a method in which tagged target antigens are cross-linked via an antibody, a method in which a complex of biotinylated target antigens is formed using avidin, or the like, for example. Examples of the target antigen-expressing cell include cells that intrinsically express the target antigen, and cells that express the target antigen due to the introduction of a nucleic acid coding for the target antigen. Examples of the target antigen-expressing cell include cultured cells such as 293 cells, 293 T cells, and K562 cells.

In the first contact step, the immune cells may be brought into contact with the target antigen together with a molecule for activating the immune cells, namely a costimulatory molecule. Examples of the costimulatory molecule include CD27, CD40, CD40L, CD80, CD83, CD86, OX40L, 4-1BBL, GITRL, and ICOS. Examples of the costimulatory molecule include a costimulatory molecule monomer, a costimulatory molecule complex, and a costimulatory molecule-expressing cell, and a costimulatory molecule-expressing cell is preferable. The costimulatory molecule complex can be prepared as in the case of the target antigen complex, for example.

When the target antigen-expressing cell is used as the target antigen, it is preferable that the target antigen-expressing cell also expresses a molecule for activating the immune cells, namely a costimulatory molecule. Examples of the costimulatory molecule include CD27, CD40, CD40L, CD80, CD83, CD86, OX40L, 4-1BBL, GITRL, and ICOS. The target antigen-expressing cell may express a single type of costimulatory molecule, or two or more types of costimulatory molecules. It is preferable that the target antigen-expressing cell expresses at least one selected from the group consisting of CD80, CD83, CD40, and 4-1BBL because the functions similar to those of antigen presenting cells in a living body can be exhibited and CARs that are functional in a living organism can be selected, for example.

In the first screening method of the present invention, the candidate immune cells are brought into contact with the target antigen in the first contact step. Accordingly, each of the candidate immune cells that express a CAR specific to the target antigen can continuously or intermittently form an immune synapse together with the target antigen in the same manner as in a living organism in accordance with the degree of avidity (binding ability) of the CAR for the target antigen, for example. Accordingly, the candidate immune cells that express a CAR specific to the target antigen can receive a survival signal, an activation signal, and/or a proliferation signal from the CAR in accordance with the degree of avidity (binding ability) of the CAR for the target antigen, for example. As a result, out of the candidate immune cells that have been subjected to the first contact step, candidate immune cells that express a CAR specific to the target antigen show a phenotype in which a survival or activation marker is expressed and/or the number of cells increases in accordance with the degree of avidity (binding ability) of the CAR for the target antigen, for example. Then, in the first selection step, which will be described later, the candidate immune cells showing such a phenotype are evaluated and selected, for example. Accordingly, in the first selection step, CARs can be screened based on the avidity of the CARs expressed in the candidate immune cells as well as the phenotype (functionality) shown when the CARs are activated, for example. Therefore, due to the first screening method of the present invention including the first contact step, scFvs capable of being used for CARs that are effectively functional in a living organism or immune cells can be favorably screened, for example.

The first contact step may be performed a plurality of times. The term “plurality of times” refers to 1 to 5 times or 2 or 3 times, for example, and preferably 2 or 3 times. When the first contact step is performed a plurality of times, the candidate immune cells are collected after the first contact step is performed for the first time, and then the first contact step is performed for the second time similarly to the case where the first contact step was performed for the first time. Then, the collection process and the contact process are repeated a desired number of times in the same manner. Since performing the first contact step a plurality of times makes it possible to proliferate and/or enrich candidate immune cells that express CARs capable of binding to the target antigen in the first contact step, for example, scFvs capable of binding to the target antigen can be more effectively screened. As shown in Examples, which will be described later, the CARs expressed in the candidate immune cells proliferated by performing the first contact step a plurality of times can favorably proliferate the CAR-expressing cells in a manner dependent on stimulation by the target antigen and induce the activation of target antigen-dependent cytotoxic activity and the like in the CAR-expressing cells even in the case (e.g., cancer) where the ratio of the target antigen-expressing cells to the CAR-expressing cells is high, for example. Accordingly, with the first screening method of the present invention, performing the first contact step a plurality of times makes it possible to favorably screen scFvs capable of being used for CARs that are functional more favorably in a living organism, for example.

It can also be said that, in the first contact step, out of the candidate immune cells, candidate immune cells that express CARs that recognize the target antigen are stimulated by the target antigen. Accordingly, the first contact step can also be referred to as an “antigen stimulation step”, for example.

In the first contact step, the number of the candidate immune cells is not particularly limited, and is 1×10⁵ to 1×10⁸, 1×10⁵ to 1×10⁷, 1×10⁵ to 1×10⁶, 1×10⁶ to 5×10⁶, or 1×10⁶ to 3×10⁶, for example.

When a target antigen-expressing cell is used as the target antigen in the first contact step, the cell ratio (E:T) between the candidate immune cells (E) and the target antigen-expressing cells (T) in the first contact step is 2:1 to 50:1, 5:1 to 40:1, 10:1 to 30:1, or about 20:1, for example.

Then, in the first selection step, the first scFvs of the first CARs expressed in T cells that have bound to the target antigen in the first contact step are selected as first candidate scFvs capable of binding to the target antigen. The binding of the candidate immune cells to the target antigen can be evaluated directly or indirectly, for example. The binding of the candidate immune cells to the target antigen may be indirectly evaluated because scFvs capable of inducing the activation of T cells and exhibition of functionality of T cells when used as scFvs for chimeric antigen receptors can be obtained, for example.

The direct evaluation method can be performed using a technique for detecting the binding of an antibody to an antigen such as surface plasmon resonance (SPR) or flow cytometry, for example. In a specific example, the direct evaluation method can be performed using a labeled target antigen monomer or multimer, for example. In this case, in the first selection step, the candidate immune cells are brought into contact with the labeled target antigen, for example. When the candidate immune cells express CARs capable of binding to the target antigen, the labeled target antigen and the candidate immune cell form a complex. Accordingly, in the first selection step, the candidate immune cells forming the complex that includes the label can be determined to be T cells binding to the target antigen, for example.

In a specific example of the direct evaluation method, a fluorescence-labeled target antigen multimer is mixed with the candidate immune cells to form a complex of the candidate immune cells that express a CAR specific to the target antigen and the fluorescence-labeled target antigen. Next, the thus obtained mixture is analyzed by flow cytometry, and thus the candidate immune cells that form a complex with the fluorescence-labeled target antigen are detected, evaluated, and selected based on the signal from the fluorescent label.

The indirect evaluation method can be implemented as follows, for example. T cells that express CARs capable of binding to the target antigen bind to the target antigen and are thus activated, for example. Accordingly, in the indirect evaluation method, the activation of the candidate immune cells is used as an evaluation index, for example. In a specific example, when the candidate immune cells bind to the target antigen, the expression of activation markers is increased in the immune cells, the production amounts of cytokines and/or chemokines are increased in the immune cells, and the immune cells are proliferated, for example, compared with candidate immune cells that do not bind to the target antigen. In a specific example, when the immune cells are T cells, NK cells, NKT cells, or B cells, the expression of the following activation markers is increased in the cells, the production amounts of the following cytokines and/or chemokines are increased in the cells, and the cells are proliferated, for example. Accordingly, in the first selection step, when it is determined based on any one or more of the indices that the candidate immune cells are activated, the activated immune cells can be determined to be immune cells that bind to the target antigen. The increase in the production amounts of cytokines and/or chemokines may be evaluated using a reporter whose mRNA or protein expression level increases when the expression of the cytokines and/or chemokines is induced, for example. An example of the reporter is a fluorescent protein. The above-mentioned proliferation can be evaluated by using, as an index, the attenuation of fluorescence intensity of cells that have been stained using a cell staining fluorescent dye or the like such as carboxyfluorescein succinimidyl ester (CFSE), for example.

### T Cells

Activation marker: CD69, CD107a, etc.
Cytokine, chemokine: IFN-γ, IL-12, IL-2, TNFα, MIP-1β, etc.

### NK Cells

Activation marker: CD69, CD107a, etc.
Cytokine, chemokine: INF-γ, IL-12, etc.

### NKT Cells

Activation marker: CD69, CD107a, CD25, etc.
Cytokine, chemokine: INF-γ, IL-2, etc.

### B Cells

Activation marker: CD28, CD69, CD80, CD138, B220, etc.
Cytokine, chemokine: CXCR4, etc.

Then, the first scFvs of the first CARS expressed in the candidate immune cells that have bound to the target antigen are selected as first candidate scFvs capable of binding to the target antigen. The first candidate scFvs can be selected by selecting candidate immune cells that have bound to the target antigen and reading the base sequences coding for the scFvs or CARS in the selected candidate immune cells, for example. In the first selection step, the CDRH1, the CDRH2, and the CDRH3 in the heavy-chain variable region and the CDRL1, the CDRL2, and the CDRL3 in the light-chain variable region in each of the first candidate scFvs may also be identified, for example. The CDRs can be identified using a known method referring to the genome information (e.g., the website of IMGT (http://www.imgt.org/)), for example.

Accordingly, new scFvs capable of binding to the target antigen can be screened. If a first CAR of the first CAR library meets Condition 1 above, the first screening method of the present invention can be used to screen new light-chain variable regions capable of binding to the target antigen. If a first CAR of the first CAR library meets Condition 2 above, the first screening method of the present invention can be used to screen new heavy-chain variable regions capable of binding to the target antigen.

The first screening method of the present invention may be configured such that new heavy-chain variable regions or light-chain variable regions of the first candidate scFvs are considered as the heavy-chain variable regions or light-chain variable regions of antibody and the like capable of binding to the target antigen in the first CAR library, and then the other regions are screened, for example. In this case, the first screening method of the present invention further includes a preparation step of preparing a second CAR library based on the first candidate scFvs, for example.

The second CAR library includes nucleic acids coding for second CARs, for example. Each of the second CARs includes a second antigen-binding domain, a second transmembrane domain, and a second intracellular signaling domain, for example. The second antigen-binding domain includes a second scFv to be screened for the ability to bind to the target antigen, for example.

The nucleic acids coding for the second CARs are nucleic acids (polynucleotides) coding for the amino acid sequences of the second CARs, for example.

The target antigen of the second scFvs is the same as the target antigen of the first scFvs. The second scFvs have structures similar to those of the first scFvs, for example.

Each of the second scFvs includes a second heavy-chain variable region and a second light-chain variable region, for example. The second heavy-chain variable region includes a CDRH1, a CDRH2, and a CDRH3, for example. The second light-chain variable region includes a CDRL1, a CDRL2, and a CDRL3, for example. The second heavy-chain variable region and the second light-chain variable region meet Condition 3 or Condition 4 below, for example.

### Condition 3:

If the CAR library in the first expression step meets Condition 1 above,
the CDRH1, the CDRH2, and the CDRH3 in the second heavy-chain variable region include the CDRH1, the CDRH2, and the CDRH3 in the heavy-chain variable region of a second B cell receptor, respectively, and
the CDRL1, the CDRL2, and the CDRL3 in the second light-chain variable region include the CDRL1, the CDRL2, and the CDRL3 in the light-chain variable region of the first candidate scFv, respectively.

### Condition 4:

If the CAR library in the first expression step meets Condition 2 above,
the CDRH1, the CDRH2, and the CDRH3 in the second heavy-chain variable region include the CDRH1, the CDRH2, and the CDRH3 in the heavy-chain variable region of the first candidate scFv, respectively, and
the CDRL1, the CDRL2, and the CDRL3 in the second light-chain variable region include the CDRL1, the CDRL2, and the CDRL3 in the light-chain variable region of a second B cell receptor, respectively.

Condition 3 above is employed in the case where the second CAR library is prepared using the first candidate scFvs screened using the first CAR library that meets Condition 1 above, for example. In Condition 3 above, the CDRL1, the CDRL2, and the CDRL3 of the first candidate scFv are used as those in the second light-chain variable region, and the CDRH1, the CDRH2, and the CDRH3 in the second heavy-chain variable region are screened for the ability to bind to the target antigen, for example.

If the “first B cell receptor” is changed to the “second B cell receptor”, and the “first heavy-chain variable region” is changed to the “second heavy-chain variable region”, the descriptions of the first heavy-chain variable region in Condition 2 above can be applied to the second B cell receptor in the second heavy-chain variable region, for example.

The CDRH1, the CDRH2, and the CDRH3 in the second heavy-chain variable region may respectively be polypeptides that consist of the amino acid sequences of the CDRH1, the CDRH2, and the CDRH3 of the second B cell receptor, or polypeptides that include the amino acid sequences of the CDRH1, the CDRH2, and the CDRH3 of the second B cell receptor, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the second B cell receptor, or polypeptides that include the amino acid sequences of the FRH1, the FRH2, the FRH3, and the FRH4 of the second B cell receptor, for example. It is preferable that “the second B cell receptor” in the descriptions of the CDRHs and “the second B cell receptor” in the descriptions of the FRHs are the same B cell receptor.

The second heavy-chain variable region may include the heavy-chain variable region of the second B cell receptor, for example. In this case, the second heavy-chain variable region may be a polypeptide that consists of the amino acid sequence of the heavy-chain variable region of the second B cell receptor, or a polypeptide that includes the amino acid sequence of the heavy-chain variable region of the second B cell receptor, for example.

The CDRL1, the CDRL2, and the CDRL3 in the second light-chain variable region may respectively be polypeptides that consist of the amino acid sequences of the CDRL1, the CDRL2, and the CDRL3 of the first candidate scFv, or polypeptides that include the amino acid sequences of the CDRL1, the CDRL2, and the CDRL3 of the first candidate scFv, for example.

The FRs in the second light-chain variable region may include the FRs in the light-chain variable region of the first candidate scFv, for example. The FRL1, the FRL2, the FRL3, and the FRL4 in the second light-chain variable region may respectively be polypeptides that consist of the amino acid sequences of the FRL1, the FRL2, the FRL3, and the FRL4 of the first candidate scFv, or polypeptides that include the amino acid sequences of the FRL1, the FRL2, the FRL3, and the FRL4 of the first candidate scFv, for example. It is preferable that “the first candidate scFv” in the descriptions of the CDRLs and “the first candidate scFv” in the descriptions of the FRLs are the same scFv.

The second light-chain variable region may include the light-chain variable region of the first candidate scFv, for example. In this case, the second light-chain variable region may be a polypeptide that consists of the amino acid sequence of the light-chain variable region of the first candidate scFv, or a polypeptide that includes the amino acid sequence of the light-chain variable region of the first candidate scFv, for example.

Next, Condition 4 above is employed in the case where the second CAR library is prepared using the first candidate scFvs screened using the first CAR library that meets Condition 2 above, for example. In Condition 4 above, the CDRH1, the CDRH2, and the CDRH3 of the first candidate scFv are used as those in the second heavy-chain variable region, and the CDRL1, the CDRL2, and the CDRL3 in the second light-chain variable region are screened for the ability to bind to the target antigen, for example.

The CDRH1, the CDRH2, and the CDRH3 in the second heavy-chain variable region may respectively be polypeptides that consist of the amino acid sequences of the CDRH1, the CDRH2, and the CDRH3 of the first candidate scFv, or polypeptides that include the amino acid sequences of the CDRH1, the CDRH2, and the CDRH3 of the first candidate scFv, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the first candidate scFv, or polypeptides that include the amino acid sequences of the FRH1, the FRH2, the FRH3, and the FRH4 of the first candidate scFv, for example. It is preferable that “the first candidate scFv” in the descriptions of the CDRHs and “the first candidate scFv” in the descriptions of the FRHs are the same scFv.

The second heavy-chain variable region may include the heavy-chain variable region of the first candidate scFv, for example. In this case, the second heavy-chain variable region may be a polypeptide that consists of the amino acid sequence of the heavy-chain variable region of the first candidate scFv, or a polypeptide that includes the amino acid sequence of the heavy-chain variable region of the first candidate scFv, for example.

If the “first B cell receptor” is changed to the “second B cell receptor”, and the “first light-chain variable region” is changed to the “second light-chain variable region”, the descriptions of the first light-chain variable region in Condition 1 above can be applied to the second B cell receptor in the second light-chain variable region, for example.

The CDRL1, the CDRL2, and the CDRL3 in the second light-chain variable region may respectively be polypeptides that consist of the amino acid sequences of the CDRL1, the CDRL2, and the CDRL3 of the second B cell receptor, or polypeptides that include the amino acid sequences of the CDRL1, the CDRL2, and the CDRL3 of the second B cell receptor, for example.

The FRL1, the FRL2, the FRL3, and the FRL4 in the second light-chain variable region may respectively be polypeptides that consist of the amino acid sequences of the FRL1, the FRL2, the FRL3, and the FRL4 of the second B cell receptor, or polypeptides that include the amino acid sequences of the FRL1, the FRL2, the FRL3, and the FRL4 of the second B cell receptor, for example. It is preferable that “the second B cell receptor” in the descriptions of the CDRLs and “the second B cell receptor” in the descriptions of the FRLs are the same B cell receptor.

The second light-chain variable region may include the light-chain variable region of the second B cell receptor, for example. In this case, the second light-chain variable region may be a polypeptide that consists of the amino acid sequence of the light-chain variable region of the second B cell receptor, or a polypeptide that includes the amino acid sequence of the light-chain variable region of the second B cell receptor, for example.

In the second scFv, the second heavy-chain variable region and the second light-chain variable region are coupled to each other via the above-described linker peptide (Fv linker peptide), for example. The Fv linker peptide in the second scFv may be the same as or different from the Fv linker peptide in the first scFv, for example.

The second antigen-binding domain includes the second scFv, and the second binding domain may also include a structure other than the scFv that includes the heavy-chain variable region and the light-chain variable region. In a specific example, the second binding domain may be a Fab, Fab′, F(ab′)₂, variable region fragment (Fv), disulfide-bond Fv, or the like.

In the second CAR, the second transmembrane domain and the second intracellular signaling domain may be the same as or different from the first transmembrane domain and the first intracellular signaling domain in the first CAR library, respectively. The descriptions of the first transmembrane domain and the first intracellular signaling domain in the CAR library of the present invention can be applied to the second transmembrane domain and the second intracellular signaling domain in the second CAR library, respectively, for example.

In the second CAR, the domains may be coupled to each other via a linker peptide (domain linker peptide), for example. The domain linker peptide is constituted by 1 to 40, 1 to 18, 1 to 15, 1 to 7, 1 to 3, or 1 or 2 amino acids, for example. The domain linker peptide is constituted by amino acids such as glycine and serine, for example, and a specific example thereof is (GGGGS)ₙ. n in this formula is an integer from 1 to 6, for example.

An example of the second CAR is a polypeptide that consists of the amino acid sequence (Sequence ID No. 15) represented by Formula (1) above. In Formula (1) above, V₁ and V₂ are the amino acid sequences of the heavy-chain variable region and the light-chain variable region, respectively, or the amino acid sequences of the light-chain variable region and the heavy-chain variable region, respectively. It is preferable that the order of the heavy-chain variable region and the light-chain variable region in the second CAR is opposite to the order of the heavy-chain variable region and the light-chain variable region in the first CAR because the second CAR library can be prepared more easily, for example. In a specific example, if the heavy-chain variable region and the light-chain variable region are arranged in this order from the N terminus in the first CAR, the light-chain variable region and the heavy-chain variable region are arranged in this order from the N terminus in the second CAR, for example. If the light-chain variable region and the heavy-chain variable region are arranged in this order from the N terminus in the first CAR, the heavy-chain variable region and the light-chain variable region are arranged in this order from the N terminus in the second CAR, for example.

An example of the nucleic acid coding for the second CAR is a polynucleotide consisting of the base sequence (Sequence ID No. 16) represented by Formula (2) above. In Formula (2) above, N₁ and N₂ are the base sequences coding for the heavy-chain variable region and the light-chain variable region, respectively, or the base sequences coding for the light-chain variable region and the heavy-chain variable region, respectively.

It is preferable that the second CAR library includes a plurality of types of nucleic acids, for example. In this case, the second CAR library is a mixture of a plurality of types of nucleic acids, for example. It is preferable that some or all of the plurality of types of nucleic acids code for different second CARs, and preferably code for different second antigen-binding domains, for example. When the plurality of types of nucleic acids code for different second antigen-binding domains, the regions other than the second antigen-binding domains in the second CARs have the same amino acid sequence or different amino acid sequences, for example. The number of types of nucleic acids included in the second CAR library is 1×10⁵ to 1×10⁷, 1×10⁵ to 1×10⁶, or 1×10⁶ to 5×10⁶, for example, and preferably about 2×10⁶ (e.g., 1×10⁶ to 3×10⁶).

The second CAR may include a signal peptide at the N terminus, for example. The second CAR may include a tag, for example. If the “first CAR” is changed to the “second CAR”, the descriptions of the signal peptide and the tag in the first CAR can be applied to the above-mentioned signal peptide and the tag, for example.

In the present invention, the nucleic acids coding for the second CARs can be prepared based on the amino acid sequences of the second CARs using an ordinary method, for example. In a specific example, the nucleic acids coding for the second CARs can be prepared based on the base sequences coding for the amino acid sequences obtained from the database in which the amino acid sequences of the above-described domains are registered, using a molecular biological technique and/or a chemical synthesis method, for example. The base sequences of the nucleic acids may be subjected to codon optimization in accordance with the source of cells in which the second CAR library of the present invention is to be expressed, for example.

The nucleic acids coding for the second CARs may be introduced into expression vectors, for example. The descriptions of the expression vectors in the CAR library of the present invention can be applied to the above-mentioned expression vectors, for example.

Next, in the first screening method of the present invention, an expression step, a contact step, and a selection step are performed in the same manner, except that the second CAR library prepared in the preparation step is used instead of the first CAR library, for example. Specifically, the first screening method of the present invention further includes: a second expression step of expressing the second CAR library in immune cells; a second contact step of bringing the immune cells obtained in the second expression step into contact with the target antigen; and a second selection step of selecting the second scFvs of the CARs expressed in the immune cells that have bound to the target antigen in the second contact step as second candidate scFvs capable of binding to the target antigen, for example. The immune cells in the first expression step, the first contact step, and the first selection step and the immune cells in the second expression step, the second contact step, and the second selection step may be the same or different. The immune cells are preferably T cells or T cell-like cells.

If the “first expression step” is changed to the “second expression step”, the “first CAR library” is changed to the “second CAR library”, and the “first CAR” is changed to the “second CAR”, the descriptions of the first expression step can be applied to the second expression step, for example.

If the “first expression step” is changed to the “second expression step”, and the “first contact step” is changed to the “second contact step”, the descriptions of the first contact step can be applied to the second contact step, for example.

If the “first contact step” is changed to the “second contact step”, the “first selection step” is changed to the “second selection step”, the “first scFv” is changed to the “second scFv”, the “first candidate scFv” is changed to the “second candidate scFv”, and the “first CAR” is changed to the “second CAR”, the descriptions of the first selection step can be applied to the second selection step, for example.

Thus, with the first screening method of the present invention, scFvs that include new heavy-chain variable regions and light-chain variable regions capable of binding to the target antigen can be screened, for example.

The first screening method of the present invention may include a designing step of designing antibodies or antigen-binding fragments thereof based on the first candidate scFvs or the second candidate scFvs. The designing step can be performed by grafting the CDRH1, the CDRH2, and the CDRH3 into the heavy-chain variable region and the CDRL1, the CDRL2, and the CDRL3 in the light-chain variable region of the first candidate scFv or the second candidate scFv into a new antibody or an antigen-binding fragment thereof, for example. Specifically, the designing step can be performed by respectively using the amino acid sequences of the CDRH1, the CDRH2, and the CDRH3 of the first candidate scFv or the second candidate scFv and the amino acid sequences of the CDRL1, the CDRL2, and the CDRL3 in the light-chain variable region of the first candidate scFv or the second candidate scFv as the amino acid sequences of the CDRH1, the CDRH2, and the CDRH3 and the amino acid sequences of the CDRL1, the CDRL2, and the CDRL3 in the light-chain variable region of a new antibody or an antigen-binding fragment thereof, for example. The CDR grafting in the designing step may be performed on a single CDR or a plurality of CDRs of the first candidate scFv or the second candidate scFv, for example, and is preferably performed on all of the CDRs. The descriptions of the types of antibodies of the present invention or antigen-binding fragments thereof, which will be described later, can be applied to the type of the above-mentioned new antibody and the antigen-binding fragment thereof, for example.

In the designing step, the FRH1, the FRH2, the FRH3, and the FRH4 in the heavy-chain variable region and the FRL1, the FRL2, the FRL3, and the FRL4 in the light-chain variable region of the first candidate scFv or the second candidate scFv may be grafted into a new antibody or an antigen-binding fragment thereof. In this case, in the designing step, the amino acid sequences of the FRH1, the FRH2, the FRH3, and the FRH4 of the first candidate scFv or the second candidate scFv and the amino acid sequences of the FRL1, the FRL2, the FRL3, and the FRL4 in the light-chain variable region of the first candidate scFv or the second candidate scFv can be respectively used as the amino acid sequences of the FRH1, the FRH2, the FRH3, and the FRH4 and the amino acid sequences of the FRL1, the FRL2, the FRL3, and the FRL4 in the light-chain variable region of the new antibody or the antigen-binding fragment thereof, for example. The FR grafting in the designing step may be performed on a single FR or a plurality of FRs of the first candidate scFv or the second candidate scFv, for example, and is preferably performed on all of the FRs.

The first screening method of the present invention includes the first expression step, but is not limited thereto in the present invention. The first screening method may have a configuration in which the first expression step is not included. In this case, the first screening method of the present invention can be started from the first contact step by using immune cells (CAR library cells) that are prepared in advance and express the CAR library of the present invention, for example.

### CAR Library Cells

The chimeric antigen receptor (CAR) library cells of the present invention include cells that express the CAR library of the present invention. The CAR library cells of the present invention are characterized by including cells that express the CAR library of the present invention, and there is no particular limitation on the other configurations and conditions. With the CAR library cell of the present invention, the first screening method of the present invention can be favorably implemented. The descriptions of the CAR library and the first screening method of the present invention can be applied to the CAR library cells of the present invention.

The number of the CAR library cells of the present invention is 1×10⁵ to 1×10⁸, 1×10⁵ to 1×10⁷, 1×10⁵ to 1×10⁶, 1×10⁶ to 5×10⁶, or 1×10⁶ to 3×10⁶, for example. It is preferable that CARs expressed in the CAR library cells are different CARs that are included in the CAR library of the present invention, for example. The CAR library cells may include cells that do not express the CAR library of the present invention, namely cells into which the CAR library is not introduced, for example.

When the number of the CAR library cells of the present invention used is 1×10⁶, the number of types of expressed CARs is 1×10⁴ to 1×10⁷, 1×10⁵ to 1×10⁶, 1×10⁶ to 5×10⁶, or 1×10⁵ to 1×10⁶, for example.

The CAR library cells of the present invention can be manufactured in the same manner as in the first expression step of the first screening method of the present invention, for example. The CAR library cells of the present invention are preferably cells derived from immune cells, for example, and are preferably cells derived from T cells, NK cells, or NKT cells.

### First Antibody and Antigen-Binding Fragment Thereof

As described above, the antibody of the present invention against the complex of HLA-A*02:01 and NY-ESO-1₁₅₇₋₁₆₅ (also referred to as “A2/NY-ESO-1₁₅₇” hereinafter) or the antigen-binding fragment thereof includes the heavy-chain variable region of (H) below and the light-chain variable region of (L) below. The antibody of the present invention or the like is characterized by including the heavy-chain variable region of (H) below and the light-chain variable region of (L) below, and there is no particular limitation on the other configurations and conditions. The antibody of the present invention or the like is capable of binding to A2/NY-ESO-1₁₅₇. It is known that A2/NY-ESO-1₁₅₇ is expressed in specific cancer cells derived from lung cancer, malignant melanoma, synovial sarcoma, myeloma, and the like, for example. Accordingly, the antibody of the present invention or the like can be favorably used as a bispecific antibody against A2/NY-ESO-1₁₅₇-expressing cancer cells, the antigen-binding domain of a CAR in CAR-T cells, and the like, for example. The descriptions of the CAR library, the first screening method, and the like of the present invention can be applied.

(H) A heavy-chain variable region that includes
a heavy-chain complementarity determining region (CDRH) 1, a CDRH2, and a CDRH3,
wherein the CDRH1 is a polypeptide that includes an amino acid sequence of (H1),
the CDRH2 is a polypeptide that includes an amino acid sequence of (H2),
the CDRH3 is a polypeptide that includes an amino acid sequence of (H3), and
the amino acid sequences of (H1), (H2), and (H3) are as follows:
(H1) an amino acid sequence of (H1-1), (H1-2), or (H1-3) below:
   (H1-1) any one of amino acid sequences of CDRH1 shown in Table 1A below,
   (H1-2) an amino acid sequence having 80% or more identity to the amino acid sequence of (H1-1), and
   (H1-3) an amino acid sequence consisting of the amino acid sequence of (H1-1) with deletion, substitution, insertion, and/or addition of one or several amino acids,
(H2) an amino acid sequence of (H2-1), (H2-2), or (H2-3) below:
   (H2-1) any one of amino acid sequences of CDRH2 shown in Table 1A below,
   (H2-2) an amino acid sequence having 80% or more identity to the amino acid sequence of (H2-1), and
   (H2-3) an amino acid sequence consisting of the amino acid sequence of (H2-1) with deletion, substitution, insertion, and/or addition of one or several amino acids; and
(H3) an amino acid sequence of (H3-1), (H3-2), or (H3-3) below:
   (H3-1) any one of amino acid sequences of CDRH3 shown in Table 1A below,
   (H3-2) an amino acid sequence having 80% or more identity to the amino acid sequence of (H3-1), and
   (H3-3) an amino acid sequence consisting of the amino acid sequence of (H3-1) with deletion, substitution, insertion, and/or addition of one or several amino acids.

(L) A light-chain variable region that includes
a light-chain complementarity determining region (CDRL) 1, a CDRL2, and a CDRL3,
wherein the CDRL1 is a polypeptide that includes an amino acid sequence of (L1),
the CDRL2 is a polypeptide that includes an amino acid sequence of (L2),
the CDRL3 is a polypeptide that includes an amino acid sequence of (L3), and
the amino acid sequences of (L1), (L2), and (L3) are as follows:
(L1) an amino acid sequence of (L1-1), (L1-2), or (L1-3) below:
   (L1-1) any one of amino acid sequences of CDRL1 shown in Table 1B below,
   (L1-2) an amino acid sequence having 80% or more identity to the amino acid sequence of (L1-1), and
   (L1-3) an amino acid sequence consisting of the amino acid sequence of (L1-1) with deletion, substitution, insertion, and/or addition of one or several amino acids;
(L2) an amino acid sequence of (L2-1), (L2-2), or (L2-3) below:
   (L2-1) any one of amino acid sequences of CDRL2 shown in Table 1B below,
   (L2-2) an amino acid sequence having 80% or more identity to the amino acid sequence of (L2-1), and
   (L2-3) an amino acid sequence consisting of the amino acid sequence of (L2-1) with deletion, substitution, insertion, and/or addition of one or several amino acids; and
(L3) an amino acid sequence of (L3-1), (L3-2), or (L3-3) below:
   (L3-1) any one of amino acid sequences of CDRL3 shown in Table 1B below,
   (L3-2) an amino acid sequence having 80% or more identity to the amino acid sequence of (L3-1), and
   (L3-3) an amino acid sequence consisting of the amino acid sequence of (L3-1) with deletion, substitution, insertion, and/or addition of one or several amino acids.

TABLE 1A

| Heavy- | | | | |
|---|---|---|---|---|
| chain | | | | |
| variable | | | | |
| region | CDRH1 | CDRH2 | CDRH3 | |
| (HA) | GGSIS | VSYSG | ARESY | H1 |
| | SNY | ST | YYYGM | |
| | (Sequence | (Sequence | DV | |
| | ID | ID | (Sequence | |
| | No. | No. | ID | |
| | 17) | 18) | No. | |
| | | | 19) | |
| (HB) | GFTFS | IVSSG | AGELL | 3M4E5H |
| | TYQ | GST | PYYGM | |
| | (Sequence | (Sequence | DV | |
| | ID | ID | (Sequence | |
| | No. | No. | ID | |
| | 21) | 22) | No. | |
| | | | 23) | |
| (HC) | GGSIS | INHSG | ARCPT | H73 |
| | SYY | ST | YYYGM | |
| | (Sequence | (Sequence | DV | |
| | ID | ID | (Sequence | |
| | No. | No. | ID | |
| | 25) | 26) | No. | |
| | | | 27) | |

TABLE 1B

| Light-chain | | | | |
|---|---|---|---|---|
| variable region | CDRL1 | CDRL2 | CDRL3 | |
| (LA) | SRDVGGYNY | DVI | WSFAGSYYV | 3M4E5L |
| | (Sequence ID No. 29) | (Sequence ID No. 30) | (Sequence ID No. 31) | |
| (LB) | QSISSY | AAS | QQYYSTPQT | K52 |
| | (Sequence ID No. 33) | (Sequence ID No. 34) | (Sequence ID No. 35) | |
| (LC) | QSISSY | AAS | QQYESYRRS | K73 |
| | (Sequence ID No. 37) | (Sequence ID No. 38) | (Sequence ID No. 39) | |
| (LD) | QSISSY | AAS | QQYNSYSRT | K124 |
| | (Sequence ID No. 41) | (Sequence ID No. 42) | (Sequence ID No. 43) | |
| (LE) | QSISSY | AAS | QQYNSYSPCT | K125 |
| | (Sequence ID No. 46) | (Sequence ID No. 47) | (Sequence ID No. 48) | |
| (LF) | QDISRY | AAS | QQYDNLIT | K131 |
| | (Sequence ID No. 50) | (Sequence ID No. 51) | (Sequence ID No. 52) | |
| (LG) | QDISRY | AAS | QQYNSYSRT | K145 |
| | (Sequence ID No. 54) | (Sequence ID No. 55) | (Sequence ID No. 56) | |
| (LH) | QSISSY | AAS | QQYDNLIT | K151 |
| | (Sequence ID No. 58) | (Sequence ID No. 59) | (Sequence ID No. 60) | |
| (LI) | QSVSSN | GAS | QQYNSYSRT | K160 |
| | (Sequence ID No. 62) | (Sequence ID No. 63) | (Sequence ID No. 64) | |
| (LJ) | QSISSY | AAS | QQYESYSRT | K173 |
| | (Sequence ID No. 66) | (Sequence ID No. 67) | (Sequence ID No. 68) | |
| (LK) | SSDVGGYDF | DVN | SSYAGSNSV | L1 |
| | (Sequence ID No. 70) | (Sequence ID No. 71) | (Sequence ID No. 72) | |
| (LL) | SSDVGGYEF | DVI | SSYTSSSTYV | L66 |
| | (Sequence ID No. 74) | (Sequence ID No. 75) | (Sequence ID No. 76) | |
| (LM) | GSDVGAYDY | DVS | SSYSGSSTWV | L73 |
| | (Sequence ID No. 78) | (Sequence ID No. 79) | (Sequence ID No. 80) | |
| (LN) | SSDVGSYNL | DVS | SSYTSSSTFAV | L80 |
| | (Sequence ID No. 82) | (Sequence ID No. 83) | (Sequence ID No. 84) | |
| (LO) | SSDVGGYNY | DVS | CSYAGGYYV | L88 |
| | (Sequence ID No. 86) | (Sequence ID No. 87) | (Sequence ID No. 88) | |
| (LP) | SSDVGGYNY | DVS | SSYAGSGSTPFV | L102 |
| | (Sequence ID No. 90) | (Sequence ID No. 91) | (Sequence ID No. 92) | |
| (LQ) | SSDVGGYNY | DVS | CSYAGRRYV | L124 |
| | (Sequence ID No. 94) | (Sequence ID No. 95) | (Sequence ID No. 96) | |

In the present invention, the term “HLA-A*02:01” means a class-I antigen derived from the A*02:01 allele of a human major histocompatibility complex (MHC), for example. HLA-A*02:01 forms a complex with human β2-microglobulin, for example. An example of the amino acid sequence of HLA-A*02:01 is the amino acid sequence registered as NCBI Accession No. HG794376. An example of the amino acid sequence of human B2-microglobulin is the amino acid sequence registered as NCBI Accession No. NM 004048.2.

In the present invention, the term “NY-ESO-1₁₅₇₋₁₆₅” means a peptide constituted by amino acids between position 157 and position 165 (SLLMWITQC (Sequence ID No. 206)) of the amino acid sequence of the NY-ESO-1 protein, for example. NY-ESO-1 is a cancer antigen expressed in cancers such as human lung cancer, malignant melanoma, synovial sarcoma, and myeloma, for example. An example of the amino acid sequence of the NY-ESO-1 protein is the amino acid sequence registered as NCBI Accession No. NM_001327.2.

In the present invention, the term “A2/NY-ESO-1₁₅₇” means a complex formed by HLA-A*02:01 and NY-ESO-1₁₅₇₋₁₆₅, for example, and specifically a complex in which NY-ESO-1₁₅₇-165 binds to the peptide-binding groove of HLA-A*02:01. A2/NY-ESO-1₁₅₇ is preferably a complex in which NY-ESO-1₁₅₇₋₁₆₅ binds to the peptide-binding groove of the complex of HLA-A*02:01 and human β2-microglobulin.

The antibody of the present invention may be a so-called “antibody” having an immunoglobulin molecular structure, or an antigen-binding fragment thereof, for example. The antibody of the present invention or the like need only include the heavy-chain variable region and the light-chain variable region described above. If the present invention is directed to an antibody, there is no particular limitation on the immunoglobulin class and isotype thereof, for example. Examples of the immunoglobulin class include IgG, IgM, IgA, IgD, and IgE. Examples of the IgG include IgG1, IgG2, IgG3, and IgG4.

The antibody may be a monoclonal antibody, a polyclonal antibody, a recombinant antibody, a human antibody (e.g., fully human antibody), a humanized antibody, a chimeric antibody, or a multispecific antibody, for example.

The term “antigen-binding fragment” as used in the present invention means a part (e.g., a partial fragment) of the antibody that recognizes (is capable of binding to) A2/NY-ESO-1₁₅₇ above. Examples of the antigen-binding fragment include Fabs, Fab's, F(ab′)₂s, variable region fragments (Fvs), disulfide-bond Fvs, single-chain Fvs (scFvs), bispecific antibodies, and polymers thereof. In each of the scFvs, the heavy-chain variable region and the light-chain variable region are coupled to each other via a linker, for example. The heavy-chain variable region, the linker, and the light-chain variable region may be arranged in the stated order from the N terminus, or in the inverse order from the N terminus.

The antibody of the present invention or the like may include a constant region in addition to the heavy-chain variable region and the light-chain variable region described above, and the constant region is a human constant region or a mouse constant region, for example. In the case of an antibody (immunoglobulin), the constant region of the heavy chain includes the CH1 region, the CH2 region, and the CH3 region, for example, and the constant region of the light chain includes the CL region, for example. If the antibody of the present invention or the like includes the constant region, the heavy-chain variable region binds to at least one of the CH1, the CH2, and the CH3, and the light-chain variable region binds to the CL, for example. In such a case, the heavy-chain variable region directly binds to the CH1, for example.

In general, the heavy chain and the light chain in an antibody molecule each include three complementarity determining regions (CDRs). The CDRs are also referred to as “hypervariable domains”. The CDRs are regions in which the primary structure is particularly likely to be variable in the variable regions of the heavy chain and the light chain, and the primary structure generally includes three CDRs. In the present invention, the three CDRs in the heavy chain are referred to as a heavy-chain CDR 1 (CDRH1), a heavy-chain CDR 2 (CDRH2), and a heavy-chain CDR 3 (CDRH3), in this order from the amino terminus of the amino acid sequence of the heavy chain, and the three CDRs in the light-chain are referred to as a light-chain CDR 1 (CDRL1), a light-chain CDR 2 (CDRL2), and a light-chain CDR 3 (CDRL3), in this order from the amino terminus of the amino acid sequence of the light chain. These sites are close to one another in the three-dimensional structure and determine the binding specificity for an antigen.

In the heavy-chain variable region of (H) above, the CDRH1 is the CDRH1 of (HA), (HB), or (HC) above. The CDRH2 is the CDRH2 of (HA), (HB), or (HC) above. The CDRH3 is the CDRH3 of (HA), (HB), or (HC) above.

In the light-chain variable region of (L) above, the CDRL1 is the CDRL1 of any one of (LA) to (LQ) above. The CDRL2 is the CDRL2 of any one of (LA) to (LQ) above. The CDRL3 is the CDRL3 of any one of (LA) to (LQ) above.

The term “identity” as used for the CDRs refers to the degree of identity when appropriately aligning sequences to be compared, for example, and means the ratio (%) of exactly the same amino acids in these sequences. The “identity” in each case refers to 80% or more, 85% or more, 90% or more, 95% or more, 96% or more, 97% or more, 98% or more, or 99% or more identity, for example. The identity can be calculated with default parameters using analysis software such as BLAST or FASTA (the same applies hereinafter).

The term “one or several” as used for the CDRs regarding substitution and the like refers to 1 to 5, 1 to 4, 1 to 3, 1 or 2, or 1, for example.

The amino acid substitution may be conservative substitution, for example (the same applies hereinafter). The term “conservative substitution” means that one or several amino acids are substituted by other amino acids and/or amino acid derivatives such that the functions of a protein are not substantially modified. It is preferable that a “substituting amino acid” and an “amino acid to be substituted” have similar properties and/or functions, for example. Specifically, it is preferable that they are similar in chemical properties such as a hydrophobicity/hydrophilicity index (hydropathy), a polarity, and an electric charge, physical properties such as a secondary structure, and the like, for example. Amino acids or amino acid derivatives having similar properties and/or functions are known in the art, for example. Specific examples of nonpolar amino acids (hydrophobic amino acids) include alanine, valine, isoleucine, leucine, proline, tryptophan, phenylalanine, and methionine; specific examples of polar amino acids (neutral amino acids) include glycine, serine, threonine, tyrosine, glutamine, asparagine, and cysteine; specific examples of positively charged amino acids (basic amino acids) include arginine, histidine, and lysine; and specific examples of negatively charged amino acids (acidic amino acids) include aspartic acid and glutamic acid.

In the heavy-chain variable region of (H) above, there is no particular limitation on the combination of (H1-1), (H2-1), and (H3-1) above, and the CDRH1 of (HA), (HB), or (HC) above, the CDRH2 of (HA), (HB), or (HC) above, and the CDRH3 of (HA) or (HB) above can be combined as desired, for example. The combination of (H1-1), (H2-1), and (H3-1) above is preferably a combination of the CDRH1, the CDRH2, and the CDRH3 of (HA), (HB), or (HC) above.

In the light-chain variable region of (L) above, there is no particular limitation on the combination of (L1-1), (L2-1), and (L3-1) above, and the CDRL1 of any one of (LA) to (LQ) above, the CDRL2 of any one of (LA) to (LQ) above, and the CDRL3 of any one of (LA) to (LQ) above can be combined as desired, for example. The combination of (L1-1), (L2-1), and (L3-1) above is preferably a combination of the CDRL1, the CDRL2, and the CDRL3 of any one of (LA) to (LQ) above.

There is no particular limitation on the combination of (H1-1), (H2-1), and (H3-1) above and (L1-1), (L2-1), and (L3-1) above, and the CDRH1 of (HA), (HB), or (HC) above, the CDRH2 of (HA), (HB), or (HC) above, and the CDRH3 of (HA), (HB), or (HC) above, and the CDRL1 of any one of (LA) to (LQ) above, the CDRL2 of any one of (LA) to (LQ) above, and the CDRL3 of any one of (LA) to (LQ) above can be combined as desired, for example. The combination of (H1-1), (H2-1), and (H3-1) above and (L1-1), (L2-1), and (L3-1) above is preferably a combination of the CDRH1, the CDRH2, and the CDRH3 of (HA), (HB), or (HC) above and the CDRL1, the CDRL2, and the CDRL3 of any one of (LA) to (LQ) above, and more preferably a combination shown in Table 2 below.

TABLE 2

| | Heavy-chain | Light-chain |
|---|---|---|
| Combination | variable region | variable region |
| (1) | (HA) | (LA) |
| (2) | (HA) | (LB) |
| (3) | (HA) | (LC) |
| (4) | (HA) | (LD) |
| (5) | (HA) | (LE) |
| (6) | (HA) | (LF) |
| (7) | (HA) | (LG) |
| (8) | (HA) | (LH) |
| (9) | (HA) | (LI) |
| (10) | (HA) | (LJ) |
| (11) | (HB) | (LK) |
| (12) | (HB) | (LL) |
| (13) | (HB) | (LM) |
| (14) | (HB) | (LN) |
| (15) | (HB) | (LO) |
| (16) | (HB) | (LP) |
| (17) | (HB) | (LQ) |
| (18) | (HC) | (LA) |

Hereinafter, the combination of the heavy-chain variable region or heavy chain and the light-chain variable region or light chain in the antibody of the present invention or the like will be described more specifically. In this combination, the descriptions of the heavy-chain variable region can be applied to the heavy chain, and vice versa. Also, in this combination, the descriptions of the light-chain variable region can be applied to the light chain, and vice versa. In the amino acid sequences and the base sequences shown below, underlined amino acid sequences and base sequences are amino acid sequences that correspond to the CDRs and base sequences coding for the amino acid sequences that correspond to the CDRs, respectively, unless otherwise stated.

As described above, the combination of the heavy-chain variable region and the light-chain variable region in the antibody of the present invention or the like is one of the combinations of (1) to (18) above, for example.

### Combination (1)

The antibodies or the like of Combination (1) are also referred to as an antibody H1-3M4E5L group, for example. In Combination (1) above, the heavy-chain variable region of (HA) above includes the CDRH1, the CDRH2, and the CDRH3, the CDRH1 is a polypeptide that includes the amino acid sequence of (H1-A) below, the CDRH2 is a polypeptide that includes the amino acid sequence of (H2-A) below, and the CDRH3 is a polypeptide that includes the amino acid sequence of (H3-A) below. The light-chain variable region of (LA) above includes the CDRL1, the CDRL2, and the CDRL3, the CDRL1 is a polypeptide that includes the amino acid sequence of (L1-A) below, the CDRL2 is a polypeptide that includes the amino acid sequence of (L2-A) below, and the CDRL3 is a polypeptide that includes the amino acid sequence of (L3-A) below.

(H1-A) An amino acid sequence of (H1-A1), (H1-A2), or (H1-A3) below:
(H1-A1) an amino acid sequence of Sequence ID No. 17 (GGSISSNY),
(H1-A2) an amino acid sequence having 80% or more identity to the amino acid sequence of Sequence ID No. 17, and
(H1-A3) an amino acid sequence consisting of the amino acid sequence of Sequence ID No. 17 with deletion, substitution, insertion, and/or addition of one or several amino acids.

(H2-A) An amino acid sequence of (H2-A1), (H2-A2), or (H2-A3) below:
(H2-A1) an amino acid sequence of Sequence ID No. 18 (VSYSGST),
(H2-A2) an amino acid sequence having 80% or more identity to the amino acid sequence of Sequence ID No. 18, and
(H2-A3) an amino acid sequence consisting of the amino acid sequence of Sequence ID No. 18 with deletion, substitution, insertion, and/or addition of one or several amino acids.

(H3-A) An amino acid sequence of (H3-A1), (H3-A2), or (H3-A3) below:
(H3-A1) an amino acid sequence of Sequence ID No. 19 (ARESYYYYGMDV),
(H3-A2) an amino acid sequence having 80% or more identity to the amino acid sequence of Sequence ID No. 19, and
(H3-A3) an amino acid sequence consisting of the amino acid sequence of Sequence ID No. 19 with deletion, substitution, insertion, and/or addition of one or several amino acids.

(L1-A) An amino acid sequence of (L1-A1), (L1-A2), or (L1-A3) below:
(L1-A1) an amino acid sequence of Sequence ID No. 29 (SRDVGGYNY),
(L1-A2) an amino acid sequence having 80% or more identity to the amino acid sequence of Sequence ID No. 29, and
(L1-A3) an amino acid sequence consisting of the amino acid sequence of Sequence ID No. 29 with deletion, substitution, insertion, and/or addition of one or several amino acids.

(L2-A) An amino acid sequence of (L2-A1), (L2-A2), or (L2-A3) below:
(L2-A1) an amino acid sequence of Sequence ID No. 30 (DVI),
(L2-A2) an amino acid sequence having 80% or more identity to the amino acid sequence of Sequence ID No. 30, and
(L2-A3) an amino acid sequence consisting of the amino acid sequence of Sequence ID No. 30 with deletion, substitution, insertion, and/or addition of one or several amino acids.

(L3-A) An amino acid sequence of (L3-A1), (L3-A2), or (L3-A3) below:
(L3-A1) an amino acid sequence of Sequence ID No. 31 (WSFAGSYYV),
(L3-A2) an amino acid sequence having 80% or more identity to the amino acid sequence of Sequence ID No. 31, and
(L3-A3) an amino acid sequence consisting of the amino acid sequence of Sequence ID No. 31 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (1) above, the heavy-chain variable region of (HA) above includes a polypeptide consisting of the amino acid sequence of (H-A) below, for example. The light-chain variable region of (LA) above includes a polypeptide consisting of the amino acid sequence of (L-A) below, for example.

(H-A) An amino acid sequence of (H-A1), (H-A2), or (H-A3) below:
(H-A1) an amino acid sequence of Sequence ID No. 20:

Sequence ID No. 20:
QVQLQESGPGLVKPSDTLSLTCLVSGGSIS
SNYWSWIRQAPGKGLEWIGHVSYSGSTNY
NPSLKSRVTISVDTSKNQFSLKLSSVTAAD
TAVYYCARESYYYYGMDVWGQGTTVTVSS,

(H-A2) an amino acid sequence having 80% or more identity to the amino acid sequence of Sequence ID No. 20, and
(H-A3) an amino acid sequence consisting of the amino acid sequence of Sequence ID No. 20 with deletion, substitution, insertion, and/or addition of one or several amino acids.

(L-A) An amino acid sequence of (L-A1), (L-A2), or (L-A3) below:
(L-A1) an amino acid sequence of Sequence ID No. 32:

Sequence ID No. 32:
QSELTQPRSVSGSPGQSVTISCTGTSRDVG
GYNYVSWYQQHPGKAPKLIIHDVIERSSG
VPDRFSGSKSGNTASLTISGLQAEDEADYY
CWSFAGSYYVFGTGTDVTVL,

(L-A2) an amino acid sequence having 80% or more identity to the amino acid sequence of Sequence ID No. 32, and
(L-A3) an amino acid sequence consisting of the amino acid sequence of Sequence ID No. 32 with deletion, substitution, insertion, and/or addition of one or several amino acids.

The amino acid sequence of (H-A1) above is a sequence that includes the amino acid sequences of (H1-A1) of the CDRH1, (H2-A1) of the CDRH2, and (H3-A1) of the CDRH3, for example. The amino acid sequence of (H-A2) above may be an amino acid sequence that includes the amino acid sequences of (H1-A1) of the CDRH1, (H2-A1) of the CDRH2, and (H3-A1) of the CDRH3 and that has 80% or more identity to the amino acid sequence of Sequence ID No. 20, for example. The amino acid sequence of (H-A3) above may be an amino acid sequence that includes the amino acid sequences of (H1-A1) of the CDRH1, (H2-A1) of the CDRH2, and (H3-A1) of the CDRH3 and that consists of the amino acid sequence of Sequence ID No. 20 with deletion, substitution, insertion, and/or addition of one or several amino acids, for example.

The amino acid sequence of (L-A1) above is a sequence that includes the amino acid sequences of (L1-A1) of the CDRL1, (L2-A1) of the CDRL2, and (L3-A1) of the CDRL3, for example. The amino acid sequence of (L-A2) above may be an amino acid sequence that includes the amino acid sequences of (L1-A1) of the CDRL1, (L2-A1) of the CDRL2, and (L3-A1) of the CDRL3 and that has 80% or more identity to the amino acid sequence of Sequence ID No. 32, for example. The amino acid sequence of (L-A3) above may be an amino acid sequence that includes the amino acid sequences of (L1-A1) of the CDRL1, (L2-A1) of the CDRL2, and (L3-A1) of the CDRL3 and that consists of the amino acid sequence of Sequence ID No. 32 with deletion, substitution, insertion, and/or addition of one or several amino acids, for example.

In the antibody of the present invention, the heavy-chain variable region is (H-A1) above, and the light-chain variable region is (L-A1) above, for example. The antibody that includes this combination is also referred to as an “antibody H1-3M4E5L” hereinafter.

The “identity” as used for the polypeptide of the heavy-chain variable region and the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the heavy-chain variable region and the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (2)

The antibodies or the like of Combination (2) are also referred to as an antibody H1-K52 group, for example. In Combination (2) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LB) above includes the CDRL1, the CDRL2, and the CDRL3, the CDRL1 is a polypeptide that includes the amino acid sequence of (L1-B) below, the CDRL2 is a polypeptide that includes the amino acid sequence of (L2-B) below, and the CDRL3 is a polypeptide that includes the amino acid sequence of (L3-B) below.

(L1-B) An amino acid sequence of (L1-B1), (L1-B2), or (L1-B3) below:
(L1-B1) an amino acid sequence of Sequence ID No. 33 (QSISSY),
(L1-B2) an amino acid sequence having 80% or more identity to the amino acid sequence of Sequence ID No. 33, and
(L1-B3) an amino acid sequence consisting of the amino acid sequence of Sequence ID No. 33 with deletion, substitution, insertion, and/or addition of one or several amino acids.

(L2-B) An amino acid sequence of (L2-B1), (L2-B2), or (L2-B3) below:
(L2-B1) an amino acid sequence of Sequence ID No. 34 (AAS),
(L2-B2) an amino acid sequence having 80% or more identity to the amino acid sequence of Sequence ID No. 34, and
(L2-B3) an amino acid sequence consisting of the amino acid sequence of Sequence ID No. 34 with deletion, substitution, insertion, and/or addition of one or several amino acids.

(L3-B) An amino acid sequence of (L3-B1), (L3-B2), or (L3-B3) below:
(L3-B1) an amino acid sequence of Sequence ID No. 35 (QQYYSTPQT),
(L3-B2) an amino acid sequence having 80% or more identity to the amino acid sequence of Sequence ID No. 35, and
(L3-B3) an amino acid sequence consisting of the amino acid sequence of Sequence ID No. 35 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (2) above, the heavy-chain variable region of (HA) above includes a polypeptide consisting of the amino acid sequence of (H-A) above, for example. The light-chain variable region of (LB) above includes a polypeptide consisting of the amino acid sequence of (L-B) below, for example.

(L-B) An amino acid sequence of (L-B1), (L-B2), or (L-B3) below:
(L-B1) an amino acid sequence of Sequence ID No. 36:

Sequence ID No. 36:
DIQMTQSPSAMSASVGDRVTITCRASQSIS
SYLNWYQQKPGKAPKLLIYAASSLQSGVP
SRFSGSGSGTDFTLTISSLQPEDFATYYCQ
QYYSTPQTFGPGTKVDIK,

(L-B2) an amino acid sequence having 80% or more identity to the amino acid sequence of Sequence ID No. 36, and
(L-B3) an amino acid sequence consisting of the amino acid sequence of Sequence ID No. 36 with deletion, substitution, insertion, and/or addition of one or several amino acids.

The amino acid sequence of (L-B1) above is a sequence that includes the amino acid sequences of (L1-B1) of the CDRL1, (L2-B1) of the CDRL2, and (L3-B1) of the CDRL3, for example. The amino acid sequence of (L-B2) above may be an amino acid sequence that includes the amino acid sequences of (L1-B1) of the CDRL1, (L2-B1) of the CDRL2, and (L3-B1) of the CDRL3, and having 80% or more identity to the amino acid sequence of Sequence ID No. 36, for example. The amino acid sequence of (L-B3) above may be an amino acid sequence that includes the amino acid sequences of (L1-B1) of the CDRL1, (L2-B1) of the CDRL2, and (L3-B1) of the CDRL3, and consisting of the amino acid sequence of Sequence ID No. 36 with deletion, substitution, insertion, and/or addition of one or several amino acids, for example.

In the antibody of the present invention, the heavy-chain variable region is (H-A1) above, and the light-chain variable region is (L-B1) above, for example. The antibody that includes this combination is also referred to as an “antibody H1-K52” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (3)

The antibodies or the like of Combination (3) are also referred to as an antibody H1-K73 group, for example. In Combination (3) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LC) above includes the CDRL1, the CDRL2, and the CDRL3, the CDRL1 is a polypeptide that includes the amino acid sequence of (L1-C) below, the CDRL2 is a polypeptide that includes the amino acid sequence of (L2-C) below, and the CDRL3 is a polypeptide that includes the amino acid sequence of (L3-C) below.

(L1-C) An amino acid sequence of (L1-C1), (L1-C2), or (L1-C3) below:
(L1-C1) an amino acid sequence of Sequence ID No. 37 (QSISSY),
(L1-C2) an amino acid sequence having 80% or more identity to the amino acid sequence of Sequence ID No. 37, and
(L1-C3) an amino acid sequence consisting of the amino acid sequence of Sequence ID No. 37 with deletion, substitution, insertion, and/or addition of one or several amino acids.

(L2-C) An amino acid sequence of (L2-C1), (L2-C2), or (L2-C3) below:
(L2-C1) an amino acid sequence of Sequence ID No. 38 (AAS),
(L2-C2) an amino acid sequence having 80% or more identity to the amino acid sequence of Sequence ID No. 38, and
(L2-C3) an amino acid sequence consisting of the amino acid sequence of Sequence ID No. 38 with deletion, substitution, insertion, and/or addition of one or several amino acids.

(L3-C) An amino acid sequence of (L3-C1), (L3-C2), or (L3-C3) below:
(L3-C1) an amino acid sequence of Sequence ID No. 39 (QQYESYRRS),
(L3-C2) an amino acid sequence having 80% or more identity to the amino acid sequence of Sequence ID No. 39, and
(L3-C3) an amino acid sequence consisting of the amino acid sequence of Sequence ID No. 39 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (3) above, the heavy-chain variable region of (HA) above includes a polypeptide consisting of the amino acid sequence of (H-A) above, for example. The light-chain variable region of (LC) above includes a polypeptide consisting of the amino acid sequence of (L-C) below, for example.

(L-C) An amino acid sequence of (L-C1), (L-C2), or (L-C3) below:
(L-C1) an amino acid sequence of Sequence ID No. 40:

Sequence ID No. 40:
DIQMTQSPSSLSASVGDRVTITCRASQSIS
SYLNWYQQKAGKAPKLLIYAASSLQSGVPS
RFSGSGSGTDFTLTISCLOSEDVATYYCQQ
YESYRRSFGQGTKVEIK,

(L-C2) an amino acid sequence having 80% or more identity to the amino acid sequence of Sequence ID No. 40, and
(L-C3) an amino acid sequence consisting of the amino acid sequence of Sequence ID No. 40 with deletion, substitution, insertion, and/or addition of one or several amino acids.

The amino acid sequence of (L-C1) above is a sequence that includes the amino acid sequences of (L1-C1) of the CDRL1, (L2-C1) of the CDRL2, and (L3-C1) of the CDRL3, for example. The amino acid sequence of (L-C2) above may be an amino acid sequence that includes the amino acid sequences of (L1-C1) of the CDRL1, (L2-C1) of the CDRL2, and (L3-C1) of the CDRL3 and that has 80% or more identity to the amino acid sequence of Sequence ID No. 40, for example. The amino acid sequence of (L-C3) above may be an amino acid sequence that includes the amino acid sequences of (L1-C1) of the CDRL1, (L2-C1) of the CDRL2, and (L3-C1) of the CDRL3 and that consists of the amino acid sequence of Sequence ID No. 40 with deletion, substitution, insertion, and/or addition of one or several amino acids, for example.

In the antibody of the present invention, the heavy-chain variable region is (H-A1) above, and the light-chain variable region is (L-C1) above, for example. The antibody that includes this combination is also referred to as an “antibody H1-K73” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (4)

The antibodies or the like of Combination (4) are also referred to as an antibody H1-K121-K124 group, for example. In Combination (4) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LD) above includes the CDRL1, the CDRL2, and the CDRL3, the CDRL1 is a polypeptide that includes the amino acid sequence of (L1-D) below, the CDRL2 is a polypeptide that includes the amino acid sequence of (L2-D) below, and the CDRL3 is a polypeptide that includes the amino acid sequence of (L3-D) below.

(L1-D) An amino acid sequence of (L1-D1), (L1-D2), or (L1-D3) below:
(L1-D1) an amino acid sequence of Sequence ID No. 41 (QSISSY),
(L1-D2) an amino acid sequence having 80% or more identity to the amino acid sequence of Sequence ID No. 41, and
(L1-D3) an amino acid sequence consisting of the amino acid sequence of Sequence ID No. 41 with deletion, substitution, insertion, and/or addition of one or several amino acids.

(L2-D) An amino acid sequence of (L2-D1), (L2-D2), or (L2-D3) below:
(L2-D1) an amino acid sequence of Sequence ID No. 42 (AAS),
(L2-D2) an amino acid sequence having 80% or more identity to the amino acid sequence of Sequence ID No. 42, and
(L2-D3) an amino acid sequence consisting of the amino acid sequence of Sequence ID No. 42 with deletion, substitution, insertion, and/or addition of one or several amino acids.

(L3-D) An amino acid sequence of (L3-D1), (L3-D2), or (L3-D3) below:
(L3-D1) an amino acid sequence of Sequence ID No. 43 (QQYNSYSRT),
(L3-D2) an amino acid sequence having 80% or more identity to the amino acid sequence of Sequence ID No. 43, and
(L3-D3) an amino acid sequence consisting of the amino acid sequence of Sequence ID No. 43 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (4) above, the heavy-chain variable region of (HA) above includes a polypeptide consisting of the amino acid sequence of (H-A) above, for example. The light-chain variable region of (LD) above includes a polypeptide consisting of the amino acid sequence of (L-DA) or (L-DB) below, for example.

(L-DA) An amino acid sequence of (L-DA1), (L-DA2), or (L-DA3) below:
(L-DA1) an amino acid sequence of Sequence ID No. 44:

Sequence ID No. 44:
DIQMTQSPSSLSASVGDRVTITCRASQSIS
SYLNWYQQKPGKAPKLLIYAASSLQSGVPS
RFSGSGSGTDFTLTISCLOSEDFATYYCQQ
YNSYSRTFGQGTKVEIK

(L-DA2) an amino acid sequence having 80% or more identity to the amino acid sequence of Sequence ID No. 44, and
(L-DA3) an amino acid sequence consisting of the amino acid sequence of Sequence ID No. 44 with deletion, substitution, insertion, and/or addition of one or several amino acids.

(L-DB) An amino acid sequence of (L-DB1), (L-DB2), or (L-DB3) below:
(L-DB1) an amino acid sequence of Sequence ID No. 45:

Sequence ID No. 45:
DIQMTQSPSSLSASVGDRVTITCRASQSIS
SYLNWYQQKPGKAPKLLIYAASRLESGVPS
RFSGSGSGTDFTLTISCLOSEDFATYYCQQ
YNSYSRTFGQGTKVEIK,

(L-DB2) an amino acid sequence having 80% or more identity to the amino acid sequence of Sequence ID No. 45, and
(L-DB3) an amino acid sequence consisting of the amino acid sequence of Sequence ID No. 45 with deletion, substitution, insertion, and/or addition of one or several amino acids.

The amino acid sequence of (L-DA1) above is a sequence that includes the amino acid sequences of (L1-D1) of the CDRL1, (L2-D1) of the CDRL2, and (L3-D1) of the CDRL3, for example. The amino acid sequence of (L-DA2) above may be an amino acid sequence that includes the amino acid sequences of (L1-D1) of the CDRL1, (L2-D1) of the CDRL2, and (L3-D1) of the CDRL3 and that has 80% or more identity to the amino acid sequence of Sequence ID No. 44, for example. The amino acid sequence of (L-DA3) above may be an amino acid sequence that includes the amino acid sequences of (L1-D1) of the CDRL1, (L2-D1) of the CDRL2, and (L3-D1) of the CDRL3 and that consists of the amino acid sequence of Sequence ID No. 44 with deletion, substitution, insertion, and/or addition of one or several amino acids, for example.

The amino acid sequence of (L-DB1) above is a sequence that includes the amino acid sequences of (L1-D1) of the CDRL1, (L2-D1) of the CDRL2, and (L3-D1) of the CDRL3, for example. The amino acid sequence of (L-DB2) above may be an amino acid sequence that includes the amino acid sequences of (L1-D1) of the CDRL1, (L2-D1) of the CDRL2, and (L3-D1) of the CDRL3 and that has 80% or more identity to the amino acid sequence of Sequence ID No. 45, for example. The amino acid sequence of (L-DB3) above may be an amino acid sequence that includes the amino acid sequences of (L1-D1) of the CDRL1, (L2-D1) of the CDRL2, and (L3-D1) of the CDRL3 and that consists of the amino acid sequence of Sequence ID No. 45 with deletion, substitution, insertion, and/or addition of one or several amino acids, for example.

In the antibody of the present invention, the heavy-chain variable region is (H-A1) above, and the light-chain variable region is (L-DA1) above, for example. The antibody that includes this combination is also referred to as an “antibody H1-K121” hereinafter. In the antibody of the present invention, the heavy-chain variable region is (H-A1) above, and the light-chain variable region is (L-DB1) above, for example. The antibody that includes this combination is also referred to as an “antibody H1-K124” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (5)

The antibodies or the like of Combination (5) are also referred to as an antibody H1-K125 group, for example. In Combination (5) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LE) above includes the CDRL1, the CDRL2, and the CDRL3, the CDRL1 is a polypeptide that includes the amino acid sequence of (L1-E) below, the CDRL2 is a polypeptide that includes the amino acid sequence of (L2-E) below, and the CDRL3 is a polypeptide that includes the amino acid sequence of (L3-E) below.

(L1-E) An amino acid sequence of (L1-E1), (L1-E2), or (L1-E3) below:
(L1-E1) an amino acid sequence of Sequence ID No. 46 (QSISSY),
(L1-E2) an amino acid sequence having 80% or more identity to the amino acid sequence of Sequence ID No. 46, and
(L1-E3) an amino acid sequence consisting of the amino acid sequence of Sequence ID No. 46 with deletion, substitution, insertion, and/or addition of one or several amino acids.

(L2-E) An amino acid sequence of (L2-E1), (L2-E2), or (L2-E3) below:
(L2-E1) an amino acid sequence of Sequence ID No. 47 (AAS),
(L2-E2) an amino acid sequence having 80% or more identity to the amino acid sequence of Sequence ID No. 47, and
(L2-E3) an amino acid sequence consisting of the amino acid sequence of Sequence ID No. 47 with deletion, substitution, insertion, and/or addition of one or several amino acids.

(L3-E) An amino acid sequence of (L3-E1), (L3-E2), or (L3-E3) below:
(L3-E1) an amino acid sequence of Sequence ID No. 48 (QQYNSYSPCT),
(L3-E2) an amino acid sequence having 80% or more identity to the amino acid sequence of Sequence ID No. 48, and
(L3-E3) an amino acid sequence consisting of the amino acid sequence of Sequence ID No. 48 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (5) above, the heavy-chain variable region of (HA) above includes a polypeptide consisting of the amino acid sequence of (H-A) above, for example. The light-chain variable region of (LE) above includes a polypeptide consisting of the amino acid sequence of (L-E) below, for example.

(L-E) An amino acid sequence of (L-E1), (L-E2), or (L-E3) below:
(L-E1) an amino acid sequence of Sequence ID No. 49:

Sequence ID No. 49:
DIQMTQSPSSLSASVGDRVTITCRASQSIS
SYLNWYQQKPGKAPKLLLYAASRLESGVPS
RFSGSGSGTEFTLTISSLQPDDFATYYCQQ
YNSYSPCTFGPGTKVDIK,

(L-E2) an amino acid sequence having 80% or more identity to the amino acid sequence of Sequence ID No. 49, and
(L-E3) an amino acid sequence consisting of the amino acid sequence of Sequence ID No. 49 with deletion, substitution, insertion, and/or addition of one or several amino acids.

The amino acid sequence of (L-E1) above is a sequence that includes the amino acid sequences of (L1-E1) of the CDRL1, (L2-E1) of the CDRL2, and (L3-E1) of the CDRL3, for example. The amino acid sequence of (L-E2) above may be an amino acid sequence that includes the amino acid sequences of (L1-E1) of the CDRL1, (L2-E1) of the CDRL2, and (L3-E1) of the CDRL3, and that has 80% or more identity to the amino acid sequence of Sequence ID No. 49, for example. The amino acid sequence of (L-E3) above may be an amino acid sequence that includes the amino acid sequences of (L1-E1) of the CDRL1, (L2-E1) of the CDRL2, and (L3-E1) of the CDRL3 and that consists of the amino acid sequence of Sequence ID No. 49 with deletion, substitution, insertion, and/or addition of one or several amino acids, for example.

In the antibody of the present invention, the heavy-chain variable region is (H-A1) above, and the light-chain variable region is (L-E1) above, for example. The antibody that includes this combination is also referred to as an “antibody H1-K125” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (6)

The antibodies or the like of Combination (6) are also referred to as an antibody H1-K131 group, for example. In Combination (6) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LF) above includes the CDRL1, the CDRL2, and the CDRL3, the CDRL1 is a polypeptide that includes the amino acid sequence of (L1-F) below, the CDRL2 is a polypeptide that includes the amino acid sequence of (L2-F) below, and the CDRL3 is a polypeptide that includes the amino acid sequence of (L3-F) below.

(L1-F) An amino acid sequence of (L1-F1), (L1-F2), or (L1-F3) below:
(L1-F1) an amino acid sequence of Sequence ID No. 50 (QDISRY),
(L1-F2) an amino acid sequence having 80% or more identity to the amino acid sequence of Sequence ID No. 50, and
(L1-F3) an amino acid sequence consisting of the amino acid sequence of Sequence ID No. 50 with deletion, substitution, insertion, and/or addition of one or several amino acids.

(L2-F) An amino acid sequence of (L2-F1), (L2-F2), or (L2-F3) below:
(L2-F1) an amino acid sequence of Sequence ID No. 51 (AAS),
(L2-F2) an amino acid sequence having 80% or more identity to the amino acid sequence of Sequence ID No. 51, and
(L2-F3) an amino acid sequence consisting of the amino acid sequence of Sequence ID No. 51 with deletion, substitution, insertion, and/or addition of one or several amino acids.

(L3-F) An amino acid sequence of (L3-F1), (L3-F2), or (L3-F3) below:
(L3-F1) an amino acid sequence of Sequence ID No. 52 (QQYDNLIT),
(L3-F2) an amino acid sequence having 80% or more identity to the amino acid sequence of Sequence ID No. 52, and
(L3-F3) an amino acid sequence consisting of the amino acid sequence of Sequence ID No. 52 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (6) above, the heavy-chain variable region of (HA) above includes a polypeptide consisting of the amino acid sequence of (H-A) above, for example. The light-chain variable region of (LF) above includes a polypeptide consisting of the amino acid sequence of (L-F) below, for example.

(L-F) An amino acid sequence of (L-F1), (L-F2), or (L-F3) below:
(L-F1) an amino acid sequence of Sequence ID No. 53:

Sequence ID No. 53:
DIQMTQSPSSLSASVGDRVSITCRASQDIS
RYLNWYQQKPGKAPKLLLYAASRLESGVPS
RFSGSGSGTDFTLTITSLQPDDFATYYCQQ
YDNLITFGQGTRLEIK,

(L-F2) an amino acid sequence having 80% or more identity to the amino acid sequence of Sequence ID No. 53, and
(L-F3) an amino acid sequence consisting of the amino acid sequence of Sequence ID No. 53 with deletion, substitution, insertion, and/or addition of one or several amino acids.

The amino acid sequence of (L-F1) above is a sequence that includes the amino acid sequences of (L1-F1) of the CDRL1, (L2-F1) of the CDRL2, and (L3-F1) of the CDRL3, for example. The amino acid sequence of (L-F2) above may be an amino acid sequence that includes the amino acid sequences of (L1-F1) of the CDRL1, (L2-F1) of the CDRL2, and (L3-F1) of the CDRL3 and that has 80% or more identity to the amino acid sequence of Sequence ID No. 53, for example. The amino acid sequence of (L-F3) above may be an amino acid sequence that includes the amino acid sequences of (L1-F1) of the CDRL1, (L2-F1) of the CDRL2, and (L3-F1) of the CDRL3 and that consists of the amino acid sequence of Sequence ID No. 53 with deletion, substitution, insertion, and/or addition of one or several amino acids, for example.

In the antibody of the present invention, the heavy-chain variable region is (H-A1) above, and the light-chain variable region is (L-F1) above, for example. The antibody that includes this combination is also referred to as an “antibody H1-K131” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (7)

The antibodies or the like of Combination (7) are also referred to as an antibody H1-K145 group, for example. In Combination (7) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LG) above includes the CDRL1, the CDRL2, and the CDRL3, the CDRL1 is a polypeptide that includes the amino acid sequence of (L1-G) below, the CDRL2 is a polypeptide that includes the amino acid sequence of (L2-G) below, and the CDRL3 is a polypeptide that includes the amino acid sequence of (L3-G) below.

(L1-G) An amino acid sequence of (L1-G1), (L1-G2), or (L1-G3) below:
(L1-G1) an amino acid sequence of Sequence ID No. 54 (QDISRY),
(L1-G2) an amino acid sequence having 80% or more identity to the amino acid sequence of Sequence ID No. 54, and
(L1-G3) an amino acid sequence consisting of the amino acid sequence of Sequence ID No. 54 with deletion, substitution, insertion, and/or addition of one or several amino acids.

(L2-G) An amino acid sequence of (L2-G1), (L2-G2), or (L2-G3) below:
(L2-G1) an amino acid sequence of Sequence ID No. 55 (AAS),
(L2-G2) an amino acid sequence having 80% or more identity to the amino acid sequence of Sequence ID No. 55, and
(L2-G3) an amino acid sequence consisting of the amino acid sequence of Sequence ID No. 55 with deletion, substitution, insertion, and/or addition of one or several amino acids.

(L3-G) An amino acid sequence of (L3-G1), (L3-G2), or (L3-G3) below:
(L3-G1) an amino acid sequence of Sequence ID No. 56 (QQYNSYSRT),
(L3-G2) an amino acid sequence having 80% or more identity to the amino acid sequence of Sequence ID No. 56, and
(L3-G3) an amino acid sequence consisting of the amino acid sequence of Sequence ID No. 56 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (7) above, the heavy-chain variable region of (HA) above includes a polypeptide consisting of the amino acid sequence of (H-A) above, for example. The light-chain variable region of (LG) above includes a polypeptide consisting of the amino acid sequence of (L-G) below, for example.

(L-G) An amino acid sequence of (L-G1), (L-G2), or (L-G3) below:
(L-G1) an amino acid sequence of Sequence ID No. 57:

Sequence ID No. 57:
DIQMTQSPSSLSASVGDRVSITCRASQDIS
RYLNWYQQKPGKAPKLLLYAASRLESGVPS
RFSGSGSGTDFTLTITSLQPDDFATYYCQQ
YNSYSRTFGQGTKVEIK,

(L-G2) an amino acid sequence having 80% or more identity to the amino acid sequence of Sequence ID No. 57, and
(L-G3) an amino acid sequence consisting of the amino acid sequence of Sequence ID No. 57 with deletion, substitution, insertion, and/or addition of one or several amino acids.

The amino acid sequence of (L-G1) above is a sequence that includes the amino acid sequences of (L1-G1) of the CDRL1, (L2-G1) of the CDRL2, and (L3-G1) of the CDRL3, for example. The amino acid sequence of (L-G2) above may be an amino acid sequence that includes the amino acid sequences of (L1-G1) of the CDRL1, (L2-G1) of the CDRL2, and (L3-G1) of the CDRL3 and that has 80% or more identity to the amino acid sequence of Sequence ID No. 57, for example. The amino acid sequence of (L-G3) above may be an amino acid sequence that includes the amino acid sequences of (L1-G1) of the CDRL1, (L2-G1) of the CDRL2, and (L3-G1) of the CDRL3 and that consists of the amino acid sequence of Sequence ID No. 57 with deletion, substitution, insertion, and/or addition of one or several amino acids, for example.

In the antibody of the present invention, the heavy-chain variable region is (H-A1) above, and the light-chain variable region is (L-G1) above, for example. The antibody that includes this combination is also referred to as an “antibody H1-K145” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (8)

The antibodies or the like of Combination (8) are also referred to as an antibody H1-K151 group, for example. In Combination (8) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LH) above includes the CDRL1, the CDRL2, and the CDRL3, the CDRL1 is a polypeptide that includes the amino acid sequence of (L1-H) below, the CDRL2 is a polypeptide that includes the amino acid sequence of (L2-H) below, and the CDRL3 is a polypeptide that includes the amino acid sequence of (L3-H) below.

(L1-H) An amino acid sequence of (L1-H1), (L1-H2), or (L1-H3) below:
(L1-H1) an amino acid sequence of Sequence ID No. 58 (QSISSY),
(L1-H2) an amino acid sequence having 80% or more identity to the amino acid sequence of Sequence ID No. 58, and
(L1-H3) an amino acid sequence consisting of the amino acid sequence of Sequence ID No. 58 with deletion, substitution, insertion, and/or addition of one or several amino acids.

(L2-H) An amino acid sequence of (L2-H1), (L2-H2), or (L2-H3) below:
(L2-H1) an amino acid sequence of Sequence ID No. 59 (AAS),
(L2-H2) an amino acid sequence having 80% or more identity to the amino acid sequence of Sequence ID No. 59, and
(L2-H3) an amino acid sequence consisting of the amino acid sequence of Sequence ID No. 59 with deletion, substitution, insertion, and/or addition of one or several amino acids.

(L3-H) An amino acid sequence of (L3-H1), (L3-H2), or (L3-H3) below:
(L3-H1) an amino acid sequence of Sequence ID No. 60 (QQYDNLIT),
(L3-H2) an amino acid sequence having 80% or more identity to the amino acid sequence of Sequence ID No. 60, and
(L3-H3) an amino acid sequence consisting of the amino acid sequence of Sequence ID No. 60 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (8) above, the heavy-chain variable region of (HA) above includes a polypeptide consisting of the amino acid sequence of (H-A) above, for example. The light-chain variable region of (LH) above includes a polypeptide consisting of the amino acid sequence of (L-H) below, for example.

(L-H) An amino acid sequence of (L-H1), (L-H2), or (L-H3) below:
(L-H1) an amino acid sequence of Sequence ID No. 61:

Sequence ID No. 61:
DIQMTQSPSSLSASVGDRVTITCRASQSIS
SYLNWYQQKPGKAPKLLIYAASSLQSGVPS
RFSGSGSGTDFTLTITSLQPDDFATYYCQQ
YDNLITFGQGTRLEIK,

(L-H2) an amino acid sequence having 80% or more identity to the amino acid sequence of Sequence ID No. 61, and
(L-H3) an amino acid sequence consisting of the amino acid sequence of Sequence ID No. 61 with deletion, substitution, insertion, and/or addition of one or several amino acids.

The amino acid sequence of (L-H1) above is a sequence that includes the amino acid sequences of (L1-H1) of the CDRL1, (L2-H1) of the CDRL2, and (L3-H1) of the CDRL3, for example. The amino acid sequence of (L-H2) above may be an amino acid sequence that includes the amino acid sequences of (L1-H1) of the CDRL1, (L2-H1) of the CDRL2, and (L3-H1) of the CDRL3 and that has 80% or more identity to the amino acid sequence of Sequence ID No. 61, for example. The amino acid sequence of (L-H3) above may be an amino acid sequence that includes the amino acid sequences of (L1-H1) of the CDRL1, (L2-H1) of the CDRL2, and (L3-H1) of the CDRL3 and that consists of the amino acid sequence of Sequence ID No. 61 with deletion, substitution, insertion, and/or addition of one or several amino acids, for example.

In the antibody of the present invention, the heavy-chain variable region is (H-A1) above, and the light-chain variable region is (L-H1) above, for example. The antibody that includes this combination is also referred to as an “antibody H1-K151” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (9)

The antibodies or the like of Combination (9) are also referred to as an antibody H1-K160 group, for example. In Combination (9) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (L1) above includes the CDRL1, the CDRL2, and the CDRL3, the CDRL1 is a polypeptide that includes the amino acid sequence of (L1-I) below, the CDRL2 is a polypeptide that includes the amino acid sequence of (L2-I) below, and the CDRL3 is a polypeptide that includes the amino acid sequence of (L3-I) below.

(L1-I) An amino acid sequence of (L1-I1), (L1-I2), or (L1-I3) below:
(L1-I1) an amino acid sequence of Sequence ID No. 62 (QSVSSN),
(L1-I2) an amino acid sequence having 80% or more identity to the amino acid sequence of Sequence ID No. 62, and
(L1-I3) an amino acid sequence consisting of the amino acid sequence of Sequence ID No. 62 with deletion, substitution, insertion, and/or addition of one or several amino acids.

(L2-I) An amino acid sequence of (L2-I1), (L2-I2), or (L2-I3) below:
(L2-I1) an amino acid sequence of Sequence ID No. 63 (GAS),
(L2-I2) an amino acid sequence having 80% or more identity to the amino acid sequence of Sequence ID No. 63, and
(L2-I3) an amino acid sequence consisting of the amino acid sequence of Sequence ID No. 63 with deletion, substitution, insertion, and/or addition of one or several amino acids.

(L3-I) An amino acid sequence of (L3-I1), (L3-I2), or (L3-I3) below:
(L3-I1) an amino acid sequence of Sequence ID No. 64 (QQYNSYSRT),
(L3-I2) an amino acid sequence having 80% or more identity to the amino acid sequence of Sequence ID No. 64, and
(L3-I3) an amino acid sequence consisting of the amino acid sequence of Sequence ID No. 64 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (9) above, the heavy-chain variable region of (HA) above includes a polypeptide consisting of the amino acid sequence of (H-A) above, for example. The light-chain variable region of (LI) above includes a polypeptide consisting of the amino acid sequence of (L-I) below, for example.

(L-I) An amino acid sequence of (L-I1), (L-I2), or (L-I3) below:
(L-I1) an amino acid sequence of Sequence ID No. 65:

Sequence ID No. 65:
EIVLTQSPATLSVSPGERATLSCRASQSVS
SNLAWYQQKPGQAPRLLIYGASTRATGIPA
RFSGSGSGTEFTLTISRLEPEDFATYYCQQ
YNSYSRTFGQGTKVEIK,

(L-I2) an amino acid sequence having 80% or more identity to the amino acid sequence of Sequence ID No. 65, and
(L-I3) an amino acid sequence consisting of the amino acid sequence of Sequence ID No. 65 with deletion, substitution, insertion, and/or addition of one or several amino acids.

The amino acid sequence of (L-I1) above is a sequence that includes the amino acid sequences of (L1-I1) of the CDRL1, (L2-I1) of the CDRL2, and (L3-I1) of the CDRL3, for example. The amino acid sequence of (L-I2) above may be an amino acid sequence that includes the amino acid sequences of (L1-I1) of the CDRL1, (L2-I1) of the CDRL2, and (L3-I1) of the CDRL3 and that has 80% or more identity to the amino acid sequence of Sequence ID No. 65, for example. The amino acid sequence of (L-I3) above may be an amino acid sequence that includes the amino acid sequences of (L1-I1) of the CDRL1, (L2-I1) of the CDRL2, and (L3-I1) of the CDRL3 and that consists of the amino acid sequence of Sequence ID No. 65 with deletion, substitution, insertion, and/or addition of one or several amino acids, for example.

In the antibody of the present invention, the heavy-chain variable region is (H-A1) above, and the light-chain variable region is (L-I1) above, for example. The antibody that includes this combination is also referred to as an “antibody H1-K160” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (10)

The antibodies or the like of Combination (10) are also referred to as an antibody H1-K173 group, for example. In Combination (10) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LJ) above includes the CDRL1, the CDRL2, and the CDRL3, the CDRL1 is a polypeptide that includes the amino acid sequence of (L1-J) below, the CDRL2 is a polypeptide that includes the amino acid sequence of (L2-J) below, and the CDRL3 is a polypeptide that includes the amino acid sequence of (L3-J) below.

(L1-J) An amino acid sequence of (L1-J1), (L1-J2), or (L1-J3) below:
(L1-J1) an amino acid sequence of Sequence ID No. 66 (QSISSY),
(L1-J2) an amino acid sequence having 80% or more identity to the amino acid sequence of Sequence ID No. 66, and
(L1-J3) an amino acid sequence consisting of the amino acid sequence of Sequence ID No. 66 with deletion, substitution, insertion, and/or addition of one or several amino acids.

(L2-J) An amino acid sequence of (L2-J1), (L2-J2), or (L2-J3) below:
(L2-J1) an amino acid sequence of Sequence ID No. 67 (AAS),
(L2-J2) an amino acid sequence having 80% or more identity to the amino acid sequence of Sequence ID No. 67, and
(L2-J3) an amino acid sequence consisting of the amino acid sequence of Sequence ID No. 67 with deletion, substitution, insertion, and/or addition of one or several amino acids.

(L3-J) An amino acid sequence of (L3-J1), (L3-J2), or (L3-J3) below:
(L3-J1) an amino acid sequence of Sequence ID No. 68 (QQYESYSRT),
(L3-J2) an amino acid sequence having 80% or more identity to the amino acid sequence of Sequence ID No. 68, and
(L3-J3) an amino acid sequence consisting of the amino acid sequence of Sequence ID No. 68 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (10) above, the heavy-chain variable region of (HA) above includes a polypeptide consisting of the amino acid sequence of (H-A) above, for example. The light-chain variable region of (LJ) above includes a polypeptide consisting of the amino acid sequence of (L-J) below, for example.

(L-J) An amino acid sequence of (L-J1), (L-J2), or (L-J3) below:
(L-J1) an amino acid sequence of Sequence ID No. 69:

Sequence ID No. 69:
DIQMTQSPSSLSASVGDRVTITCRASQSIS
SYLNWYQQKAGKAPKLLIYAASSLQSGVPS
RFSGSGSGTDFTLTISCLOSEDVATYYCQQ
YESYSRTFGQGTKVEIK,

(L-J2) an amino acid sequence having 80% or more identity to the amino acid sequence of Sequence ID No. 69, and
(L-J3) an amino acid sequence consisting of the amino acid sequence of Sequence ID No. 69 with deletion, substitution, insertion, and/or addition of one or several amino acids.

The amino acid sequence of (L-J1) above is a sequence that includes the amino acid sequences of (L1-J1) of the CDRL1, (L2-J1) of the CDRL2, and (L3-J1) of the CDRL3, for example. The amino acid sequence of (L-J2) above may be an amino acid sequence that includes the amino acid sequences of (L1-J1) of the CDRL1, (L2-J1) of the CDRL2, and (L3-J1) of the CDRL3 and that has 80% or more identity to the amino acid sequence of Sequence ID No. 69, for example. The amino acid sequence of (L-J3) above may be an amino acid sequence that includes the amino acid sequences of (L1-J1) of the CDRL1, (L2-J1) of the CDRL2, and (L3-J1) of the CDRL3 and that consists of the amino acid sequence of Sequence ID No. 69 with deletion, substitution, insertion, and/or addition of one or several amino acids, for example.

In the antibody of the present invention, the heavy-chain variable region is (H-A1) above, and the light-chain variable region is (L-J1) above, for example. The antibody that includes this combination is also referred to as an “antibody H1-K173” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (11)

The antibodies or the like of Combination (11) are also referred to as an antibody 3M4E5H-L1 group, for example. In Combination (11) above, the heavy-chain variable region of (HB) above includes the CDRH1, the CDRH2, and the CDRH3, the CDRH1 is a polypeptide that includes the amino acid sequence of (H1-B) below, the CDRH2 is a polypeptide that includes the amino acid sequence of (H2-B) below, and the CDRH3 is a polypeptide that includes the amino acid sequence of (H3-B) below. The light-chain variable region of (LK) above includes the CDRL1, the CDRL2, and the CDRL3, the CDRL1 is a polypeptide that includes the amino acid sequence of (L1-K) below, the CDRL2 is a polypeptide that includes the amino acid sequence of (L2-K) below, and the CDRL3 is a polypeptide that includes the amino acid sequence of (L3-K) below.

(H1-B) An amino acid sequence of (H1-B1), (H1-B2), or (H1-B3) below:
(H1-B1) an amino acid sequence of Sequence ID No. 21 (GFTFSTYQ),
(H1-B2) an amino acid sequence having 80% or more identity to the amino acid sequence of Sequence ID No. 21, and
(H1-B3) an amino acid sequence consisting of the amino acid sequence of Sequence ID No. 21 with deletion, substitution, insertion, and/or addition of one or several amino acids.

(H2-B) An amino acid sequence of (H2-B1), (H2-B2), or (H2-B3) below:
(H2-B1) an amino acid sequence of Sequence ID No. 22 (IVSSGGST),
(H2-B2) an amino acid sequence having 80% or more identity to the amino acid sequence of Sequence ID No. 22, and
(H2-B3) an amino acid sequence consisting of the amino acid sequence of Sequence ID No. 22 with deletion, substitution, insertion, and/or addition of one or several amino acids.

(H3-B) An amino acid sequence of (H3-B1), (H3-B2), or (H3-B3) below:
(H3-B1) an amino acid sequence of Sequence ID No. 23 (AGELLPYYGMDV),
(H3-B2) an amino acid sequence having 80% or more identity to the amino acid sequence of Sequence ID No. 23, and
(H3-B3) an amino acid sequence consisting of the amino acid sequence of Sequence ID No. 23 with deletion, substitution, insertion, and/or addition of one or several amino acids.

(L1-K) An amino acid sequence of (L1-K1), (L1-K2), or (L1-K3) below:
(L1-K1) an amino acid sequence of Sequence ID No. 70 (SSDVGGYDF),
(L1-K2) an amino acid sequence having 80% or more identity to the amino acid sequence of Sequence ID No. 70, and
(L1-K3) an amino acid sequence consisting of the amino acid sequence of Sequence ID No. 70 with deletion, substitution, insertion, and/or addition of one or several amino acids.

(L2-K) An amino acid sequence of (L2-K1), (L2-K2), or (L2-K3) below:
(L2-K1) an amino acid sequence of Sequence ID No. 71 (DVN),
(L2-K2) an amino acid sequence having 80% or more identity to the amino acid sequence of Sequence ID No. 71, and
(L2-K3) an amino acid sequence consisting of the amino acid sequence of Sequence ID No. 71 with deletion, substitution, insertion, and/or addition of one or several amino acids.

(L3-K) An amino acid sequence of (L3-K1), (L3-K2), or (L3-K3) below:
(L3-K1) an amino acid sequence of Sequence ID No. 72 (SSYAGSNSV),
(L3-K2) an amino acid sequence having 80% or more identity to the amino acid sequence of Sequence ID No. 72, and
(L3-K3) an amino acid sequence consisting of the amino acid sequence of Sequence ID No. 72 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (11) above, the heavy-chain variable region of (HB) above includes a polypeptide consisting of the amino acid sequence of (H-B) below, for example. The light-chain variable region of (LK) above includes a polypeptide consisting of the amino acid sequence of (L-K) below, for example.

(H-B) An amino acid sequence of (H-B1), (H-B2), or (H-B3) below:
(H-B1) an amino acid sequence of Sequence ID No. 24:

Sequence ID No. 24:
EVQLLESGGGLVQPGGSLRLSCAASGFTFSTYQMSWVRQAPGKGLEWVS
GIVSSGGSTAYADSVKGRFTISRDNSKNTLYLQMNSLRAEDTAVYYCAG
ELLPYYGMDVWGQGTTVTVSS,

(H-B2) an amino acid sequence having 80% or more identity to the amino acid sequence of Sequence ID No. 24, and
(H-B3) an amino acid sequence consisting of the amino acid sequence of Sequence ID No. 24 with deletion, substitution, insertion, and/or addition of one or several amino acids.

(L-K) An amino acid sequence of (L-K1), (L-K2), or (L-K3) below:
(L-K1) an amino acid sequence of Sequence ID No. 73:

Sequence ID No. 73:
QSALTQPPSASGSPGQSVTISCTGTSSDVGGYDFVSWYQQHPGEAPKLL
VYDVNNRPSGVSNRFSGSKSGNTASLTISGLQAEDEGDYYCSSYAGSNS
VFGTGTKVTVL,

(L-K2) an amino acid sequence having 80% or more identity to the amino acid sequence of Sequence ID No. 73, and
(L-K3) an amino acid sequence consisting of the amino acid sequence of Sequence ID No. 73 with deletion, substitution, insertion, and/or addition of one or several amino acids.

The amino acid sequence of (H-B1) above is a sequence that includes the amino acid sequences of (H1-B1) of the CDRH1, (H2-B1) of the CDRH2, and (H3-B1) of the CDRH3, for example. The amino acid sequence of (H-B2) above may be an amino acid sequence that includes the amino acid sequences of (H1-B1) of the CDRH1, (H2-B1) of the CDRH2, and (H3-B1) of the CDRH3 and that has 80% or more identity to the amino acid sequence of Sequence ID No. 24, for example. The amino acid sequence of (H-B3) above may be an amino acid sequence that includes the amino acid sequences of (H1-B1) of the CDRH1, (H2-B1) of the CDRH2, and (H3-B1) of the CDRH3 and that consists of the amino acid sequence of Sequence ID No. 24 with deletion, substitution, insertion, and/or addition of one or several amino acids, for example.

The amino acid sequence of (L-K1) above is a sequence that includes the amino acid sequences of (L1-K1) of the CDRL1, (L2-K1) of the CDRL2, and (L3-K1) of the CDRL3, for example. The amino acid sequence of (L-K2) above may be an amino acid sequence that includes the amino acid sequences of (L1-K1) of the CDRL1, (L2-K1) of the CDRL2, and (L3-K1) of the CDRL3 and that has 80% or more identity to the amino acid sequence of Sequence ID No. 73, for example. The amino acid sequence of (L-K3) above may be an amino acid sequence that includes the amino acid sequences of (L1-K1) of the CDRL1, (L2-K1) of the CDRL2, and (L3-K1) of the CDRL3 and that consists of the amino acid sequence of Sequence ID No. 73 with deletion, substitution, insertion, and/or addition of one or several amino acids, for example.

In the antibody of the present invention, the heavy-chain variable region is (H-B1) above, and the light-chain variable region is (L-K1) above, for example. The antibody that includes this combination is also referred to as an “antibody 3M4E5H-L1” hereinafter.

The “identity” as used for the polypeptide of the heavy-chain variable region and the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the heavy-chain variable region and the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (12)

The antibodies or the like of Combination (12) are also referred to as an antibody 3M4E5H-L66 group, for example. In Combination (12) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LL) above includes the CDRL1, the CDRL2, and the CDRL3, the CDRL1 is a polypeptide that includes the amino acid sequence of (L1-L) below, the CDRL2 is a polypeptide that includes the amino acid sequence of (L2-L) below, and the CDRL3 is a polypeptide that includes the amino acid sequence of (L3-L) below.

(L1-L) An amino acid sequence of (L1-L1), (L1-L2), or (L1-L3) below:
(L1-L1) an amino acid sequence of Sequence ID No. 74 (SSDVGGYEF),
(L1-L2) an amino acid sequence having 80% or more identity to the amino acid sequence of Sequence ID No. 74, and
(L1-L3) an amino acid sequence consisting of the amino acid sequence of Sequence ID No. 74 with deletion, substitution, insertion, and/or addition of one or several amino acids.

(L2-L) An amino acid sequence of (L2-L1), (L2-L2), or (L2-L3) below:
(L2-L1) an amino acid sequence of Sequence ID No. 75 (DVI),
(L2-L2) an amino acid sequence having 80% or more identity to the amino acid sequence of Sequence ID No. 75, and
(L2-L3) an amino acid sequence consisting of the amino acid sequence of Sequence ID No. 75 with deletion, substitution, insertion, and/or addition of one or several amino acids.

(L3-L) An amino acid sequence of (L3-L1), (L3-L2), or (L3-L3) below:
(L3-L1) an amino acid sequence of Sequence ID No. 76 (SSYTSSSTYV),
(L3-L2) an amino acid sequence having 80% or more identity to the amino acid sequence of Sequence ID No. 76, and
(L3-L3) an amino acid sequence consisting of the amino acid sequence of Sequence ID No. 76 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (12) above, the heavy-chain variable region of (HB) above includes a polypeptide consisting of the amino acid sequence of (H-B) above, for example. The light-chain variable region of (LL) above includes a polypeptide consisting of the amino acid sequence of (L-L) below, for example.

(L-L) An amino acid sequence of (L-L1), (L-L2), or (L-L3) below:
(L-L1) an amino acid sequence of Sequence ID No. 77:

Sequence ID No. 77:
QSALTQPASVSGSPGQSITISCTGTSSDVGGYEFVSWYQQHPGSAPKLI
IYDVIERPFGVSYRFSASKSGNTASLTISGLQGEDEADYFCSSYTSSST
YVFGTGTKVTVL,

(L-L2) an amino acid sequence having 80% or more identity to the amino acid sequence of Sequence ID No. 77, and
(L-L3) an amino acid sequence consisting of the amino acid sequence of Sequence ID No. 77 with deletion, substitution, insertion, and/or addition of one or several amino acids.

The amino acid sequence of (L-L1) above is a sequence that includes the amino acid sequences of (L1-L1) of the CDRL1, (L2-L1) of the CDRL2, and (L3-L1) of the CDRL3, for example. The amino acid sequence of (L-L2) above may be an amino acid sequence that includes the amino acid sequences of (L1-L1) of the CDRL1, (L2-L1) of the CDRL2, and (L3-L1) of the CDRL3 and that has 80% or more identity to the amino acid sequence of Sequence ID No. 77, for example. The amino acid sequence of (L-L3) above may be an amino acid sequence that includes the amino acid sequences of (L1-L1) of the CDRL1, (L2-L1) of the CDRL2, and (L3-L1) of the CDRL3 and that consists of the amino acid sequence of Sequence ID No. 77 with deletion, substitution, insertion, and/or addition of one or several amino acids, for example.

In the antibody of the present invention, the heavy-chain variable region is (H-B1) above, and the light-chain variable region is (L-L1) above, for example. The antibody that includes this combination is also referred to as an “antibody 3M4E5H-L66” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (13)

The antibodies or the like of Combination (13) are also referred to as an antibody 3M4E5H-L73 group, for example. In Combination (13) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LM) above includes the CDRL1, the CDRL2, and the CDRL3, the CDRL1 is a polypeptide that includes the amino acid sequence of (L1-M) below, the CDRL2 is a polypeptide that includes the amino acid sequence of (L2-M) below, and the CDRL3 is a polypeptide that includes the amino acid sequence of (L3-M) below.

(L1-M) An amino acid sequence of (L1-M1), (L1-M2), or (L1-M3) below:
(L1-M1) an amino acid sequence of Sequence ID No. 78 (GSDVGAYDY),
(L1-M2) an amino acid sequence having 80% or more identity to the amino acid sequence of Sequence ID No. 78, and
(L1-M3) an amino acid sequence consisting of the amino acid sequence of Sequence ID No. 78 with deletion, substitution, insertion, and/or addition of one or several amino acids.

(L2-M) An amino acid sequence of (L2-M1), (L2-M2), or (L2-M3) below:
(L2-M1) an amino acid sequence of Sequence ID No. 79 (DVS),
(L2-M2) an amino acid sequence having 80% or more identity to the amino acid sequence of Sequence ID No. 79, and
(L2-M3) an amino acid sequence consisting of the amino acid sequence of Sequence ID No. 79 with deletion, substitution, insertion, and/or addition of one or several amino acids.

(L3-M) An amino acid sequence of (L3-M1), (L3-M2), or (L3-M3) below:
(L3-M1) an amino acid sequence of Sequence ID No. 80 (SSYSGSSTWV),
(L3-M2) an amino acid sequence having 80% or more identity to the amino acid sequence of Sequence ID No. 80, and
(L3-M3) an amino acid sequence consisting of the amino acid sequence of Sequence ID No. 80 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (13) above, the heavy-chain variable region of (HB) above includes a polypeptide consisting of the amino acid sequence of (H-B) above, for example. The light-chain variable region of (LM) above includes a polypeptide consisting of the amino acid sequence of (L-M) below, for example.

(L-M) An amino acid sequence of (L-M1), (L-M2), or (L-M3) below:
(L-M1) an amino acid sequence of Sequence ID No. 81:

Sequence ID No. 81:
QSALTQPASVSGSPGQSITISCTGTGSDVGAYDYVSWYQHHPGRAPRLI
IRDVSVRPSGVPDRFSGSKSGNTASLTISGLQAEDEADYYCSSYSGSST
WVFGGGTKLTVL,

(L-M2) an amino acid sequence having 80% or more identity to the amino acid sequence of Sequence ID No. 81, and
(L-M3) an amino acid sequence consisting of the amino acid sequence of Sequence ID No. 81 with deletion, substitution, insertion, and/or addition of one or several amino acids.

The amino acid sequence of (L-M1) above is a sequence that includes the amino acid sequences of (L1-M1) of the CDRL1, (L2-M1) of the CDRL2, and (L3-M1) of the CDRL3, for example. The amino acid sequence of (L-M2) above may be an amino acid sequence that includes the amino acid sequences of (L1-M1) of the CDRL1, (L2-M1) of the CDRL2, and (L3-M1) of the CDRL3 and that has 80% or more identity to the amino acid sequence of Sequence ID No. 81, for example. The amino acid sequence of (L-M3) above may be an amino acid sequence that includes the amino acid sequences of (L1-M1) of the CDRL1, (L2-M1) of the CDRL2, and (L3-M1) of the CDRL3 and that consists of the amino acid sequence of Sequence ID No. 81 with deletion, substitution, insertion, and/or addition of one or several amino acids, for example.

In the antibody of the present invention, the heavy-chain variable region is (H-B1) above, and the light-chain variable region is (L-M1) above, for example. The antibody that includes this combination is also referred to as an “antibody 3M4E5H-L73” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (14)

The antibodies or the like of Combination (14) are also referred to as an antibody 3M4E5H-L80 group, for example. In Combination (14) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LN) above includes the CDRL1, the CDRL2, and the CDRL3, the CDRL1 is a polypeptide that includes the amino acid sequence of (L1-N) below, the CDRL2 is a polypeptide that includes the amino acid sequence of (L2-N) below, and the CDRL3 is a polypeptide that includes the amino acid sequence of (L3-N) below.

(L1-N) An amino acid sequence of (L1-N₁), (L1-N₂), or (L1-N₃) below:
(L1-N₁) an amino acid sequence of Sequence ID No. 82 (SSDVGSYNL),
(L1-N₂) an amino acid sequence having 80% or more identity to the amino acid sequence of Sequence ID No. 82, and
(L1-N₃) an amino acid sequence consisting of the amino acid sequence of Sequence ID No. 82 with deletion, substitution, insertion, and/or addition of one or several amino acids.

(L2-N) An amino acid sequence of (L2-N1), (L2-N2), or (L2-N3) below:
(L2-N1) an amino acid sequence of Sequence ID No. 83 (DVS),
(L2-N2) an amino acid sequence having 80% or more identity to the amino acid sequence of Sequence ID No. 83, and
(L2-N3) an amino acid sequence consisting of the amino acid sequence of Sequence ID No. 83 with deletion, substitution, insertion, and/or addition of one or several amino acids.

(L3-N) An amino acid sequence of (L3-N1), (L3-N2), or (L3-N3) below:
(L3-N1) an amino acid sequence of Sequence ID No. 84 (SSYTSSSTFAV),
(L3-N2) an amino acid sequence having 80% or more identity to the amino acid sequence of Sequence ID No. 84, and
(L3-N3) an amino acid sequence consisting of the amino acid sequence of Sequence ID No. 84 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (14) above, the heavy-chain variable region of (HB) above includes a polypeptide consisting of the amino acid sequence of (H-B) above, for example. The light-chain variable region of (LN) above includes a polypeptide consisting of the amino acid sequence of (L-N) below, for example.

(L-N) An amino acid sequence of (L-N₁), (L-N₂), or (L-N₃) below:
(L-N₁) an amino acid sequence of Sequence ID No. 85:

Sequence ID No. 85:
QSALTQPASVSGSPGQSITISCTGTSSDVGSYNLVSWYQQHPGKAPKLM
IYDVSNRPSGVSYRFSGSKSGNTASLTISGLQAEDEADYYCSSYTSSST
FAVFGGGTQLTVL

(L-N₂) an amino acid sequence having 80% or more identity to the amino acid sequence of Sequence ID No. 85, and
(L-N₃) an amino acid sequence consisting of the amino acid sequence of Sequence ID No. 85 with deletion, substitution, insertion, and/or addition of one or several amino acids.

The amino acid sequence of (L-N₁) above is a sequence that includes the amino acid sequences of (L1-N1) of the CDRL1, (L2-N1) of the CDRL2, and (L3-N1) of the CDRL3, for example. The amino acid sequence of (L-N₂) above may be an amino acid sequence that includes the amino acid sequences of (L1-N1) of the CDRL1, (L2-N1) of the CDRL2, and (L3-N₁) of the CDRL3 and that has 80% or more identity to the amino acid sequence of Sequence ID No. 85, for example. The amino acid sequence of (L-N₃) above may be an amino acid sequence that includes the amino acid sequences of (L1-N1) of the CDRL1, (L2-N1) of the CDRL2, and (L3-N1) of the CDRL3 and that consists of the amino acid sequence of Sequence ID No. 85 with deletion, substitution, insertion, and/or addition of one or several amino acids, for example.

In the antibody of the present invention, the heavy-chain variable region is (H-B1) above, and the light-chain variable region is (L-N1) above, for example. The antibody that includes this combination is also referred to as an “antibody 3M4E5H-L80” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (15)

The antibodies or the like of Combination (15) are also referred to as an antibody 3M4E5H-L88 group, for example. In Combination (15) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LO) above includes the CDRL1, the CDRL2, and the CDRL3, the CDRL1 is a polypeptide that includes the amino acid sequence of (L1-O) below, the CDRL2 is a polypeptide that includes the amino acid sequence of (L2-O) below, and the CDRL3 is a polypeptide that includes the amino acid sequence of (L3-O) below.

(L1-O) An amino acid sequence of (L1-O1), (L1-O2), or (L1-O3) below:
(L1-O1) an amino acid sequence of Sequence ID No. 86 (SSDVGGYNY),
(L1-O2) an amino acid sequence having 80% or more identity to the amino acid sequence of Sequence ID No. 86, and
(L1-O3) an amino acid sequence consisting of the amino acid sequence of Sequence ID No. 86 with deletion, substitution, insertion, and/or addition of one or several amino acids.

(L2-O) An amino acid sequence of (L2-O1), (L2-O2), or (L2-O3) below:
(L2-O1) an amino acid sequence of Sequence ID No. 87 (DVS),
(L2-O2) an amino acid sequence having 80% or more identity to the amino acid sequence of Sequence ID No. 87, and
(L2-O3) an amino acid sequence consisting of the amino acid sequence of Sequence ID No. 87 with deletion, substitution, insertion, and/or addition of one or several amino acids.

(L3-O) An amino acid sequence of (L3-O1), (L3-O2), or (L3-O3) below:
(L3-O1) an amino acid sequence of Sequence ID No. 88 (CSYAGGYYV),
(L3-O2) an amino acid sequence having 80% or more identity to the amino acid sequence of Sequence ID No. 88, and
(L3-O3) an amino acid sequence consisting of the amino acid sequence of Sequence ID No. 88 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (15) above, the heavy-chain variable region of (HB) above includes a polypeptide consisting of the amino acid sequence of (H-B) above, for example. The light-chain variable region of (LO) above includes a polypeptide consisting of the amino acid sequence of (L-O) below, for example.

(L-O) An amino acid sequence of (L-O1), (L-O2), or (L-O3) below:
(L-O1) an amino acid sequence of Sequence ID No. 89:

Sequence ID No. 89:
QSALPQPASVSGSPGQSVTISCTGTSSDVGGYNYVSWYQQHPGKAPKLM
IYDVSKRPSGVPDRFSGSKSGNTASLTVSGLQAEDEADYFCCSYAGGYY
VFGTGTKLTVL,

(L-O2) an amino acid sequence having 80% or more identity to the amino acid sequence of Sequence ID No. 89, and
(L-O3) an amino acid sequence consisting of the amino acid sequence of Sequence ID No. 89 with deletion, substitution, insertion, and/or addition of one or several amino acids.

The amino acid sequence of (L-O1) above is a sequence that includes the amino acid sequences of (L1-O1) of the CDRL1, (L2-O1) of the CDRL2, and (L3-O1) of the CDRL3, for example. The amino acid sequence of (L-O2) above may be an amino acid sequence that includes the amino acid sequences of (L1-O1) of the CDRL1, (L2-O1) of the CDRL2, and (L3-O1) of the CDRL3 and that has 80% or more identity to the amino acid sequence of Sequence ID No. 89, for example. The amino acid sequence of (L-O3) above may be an amino acid sequence that includes the amino acid sequences of (L1-O1) of the CDRL1, (L2-O1) of the CDRL2, and (L3-O1) of the CDRL3 and that consists of the amino acid sequence of Sequence ID No. 89 with deletion, substitution, insertion, and/or addition of one or several amino acids, for example.

In the antibody of the present invention, the heavy-chain variable region is (H-B1) above, and the light-chain variable region is (L-O1) above, for example. The antibody that includes this combination is also referred to as an “antibody 3M4E5H-L88” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (16)

The antibodies or the like of Combination (16) are also referred to as an antibody 3M4E5H-L102 group, for example. In Combination (16) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LP) above includes the CDRL1, the CDRL2, and the CDRL3, the CDRL1 is a polypeptide that includes the amino acid sequence of (L1-P) below, the CDRL2 is a polypeptide that includes the amino acid sequence of (L2-P) below, and the CDRL3 is a polypeptide that includes the amino acid sequence of (L3-P) below.

(L1-P) An amino acid sequence of (L1-P1), (L1-P2), or (L1-P3) below:
(L1-P1) an amino acid sequence of Sequence ID No. 90 (SSDVGGYNY),
(L1-P2) an amino acid sequence having 80% or more identity to the amino acid sequence of Sequence ID No. 90, and
(L1-P3) an amino acid sequence consisting of the amino acid sequence of Sequence ID No. 90 with deletion, substitution, insertion, and/or addition of one or several amino acids.

(L2-P) An amino acid sequence of (L2-P1), (L2-P2), or (L2-P3) below:
(L2-P1) an amino acid sequence of Sequence ID No. 91 (DVS),
(L2-P2) an amino acid sequence having 80% or more identity to the amino acid sequence of Sequence ID No. 91, and
(L2-P3) an amino acid sequence consisting of the amino acid sequence of Sequence ID No. 91 with deletion, substitution, insertion, and/or addition of one or several amino acids.

(L3-P) An amino acid sequence of (L3-P1), (L3-P2), or (L3-P3) below:
(L3-P1) an amino acid sequence of Sequence ID No. 92 (SSYAGSGSTPFV),
(L3-P2) an amino acid sequence having 80% or more identity to the amino acid sequence of Sequence ID No. 92, and
(L3-P3) an amino acid sequence consisting of the amino acid sequence of Sequence ID No. 92 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (16) above, the heavy-chain variable region of (HB) above includes a polypeptide consisting of the amino acid sequence of (H-B) above, for example. The light-chain variable region of (LP) above includes a polypeptide consisting of the amino acid sequence of (L-P) below, for example.

(L-P) An amino acid sequence of (L-P1), (L-P2), or (L-P3) below:
(L-P1) an amino acid sequence of Sequence ID No. 93:

Sequence ID No. 93:
QSALTQPPSASGSPGQSVTISCTGTSSDVGGYNYVSWYQQHPGKAPKLM
IYDVSKRPSGVPDRFSGSKSGNTASLTISGLQTEDEADYYCSSYAGSGS
TPFVFGTGTKLTVL,

(L-P2) an amino acid sequence having 80% or more identity to the amino acid sequence of Sequence ID No. 93, and
(L-P3) an amino acid sequence consisting of the amino acid sequence of Sequence ID No. 93 with deletion, substitution, insertion, and/or addition of one or several amino acids.

The amino acid sequence of (L-P1) above is a sequence that includes the amino acid sequences of (L1-P1) of the CDRL1, (L2-P1) of the CDRL2, and (L3-P1) of the CDRL3, for example. The amino acid sequence of (L-P2) above may be an amino acid sequence that includes the amino acid sequences of (L1-P1) of the CDRL1, (L2-P1) of the CDRL2, and (L3-P1) of the CDRL3 and that has 80% or more identity to the amino acid sequence of Sequence ID No. 93, for example. The amino acid sequence of (L-P3) above may be an amino acid sequence that includes the amino acid sequences of (L1-P1) of the CDRL1, (L2-P1) of the CDRL2, and (L3-P1) of the CDRL3 and that consists of the amino acid sequence of Sequence ID No. 93 with deletion, substitution, insertion, and/or addition of one or several amino acids, for example.

In the antibody of the present invention, the heavy-chain variable region is (H-B1) above, and the light-chain variable region is (L-P1) above, for example. The antibody that includes this combination is also referred to as an “antibody 3M4E5H-L102” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (17)

The antibodies or the like of Combination (17) are also referred to as an antibody 3M4E5H-L124 group, for example. In Combination (17) above, the heavy-chain variable region of (HB) above includes the CDRH1, the CDRH2, and the CDRH3, the CDRH1 is a polypeptide that includes the amino acid sequence of (H1-B) above, the CDRH2 is a polypeptide that includes the amino acid sequence of (H2-B) above, and the CDRH3 is a polypeptide that includes the amino acid sequence of (H3-B) above. The light-chain variable region of (LQ) above includes the CDRL1, the CDRL2, and the CDRL3, the CDRL1 is a polypeptide that includes the amino acid sequence of (L1-Q) below, the CDRL2 is a polypeptide that includes the amino acid sequence of (L2-Q) below, and the CDRL3 is a polypeptide that includes the amino acid sequence of (L3-Q) below.

(L1-Q) An amino acid sequence of (L1-Q1), (L1-Q2), or (L1-Q3) below:
(L1-Q1) an amino acid sequence of Sequence ID No. 94 (SSDVGGYNY),
(L1-Q2) an amino acid sequence having 80% or more identity to the amino acid sequence of Sequence ID No. 94, and
(L1-Q3) an amino acid sequence consisting of the amino acid sequence of Sequence ID No. 94 with deletion, substitution, insertion, and/or addition of one or several amino acids.

(L2-Q) An amino acid sequence of (L2-Q1), (L2-Q2), or (L2-Q3) below:
(L2-Q1) an amino acid sequence of Sequence ID No. 95 (DVS),
(L2-Q2) an amino acid sequence having 80% or more identity to the amino acid sequence of Sequence ID No. 95, and
(L2-Q3) an amino acid sequence consisting of the amino acid sequence of Sequence ID No. 95 with deletion, substitution, insertion, and/or addition of one or several amino acids.

(L3-Q) An amino acid sequence of (L3-Q1), (L3-Q2), or (L3-Q3) below:
(L3-Q1) an amino acid sequence of Sequence ID No. 96 (CSYAGRRYV),
(L3-Q2) an amino acid sequence having 80% or more identity to the amino acid sequence of Sequence ID No. 96, and
(L3-Q3) an amino acid sequence consisting of the amino acid sequence of Sequence ID No. 96 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (17) above, the heavy-chain variable region of (HB) above includes a polypeptide consisting of the amino acid sequence of (H-B) above, for example. The light-chain variable region of (LQ) above includes a polypeptide consisting of the amino acid sequence of (L-Q) below, for example.

(L-Q) An amino acid sequence of (L-Q1), (L-Q2), or (L-Q3) below:
(L-Q1) an amino acid sequence of Sequence ID No. 97:

Sequence ID No. 97:
QSALTQPASVSGSPGQSITISCTGTSSDVGGYNYVSWYQQHPGKAPKLM
IYDVSNRPSRVPDRFAGSKSGNTASLTISGLQAEDEADYYCCSYAGRRY
VFGTGTKLTVL,

(L-Q2) an amino acid sequence having 80% or more identity to the amino acid sequence of Sequence ID No. 97, and
(L-Q3) an amino acid sequence consisting of the amino acid sequence of Sequence ID No. 97 with deletion, substitution, insertion, and/or addition of one or several amino acids.

The amino acid sequence of (L-Q1) above is a sequence that includes the amino acid sequences of (L1-Q1) of the CDRL1, (L2-Q1) of the CDRL2, and (L3-Q1) of the CDRL3, for example. The amino acid sequence of (L-Q2) above may be an amino acid sequence that includes the amino acid sequences of (L1-Q1) of the CDRL1, (L2-Q1) of the CDRL2, and (L3-Q1) of the CDRL3 and that has 80% or more identity to the amino acid sequence of Sequence ID No. 97, for example. The amino acid sequence of (L-Q3) above may be an amino acid sequence that includes the amino acid sequences of (L1-Q1) of the CDRL1, (L2-Q1) of the CDRL2, and (L3-Q1) of the CDRL3 and that consists of the amino acid sequence of Sequence ID No. 97 with deletion, substitution, insertion, and/or addition of one or several amino acids, for example.

In the antibody of the present invention, the heavy-chain variable region is (H-B1) above, and the light-chain variable region is (L-Q1) above, for example. The antibody that includes this combination is also referred to as an “antibody 3M4E5H-L124” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination (18)

The antibodies or the like of Combination (18) are also referred to as an antibody H73-3M4E5L group, for example. In Combination (18) above, the heavy-chain variable region of (HC) above includes the CDRH1, the CDRH2, and the CDRH3, the CDRH1 is a polypeptide that includes the amino acid sequence of (H1-C) below, the CDRH2 is a polypeptide that includes the amino acid sequence of (H2-C) below, and the CDRH3 is a polypeptide that includes the amino acid sequence of (H3-C) below. The light-chain variable region of (LA) above includes the CDRL1, the CDRL2, and the CDRL3, the CDRL1 is a polypeptide that includes the amino acid sequence of (L1-A) above, the CDRL2 is a polypeptide that includes the amino acid sequence of (L2-A) above, and the CDRL3 is a polypeptide that includes the amino acid sequence of (L3-A) above.

(H1-C) An amino acid sequence of (H1-C1), (H1-C2), or (H1-C3) below:
(H1-C1) an amino acid sequence of Sequence ID No. 25 (GGSISSYY),
(H1-C2) an amino acid sequence having 80% or more identity to the amino acid sequence of Sequence ID No. 25, and
(H1-C3) an amino acid sequence consisting of the amino acid sequence of Sequence ID No. 25 with deletion, substitution, insertion, and/or addition of one or several amino acids.

(H2-C) An amino acid sequence of (H2-C1), (H2-C2), or (H2-C3) below:
(H2-C1) an amino acid sequence of Sequence ID No. 26 (INHSGST),
(H2-C2) an amino acid sequence having 80% or more identity to the amino acid sequence of Sequence ID No. 26, and
(H2-C3) an amino acid sequence consisting of the amino acid sequence of Sequence ID No. 26 with deletion, substitution, insertion, and/or addition of one or several amino acids.

(H3-C) An amino acid sequence of (H3-C1), (H3-C2), or (H3-C3) below:
(H3-C1) an amino acid sequence of Sequence ID No. 27 (ARCPIYYYGMDV),
(H3-C2) an amino acid sequence having 80% or more identity to the amino acid sequence of Sequence ID No. 27, and
(H3-C3) an amino acid sequence consisting of the amino acid sequence of Sequence ID No. 27 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (18) above, the heavy-chain variable region of (HC) above includes a polypeptide consisting of the amino acid sequence of (H-C) below, for example. The light-chain variable region of (LA) above includes a polypeptide consisting of the amino acid sequence of (L-A) above, for example.

(H-C) An amino acid sequence of (H-C1), (H-C2), or (H-C3) below:
(H-C1) an amino acid sequence of Sequence ID No. 28:

Sequence ID No. 28:
QLQLQESGPGLVKPSQTLSLTCTVSGGSISSYYWSWIRQPPGKGLEWIG
EINHSGSTNYNPSLKSRVTISVDTSKNQFSLKLSSVTAADTAVYYCARC
PIYYYGMDVWGQGTTVTVSS,

(H-C2) an amino acid sequence having 80% or more identity to the amino acid sequence of Sequence ID No. 28, and
(H-C3) an amino acid sequence consisting of the amino acid sequence of Sequence ID No. 28 with deletion, substitution, insertion, and/or addition of one or several amino acids.

The amino acid sequence of (H-C1) above is a sequence that includes the amino acid sequences of (H1-C1) of the CDRH1, (H2-C1) of the CDRH2, and (H3-C1) of the CDRH3, for example. The amino acid sequence of (H-C2) above may be an amino acid sequence that includes the amino acid sequences of (H1-C1) of the CDRH1, (H2-C1) of the CDRH2, and (H3-C1) of the CDRH3 and that has 80% or more identity to the amino acid sequence of Sequence ID No. 28, for example. The amino acid sequence of (H-C3) above may be an amino acid sequence that includes the amino acid sequences of (H1-C1) of the CDRH1, (H2-C1) of the CDRH2, and (H3-C1) of the CDRH3 and that consists of the amino acid sequence of Sequence ID No. 28 with deletion, substitution, insertion, and/or addition of one or several amino acids, for example.

In the antibody of the present invention, the heavy-chain variable region is (H-C1) above, and the light-chain variable region is (L-A1) above, for example. The antibody that includes this combination is also referred to as an “antibody H73-3M4E5L” hereinafter.

The “identity” as used for the polypeptide of the heavy-chain variable region refers to 80% or more, 85% or more, 90% or more, 95% or more, 96% or more, 97% or more, 98% or more, or 99% or more identity, for example.

The term “one or several” as used for the polypeptide of the heavy-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

In the present invention, the amino acid sequences of Sequence ID Nos. 17 to 97 are human-derived amino acid sequences, for example.

The binding of the antibody of the present invention or the like to A2/NY-ESO-1₁₅₇ can be confirmed using a technique for detecting the binding of an antibody to an antigen such as surface plasmon resonance (SPR) or flow cytometry, for example.

The antibody of the present invention or the like may further include a labeling substance, for example. There is no particular limitation on the labeling substance, and examples thereof include fluorescent substances, dyes, isotopes, and enzymes. Examples of the fluorescent substances include fluorophores such as pyrene, TAMRA, fluorescein, Cy3 dyes, Cy5 dyes, FAM dyes, rhodamine dyes, Texas red dyes, JOE, MAX, HEX, and TYE, and examples of the dyes include Alexa dyes such as Alexa488 and Alexa647. The antibody of the present invention or the like may be modified by a low-molecular compound such as a pharmaceutical compound. Examples of the low-molecular compound include anticancer agents such as tubulin inhibitors. The antibody of the present invention or the like is modified directly or indirectly by the low-molecular compound, for example. In the latter case, the antibody of the present invention or the like is modified by the low-molecular compound via a linker, for example.

The antibody of the present invention or the like may be immobilized on a carrier, a porous body, or the like, for example. There is no particular limitation on the carrier, and examples thereof include a substrate, a bead, and a container. Examples of the container include a microplate and a tube.

There is no particular limitation on a method for manufacturing the antibody of the present invention or the like, and the antibody or the like can be manufactured using genetic engineering techniques based on the amino acid sequence information described above, for example. Specifically, the antibody or the like can be manufactured as follows, for example. It should be noted that the present invention is not limited to this example.

First, a vector that includes nucleic acid sequences coding for the amino acid sequences of the above-mentioned regions, heavy chain, and/or light chain in the antibody of the present invention or the like is introduced into a host, and thus a transformant is obtained. Then, the transformant is cultured, a fraction containing an antibody capable of binding to A2/NY-ESO-1₁₅₇ is collected, and the antibody is isolated or purified from the obtained collected fraction.

Examples of the vector include a vector that includes a nucleic acid sequence coding for the heavy-chain variable region, a vector that includes a nucleic acid sequence coding for the light-chain variable region, a vector that includes a nucleic acid sequence coding for the heavy chain, and a vector that includes a nucleic acid sequence coding for the light chain. There is no particular limitation on the host as long as the vector can be introduced into the host, and the nucleic acid sequence in the vector can be expressed in the host. Examples of the host include mammalian cells such as HEK cells, CHO cells, COS cells, NSO cells, and SP2/0 cells. There is no particular limitation on a method for introducing the vector into a host, and a known method can be employed.

There is no particular limitation on a method for culturing the transformant, and it is possible to determine as appropriate which method is employed, in accordance with the type of host. The fraction containing an antibody can be collected as a liquid fraction after the cultured transformant is lysed, for example. There is no particular limitation on a method for isolating or purifying the antibody, and a known method can be employed.

In the present invention, the antibody is a monoclonal antibody, for example. Examples of the monoclonal antibody include monoclonal antibodies obtained through immunization of an animal, chimeric antibodies, humanized antibodies, and human antibodies (also referred to as “fully human antibodies”).

The chimeric antibody is an antibody formed by coupling a variable region of an antibody derived from a non-human animal and a constant region of a human antibody. The chimeric antibody can be produced as follows, for example. First, the gene of the variable region (V region) of a monoclonal antibody derived from a non-human animal capable of binding to the A2/NY-ESO-1₁₅₇ protein is prepared, the gene of the variable region and the gene of the constant region (C region) of a human antibody are coupled to each other, and then the resultant product is coupled to an expression vector. Cells transfected with the expression vector are cultured, and the chimeric antibody secreted into the culture medium is collected. The chimeric antibody can thus be prepared. There is no particular limitation on the animal from which the variable region is derived, and examples thereof include a rat and a mouse. The method for manufacturing a chimeric antibody is not limited to the above-mentioned method, and a chimeric antibody can be manufactured with reference to a known method such as the method disclosed in JP H3-73280B, for example.

The humanized antibody is an antibody in which only the CDRs are derived from a non-human animal and the other regions are derived from a human. The humanized antibody can be manufactured as follows, for example. First, the genes of the CDRs of a monoclonal antibody derived from a non-human animal are prepared and grafted into a gene of a human antibody (e.g., a region that corresponds to the constant region) (CDR grafting), and then the resultant product is coupled to an expression vector. Cells transfected with the expression vector are cultured, and a humanized antibody into which the target CDRs are grafted is secreted into the culture medium, and is then collected. The humanized antibody can thus be prepared. There is no particular limitation on the animal from which the CDRs are derived, and examples thereof include a rat and a mouse. The method for manufacturing a humanized antibody is not limited to the above-mentioned method, and a humanized antibody can be manufactured with reference to a known method such as the method disclosed in JP H4-506458A or the method disclosed in JP S62-296890A, for example.

The human antibody is an antibody in which the entire region is derived from a human. The human antibody can be produced by introducing the gene of a human antibody into a non-human animal, for example. Examples of an animal into which the gene of a human antibody is to be introduced include transgenic animals for producing a human antibody. There is no particular limitation on the type of animal, and an example thereof is a mouse. The human antibody can be manufactured with reference to known methods disclosed in Nature Genetics, Vol. 7, p. 13-21, 1994; Nature Genetics, Vol. 15, p. 146-156, 1997; JP H4-504365A; JP H7-509137A; WO 94/25585; Nature, Vol. 368, p. 856-859, 1994; JP H6-500233A; and the like, for example. The human antibody can also be manufactured using a phage display technique, for example, and can be manufactured with reference to a known method disclosed in Marks, J. D. et al.: J. Mol. Biol., Vol. 222, p. 581-597, 1991, or the like, for example.

The antibody of the present invention or the like can also be prepared through immunization of an animal with an antigen, for example. An example of the antigen is the A2/NY-ESO-1₁₅₇ protein. It is preferable to repeat the immunization with the antigen a plurality of times. The peptide fragment may be a peptide fragment constituted by only an antigenic determinant (epitope) or a peptide fragment that includes the antigenic determinant, for example.

The monoclonal antibody obtained through immunization of an animal can be manufactured with reference to known methods such as the methods disclosed in “Current Protocols in Molecular Biology” (John Wiley & Sons (1987)), Antibodies: A Laboratory Manual, Ed. Harlow and David Lane, Cold Spring Harbor Laboratory (1988)), and the like, for example. Specifically, an animal is immunized with an antigen, and antibody-producing cells collected from the immunized animal and myeloma cells lacking antibody producibility are fused to produce hybridomas, for example. Subsequently, antibody-producing cells are screened from the hybridomas, and a monoclonal hybridoma is produced through cloning. Then, this hybridoma clone is administered to an animal, and a monoclonal antibody obtained from the abdominal cavity is purified. Alternatively, the hybridoma is cultured, and then a monoclonal antibody is purified from the hybridoma culture solution. Producing the hybridoma clone in this manner makes it possible to stably supply monoclonal antibodies having uniform specificity.

It is preferable that the myeloma cells are derived from a mouse, a rat, a human, or the like, for example. The myeloma cells and the antibody-producing cells may be derived from the same species or different species, for example, and are preferably derived from the same species.

In the first antibody of the present invention or the like, the heavy-chain variable region of (H) above and the light-chain variable region of (L) above may be a heavy-chain variable region of (H) below and a light-chain variable region of (L) below.

(H) A heavy-chain variable region that includes
a heavy-chain complementarity determining region (CDRH) 1, a CDRH2, and a CDRH3,
wherein the CDRH1 is a polypeptide that includes an amino acid sequence of (H1),
the CDRH2 is a polypeptide that includes an amino acid sequence of (H2),
the CDRH3 is a polypeptide that includes an amino acid sequence of (H3),
the amino acid sequences of (H1), (H2), and (H3) are as follows:
(H1) an amino acid sequence of (H1-1), (H1-2), or (H1-3) below:
   (H1-1) any one of amino acid sequences of CDRH1 shown in Condition (H1) below,
   (H1-2) an amino acid sequence having 80% or more identity to the amino acid sequence of (H1-1), and
   (H1-3) an amino acid sequence consisting of the amino acid sequence of (H1-1) with deletion, substitution, insertion, and/or addition of one or several amino acids;
(H2) an amino acid sequence of (H2-1), (H2-2), or (H2-3) below:
   (H2-1) any one of amino acid sequences of CDRH2 shown in Condition (H1) below,
   (H2-2) an amino acid sequence having 80% or more identity to the amino acid sequence of (H2-1), and
   (H2-3) an amino acid sequence consisting of the amino acid sequence of (H2-1) with deletion, substitution, insertion, and/or addition of one or several amino acids; and
(H3) an amino acid sequence of (H3-1), (H3-2), or (H3-3) below:
   (H3-1) any one of amino acid sequences of CDRH3 shown in Condition (H1) below,
   (H3-2) an amino acid sequence having 80% or more identity to the amino acid sequence of (H3-1), and
   (H3-3) an amino acid sequence consisting of the amino acid sequence of (H3-1) with deletion, substitution, insertion, and/or addition of one or several amino acids, and

Condition (H1) is as follows:
the CDRH1 is GX₁X₂X₃SX₄X₅X₆ (Sequence ID No. 322),
the CDRH2 is X₇X₈X₉X₁₀SX₁₁GST (Sequence ID No. 323),
the CDRH3 is AX₁₂X₁₃X₁₄X₁₅X₁₆GMDV (Sequence ID No. 324),
X₁ is G or F,
X₂ is S or T,
X₃ is I or F,
X₄ is S or T,
X₅ is N or Y,
X₆ is Y or Q,
X₇ is not present or is I,
X₈ is V or N,
X₉ is S or H,
X₁₀ is not present or is Y,
X₁₁ is not present or is G,
X₁₂ is R or G,
X₁₃ is E or C,
X₁₄ is S, L, or P,
X₁₅ is Y, L, or I, and
X₁₆ is Y or P.

(L) A light-chain variable region that includes
a light-chain complementarity determining region (CDRL) 1, a CDRL2, and a CDRL3,
wherein the CDRL1 is a polypeptide that includes an amino acid sequence of (L1),
the CDRL2 is a polypeptide that includes an amino acid sequence of (L2),
the CDRL3 is a polypeptide that includes an amino acid sequence of (L3),
the amino acid sequences of (L1), (L2), and (L3) are as follows:
(L1) an amino acid sequence of (L1-1), (L1-2), or (L1-3) below:
   (L1-1) any one of amino acid sequences of CDRL1 shown in Conditions (L1) and (L2) below,
   (L1-2) an amino acid sequence having 80% or more identity to the amino acid sequence of (L1-1), and
   (L1-3) an amino acid sequence consisting of the amino acid sequence of (L1-1) with deletion, substitution, insertion, and/or addition of one or several amino acids;
(L2) an amino acid sequence of (L2-1), (L2-2), or (L2-3) below:
   (L2-1) any one of amino acid sequences of CDRL2 shown in Conditions (L1) and (L2) below,
   (L2-2) an amino acid sequence having 80% or more identity to the amino acid sequence of (L2-1), and
   (L2-3) an amino acid sequence consisting of the amino acid sequence of (L2-1) with deletion, substitution, insertion, and/or addition of one or several amino acids; and
(L3) an amino acid sequence of (L3-1), (L3-2), or (L3-3) below:
   (L3-1) any one of amino acid sequences of CDRL3 shown in Conditions (L1) and (L2) below,
   (L3-2) an amino acid sequence having 80% or more identity to the amino acid sequence of (L3-1), and
   (L3-3) an amino acid sequence consisting of the amino acid sequence of (L3-1) with deletion, substitution, insertion, and/or addition of one or several amino acids,

Condition (L1) is as follows:
the CDRL1 is QX₁X₂SX₃X₄ (Sequence ID No. 313),
the CDRL2 is AAS (Sequence ID No. 34) or GAS (Sequence ID No. 63),
the CDRL3 is QQYX₅X₆X₇X₈X₉X₁₀X₁₁ (Sequence ID No. 314),
X₁ is S or D,
X₂ is I or V,
X₃ is S or R,
X₄ is Y or N,
X₅ is Y, E, N, or D,
X₆ is S or N,
X₇ is not present or is T, Y, or S,
X₈ is P, S, R, or L,
X₉ is Q, R, P, or I,
X₁₀ is not present or is S or C, and
X₁₁ is not present or is T, and

Condition (L2) is as follows:
the CDRL1 is X₁X₂DVGX₃YX₄X₅ (Sequence ID No. 315),
the CDRL2 is DVN (Sequence ID No. 71), DVI (Sequence ID No. 75), or DVS (Sequence ID No. 79),
the CDRL3 is X₆SX₇X₈X₉X₁₀X₁₁X₁₂X₁₃X₁₄X₁₅V (Sequence ID No. 316),
X₁ is S or G,
X₂ is R or S,
X₃ is G, A, or S,
X₄ is N, D, E, or Q,
X₅ is Y, F, L, or W,
X₆ is W, S, or C,
X₇ is F, Y, or W,
X₈ is not present or is A or T,
X₉ is not present or is G,
X₁₀ is not present or is S,
X₁₁ is G or S,
X₁₂ is not present or is S,
X₁₃ is S, T, or R,
X₁₄ is Y, N, T, F, P, or R, and
X₁₅ is Y, S, W, A, or F.

Examples of heavy-chain variable regions that meet Condition (H1) above include the heavy-chain variable regions of (HA), (HB), and (HC) above.

Regarding the light-chain variable region of (L) above, (L1-1), (L2-1), and (L3-1) meet the same condition or different conditions of Conditions (L1) and (L2), and preferably meet the same condition.

In Condition (L1), it is preferable that X₇ is not present or is T or Y.

In Condition (L2), it is preferable that X₄ is N, D, or E. It is preferable that X₅ is Y, F, or L. It is preferable that X₇ is F or Y.

Examples of light-chain variable regions that meet Condition (L1) above include the light-chain variable regions of (LB) to (LJ) above.

Examples of light-chain variable regions that meet Condition (L2) above include the light-chain variable regions of (LA) and (LK) to (LQ) above.

Regarding the heavy-chain variable region of (H) above and the light-chain variable region of (L) above, if the CDRH1 is GFTFSTYQ (Sequence ID No. 21), the CDRH2 is IVSSGGST (Sequence ID No. 22), and the CDRH3 is AGELLPYYGMDV (Sequence ID No. 23) in the heavy-chain variable region of (H) above, it is preferable that the CDRL1 is SRDVGGYNY (Sequence ID No. 29), the CDRL2 is DVI (Sequence ID No. 30), and the CDRL3 is an amino acid sequence other than WSFAGSYYV (Sequence ID No. 31) in the light-chain variable region of (L) above.

### Second Antibody and Antigen-Binding Fragment Thereof

The antibody of the present invention against CD19 or the antigen-binding fragment thereof (also referred to as a “second antigen or the like” hereinafter) includes the heavy-chain variable region of (H) below and the light-chain variable region of (L) below. The antibody of the present invention or the like is characterized by including the heavy-chain variable region of (H) below and the light-chain variable region of (L) below, and there is no particular limitation on the other configurations and conditions. The antibody of the present invention or the like is capable of binding to CD19. It is known that CD19 is expressed in normal B cells and specific cancer cells derived from B-cell lymphoma and the like, for example. Accordingly, the antibody of the present invention or the like can be favorably used as a bispecific antibody against CD19-expressing cancer cells, the antigen-binding domain of a CAR in CAR-T cells, and the like, for example. The descriptions of the CAR library, the first screening method, the first antibody or antigen-binding fragment thereof, and the like of the present invention can be applied.

(H) A heavy-chain variable region that includes
a heavy-chain complementarity determining region (CDRH) 1, a CDRH2, and a CDRH3,
wherein the CDRH1 is a polypeptide that includes an amino acid sequence of (H1),
the CDRH2 is a polypeptide that includes an amino acid sequence of (H2),
the CDRH3 is a polypeptide that includes an amino acid sequence of (H3), and
the amino acid sequences of (H1), (H2), and (H3) are as follows:
(H1) an amino acid sequence of (H1-1), (H1-2), or (H1-3) below:
   (H1-1) any one of amino acid sequences of CDRH1 shown in Table 3A below,
   (H1-2) an amino acid sequence having 80% or more identity to the amino acid sequence of (H1-1), and
   (H1-3) an amino acid sequence consisting of the amino acid sequence of (H1-1) with deletion, substitution, insertion, and/or addition of one or several amino acids;
(H2) an amino acid sequence of (H2-1), (H2-2), or (H2-3) below:
   (H2-1) any one of amino acid sequences of CDRH2 shown in Table 3A below,
   (H2-2) an amino acid sequence having 80% or more identity to the amino acid sequence of (H2-1), and
   (H2-3) an amino acid sequence consisting of the amino acid sequence of (H2-1) with deletion, substitution, insertion, and/or addition of one or several amino acids; and
(H3) an amino acid sequence of (H3-1), (H3-2), or (H3-3) below:
   (H3-1) any one of amino acid sequences of CDRH3 shown in Table 3A below,
   (H3-2) an amino acid sequence having 80% or more identity to the amino acid sequence of (H3-1), and
   (H3-3) an amino acid sequence consisting of the amino acid sequence of (H3-1) with deletion, substitution, insertion, and/or addition of one or several amino acids.

(L) A light-chain variable region that includes
a light-chain complementarity determining region (CDRL) 1, a CDRL2, and a CDRL3,
wherein the CDRL1 is a polypeptide that includes an amino acid sequence of (L1),
the CDRL2 is a polypeptide that includes an amino acid sequence of (L2),
the CDRL3 is a polypeptide that includes an amino acid sequence of (L3), and
the amino acid sequences of (L1), (L2), and (L3) are as follows:
(L1) an amino acid sequence of (L1-1), (L1-2), or (L1-3) below:
   (L1-1) any one of amino acid sequences of CDRL1 shown in Table 3B below,
   (L1-2) an amino acid sequence having 80% or more identity to the amino acid sequence of (L1-1), and
   (L1-3) an amino acid sequence consisting of the amino acid sequence of (L1-1) with deletion, substitution, insertion, and/or addition of one or several amino acids;
(L2) an amino acid sequence of (L2-1), (L2-2), or (L2-3) below:
   (L2-1) any one of amino acid sequences of CDRL2 shown in Table 3B below,
   (L2-2) an amino acid sequence having 80% or more identity to the amino acid sequence of (L2-1), and
   (L2-3) an amino acid sequence consisting of the amino acid sequence of (L2-1) with deletion, substitution, insertion, and/or addition of one or several amino acids; and
(L3) an amino acid sequence of (L3-1), (L3-2), or (L3-3) below:
   (L3-1) any one of amino acid sequences of CDRL3 shown in Table 3B below,
   (L3-2) an amino acid sequence having 80% or more identity to the amino acid sequence of (L3-1), and
   (L3-3) an amino acid sequence consisting of the amino acid sequence of (L3-1) with deletion, substitution, insertion, and/or addition of one or several amino acids.

TABLE 3A

| Heavy-chain | | | | |
|---|---|---|---|---|
| variable region | CDRH1 | CDRH2 | CDRH3 | |
| (HA) | GFTFDDYA | ISWNSGRI | ARDQGYHYYDSAEHAFDI | 18H |
| | (Sequence ID No. 216) | (Sequence ID No. 217) | (Sequence ID No. 218) | |

TABLE 3B

| Light-chain | | | | |
|---|---|---|---|---|
| variable region | CDRL1 | CDRL2 | CDRL3 | |
| (LA) | KLGDKY | QDS | QAWDSSTHVV | L4 |
| | (Sequence ID No. 220) | (Sequence ID No. 221) | (Sequence ID No. 222) | |
| (LB) | SSDVGGYNY | DVS | GTWDTSLTAVV | L7 |
| | (Sequence ID No. 224) | (Sequence ID No. 225) | (Sequence ID No. 226) | |
| (LC) | WSNIGDDH | DTS | GTWESSLSGVV | L9 |
| | (Sequence ID No. 228) | (Sequence ID No. 229) | (Sequence ID No. 230) | |
| (LD) | SSDVGGYDY | DVT | SSYTTSTTWV | L13 |
| | (Sequence ID No. 232) | (Sequence ID No. 233) | (Sequence ID No. 234) | |
| (LE) | TSDVGTTNY | DVT | SYAGSYTFVV | L14 |
| | (Sequence ID No. 236) | (Sequence ID No. 237) | (Sequence ID No. 238) | |
| (LF) | SSDVGVYNY | DVS | AAWDDSLNGVV | L16 |
| | (Sequence ID No. 240) | (Sequence ID No. 241) | (Sequence ID No. 242) | |
| (LG) | SSNIGNNY | DNY | AAWDDSLSAI | L17 |
| | (Sequence ID No. 244) | (Sequence ID No. 245) | (Sequence ID No. 246) | |
| (LH) | SSDVGGYNY | DVS | HSYDSSLSHV | L22 |
| | (Sequence ID No. 248) | (Sequence ID No. 249) | (Sequence ID No. 250) | |
| (LI) | QSVSSN | GAS | QQYNNWPPLYT | K4 |
| | (Sequence ID No. 252) | (Sequence ID No. 253) | (Sequence ID No. 254) | |
| (LJ) | QSVSSY | DAS | QQSYSTLLYT | K5 |
| | (Sequence ID No. 256) | (Sequence ID No. 257) | (Sequence ID No. 258) | |
| (LK) | QSVSSY | DAS | QQYDSLPLT | K6 |
| | (Sequence ID No. 260) | (Sequence ID No. 261) | (Sequence ID No. 262) | |
| (LL) | QTISASS | GAS | QQFNEWPLT | K9 |
| | (Sequence ID No. 264) | (Sequence ID No. 265) | (Sequence ID No. 266) | |
| (LM) | QSVSSN | GAS | QQYGSSPDIFT | K10 |
| | (Sequence ID No. 268) | (Sequence ID No. 269) | (Sequence ID No. 270) | |

In the present invention, “CD19” is a type-I transmembrane glycoprotein having a molecular weight of 95 kDa, for example. It is known that CD19 is involved in the generation, activation, differentiation regulation, and the like of B cells, for example, and CD19 is mainly expressed in B cells. Human CD19 has an amino acid sequence that corresponds to the amino acid sequence registered as NCBI Accession No. NM_001770.6, for example. Mouse CD19 has an amino acid sequence that corresponds to the amino acid sequence registered as NCBI Accession No. NM_009844.2, for example.

In the heavy-chain variable region of (H) above, the CDRH1 is the CDRH1 of (HA) above. The CDRH2 is the CDRH2 of (HA) above. The CDRH3 is the CDRH3 of (HA) above.

In the light-chain variable region of (L) above, the CDRL1 is the CDRL1 of any one of (LA) to (LL) and (LM) above. The CDRL2 is the CDRL2 of any one of (LA) to (LL) and (LM) above. The CDRL3 is the CDRL3 of any one of (LA) to (LL) and (LM) above.

The CDRL1, the CDRL2, and the CDRL3 may belong to the same one or different ones of (LA) to (LL) and (LM) above, and they preferably belong to the same one. If the CDRL1, the CDRL2, and the CDRL3 belong to the same one of (LA) to (LL) and (LM) above, the CDRL1, the CDRL2, and the CDRL3 are those of any one of (LA) to (LL) and (LM) above.

The term “identity” as used for the CDRs refers to the degree of identity when appropriately aligning sequences to be compared, for example, and means the ratio (%) of exactly the same amino acids in these sequences. The “identity” in each case refers to 80% or more, 85% or more, 90% or more, 95% or more, 96% or more, 97% or more, 98% or more, or 99% or more identity, for example. The identity can be calculated with default parameters using analysis software such as BLAST or FASTA (the same applies hereinafter).

The term “one or several” as used for the CDRs regarding substitution and the like refers to 1 to 5, 1 to 4, 1 to 3, 1 or 2, or 1, for example.

The amino acid substitution may be conservative substitution, for example (the same applies hereinafter). The term “conservative substitution” means that one or several amino acids are substituted by other amino acids and/or amino acid derivatives such that the functions of a protein are not substantially modified. It is preferable that a “substituting amino acid” and an “amino acid to be substituted” have similar properties and/or functions, for example. Specifically, it is preferable that they are similar in chemical properties such as a hydrophobicity/hydrophilicity index (hydropathy), a polarity, and an electric charge, physical properties such as a secondary structure, and the like, for example. Amino acids or amino acid derivatives having similar properties and/or functions are known in the art, for example. Specific examples of nonpolar amino acids (hydrophobic amino acids) include alanine, valine, isoleucine, leucine, proline, tryptophan, phenylalanine, and methionine; specific examples of polar amino acids (neutral amino acids) include glycine, serine, threonine, tyrosine, glutamine, asparagine, and cysteine; specific examples of positively charged amino acids (basic amino acids) include arginine, histidine, and lysine; and specific examples of negatively charged amino acids (acidic amino acids) include aspartic acid and glutamic acid.

In the light-chain variable region of (L) above, there is no particular limitation on the combination of (L1-1), (L2-1), and (L3-1) above, and the CDRL1 of any one of (LA) to (LL) and (LM) above, the CDRL2 of any one of (LA) to (LL) and (LM) above, and the CDRL3 of any one of (LA) to (LL) and (LM) above can be combined as desired, for example. The combination of (L1-1), (L2-1), and (L3-1) above is preferably a combination of the CDRL1, the CDRL2, and the CDRL3 of any one of (LA) to (LL) and (LM) above.

Hereinafter, the combination of the heavy-chain variable region or heavy chain and the light-chain variable region or light chain in the antibody of the present invention or the like will be described more specifically. In this combination, the descriptions of the heavy-chain variable region can be applied to the heavy chain, and vice versa. Also, in this combination, the descriptions of the light-chain variable region can be applied to the light chain, and vice versa. In the amino acid sequences and the base sequences shown below, underlined amino acid sequences and base sequences are amino acid sequences that correspond to the CDRs and base sequences coding for the amino acid sequences that correspond to the CDRs, respectively, unless otherwise stated.

As described above, the combination of the heavy-chain variable region and the light-chain variable region in the antibody of the present invention or the like is a combination of (HA) above and any one of (LA) to (LL) and (LM) above, for example.

### Combination of (HA) and (LA) (Combination (LA))

The antibodies or the like of Combination (LA) are also referred to as an antibody 18H-L4 group, for example. In Combination (LA) above, the heavy-chain variable region of (HA) above includes the CDRH1, the CDRH2, and the CDRH3, the CDRH1 is a polypeptide that includes the amino acid sequence of (H1-A) below, the CDRH2 is a polypeptide that includes the amino acid sequence of (H2-A) below, and the CDRH3 is a polypeptide that includes the amino acid sequence of (H3-A) below. The light-chain variable region of (LA) above includes the CDRL1, the CDRL2, and the CDRL3, the CDRL1 is a polypeptide that includes the amino acid sequence of (L1-A) below, the CDRL2 is a polypeptide that includes the amino acid sequence of (L2-A) below, and the CDRL3 is a polypeptide that includes the amino acid sequence of (L3-A) below.

(H1-A) An amino acid sequence of (H1-A1), (H1-A2), or (H1-A3) below:
(H1-A1) an amino acid sequence of Sequence ID No. 216 (GFTEDDYA),
(H1-A2) an amino acid sequence having 80% or more identity to the amino acid sequence of Sequence ID No. 216, and
(H1-A3) an amino acid sequence consisting of the amino acid sequence of Sequence ID No. 216 with deletion, substitution, insertion, and/or addition of one or several amino acids.

(H2-A) An amino acid sequence of (H2-A1), (H2-A2), or (H2-A3) below:
(H2-A1) an amino acid sequence of Sequence ID No. 217 (ISWNSGRI),
(H2-A2) an amino acid sequence having 80% or more identity to the amino acid sequence of Sequence ID No. 217, and
(H2-A3) an amino acid sequence consisting of the amino acid sequence of Sequence ID No. 217 with deletion, substitution, insertion, and/or addition of one or several amino acids.

(H3-A) An amino acid sequence of (H3-A1), (H3-A2), or (H3-A3) below:
(H3-A1) an amino acid sequence of Sequence ID No. 218 (ARDQGYHYYDSAEHAFDI),
(H3-A2) an amino acid sequence having 80% or more identity to the amino acid sequence of Sequence ID No. 218, and
(H3-A3) an amino acid sequence consisting of the amino acid sequence of Sequence ID No. 218 with deletion, substitution, insertion, and/or addition of one or several amino acids.

(L1-A) An amino acid sequence of (L1-A1), (L1-A2), or (L1-A3) below:
(L1-A1) an amino acid sequence of Sequence ID No. 220 (KLGDKY),
(L1-A2) an amino acid sequence having 80% or more identity to the amino acid sequence of Sequence ID No. 220, and
(L1-A3) an amino acid sequence consisting of the amino acid sequence of Sequence ID No. 220 with deletion, substitution, insertion, and/or addition of one or several amino acids.

(L2-A) An amino acid sequence of (L2-A1), (L2-A2), or (L2-A3) below:
(L2-A1) an amino acid sequence of Sequence ID No. 221 (QDS),
(L2-A2) an amino acid sequence having 80% or more identity to the amino acid sequence of Sequence ID No. 221, and
(L2-A3) an amino acid sequence consisting of the amino acid sequence of Sequence ID No. 221 with deletion, substitution, insertion, and/or addition of one or several amino acids.

(L3-A) An amino acid sequence of (L3-A1), (L3-A2), or (L3-A3) below:
(L3-A1) an amino acid sequence of Sequence ID No. 222 (QAWDSSTHVV),
(L3-A2) an amino acid sequence having 80% or more identity to the amino acid sequence of Sequence ID No. 222, and
(L3-A3) an amino acid sequence consisting of the amino acid sequence of Sequence ID No. 222 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LA) above, the heavy-chain variable region of (HA) above includes a polypeptide consisting of the amino acid sequence of (H-A) below, for example. The light-chain variable region of (LA) above includes a polypeptide consisting of the amino acid sequence of (L-A) below, for example.

(H-A) An amino acid sequence of (H-A1), (H-A2), or (H-A3) below:
(H-A1) an amino acid sequence of Sequence ID No. 219:

Sequence ID No. 219:
EVQLVESGGGLVQPGRSLRLSCAASGFTFDDYAMHWVRQAPGKGLEWVS
GISWNSGRIGYADSVKGRFTISRDNAKNSLFLQMNSLRAEDTAVYYCAR
DQGYHYYDSAEHAFDIWGQGTVVTVSS,

(H-A2) an amino acid sequence having 80% or more identity to the amino acid sequence of Sequence ID No. 219, and
(H-A3) an amino acid sequence consisting of the amino acid sequence of Sequence ID No. 219 with deletion, substitution, insertion, and/or addition of one or several amino acids.

(L-A) An amino acid sequence of (L-A1), (L-A2), or (L-A3) below:
(L-A1) an amino acid sequence of Sequence ID No. 223:

Sequence ID No. 223:
SYELTQPPSVSVSPGQTASITCSGDKLGDKYACWYQQKPGQSPVLVIYQ
DSKRPSGIPERFSGSNSGNTATLTISGTQAMDEADYYCQAWDSSTHVVF
GGGTKLTVL,

(L-A2) an amino acid sequence having 80% or more identity to the amino acid sequence of Sequence ID No. 223, and
(L-A3) an amino acid sequence consisting of the amino acid sequence of Sequence ID No. 223 with deletion, substitution, insertion, and/or addition of one or several amino acids.

The amino acid sequence of (H-A1) above is a sequence that includes the amino acid sequences of (H1-A1) of the CDRH1, (H2-A1) of the CDRH2, and (H3-A1) of the CDRH3, for example. The amino acid sequence of (H-A2) above may be an amino acid sequence that includes the amino acid sequences of (H1-A1) of the CDRH1, (H2-A1) of the CDRH2, and (H3-A1) of the CDRH3 and that has 80% or more identity to the amino acid sequence of Sequence ID No. 219, for example. The amino acid sequence of (H-A3) above may be an amino acid sequence that includes the amino acid sequences of (H1-A1) of the CDRH1, (H2-A1) of the CDRH2, and (H3-A1) of the CDRH3 and that consists of the amino acid sequence of Sequence ID No. 219 with deletion, substitution, insertion, and/or addition of one or several amino acids, for example.

The amino acid sequence of (L-A1) above is a sequence that includes the amino acid sequences of (L1-A1) of the CDRL1, (L2-A1) of the CDRL2, and (L3-A1) of the CDRL3, for example. The amino acid sequence of (L-A2) above may be an amino acid sequence that includes the amino acid sequences of (L1-A1) of the CDRL1, (L2-A1) of the CDRL2, and (L3-A1) of the CDRL3 and that has 80% or more identity to the amino acid sequence of Sequence ID No. 223, for example. The amino acid sequence of (L-A3) above may be an amino acid sequence that includes the amino acid sequences of (L1-A1) of the CDRL1, (L2-A1) of the CDRL2, and (L3-A1) of the CDRL3 and that consists of the amino acid sequence of Sequence ID No. 223 with deletion, substitution, insertion, and/or addition of one or several amino acids, for example.

In the antibody of the present invention, the heavy-chain variable region is (H-A1) above, and the light-chain variable region is (L-A1) above, for example. The antibody that includes this combination is also referred to as an “antibody 18H-L4” hereinafter.

The “identity” as used for the polypeptide of the heavy-chain variable region and the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the heavy-chain variable region and the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LB) (Combination (LB))

The antibodies or the like of Combination (LB) are also referred to as an antibody 18H-L7 group, for example. In Combination (LB) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LB) above includes the CDRL1, the CDRL2, and the CDRL3, the CDRL1 is a polypeptide that includes the amino acid sequence of (L1-B) below, the CDRL2 is a polypeptide that includes the amino acid sequence of (L2-B) below, and the CDRL3 is a polypeptide that includes the amino acid sequence of (L3-B) below.

(L1-B) An amino acid sequence of (L1-B1), (L1-B2), or (L1-B3) below:
(L1-B1) an amino acid sequence of Sequence ID No. 224 (SSDVGGYNY),
(L1-B2) an amino acid sequence having 80% or more identity to the amino acid sequence of Sequence ID No. 224, and
(L1-B3) an amino acid sequence consisting of the amino acid sequence of Sequence ID No. 224 with deletion, substitution, insertion, and/or addition of one or several amino acids.

(L2-B) An amino acid sequence of (L2-B1), (L2-B2), or (L2-B3) below:
(L2-B1) an amino acid sequence of Sequence ID No. 225 (DVS),
(L2-B2) an amino acid sequence having 80% or more identity to the amino acid sequence of Sequence ID No. 225, and
(L2-B3) an amino acid sequence consisting of the amino acid sequence of Sequence ID No. 225 with deletion, substitution, insertion, and/or addition of one or several amino acids.

(L3-B) An amino acid sequence of (L3-B1), (L3-B2), or (L3-B3) below:
(L3-B1) an amino acid sequence of Sequence ID No. 226 (GTWDTSLTAVV),
(L3-B2) an amino acid sequence having 80% or more identity to the amino acid sequence of Sequence ID No. 226, and
(L3-B3) an amino acid sequence consisting of the amino acid sequence of Sequence ID No. 226 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LB) above, the heavy-chain variable region of (HA) above includes a polypeptide consisting of the amino acid sequence of (H-A) above, for example. The light-chain variable region of (LB) above includes a polypeptide consisting of the amino acid sequence of (L-B) below, for example.

(L-B) An amino acid sequence of (L-B1), (L-B2), or (L-B3) below:
(L-B1) an amino acid sequence of Sequence ID No. 227:

Sequence ID No. 227:
QSALTQPRSVSGSPGQSVTISCTGTSSDVGGYNYVSWYQQHPGKAPKLM
IYDVSNRPSGVSNRFSGSKSGNTASLTISGLQTGDEADYYCGTWDTSLT
AVVFGGGTELTVL,

(L-B2) an amino acid sequence having 80% or more identity to the amino acid sequence of Sequence ID No. 227, and
(L-B3) an amino acid sequence consisting of the amino acid sequence of Sequence ID No. 227 with deletion, substitution, insertion, and/or addition of one or several amino acids.

The amino acid sequence of (L-B1) above is a sequence that includes the amino acid sequences of (L1-B1) of the CDRL1, (L2-B1) of the CDRL2, and (L3-B1) of the CDRL3, for example. The amino acid sequence of (L-B2) above may be an amino acid sequence that includes the amino acid sequences of (L1-B1) of the CDRL1, (L2-B1) of the CDRL2, and (L3-B1) of the CDRL3 and that has 80% or more identity to the amino acid sequence of Sequence ID No. 227, for example. The amino acid sequence of (L-B3) above may be an amino acid sequence that includes the amino acid sequences of (L1-B1) of the CDRL1, (L2-B1) of the CDRL2, and (L3-B1) of the CDRL3 and that consists of the amino acid sequence of Sequence ID No. 227 with deletion, substitution, insertion, and/or addition of one or several amino acids, for example.

In the antibody of the present invention, the heavy-chain variable region is (H-A1) above, and the light-chain variable region is (L-B1) above, for example. The antibody that includes this combination is also referred to as an “antibody 18H-L7” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LC) (Combination (LC))

The antibodies or the like of Combination (LC) are also referred to as an antibody 18H-L9 group, for example. In Combination (LC) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LC) above includes the CDRL1, the CDRL2, and the CDRL3, the CDRL1 is a polypeptide that includes the amino acid sequence of (L1-C) below, the CDRL2 is a polypeptide that includes the amino acid sequence of (L2-C) below, and the CDRL3 is a polypeptide that includes the amino acid sequence of (L3-C) below.

(L1-C) An amino acid sequence of (L1-C1), (L1-C2), or (L1-C3) below:
(L1-C1) an amino acid sequence of Sequence ID No. 228 (WSNIGDDH),
(L1-C2) an amino acid sequence having 80% or more identity to the amino acid sequence of Sequence ID No. 228, and
(L1-C3) an amino acid sequence consisting of the amino acid sequence of Sequence ID No. 228 with deletion, substitution, insertion, and/or addition of one or several amino acids.

(L2-C) An amino acid sequence of (L2-C1), (L2-C2), or (L2-C3) below:
(L2-C1) an amino acid sequence of Sequence ID No. 229 (DTS),
(L2-C2) an amino acid sequence having 80% or more identity to the amino acid sequence of Sequence ID No. 229, and
(L2-C3) an amino acid sequence consisting of the amino acid sequence of Sequence ID No. 229 with deletion, substitution, insertion, and/or addition of one or several amino acids.

(L3-C) An amino acid sequence of (L3-C1), (L3-C2), or (L3-C3) below:
(L3-C1) an amino acid sequence of Sequence ID No. 230 (GTWESSLSGVV),
(L3-C2) an amino acid sequence having 80% or more identity to the amino acid sequence of Sequence ID No. 230, and
(L3-C3) an amino acid sequence consisting of the amino acid sequence of Sequence ID No. 230 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LC) above, the heavy-chain variable region of (HA) above includes a polypeptide consisting of the amino acid sequence of (H-A) above, for example. The light-chain variable region of (LC) above includes a polypeptide consisting of the amino acid sequence of (L-C) below, for example.

(L-C) An amino acid sequence of (L-C1), (L-C2), or (L-C3) below:
(L-C1) an amino acid sequence of Sequence ID No. 231:

Sequence ID No. 231:
QSVLTQPPSVSAAPGQKVTISCSGSWSNIGDDHVSWYQQFPGAAPKLLI
YDTSKRPSRVADRFSGSKSGASATLAITGLQAGDEADYYCGTWESSLSG
VVFGGGTELTVL,

(L-C2) an amino acid sequence having 80% or more identity to the amino acid sequence of Sequence ID No. 231, and
(L-C3) an amino acid sequence consisting of the amino acid sequence of Sequence ID No. 231 with deletion, substitution, insertion, and/or addition of one or several amino acids.

The amino acid sequence of (L-C1) above is a sequence that includes the amino acid sequences of (L1-C1) of the CDRL1, (L2-C1) of the CDRL2, and (L3-C1) of the CDRL3, for example. The amino acid sequence of (L-C2) above may be an amino acid sequence that includes the amino acid sequences of (L1-C1) of the CDRL1, (L2-C1) of the CDRL2, and (L3-C1) of the CDRL3 and that has 80% or more identity to the amino acid sequence of Sequence ID No. 231, for example. The amino acid sequence of (L-C3) above may be an amino acid sequence that includes the amino acid sequences of (L1-C1) of the CDRL1, (L2-C1) of the CDRL2, and (L3-C1) of the CDRL3 and that consists of the amino acid sequence of Sequence ID No. 231 with deletion, substitution, insertion, and/or addition of one or several amino acids, for example.

In the antibody of the present invention, the heavy-chain variable region is (H-A1) above, and the light-chain variable region is (L-C1) above, for example. The antibody that includes this combination is also referred to as an “antibody 18H-L9” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LD) (Combination (LD))

The antibodies or the like of Combination (LD) are also referred to as an antibody 18H-L13 group, for example. In Combination (LD) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LD) above includes the CDRL1, the CDRL2, and the CDRL3, the CDRL1 is a polypeptide that includes the amino acid sequence of (L1-D) below, the CDRL2 is a polypeptide that includes the amino acid sequence of (L2-D) below, and the CDRL3 is a polypeptide that includes the amino acid sequence of (L3-D) below.

(L1-D) An amino acid sequence of (L1-D1), (L1-D2), or (L1-D3) below:
(L1-D1) an amino acid sequence of Sequence ID No. 232 (SSDVGGYDY),
(L1-D2) an amino acid sequence having 80% or more identity to the amino acid sequence of Sequence ID No. 232, and
(L1-D3) an amino acid sequence consisting of the amino acid sequence of Sequence ID No. 232 with deletion, substitution, insertion, and/or addition of one or several amino acids.

(L2-D) An amino acid sequence of (L2-D1), (L2-D2), or (L2-D3) below:
(L2-D1) an amino acid sequence of Sequence ID No. 233 (DVT),
(L2-D2) an amino acid sequence having 80% or more identity to the amino acid sequence of Sequence ID No. 233, and
(L2-D3) an amino acid sequence consisting of the amino acid sequence of Sequence ID No. 233 with deletion, substitution, insertion, and/or addition of one or several amino acids.

(L3-D) An amino acid sequence of (L3-D1), (L3-D2), or (L3-D3) below:
(L3-D1) an amino acid sequence of Sequence ID No. 234 (SSYTTSTTWV),
(L3-D2) an amino acid sequence having 80% or more identity to the amino acid sequence of Sequence ID No. 234, and
(L3-D3) an amino acid sequence consisting of the amino acid sequence of Sequence ID No. 234 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LD) above, the heavy-chain variable region of (HA) above includes a polypeptide consisting of the amino acid sequence of (H-A) above, for example. The light-chain variable region of (LD) above includes a polypeptide consisting of the amino acid sequence of (L-D) below, for example.

(L-D) An amino acid sequence of (L-D1), (L-D2), or (L-D3) below:
(L-D1) an amino acid sequence of Sequence ID No. 235:

Sequence ID No. 235:
QSALTQPRSVSGSPGQSVTISCTGTSSDVGGYNYVSWYQQHPGKA
PKLMIYDVSNRPSGVSNRFSGSKSGNTASLTISGLQTGDEADYYC
GTWDTSLTAVVFGGGTELTVL,

(L-D2) an amino acid sequence having 80% or more identity to the amino acid sequence of Sequence ID No. 235, and
(L-D3) an amino acid sequence consisting of the amino acid sequence of Sequence ID No. 235 with deletion, substitution, insertion, and/or addition of one or several amino acids.

The amino acid sequence of (L-D1) above is a sequence that includes the amino acid sequences of (L1-D1) of the CDRL1, (L2-D1) of the CDRL2, and (L3-D1) of the CDRL3, for example. The amino acid sequence of (L-D2) above may be an amino acid sequence that includes the amino acid sequences of (L1-D1) of the CDRL1, (L2-D1) of the CDRL2, and (L3-D1) of the CDRL3 and that has 80% or more identity to the amino acid sequence of Sequence ID No. 235, for example. The amino acid sequence of (L-D3) above may be an amino acid sequence that includes the amino acid sequences of (L1-D1) of the CDRL1, (L2-D1) of the CDRL2, and (L3-D1) of the CDRL3 and that consists of the amino acid sequence of Sequence ID No. 235 with deletion, substitution, insertion, and/or addition of one or several amino acids, for example.

In the antibody of the present invention, the heavy-chain variable region is (H-A1) above, and the light-chain variable region is (L-D1) above, for example. The antibody that includes this combination is also referred to as an “antibody 18H-L13” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LE) (Combination (LE))

The antibodies or the like of Combination (LE) are also referred to as an antibody 18H-L14 group, for example. In Combination (LE) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LE) above includes the CDRL1, the CDRL2, and the CDRL3, the CDRL1 is a polypeptide that includes the amino acid sequence of (L1-E) below, the CDRL2 is a polypeptide that includes the amino acid sequence of (L2-E) below, and the CDRL3 is a polypeptide that includes the amino acid sequence of (L3-E) below.

(L1-E) An amino acid sequence of (L1-E1), (L1-E2), or (L1-E3) below:
(L1-E1) an amino acid sequence of Sequence ID No. 236 (TSDVGTTNY),
(L1-E2) an amino acid sequence having 80% or more identity to the amino acid sequence of Sequence ID No. 236, and
(L1-E3) an amino acid sequence consisting of the amino acid sequence of Sequence ID No. 236 with deletion, substitution, insertion, and/or addition of one or several amino acids.

(L2-E) An amino acid sequence of (L2-E1), (L2-E2), or (L2-E3) below:
(L2-E1) an amino acid sequence of Sequence ID No. 237 (DVT),
(L2-E2) an amino acid sequence having 80% or more identity to the amino acid sequence of Sequence ID No. 237, and
(L2-E3) an amino acid sequence consisting of the amino acid sequence of Sequence ID No. 237 with deletion, substitution, insertion, and/or addition of one or several amino acids.

(L3-E) An amino acid sequence of (L3-E1), (L3-E2), or (L3-E3) below:
(L3-E1) an amino acid sequence of Sequence ID No. 238 (SYAGSYTFVV),
(L3-E2) an amino acid sequence having 80% or more identity to the amino acid sequence of Sequence ID No. 238, and
(L3-E3) an amino acid sequence consisting of the amino acid sequence of Sequence ID No. 238 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LE) above, the heavy-chain variable region of (HA) above includes a polypeptide consisting of the amino acid sequence of (H-A) above, for example. The light-chain variable region of (LE) above includes a polypeptide consisting of the amino acid sequence of (L-E) below, for example.

(L-E) An amino acid sequence of (L-E1), (L-E2), or (L-E3) below:
(L-E1) an amino acid sequence of Sequence ID No. 239:

Sequence ID No. 239:
QSALTQPASVSGSPGQSITISCTGTTSDVGTTNYVSWYQQHPGKA
PKLLIYDVTNRPSGVPDRFSGSKSANTASLTISGLQAEDEADYYC
CSYAGSYTFVVFGGGTELTVL,

(L-E2) an amino acid sequence having 80% or more identity to the amino acid sequence of Sequence ID No. 239, and
(L-E3) an amino acid sequence consisting of the amino acid sequence of Sequence ID No. 239 with deletion, substitution, insertion, and/or addition of one or several amino acids.

The amino acid sequence of (L-E1) above is a sequence that includes the amino acid sequences of (L1-E1) of the CDRL1, (L2-E1) of the CDRL2, and (L3-E1) of the CDRL3, for example. The amino acid sequence of (L-E2) above may be an amino acid sequence that includes the amino acid sequences of (L1-E1) of the CDRL1, (L2-E1) of the CDRL2, and (L3-E1) of the CDRL3 and that has 80% or more identity to the amino acid sequence of Sequence ID No. 239, for example. The amino acid sequence of (L-E3) above may be an amino acid sequence that includes the amino acid sequences of (L1-E1) of the CDRL1, (L2-E1) of the CDRL2, and (L3-E1) of the CDRL3 and that consists of the amino acid sequence of Sequence ID No. 239 with deletion, substitution, insertion, and/or addition of one or several amino acids, for example.

In the antibody of the present invention, the heavy-chain variable region is (H-A1) above, and the light-chain variable region is (L-E1) above, for example. The antibody that includes this combination is also referred to as an “antibody 18H-L14” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LF) (Combination (LF))

The antibodies or the like of Combination (LF) are also referred to as an antibody 18H-L16 group, for example. In Combination (LF) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LF) above includes the CDRL1, the CDRL2, and the CDRL3, the CDRL1 is a polypeptide that includes the amino acid sequence of (L1-F) below, the CDRL2 is a polypeptide that includes the amino acid sequence of (L2-F) below, and the CDRL3 is a polypeptide that includes the amino acid sequence of (L3-F) below.

(L1-F) An amino acid sequence of (L1-F1), (L1-F2), or (L1-F3) below:
(L1-F1) an amino acid sequence of Sequence ID No. 240 (SSDVGVYNY),
(L1-F2) an amino acid sequence having 80% or more identity to the amino acid sequence of Sequence ID No. 240, and
(L1-F3) an amino acid sequence consisting of the amino acid sequence of Sequence ID No. 240 with deletion, substitution, insertion, and/or addition of one or several amino acids.

(L2-F) An amino acid sequence of (L2-F1), (L2-F2), or (L2-F3) below:
(L2-F1) an amino acid sequence of Sequence ID No. 241 (DVS),
(L2-F2) an amino acid sequence having 80% or more identity to the amino acid sequence of Sequence ID No. 241, and
(L2-F3) an amino acid sequence consisting of the amino acid sequence of Sequence ID No. 241 with deletion, substitution, insertion, and/or addition of one or several amino acids.

(L3-F) An amino acid sequence of (L3-F1), (L3-F2), or (L3-F3) below:
(L3-F1) an amino acid sequence of Sequence ID No. 242 (AAWDDSLNGVV),
(L3-F2) an amino acid sequence having 80% or more identity to the amino acid sequence of Sequence ID No. 242, and
(L3-F3) an amino acid sequence consisting of the amino acid sequence of Sequence ID No. 242 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LF) above, the heavy-chain variable region of (HA) above includes a polypeptide consisting of the amino acid sequence of (H-A) above, for example. The light-chain variable region of (LF) above includes a polypeptide consisting of the amino acid sequence of (L-F) below, for example.

(L-F) An amino acid sequence of (L-F1), (L-F2), or (L-F3) below:
(L-F1) an amino acid sequence of Sequence ID No. 243:

Sequence ID No. 243:
QSALTQPPSASGSPGQSVTISCTGTSSDVGVYNYVSWYQQHPGKA
PKLMIYDVSKRPSGVPDRFSGSKSANTASLTISGLQAEDEADYYC
AAWDDSLNGVVFGGGTQLTVL,

(L-F2) an amino acid sequence having 80% or more identity to the amino acid sequence of Sequence ID No. 243, and
(L-F3) an amino acid sequence consisting of the amino acid sequence of Sequence ID No. 243 with deletion, substitution, insertion, and/or addition of one or several amino acids.

The amino acid sequence of (L-F1) above is a sequence that includes the amino acid sequences of (L1-F1) of the CDRL1, (L2-F1) of the CDRL2, and (L3-F1) of the CDRL3, for example. The amino acid sequence of (L-F2) above may be an amino acid sequence that includes the amino acid sequences of (L1-F1) of the CDRL1, (L2-F1) of the CDRL2, and (L3-F1) of the CDRL3 and that has 80% or more identity to the amino acid sequence of Sequence ID No. 243, for example. The amino acid sequence of (L-F3) above may be an amino acid sequence that includes the amino acid sequences of (L1-F1) of the CDRL1, (L2-F1) of the CDRL2, and (L3-F1) of the CDRL3 and that consists of the amino acid sequence of Sequence ID No. 243 with deletion, substitution, insertion, and/or addition of one or several amino acids, for example.

In the antibody of the present invention, the heavy-chain variable region is (H-A1) above, and the light-chain variable region is (L-F1) above, for example. The antibody that includes this combination is also referred to as an “antibody 18H-L16” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LG) (Combination (LG))

The antibodies or the like of Combination (LG) are also referred to as an antibody 18H-L17 group, for example. In Combination (LG) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LG) above includes the CDRL1, the CDRL2, and the CDRL3, the CDRL1 is a polypeptide that includes the amino acid sequence of (L1-G) below, the CDRL2 is a polypeptide that includes the amino acid sequence of (L2-G) below, and the CDRL3 is a polypeptide that includes the amino acid sequence of (L3-G) below.

(L1-G) An amino acid sequence of (L1-G1), (L1-G2), or (L1-G3) below:
(L1-G1) an amino acid sequence of Sequence ID No. 244 (SSNIGNNY),
(L1-G2) an amino acid sequence having 80% or more identity to the amino acid sequence of Sequence ID No. 244, and
(L1-G3) an amino acid sequence consisting of the amino acid sequence of Sequence ID No. 244 with deletion, substitution, insertion, and/or addition of one or several amino acids.

(L2-G) An amino acid sequence of (L2-G1), (L2-G2), or (L2-G3) below:
(L2-G1) an amino acid sequence of Sequence ID No. 245 (DNV),
(L2-G2) an amino acid sequence having 80% or more identity to the amino acid sequence of Sequence ID No. 245, and
(L2-G3) an amino acid sequence consisting of the amino acid sequence of Sequence ID No. 245 with deletion, substitution, insertion, and/or addition of one or several amino acids.

(L3-G) An amino acid sequence of (L3-G1), (L3-G2), or (L3-G3) below:
(L3-G1) an amino acid sequence of Sequence ID No. 246 (AAWDDSLSAI),
(L3-G2) an amino acid sequence having 80% or more identity to the amino acid sequence of Sequence ID No. 246, and
(L3-G3) an amino acid sequence consisting of the amino acid sequence of Sequence ID No. 246 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LG) above, the heavy-chain variable region of (HA) above includes a polypeptide consisting of the amino acid sequence of (H-A) above, for example. The light-chain variable region of (LG) above includes a polypeptide consisting of the amino acid sequence of (L-G) below, for example.

(L-G) An amino acid sequence of (L-G1), (L-G2), or (L-G3) below:
(L-G1) an amino acid sequence of Sequence ID No. 247:

Sequence ID No. 247:
QSVLTQPPSASGTPGQRVTISCSGSSSNIGNNYVCWYQHLPGTAP
KLLIYDNVKRPSGIPDRFSGSKSGTSASLAISGLRSEDEADYYCA
AWDDSLSAIFGGGTELTVL,

(L-G2) an amino acid sequence having 80% or more identity to the amino acid sequence of Sequence ID No. 247, and
(L-G3) an amino acid sequence consisting of the amino acid sequence of Sequence ID No. 247 with deletion, substitution, insertion, and/or addition of one or several amino acids.

The amino acid sequence of (L-G1) above is a sequence that includes the amino acid sequences of (L1-G1) of the CDRL1, (L2-G1) of the CDRL2, and (L3-G1) of the CDRL3, for example. The amino acid sequence of (L-G2) above may be an amino acid sequence that includes the amino acid sequences of (L1-G1) of the CDRL1, (L2-G1) of the CDRL2, and (L3-G1) of the CDRL3 and that has 80% or more identity to the amino acid sequence of Sequence ID No. 247, for example. The amino acid sequence of (L-G3) above may be an amino acid sequence that includes the amino acid sequences of (L1-G1) of the CDRL1, (L2-G1) of the CDRL2, and (L3-G1) of the CDRL3 and that consists of the amino acid sequence of Sequence ID No. 247 with deletion, substitution, insertion, and/or addition of one or several amino acids, for example.

In the antibody of the present invention, the heavy-chain variable region is (H-A1) above, and the light-chain variable region is (L-G1) above, for example. The antibody that includes this combination is also referred to as an “antibody 18H-L17” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LH) (Combination (LH))

The antibodies or the like of Combination (LH) are also referred to as an antibody 18H-L22 group, for example. In Combination (LH) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LH) above includes the CDRL1, the CDRL2, and the CDRL3, the CDRL1 is a polypeptide that includes the amino acid sequence of (L1-H) below, the CDRL2 is a polypeptide that includes the amino acid sequence of (L2-H) below, and the CDRL3 is a polypeptide that includes the amino acid sequence of (L3-H) below.

(L1-H) An amino acid sequence of (L1-H1), (L1-H2), or (L1-H3) below:
(L1-H1) an amino acid sequence of Sequence ID No. 248 (SSDVGGYNY),
(L1-H2) an amino acid sequence having 80% or more identity to the amino acid sequence of Sequence ID No. 248, and
(L1-H3) an amino acid sequence consisting of the amino acid sequence of Sequence ID No. 248 with deletion, substitution, insertion, and/or addition of one or several amino acids.

(L2-H) An amino acid sequence of (L2-H1), (L2-H2), or (L2-H3) below:
(L2-H1) an amino acid sequence of Sequence ID No. 249 (DVS),
(L2-H2) an amino acid sequence having 80% or more identity to the amino acid sequence of Sequence ID No. 249, and
(L2-H3) an amino acid sequence consisting of the amino acid sequence of Sequence ID No. 249 with deletion, substitution, insertion, and/or addition of one or several amino acids.

(L3-H) An amino acid sequence of (L3-H1), (L3-H2), or (L3-H3) below:
(L3-H1) an amino acid sequence of Sequence ID No. 250 (HSYDSSLSHV),
(L3-H2) an amino acid sequence having 80% or more identity to the amino acid sequence of Sequence ID No. 250, and
(L3-H3) an amino acid sequence consisting of the amino acid sequence of Sequence ID No. 250 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LH) above, the heavy-chain variable region of (HA) above includes a polypeptide consisting of the amino acid sequence of (H-A) above, for example. The light-chain variable region of (LH) above includes a polypeptide consisting of the amino acid sequence of (L-H) below, for example.

(L-H) An amino acid sequence of (L-H1), (L-H2), or (L-H3) below:
(L-H1) an amino acid sequence of Sequence ID No. 251:

Sequence ID No. 251:
QSALTQPASVSGSPGQSITISCTGTSSDVGGYNYVSWYQQHPGKA
PKLMIYDVSNRPSGVPDRFSGSKSGTSASLAISGLQSEDEADYYC
HSYDSSLSHVFGTGTKVTVL,

(L-H2) an amino acid sequence having 80% or more identity to the amino acid sequence of Sequence ID No. 251, and
(L-H3) an amino acid sequence consisting of the amino acid sequence of Sequence ID No. 251 with deletion, substitution, insertion, and/or addition of one or several amino acids.

The amino acid sequence of (L-H1) above is a sequence that includes the amino acid sequences of (L1-H1) of the CDRL1, (L2-H1) of the CDRL2, and (L3-H1) of the CDRL3, for example. The amino acid sequence of (L-H2) above may be an amino acid sequence that includes the amino acid sequences of (L1-H1) of the CDRL1, (L2-H1) of the CDRL2, and (L3-H1) of the CDRL3 and that has 80% or more identity to the amino acid sequence of Sequence ID No. 251, for example. The amino acid sequence of (L-H3) above may be an amino acid sequence that includes the amino acid sequences of (L1-H1) of the CDRL1, (L2-H1) of the CDRL2, and (L3-H1) of the CDRL3 and that consists of the amino acid sequence of Sequence ID No. 251 with deletion, substitution, insertion, and/or addition of one or several amino acids, for example.

In the antibody of the present invention, the heavy-chain variable region is (H-A1) above, and the light-chain variable region is (L-H1) above, for example. The antibody that includes this combination is also referred to as an “antibody 18H-L22” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (L1) (Combination (L1))

The antibodies or the like of Combination (L1) are also referred to as an antibody 18H-K4 group, for example. In Combination (L1) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (L1) above includes the CDRL1, the CDRL2, and the CDRL3, the CDRL1 is a polypeptide that includes the amino acid sequence of (L1-I) below, the CDRL2 is a polypeptide that includes the amino acid sequence of (L2-I) below, and the CDRL3 is a polypeptide that includes the amino acid sequence of (L3-I) below.

(L1-I) An amino acid sequence of (L1-I1), (L1-I2), or (L1-I3) below:
(L1-I1) an amino acid sequence of Sequence ID No. 252 (QSVSSN),
(L1-I2) an amino acid sequence having 80% or more identity to the amino acid sequence of Sequence ID No. 252, and
(L1-I3) an amino acid sequence consisting of the amino acid sequence of Sequence ID No. 252 with deletion, substitution, insertion, and/or addition of one or several amino acids.

(L2-I) An amino acid sequence of (L2-I1), (L2-I2), or (L2-I3) below:
(L2-I1) an amino acid sequence of Sequence ID No. 253 (GAS),
(L2-I2) an amino acid sequence having 80% or more identity to the amino acid sequence of Sequence ID No. 253, and
(L2-I3) an amino acid sequence consisting of the amino acid sequence of Sequence ID No. 253 with deletion, substitution, insertion, and/or addition of one or several amino acids.

(L3-I) An amino acid sequence of (L3-I1), (L3-I2), or (L3-I3) below:
(L3-I1) an amino acid sequence of Sequence ID No. 254 (QQYNNWPPLYT),
(L3-I2) an amino acid sequence having 80% or more identity to the amino acid sequence of Sequence ID No. 254, and
(L3-I3) an amino acid sequence consisting of the amino acid sequence of Sequence ID No. 254 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (L1) above, the heavy-chain variable region of (HA) above includes a polypeptide consisting of the amino acid sequence of (H-A) above, for example. The light-chain variable region of (L1) above includes a polypeptide consisting of the amino acid sequence of (L-I) below, for example.

(L-I) An amino acid sequence of (L-I1), (L-I2), or (L-I3) below:
(L-I1) an amino acid sequence of Sequence ID No. 255:

Sequence ID No. 255:
EIVLTQSPGTLSLSPGERATLSCRASQSVSSNLAWYQQKPGQAPR
LLIYGASTRATGIPARFSGSGSGTEFTLTISSLQSEDFAVYYCQQ
YNNWPPLYTFGQGTKLEIK,

(L-I2) an amino acid sequence having 80% or more identity to the amino acid sequence of Sequence ID No. 255, and
(L-I3) an amino acid sequence consisting of the amino acid sequence of Sequence ID No. 255 with deletion, substitution, insertion, and/or addition of one or several amino acids.

The amino acid sequence of (L-I1) above is a sequence that includes the amino acid sequences of (L1-I1) of the CDRL1, (L2-I1) of the CDRL2, and (L3-I1) of the CDRL3, for example. The amino acid sequence of (L-I2) above may be an amino acid sequence that includes the amino acid sequences of (L1-I1) of the CDRL1, (L2-I1) of the CDRL2, and (L3-I1) of the CDRL3 and that has 80% or more identity to the amino acid sequence of Sequence ID No. 255, for example. The amino acid sequence of (L-I3) above may be an amino acid sequence that includes the amino acid sequences of (L1-I1) of the CDRL1, (L2-I1) of the CDRL2, and (L3-I1) of the CDRL3 and that consists of the amino acid sequence of Sequence ID No. 255 with deletion, substitution, insertion, and/or addition of one or several amino acids, for example.

In the antibody of the present invention, the heavy-chain variable region is (H-A1) above, and the light-chain variable region is (L-I1) above, for example. The antibody that includes this combination is also referred to as an “antibody 18H-K4” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LJ) (Combination (LJ))

The antibodies or the like of Combination (LJ) are also referred to as an antibody 18H-K5 group, for example. In Combination (LJ) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LJ) above includes the CDRL1, the CDRL2, and the CDRL3, the CDRL1 is a polypeptide that includes the amino acid sequence of (L1-J) below, the CDRL2 is a polypeptide that includes the amino acid sequence of (L2-J) below, and the CDRL3 is a polypeptide that includes the amino acid sequence of (L3-J) below.

(L1-J) An amino acid sequence of (L1-J1), (L1-J2), or (L1-J3) below:
(L1-J1) an amino acid sequence of Sequence ID No. 256 (QSVSSY),
(L1-J2) an amino acid sequence having 80% or more identity to the amino acid sequence of Sequence ID No. 256, and
(L1-J3) an amino acid sequence consisting of the amino acid sequence of Sequence ID No. 256 with deletion, substitution, insertion, and/or addition of one or several amino acids.

(L2-J) An amino acid sequence of (L2-J1), (L2-J2), or (L2-J3) below:
(L2-J1) an amino acid sequence of Sequence ID No. 257 (DAS),
(L2-J2) an amino acid sequence having 80% or more identity to the amino acid sequence of Sequence ID No. 257, and
(L2-J3) an amino acid sequence consisting of the amino acid sequence of Sequence ID No. 257 with deletion, substitution, insertion, and/or addition of one or several amino acids.

(L3-J) An amino acid sequence of (L3-J1), (L3-J2), or (L3-J3) below:
(L3-J1) an amino acid sequence of Sequence ID No. 258 (QQSYSTLLYT),
(L3-J2) an amino acid sequence having 80% or more identity to the amino acid sequence of Sequence ID No. 258, and
(L3-J3) an amino acid sequence consisting of the amino acid sequence of Sequence ID No. 258 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LJ) above, the heavy-chain variable region of (HA) above includes a polypeptide consisting of the amino acid sequence of (H-A) above, for example. The light-chain variable region of (LJ) above includes a polypeptide consisting of the amino acid sequence of (L-J) below, for example.

(L-J) An amino acid sequence of (L-J1), (L-J2), or (L-J3) below:
(L-J1) an amino acid sequence of Sequence ID No. 259:

Sequence ID No. 259:
EIVLTQSPGTLSLSPGERATLSCRASQSVSSYLAWYQQKPGQAPR
LLIYDASNRATGIPARFSGSGSGTDFTLTISSLEPEDFATYYCQQ
SYSTLLYTFGQGTKLEIK,

(L-J2) an amino acid sequence having 80% or more identity to the amino acid sequence of Sequence ID No. 259, and
(L-J3) an amino acid sequence consisting of the amino acid sequence of Sequence ID No. 259 with deletion, substitution, insertion, and/or addition of one or several amino acids.

The amino acid sequence of (L-J1) above is a sequence that includes the amino acid sequences of (L1-J1) of the CDRL1, (L2-J1) of the CDRL2, and (L3-J1) of the CDRL3, for example. The amino acid sequence of (L-J2) above may be an amino acid sequence that includes the amino acid sequences of (L1-J1) of the CDRL1, (L2-J1) of the CDRL2, and (L3-J1) of the CDRL3 and that has 80% or more identity to the amino acid sequence of Sequence ID No. 259, for example. The amino acid sequence of (L-J3) above may be an amino acid sequence that includes the amino acid sequences of (L1-J1) of the CDRL1, (L2-J1) of the CDRL2, and (L3-J1) of the CDRL3 and that consists of the amino acid sequence of Sequence ID No. 259 with deletion, substitution, insertion, and/or addition of one or several amino acids, for example.

In the antibody of the present invention, the heavy-chain variable region is (H-A1) above, and the light-chain variable region is (L-J1) above, for example. The antibody that includes this combination is also referred to as an “antibody 18H-K5” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LK) (Combination (LK))

The antibodies or the like of Combination (LK) are also referred to as an antibody 18H-K6 group, for example. In Combination (LK) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LK) above includes the CDRL1, the CDRL2, and the CDRL3, the CDRL1 is a polypeptide that includes the amino acid sequence of (L1-K) below, the CDRL2 is a polypeptide that includes the amino acid sequence of (L2-K) below, and the CDRL3 is a polypeptide that includes the amino acid sequence of (L3-K) below.

(L1-K) An amino acid sequence of (L1-K1), (L1-K2), or (L1-K3) below:
(L1-K1) an amino acid sequence of Sequence ID No. 260 (QSVSSY),
(L1-K2) an amino acid sequence having 80% or more identity to the amino acid sequence of Sequence ID No. 260, and
(L1-K3) an amino acid sequence consisting of the amino acid sequence of Sequence ID No. 260 with deletion, substitution, insertion, and/or addition of one or several amino acids.

(L2-K) An amino acid sequence of (L2-K1), (L2-K2), or (L2-K3) below:
(L2-K1) an amino acid sequence of Sequence ID No. 261 (DAS),
(L2-K2) an amino acid sequence having 80% or more identity to the amino acid sequence of Sequence ID No. 261, and
(L2-K3) an amino acid sequence consisting of the amino acid sequence of Sequence ID No. 261 with deletion, substitution, insertion, and/or addition of one or several amino acids.

(L3-K) An amino acid sequence of (L3-K1), (L3-K2), or (L3-K3) below:
(L3-K1) an amino acid sequence of Sequence ID No. 262 (QQYDSLPLT),
(L3-K2) an amino acid sequence having 80% or more identity to the amino acid sequence of Sequence ID No. 262, and
(L3-K3) an amino acid sequence consisting of the amino acid sequence of Sequence ID No. 262 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LK) above, the heavy-chain variable region of (HA) above includes a polypeptide consisting of the amino acid sequence of (H-A) above, for example. The light-chain variable region of (LK) above includes a polypeptide consisting of the amino acid sequence of (L-K) below, for example.

(L-K) An amino acid sequence of (L-K1), (L-K2), or (L-K3) below:
(L-K1) an amino acid sequence of Sequence ID No. 263:

Sequence ID No. 263:
EIVLTRSPATLSLSPGERATLSCRASQSVSSYLAWYQQKPGQAPR
LLIYDASNRATGISARFSGSGSGTEFTLTISSLQSEDIATYYCQQ
YDSLPLTFGGGTKLEIK.

(L-K2) an amino acid sequence having 80% or more identity to the amino acid sequence of Sequence ID No. 263, and
(L-K3) an amino acid sequence consisting of the amino acid sequence of Sequence ID No. 263 with deletion, substitution, insertion, and/or addition of one or several amino acids.

The amino acid sequence of (L-K1) above is a sequence that includes the amino acid sequences of (L1-K1) of the CDRL1, (L2-K1) of the CDRL2, and (L3-K1) of the CDRL3, for example. The amino acid sequence of (L-K2) above may be an amino acid sequence that includes the amino acid sequences of (L1-K1) of the CDRL1, (L2-K1) of the CDRL2, and (L3-K1) of the CDRL3 and that has 80% or more identity to the amino acid sequence of Sequence ID No. 263, for example. The amino acid sequence of (L-K3) above may be an amino acid sequence that includes the amino acid sequences of (L1-K1) of the CDRL1, (L2-K1) of the CDRL2, and (L3-K1) of the CDRL3 and that consists of the amino acid sequence of Sequence ID No. 263 with deletion, substitution, insertion, and/or addition of one or several amino acids, for example.

In the antibody of the present invention, the heavy-chain variable region is (H-A1) above, and the light-chain variable region is (L-K1) above, for example. The antibody that includes this combination is also referred to as an “antibody 18H-K6” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LL) (Combination (LL))

The antibodies or the like of Combination (LL) are also referred to as an antibody 18H-K9 group, for example. In Combination (LL) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LL) above includes the CDRL1, the CDRL2, and the CDRL3, the CDRL1 is a polypeptide that includes the amino acid sequence of (L1-L) below, the CDRL2 is a polypeptide that includes the amino acid sequence of (L2-L) below, and the CDRL3 is a polypeptide that includes the amino acid sequence of (L3-L) below.

(L1-L) An amino acid sequence of (L1-L1), (L1-L2), or (L1-L3) below:
(L1-L1) an amino acid sequence of Sequence ID No. 264 (QTISASS),
(L1-L2) an amino acid sequence having 80% or more identity to the amino acid sequence of Sequence ID No. 264, and
(L1-L3) an amino acid sequence consisting of the amino acid sequence of Sequence ID No. 264 with deletion, substitution, insertion, and/or addition of one or several amino acids.

(L2-L) An amino acid sequence of (L2-L1), (L2-L2), or (L2-L3) below:
(L2-L1) an amino acid sequence of Sequence ID No. 265 (GAS),
(L2-L2) an amino acid sequence having 80% or more identity to the amino acid sequence of Sequence ID No. 265, and
(L2-L3) an amino acid sequence consisting of the amino acid sequence of Sequence ID No. 265 with deletion, substitution, insertion, and/or addition of one or several amino acids.

(L3-L) An amino acid sequence of (L3-L1), (L3-L2), or (L3-L3) below:
(L3-L1) an amino acid sequence of Sequence ID No. 266 (QQFNEWPLT),
(L3-L2) an amino acid sequence having 80% or more identity to the amino acid sequence of Sequence ID No. 266, and
(L3-L3) an amino acid sequence consisting of the amino acid sequence of Sequence ID No. 266 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LL) above, the heavy-chain variable region of (HA) above includes a polypeptide consisting of the amino acid sequence of (H-A) above, for example. The light-chain variable region of (LL) above includes a polypeptide consisting of the amino acid sequence of (L-L) below, for example.

(L-L) An amino acid sequence of (L-L1), (L-L2), or (L-L3) below:
(L-L1) an amino acid sequence of Sequence ID No. 267:

Sequence ID No. 267:
EIVLTQSPATLSLSPGERATVSCRPSQTISASSVAWYQQKAGQAP
RLLIYGASSRATGIPDRFSGSGSGTDFTLTISRLEPEDFAVYYCQ
QFNEWPLTFGGGTKVEIK

(L-L2) an amino acid sequence having 80% or more identity to the amino acid sequence of Sequence ID No. 267, and
(L-L3) an amino acid sequence consisting of the amino acid sequence of Sequence ID No. 267 with deletion, substitution, insertion, and/or addition of one or several amino acids.

The amino acid sequence of (L-L1) above is a sequence that includes the amino acid sequences of (L1-L1) of the CDRL1, (L2-L1) of the CDRL2, and (L3-L1) of the CDRL3, for example. The amino acid sequence of (L-L2) above may be an amino acid sequence that includes the amino acid sequences of (L1-L1) of the CDRL1, (L2-L1) of the CDRL2, and (L3-L1) of the CDRL3 and that has 80% or more identity to the amino acid sequence of Sequence ID No. 267, for example. The amino acid sequence of (L-L3) above may be an amino acid sequence that includes the amino acid sequences of (L1-L1) of the CDRL1, (L2-L1) of the CDRL2, and (L3-L1) of the CDRL3 and that consists of the amino acid sequence of Sequence ID No. 267 with deletion, substitution, insertion, and/or addition of one or several amino acids, for example.

In the antibody of the present invention, the heavy-chain variable region is (H-A1) above, and the light-chain variable region is (L-L1) above, for example. The antibody that includes this combination is also referred to as an “antibody 18H-K9” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

### Combination of (HA) and (LM) (Combination (LM))

The antibodies or the like of Combination (LM) are also referred to as an antibody 18H-K10 group, for example. In Combination (LM) above, the heavy-chain variable region of (HA) above includes the CDRH1, the CDRH2, and the CDRH3, the CDRH1 is a polypeptide that includes the amino acid sequence of (H1-A) above, the CDRH2 is a polypeptide that includes the amino acid sequence of (H2-A) above, and the CDRH3 is a polypeptide that includes the amino acid sequence of (H3-A) above. The light-chain variable region of (LM) above includes the CDRL1, the CDRL2, and the CDRL3, the CDRL1 is a polypeptide that includes the amino acid sequence of (L1-M) below, the CDRL2 is a polypeptide that includes the amino acid sequence of (L2-M) below, and the CDRL3 is a polypeptide that includes the amino acid sequence of (L3-M) below.

(L1-M) An amino acid sequence of (L1-M1), (L1-M2), or (L1-M3) below:
(L1-M1) an amino acid sequence of Sequence ID No. 268 (QSVSSN),
(L1-M2) an amino acid sequence having 80% or more identity to the amino acid sequence of Sequence ID No. 268, and
(L1-M3) an amino acid sequence consisting of the amino acid sequence of Sequence ID No. 268 with deletion, substitution, insertion, and/or addition of one or several amino acids.

(L2-M) An amino acid sequence of (L2-M1), (L2-M2), or (L2-M3) below:
(L2-M1) an amino acid sequence of Sequence ID No. 269 (GAS),
(L2-M2) an amino acid sequence having 80% or more identity to the amino acid sequence of Sequence ID No. 269, and
(L2-M3) an amino acid sequence consisting of the amino acid sequence of Sequence ID No. 269 with deletion, substitution, insertion, and/or addition of one or several amino acids.

(L3-M) An amino acid sequence of (L3-M1), (L3-M2), or (L3-M3) below:
(L3-M1) an amino acid sequence of Sequence ID No. 270 (QQYGSSPDIFT),
(L3-M2) an amino acid sequence having 80% or more identity to the amino acid sequence of Sequence ID No. 270, and
(L3-M3) an amino acid sequence consisting of the amino acid sequence of Sequence ID No. 270 with deletion, substitution, insertion, and/or addition of one or several amino acids.

The “identity” as used for the CDRs refers to 80% or more, 85% or more, 90% or more, 95% or more, 96% or more, 97% or more, 98% or more, or 99% or more identity, for example.

The term “one or several” as used for the CDRs regarding substitution and the like refers to 1 to 5, 1 to 4, 1 to 3, 1 or 2, or 1, for example.

In Combination (LM) above, the heavy-chain variable region of (HA) above includes a polypeptide consisting of the amino acid sequence of (H-A) above, for example. The light-chain variable region of (LM) above includes a polypeptide consisting of the amino acid sequence of (L-M) below, for example.

(L-M) An amino acid sequence of (L-M1), (L-M2), or (L-M3) below:
(L-M1) an amino acid sequence of Sequence ID No. 271:

Sequence ID No. 271:
EIVLTQSPATLSLSPGERATLSCRASQSVSSNLAWYQQKLGQAPR
LLIYGASSRATGIPDRFSGSGSGTDFTLTISRLEPEDFAVYYCQQ
YGSSPDIFTFGPGTKVDIK

(L-M2) an amino acid sequence having 80% or more identity to the amino acid sequence of Sequence ID No. 271, and
(L-M3) an amino acid sequence consisting of the amino acid sequence of Sequence ID No. 271 with deletion, substitution, insertion, and/or addition of one or several amino acids.

The amino acid sequence of (L-M1) above is a sequence that includes the amino acid sequences of (L1-M1) of the CDRL1, (L2-M1) of the CDRL2, and (L3-M1) of the CDRL3, for example. The amino acid sequence of (L-M2) above may be an amino acid sequence that includes the amino acid sequences of (L1-M1) of the CDRL1, (L2-M1) of the CDRL2, and (L3-M1) of the CDRL3 and that has 80% or more identity to the amino acid sequence of Sequence ID No. 271, for example. The amino acid sequence of (L-M3) above may be an amino acid sequence that includes the amino acid sequences of (L1-M1) of the CDRL1, (L2-M1) of the CDRL2, and (L3-M1) of the CDRL3 and that consists of the amino acid sequence of Sequence ID No. 271 with deletion, substitution, insertion, and/or addition of one or several amino acids, for example.

In the antibody of the present invention, the heavy-chain variable region is (H-A1) above, and the light-chain variable region is (L-M1) above, for example. The antibody that includes this combination is also referred to as an “antibody 18H-K10” hereinafter.

The “identity” as used for the polypeptide of the light-chain variable region refers to 80% or more, 85% or more, 90% or more, 95% or more, 96% or more, 97% or more, 98% or more, or 99% or more identity, for example.

The term “one or several” as used for the polypeptide of the light-chain variable region regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

In the present invention, the amino acid sequences of Sequence ID Nos. 216 to 271 are human-derived amino acid sequences, for example.

The binding of the antibody of the present invention or the like to CD19 can be confirmed using a technique for detecting the binding of an antibody to an antigen such as surface plasmon resonance (SPR) or flow cytometry, for example.

The antibody of the present invention or the like may be labeled with a labeling substance or immobilized on a carrier, a porous body, or the like, for example. The descriptions above can be applied to the above-mentioned labeling and immobilization.

There is no particular limitation on a method for manufacturing the antibody of the present invention or the like, and the antibody or the like can be manufactured using genetic engineering techniques based on the amino acid sequence information described above, for example. Specifically, the antibody or the like can be manufactured as follows, for example. It should be noted that the present invention is not limited to this example.

First, a vector that includes nucleic acid sequences coding for the amino acid sequences of the above-mentioned regions, heavy chain, and/or light chain in the antibody of the present invention or the like is introduced into a host, and thus a transformant is obtained. Then, the transformant is cultured, a fraction containing an antibody capable of binding to CD19 is collected, and the antibody is isolated or purified from the obtained collected fraction.

The descriptions above can be applied to the above-mentioned vector and the above-mentioned method for culturing a transformant into which the vector has been introduced.

In the present invention, the antibody is a monoclonal antibody, for example. Examples of the monoclonal antibody include monoclonal antibodies obtained through immunization of an animal, chimeric antibodies, humanized antibodies, and human antibodies (also referred to as “fully human antibodies”). The descriptions above can be applied to the above-mentioned chimeric antibodies and humanized antibodies.

The antibody of the present invention or the like can also be prepared through immunization of an animal with an antigen, for example. An example of the antigen is the CD19 protein. It is preferable to repeat the immunization with the antigen a plurality of times. The peptide fragment may be a peptide fragment constituted by only an antigenic determinant (epitope) or a peptide fragment that includes the antigenic determinant, for example. The descriptions above can be applied to the above-mentioned method for manufacturing a monoclonal antibody.

In the second antibody of the present invention or the like, the light-chain variable region of (L) above may be a light-chain variable region of (L) below.

(L) A light-chain variable region that includes
a light-chain complementarity determining region (CDRL) 1, a CDRL2, and a CDRL3,
wherein the CDRL1 is a polypeptide that includes an amino acid sequence of (L1),
the CDRL2 is a polypeptide that includes an amino acid sequence of (L2),
the CDRL3 is a polypeptide that includes an amino acid sequence of (L3),
the amino acid sequences of (L1), (L2), and (L3) are as follows:
(L1) an amino acid sequence of (L1-1), (L1-2), or (L1-3) below:
   (L1-1) any one of amino acid sequences of CDRL1 shown in Conditions (L1) to (L3) and (L4) below,
   (L1-2) an amino acid sequence having 80% or more identity to the amino acid sequence of (L1-1), and
   (L1-3) an amino acid sequence consisting of the amino acid sequence of (L1-1) with deletion, substitution, insertion, and/or addition of one or several amino acids;
(L2) an amino acid sequence of (L2-1), (L2-2), or (L2-3) below:
   (L2-1) any one of amino acid sequences of CDRL2 shown in Conditions (L1) to (L3) and (L4) below,
   (L2-2) an amino acid sequence having 80% or more identity to the amino acid sequence of (L2-1), and
   (L2-3) an amino acid sequence consisting of the amino acid sequence of (L2-1) with deletion, substitution, insertion, and/or addition of one or several amino acids;
(L3) an amino acid sequence of (L3-1), (L3-2), or (L3-3) below:
   (L3-1) any one of amino acid sequences of CDRL3 shown in Conditions (L1) to (L3) and (L4) below,
   (L3-2) an amino acid sequence having 80% or more identity to the amino acid sequence of (L3-1), and
   (L3-3) an amino acid sequence consisting of the amino acid sequence of (L3-1) with deletion, substitution, insertion, and/or addition of one or several amino acids

Condition (L1) is as follows:
the CDRL1 is KLGDKY (Sequence ID No. 220),
the CDRL2 is QDS (Sequence ID No. 221), and
the CDRL3 is QAWDSSTHV (Sequence ID No. 222),

Condition (L2) is as follows:
the CDRL1 is WSNIGDDH (Sequence ID No. 228),
the CDRL2 is DTS (Sequence ID No. 229), and
the CDRL3 is GTWESSLSGVV (Sequence ID No. 230),

Condition (L3) is as follows:
the CDRL1 is X₁SX₂X₃GX₄X₅NY (Sequence ID No. 317),
the CDRL2 is DX₆X₇ (Sequence ID No. 318),
the CDRL3 is X₈X₉X₁₀X₁₁X₁₂SX₁₃X₁₄X₁₅X₁₆X₁₇ (Sequence ID No. 319),
X₁ is S or T,
X₂ is D or N,
X₃ is V, I, L, or A,
X₄ is G, T, V, or N,
X₅ is not present, or is Y, T, or S,
X₆ is V or N,
X₇ is S, I, or V,
X₈ is not present, or is G, A, or H,
X₉ is T, S, or A,
X₁₀ is W, Y, or F,
X₁₁ is A or D,
X₁₂ is T, G, D, or S,
X₁₃ is L or Y,
X₁₄ is T, N, or S,
X₁₅ is A, F, G, or H,
X₁₆ is V or I, and
X₁₇ is not present or is V, and

Condition (L4) is as follows:
the CDRL1 is QX₁X₂SX₃SX₄ (Sequence ID No. 320),
the CDRL2 is GAS (Sequence ID No. 253) or DAS (Sequence ID No. 257),
the CDRL3 is QQX₅X₆X₇X₈X₉X₁₀X₁₁LX₁₂T (Sequence ID No. 321),
X₁ is S, T, N, or Q,
X₂ is V, I, L, or A,
X₃ is not present, or is A, I, V, L, or G,
X₄ is N, Y, or S,
X₅ is not present or is S,
X₆ is Y, F, or W,
X₇ is N, S, D, or G,
X₈ is N, T, S, or E,
X₉ is W, L, or S,
X₁₀ is not present or is P,
X₁₁ is not present, or is P or D, and
X₁₂ is not present, or is Y, F, or W.

Regarding the light-chain variable region of (L) above, (L1-1), (L2-1), and (L3-1) meet the same condition or different conditions of Conditions (L1) to (L4), and preferably meet the same condition.

In Condition (L3), it is preferable that X₃ is V or I. It is preferable that X₅ is not present, or is Y or T. It is preferable that X₁₀ is W or Y.

In Condition (L4), it is preferable that X₁ is S or T. It is preferable that X₂ is V or I. It is preferable that X₆ is Y or F. It is preferable that X₁₂ is not present, or is Y or F.

An example of light-chain variable regions that meet Condition (L1) above include the light-chain variable region of (LA) above.

An example of light-chain variable regions that meet Condition (L2) above include the light-chain variable region of (LC) above.

Examples of light-chain variable regions that meet Condition (L3) above include the light-chain variable regions of (LB), (LE), (LF), (LG), and (LH) above.

Examples of light-chain variable regions that meet Condition (L4) above include the light-chain variable regions of (LI), (LJ), (LK), (LL), and (LM) above.

### Gene, Expression Vector, and Transformant

The coding gene of the present invention is a gene coding for an antibody against a complex of HLA-A*02:01 and NY-ESO-1₁₅₇₋₁₆₅ or an antigen-binding fragment thereof, or an antibody against CD19 or an antigen-binding fragment (also collectively referred to as “an antibody of the present invention or an antigen-binding fragment thereof” or “an antibody of the present invention or the like” hereinafter), and includes a nucleic acid (polynucleotide) coding for the amino acid sequence of the first antibody of the present invention or the antigen-binding fragment, or the second antibody of the present invention or the antigen-binding fragment.

The above-described antibody of the present invention or the like can be obtained through the expression of the coding gene of the present invention. There is no particular limitation on the sequence of the coding gene of the present invention, and it is sufficient that the sequence thereof codes for the amino acid sequence of the antibody of the present invention or the like. The sequence thereof may be a sense sequence or an antisense sequence.

The expression vector of the present invention is an expression vector for expressing an antibody against A2/NY-ESO-1₁₅₇ or an antigen-binding fragment thereof, or an antibody against CD19 or an antigen-binding fragment, and includes the coding gene of the present invention. In the expression vector, the coding gene is coupled to the linking vector such that the antibody of the present invention or the antigen-binding fragment thereof can be expressed. It is sufficient that the expression vector of the present invention can express the antibody of the present invention or the like, and there is no particular limitation on the other configurations.

The expression vector of the present invention may be an expression vector that includes a nucleic acid sequence coding for the heavy-chain variable region and a nucleic acid sequence coding for the light-chain variable region, or a set of an expression vector that includes a nucleic acid sequence coding for the heavy-chain variable region and an expression vector that includes a nucleic acid sequence coding for the light-chain variable region, for example. The expression vector of the present invention can be prepared by coupling the coding gene of the present invention to a linking vector, for example. The descriptions of the expression vector in the CAR library of the present invention can be applied to the linking vector, for example. There is no particular limitation on the type of linking vector to which the coding gene is to be coupled, and examples thereof include pUC and pMX. The linking vector can also be selected as appropriate in accordance with a host into which the expression vector is to be introduced, for example. There is no particular limitation on the host, and examples thereof include mammalian cells such as CHO cells.

The transformant of the present invention is a transformant that expresses the antibody of the present invention or the like, and includes a host and the coding gene of the present invention. It is sufficient that the transformant of the present invention includes the coding gene of the present invention so as to be capable of expressing the coding gene. It is preferable that the transformant includes the expression vector of the present invention, for example. There is no particular limitation on a method for introducing the expression vector into the host, and a known method can be employed.

The descriptions of the CAR library of the present invention, the first screening method, the first antibody or the like, the second antibody of the present invention or the like can be applied to the gene, the expression vector, and the transformant of the present invention, for example.

### First Chimeric Antigen Receptor

As described above, the first chimeric antigen receptor (CAR) of the present invention includes an antigen-binding domain, a transmembrane domain, and an intracellular signaling domain, and the antigen-binding domain includes the antibody of the present invention or the antigen-binding fragment thereof. The first CAR of the present invention is characterized in that the antigen-binding domain includes the first antibody of the present invention or the like, and there is no particular limitation on the other configurations and conditions. The descriptions of the CAR library, the first screening method, the first antibody or the like, the second antibody or the like, the gene, the expression vector, and the transformant of the present invention can be applied to the first CAR of the present invention. The first CAR of the present invention can be favorably used as a CAR for CAR-T cells against A2/NY-ESO-1₁₅₇-expressing cancer cells, for example.

The antigen-binding domain may be the first antibody of the present invention or the antigen-binding fragment of the first antibody of the present invention, for example. The antigen-binding fragment is preferably a single-chain antibody (scFv). If the antigen-binding domain is an scFv, the heavy-chain variable region and the light-chain variable region are arranged in this order from the C terminus or N terminus in the antigen-binding domain.

A specific example of the antigen-binding domain is a polypeptide of (BD) below.
(BD) A polypeptide of (BD1), (BD2), or (BD3) below:
   (BD1) a polypeptide consisting of the amino acid sequence of any one of Sequence ID Nos. 98 to 117,
   (BD2) a polypeptide consisting of an amino acid sequence having 80% or more identity to the amino acid sequence of any one of Sequence ID Nos. 98 to 117, the polypeptide being capable of binding to A2/NY-ESO-1₁₅₇, and
   (BD3) a polypeptide consisting of an amino acid sequence consisting of the amino acid sequence of any one of Sequence ID Nos. 98 to 117 with deletion, substitution, insertion, and/or addition of one or several amino acids, the polypeptide being capable of binding to A2/NY-ESO-1₁₅₇.

In the polypeptide of (BD1) above, each of the amino acid sequences of Sequence ID Nos. 98 to 117 is an amino acid sequence that includes a combination of the heavy-chain variable region and the light-chain variable region that corresponds to any one of Combinations (1) to (18) for the first antibody of the present invention or the like, for example. Regarding the amino acid sequences of the heavy-chain variable region and the light-chain variable region in each of the amino acid sequences of Sequence ID Nos. 98 to 117, the amino acid sequences that include the combinations of the heavy-chain variable region and the light-chain variable region that correspond to Combinations (1) to (18) for the first antibody of the present invention or the like can be referred to, for example. In the polypeptides of (BD2) and (BD3) above, the amino acid sequences that correspond to the CDRH1, the CDRH2, the CDRH3, the CDRL1, the CDRL2, and the CDRL3 are conserved in the amino acid sequences of Sequence ID Nos. 98 to 117, for example. Regarding the amino acid sequences of the CDRs in the amino acid sequences of Sequence ID Nos. 98 to 117, the amino acid sequences of the CDRs included in the amino acid sequences that include the combinations of the heavy-chain variable region and the light-chain variable region that correspond to Combinations (1) to (18) for the first antibody of the present invention or the like can be referred to, for example.

Antigen-Binding Domain of H1-3M4E5L
(Sequence ID No. 98)
QVQLQESGPGLVKPSDTLSLTCLVSGGSISSNYWSWIRQAPGKGLEWIGHVSY
SGSTNYNPSLKSRVTISVDTSKNQFSLKLSSVTAADTAVYYCARESYYYYGMDVWGQ
GTTVTVSSGSTSGSGKPGSGEGSTKGQSELTQPRSVSGSPGQSVTISCTGTSRDVGGYN
YVSWYQQHPGKAPKLIIHDVIERSSGVPDRFSGSKSGNTASLTISGLQAEDEADYYCWS
FAGSYYVFGTGTDVTVL
Antigen-Binding Domain of K52-H1
(Sequence ID No. 99)
DIQMTQSPSAMSASVGDRVTITCRASQSISSYLNWYQQKPGKAPKLLIYAASS
LQSGVPSRFSGSGSGTDFTLTISSLQPEDFATYYCQQYYSTPQTFGPGTKVDIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSS
Antigen-Binding Domain of K73-H1
(Sequence ID No. 100)
DIQMTQSPSSLSASVGDRVTITCRASQSISSYLNWYQQKAGKAPKLLIYAASSL
QSGVPSRFSGSGSGTDFTLTISCLOSEDVATYYCQQYESYRRSFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSS
Antigen-Binding Domain of K121-H1
(Sequence ID No. 101)
DIQMTQSPSSLSASVGDRVTITCRASQSISSYLNWYQQKPGKAPKLLIYAASSL
QSGVPSRFSGSGSGTDFTLTISCLOSEDFATYYCQQYNSYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSS
Antigen-Binding Domain of K124-H1
(Sequence ID No. 102)
DIQMTQSPSSLSASVGDRVTITCRASQSISSYLNWYQQKPGKAPKLLIYAASRL
ESGVPSRFSGSGSGTDFTLTISCLOSEDFATYYCQQYNSYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSS
Antigen-Binding Domain of K125-H1
(Sequence ID No. 103)
DIQMTQSPSSLSASVGDRVTITCRASQSISSYLNWYQQKPGKAPKLLLYAASRL
ESGVPSRFSGSGSGTEFTLTISSLQPDDFATYYCQQYNSYSPCTFGPGTKVDIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSS
Antigen-Binding Domain of K131-H1
(Sequence ID No. 104)
DIQMTQSPSSLSASVGDRVSITCRASQDISRYLNWYQQKPGKAPKLLLYAASR
LESGVPSRFSGSGSGTDFTLTITSLQPDDFATYYCQQYDNLITFGQGTRLEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSS
Antigen-Binding Domain of K145-H1
(Sequence ID No. 105)
DIQMTQSPSSLSASVGDRVSITCRASQDISRYLNWYQQKPGKAPKLLLYAASR
LESGVPSRFSGSGSGTDFTLTITSLQPDDFATYYCQQYNSYSRTFGQGTKVEIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSS
Antigen-Binding Domain of K151-H1
(Sequence ID No. 106)
DIQMTQSPSSLSASVGDRVTITCRASQSISSYLNWYQQKPGKAPKLLIYAASSL
QSGVPSRFSGSGSGTDFTLTITSLQPDDFATYYCQQYDNLITFGQGTRLEIKGSTSGSGK
PGSGEGSTKGQVQLQESGPGLVKPSDTLSLTCLVSGGSISSNYWSWIRQAPGKGLEWIG
HVSYSGSTNYNPSLKSRVTISVDTSKNQFSLKLSSVTAADTAVYYCARESYYYYGMDV
WGQGTTVTVSS
Antigen-Binding Domain of K160-H1
(Sequence ID No. 107)
EIVLTQSPATLSVSPGERATLSCRASQSVSSNLAWYQQKPGQAPRLLIYGASTR
ATGIPARFSGSGSGTEFTLTISRLEPEDFATYYCQQYNSYSRTFGQGTKVEIKGSTSGSGK
PGSGEGSTKGQVQLQESGPGLVKPSDTLSLTCLVSGGSISSNYWSWIRQAPGKGLEWIG
HVSYSGSTNYNPSLKSRVTISVDTSKNQFSLKLSSVTAADTAVYYCARESYYYYGMDV
WGQGTTVTVSS
Antigen-Binding Domain of K173-H1
(Sequence ID No. 108)
DIQMTQSPSSLSASVGDRVTITCRASQSISSYLNWYQQKAGKAPKLLIYAASSL
QSGVPSRFSGSGSGTDFTLTISCLOSEDVATYYCQQYESYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSS
Antigen-Binding Domain of L1-3M4E5H
(Sequence ID No. 109)
QSALTQPPSASGSPGQSVTISCTGTSSDVGGYDFVSWYQQHPGEAPKLLVYDV
NNRPSGVSNRFSGSKSGNTASLTISGLQAEDEGDYYCSSYAGSNSVFGTGTKVTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSS
Antigen-Binding Domain of 3M4E5H-L1
(Sequence ID No. 110)
EVQLLESGGGLVQPGGSLRLSCAASGFTFSTYQMSWVRQAPGKGLEWVSGIV
SSGGSTAYADSVKGRFTISRDNSKNTLYLQMNSLRAEDTAVYYCAGELLPYYGMDVW
GQGTTVTVSSGSTSGSGKPGSGEGSTKGQSALTQPPSASGSPGQSVTISCTGTSSDVGG
YDEVSWYQQHPGEAPKLLVYDVNNRPSGVSNRFSGSKSGNTASLTISGLQAEDEGDYY
CSSYAGSNSVFGTGTKVTVL
Antigen-Binding Domain of L66-3M4E5H
(Sequence ID No. 111)
QSALTQPASVSGSPGQSITISCTGTSSDVGGYEFVSWYQQHPGSAPKLIIYDVIE
RPFGVSYRFSASKSGNTASLTISGLQGEDEADYFCSSYTSSSTYVFGTGTKVTVLGSTSG
SGKPGSGEGSTKGEVQLLESGGGLVQPGGSLRLSCAASGFTFSTYQMSWVRQAPGKG
LEWVSGIVSSGGSTAYADSVKGRFTISRDNSKNTLYLQMNSLRAEDTAVYYCAGELLP
YYGMDVWGQGTTVTVSS
Antigen-Binding Domain of L73-3M4E5H
(Sequence ID No. 112)
QSALTQPASVSGSPGQSITISCTGTGSDVGAYDYVSWYQHHPGRAPRLIIRDVS
VRPSGVPDRFSGSKSGNTASLTISGLQAEDEADYYCSSYSGSSTWVFGGGTKLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSS
Antigen-Binding Domain of L80-3M4E5H
(Sequence ID No. 113)
QSALTQPASVSGSPGQSITISCTGTSSDVGSYNLVSWYQQHPGKAPKLMIYDVS
NRPSGVSYRFSGSKSGNTASLTISGLQAEDEADYYCSSYTSSSTFAVFGGGTQLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSS
Antigen-Binding Domain of L88-3M4E5H
(Sequence ID No. 114)
QSALPQPASVSGSPGQSVTISCTGTSSDVGGYNYVSWYQQHPGKAPKLMIYD
VSKRPSGVPDRFSGSKSGNTASLTVSGLQAEDEADYFCCSYAGGYYVFGTGTKLTVLG
STSGSGKPGSGEGSTKGEVQLLESGGGLVQPGGSLRLSCAASGFTFSTYQMSWVRQAP
GKGLEWVSGIVSSGGSTAYADSVKGRFTISRDNSKNTLYLQMNSLRAEDTAVYYCAGE
LLPYYGMDVWGQGTTVTVSS
Antigen-Binding Domain of L102-3M4E5H
(Sequence ID No. 115)
QSALTQPPSASGSPGQSVTISCTGTSSDVGGYNYVSWYQQHPGKAPKLMIYD
VSKRPSGVPDRFSGSKSGNTASLTISGLQTEDEADYYCSSYAGSGSTPFVFGTGTKLTVL
GSTSGSGKPGSGEGSTKGEVQLLESGGGLVQPGGSLRLSCAASGFTFSTYQMSWVRQA
PGKGLEWVSGIVSSGGSTAYADSVKGRFTISRDNSKNTLYLQMNSLRAEDTAVYYCAG
ELLPYYGMDVWGQGTTVTVSS
Antigen-Binding Domain of L124-3M4E5H
(Sequence ID No. 116)
QSALTQPASVSGSPGQSITISCTGTSSDVGGYNYVSWYQQHPGKAPKLMIYDV
SNRPSRVPDRFAGSKSGNTASLTISGLQAEDEADYYCCSYAGRRYVFGTGTKLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSS
Antigen-Binding Domain of H73-3M4E5L
(Sequence ID No. 117)
QLQLQESGPGLVKPSQTLSLTCTVSGGSISSYYWSWIRQPPGKGLEWIGEINHS
GSTNYNPSLKSRVTISVDTSKNQFSLKLSSVTAADTAVYYCARCPIYYYGMDVWGQGT
TVTVSSGSTSGSGKPGSGEGSTKGQSELTQPRSVSGSPGQSVTISCTGTSRDVGGYNYV
SWYQQHPGKAPKLIIHDVIERSSGVPDRFSGSKSGNTASLTISGLQAEDEADYYCWSFA
GSYYVFGTGTDVTVL

In the polypeptide of (BD1) above, the amino acid sequences of the heavy-chain variable region and the light-chain variable region may also be arranged in the reverse order. In a specific example, the polypeptide of (BD1) above may be a polypeptide obtained by exchanging the amino acid sequences of the heavy-chain variable region and the light-chain variable region in the amino acid sequences of Sequence ID Nos. 98 to 117.

The “identity” as used for the polypeptide of (BD2) above refers to 80% or more, 85% or more, 90% or more, 95% or more, 96% or more, 97% or more, 98% or more, or 99% or more identity, for example.

The term “one or several” as used for the polypeptide of (BD3) above regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

The descriptions of the first transmembrane domain and the first intracellular signaling domain of the CAR library of the present invention can be respectively applied to the above-mentioned transmembrane domain and intracellular signaling domain, for example.

A specific example of the first CAR of the present invention is a polypeptide of (C) below, for example.

(C) A polypeptide of (C1), (C2), or (C3) below:
(C1) a polypeptide consisting of the amino acid sequence of any one of Sequence ID Nos. 118 to 137,
(C2) a polypeptide consisting of an amino acid sequence having 80% or more identity to the amino acid sequence of any one of Sequence ID Nos. 118 to 137, the polypeptide being capable of binding to A2/NY-ESO-1₁₅₇, and
(C3) a polypeptide consisting of an amino acid sequence consisting of the amino acid sequence of any one of Sequence ID Nos. 118 to 137 with deletion, substitution, insertion, and/or addition of one or several amino acids, the polypeptide being capable of binding to A2/NY-ESO-1₁₅₇.

H1-3M4E5L CAR
(Sequence ID No. 118)
QVQLQESGPGLVKPSDTLSLTCLVSGGSISSNYWSWIRQAPGKGLEWIGHVSY
SGSTNYNPSLKSRVTISVDTSKNQFSLKLSSVTAADTAVYYCARESYYYYGMDVWGQ
GTTVTVSSGSTSGSGKPGSGEGSTKGQSELTQPRSVSGSPGQSVTISCTGTSRDVGGYN
YVSWYQQHPGKAPKLIIHDVIERSSGVPDRFSGSKSGNTASLTISGLQAEDEADYYCWS
FAGSYYVFGTGTDVTVLAAAIEVMYPPPYLDNEKSNGTIIHVKGKHLCPSPLFPGPSKP
FWVLVVVGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDAL
HMQALPPR
K52-H1 CAR
(Sequence ID No. 119)
DIQMTQSPSAMSASVGDRVTITCRASQSISSYLNWYQQKPGKAPKLLIYAASS
LQSGVPSRFSGSGSGTDFTLTISSLQPEDFATYYCQQYYSTPQTFGPGTKVDIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSSAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPR
K73-H1 CAR
(Sequence ID No. 120)
DIQMTQSPSSLSASVGDRVTITCRASQSISSYLNWYQQKAGKAPKLLIYAASSL
QSGVPSRFSGSGSGTDFTLTISCLOSEDVATYYCQQYESYRRSFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K121-H1 CAR
(Sequence ID No. 121)
DIQMTQSPSSLSASVGDRVTITCRASQSISSYLNWYQQKPGKAPKLLIYAASSL
QSGVPSRFSGSGSGTDFTLTISCLOSEDFATYYCQQYNSYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K124-H1 CAR
(Sequence ID No. 122)
DIQMTQSPSSLSASVGDRVTITCRASQSISSYLNWYQQKPGKAPKLLIYAASRL
ESGVPSRFSGSGSGTDFTLTISCLOSEDFATYYCQQYNSYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K125-H1 CAR
(Sequence ID No. 123)
DIQMTQSPSSLSASVGDRVTITCRASQSISSYLNWYQQKPGKAPKLLLYAASRL
ESGVPSRFSGSGSGTEFTLTISSLQPDDFATYYCQQYNSYSPCTFGPGTKVDIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSSAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPR
K131-H1 CAR
(Sequence ID No. 124)
DIQMTQSPSSLSASVGDRVSITCRASQDISRYLNWYQQKPGKAPKLLLYAASR
LESGVPSRFSGSGSGTDFTLTITSLQPDDFATYYCQQYDNLITFGQGTRLEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K145-H1 CAR
(Sequence ID No. 125)
DIQMTQSPSSLSASVGDRVSITCRASQDISRYLNWYQQKPGKAPKLLLYAASR
LESGVPSRFSGSGSGTDFTLTITSLQPDDFATYYCQQYNSYSRTFGQGTKVEIKGSTSGS
GKPGSGEGSTKGQVQLQESGPGLVKPSDTLSLTCLVSGGSISSNYWSWIRQAPGKGLE
WIGHVSYSGSTNYNPSLKSRVTISVDTSKNQFSLKLSSVTAADTAVYYCARESYYYYG
MDVWGQGTTVTVSSAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPR
K151-H1 CAR
(Sequence ID No. 126)
DIQMTQSPSSLSASVGDRVTITCRASQSISSYLNWYQQKPGKAPKLLIYAASSL
QSGVPSRFSGSGSGTDFTLTITSLQPDDFATYYCQQYDNLITFGQGTRLEIKGSTSGSGK
PGSGEGSTKGQVQLQESGPGLVKPSDTLSLTCLVSGGSISSNYWSWIRQAPGKGLEWIG
HVSYSGSTNYNPSLKSRVTISVDTSKNQFSLKLSSVTAADTAVYYCARESYYYYGMDV
WGQGTTVTVSSAAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K160-H1 CAR
(Sequence ID No. 127)
EIVLTQSPATLSVSPGERATLSCRASQSVSSNLAWYQQKPGQAPRLLIYGASTRATG
IPARFSGSGSGTEFTLTISRLEPEDFATYYCQQYNSYSRTFGQGTKVEIKGSTSGSGK
PGSGEGSTKGQVQLQESGPGLVKPSDTLSLTCLVSGGSISSNYWSWIRQAPGKGLEWIG
HVSYSGSTNYNPSLKSRVTISVDTSKNQFSLKLSSVTAADTAVYYCARESYYYYGMDV
WGQGTTVTVSSAAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
K173-H1 CAR
(Sequence ID No. 128)
DIQMTQSPSSLSASVGDRVTITCRASQSISSYLNWYQQKAGKAPKLLIYAASSLQS
GVPSRFSGSGSGTDFTLTISCLQSEDVATYYCQQYESYSRTFGQGTKVEIKGSTSGSG
KPGSGEGSTKGQVQLQESGPGLVKPSDTLSLTCLVSGGSISSNYWSWIRQAPGKGLEWI
GHVSYSGSTNYNPSLKSRVTISVDTSKNQFSLKLSSVTAADTAVYYCARESYYYYGMD
VWGQGTTVTVSSAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQ
ALPPR
L1-3M4E5H CAR
(Sequence ID No. 129)
QSALTQPPSASGSPGQSVTISCTGTSSDVGGYDFVSWYQQHPGEAPKLLVYDV
NNRPSGVSNRFSGSKSGNTASLTISGLQAEDEGDYYCSSYAGSNSVFGTGTKVTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSSAAAIEVMYPPPYLDNEKSNGTIIHVKGKHLCPSPLFPGP
SKPFWVLVVVGGVLACYSLLVTVAFIIFWVRSKRSRLLHSDYMNMTPRRPGPTRKHYQ
PYAPPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDT
YDALHMQALPPR
3M4E5H-L1 CAR
(Sequence ID No. 130)
EVQLLESGGGLVQPGGSLRLSCAASGFTFSTYQMSWVRQAPGKGLEWVSGIV
SSGGSTAYADSVKGRFTISRDNSKNTLYLQMNSLRAEDTAVYYCAGELLPYYGMDVW
GQGTTVTVSSEVQLLESGGGLVQPGGSLRLSCAASGFTFSTYQMSWVRQAPGKGLEW
VSGIVSSGGSTAYADSVKGRFTISRDNSKNTLYLQMNSLRAEDTAVYYCAGELLPYYG
MDVWGQGTTVTVSSQSALTQPPSASGSPGQSVTISCTGTSSDVGGYDFVSWYQQHPG
EAPKLLVYDVNNRPSGVSNRFSGSKSGNTASLTISGLQAEDEGDYYCSSYAGSNSVFGT
GTKVTVLAAAIEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPYAPPRDFAAYRS
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
L66-3M4E5H CAR
(Sequence ID No. 131)
QSALTQPASVSGSPGQSITISCTGTSSDVGGYEFVSWYQQHPGSAPKLIIYDVIE
RPFGVSYRFSASKSGNTASLTISGLQGEDEADYFCSSYTSSSTYVFGTGTKVTVLGSTSG
SGKPGSGEGSTKGEVQLLESGGGLVQPGGSLRLSCAASGFTFSTYQMSWVRQAPGKG
LEWVSGIVSSGGSTAYADSVKGRFTISRDNSKNTLYLQMNSLRAEDTAVYYCAGELLP
YYGMDVWGQGTTVTVSSAAAIEVMYPPPYLDNEKSNGTIIHVKGKHLCPSPLFPGPSK
PFWVLVVVGGVLACYSLLVTVAFIIFWVRSKRSRLLHSDYMNMTPRRPGPTRKHYQPY
APPRDFAAYRSRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYD
ALHMQALPPR
L73-3M4E5H CAR
(Sequence ID No. 132)
QSALTQPASVSGSPGQSITISCTGTGSDVGAYDYVSWYQHHPGRAPRLIIRDVS
VRPSGVPDRFSGSKSGNTASLTISGLQAEDEADYYCSSYSGSSTWVFGGGTKLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSSAAAIEVMYPPPYLDNEKSNGTIIHVKGKHLCPSPLFPGP
SKPFWVLVVVGGVLACYSLLVTVAFIIFWVRSKRSRLLHSDYMNMTPRRPGPTRKHYQ
PYAPPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDT
YDALHMQALPPR
L80-3M4E5H CAR
(Sequence ID No. 133)
QSALTQPASVSGSPGQSITISCTGTSSDVGSYNLVSWYQQHPGKAPKLMIYDVS
NRPSGVSYRFSGSKSGNTASLTISGLQAEDEADYYCSSYTSSSTFAVFGGGTQLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSSAAAIEVMYPPPYLDNEKSNGTIIHVKGKHLCPSPLFPGP
SKPFWVLVVVGGVLACYSLLVTVAFIIFWVRSKRSRLLHSDYMNMTPRRPGPTRKHYQ
PYAPPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDT
YDALHMQALPPR
L88-3M4E5H CAR
(Sequence ID No. 134)
QSALPQPASVSGSPGQSVTISCTGTSSDVGGYNYVSWYQQHPGKAPKLMIYD
VSKRPSGVPDRFSGSKSGNTASLTVSGLQAEDEADYFCCSYAGGYYVFGTGTKLTVLG
STSGSGKPGSGEGSTKGEVQLLESGGGLVQPGGSLRLSCAASGFTFSTYQMSWVRQAP
GKGLEWVSGIVSSGGSTAYADSVKGRFTISRDNSKNTLYLQMNSLRAEDTAVYYCAGE
LLPYYGMDVWGQGTTVTVSSAAAIEVMYPPPYLDNEKSNGTIIHVKGKHLCPSPLFPG
PSKPFWVLVVVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDT
YDALHMQALPPR
L102-3M4E5H CAR
(Sequence ID No. 135)
QSALTQPPSASGSPGQSVTISCTGTSSDVGGYNYVSWYQQHPGKAPKLMIYD
VSKRPSGVPDRFSGSKSGNTASLTISGLQTEDEADYYCSSYAGSGSTPFVFGTGTKLTVL
GSTSGSGKPGSGEGSTKGEVQLLESGGGLVQPGGSLRLSCAASGFTFSTYQMSWVRQA
PGKGLEWVSGIVSSGGSTAYADSVKGRFTISRDNSKNTLYLQMNSLRAEDTAVYYCAG
ELLPYYGMDVWGQGTTVTVSSAAAIEVMYPPPYLDNEKSNGTIIHVKGKHLCPSPLFP
GPSKPFWVLVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATK
DTYDALHMQALPPR
L124-3M4E5H CAR
(Sequence ID No. 136)
QSALTQPASVSGSPGQSITISCTGTSSDVGGYNYVSWYQQHPGKAPKLMIYDV
SNRPSRVPDRFAGSKSGNTASLTISGLQAEDEADYYCCSYAGRRYVFGTGTKLTVLGST
SGSGKPGSGEGSTKGEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGEL
LPYYGMDVWGQGTTVTVSSAAAIEVMYPPPYLDNEKSNGTIIHVKGKHLCPSPLFPGP
SKPFWVLVVVGGVLACYSLLVTVAFIIFWVRSKRSRLLHSDYMNMTPRRPGPTRKHYQ
PYAPPRDFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDT
YDALHMQALPPR
H73-3M4E5L CAR
(Sequence ID No. 137)
QLQLQESGPGLVKPSQTLSLTCTVSGGSISSYYWSWIRQPPGKGLEWIGEINHS
GSTNYNPSLKSRVTISVDTSKNQFSLKLSSVTAADTAVYYCARCPIYYYGMDVWGQGT
TVTVSSGSTSGSGKPGSGEGSTKGQSELTQPRSVSGSPGQSVTISCTGTSRDVGGYNYV
SWYQQHPGKAPKLIIHDVIERSSGVPDRFSGSKSGNTASLTISGLQAEDEADYYCWSFA
GSYYVFGTGTDVTVLAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPR

In the polypeptide of (C1) above, each of the amino acid sequences of Sequence ID Nos. 118 to 137 is an amino acid sequence that includes a combination of the heavy-chain variable region and the light-chain variable region that corresponds to any one of Combinations (1) to (18) for the first antibody of the present invention or the like, for example. In the polypeptides of (C2) and (C3) above, the amino acid sequences that correspond to the CDRH1, the CDRH2, the CDRH3, the CDRL1, the CDRL2, and the CDRL3 are conserved in the amino acid sequences of Sequence ID Nos. 118 to 137, for example. Regarding the amino acid sequences of the CDRs in the amino acid sequences of Sequence ID Nos. 118 to 137, the amino acid sequences that include the combinations of the heavy-chain variable region and the light-chain variable region that correspond to Combinations (1) to (18) for the first antibody of the present invention or the like can be referred to, for example.

The “identity” as used for the polypeptide of (C2) above refers to 80% or more, 85% or more, 90% or more, 95% or more, 96% or more, 97% or more, 98% or more, or 99% or more identity, for example.

The term “one or several” as used for the polypeptide of (C3) above regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

There is no particular limitation on a method for manufacturing the first CAR of the present invention, and the first CAR can be manufactured using genetic engineering techniques based on the amino acid sequence information described above, for example. Specifically, the first CAR can be manufactured as follows, for example. It should be noted that the present invention is not limited to this example.

First, an expression vector that includes a nucleic acid coding for the first CAR of the present invention is introduced into a host to obtain a transformant. Then, the transformant is cultured, and thus a first CAR-expressing transformant is obtained.

An example of the expression vector is a vector that includes a nucleic acid coding for the first CAR, and the descriptions of the above-described expression vector can be applied, for example. There is no particular limitation on the host, and the descriptions of the above-described host can be applied, for example.

### Second Chimeric Antigen Receptor

As described above, the second chimeric antigen receptor (CAR) of the present invention includes an antigen-binding domain, a transmembrane domain, and an intracellular signaling domain, and the antigen-binding domain includes the antibody of the present invention or the antigen-binding fragment thereof. The second CAR of the present invention is characterized in that the antigen-binding domain includes the second antibody of the present invention or the like, and there is no particular limitation on the other configurations and conditions. The descriptions of the CAR library, the first screening method, the first antibody or the like, the second antibody or the like, the gene, the expression vector, and the transformant of the present invention can be applied to the second CAR of the present invention. The second CAR of the present invention can be favorably used as a CAR for CAR-T cells against CD19-expressing cancer cells, for example.

The antigen-binding domain may be the second antibody of the present invention or the antigen-binding fragment of the second antibody of the present invention, for example. The antigen-binding fragment is preferably a single-chain antibody (scFv). If the antigen-binding domain is an scFv, the heavy-chain variable region and the light-chain variable region are arranged in this order from the C terminus or N terminus in the antigen-binding domain.

A specific example of the antigen-binding domain is a polypeptide of (bd) below.
(bd) A polypeptide of (bd1), (bd2), or (bd3) below:
   (bd1) a polypeptide consisting of the amino acid sequence of any one of Sequence ID Nos. 272 to 284,
   (bd2) a polypeptide consisting of an amino acid sequence having 80% or more identity to the amino acid sequence of any one of Sequence ID Nos. 272 to 284, the polypeptide being capable of binding to CD19, and
   (bd3) a polypeptide consisting of an amino acid sequence consisting of the amino acid sequence of any one of Sequence ID Nos. 272 to 284 with deletion, substitution, insertion, and/or addition of one or several amino acids, the polypeptide being capable of binding to CD19.

In the polypeptide of (bd1) above, each of the amino acid sequences of Sequence ID Nos. 272 to 284 is an amino acid sequence that includes a combination of the heavy-chain variable region and the light-chain variable region that corresponds to any one of Combinations (LA) to (LM) for the second antibody of the present invention or the like, for example. Regarding the amino acid sequences of the heavy-chain variable region and the light-chain variable region in each of the amino acid sequences of Sequence ID Nos. 272 to 284, the amino acid sequences that include the combinations of the heavy-chain variable region and the light-chain variable region that correspond to Combinations (LA) to (LM) for the second antibody of the present invention or the like can be referred to, for example. In the polypeptides of (bd2) and (bd3) above, the amino acid sequences that correspond to the CDRH1, the CDRH2, the CDRH3, the CDRL1, the CDRL2, and the CDRL3 are conserved in the amino acid sequences of Sequence ID Nos. 272 to 284, for example. Regarding the amino acid sequences of the CDRs in the amino acid sequences of Sequence ID Nos. 272 to 284, the amino acid sequences of the CDRs included in the amino acid sequences that include the combinations of the heavy-chain variable region and the light-chain variable region that correspond to Combinations (LA) to (LM) for the second antibody of the present invention or the like can be referred to, for example.

Antigen-Binding Domain of L4-18H
(Sequence ID No. 272)
SYELTQPPSVSVSPGQTASITCSGDKLGDKYACWYQQKPGQSPVLVIYQDSKR
PSGIPERFSGSNSGNTATLTISGTQAMDEADYYCQAWDSSTHVVFGGGTKLTVLGSTSG
SGKPGSGEGSTKGEVQLVESGGGLVQPGRSLRLSCAASGFTFDDYAMHWVRQAPGKG
LEWVSGISWNSGRIGYADSVKGRFTISRDNAKNSLFLQMNSLRAEDTAVYYCARDQG
YHYYDSAEHAFDIWGQGTVVTVSS
Antigen-Binding Domain of L7-18H
(Sequence ID No. 273)
QSALTQPRSVSGSPGQSVTISCTGTSSDVGGYNYVSWYQQHPGKAPKLMIYD
VSNRPSGVSNRFSGSKSGNTASLTISGLQTGDEADYYCGTWDTSLTAVVFGGGTELTVL
GSTSGSGKPGSGEGSTKGEVQLVESGGGLVQPGRSLRLSCAASGFTFDDYAMHWVRQ
APGKGLEWVSGISWNSGRIGYADSVKGRFTISRDNAKNSLFLQMNSLRAEDTAVYYCA
RDQGYHYYDSAEHAFDIWGQGTVVTVSS
Antigen-Binding Domain of L9-18H
(Sequence ID No. 274)
QSVLTQPPSVSAAPGQKVTISCSGSWSNIGDDHVSWYQQFPGAAPKLLIYDTS
KRPSRVADRFSGSKSGASATLAITGLQAGDEADYYCGTWESSLSGVVFGGGTELTVLG
STSGSGKPGSGEGSTKGEVQLVESGGGLVQPGRSLRLSCAASGFTFDDYAMHWVRQA
PGKGLEWVSGISWNSGRIGYADSVKGRFTISRDNAKNSLFLQMNSLRAEDTAVYYCAR
DQGYHYYDSAEHAFDIWGQGTVVTVSS
Antigen-Binding Domain of L13-18H
(Sequence ID No. 275)
QSALTQPASVSGSPGQSITISCTGLSSDVGGYDYVSWYQQHPGIAPKLMIYDVT
NRPSGVSSRFSGSKSGNTASLTISGLQAEDEADYYCSSYTTSTTWVFGAGTKLTVLGST
SGSGKPGSGEGSTKGEVQLVESGGGLVQPGRSLRLSCAASGFTFDDYAMHWVRQAPG
KGLEWVSGISWNSGRIGYADSVKGRFTISRDNAKNSLFLQMNSLRAEDTAVYYCARD
QGYHYYDSAEHAFDIWGQGTVVTVSS
Antigen-Binding Domain of L14-18H
(Sequence ID No. 276)
QSALTQPASVSGSPGQSITISCTGTTSDVGTTNYVSWYQQHPGKAPKLLIYDVT
NRPSGVPDRFSGSKSANTASLTISGLQAEDEADYYCCSYAGSYTFVVFGGGTELTVLGS
TSGSGKPGSGEGSTKGEVQLVESGGGLVQPGRSLRLSCAASGFTFDDYAMHWVRQAP
GKGLEWVSGISWNSGRIGYADSVKGRFTISRDNAKNSLFLQMNSLRAEDTAVYYCAR
DQGYHYYDSAEHAFDIWGQGTVVTVSS
Antigen-Binding Domain of L16-18H
(Sequence ID No. 277)
QSALTQPPSASGSPGQSVTISCTGTSSDVGVYNYVSWYQQHPGKAPKLMIYD
VSKRPSGVPDRFSGSKSANTASLTISGLQAEDEADYYCAAWDDSLNGVVFGGGTQLTV
LGSTSGSGKPGSGEGSTKGEVQLVESGGGLVQPGRSLRLSCAASGFTFDDYAMHWVR
QAPGKGLEWVSGISWNSGRIGYADSVKGRFTISRDNAKNSLFLQMNSLRAEDTAVYYC
ARDQGYHYYDSAEHAFDIWGQGTVVTVSS
Antigen-Binding Domain of L17-18H
(Sequence ID No. 278)
QSVLTQPPSASGTPGQRVTISCSGSSSNIGNNYVCWYQHLPGTAPKLLIYDNVK
RPSGIPDRFSGSKSGTSASLAISGLRSEDEADYYCAAWDDSLSAIFGGGTELTVLGSTSG
SGKPGSGEGSTKGEVQLVESGGGLVQPGRSLRLSCAASGFTFDDYAMHWVRQAPGKG
LEWVSGISWNSGRIGYADSVKGRFTISRDNAKNSLFLQMNSLRAEDTAVYYCARDQG
YHYYDSAEHAFDIWGQGTVVTVSS
Antigen-Binding Domain of L22-18H
(Sequence ID No. 279)
QSALTQPASVSGSPGQSITISCTGTSSDVGGYNYVSWYQQHPGKAPKLMIYDV
SNRPSGVPDRFSGSKSGTSASLAISGLQSEDEADYYCHSYDSSLSHVFGTGTKVTVLGS
TSGSGKPGSGEGSTKGEVQLVESGGGLVQPGRSLRLSCAASGFTFDDYAMHWVRQAP
GKGLEWVSGISWNSGRIGYADSVKGRFTISRDNAKNSLFLQMNSLRAEDTAVYYCAR
DQGYHYYDSAEHAFDIWGQGTVVTVSS
Antigen-Binding Domain of K4-18H
(Sequence ID No. 280)
EIVLTQSPGTLSLSPGERATLSCRASQSVSSNLAWYQQKPGQAPRLLIYGASTR
ATGIPARFSGSGSGTEFTLTISSLQSEDFAVYYCQQYNNWPPLYTFGQGTKLEIKGSTSGS
GKPGSGEGSTKGEVQLVESGGGLVQPGRSLRLSCAASGFTFDDYAMHWVRQAPGKGL
EWVSGISWNSGRIGYADSVKGRFTISRDNAKNSLFLQMNSLRAEDTAVYYCARDQGY
HYYDSAEHAFDIWGQGTVVTVSS
Antigen-Binding Domain of K5-18H
(Sequence ID No. 281)
EIVLTQSPGTLSLSPGERATLSCRASQSVSSYLAWYQQKPGQAPRLLIYDASNR
ATGIPARFSGSGSGTDFTLTISSLEPEDFATYYCQQSYSTLLYTFGQGTKLEIKGSTSGSG
KPGSGEGSTKGEVQLVESGGGLVQPGRSLRLSCAASGFTFDDYAMHWVRQAPGKGLE
WVSGISWNSGRIGYADSVKGRFTISRDNAKNSLFLQMNSLRAEDTAVYYCARDQGYH
YYDSAEHAFDIWGQGTVVTVSS
Antigen-Binding Domain of K6-18H
(Sequence ID No. 282)
EIVLTRSPATLSLSPGERATLSCRASQSVSSYLAWYQQKPGQAPRLLIYDASNR
ATGISARFSGSGSGTEFTLTISSLQSEDIATYYCQQYDSLPLTFGGGTKLEIKGSTSGSGKP
GSGEGSTKGEVQLVESGGGLVQPGRSLRLSCAASGFTFDDYAMHWVRQAPGKGLEW
VSGISWNSGRIGYADSVKGRFTISRDNAKNSLFLQMNSLRAEDTAVYYCARDQGYHY
YDSAEHAFDIWGQGTVVTVSS
Antigen-Binding Domain of K9-18H
(Sequence ID No. 283)
EIVLTQSPATLSLSPGERATVSCRPSQTISASSVAWYQQKAGQAPRLLIYGASSR
ATGIPDRFSGSGSGTDFTLTISRLEPEDFAVYYCQQFNEWPLTFGGGTKVEIKGSTSGSG
KPGSGEGSTKGEVQLVESGGGLVQPGRSLRLSCAASGFTFDDYAMHWVRQAPGKGLE
WVSGISWNSGRIGYADSVKGRFTISRDNAKNSLFLQMNSLRAEDTAVYYCARDQGYH
YYDSAEHAFDIWGQGTVVTVSS
Antigen-Binding Domain of K10-18H
(Sequence ID No. 284)
EIVLTQSPATLSLSPGERATLSCRASQSVSSNLAWYQQKLGQAPRLLIYGASSR
ATGIPDRFSGSGSGTDFTLTISRLEPEDFAVYYCQQYGSSPDIFTFGPGTKVDIKGSTSGS
GKPGSGEGSTKGEVQLVESGGGLVQPGRSLRLSCAASGFTFDDYAMHWVRQAPGKGL
EWVSGISWNSGRIGYADSVKGRFTISRDNAKNSLFLQMNSLRAEDTAVYYCARDQGY
HYYDSAEHAFDIWGQGTVVTVSS

In the polypeptide of (bd1) above, the amino acid sequences of the heavy-chain variable region and the light-chain variable region may also be arranged in the reverse order. In a specific example, the polypeptide of (bd1) above may be a polypeptide obtained by exchanging the amino acid sequences of the heavy-chain variable region and the light-chain variable region in the amino acid sequences of Sequence ID Nos. 272 to 284.

The “identity” as used for the polypeptide of (bd2) above refers to 80% or more, 85% or more, 90% or more, 95% or more, 96% or more, 97% or more, 98% or more, or 99% or more identity, for example.

The term “one or several” as used for the polypeptide of (bd3) above regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

The descriptions of the first transmembrane domain and the first intracellular signaling domain of the CAR library of the present invention can be respectively applied to the above-mentioned transmembrane domain and intracellular signaling domain, for example.

A specific example of the second CAR of the present invention is a polypeptide of (CA) below, for example.

(CA) A polypeptide of (CA1), (CA2), or (CA3) below:
(CA1) a polypeptide consisting of the amino acid sequence of any one of Sequence ID Nos. 285 to 297,
(CA2) a polypeptide consisting of an amino acid sequence having 80% or more identity to the amino acid sequence of any one of Sequence ID Nos. 285 to 297, the polypeptide being capable of binding to CD19, and
(CA3) a polypeptide consisting of an amino acid sequence consisting of the amino acid sequence of any one of Sequence ID Nos. 285 to 297 with deletion, substitution, insertion, and/or addition of one or several amino acids, the polypeptide being capable of binding to CD19.

L4-18H CAR
(Sequence ID No. 285)
SYELTQPPSVSVSPGQTASITCSGDKLGDKYACWYQQKPGQSPVL
VIYQDSKRPSGIPERFSGSNSGNTATLTISGTQAMDEADYYCQAW
DSSTHVVFGGGTKLTVLGSTSGSGKPGSGEGSTKGEVQLVESGGG
LVQPGRSLRLSCAASGFTFDDYAMHWVRQAPGKGLEWVSGISWNS
GRIGYADSVKGRFTISRDNAKNSLFLQMNSLRAEDTAVYYCARDQ
GYHYYDSAEHAFDIWGQGTVVTVSSAAAIEVMYPPPYLDNEKSNG
TIIHVKGKHLCPSPLFPGPSKPFWVLVVVGGVLACYSLLVTVAFI
IFWVRSKRSRLLHSDYMNMTPRRPGPTRKHYQPYAPPRDFAAYRS
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR
L7-18H CAR
(Sequence ID No. 286)
QSALTQPRSVSGSPGQSVTISCTGTSSDVGGYNYVSWYQQHPGKA
PKLMIYDVSNRPSGVSNRFSGSKSGNTASLTISGLQTGDEADYYC
GTWDTSLTAVVFGGGTELTVLGSTSGSGKPGSGEGSTKGEVQLVE
SGGGLVQPGRSLRLSCAASGFTFDDYAMHWVRQAPGKGLEWVSGI
SWNSGRIGYADSVKGRFTISRDNAKNSLFLQMNSLRAEDTAVYYC
ARDQGYHYYDSAEHAFDIWGQGTVVTVSSAAAIEVMYPPPYLDNE
KSNGTIIHVKGKHLCPSPLFPGPSKPFWVLVVVGGVLACYSLLVT
VAFIIFWVRSKRSRLLHSDYMNMTPRRPGPTRKHYQPYAPPRDFA
AYRSRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
L9-18H CAR
(Sequence ID No. 287)
QSVLTQPPSVSAAPGQKVTISCSGSWSNIGDDHVSWYQQFPGAAP
KLLIYDTSKRPSRVADRFSGSKSGASATLAITGLQAGDEADYYCG
TWESSLSGVVFGGGTELTVLGSTSGSGKPGSGEGSTKGEVQLVES
GGGLVQPGRSLRLSCAASGFTFDDYAMHWVRQAPGKGLEWVSGIS
WNSGRIGYADSVKGRFTISRDNAKNSLFLQMNSLRAEDTAVYYCA
RDQGYHYYDSAEHAFDIWGQGTVVTVSSAAAIEVMYPPPYLDNEK
SNGTIIHVKGKHLCPSPLFPGPSKPFWVLVVVGGVLACYSLLVTV
AFIIFWVRSKRSRLLHSDYMNMTPRRPGPTRKHYQPYAPPRDFAA
YRSRVKFSRSADAPAYQQGQNQLYNELNLGRREEYDVLDKRRGRD
PEMGGKPRRKNPQEGLYNELQKDKMAEAYSEIGMKGERRRGKGHD
GLYQGLSTATKDTYDALHMQALPPR
L13-18H CAR
(Sequence ID No. 288)
QSALTQPASVSGSPGQSITISCTGLSSDVGGYDYVSWYQQHPGIA
PKLMIYDVTNRPSGVSSRFSGSKSGNTASLTISGLQAEDEADYYC
SSYTTSTTWVFGAGTKLTVLGSTSGSGKPGSGEGSTKGEVQLVES
GGGLVQPGRSLRLSCAASGFTFDDYAMHWVRQAPGKGLEWVSGIS
WNSGRIGYADSVKGRFTISRDNAKNSLFLQMNSLRAEDTAVYYCA
RDQGYHYYDSAEHAFDIWGQGTVVTVSSAAAIEVMYPPPYLDNEK
SNGTIIHVKGKHLCPSPLFPGPSKPFWVLVVVGGVLACYSLLVTV
AFIIFWVRSKRSRLLHSDYMNMTPRRPGPTRKHYQPYAPPRDFAA
YRSRVKFSRSADAPAYQQGQNQLYNELNLGRREEYDVLDKRRGRD
PEMGGKPRRKNPQEGLYNELQKDKMAEAYSEIGMKGERRRGKGHD
GLYQGLSTATKDTYDALHMQALPPR
L14-18H CAR
(Sequence ID No. 289)
QSALTQPASVSGSPGQSITISCTGTTSDVGTTNYVSWYQQHPGKA
PKLLIYDVTNRPSGVPDRFSGSKSANTASLTISGLQAEDEADYYC
CSYAGSYTFVVFGGGTELTVLGSTSGSGKPGSGEGSTKGEVQLVE
SGGGLVQPGRSLRLSCAASGFTFDDYAMHWVRQAPGKGLEWVSGI
SWNSGRIGYADSVKGRFTISRDNAKNSLFLQMNSLRAEDTAVYYC
ARDQGYHYYDSAEHAFDIWGQGTVVTVSSAAAIEVMYPPPYLDNE
KSNGTIIHVKGKHLCPSPLFPGPSKPFWVLVVVGGVLACYSLLVT
VAFIIFWVRSKRSRLLHSDYMNMTPRRPGPTRKHYQPYAPPRDFA
AYRSRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
L16-18H CAR
(Sequence ID No. 290)
QSALTQPPSASGSPGQSVTISCTGTSSDVGVYNYVSWYQQHPGKA
PKLMIYDVSKRPSGVPDRFSGSKSANTASLTISGLQAEDEADYYC
AAWDDSLNGVVFGGGTQLTVLGSTSGSGKPGSGEGSTKGEVQLVE
SGGGLVQPGRSLRLSCAASGFTFDDYAMHWVRQAPGKGLEWVSGI
SWNSGRIGYADSVKGRFTISRDNAKNSLFLQMNSLRAEDTAVYYC
ARDQGYHYYDSAEHAFDIWGQGTVVTVSSAAAIEVMYPPPYLDNE
KSNGTIIHVKGKHLCPSPLFPGPSKPFWVLVVVGGVLACYSLLVT
VAFIIFWVRSKRSRLLHSDYMNMTPRRPGPTRKHYQPYAPPRDFA
AYRSRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
L17-18H CAR
(Sequence ID No. 291)
QSVLTQPPSASGTPGQRVTISCSGSSSNIGNNYVCWYQHLPGTAP
KLLIYDNVKRPSGIPDRFSGSKSGTSASLAISGLRSEDEADYYCA
AWDDSLSAIFGGGTELTVLGSTSGSGKPGSGEGSTKGEVQLVESG
GGLVQPGRSLRLSCAASGFTFDDYAMHWVRQAPGKGLEWVSGISW
NSGRIGYADSVKGRFTISRDNAKNSLFLQMNSLRAEDTAVYYCAR
DQGYHYYDSAEHAFDIWGQGTVVTVSSAAAIEVMYPPPYLDNEKS
NGTIIHVKGKHLCPSPLFPGPSKPFWVLVVVGGVLACYSLLVTVA
FIIFWVRSKRSRLLHSDYMNMTPRRPGPTRKHYQPYAPPRDFAAY
RSRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR
L22-18H CAR
(Sequence ID No. 292)
QSALTQPASVSGSPGQSITISCTGTSSDVGGYNYVSWYQQHPGKA
PKLMIYDVSNRPSGVPDRFSGSKSGTSASLAISGLQSEDEADYYC
HSYDSSLSHVFGTGTKVTVLGSTSGSGKPGSGEGSTKGEVQLVES
GGGLVQPGRSLRLSCAASGFTFDDYAMHWVRQAPGKGLEWVSGIS
WNSGRIGYADSVKGRFTISRDNAKNSLFLQMNSLRAEDTAVYYCA
RDQGYHYYDSAEHAFDIWGQGTVVTVSSAAAIEVMYPPPYLDNEK
SNGTIIHVKGKHLCPSPLFPGPSKPFWVLVVVGGVLACYSLLVTV
AFIIFWVRSKRSRLLHSDYMNMTPRRPGPTRKHYQPYAPPRDFAA
YRSRVKFSRSADAPAYQQGQNQLYNELNLGRREEYDVLDKRRGRD
PEMGGKPRRKNPQEGLYNELQKDKMAEAYSEIGMKGERRRGKGHD
GLYQGLSTATKDTYDALHMQALPPR
K4-18H CAR
(Sequence ID No. 293)
EIVLTQSPGTLSLSPGERATLSCRASQSVSSNLAWYQQKPGQAPR
LLIYGASTRATGIPARFSGSGSGTEFTLTISSLQSEDFAVYYCQQ
YNNWPPLYTFGQGTKLEIKGSTSGSGKPGSGEGSTKGEVQLVESG
GGLVQPGRSLRLSCAASGFTFDDYAMHWVRQAPGKGLEWVSGISW
NSGRIGYADSVKGRFTISRDNAKNSLFLQMNSLRAEDTAVYYCAR
DQGYHYYDSAEHAFDIWGQGTVVTVSSAAAIEVMYPPPYLDNEKS
NGTIIHVKGKHLCPSPLFPGPSKPFWVLVVVGGVLACYSLLVTVA
FIIFWVRSKRSRLLHSDYMNMTPRRPGPTRKHYQPYAPPRDFAAY
RSRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR
K5-18H CAR
(Sequence ID No. 294)
EIVLTQSPGTLSLSPGERATLSCRASQSVSSYLAWYQQKPGQAPR
LLIYDASNRATGIPARFSGSGSGTDFTLTISSLEPEDFATYYCQQ
SYSTLLYTFGQGTKLEIKGSTSGSGKPGSGEGSTKGEVQLVESGG
GLVQPGRSLRLSCAASGFTFDDYAMHWVRQAPGKGLEWVSGISWN
SGRIGYADSVKGRFTISRDNAKNSLFLQMNSLRAEDTAVYYCARD
QGYHYYDSAEHAFDIWGQGTVVTVSSAAAIEVMYPPPYLDNEKSN
GTIIHVKGKHLCPSPLFPGPSKPFWVLVVVGGVLACYSLLVTVAF
IIFWVRSKRSRLLHSDYMNMTPRRPGPTRKHYQPYAPPRDFAAYR
SRVKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPR
K6-18H CAR
(Sequence ID No. 295)
EIVLTRSPATLSLSPGERATLSCRASQSVSSYLAWYQQKPGQAPR
LLIYDASNRATGISARFSGSGSGTEFTLTISSLQSEDIATYYCQQ
YDSLPLTFGGGTKLEIKGSTSGSGKPGSGEGSTKGEVQLVESGGG
LVQPGRSLRLSCAASGFTFDDYAMHWVRQAPGKGLEWVSGISWNS
GRIGYADSVKGRFTISRDNAKNSLFLQMNSLRAEDTAVYYCARDQ
GYHYYDSAEHAFDIWGQGTVVTVSSAAAIEVMYPPPYLDNEKSNG
TIIHVKGKHLCPSPLFPGPSKPFWVLVVVGGVLACYSLLVTVAFI
IFWVRSKRSRLLHSDYMNMTPRRPGPTRKHYQPYAPPRDFAAYRS
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR
K9-18H CAR
(Sequence ID No. 296)
EIVLTQSPATLSLSPGERATVSCRPSQTISASSVAWYQQKAGQAP
RLLIYGASSRATGIPDRFSGSGSGTDFTLTISRLEPEDFAVYYCQ
QFNEWPLTFGGGTKVEIKGSTSGSGKPGSGEGSTKGEVQLVESGG
GLVQPGRSLRLSCAASGFTFDDYAMHWVRQAPGKGLEWVSGISWN
SGRIGYADSVKGRFTISRDNAKNSLFLQMNSLRAEDTAVYYCARD
QGYHYYDSAEHAFDIWGQGTVVTVSSAAAIEVMYPPPYLDNEKSN
GTIIHVKGKHLCPSPLFPGPSKPFWVLVVVGGVLACYSLLVTVAF
IIFWVRSKRSRLLHSDYMNMTPRRPGPTRKHYQPYAPPRDFAAYR
SRVKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPR
K10-18H CAR
(Sequence ID No. 297)
EIVLTQSPATLSLSPGERATLSCRASQSVSSNLAWYQQKLGQAPR
LLIYGASSRATGIPDRFSGSGSGTDFTLTISRLEPEDFAVYYCQQ
YGSSPDIFTFGPGTKVDIKGSTSGSGKPGSGEGSTKGEVQLVESG
GGLVQPGRSLRLSCAASGFTFDDYAMHWVRQAPGKGLEWVSGISW
NSGRIGYADSVKGRFTISRDNAKNSLFLQMNSLRAEDTAVYYCAR
DQGYHYYDSAEHAFDIWGQGTVVTVSSAAAIEVMYPPPYLDNEKS
NGTIIHVKGKHLCPSPLFPGPSKPFWVLVVVGGVLACYSLLVTVA
FIIFWVRSKRSRLLHSDYMNMTPRRPGPTRKHYQPYAPPRDFAAY
RSRVKFSRSADAPAYQQGQNQLYNELNLGRREEYDVLDKRRGRDP
EMGGKPRRKNPQEGLYNELQKDKMAEAYSEIGMKGERRRGKGHDG
LYQGLSTATKDTYDALHMQALPPR

In the polypeptide of (CA1) above, each of the amino acid sequences of Sequence ID Nos. 285 to 297 is an amino acid sequence that includes a combination of the heavy-chain variable region and the light-chain variable region that corresponds to any one of Combinations (LA) to (LM) for the second antibody of the present invention or the like, for example. In the polypeptides of (CA2) and (CA3) above, the amino acid sequences that correspond to the CDRH1, the CDRH2, the CDRH3, the CDRL1, the CDRL2, and the CDRL3 are conserved in the amino acid sequences of Sequence ID Nos. 285 to 297, for example. Regarding the amino acid sequences of the CDRs in the amino acid sequences of Sequence ID Nos. 285 to 297, the amino acid sequences that include the combinations of the heavy-chain variable region and the light-chain variable region that correspond to Combinations (LA) to (LM) for the second antibody of the present invention or the like can be referred to, for example.

The “identity” as used for the polypeptide of (CA2) above refers to 80% or more, 85% or more, 90% or more, 95% or more, 96% or more, 97% or more, 98% or more, or 99% or more identity, for example.

The term “one or several” as used for the polypeptide of (CA3) above regarding substitution and the like refers to 1 to 20, 1 to 15, 1 to 10, 1 to 9, 1 to 8, 1 to 7, 1 to 6, 1 to 5, 1 to 4, 1 to 3, 1 or 2, or 1, for example.

There is no particular limitation on a method for manufacturing the second CAR of the present invention, and the second CAR can be manufactured using genetic engineering techniques based on the amino acid sequence information described above, for example. Specifically, the second CAR can be manufactured as follows, for example. It should be noted that the present invention is not limited to this example.

First, an expression vector that includes a nucleic acid coding for the second CAR of the present invention is introduced into a host to obtain a transformant. Then, the transformant is cultured, and thus a second CAR-expressing transformant is obtained.

An example of the expression vector is a vector that includes a nucleic acid coding for the second CAR, and the descriptions of the above-described expression vector can be applied, for example. There is no particular limitation on the host, and the descriptions of the above-described host can be applied, for example.

### Nucleic Acid

As described above, the nucleic acid of the present invention codes for the chimeric antigen receptor of the present invention. The nucleic acid of the present invention is characterized by coding for the first chimeric antigen receptor or second chimeric antigen receptor of the present invention (also collectively referred to as the “chimeric antigen receptor of the present invention” hereinafter), and there is no particular limitation on the other configurations and conditions. With the nucleic acid of the present invention, the CAR of the present invention can be expressed in cells, for example. The descriptions of the CAR library, the first screening method, the first antibody or the like, the first antibody or the like, the gene, the expression vector, the transformant, the first chimeric antigen receptor, and the second chimeric antigen receptor of the present invention can be applied to the nucleic acid of the present invention, for example.

The nucleic acid of the present invention can be prepared based on the amino acid sequence of the CAR of the present invention using an ordinary method, for example. In a specific example, the nucleic acid coding for the CAR of the present invention can be prepared based on the base sequence coding for the amino acid sequence obtained from the database in which the amino acid sequences of the above-described domains are registered, using a molecular biological technique and/or a chemical synthesis method, for example. The base sequence of the nucleic acid may be subjected to codon optimization in accordance with the source of cells in which the CAR of the present invention is to be expressed, for example.

A specific example of the nucleic acid of the present invention is a polynucleotide of (c) below.

(c) A polynucleotide (nucleic acid) of (c1), (c2), (c3), (c4), (c5), or (c6) below:
(c1) a polynucleotide coding for a polypeptide consisting of the amino acid sequence of any one of Sequence ID Nos. 118 to 137,
(c2) a polynucleotide coding for a polypeptide consisting of an amino acid sequence having 80% or more identity to the amino acid sequence of any one of Sequence ID Nos. 118 to 137, the polypeptide being capable of binding to A2/NY-ESO-1₁₅₇,
(c3) a polynucleotide coding for a polypeptide consisting of an amino acid sequence consisting of the amino acid sequence of any one of Sequence ID Nos. 118 to 137 with deletion, substitution, insertion, and/or addition of one or several amino acids, the polypeptide being capable of binding to A2/NY-ESO-1₁₅₇,
(c4) a polynucleotide consisting of the base sequence of any one of Sequence ID Nos. 138 to 157,
(c5) a polynucleotide consisting of a base sequence having 80% or more identity to the base sequence of any one of Sequence ID Nos. 138 to 157, and coding for a polypeptide capable of binding to A2/NY-ESO-1₁₅₇, and
(c6) a polynucleotide consisting of a base sequence consisting of the base sequence of any one of Sequence ID Nos. 138 to 157 with deletion, substitution, insertion, and/or addition of one or several bases, and coding for a polypeptide capable of binding to A2/NY-ESO-1₁₅₇.

The polynucleotides of (c1) to (c3) above are nucleic acids coding for the polypeptides of (C1) to (C3) above, respectively, and the descriptions of the polypeptides of (C1) to (C3) above can be applied.

In the polynucleotide of (c4) above, each of the base sequences of Sequence ID Nos. 138 to 157 is a polynucleotide coding for a polypeptide that includes an amino acid sequence that includes a combination of the heavy-chain variable region and the light-chain variable region that corresponds to any one of Combinations (1) to (18) for the first antibody of the present invention or the like, for example. In the polynucleotides of (c5) and (c6) above, the polynucleotides coding for the amino acid sequences that correspond to the CDRH1, the CDRH2, the CDRH3, the CDRL1, the CDRL2, and the CDRL3 are conserved in the base sequences of Sequence ID Nos. 138 to 157, for example. Regarding the base sequences coding for the amino acid sequences of the CDRs in the base sequences of Sequence ID Nos. 138 to 157, the amino acid sequences of the CDRs included in the amino acid sequences that include the combinations of the heavy-chain variable region and the light-chain variable region that correspond to Combinations (1) to (18) for the first antibody of the present invention or the like can be referred to, for example.

The “identity” as used for the polynucleotide of (c5) above refers to 80% or more, 85% or more, 90% or more, 95% or more, 96% or more, 97% or more, 98% or more, or 99% or more identity, for example.

The term “one or several” as used for the polynucleotide of (c6) above regarding substitution and the like refers to 1 to 280, 1 to 210, 1 to 140, 1 to 70, 1 to 63, 1 to 56, 1 to 49, 1 to 35, 1 to 28, 1 to 21, 1 to 14, 1 to 7, 1 to 6, 1 to 5, 1 to 4, 1 to 3, 1 or 2, or 1, for example.

Polynucleotide Coding for H1-3M4E5L CAR
(Sequence ID No. 138)
5′-CAGGTGCAGCTGCAGGAGTCGGGCCCAGGACTGGTGAAGCCT
TCGGATACCCTGTCCCTCACCTGTCTTGTCTCTGGTGGCTCCATC
AGTAGTAATTACTGGAGCTGGATCCGGCAGGCCCCAGGGAAGGGA
CTGGAGTGGATTGGACATGTCTCCTACAGTGGGAGCACCAACTAC
AACCCCTCCCTCAAGAGTCGAGTTACCATATCAGTAGACACGTCT
AAGAACCAGTTCTCCCTGAAGCTGAGCTCTGTGACTGCCGCGGAC
ACGGCCGTGTATTACTGTGCGAGAGAGTCCTACTACTACTACGGT
ATGGACGTCTGGGGCCAAGGGACCACGGTCACCGTCTCCTCAGGC
TCTACAAGCGGCTCTGGCAAGCCTGGATCTGGCGAGGGAAGCACC
AAGGGCCAGAGCGAGCTGACACAGCCTAGATCCGTGTCTGGCAGC
CCTGGCCAGAGCGTGACCATCAGCTGTACCGGCACCAGCAGAGAT
GTGGGCGGCTACAACTACGTGTCCTGGTATCAGCAGCATCCCGGC
AAGGCCCCCAAGCTGATCATCCACGACGTGATCGAGCGGAGCAGC
GGCGTGCCCGATAGATTCAGCGGCAGCAAGAGCGGCAACACCGCC
AGCCTGACAATCAGCGGACTGCAGGCCGAGGACGAGGCCGACTAC
TACTGTTGGAGCTTCGCCGGCAGCTACTACGTGTTCGGCACCGGC
ACCGATGTGACCGTGCTGGCGGCCGCAATCGAAGTGATGTACCCC
CCTCCCTACCTGGACAACGAGAAGTCCAACGGCACCATTATCCAC
GTGAAGGGAAAGCACCTGTGCCCCAGCCCTCTGTTCCCTGGCCCT
AGCAAGCCTTTCTGGGTGCTGGTGGTCGTGGGCGGAGTGCTGGCC
TGTTATAGCCTGCTCGTGACCGTGGCCTTCATCATCTTTTGGGTG
CGCAGCAAGCGGAGCCGGCTGCTGCACAGCGACTACATGAACATG
ACCCCCAGACGGCCCGGACCCACCAGAAAGCACTACCAGCCTTAC
GCCCCTCCCAGAGACTTCGCCGCCTACAGATCTCGAGTGAAGTTC
AGCAGAAGCGCCGACGCCCCTGCCTATCAGCAGGGCCAGAACCAG
CTGTACAACGAGCTGAACCTGGGCAGACGGGAAGAGTACGACGTG
CTGGACAAGCGGAGAGGCAGGGACCCTGAGATGGGCGGCAAGCCC
AGAAGAAAGAACCCCCAGGAAGGCCTGTATAACGAACTGCAGAAA
GACAAGATGGCCGAGGCCTACAGCGAGATCGGCATGAAGGGCGAG
CGGAGAAGAGGCAAGGGCCACGATGGCCTGTACCAGGGCCTGAGC
ACCGCCACCAAGGACACCTATGACGCCCTGCACATGCAGGCCCTG
CCCCCCAGA-3′
Polynucleotide Coding for K52-H1 CAR
(Sequence ID No. 139)
5′-GACATCCAGATGACCCAGTCTCCATCTGCCATGTCTGCATCT
GTAGGAGACAGAGTCACCATCACTTGCCGGGCAAGTCAGAGCATT
AGCAGCTATTTAAATTGGTATCAGCAGAAACCAGGGAAAGCCCCT
AAGCTCCTGATCTATGCTGCATCCAGTTTGCAAAGTGGGGTCCCA
TCAAGGTTCAGTGGCAGTGGATCTGGGACAGATTTCACTCTCACC
ATCAGCAGTCTGCAGCCTGAAGATTTTGCAACTTACTATTGTCAG
CAATATTATAGTACTCCTCAAACTTTCGGCCCTGGGACCAAAGTG
GATATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K73-H1 CAR
(Sequence ID No. 140)
5′-GACATCCAGATGACCCAGTCTCCATCCTCCCTGTCTGCATCT
GTAGGAGACAGAGTCACCATCACTTGCCGGGCAAGTCAGAGCATT
AGCAGCTATTTAAATTGGTATCAGCAGAAAGCAGGGAAAGCCCCT
AAGCTTCTGATCTATGCTGCATCCAGTTTGCAAAGTGGGGTCCCA
TCAAGGTTCAGCGGCAGTGGATCTGGGACAGATTTCACTCTCACC
ATCAGCTGCCTGCAGTCTGAAGATGTTGCAACCTATTACTGCCAA
CAGTATGAAAGTTATCGAAGGTCGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K121-H1 CAR
(Sequence ID No. 141)
5′-GACATCCAGATGACCCAGTCTCCATCCTCCCTGTCTGCATCT
GTAGGAGACAGAGTCACCATCACTTGCCGGGCAAGTCAGAGCATT
AGCAGCTATTTAAATTGGTATCAGCAGAAACCAGGGAAAGCCCCT
AAGCTCCTGATCTATGCTGCATCCAGTTTGCAAAGTGGGGTCCCA
TCAAGGTTCAGTGGCAGTGGATCTGGGACAGATTTCACTCTCACC
ATCAGCTGCCTGCAGTCTGAAGATTTTGCAACTTATTACTGCCAA
CAGTATAATAGTTATTCCCGGACGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K124-H1 CAR
(Sequence ID No. 142)
5′-GACATCCAGATGACCCAGTCTCCATCCTCCCTGTCTGCATCT
GTAGGAGACAGAGTCACCATCACTTGCCGGGCAAGTCAGAGCATT
AGCAGCTATTTAAATTGGTATCAGCAGAAACCAGGGAAAGCCCCT
AAGCTCCTGATCTATGCTGCATCCAGATTGGAAAGTGGGGTCCCA
TCAAGGTTCAGTGGCAGTGGATCTGGGACAGATTTCACTCTCACC
ATCAGCTGCCTGCAGTCTGAAGATTTTGCAACTTATTACTGCCAA
CAGTATAATAGTTATTCCCGGACGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K125-H1 CAR
(Sequence ID No. 143)
5′-GACATCCAGATGACCCAGTCTCCATCCTCCCTGTCTGCATCT
GTAGGAGACAGAGTCACCATCACTTGCCGGGCAAGTCAGAGCATT
AGCAGCTATTTAAATTGGTATCAGCAGAAACCAGGGAAAGCCCCT
AAGCTCCTGCTCTATGCTGCATCCAGATTGGAAAGTGGGGTCCCA
TCAAGGTTCAGCGGCAGTGGATCTGGGACAGAATTCACTCTCACC
ATCAGCAGCCTGCAGCCTGATGATTTTGCAACTTATTACTGCCAA
CAGTATAATAGTTATTCTCCGTGCACTTTCGGCCCTGGGACCAAA
GTGGATATCAAAGGCTCTACAAGCGGCTCTGGCAAGCCTGGATCT
GGCGAGGGAAGCACCAAGGGCCAGGTGCAGCTGCAGGAGTCGGGC
CCAGGACTGGTGAAGCCTTCGGATACCCTGTCCCTCACCTGTCTT
GTCTCTGGTGGCTCCATCAGTAGTAATTACTGGAGCTGGATCCGG
CAGGCCCCAGGGAAGGGACTGGAGTGGATTGGACATGTCTCCTAC
AGTGGGAGCACCAACTACAACCCCTCCCTCAAGAGTCGAGTTACC
ATATCAGTAGACACGTCTAAGAACCAGTTCTCCCTGAAGCTGAGC
TCTGTGACTGCCGCGGACACGGCCGTGTATTACTGTGCGAGAGAG
TCCTACTACTACTACGGTATGGACGTCTGGGGCCAAGGGACCACG
GTCACCGTCTCCTCAGCGGCCGCAATCGAAGTGATGTACCCCCCT
CCCTACCTGGACAACGAGAAGTCCAACGGCACCATTATCCACGTG
AAGGGAAAGCACCTGTGCCCCAGCCCTCTGTTCCCTGGCCCTAGC
AAGCCTTTCTGGGTGCTGGTGGTCGTGGGCGGAGTGCTGGCCTGT
TATAGCCTGCTCGTGACCGTGGCCTTCATCATCTTTTGGGTGCGC
AGCAAGCGGAGCCGGCTGCTGCACAGCGACTACATGAACATGACC
CCCAGACGGCCCGGACCCACCAGAAAGCACTACCAGCCTTACGCC
CCTCCCAGAGACTTCGCCGCCTACAGATCTCGAGTGAAGTTCAGC
AGAAGCGCCGACGCCCCTGCCTATCAGCAGGGCCAGAACCAGCTG
TACAACGAGCTGAACCTGGGCAGACGGGAAGAGTACGACGTGCTG
GACAAGCGGAGAGGCAGGGACCCTGAGATGGGCGGCAAGCCCAGA
AGAAAGAACCCCCAGGAAGGCCTGTATAACGAACTGCAGAAAGAC
AAGATGGCCGAGGCCTACAGCGAGATCGGCATGAAGGGCGAGCGG
AGAAGAGGCAAGGGCCACGATGGCCTGTACCAGGGCCTGAGCACC
GCCACCAAGGACACCTATGACGCCCTGCACATGCAGGCCCTGCCC
CCCAGA-3′
Polynucleotide Coding for K131-H1 CAR
(Sequence ID No. 144)
5′-GACATCCAGATGACCCAGTCTCCATCCTCCCTGTCTGCATCT
GTAGGGGACAGAGTCTCCATCACTTGCCGGGCAAGTCAGGACATT
AGCAGGTATTTAAATTGGTATCAGCAGAAACCAGGGAAAGCCCCT
AAGCTCCTGCTCTATGCTGCATCCAGATTGGAAAGTGGGGTCCCA
TCCAGGTTCAGTGGCAGTGGATCTGGGACAGACTTCACTCTCACC
ATCACCAGCCTGCAGCCTGATGACTTTGCAACTTATTACTGCCAA
CAGTATGATAATCTGATCACCTTCGGCCAAGGGACACGACTGGAG
ATTAAAGGCTCTACAAGCGGCTCTGGCAAGCCTGGATCTGGCGAG
GGAAGCACCAAGGGCCAGGTGCAGCTGCAGGAGTCGGGCCCAGGA
CTGGTGAAGCCTTCGGATACCCTGTCCCTCACCTGTCTTGTCTCT
GGTGGCTCCATCAGTAGTAATTACTGGAGCTGGATCCGGCAGGCC
CCAGGGAAGGGACTGGAGTGGATTGGACATGTCTCCTACAGTGGG
AGCACCAACTACAACCCCTCCCTCAAGAGTCGAGTTACCATATCA
GTAGACACGTCTAAGAACCAGTTCTCCCTGAAGCTGAGCTCTGTG
ACTGCCGCGGACACGGCCGTGTATTACTGTGCGAGAGAGTCCTAC
TACTACTACGGTATGGACGTCTGGGGCCAAGGGACCACGGTCACC
GTCTCCTCAGCGGCCGCAATCGAAGTGATGTACCCCCCTCCCTAC
CTGGACAACGAGAAGTCCAACGGCACCATTATCCACGTGAAGGGA
AAGCACCTGTGCCCCAGCCCTCTGTTCCCTGGCCCTAGCAAGCCT
TTCTGGGTGCTGGTGGTCGTGGGCGGAGTGCTGGCCTGTTATAGC
CTGCTCGTGACCGTGGCCTTCATCATCTTTTGGGTGCGCAGCAAG
CGGAGCCGGCTGCTGCACAGCGACTACATGAACATGACCCCCAGA
CGGCCCGGACCCACCAGAAAGCACTACCAGCCTTACGCCCCTCCC
AGAGACTTCGCCGCCTACAGATCTCGAGTGAAGTTCAGCAGAAGC
GCCGACGCCCCTGCCTATCAGCAGGGCCAGAACCAGCTGTACAAC
GAGCTGAACCTGGGCAGACGGGAAGAGTACGACGTGCTGGACAAG
CGGAGAGGCAGGGACCCTGAGATGGGCGGCAAGCCCAGAAGAAAG
AACCCCCAGGAAGGCCTGTATAACGAACTGCAGAAAGACAAGATG
GCCGAGGCCTACAGCGAGATCGGCATGAAGGGCGAGCGGAGAAGA
GGCAAGGGCCACGATGGCCTGTACCAGGGCCTGAGCACCGCCACC
AAGGACACCTATGACGCCCTGCACATGCAGGCCCTGCCCCCCAGA
-3′
Polynucleotide Coding for K145-H1 CAR
(Sequence ID No. 145)
5′-GACATCCAGATGACCCAGTCTCCATCCTCCCTGTCTGCATCT
GTAGGGGACAGAGTCTCCATCACTTGCCGGGCAAGTCAGGACATT
AGCAGGTATTTAAATTGGTATCAGCAGAAACCAGGGAAAGCCCCT
AAGCTCCTGCTCTATGCTGCATCCAGATTGGAAAGTGGGGTCCCA
TCCAGGTTCAGTGGCAGTGGATCTGGGACAGACTTCACTCTCACC
ATCACCAGCCTGCAGCCTGATGACTTTGCAACTTATTACTGCCAA
CAGTATAATAGTTATTCCCGGACGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K151-H1 CAR
(Sequence ID No. 146)
5′-GACATCCAGATGACCCAGTCTCCATCCTCCCTGTCTGCATCT
GTAGGGGACAGAGTCACCATCACTTGCCGGGCAAGTCAGAGCATT
AGCAGCTATTTAAATTGGTATCAGCAGAAACCAGGGAAAGCCCCT
AAGCTCCTGATCTATGCTGCATCCAGTTTGCAAAGTGGGGTCCCA
TCAAGGTTCAGTGGCAGTGGATCTGGGACAGACTTCACTCTCACC
ATCACCAGCCTGCAGCCTGATGACTTTGCAACTTATTACTGCCAA
CAGTATGATAATCTGATCACCTTCGGCCAAGGGACACGACTGGAG
ATTAAAGGCTCTACAAGCGGCTCTGGCAAGCCTGGATCTGGCGAG
GGAAGCACCAAGGGCCAGGTGCAGCTGCAGGAGTCGGGCCCAGGA
CTGGTGAAGCCTTCGGATACCCTGTCCCTCACCTGTCTTGTCTCT
GGTGGCTCCATCAGTAGTAATTACTGGAGCTGGATCCGGCAGGCC
CCAGGGAAGGGACTGGAGTGGATTGGACATGTCTCCTACAGTGGG
AGCACCAACTACAACCCCTCCCTCAAGAGTCGAGTTACCATATCA
GTAGACACGTCTAAGAACCAGTTCTCCCTGAAGCTGAGCTCTGTG
ACTGCCGCGGACACGGCCGTGTATTACTGTGCGAGAGAGTCCTAC
TACTACTACGGTATGGACGTCTGGGGCCAAGGGACCACGGTCACC
GTCTCCTCAGCGGCCGCAATCGAAGTGATGTACCCCCCTCCCTAC
CTGGACAACGAGAAGTCCAACGGCACCATTATCCACGTGAAGGGA
AAGCACCTGTGCCCCAGCCCTCTGTTCCCTGGCCCTAGCAAGCCT
TTCTGGGTGCTGGTGGTCGTGGGCGGAGTGCTGGCCTGTTATAGC
CTGCTCGTGACCGTGGCCTTCATCATCTTTTGGGTGCGCAGCAAG
CGGAGCCGGCTGCTGCACAGCGACTACATGAACATGACCCCCAGA
CGGCCCGGACCCACCAGAAAGCACTACCAGCCTTACGCCCCTCCC
AGAGACTTCGCCGCCTACAGATCTCGAGTGAAGTTCAGCAGAAGC
GCCGACGCCCCTGCCTATCAGCAGGGCCAGAACCAGCTGTACAAC
GAGCTGAACCTGGGCAGACGGGAAGAGTACGACGTGCTGGACAAG
CGGAGAGGCAGGGACCCTGAGATGGGCGGCAAGCCCAGAAGAAAG
AACCCCCAGGAAGGCCTGTATAACGAACTGCAGAAAGACAAGATG
GCCGAGGCCTACAGCGAGATCGGCATGAAGGGCGAGCGGAGAAGA
GGCAAGGGCCACGATGGCCTGTACCAGGGCCTGAGCACCGCCACC
AAGGACACCTATGACGCCCTGCACATGCAGGCCCTGCCCCCCAGA
-3′
Polynucleotide Coding for K160-H1 CAR
(Sequence ID No. 147)
5′-GAAATTGTGTTGACGCAGTCTCCAGCCACCCTGTCTGTGTCT
CCAGGGGAAAGAGCCACCCTCTCCTGCAGGGCCAGTCAGAGTGTT
AGCAGCAACTTAGCCTGGTACCAGCAGAAACCTGGCCAGGCTCCC
AGGCTCCTCATCTATGGTGCATCCACCAGGGCCACTGGTATCCCA
GCCAGGTTCAGTGGCAGTGGGTCTGGGACAGAGTTCACTCTCACC
ATCAGCAGACTGGAGCCTGAAGATTTTGCAACCTATTACTGCCAA
CAGTATAATAGTTATTCCCGGACGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for K173-H1 CAR
(Sequence ID No. 148)
5′-GACATCCAGATGACCCAGTCTCCATCCTCCCTGTCTGCATCT
GTAGGAGACAGAGTCACCATCACTTGCCGGGCAAGTCAGAGCATT
AGCAGCTATTTAAATTGGTATCAGCAGAAAGCAGGGAAAGCCCCT
AAGCTTCTGATCTATGCTGCATCCAGTTTGCAAAGTGGGGTCCCA
TCAAGGTTCAGCGGCAGTGGATCTGGGACAGATTTCACTCTCACC
ATCAGCTGCCTGCAGTCTGAAGATGTTGCAACCTATTACTGCCAA
CAGTATGAAAGTTATTCCCGGACGTTCGGCCAAGGGACCAAGGTG
GAAATCAAAGGCTCTACAAGCGGCTCTGGCAAGCCTGGATCTGGC
GAGGGAAGCACCAAGGGCCAGGTGCAGCTGCAGGAGTCGGGCCCA
GGACTGGTGAAGCCTTCGGATACCCTGTCCCTCACCTGTCTTGTC
TCTGGTGGCTCCATCAGTAGTAATTACTGGAGCTGGATCCGGCAG
GCCCCAGGGAAGGGACTGGAGTGGATTGGACATGTCTCCTACAGT
GGGAGCACCAACTACAACCCCTCCCTCAAGAGTCGAGTTACCATA
TCAGTAGACACGTCTAAGAACCAGTTCTCCCTGAAGCTGAGCTCT
GTGACTGCCGCGGACACGGCCGTGTATTACTGTGCGAGAGAGTCC
TACTACTACTACGGTATGGACGTCTGGGGCCAAGGGACCACGGTC
ACCGTCTCCTCAGCGGCCGCAATCGAAGTGATGTACCCCCCTCCC
TACCTGGACAACGAGAAGTCCAACGGCACCATTATCCACGTGAAG
GGAAAGCACCTGTGCCCCAGCCCTCTGTTCCCTGGCCCTAGCAAG
CCTTTCTGGGTGCTGGTGGTCGTGGGCGGAGTGCTGGCCTGTTAT
AGCCTGCTCGTGACCGTGGCCTTCATCATCTTTTGGGTGCGCAGC
AAGCGGAGCCGGCTGCTGCACAGCGACTACATGAACATGACCCCC
AGACGGCCCGGACCCACCAGAAAGCACTACCAGCCTTACGCCCCT
CCCAGAGACTTCGCCGCCTACAGATCTCGAGTGAAGTTCAGCAGA
AGCGCCGACGCCCCTGCCTATCAGCAGGGCCAGAACCAGCTGTAC
AACGAGCTGAACCTGGGCAGACGGGAAGAGTACGACGTGCTGGAC
AAGCGGAGAGGCAGGGACCCTGAGATGGGCGGCAAGCCCAGAAGA
AAGAACCCCCAGGAAGGCCTGTATAACGAACTGCAGAAAGACAAG
ATGGCCGAGGCCTACAGCGAGATCGGCATGAAGGGCGAGCGGAGA
AGAGGCAAGGGCCACGATGGCCTGTACCAGGGCCTGAGCACCGCC
ACCAAGGACACCTATGACGCCCTGCACATGCAGGCCCTGCCCCCC
AGA-3′
Polynucleotide Coding for L1-3M4E5H CAR
(Sequence ID No. 149)
5′-CAGTCTGCCCTGACTCAGCCTCCCTCCGCGTCCGGGTCTCCT
GGACAGTCAGTCACCATCTCCTGCACTGGAACCAGCAGTGACGTT
GGTGGTTATGACTTTGTCTCCTGGTACCAACAGCACCCAGGCGAA
GCCCCCAAACTCCTCGTTTATGATGTCAATAACCGGCCCTCAGGG
GTTTCTAATCGCTTCTCTGGCTCCAAGTCTGGCAACACGGCCTCC
CTGACCATCTCTGGGCTCCAGGCTGAGGACGAGGGTGACTATTAC
TGCAGCTCATATGCAGGCAGCAACAGCGTCTTCGGAACTGGGACC
AAGGTCACCGTCCTAGGCTCTACAAGCGGCTCTGGCAAGCCTGGA
TCTGGCGAGGGAAGCACCAAGGGCGAAGTGCAGCTGCTGGAATCT
GGCGGCGGACTGGTGCAGCCTGGCGGATCTCTGAGACTGAGCTGT
GCCGCCAGCGGCTTCACCTTCAGCACCTACCAGATGAGCTGGGTG
CGCCAGGCCCCTGGCAAAGGACTGGAATGGGTGTCCGGCATCGTG
TCCAGCGGCGGCTCTACAGCCTACGCCGATAGCGTGAAGGGCCGG
TTCACCATCAGCCGGGACAACAGCAAGAACACCCTGTACCTGCAG
ATGAACAGCCTGAGAGCCGAGGACACCGCCGTGTACTATTGTGCC
GGGGAGCTGCTGCCCTACTACGGCATGGATGTGTGGGGCCAGGGC
ACCACCGTGACAGTGTCCTCAGCGGCCGCAATCGAAGTGATGTAC
CCCCCTCCCTACCTGGACAACGAGAAGTCCAACGGCACCATTATC
CACGTGAAGGGAAAGCACCTGTGCCCCAGCCCTCTGTTCCCTGGC
CCTAGCAAGCCTTTCTGGGTGCTGGTGGTCGTGGGCGGAGTGCTG
GCCTGTTATAGCCTGCTCGTGACCGTGGCCTTCATCATCTTTTGG
GTGCGCAGCAAGCGGAGCCGGCTGCTGCACAGCGACTACATGAAC
ATGACCCCCAGACGGCCCGGACCCACCAGAAAGCACTACCAGCCT
TACGCCCCTCCCAGAGACTTCGCCGCCTACAGATCTCGAGTGAAG
TTCAGCAGAAGCGCCGACGCCCCTGCCTATCAGCAGGGCCAGAAC
CAGCTGTACAACGAGCTGAACCTGGGCAGACGGGAAGAGTACGAC
GTGCTGGACAAGCGGAGAGGCAGGGACCCTGAGATGGGCGGCAAG
CCCAGAAGAAAGAACCCCCAGGAAGGCCTGTATAACGAACTGCAG
AAAGACAAGATGGCCGAGGCCTACAGCGAGATCGGCATGAAGGGC
GAGCGGAGAAGAGGCAAGGGCCACGATGGCCTGTACCAGGGCCTG
AGCACCGCCACCAAGGACACCTATGACGCCCTGCACATGCAGGCC
CTGCCCCCCAGA-3′
Polynucleotide Coding for 3M4E5H-L1 CAR
(Sequence ID No. 150)
5′-GAAGTGCAGCTGCTGGAATCTGGCGGCGGACTGGTGCAGCCT
GGCGGATCTCTGAGACTGAGCTGTGCCGCCAGCGGCTTCACCTTC
AGCACCTACCAGATGAGCTGGGTGCGCCAGGCCCCTGGCAAAGGA
CTGGAATGGGTGTCCGGCATCGTGTCCAGCGGCGGCTCTACAGCC
TACGCCGATAGCGTGAAGGGCCGGTTCACCATCAGCCGGGACAAC
AGCAAGAACACCCTGTACCTGCAGATGAACAGCCTGAGAGCCGAG
GACACCGCCGTGTACTATTGTGCCGGGGAGCTGCTGCCCTACTAC
GGCATGGATGTGTGGGGCCAGGGCACCACCGTGACAGTGTCCTCA
GGCTCTACAAGCGGCTCTGGCAAGCCTGGATCTGGCGAGGGAAGC
ACCAAGGGCCAGTCTGCCCTGACTCAGCCTCCCTCCGCGTCCGGG
TCTCCTGGACAGTCAGTCACCATCTCCTGCACTGGAACCAGCAGT
GACGTTGGTGGTTATGACTTTGTCTCCTGGTACCAACAGCACCCA
GGCGAAGCCCCCAAACTCCTCGTTTATGATGTCAATAACCGGCCC
TCAGGGGTTTCTAATCGCTTCTCTGGCTCCAAGTCTGGCAACACG
GCCTCCCTGACCATCTCTGGGCTCCAGGCTGAGGACGAGGGTGAC
TATTACTGCAGCTCATATGCAGGCAGCAACAGCGTCTTCGGAACT
GGGACCAAGGTCACCGTCCTAGCGGCCGCAATCGAAGTGATGTAC
CCCCCTCCCTACCTGGACAACGAGAAGTCCAACGGCACCATTATC
CACGTGAAGGGAAAGCACCTGTGCCCCAGCCCTCTGTTCCCTGGC
CCTAGCAAGCCTTTCTGGGTGCTGGTGGTCGTGGGCGGAGTGCTG
GCCTGTTATAGCCTGCTCGTGACCGTGGCCTTCATCATCTTTTGG
GTGCGCAGCAAGCGGAGCCGGCTGCTGCACAGCGACTACATGAAC
ATGACCCCCAGACGGCCCGGACCCACCAGAAAGCACTACCAGCCT
TACGCCCCTCCCAGAGACTTCGCCGCCTACAGATCTCGAGTGAAG
TTCAGCAGAAGCGCCGACGCCCCTGCCTATCAGCAGGGCCAGAAC
CAGCTGTACAACGAGCTGAACCTGGGCAGACGGGAAGAGTACGAC
GTGCTGGACAAGCGGAGAGGCAGGGACCCTGAGATGGGCGGCAAG
CCCAGAAGAAAGAACCCCCAGGAAGGCCTGTATAACGAACTGCAG
AAAGACAAGATGGCCGAGGCCTACAGCGAGATCGGCATGAAGGGC
GAGCGGAGAAGAGGCAAGGGCCACGATGGCCTGTACCAGGGCCTG
AGCACCGCCACCAAGGACACCTATGACGCCCTGCACATGCAGGCC
CTGCCCCCCAGA-3′
Polynucleotide Coding for L66-3M4E5H CAR
(Sequence ID No. 151)
5′-CAGTCTGCCCTGACTCAGCCTGCCTCCGTGTCTGGGTCTCCT
GGACAGTCGATCACCATCTCCTGCACTGGGACCAGCAGTGACGTT
GGTGGTTATGAATTTGTCTCCTGGTACCAACAACACCCAGGCAGC
GCCCCCAAACTCATTATTTATGACGTAATAGAGCGTCCCTTCGGT
GTCTCCTATCGGTTCTCTGCCTCCAAGTCAGGCAACACGGCCTCC
CTGACGATCTCTGGGCTCCAGGGTGAAGACGAGGCTGATTACTTC
TGCAGCTCATATACAAGCAGCAGCACTTATGTCTTCGGAACTGGG
ACCAAGGTCACCGTCCTAGGCTCTACAAGCGGCTCTGGCAAGCCT
GGATCTGGCGAGGGAAGCACCAAGGGCGAAGTGCAGCTGCTGGAA
TCTGGCGGCGGACTGGTGCAGCCTGGCGGATCTCTGAGACTGAGC
TGTGCCGCCAGCGGCTTCACCTTCAGCACCTACCAGATGAGCTGG
GTGCGCCAGGCCCCTGGCAAAGGACTGGAATGGGTGTCCGGCATC
GTGTCCAGCGGCGGCTCTACAGCCTACGCCGATAGCGTGAAGGGC
CGGTTCACCATCAGCCGGGACAACAGCAAGAACACCCTGTACCTG
CAGATGAACAGCCTGAGAGCCGAGGACACCGCCGTGTACTATTGT
GCCGGGGAGCTGCTGCCCTACTACGGCATGGATGTGTGGGGCCAG
GGCACCACCGTGACAGTGTCCTCAGCGGCCGCAATCGAAGTGATG
TACCCCCCTCCCTACCTGGACAACGAGAAGTCCAACGGCACCATT
ATCCACGTGAAGGGAAAGCACCTGTGCCCCAGCCCTCTGTTCCCT
GGCCCTAGCAAGCCTTTCTGGGTGCTGGTGGTCGTGGGCGGAGTG
CTGGCCTGTTATAGCCTGCTCGTGACCGTGGCCTTCATCATCTTT
TGGGTGCGCAGCAAGCGGAGCCGGCTGCTGCACAGCGACTACATG
AACATGACCCCCAGACGGCCCGGACCCACCAGAAAGCACTACCAG
CCTTACGCCCCTCCCAGAGACTTCGCCGCCTACAGATCTCGAGTG
AAGTTCAGCAGAAGCGCCGACGCCCCTGCCTATCAGCAGGGCCAG
AACCAGCTGTACAACGAGCTGAACCTGGGCAGACGGGAAGAGTAC
GACGTGCTGGACAAGCGGAGAGGCAGGGACCCTGAGATGGGCGGC
AAGCCCAGAAGAAAGAACCCCCAGGAAGGCCTGTATAACGAACTG
CAGAAAGACAAGATGGCCGAGGCCTACAGCGAGATCGGCATGAAG
GGCGAGCGGAGAAGAGGCAAGGGCCACGATGGCCTGTACCAGGGC
CTGAGCACCGCCACCAAGGACACCTATGACGCCCTGCACATGCAG
GCCCTGCCCCCCAGA-3′
Polynucleotide Coding for L73-3M4E5H CAR
(Sequence ID No. 152)
5′-CAGTCTGCCCTGACTCAGCCTGCCTCCGTGTCTGGGTCTCCT
GGACAGTCGATCACCATCTCCTGCACTGGAACCGGCAGTGACGTT
GGTGCTTATGACTATGTCTCCTGGTACCAACATCACCCAGGCAGA
GCCCCCAGACTCATCATTCGTGATGTCAGTGTGCGGCCCTCAGGG
GTCCCTGATCGCTTCTCTGGCTCCAAGTCTGGCAACACGGCCTCC
CTGACCATCTCTGGGCTCCAGGCTGAGGACGAGGCTGATTATTAC
TGCTCCTCATATTCAGGCAGCAGCACTTGGGTGTTCGGCGGGGGG
ACCAAGCTGACCGTCCTAGGCTCTACAAGCGGCTCTGGCAAGCCT
GGATCTGGCGAGGGAAGCACCAAGGGCGAAGTGCAGCTGCTGGAA
TCTGGCGGCGGACTGGTGCAGCCTGGCGGATCTCTGAGACTGAGC
TGTGCCGCCAGCGGCTTCACCTTCAGCACCTACCAGATGAGCTGG
GTGCGCCAGGCCCCTGGCAAAGGACTGGAATGGGTGTCCGGCATC
GTGTCCAGCGGCGGCTCTACAGCCTACGCCGATAGCGTGAAGGGC
CGGTTCACCATCAGCCGGGACAACAGCAAGAACACCCTGTACCTG
CAGATGAACAGCCTGAGAGCCGAGGACACCGCCGTGTACTATTGT
GCCGGGGAGCTGCTGCCCTACTACGGCATGGATGTGTGGGGCCAG
GGCACCACCGTGACAGTGTCCTCAGCGGCCGCAATCGAAGTGATG
TACCCCCCTCCCTACCTGGACAACGAGAAGTCCAACGGCACCATT
ATCCACGTGAAGGGAAAGCACCTGTGCCCCAGCCCTCTGTTCCCT
GGCCCTAGCAAGCCTTTCTGGGTGCTGGTGGTCGTGGGCGGAGTG
CTGGCCTGTTATAGCCTGCTCGTGACCGTGGCCTTCATCATCTTT
TGGGTGCGCAGCAAGCGGAGCCGGCTGCTGCACAGCGACTACATG
AACATGACCCCCAGACGGCCCGGACCCACCAGAAAGCACTACCAG
CCTTACGCCCCTCCCAGAGACTTCGCCGCCTACAGATCTCGAGTG
AAGTTCAGCAGAAGCGCCGACGCCCCTGCCTATCAGCAGGGCCAG
AACCAGCTGTACAACGAGCTGAACCTGGGCAGACGGGAAGAGTAC
GACGTGCTGGACAAGCGGAGAGGCAGGGACCCTGAGATGGGCGGC
AAGCCCAGAAGAAAGAACCCCCAGGAAGGCCTGTATAACGAACTG
CAGAAAGACAAGATGGCCGAGGCCTACAGCGAGATCGGCATGAAG
GGCGAGCGGAGAAGAGGCAAGGGCCACGATGGCCTGTACCAGGGC
CTGAGCACCGCCACCAAGGACACCTATGACGCCCTGCACATGCAG
GCCCTGCCCCCCAGA-3′
Polynucleotide Coding for L80-3M4E5H CAR
(Sequence ID No. 153)
5′-CAGTCTGCCCTGACTCAGCCTGCCTCCGTGTCTGGGTCTCCT
GGACAGTCGATCACCATCTCCTGCACTGGAACCAGCAGTGATGTT
GGGAGTTATAACCTTGTCTCCTGGTACCAACAACACCCAGGCAAA
GCCCCCAAACTCATGATTTATGATGTCAGTAATCGGCCCTCAGGG
GTTTCTTATCGCTTCTCTGGCTCCAAGTCTGGCAACACGGCCTCC
CTGACCATCTCTGGGCTCCAGGCTGAGGACGAGGCTGATTATTAC
TGCAGCTCATATACAAGCAGCAGCACTTTCGCGGTGTTCGGCGGA
GGGACCCAGCTGACCGTCCTCGGCTCTACAAGCGGCTCTGGCAAG
CCTGGATCTGGCGAGGGAAGCACCAAGGGCGAAGTGCAGCTGCTG
GAATCTGGCGGCGGACTGGTGCAGCCTGGCGGATCTCTGAGACTG
AGCTGTGCCGCCAGCGGCTTCACCTTCAGCACCTACCAGATGAGC
TGGGTGCGCCAGGCCCCTGGCAAAGGACTGGAATGGGTGTCCGGC
ATCGTGTCCAGCGGCGGCTCTACAGCCTACGCCGATAGCGTGAAG
GGCCGGTTCACCATCAGCCGGGACAACAGCAAGAACACCCTGTAC
CTGCAGATGAACAGCCTGAGAGCCGAGGACACCGCCGTGTACTAT
TGTGCCGGGGAGCTGCTGCCCTACTACGGCATGGATGTGTGGGGC
CAGGGCACCACCGTGACAGTGTCCTCAGCGGCCGCAATCGAAGTG
ATGTACCCCCCTCCCTACCTGGACAACGAGAAGTCCAACGGCACC
ATTATCCACGTGAAGGGAAAGCACCTGTGCCCCAGCCCTCTGTTC
CCTGGCCCTAGCAAGCCTTTCTGGGTGCTGGTGGTCGTGGGCGGA
GTGCTGGCCTGTTATAGCCTGCTCGTGACCGTGGCCTTCATCATC
TTTTGGGTGCGCAGCAAGCGGAGCCGGCTGCTGCACAGCGACTAC
ATGAACATGACCCCCAGACGGCCCGGACCCACCAGAAAGCACTAC
CAGCCTTACGCCCCTCCCAGAGACTTCGCCGCCTACAGATCTCGA
GTGAAGTTCAGCAGAAGCGCCGACGCCCCTGCCTATCAGCAGGGC
CAGAACCAGCTGTACAACGAGCTGAACCTGGGCAGACGGGAAGAG
TACGACGTGCTGGACAAGCGGAGAGGCAGGGACCCTGAGATGGGC
GGCAAGCCCAGAAGAAAGAACCCCCAGGAAGGCCTGTATAACGAA
CTGCAGAAAGACAAGATGGCCGAGGCCTACAGCGAGATCGGCATG
AAGGGCGAGCGGAGAAGAGGCAAGGGCCACGATGGCCTGTACCAG
GGCCTGAGCACCGCCACCAAGGACACCTATGACGCCCTGCACATG
CAGGCCCTGCCCCCCAGA-3′
Polynucleotide Coding for L88-3M4E5H CAR
(Sequence ID No. 154)
5′-CAATCTGCCCTGCCTCAGCCTGCCTCCGTGTCTGGGTCTCCT
GGACAGTCAGTCACCATCTCCTGCACTGGAACCAGCAGTGATGTT
GGTGGTTATAACTATGTCTCCTGGTACCAACAGCACCCAGGCAAA
GCCCCCAAACTCATGATTTATGATGTCAGTAAGCGGCCCTCAGGG
GTCCCTGATCGCTTCTCTGGTTCCAAGTCTGGCAACACGGCCTCC
CTGACCGTCTCTGGGCTCCAGGCTGAGGATGAGGCTGATTATTTC
TGCTGTTCGTATGCAGGCGGCTATTATGTCTTCGGAACTGGGACC
AAGCTGACCGTCCTAGGCTCTACAAGCGGCTCTGGCAAGCCTGGA
TCTGGCGAGGGAAGCACCAAGGGCGAAGTGCAGCTGCTGGAATCT
GGCGGCGGACTGGTGCAGCCTGGCGGATCTCTGAGACTGAGCTGT
GCCGCCAGCGGCTTCACCTTCAGCACCTACCAGATGAGCTGGGTG
CGCCAGGCCCCTGGCAAAGGACTGGAATGGGTGTCCGGCATCGTG
TCCAGCGGCGGCTCTACAGCCTACGCCGATAGCGTGAAGGGCCGG
TTCACCATCAGCCGGGACAACAGCAAGAACACCCTGTACCTGCAG
ATGAACAGCCTGAGAGCCGAGGACACCGCCGTGTACTATTGTGCC
GGGGAGCTGCTGCCCTACTACGGCATGGATGTGTGGGGCCAGGGC
ACCACCGTGACAGTGTCCTCAGCGGCCGCAATCGAAGTGATGTAC
CCCCCTCCCTACCTGGACAACGAGAAGTCCAACGGCACCATTATC
CACGTGAAGGGAAAGCACCTGTGCCCCAGCCCTCTGTTCCCTGGC
CCTAGCAAGCCTTTCTGGGTGCTGGTGGTCGTGGGCGGAGTGCTG
GCCTGTTATAGCCTGCTCGTGACCGTGGCCTTCATCATCTTTTGG
GTGCGCAGCAAGCGGAGCCGGCTGCTGCACAGCGACTACATGAAC
ATGACCCCCAGACGGCCCGGACCCACCAGAAAGCACTACCAGCCT
TACGCCCCTCCCAGAGACTTCGCCGCCTACAGATCTCGAGTGAAG
TTCAGCAGAAGCGCCGACGCCCCTGCCTATCAGCAGGGCCAGAAC
CAGCTGTACAACGAGCTGAACCTGGGCAGACGGGAAGAGTACGAC
GTGCTGGACAAGCGGAGAGGCAGGGACCCTGAGATGGGCGGCAAG
CCCAGAAGAAAGAACCCCCAGGAAGGCCTGTATAACGAACTGCAG
AAAGACAAGATGGCCGAGGCCTACAGCGAGATCGGCATGAAGGGC
GAGCGGAGAAGAGGCAAGGGCCACGATGGCCTGTACCAGGGCCTG
AGCACCGCCACCAAGGACACCTATGACGCCCTGCACATGCAGGCC
CTGCCCCCCAGA-3′
Polynucleotide Coding for L102-3M4E5H CAR
(Sequence ID No. 155)
5′-CAGTCTGCCCTGACTCAGCCTCCCTCCGCGTCCGGGTCTCCT
GGACAGTCAGTCACCATCTCCTGCACTGGAACCAGCAGTGACGTT
GGTGGTTATAACTATGTCTCCTGGTACCAACAGCACCCAGGCAAA
GCCCCCAAACTCATGATTTATGATGTCAGTAAGCGGCCCTCAGGG
GTCCCTGATCGCTTCTCTGGCTCCAAGTCTGGCAACACGGCCTCC
CTGACCATCTCTGGGCTCCAGACTGAGGACGAGGCTGATTACTAT
TGCAGCTCATATGCAGGCAGCGGCAGCACCCCCTTTGTCTTCGGA
ACTGGGACCAAGCTGACCGTCCTAGGCTCTACAAGCGGCTCTGGC
AAGCCTGGATCTGGCGAGGGAAGCACCAAGGGCGAAGTGCAGCTG
CTGGAATCTGGCGGCGGACTGGTGCAGCCTGGCGGATCTCTGAGA
CTGAGCTGTGCCGCCAGCGGCTTCACCTTCAGCACCTACCAGATG
AGCTGGGTGCGCCAGGCCCCTGGCAAAGGACTGGAATGGGTGTCC
GGCATCGTGTCCAGCGGCGGCTCTACAGCCTACGCCGATAGCGTG
AAGGGCCGGTTCACCATCAGCCGGGACAACAGCAAGAACACCCTG
TACCTGCAGATGAACAGCCTGAGAGCCGAGGACACCGCCGTGTAC
TATTGTGCCGGGGAGCTGCTGCCCTACTACGGCATGGATGTGTGG
GGCCAGGGCACCACCGTGACAGTGTCCTCAGCGGCCGCAATCGAA
GTGATGTACCCCCCTCCCTACCTGGACAACGAGAAGTCCAACGGC
ACCATTATCCACGTGAAGGGAAAGCACCTGTGCCCCAGCCCTCTG
TTCCCTGGCCCTAGCAAGCCTTTCTGGGTGCTGGTGGTCGTGGGC
GGAGTGCTGGCCTGTTATAGCCTGCTCGTGACCGTGGCCTTCATC
ATCTTTTGGGTGCGCAGCAAGCGGAGCCGGCTGCTGCACAGCGAC
TACATGAACATGACCCCCAGACGGCCCGGACCCACCAGAAAGCAC
TACCAGCCTTACGCCCCTCCCAGAGACTTCGCCGCCTACAGATCT
CGAGTGAAGTTCAGCAGAAGCGCCGACGCCCCTGCCTATCAGCAG
GGCCAGAACCAGCTGTACAACGAGCTGAACCTGGGCAGACGGGAA
GAGTACGACGTGCTGGACAAGCGGAGAGGCAGGGACCCTGAGATG
GGCGGCAAGCCCAGAAGAAAGAACCCCCAGGAAGGCCTGTATAAC
GAACTGCAGAAAGACAAGATGGCCGAGGCCTACAGCGAGATCGGC
ATGAAGGGCGAGCGGAGAAGAGGCAAGGGCCACGATGGCCTGTAC
CAGGGCCTGAGCACCGCCACCAAGGACACCTATGACGCCCTGCAC
ATGCAGGCCCTGCCCCCCAGA-3′
Polynucleotide Coding for L124-3M4E5H CAR
(Sequence ID No. 156)
5′-CAGTCTGCCCTGACTCAGCCTGCCTCCGTGTCTGGGTCTCCT
GGACAGTCGATCACCATCTCCTGCACTGGAACCAGCAGTGACGTT
GGTGGTTATAACTATGTCTCCTGGTACCAACAACACCCAGGCAAA
GCCCCCAAACTCATGATTTATGATGTCAGTAATCGGCCCTCAAGG
GTCCCTGATCGCTTCGCTGGCTCCAAGTCTGGCAACACGGCCTCC
CTGACCATCTCTGGGCTCCAGGCTGAAGACGAGGCTGATTATTAC
TGCTGCTCATATGCCGGCAGACGTTATGTGTTCGGAACTGGGACC
AAGCTGACCGTCCTAGGCTCTACAAGCGGCTCTGGCAAGCCTGGA
TCTGGCGAGGGAAGCACCAAGGGCGAAGTGCAGCTGCTGGAATCT
GGCGGCGGACTGGTGCAGCCTGGCGGATCTCTGAGACTGAGCTGT
GCCGCCAGCGGCTTCACCTTCAGCACCTACCAGATGAGCTGGGTG
CGCCAGGCCCCTGGCAAAGGACTGGAATGGGTGTCCGGCATCGTG
TCCAGCGGCGGCTCTACAGCCTACGCCGATAGCGTGAAGGGCCGG
TTCACCATCAGCCGGGACAACAGCAAGAACACCCTGTACCTGCAG
ATGAACAGCCTGAGAGCCGAGGACACCGCCGTGTACTATTGTGCC
GGGGAGCTGCTGCCCTACTACGGCATGGATGTGTGGGGCCAGGGC
ACCACCGTGACAGTGTCCTCAGCGGCCGCAATCGAAGTGATGTAC
CCCCCTCCCTACCTGGACAACGAGAAGTCCAACGGCACCATTATC
CACGTGAAGGGAAAGCACCTGTGCCCCAGCCCTCTGTTCCCTGGC
CCTAGCAAGCCTTTCTGGGTGCTGGTGGTCGTGGGCGGAGTGCTG
GCCTGTTATAGCCTGCTCGTGACCGTGGCCTTCATCATCTTTTGG
GTGCGCAGCAAGCGGAGCCGGCTGCTGCACAGCGACTACATGAAC
ATGACCCCCAGACGGCCCGGACCCACCAGAAAGCACTACCAGCCT
TACGCCCCTCCCAGAGACTTCGCCGCCTACAGATCTCGAGTGAAG
TTCAGCAGAAGCGCCGACGCCCCTGCCTATCAGCAGGGCCAGAAC
CAGCTGTACAACGAGCTGAACCTGGGCAGACGGGAAGAGTACGAC
GTGCTGGACAAGCGGAGAGGCAGGGACCCTGAGATGGGCGGCAAG
CCCAGAAGAAAGAACCCCCAGGAAGGCCTGTATAACGAACTGCAG
AAAGACAAGATGGCCGAGGCCTACAGCGAGATCGGCATGAAGGGC
GAGCGGAGAAGAGGCAAGGGCCACGATGGCCTGTACCAGGGCCTG
AGCACCGCCACCAAGGACACCTATGACGCCCTGCACATGCAGGCC
CTGCCCCCCAGA-3′
Polynucleotide Coding for H73-3M4E5L CAR
(Sequence ID No. 157)
5′-CAGCTGCAGCTGCAGGAGTCGGGCCCAGGACTGGTGAAGCCT
TCACAGACCCTGTCCCTCACCTGCACTGTCTCTGGTGGCTCCATC
AGTAGTTACTACTGGAGCTGGATCCGGCAGCCCCCAGGGAAGGGA
CTGGAGTGGATTGGGGAAATCAATCATAGTGGAAGCACCAACTAC
AACCCGTCCCTCAAGAGTCGAGTCACCATATCAGTAGACACGTCT
AAGAACCAGTTCTCCCTGAAGCTGAGCTCTGTGACTGCCGCGGAC
ACGGCCGTGTATTACTGTGCGAGGTGCCCTATCTACTACTACGGT
ATGGACGTCTGGGGCCAAGGGACCACGGTCACCGTCTCCTCAGGC
TCTACAAGCGGCTCTGGCAAGCCTGGATCTGGCGAGGGAAGCACC
AAGGGCCAGAGCGAGCTGACACAGCCTAGATCCGTGTCTGGCAGC
CCTGGCCAGAGCGTGACCATCAGCTGTACCGGCACCAGCAGAGAT
GTGGGCGGCTACAACTACGTGTCCTGGTATCAGCAGCATCCCGGC
AAGGCCCCCAAGCTGATCATCCACGACGTGATCGAGCGGAGCAGC
GGCGTGCCCGATAGATTCAGCGGCAGCAAGAGCGGCAACACCGCC
AGCCTGACAATCAGCGGACTGCAGGCCGAGGACGAGGCCGACTAC
TACTGTTGGAGCTTCGCCGGCAGCTACTACGTGTTCGGCACCGGC
ACCGATGTGACCGTGCTGGCGGCCGCAATCGAAGTGATGTACCCC
CCTCCCTACCTGGACAACGAGAAGTCCAACGGCACCATTATCCAC
GTGAAGGGAAAGCACCTGTGCCCCAGCCCTCTGTTCCCTGGCCCT
AGCAAGCCTTTCTGGGTGCTGGTGGTCGTGGGCGGAGTGCTGGCC
TGTTATAGCCTGCTCGTGACCGTGGCCTTCATCATCTTTTGGGTG
CGCAGCAAGCGGAGCCGGCTGCTGCACAGCGACTACATGAACATG
ACCCCCAGACGGCCCGGACCCACCAGAAAGCACTACCAGCCTTAC
GCCCCTCCCAGAGACTTCGCCGCCTACAGATCTCGAGTGAAGTTC
AGCAGAAGCGCCGACGCCCCTGCCTATCAGCAGGGCCAGAACCAG
CTGTACAACGAGCTGAACCTGGGCAGACGGGAAGAGTACGACGTG
CTGGACAAGCGGAGAGGCAGGGACCCTGAGATGGGCGGCAAGCCC
AGAAGAAAGAACCCCCAGGAAGGCCTGTATAACGAACTGCAGAAA
GACAAGATGGCCGAGGCCTACAGCGAGATCGGCATGAAGGGCGAG
CGGAGAAGAGGCAAGGGCCACGATGGCCTGTACCAGGGCCTGAGC
ACCGCCACCAAGGACACCTATGACGCCCTGCACATGCAGGCCCTG
CCCCCCAGA-3′

Another specific example of the nucleic acid of the present invention is a polynucleotide of (ca) below.

(ca) A polynucleotide (nucleic acid) of (ca1), (ca2), (ca3), (ca4), (ca5), or (ca6) below:
(ca1) a polynucleotide coding for a polypeptide consisting of the amino acid sequence of any one of Sequence ID Nos. 272 to 284,
(ca2) a polynucleotide coding for a polypeptide consisting of an amino acid sequence having 80% or more identity to the amino acid sequence of any one of Sequence ID Nos. 272 to 284, the polypeptide being capable of binding to CD19,
(ca3) a polynucleotide coding for a polypeptide consisting of an amino acid sequence consisting of the amino acid sequence of any one of Sequence ID Nos. 272 to 284 with deletion, substitution, insertion, and/or addition of one or several amino acids, the polypeptide being capable of binding to CD19,
(ca4) a polynucleotide consisting of the base sequence of any one of Sequence ID Nos. 298 to 310,
(ca5) a polynucleotide consisting of a base sequence having 80% or more identity to the base sequence of any one of Sequence ID Nos. 298 to 310, and coding for a polypeptide capable of binding to CD19, and
(ca6) a polynucleotide consisting of a base sequence consisting of the base sequence of any one of Sequence ID Nos. 298 to 310 with deletion, substitution, insertion, and/or addition of one or several bases, and coding for a polypeptide capable of binding to CD19.

The polynucleotides of (ca1) to (ca3) above are nucleic acids coding for the polypeptides of (CA1) to (CA3) above, respectively, and the descriptions of the polypeptides of (CA1) to (CA3) above can be applied.

In the polynucleotide of (ca4) above, each of the base sequences of Sequence ID Nos. 298 to 310 is a polynucleotide coding for a polypeptide that includes an amino acid sequence that includes a combination of the heavy-chain variable region and the light-chain variable region that corresponds to any one of Combinations (LA) to (LM) for the second antibody of the present invention or the like, for example. In the polynucleotides of (ca5) and (ca6) above, the polynucleotides coding for the amino acid sequences that correspond to the CDRH1, the CDRH2, the CDRH3, the CDRL1, the CDRL2, and the CDRL3 are conserved in the base sequences of Sequence ID Nos. 298 to 310, for example. Regarding the base sequences coding for the amino acid sequences of the CDRs in the base sequences of Sequence ID Nos. 298 to 310, the amino acid sequences of the CDRs included in the amino acid sequences that include the combinations of the heavy-chain variable region and the light-chain variable region that correspond to Combinations (LA) to (LM) for the second antibody of the present invention or the like can be referred to, for example.

The “identity” as used for the polynucleotide of (ca5) above refers to 80% or more, 85% or more, 90% or more, 95% or more, 96% or more, 97% or more, 98% or more, or 99% or more identity, for example.

The term “one or several” as used for the polynucleotide of (ca6) above regarding substitution and the like refers to 1 to 280, 1 to 210, 1 to 140, 1 to 70, 1 to 63, 1 to 56, 1 to 49, 1 to 35, 1 to 28, 1 to 21, 1 to 14, 1 to 7, 1 to 6, 1 to 5, 1 to 4, 1 to 3, 1 or 2, or 1, for example.

Polynucleotide Coding for L4-18H CAR
(Sequence ID No. 298)
5′-
TCCTATGAGCTGACTCAGCCACCCTCAGTGTCCGTGTCTCCAGGACAGACAGCCAG
CATCACCTGCTCTGGAGATAAATTGGGGGATAAATATGCTTGCTGGTATCAGCAGAA
GCCAGGCCAGTCCCCTGTGCTGGTCATCTATCAAGATAGCAAGCGGCCCTCAGGGA
TCCCTGAGCGATTCTCTGGCTCCAACTCTGGGAACACAGCCACTCTGACCATCAGC
GGGACCCAGGCTATGGATGAGGCTGACTATTACTGTCAGGCGTGGGACAGCAGCAC
ACATGTGGTATTCGGCGGAGGGACCAAGCTGACCGTCCTAGGCTCTACAAGCGGCT
CTGGCAAGCCTGGATCTGGCGAGGGAAGCACCAAGGGCGAAGTGCAGCTTGTGGA
ATCTGGCGGAGGACTGGTGCAGCCTGGAAGAAGCCTGAGACTGTCTTGTGCCGCC
AGCGGCTTCACCTTCGACGATTATGCCATGCACTGGGTCCGACAGGCCCCTGGAAA
AGGCCTTGAATGGGTGTCCGGCATCTCTTGGAACAGCGGCAGAATCGGCTACGCCG
ACTCTGTGAAGGGCAGATTCACCATCAGCCGGGACAACGCCAAGAACAGCCTGTT
CCTGCAGATGAACTCCCTGAGAGCCGAGGACACCGCCGTGTACTACTGTGCCAGAG
ATCAGGGCTACCACTACTACGACTCTGCCGAGCACGCCTTCGATATCTGGGGCCAGG
GAACAGTGGTCACCGTTAGTTCTGCGGCCGCAATCGAAGTGATGTACCCCCCTCCC
TACCTGGACAACGAGAAGTCCAACGGCACCATTATCCACGTGAAGGGAAAGCACC
TGTGCCCCAGCCCTCTGTTCCCTGGCCCTAGCAAGCCTTTCTGGGTGCTGGTGGTCG
TGGGCGGAGTGCTGGCCTGTTATAGCCTGCTCGTGACCGTGGCCTTCATCATCTTTT
GGGTGCGCAGCAAGCGGAGCCGGCTGCTGCACAGCGACTACATGAACATGACCCC
CAGACGGCCCGGACCCACCAGAAAGCACTACCAGCCTTACGCCCCTCCCAGAGAC
TTCGCCGCCTACAGATCTCGAGTGAAGTTCAGCAGAAGCGCCGACGCCCCTGCCTA
TCAGCAGGGCCAGAACCAGCTGTACAACGAGCTGAACCTGGGCAGACGGGAAGA
GTACGACGTGCTGGACAAGCGGAGAGGCAGGGACCCTGAGATGGGCGGCAAGCCC
AGAAGAAAGAACCCCCAGGAAGGCCTGTATAACGAACTGCAGAAAGACAAGATGG
CCGAGGCCTACAGCGAGATCGGCATGAAGGGCGAGCGGAGAAGAGGCAAGGGCC
ACGATGGCCTGTACCAGGGCCTGAGCACCGCCACCAAGGACACCTATGACGCCCTG
CACATGCAGGCCCTGCCCCCCAGA-3′
Polynucleotide Coding for L7-18H CAR
(Sequence ID No. 299)
5′-
CAGTCTGCCCTGACTCAGCCTCGCTCAGTGTCCGGGTCTCCTGGACAGTCAGTCAC
CATCTCCTGCACTGGAACCAGCAGTGATGTTGGTGGTTATAACTATGTCTCCTGGTA
CCAACAACACCCAGGCAAAGCCCCCAAACTCATGATTTATGATGTCAGTAATCGGC
CCTCAGGGGTTTCTAATCGCTTCTCTGGCTCCAAGTCTGGCAACACGGCCTCCCTGA
CCATCTCTGGGCTCCAGACTGGGGACGAGGCCGATTATTACTGCGGAACATGGGAT
ACCAGCCTGACTGCTGTGGTATTCGGCGGAGGGACCGAGCTGACCGTCCTCGGCTC
TACAAGCGGCTCTGGCAAGCCTGGATCTGGCGAGGGAAGCACCAAGGGCGAAGTG
CAGCTTGTGGAATCTGGCGGAGGACTGGTGCAGCCTGGAAGAAGCCTGAGACTGT
CTTGTGCCGCCAGCGGCTTCACCTTCGACGATTATGCCATGCACTGGGTCCGACAG
GCCCCTGGAAAAGGCCTTGAATGGGTGTCCGGCATCTCTTGGAACAGCGGCAGAAT
CGGCTACGCCGACTCTGTGAAGGGCAGATTCACCATCAGCCGGGACAACGCCAAG
AACAGCCTGTTCCTGCAGATGAACTCCCTGAGAGCCGAGGACACCGCCGTGTACTA
CTGTGCCAGAGATCAGGGCTACCACTACTACGACTCTGCCGAGCACGCCTTCGATAT
CTGGGGCCAGGGAACAGTGGTCACCGTTAGTTCTGCGGCCGCAATCGAAGTGATGT
ACCCCCCTCCCTACCTGGACAACGAGAAGTCCAACGGCACCATTATCCACGTGAAG
GGAAAGCACCTGTGCCCCAGCCCTCTGTTCCCTGGCCCTAGCAAGCCTTTCTGGGT
GCTGGTGGTCGTGGGCGGAGTGCTGGCCTGTTATAGCCTGCTCGTGACCGTGGCCT
TCATCATCTTTTGGGTGCGCAGCAAGCGGAGCCGGCTGCTGCACAGCGACTACATG
AACATGACCCCCAGACGGCCCGGACCCACCAGAAAGCACTACCAGCCTTACGCCC
CTCCCAGAGACTTCGCCGCCTACAGATCTCGAGTGAAGTTCAGCAGAAGCGCCGAC
GCCCCTGCCTATCAGCAGGGCCAGAACCAGCTGTACAACGAGCTGAACCTGGGCA
GACGGGAAGAGTACGACGTGCTGGACAAGCGGAGAGGCAGGGACCCTGAGATGG
GCGGCAAGCCCAGAAGAAAGAACCCCCAGGAAGGCCTGTATAACGAACTGCAGAA
AGACAAGATGGCCGAGGCCTACAGCGAGATCGGCATGAAGGGCGAGCGGAGAAG
AGGCAAGGGCCACGATGGCCTGTACCAGGGCCTGAGCACCGCCACCAAGGACACC
TATGACGCCCTGCACATGCAGGCCCTGCCCCCCAGA-3′
Polynucleotide Coding for L9-18H CAR
(Sequence ID No. 300)
5′-
CAGTCTGTGTTGACGCAGCCGCCCTCAGTGTCTGCGGCCCCAGGACAGAAGGTCA
CCATCTCCTGCTCTGGAAGCTGGTCCAACATTGGAGATGATCATGTCTCCTGGTACC
AGCAGTTCCCAGGAGCAGCCCCCAAACTCCTCATTTATGACACTTCTAAGCGACCC
TCACGCGTTGCTGACCGATTCTCTGGCTCCAAGTCTGGCGCGTCAGCCACCCTGGC
CATCACTGGACTCCAGGCTGGGGACGAGGCCGACTATTATTGCGGAACATGGGAAA
GCAGCCTGAGTGGTGTGGTTTTCGGCGGAGGGACCGAGCTGACCGTCCTAGGCTCT
ACAAGCGGCTCTGGCAAGCCTGGATCTGGCGAGGGAAGCACCAAGGGCGAAGTGC
AGCTTGTGGAATCTGGCGGAGGACTGGTGCAGCCTGGAAGAAGCCTGAGACTGTC
TTGTGCCGCCAGCGGCTTCACCTTCGACGATTATGCCATGCACTGGGTCCGACAGG
CCCCTGGAAAAGGCCTTGAATGGGTGTCCGGCATCTCTTGGAACAGCGGCAGAATC
GGCTACGCCGACTCTGTGAAGGGCAGATTCACCATCAGCCGGGACAACGCCAAGA
ACAGCCTGTTCCTGCAGATGAACTCCCTGAGAGCCGAGGACACCGCCGTGTACTAC
TGTGCCAGAGATCAGGGCTACCACTACTACGACTCTGCCGAGCACGCCTTCGATATC
TGGGGCCAGGGAACAGTGGTCACCGTTAGTTCTGCGGCCGCAATCGAAGTGATGTA
CCCCCCTCCCTACCTGGACAACGAGAAGTCCAACGGCACCATTATCCACGTGAAGG
GAAAGCACCTGTGCCCCAGCCCTCTGTTCCCTGGCCCTAGCAAGCCTTTCTGGGTG
CTGGTGGTCGTGGGCGGAGTGCTGGCCTGTTATAGCCTGCTCGTGACCGTGGCCTT
CATCATCTTTTGGGTGCGCAGCAAGCGGAGCCGGCTGCTGCACAGCGACTACATGA
ACATGACCCCCAGACGGCCCGGACCCACCAGAAAGCACTACCAGCCTTACGCCCCT
CCCAGAGACTTCGCCGCCTACAGATCTCGAGTGAAGTTCAGCAGAAGCGCCGACG
CCCCTGCCTATCAGCAGGGCCAGAACCAGCTGTACAACGAGCTGAACCTGGGCAG
ACGGGAAGAGTACGACGTGCTGGACAAGCGGAGAGGCAGGGACCCTGAGATGGG
CGGCAAGCCCAGAAGAAAGAACCCCCAGGAAGGCCTGTATAACGAACTGCAGAAA
GACAAGATGGCCGAGGCCTACAGCGAGATCGGCATGAAGGGCGAGCGGAGAAGA
GGCAAGGGCCACGATGGCCTGTACCAGGGCCTGAGCACCGCCACCAAGGACACCT
ATGACGCCCTGCACATGCAGGCCCTGCCCCCCAGA-3′
Polynucleotide Coding for L13-18H CAR
(Sequence ID No. 301)
5′-
CAGTCTGCCCTGACTCAGCCTGCCTCCGTGTCTGGGTCTCCTGGACAGTCGATCAC
CATCTCCTGCACTGGACTCAGCAGTGACGTTGGTGGTTATGACTATGTCTCCTGGTA
CCAACAACACCCAGGCATAGCCCCCAAACTCATGATTTATGATGTCACTAATCGGCC
CTCAGGGGTTTCTAGTCGCTTCTCTGGCTCCAAGTCTGGCAACACGGCCTCCCTGA
CCATCTCTGGGCTCCAGGCTGAGGACGAGGCTGATTATTACTGCAGCTCATATACAA
CCAGCACGACTTGGGTCTTCGGAGCTGGGACCAAGCTGACCGTCCTAGGCTCTACA
AGCGGCTCTGGCAAGCCTGGATCTGGCGAGGGAAGCACCAAGGGCGAAGTGCAGC
TTGTGGAATCTGGCGGAGGACTGGTGCAGCCTGGAAGAAGCCTGAGACTGTCTTGT
GCCGCCAGCGGCTTCACCTTCGACGATTATGCCATGCACTGGGTCCGACAGGCCCC
TGGAAAAGGCCTTGAATGGGTGTCCGGCATCTCTTGGAACAGCGGCAGAATCGGCT
ACGCCGACTCTGTGAAGGGCAGATTCACCATCAGCCGGGACAACGCCAAGAACAG
CCTGTTCCTGCAGATGAACTCCCTGAGAGCCGAGGACACCGCCGTGTACTACTGTG
CCAGAGATCAGGGCTACCACTACTACGACTCTGCCGAGCACGCCTTCGATATCTGGG
GCCAGGGAACAGTGGTCACCGTTAGTTCTGCGGCCGCAATCGAAGTGATGTACCCC
CCTCCCTACCTGGACAACGAGAAGTCCAACGGCACCATTATCCACGTGAAGGGAAA
GCACCTGTGCCCCAGCCCTCTGTTCCCTGGCCCTAGCAAGCCTTTCTGGGTGCTGGT
GGTCGTGGGCGGAGTGCTGGCCTGTTATAGCCTGCTCGTGACCGTGGCCTTCATCAT
CTTTTGGGTGCGCAGCAAGCGGAGCCGGCTGCTGCACAGCGACTACATGAACATGA
CCCCCAGACGGCCCGGACCCACCAGAAAGCACTACCAGCCTTACGCCCCTCCCAG
AGACTTCGCCGCCTACAGATCTCGAGTGAAGTTCAGCAGAAGCGCCGACGCCCCTG
CCTATCAGCAGGGCCAGAACCAGCTGTACAACGAGCTGAACCTGGGCAGACGGGA
AGAGTACGACGTGCTGGACAAGCGGAGAGGCAGGGACCCTGAGATGGGCGGCAA
GCCCAGAAGAAAGAACCCCCAGGAAGGCCTGTATAACGAACTGCAGAAAGACAA
GATGGCCGAGGCCTACAGCGAGATCGGCATGAAGGGCGAGCGGAGAAGAGGCAA
GGGCCACGATGGCCTGTACCAGGGCCTGAGCACCGCCACCAAGGACACCTATGAC
GCCCTGCACATGCAGGCCCTGCCCCCCAGA-3′
Polynucleotide Coding for L14-18H CAR
(Sequence ID No. 302)
5′-
CAGTCTGCCCTGACTCAGCCTGCCTCCGTGTCTGGGTCTCCTGGACAGTCGATCAC
CATCTCCTGCACTGGAACCACCAGTGACGTTGGTACTACTAATTATGTCTCCTGGTA
CCAGCAACACCCAGGCAAAGCCCCCAAACTCCTAATTTATGATGTCACTAATCGGCC
CTCAGGGGTCCCTGATCGCTTCTCTGGCTCCAAGTCTGCCAACACGGCCTCCCTGA
CCATCTCTGGGCTCCAGGCTGAGGACGAGGCTGATTATTACTGCTGCTCATATGCAG
GCAGCTACACCTTCGTGGTATTCGGCGGAGGGACCGAGCTGACCGTCCTAGGCTCT
ACAAGCGGCTCTGGCAAGCCTGGATCTGGCGAGGGAAGCACCAAGGGCGAAGTGC
AGCTTGTGGAATCTGGCGGAGGACTGGTGCAGCCTGGAAGAAGCCTGAGACTGTC
TTGTGCCGCCAGCGGCTTCACCTTCGACGATTATGCCATGCACTGGGTCCGACAGG
CCCCTGGAAAAGGCCTTGAATGGGTGTCCGGCATCTCTTGGAACAGCGGCAGAATC
GGCTACGCCGACTCTGTGAAGGGCAGATTCACCATCAGCCGGGACAACGCCAAGA
ACAGCCTGTTCCTGCAGATGAACTCCCTGAGAGCCGAGGACACCGCCGTGTACTAC
TGTGCCAGAGATCAGGGCTACCACTACTACGACTCTGCCGAGCACGCCTTCGATATC
TGGGGCCAGGGAACAGTGGTCACCGTTAGTTCTGCGGCCGCAATCGAAGTGATGTA
CCCCCCTCCCTACCTGGACAACGAGAAGTCCAACGGCACCATTATCCACGTGAAGG
GAAAGCACCTGTGCCCCAGCCCTCTGTTCCCTGGCCCTAGCAAGCCTTTCTGGGTG
CTGGTGGTCGTGGGCGGAGTGCTGGCCTGTTATAGCCTGCTCGTGACCGTGGCCTT
CATCATCTTTTGGGTGCGCAGCAAGCGGAGCCGGCTGCTGCACAGCGACTACATGA
ACATGACCCCCAGACGGCCCGGACCCACCAGAAAGCACTACCAGCCTTACGCCCCT
CCCAGAGACTTCGCCGCCTACAGATCTCGAGTGAAGTTCAGCAGAAGCGCCGACG
CCCCTGCCTATCAGCAGGGCCAGAACCAGCTGTACAACGAGCTGAACCTGGGCAG
ACGGGAAGAGTACGACGTGCTGGACAAGCGGAGAGGCAGGGACCCTGAGATGGG
CGGCAAGCCCAGAAGAAAGAACCCCCAGGAAGGCCTGTATAACGAACTGCAGAAA
GACAAGATGGCCGAGGCCTACAGCGAGATCGGCATGAAGGGCGAGCGGAGAAGA
GGCAAGGGCCACGATGGCCTGTACCAGGGCCTGAGCACCGCCACCAAGGACACCT
ATGACGCCCTGCACATGCAGGCCCTGCCCCCCAGA-3′
Polynucleotide Coding for L16-18H CAR
(Sequence ID No. 303)
5′-
CAGTCTGCCCTGACTCAGCCTCCCTCCGCGTCCGGGTCTCCTGGACAGTCAGTCAC
CATCTCCTGCACTGGAACCAGCAGTGACGTTGGTGTTTATAACTATGTCTCCTGGTA
CCAACAACACCCAGGCAAAGCCCCCAAACTCATGATTTATGATGTCAGTAAGCGGC
CCTCAGGGGTCCCTGATCGCTTCTCTGGCTCCAAGTCTGCCAACACGGCCTCCCTG
ACCATCTCTGGGCTCCAGGCTGAGGATGAGGCTGATTATTACTGTGCAGCATGGGAT
GACAGCCTGAATGGTGTGGTATTCGGCGGAGGCACCCAGCTGACCGTCCTCGGCTC
TACAAGCGGCTCTGGCAAGCCTGGATCTGGCGAGGGAAGCACCAAGGGCGAAGTG
CAGCTTGTGGAATCTGGCGGAGGACTGGTGCAGCCTGGAAGAAGCCTGAGACTGT
CTTGTGCCGCCAGCGGCTTCACCTTCGACGATTATGCCATGCACTGGGTCCGACAG
GCCCCTGGAAAAGGCCTTGAATGGGTGTCCGGCATCTCTTGGAACAGCGGCAGAAT
CGGCTACGCCGACTCTGTGAAGGGCAGATTCACCATCAGCCGGGACAACGCCAAG
AACAGCCTGTTCCTGCAGATGAACTCCCTGAGAGCCGAGGACACCGCCGTGTACTA
CTGTGCCAGAGATCAGGGCTACCACTACTACGACTCTGCCGAGCACGCCTTCGATAT
CTGGGGCCAGGGAACAGTGGTCACCGTTAGTTCTGCGGCCGCAATCGAAGTGATGT
ACCCCCCTCCCTACCTGGACAACGAGAAGTCCAACGGCACCATTATCCACGTGAAG
GGAAAGCACCTGTGCCCCAGCCCTCTGTTCCCTGGCCCTAGCAAGCCTTTCTGGGT
GCTGGTGGTCGTGGGCGGAGTGCTGGCCTGTTATAGCCTGCTCGTGACCGTGGCCT
TCATCATCTTTTGGGTGCGCAGCAAGCGGAGCCGGCTGCTGCACAGCGACTACATG
AACATGACCCCCAGACGGCCCGGACCCACCAGAAAGCACTACCAGCCTTACGCCC
CTCCCAGAGACTTCGCCGCCTACAGATCTCGAGTGAAGTTCAGCAGAAGCGCCGAC
GCCCCTGCCTATCAGCAGGGCCAGAACCAGCTGTACAACGAGCTGAACCTGGGCA
GACGGGAAGAGTACGACGTGCTGGACAAGCGGAGAGGCAGGGACCCTGAGATGG
GCGGCAAGCCCAGAAGAAAGAACCCCCAGGAAGGCCTGTATAACGAACTGCAGAA
AGACAAGATGGCCGAGGCCTACAGCGAGATCGGCATGAAGGGCGAGCGGAGAAG
AGGCAAGGGCCACGATGGCCTGTACCAGGGCCTGAGCACCGCCACCAAGGACACC
TATGACGCCCTGCACATGCAGGCCCTGCCCCCCAGA-3′
Polynucleotide Coding for L17-18H CAR
(Sequence ID No. 304)
5′-
CAGTCTGTACTGACTCAACCACCCTCAGCGTCTGGGACCCCCGGGCAGAGGGTCAC
CATCTCTTGCTCTGGCAGCAGCTCCAACATCGGAAATAATTATGTATGCTGGTACCA
ACACCTCCCAGGAACGGCCCCCAAACTTCTCATTTATGACAATGTTAAGCGACCCTC
AGGGATTCCTGACCGATTCTCTGGCTCCAAGTCTGGCACGTCAGCCTCCCTGGCCAT
CAGTGGGCTCCGGTCCGAGGATGAGGCTGATTATTACTGTGCAGCATGGGATGACA
GCCTGAGTGCCATATTCGGCGGAGGGACCGAGCTGACCGTCCTAGGCTCTACAAGC
GGCTCTGGCAAGCCTGGATCTGGCGAGGGAAGCACCAAGGGCGAAGTGCAGCTTG
TGGAATCTGGCGGAGGACTGGTGCAGCCTGGAAGAAGCCTGAGACTGTCTTGTGC
CGCCAGCGGCTTCACCTTCGACGATTATGCCATGCACTGGGTCCGACAGGCCCCTG
GAAAAGGCCTTGAATGGGTGTCCGGCATCTCTTGGAACAGCGGCAGAATCGGCTAC
GCCGACTCTGTGAAGGGCAGATTCACCATCAGCCGGGACAACGCCAAGAACAGCC
TGTTCCTGCAGATGAACTCCCTGAGAGCCGAGGACACCGCCGTGTACTACTGTGCC
AGAGATCAGGGCTACCACTACTACGACTCTGCCGAGCACGCCTTCGATATCTGGGG
CCAGGGAACAGTGGTCACCGTTAGTTCTGCGGCCGCAATCGAAGTGATGTACCCCC
CTCCCTACCTGGACAACGAGAAGTCCAACGGCACCATTATCCACGTGAAGGGAAAG
CACCTGTGCCCCAGCCCTCTGTTCCCTGGCCCTAGCAAGCCTTTCTGGGTGCTGGTG
GTCGTGGGCGGAGTGCTGGCCTGTTATAGCCTGCTCGTGACCGTGGCCTTCATCATC
TTTTGGGTGCGCAGCAAGCGGAGCCGGCTGCTGCACAGCGACTACATGAACATGAC
CCCCAGACGGCCCGGACCCACCAGAAAGCACTACCAGCCTTACGCCCCTCCCAGA
GACTTCGCCGCCTACAGATCTCGAGTGAAGTTCAGCAGAAGCGCCGACGCCCCTGC
CTATCAGCAGGGCCAGAACCAGCTGTACAACGAGCTGAACCTGGGCAGACGGGAA
GAGTACGACGTGCTGGACAAGCGGAGAGGCAGGGACCCTGAGATGGGCGGCAAG
CCCAGAAGAAAGAACCCCCAGGAAGGCCTGTATAACGAACTGCAGAAAGACAAG
ATGGCCGAGGCCTACAGCGAGATCGGCATGAAGGGCGAGCGGAGAAGAGGCAAG
GGCCACGATGGCCTGTACCAGGGCCTGAGCACCGCCACCAAGGACACCTATGACGC
CCTGCACATGCAGGCCCTGCCCCCCAGA-3′
Polynucleotide Coding for L22-18H CAR
(Sequence ID No. 305)
5′-
CAGTCTGCCCTGACTCAGCCTGCCTCCGTGTCTGGGTCTCCTGGACAGTCGATCAC
CATCTCCTGCACTGGAACCAGCAGTGACGTTGGTGGTTATAACTATGTCTCCTGGTA
CCAACAACACCCAGGCAAAGCCCCCAAACTCATGATTTATGATGTCAGTAATCGGC
CCTCAGGGGTCCCTGATCGCTTCTCTGGCTCCAAGTCTGGCACCTCAGCCTCCCTGG
CCATCAGTGGGCTCCAGTCTGAGGATGAGGCTGATTATTACTGCCACTCCTATGACA
GCAGCCTGAGTCATGTCTTCGGAACTGGGACCAAGGTCACCGTCCTAGGCTCTACA
AGCGGCTCTGGCAAGCCTGGATCTGGCGAGGGAAGCACCAAGGGCGAAGTGCAGC
TTGTGGAATCTGGCGGAGGACTGGTGCAGCCTGGAAGAAGCCTGAGACTGTCTTGT
GCCGCCAGCGGCTTCACCTTCGACGATTATGCCATGCACTGGGTCCGACAGGCCCC
TGGAAAAGGCCTTGAATGGGTGTCCGGCATCTCTTGGAACAGCGGCAGAATCGGCT
ACGCCGACTCTGTGAAGGGCAGATTCACCATCAGCCGGGACAACGCCAAGAACAG
CCTGTTCCTGCAGATGAACTCCCTGAGAGCCGAGGACACCGCCGTGTACTACTGTG
CCAGAGATCAGGGCTACCACTACTACGACTCTGCCGAGCACGCCTTCGATATCTGGG
GCCAGGGAACAGTGGTCACCGTTAGTTCTGCGGCCGCAATCGAAGTGATGTACCCC
CCTCCCTACCTGGACAACGAGAAGTCCAACGGCACCATTATCCACGTGAAGGGAAA
GCACCTGTGCCCCAGCCCTCTGTTCCCTGGCCCTAGCAAGCCTTTCTGGGTGCTGGT
GGTCGTGGGCGGAGTGCTGGCCTGTTATAGCCTGCTCGTGACCGTGGCCTTCATCAT
CTTTTGGGTGCGCAGCAAGCGGAGCCGGCTGCTGCACAGCGACTACATGAACATGA
CCCCCAGACGGCCCGGACCCACCAGAAAGCACTACCAGCCTTACGCCCCTCCCAG
AGACTTCGCCGCCTACAGATCTCGAGTGAAGTTCAGCAGAAGCGCCGACGCCCCTG
CCTATCAGCAGGGCCAGAACCAGCTGTACAACGAGCTGAACCTGGGCAGACGGGA
AGAGTACGACGTGCTGGACAAGCGGAGAGGCAGGGACCCTGAGATGGGCGGCAA
GCCCAGAAGAAAGAACCCCCAGGAAGGCCTGTATAACGAACTGCAGAAAGACAA
GATGGCCGAGGCCTACAGCGAGATCGGCATGAAGGGCGAGCGGAGAAGAGGCAA
GGGCCACGATGGCCTGTACCAGGGCCTGAGCACCGCCACCAAGGACACCTATGAC
GCCCTGCACATGCAGGCCCTGCCCCCCAGA-3′
Polynucleotide Coding for K4-18H CAR
(Sequence ID No. 306)
5′-
GAAATTGTGTTGACGCAGTCTCCAGGCACCCTGTCTTTGTCTCCAGGGGAAAGAGC
CACCCTCTCCTGCAGGGCCAGTCAGAGTGTTAGCAGCAACTTAGCCTGGTACCAGC
AGAAACCTGGCCAGGCTCCCAGGCTCCTCATCTATGGTGCATCCACCAGGGCCACT
GGTATCCCAGCCAGGTTCAGTGGCAGTGGGTCTGGGACAGAGTTCACTCTCACCAT
CAGCAGCCTGCAGTCTGAAGATTTTGCAGTTTATTACTGTCAGCAGTATAATAACTG
GCCTCCCTTGTACACTTTTGGCCAGGGGACCAAGCTGGAGATCAAAGGCTCTACAA
GCGGCTCTGGCAAGCCTGGATCTGGCGAGGGAAGCACCAAGGGCGAAGTGCAGCT
TGTGGAATCTGGCGGAGGACTGGTGCAGCCTGGAAGAAGCCTGAGACTGTCTTGT
GCCGCCAGCGGCTTCACCTTCGACGATTATGCCATGCACTGGGTCCGACAGGCCCC
TGGAAAAGGCCTTGAATGGGTGTCCGGCATCTCTTGGAACAGCGGCAGAATCGGCT
ACGCCGACTCTGTGAAGGGCAGATTCACCATCAGCCGGGACAACGCCAAGAACAG
CCTGTTCCTGCAGATGAACTCCCTGAGAGCCGAGGACACCGCCGTGTACTACTGTG
CCAGAGATCAGGGCTACCACTACTACGACTCTGCCGAGCACGCCTTCGATATCTGGG
GCCAGGGAACAGTGGTCACCGTTAGTTCTGCGGCCGCAATCGAAGTGATGTACCCC
CCTCCCTACCTGGACAACGAGAAGTCCAACGGCACCATTATCCACGTGAAGGGAAA
GCACCTGTGCCCCAGCCCTCTGTTCCCTGGCCCTAGCAAGCCTTTCTGGGTGCTGGT
GGTCGTGGGCGGAGTGCTGGCCTGTTATAGCCTGCTCGTGACCGTGGCCTTCATCAT
CTTTTGGGTGCGCAGCAAGCGGAGCCGGCTGCTGCACAGCGACTACATGAACATGA
CCCCCAGACGGCCCGGACCCACCAGAAAGCACTACCAGCCTTACGCCCCTCCCAG
AGACTTCGCCGCCTACAGATCTCGAGTGAAGTTCAGCAGAAGCGCCGACGCCCCTG
CCTATCAGCAGGGCCAGAACCAGCTGTACAACGAGCTGAACCTGGGCAGACGGGA
AGAGTACGACGTGCTGGACAAGCGGAGAGGCAGGGACCCTGAGATGGGCGGCAA
GCCCAGAAGAAAGAACCCCCAGGAAGGCCTGTATAACGAACTGCAGAAAGACAA
GATGGCCGAGGCCTACAGCGAGATCGGCATGAAGGGCGAGCGGAGAAGAGGCAA
GGGCCACGATGGCCTGTACCAGGGCCTGAGCACCGCCACCAAGGACACCTATGAC
GCCCTGCACATGCAGGCCCTGCCCCCCAGA-3′
Polynucleotide Coding for K5-18H CAR
(Sequence ID No. 307)
5′-
GAAATTGTGTTGACGCAGTCTCCAGGCACCCTGTCTTTGTCTCCAGGGGAAAGAGC
CACCCTCTCCTGCAGGGCCAGTCAGAGTGTTAGCAGCTACTTAGCCTGGTACCAAC
AGAAACCTGGCCAGGCTCCCAGGCTCCTCATCTATGATGCATCCAACAGGGCCACT
GGCATCCCAGCCAGGTTCAGTGGCAGTGGGTCTGGGACAGACTTCACTCTCACCAT
CAGCAGCCTAGAGCCTGAAGATTTTGCAACTTACTACTGTCAACAGAGTTACAGTA
CCCTTTTGTACACTTTTGGCCAGGGGACCAAGCTGGAGATCAAAGGCTCTACAAGC
GGCTCTGGCAAGCCTGGATCTGGCGAGGGAAGCACCAAGGGCGAAGTGCAGCTTG
TGGAATCTGGCGGAGGACTGGTGCAGCCTGGAAGAAGCCTGAGACTGTCTTGTGC
CGCCAGCGGCTTCACCTTCGACGATTATGCCATGCACTGGGTCCGACAGGCCCCTG
GAAAAGGCCTTGAATGGGTGTCCGGCATCTCTTGGAACAGCGGCAGAATCGGCTAC
GCCGACTCTGTGAAGGGCAGATTCACCATCAGCCGGGACAACGCCAAGAACAGCC
TGTTCCTGCAGATGAACTCCCTGAGAGCCGAGGACACCGCCGTGTACTACTGTGCC
AGAGATCAGGGCTACCACTACTACGACTCTGCCGAGCACGCCTTCGATATCTGGGG
CCAGGGAACAGTGGTCACCGTTAGTTCTGCGGCCGCAATCGAAGTGATGTACCCCC
CTCCCTACCTGGACAACGAGAAGTCCAACGGCACCATTATCCACGTGAAGGGAAAG
CACCTGTGCCCCAGCCCTCTGTTCCCTGGCCCTAGCAAGCCTTTCTGGGTGCTGGTG
GTCGTGGGCGGAGTGCTGGCCTGTTATAGCCTGCTCGTGACCGTGGCCTTCATCATC
TTTTGGGTGCGCAGCAAGCGGAGCCGGCTGCTGCACAGCGACTACATGAACATGAC
CCCCAGACGGCCCGGACCCACCAGAAAGCACTACCAGCCTTACGCCCCTCCCAGA
GACTTCGCCGCCTACAGATCTCGAGTGAAGTTCAGCAGAAGCGCCGACGCCCCTGC
CTATCAGCAGGGCCAGAACCAGCTGTACAACGAGCTGAACCTGGGCAGACGGGAA
GAGTACGACGTGCTGGACAAGCGGAGAGGCAGGGACCCTGAGATGGGCGGCAAG
CCCAGAAGAAAGAACCCCCAGGAAGGCCTGTATAACGAACTGCAGAAAGACAAG
ATGGCCGAGGCCTACAGCGAGATCGGCATGAAGGGCGAGCGGAGAAGAGGCAAG
GGCCACGATGGCCTGTACCAGGGCCTGAGCACCGCCACCAAGGACACCTATGACGC
CCTGCACATGCAGGCCCTGCCCCCCAGA-3′
Polynucleotide Coding for K6-18H CAR
(Sequence ID No. 308)
5′-
GAAATTGTGTTGACACGGTCTCCAGCCACCCTGTCTTTGTCTCCAGGGGAAAGAGC
CACCCTCTCCTGCAGGGCCAGTCAGAGTGTTAGCAGCTACTTAGCCTGGTACCAAC
AGAAACCTGGCCAGGCTCCCAGGCTCCTCATCTATGATGCCTCCAACAGGGCCACT
GGTATTTCAGCCAGGTTCAGTGGCAGTGGGTCTGGGACAGAGTTCACTCTCACCAT
CAGCAGCCTGCAGTCTGAAGATATTGCAACATATTACTGTCAACAGTATGATAGTCT
CCCACTCACTTTCGGCGGAGGGACCAAGCTGGAGATCAAAGGCTCTACAAGCGGC
TCTGGCAAGCCTGGATCTGGCGAGGGAAGCACCAAGGGCGAAGTGCAGCTTGTGG
AATCTGGCGGAGGACTGGTGCAGCCTGGAAGAAGCCTGAGACTGTCTTGTGCCGC
CAGCGGCTTCACCTTCGACGATTATGCCATGCACTGGGTCCGACAGGCCCCTGGAA
AAGGCCTTGAATGGGTGTCCGGCATCTCTTGGAACAGCGGCAGAATCGGCTACGCC
GACTCTGTGAAGGGCAGATTCACCATCAGCCGGGACAACGCCAAGAACAGCCTGT
TCCTGCAGATGAACTCCCTGAGAGCCGAGGACACCGCCGTGTACTACTGTGCCAGA
GATCAGGGCTACCACTACTACGACTCTGCCGAGCACGCCTTCGATATCTGGGGCCAG
GGAACAGTGGTCACCGTTAGTTCTGCGGCCGCAATCGAAGTGATGTACCCCCCTCC
CTACCTGGACAACGAGAAGTCCAACGGCACCATTATCCACGTGAAGGGAAAGCAC
CTGTGCCCCAGCCCTCTGTTCCCTGGCCCTAGCAAGCCTTTCTGGGTGCTGGTGGTC
GTGGGCGGAGTGCTGGCCTGTTATAGCCTGCTCGTGACCGTGGCCTTCATCATCTTT
TGGGTGCGCAGCAAGCGGAGCCGGCTGCTGCACAGCGACTACATGAACATGACCC
CCAGACGGCCCGGACCCACCAGAAAGCACTACCAGCCTTACGCCCCTCCCAGAGA
CTTCGCCGCCTACAGATCTCGAGTGAAGTTCAGCAGAAGCGCCGACGCCCCTGCCT
ATCAGCAGGGCCAGAACCAGCTGTACAACGAGCTGAACCTGGGCAGACGGGAAGA
GTACGACGTGCTGGACAAGCGGAGAGGCAGGGACCCTGAGATGGGCGGCAAGCCC
AGAAGAAAGAACCCCCAGGAAGGCCTGTATAACGAACTGCAGAAAGACAAGATGG
CCGAGGCCTACAGCGAGATCGGCATGAAGGGCGAGCGGAGAAGAGGCAAGGGCC
ACGATGGCCTGTACCAGGGCCTGAGCACCGCCACCAAGGACACCTATGACGCCCTG
CACATGCAGGCCCTGCCCCCCAGA-3′
Polynucleotide Coding for K9-18H CAR
(Sequence ID No. 309)
5′-
GAAATAGTGTTGACACAGTCTCCAGCCACCCTGTCTTTGTCTCCAGGGGAAAGAGC
CACCGTCTCCTGTAGGCCCAGTCAGACCATTAGTGCCAGTTCCGTAGCCTGGTATCA
GCAGAAAGCTGGCCAGGCTCCACGGCTCCTCATCTATGGTGCATCCAGCAGGGCCA
CTGGCATCCCAGACAGGTTCAGTGGCAGTGGGTCTGGGACAGACTTCACTCTCACC
ATCAGCAGACTGGAGCCTGAAGATTTTGCAGTTTATTACTGTCAGCAATTTAATGAA
TGGCCTCTCACTTTCGGCGGAGGGACCAAGGTGGAAATCAAAGGCTCTACAAGCG
GCTCTGGCAAGCCTGGATCTGGCGAGGGAAGCACCAAGGGCGAAGTGCAGCTTGT
GGAATCTGGCGGAGGACTGGTGCAGCCTGGAAGAAGCCTGAGACTGTCTTGTGCC
GCCAGCGGCTTCACCTTCGACGATTATGCCATGCACTGGGTCCGACAGGCCCCTGG
AAAAGGCCTTGAATGGGTGTCCGGCATCTCTTGGAACAGCGGCAGAATCGGCTACG
CCGACTCTGTGAAGGGCAGATTCACCATCAGCCGGGACAACGCCAAGAACAGCCT
GTTCCTGCAGATGAACTCCCTGAGAGCCGAGGACACCGCCGTGTACTACTGTGCCA
GAGATCAGGGCTACCACTACTACGACTCTGCCGAGCACGCCTTCGATATCTGGGGCC
AGGGAACAGTGGTCACCGTTAGTTCTGCGGCCGCAATCGAAGTGATGTACCCCCCT
CCCTACCTGGACAACGAGAAGTCCAACGGCACCATTATCCACGTGAAGGGAAAGC
ACCTGTGCCCCAGCCCTCTGTTCCCTGGCCCTAGCAAGCCTTTCTGGGTGCTGGTG
GTCGTGGGCGGAGTGCTGGCCTGTTATAGCCTGCTCGTGACCGTGGCCTTCATCATC
TTTTGGGTGCGCAGCAAGCGGAGCCGGCTGCTGCACAGCGACTACATGAACATGAC
CCCCAGACGGCCCGGACCCACCAGAAAGCACTACCAGCCTTACGCCCCTCCCAGA
GACTTCGCCGCCTACAGATCTCGAGTGAAGTTCAGCAGAAGCGCCGACGCCCCTGC
CTATCAGCAGGGCCAGAACCAGCTGTACAACGAGCTGAACCTGGGCAGACGGGAA
GAGTACGACGTGCTGGACAAGCGGAGAGGCAGGGACCCTGAGATGGGCGGCAAG
CCCAGAAGAAAGAACCCCCAGGAAGGCCTGTATAACGAACTGCAGAAAGACAAG
ATGGCCGAGGCCTACAGCGAGATCGGCATGAAGGGCGAGCGGAGAAGAGGCAAG
GGCCACGATGGCCTGTACCAGGGCCTGAGCACCGCCACCAAGGACACCTATGACGC
CCTGCACATGCAGGCCCTGCCCCCCAGA-3′
Polynucleotide Coding for K10-18H CAR
(Sequence ID No. 310)
5′-
GAAATTGTGTTGACACAGTCTCCAGCCACCCTGTCTTTGTCTCCAGGGGAAAGAGC
CACCCTCTCCTGCAGGGCCAGTCAGAGTGTTAGCAGCAACTTAGCCTGGTACCAGC
AAAAACTTGGCCAGGCTCCCAGGCTCCTCATCTATGGTGCATCCAGCAGGGCCACT
GGCATCCCAGACAGGTTCAGTGGCAGTGGGTCTGGGACAGACTTCACTCTCACCAT
CAGCAGACTGGAGCCTGAAGATTTTGCAGTGTATTACTGTCAGCAGTATGGTAGCTC
ACCCGATATATTCACTTTCGGCCCTGGGACCAAAGTGGATATCAAAGGCTCTACAAG
CGGCTCTGGCAAGCCTGGATCTGGCGAGGGAAGCACCAAGGGCGAAGTGCAGCTT
GTGGAATCTGGCGGAGGACTGGTGCAGCCTGGAAGAAGCCTGAGACTGTCTTGTG
CCGCCAGCGGCTTCACCTTCGACGATTATGCCATGCACTGGGTCCGACAGGCCCCT
GGAAAAGGCCTTGAATGGGTGTCCGGCATCTCTTGGAACAGCGGCAGAATCGGCTA
CGCCGACTCTGTGAAGGGCAGATTCACCATCAGCCGGGACAACGCCAAGAACAGC
CTGTTCCTGCAGATGAACTCCCTGAGAGCCGAGGACACCGCCGTGTACTACTGTGC
CAGAGATCAGGGCTACCACTACTACGACTCTGCCGAGCACGCCTTCGATATCTGGG
GCCAGGGAACAGTGGTCACCGTTAGTTCTGCGGCCGCAATCGAAGTGATGTACCCC
CCTCCCTACCTGGACAACGAGAAGTCCAACGGCACCATTATCCACGTGAAGGGAAA
GCACCTGTGCCCCAGCCCTCTGTTCCCTGGCCCTAGCAAGCCTTTCTGGGTGCTGGT
GGTCGTGGGCGGAGTGCTGGCCTGTTATAGCCTGCTCGTGACCGTGGCCTTCATCAT
CTTTTGGGTGCGCAGCAAGCGGAGCCGGCTGCTGCACAGCGACTACATGAACATGA
CCCCCAGACGGCCCGGACCCACCAGAAAGCACTACCAGCCTTACGCCCCTCCCAG
AGACTTCGCCGCCTACAGATCTCGAGTGAAGTTCAGCAGAAGCGCCGACGCCCCTG
CCTATCAGCAGGGCCAGAACCAGCTGTACAACGAGCTGAACCTGGGCAGACGGGA
AGAGTACGACGTGCTGGACAAGCGGAGAGGCAGGGACCCTGAGATGGGCGGCAA
GCCCAGAAGAAAGAACCCCCAGGAAGGCCTGTATAACGAACTGCAGAAAGACAA
GATGGCCGAGGCCTACAGCGAGATCGGCATGAAGGGCGAGCGGAGAAGAGGCAA
GGGCCACGATGGCCTGTACCAGGGCCTGAGCACCGCCACCAAGGACACCTATGAC
GCCCTGCACATGCAGGCCCTGCCCCCCAGA-3′

The nucleic acid of the present invention may be introduced into an expression vector, for example. The descriptions above can be applied to the above-mentioned expression vector, for example.

Regarding the nucleic acid of the present invention, the various polynucleotides above can be manufactured using a known genetic engineering technique and/or a known synthesis technique, for example.

### Cell

As described above, the cell of the present invention includes the chimeric antigen receptor of the present invention. The cell of the present invention is characterized by including the chimeric antigen receptor of the present invention, and there is no particular limitation on the other configurations and conditions. The cell of the present invention can be used to treat cancer, for example. The descriptions of the CAR library, the first screening method, the first antibody or the like, the second antibody or the like, the gene, the expression vector, the transformant, the first chimeric antigen receptor, the second chimeric antigen receptor, the nucleic acid, and the like of the present invention can be applied to the cell of the present invention, for example.

The cell of the present invention may also express a CAR other than the CAR of the present invention. The cell is preferably a T cell. The T cell may be a T cell-like cell, for example.

### Cell-Manufacturing Method

As described above, the cell-manufacturing method of the present invention includes an introduction step of introducing the nucleic acid of the present invention into cells. The cell-manufacturing method of the present invention is characterized in that the nucleic acid of the present invention is introduced into cells in the introduction step, and there is no particular limitation on the other steps and conditions. The cell-manufacturing method of the present invention can be used to manufacture cells capable of treating cancer, for example. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, and the like of the present invention can be applied to the cell-manufacturing method of the present invention, for example.

The cell are preferably a T cell or T cell-like cell.

There is no particular limitation on the nucleic acid-introducing method performed in the introduction step, and the descriptions of the first expression step in the first screening method of the present invention can be applied, for example.

The cell-manufacturing method of the present invention may also include an expression step of expressing a chimeric antigen receptor encoded by the nucleic acid in the cells after the introduction step, for example. The expression step can be performed by culturing the cells that have been subjected to the introduction step, for example.

### Cancer Therapeutic Agent

A cancer therapeutic agent (also referred to as a “therapeutic agent” hereinafter) of the present invention includes the cell, gene, expression vector, or nucleic acid of the present invention. The therapeutic agent of the present invention is characterized by including the cell, gene, expression vector, or nucleic acid of the present invention, and there is no particular limitation on the other configurations and conditions. The therapeutic agent of the present invention can be used to treat cancer, for example. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, the cell-manufacturing method, and the like of the present invention can be applied to the cancer therapeutic agent of the present invention, for example.

In the present invention, cancer to be treated is not particularly limited and can be determined as appropriate in accordance with the type of chimeric antigen receptor to be expressed, or the like, for example. When the cancer therapeutic agent of the present invention is a cell, gene, expression vector, or nucleic acid involving a CAR capable of binding to A2/NY-ESO-1₁₅₇, examples of the cancer to be treated include malignant melanoma, lung cancer, synovial sarcoma, breast cancer, esophageal cancer, ovarian cancer, bladder cancer, neuroblastoma, and myeloma. When the cancer therapeutic agent of the present invention is a cell, gene, expression vector, or nucleic acid involving a CAR capable of binding to CD19, examples of the cancer to be treated include B-cell lymphoma, acute lymphatic leukemia, chronic lymphatic leukemia, and multiple myeloma.

There is no particular limitation on the conditions for administrating the therapeutic agent of the present invention, and the administration form, the administration method, the timing of administration, the dosage amount, and the like can be determined as appropriate in accordance with the type of target cancer, the stage of cancer progression, the age of a patient, and the like, for example. There is no particular limitation on a method for using the therapeutic agent of the present invention, and it is sufficient that the therapeutic agent of the present invention is administered to the administration target, for example.

Examples of the administration target include cells, tissues, and organs. Examples of the administration target include humans and non-human animals other than humans. Examples of the non-human animals include mammals such as mice, rats, dogs, monkeys, rabbits, sheep, horses, and pigs. The administration may be in vivo administration or in vitro administration, for example.

The administration method is not particularly limited and can be determined as appropriate in accordance with the administration target, for example. Examples of the administration method include parenteral administration and oral administration. Examples of the parenteral administration include topical administration, subcutaneous administration, intracutaneous administration, intramuscular administration, intraperitoneal administration, intravenous administration, intralymphatic administration, and intratumoral administration.

There is no particular limitation on the administration form of the therapeutic agent of the present invention, and an example thereof is a liquid agent such as an injection solution (an injection) or a drip infusion solution, or the like.

There is no particular limitation on the amount of the cells blended in the therapeutic agent of the present invention. Also, there is no particular limitation on the administration conditions of the cells. When the administration method is intravenous administration, the administration conditions of the cells are as follows: the dose (total) for an adult male human is 1×10⁸ to 3×10¹⁰ cells, and preferably 1×10⁹ to 1×10¹⁰ cells, for example, and the administration frequency is once a week to once every four weeks, for example. It is preferable that the cells are blended in the therapeutic agent of the present invention in such an amount that achieves a concentration at which the administration conditions shown as an example can be realized.

There is no particular limitation on the amount of the gene, expression vector, or nucleic acid blended in the therapeutic agent of the present invention. Also, there is no particular limitation on the administration conditions of the gene, expression vector, or nucleic acid. When the administration method is subcutaneous injection, intravenous injection, or the like, and is systemic administration to a human, the dose (total) is 5 to 5000 mg or 50 to 500 mg, for example, and the administration frequency is once every two weeks to once every eight weeks, for example.

The therapeutic agent of the present invention may include only one of, or a plurality of, the cell, gene, expression vector, and nucleic acid. Also, the therapeutic agent of the present invention may further include other additives. There is no particular limitation on the blend amounts of the additives as long as the functions of the therapeutic agent of the present invention is not inhibited. There is no particular limitation on the additives, and pharmaceutically acceptable additives are preferable, for example. The types of additives are not particularly limited and can be selected as appropriate in accordance with the type of administration target, for example. Examples of the additives include bases, stabilizers, and preservatives.

### Cancer Treatment Method

A cancer treatment method of the present invention includes an administration step of administering the cell, gene, expression vector, or nucleic acid of the present invention to an administration target. The cancer treatment method of the present invention is characterized by administering the cell, gene, expression vector, or nucleic acid of the present invention to an administration target in the administration step, and there is no particular limitation on the other steps and conditions. The treatment method of the present invention can be used to treat cancer. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, the cell-manufacturing method, the therapeutic agent, and the like of the present invention can be applied to the cancer treatment method of the present invention, for example.

In the administration step, any one of, or a plurality of, the cell, gene, expression vector, and nucleic acid of the present invention may be administered.

### Use of Antibody or the Like, or Cell, Gene, Expression Vector, or Nucleic Acid

The present invention is directed to the antibody of the present invention or the antigen-binding fragment thereof to be used in a cancer treatment method, or the antibody of the present invention or the antigen-binding fragment thereof to be used to treat cancer. Also, the present invention is directed to use of the antibody of the present invention or the antigen-binding fragment to manufacture a cancer treatment medicine. Furthermore, the present invention is directed to the cell, gene, expression vector, or nucleic acid of the present invention to be used in a cancer treatment method, or the cell, gene, expression vector, or nucleic acid of the present invention to be used to treat cancer. Also, the present invention is directed to use of the cell, gene, expression vector, or nucleic acid of the present invention to manufacture a cancer treatment medicine. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, the cell-manufacturing method, the therapeutic agent, the treatment method, and the like of the present invention can be applied to the use of the present invention, for example.

### BsAb Library

A bispecific antibody (BsAb) library of the present invention includes nucleic acids coding for first bispecific antibodies (BsAbs), wherein each of the first BsAbs includes a first antigen-binding domain and a second antigen-binding domain, the first antigen-binding domain includes a first single-chain antibody (scFv) capable of binding to a first target antigen, the second antigen-binding domain includes a second scFv to be screened for the ability to bind to a second target antigen, the second scFv includes a second heavy-chain variable region and a second light-chain variable region, the second heavy-chain variable region and the second light-chain variable region meet Condition 1 or Condition 2 below, and the first target antigen or the second target antigen is an immune cell-activating receptor.

### Condition 1:

The heavy-chain complementarity determining region (CDRH) 1, the CDRH2, and the CDRH3 in the second heavy-chain variable region include the CDRH1, the CDRH2, and the CDRH3 in the heavy-chain variable region of an antibody capable of binding to the second target antigen or an antigen-binding fragment of the antibody, respectively, and
the light-chain complementarity determining region (CDRL) 1, the CDRL2, and the CDRL3 in the second light-chain variable region include the CDRL1, the CDRL2, and the CDRL3 in the light-chain variable region of a first B cell receptor, respectively.

### Condition 2:

The heavy-chain complementarity determining region (CDRH) 1, the CDRH2, and the CDRH3 in the second heavy-chain variable region include the CDRH1, the CDRH2, and the CDRH3 in the heavy-chain variable region of a first B cell receptor, respectively, and
the light-chain complementarity determining region (CDRL) 1, the CDRL2, and the CDRL3 in the second light-chain variable region include the CDRL1, the CDRL2, and the CDRL3 in the light-chain variable region of an antibody capable of binding to the second target antigen or an antigen-binding fragment of the antibody, respectively.

In the BsAb library of the present invention, each of the first BsAbs included in the BsAb library of the present invention includes a first antigen-binding domain and a second antigen-binding domain, the first antigen-binding domain includes a first scFv capable of binding to a first target antigen, the second antigen-binding domain includes a second scFv to be screened for the ability to bind to a second target antigen, the second scFv includes a second heavy-chain variable region and a second light-chain variable region, the second heavy-chain variable region and the second light-chain variable region meet Condition 1 or Condition 2 above, the first target antigen or the second target antigen is an immune cell-activating receptor, and there is no particular limitation on the other configurations and conditions. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, the cell-manufacturing method, the therapeutic agent, the treatment method, and the like of the present invention can be applied to the BsAb library of the present invention, for example.

In the BsAb library of the present invention, the first BsAb is a protein that includes the first antigen-binding domain and the second antigen-binding domain. The first antigen-binding domain and the second antigen-binding domain include a first single-chain antibody (scFv) capable of binding to a first target antigen, and a second scFv to be screened for the ability to bind to a second target antigen, respectively. The second scFv has a structure similar to that of an scFv, for example, and the extracellular domain capable of binding to the antigen is changed to the second antigen-binding domain to be screened for the ability to bind to the second target antigen. Moreover, in the BsAb library of the present invention, the first target antigen or the second target antigen is an immune cell-activating receptor. Accordingly, when the first target antigen is the immune cell-activating receptor, if the first BsAb capable of binding to the second target antigen coexists with an immune cell capable of expressing the second target antigen and the first target antigen (the immune cell-activating receptor), the first BsAb can induce signal via the immune cell-activating receptor of the immune cell, thus making it possible to activate the immune cell (e.g., cause the proliferation of the immune cell, the expression of an activation marker, and the like), for example. That is, with the BsAb library of the present invention, when the first target antigen is the immune cell-activating receptor, a second screening method, which will be described later, can be used to select, from the BsAb group expressed from the BsAb library, a first BsAb capable of activating the immune cell as a BsAb capable of binding to the second target antigen. Accordingly, with the BsAb library of the present invention, scFvs capable of binding to the second target antigen can be screened more easily compared with a method in which hybridomas are used and a method in which phage display is used, for example. Moreover, a first BsAb capable of binding to the second target antigen can activate the immune cell in the second screening method, which will be described later, for example, and thus it can be said that the first BsAb is a BsAb capable of activating the immune cell. Therefore, with the BsAb library of the present invention, BsAbs that can induce cytotoxic activity and the like against cells expressing the second target antigen via the immune cell can be screened, for example.

When the second target antigen is the immune cell-activating receptor, if the first BsAb capable of binding to the second target antigen coexists with an immune cell capable of expressing the first target antigen and the second target antigen (the immune cell-activating receptor), the first BsAb can induce signal via the immune cell-activating receptor of the immune cell, thus making it possible to activate the immune cell (e.g., cause the proliferation of the immune cell, the expression of an activation marker, and the like), for example. That is, with the BsAb library of the present invention, when the second target antigen is the immune cell-activating receptor, the second screening method, which will be described later, can be used to select, from the BsAb group expressed from the BsAb library, a first BsAb capable of activating the immune cell as a BsAb capable of binding to the second target antigen. Accordingly, with the BsAb library of the present invention, scFvs capable of binding to the second target antigen can be screened more easily compared with a method in which hybridomas are used and a method in which phage display is used, for example. Moreover, a first BsAb capable of binding to the second target antigen can activate the immune cell in the second screening method, which will be described later, for example, and thus it can be said that the first BsAb is a BsAb capable of activating the immune cell. Therefore, with the BsAb library of the present invention, BsAbs that can induce, via the immune cell, cytotoxic activity and the like against cells expressing the first target antigen can be screened, for example.

In the BsAb library of the present invention, the second antigen-binding domain includes the second scFv to be screened for the ability to bind to the target antigen. The second scFv is a single-chain polypeptide derived from an antigen capable of binding to an antigen, for example, and has a structure similar to that of an scFv that has an ability to bind to the antigen. The scFv is a polypeptide obtained by coupling the fragments (Fvs) of variable regions of the heavy chain (H chain) and the light chain (L chain) of the antibody capable of binding to the antigen. On the other hand, in the second scFv, either the heavy-chain variable region or the light-chain variable region is a variable region of the antibody or the like capable of binding to the second target antigen, and the other one is a variable region derived from a B cell receptor, namely a variable region that may or may not bind to the second target antigen, as described later, for example. With the BsAb library of the present invention in which the second scFv has such a configuration, heavy-chain variable regions or light-chain variable regions capable of binding to the second target antigen can be screened, for example. Therefore, with the BsAb library of the present invention, scFvs capable of binding to the second target antigen can be screened using a smaller number of, or a smaller number of types of, nucleic acids compared with a phage display technique in which heavy-chain variable regions and light-chain variable regions capable of binding to the second target antigen are screened at a time, for example.

Regarding the BsAb library of the present invention, the term “nucleic acid coding for the first BsAb” refers to a nucleic acid (polynucleotide) coding for the amino acid sequence of the first BsAb, for example, and the descriptions above can be applied.

The term “BsAb” as used in the present invention refers to an antibody in which two antigen-binding sites in the antibody molecule are capable of binding to different target antigens, or an antigen-binding fragment of the antibody, for example. The term “different target antigens” means that the epitopes are different, for example, and the epitopes may be epitopes of different antigens, or different epitopes in one antigen. The structure of a known bispecific antibody can be employed as that of the “BsAb”, for example, and specific examples of the structure to be employed include the structures of a dual-variable-domain antibody (DVD-Ig (trademark)), a diabody, a triabody, a tetrabody, a tandab, a flexibody (a combination of an scFv and a diabody), a tandem scFv (e.g., BiTE (registered trademark) manufactured by Micromet), DART (registered trademark) (manufactured by MacroGenics), Fcab (trademark) or mAb² (trademark) (manufactured by F-star), an Fc engineering antibody (manufactured by Xencor), and DuoBody (registered trademark) (manufactured by Genmab). The tandem scFv is preferable because the library can be easily constructed. The tandem scFv is a protein in which two scFvs are coupled to each other via a linker peptide (inter-Fv linker peptide), for example, and in each of the two scFvs, the heavy-chain variable region and the light-chain variable region are coupled to each other via a linker peptide (Fv linker peptide).

Hereinafter, regarding the first BsAb, a case where the first target antigen is an immune cell-activating receptor and a case where the second target antigen is an immune cell-activating receptor will be described.

### (1) First Target Antigen: Immune Cell-Activating Receptor

The term “immune cell-activating receptor” above refers to a receptor that is expressed in the immune cell and that can activate the immune cell capable of expressing the immune cell-activating receptor due to binding to a ligand or cross-linking between a plurality of immune cell-activating receptors, for example. The immune cell-activating receptor can be determined in accordance with the type of immune cell, for example. When the immune cell is a T cell, a specific example of the immune cell-activating receptor is CD3. Example of the CD3 include CD3γ, CD3δ, CD3ε, CD3ζ, CD3η, and complexes thereof. When the immune cell is an NK cell, an example of the immune cell-activating receptor is an NK cell-activating receptor. Examples of the NK cell-activating receptor include CD94/NKG2C, CD94/NKG2E, and NKG2D/NKG2D. When the immune cell is an NKT cell, examples of the immune cell-activating receptor include CD3 and the NK cell-activating receptor. When the immune cell is a B cell, examples of the immune cell-activating receptor include CD19, CD79α, and CD79β.

When the first target antigen is the above-mentioned immune cell-activating receptor, there is no particular limitation on the second target antigen, and the descriptions of the target antigen in the CAR library can be applied, for example.

In the BsAb library of the present invention, the first scFv includes a first heavy-chain variable region and a first light-chain variable region, for example. The first heavy-chain variable region and the first light-chain variable region have structures similar to those of the heavy-chain variable region and the light-chain variable region in an antibody molecule, respectively. In general, the heavy-chain variable region and the light-chain variable region in an antibody molecule each include three complementarity determining regions (CDRs). The CDRs are also referred to as “hypervariable domains”. The CDRs are regions in which the primary structure is particularly likely to be variable in the variable regions of the heavy chain and the light chain, and the primary structure generally includes three CDRs. In the present invention, the three CDRs in the heavy-chain variable region are referred to as a heavy-chain CDR 1 (CDRH1), a heavy-chain CDR 2 (CDRH2), and a heavy-chain CDR 3 (CDRH3), in this order from the amino terminus (N terminus) of the amino acid sequence of the heavy-chain variable region, and the three CDRs in the light-chain variable region are referred to as a light-chain CDR 1 (CDRL1), a light-chain CDR 2 (CDRL2), and a light-chain CDR 3 (CDRL3), in this order from the amino terminus of the amino acid sequence of the light-chain variable region. These sites are close to one another in the three-dimensional structure and determine the binding specificity for an antigen.

The first heavy-chain variable region includes the CDRH1, the CDRH2, and the CDRH3. The first light-chain variable region includes the CDRL1, the CDRL2, and the CDRL3.

An antibody capable of binding to an immune cell-activating receptor such as CD3 or an antigen-binding fragment of the antibody is used for the CDRH1, the CDRH2, and the CDRH3 in the first heavy-chain variable region and the CDRL1, the CDRL2, and the CDRL3 in the first light-chain variable region. For example, the descriptions of an antibody and an antigen-binding fragment regarding the above-described antibody of the present invention or the like can be applied to specific examples of the antibody or the antigen-binding fragment thereof.

Known antibodies or the like can be used as the antibody or the like capable of binding to the immune cell-activating receptor in accordance with the type of immune cell-activating receptor, for example. In a specific example, when the immune cell-activating receptor is CD3, examples of an antibody against CD38 include an OKT3 antibody, a UCHT1 antibody, an L2K antibody, an HIT3a antibody, a 28F11 antibody, and a 27H5 antibody, for example. When the immune cell-activating receptor is CD19, examples of an antibody against CD19 include an FMC63 antibody and SJ25C1. The antibody or the like capable of binding to the immune cell-activating receptor may be an antibody obtained through immunization with the immune cell-activating receptor, for example. The antibody or the like capable of binding to the immune cell-activating receptor may be an scFv obtained using the second screening method of the present invention, which will be described later, for example. The CDRH1, the CDRH2, and the CDRH3 in the first heavy-chain variable region may respectively be polypeptides that consist of the amino acid sequences of the CDRH1, the CDRH2, and the CDRH3 of the antibody or the like capable of binding to the immune cell-activating receptor, or polypeptides that include the amino acid sequences of the CDRH1, the CDRH2, and the CDRH3 of the antibody or the like capable of binding to the immune cell-activating receptor, for example.

Regions in the first heavy-chain variable region other than the CDRH1, the CDRH2, and the CDRH3, namely framework regions (FRs), may include the FRs in the heavy-chain variable region of the antibody or the like capable of binding to the immune cell-activating receptor, for example. The number of the FRs above in the primary structure is generally four. In the present invention, the four FRs in the heavy-chain variable region are referred to as a heavy-chain FR 1 (FRH1), a heavy-chain FR 2 (FRH2), a heavy-chain FR 3 (FRH3), and a heavy-chain FR 4 (FRH4), in this order from the N terminus of the amino acid sequence of the heavy-chain variable region. It should be noted that the CDRHs and the FRHs are arranged such that the FRH1, the CDRH1, the FRH2, the CDRH2, the FRH3, the CDRH3, and the FRH4 are lined up in this order from the N terminus of the amino acid sequence of the heavy-chain variable region, for example. The FRH1, the FRH2, the FRH3, and the FRH4 in the first heavy-chain variable region may respectively be polypeptides that consist of the amino acid sequences of the FRH1, the FRH2, the FRH3, and the FRH4 of the antibody or the like capable of binding to the immune cell-activating receptor, or polypeptides that include the amino acid sequences of the FRH1, the FRH2, the FRH3, and the FRH4 of the antibody or the like capable of binding to the immune cell-activating receptor, for example. It is preferable that “the antibody or the like capable of binding to the immune cell-activating receptor” in the descriptions of the CDRHs and “the antibody or the like capable of binding to the immune cell-activating receptor” in the descriptions of the FRHs are the same antibody or the like.

The first heavy-chain variable region may include the heavy-chain variable region of the antibody or the like capable of binding to the immune cell-activating receptor, for example. In this case, the first heavy-chain variable region may be a polypeptide that consists of the amino acid sequence of the heavy-chain variable region of the antibody or the like capable of binding to the immune cell-activating receptor, or a polypeptide that includes the amino acid sequence of the heavy-chain variable region of the antibody or the like capable of binding to the immune cell-activating receptor, for example.

The CDRL1, the CDRL2, and the CDRL3 in the first light-chain variable region may respectively be polypeptides that consist of the amino acid sequences of the CDRL1, the CDRL2, and the CDRL3 of the antibody or the like capable of binding to the immune cell-activating receptor, or polypeptides that include the amino acid sequences of the CDRL1, the CDRL2, and the CDRL3 of the antibody or the like capable of binding to the immune cell-activating receptor, for example.

The FRs in the first light-chain variable region may include the FRs in the light-chain variable region of the antibody or the like capable of binding to the immune cell-activating receptor, for example. The number of the FRs above in the primary structure is generally four. In the present invention, the four FRs in the light-chain variable region are referred to as a light-chain FR 1 (FRL1), a light-chain FR 2 (FRL2), a light-chain FR 3 (FRL3), and a light-chain FR 4 (FRL4), in this order from the N terminus of the amino acid sequence of the light-chain variable region. It should be noted that the CDRLs and the FRLs are arranged such that the FRL1, the CDRL1, the FRL2, the CDRL2, the FRL3, the CDRL3, and the FRL4 are lined up in this order from the N terminus of the amino acid sequence of the light-chain variable region, for example. The FRL1, the FRL2, the FRL3, and the FRL4 in the first light-chain variable region may respectively be polypeptides that consist of the amino acid sequences of the FRL1, the FRL2, the FRL3, and the FRL4 of the antibody or the like capable of binding to the immune cell-activating receptor, or polypeptides that include the amino acid sequences of the FRL1, the FRL2, the FRL3, and the FRL4 of the antibody or the like capable of binding to the immune cell-activating receptor, for example. It is preferable that “the antibody or the like capable of binding to the immune cell-activating receptor” in the descriptions of the CDRLs and “the antibody or the like capable of binding to the immune cell-activating receptor” in the descriptions of the FRLs are the same antibody or the like.

The first light-chain variable region may include the light-chain variable region of the antibody or the like capable of binding to the immune cell-activating receptor, for example. In this case, the first light-chain variable region may be a polypeptide that consists of the amino acid sequence of the light-chain variable region of the antibody or the like capable of binding to the immune cell-activating receptor, or a polypeptide that includes the amino acid sequence of the light-chain variable region of the antibody or the like capable of binding to the immune cell-activating receptor, for example.

In the first scFv, the first heavy-chain variable region and the first light-chain variable region are coupled to each other via a first linker peptide (first Fv linker peptide), for example. It is preferable that the first Fv linker peptide does not inhibit the first scFv from binding to the immune cell-activating receptor, for example. The first Fv linker peptide is constituted by 1 to 40, 1 to 18, 1 to 15, 1 to 7, 1 to 3, or 1 or 2 amino acids, for example. The first Fv linker peptide is constituted by amino acids such as glycine and serine, for example, and a specific example thereof is (GGGGS)ₙ. n in this formula is an integer from 1 to 6, for example. The amino acid sequence of the first Fv linker peptide may be the amino acid sequence of the polypeptide represented by Sequence ID No. 1 or 2 above, for example.

In the BsAb library of the present invention, the second scFv includes a second heavy-chain variable region and a second light-chain variable region. The second heavy-chain variable region and the second light-chain variable region have structures similar to those of the heavy-chain variable region and the light-chain variable region in an antibody molecule, respectively, and the descriptions of the first scFv above can be applied to the structures thereof.

The second heavy-chain variable region includes the CDRH1, the CDRH2, and the CDRH3. The second light-chain variable region includes the CDRL1, the CDRL2, and the CDRL3. The second heavy-chain variable region and the second light-chain variable region meet Condition 1 or Condition 2 above.

In Condition 1 above, the CDRH1, the CDRH2, and the CDRH3 of an antibody or the like capable of binding to the second target antigen are used as those in the second heavy-chain variable region, and the CDRL1, the CDRL2, and the CDRL3 in the second light-chain variable region are screened for the ability to bind to the second target antigen. For example, the descriptions of an antibody and an antigen-binding fragment regarding the above-described antibody of the present invention or the like can be applied to specific examples of the antibody or the antigen-binding fragment thereof.

Known antibodies or the like can be used as the antibody or the like capable of binding to the second target antigen in accordance with the second target antigen, for example. As a specific example, a 3M4E5 antibody can be used as an antibody against a complex of HLA-A*02:01 and NY-ESO-1₁₅₇₋₁₆₅, for example. An FMC63 antibody can be used as an antibody against human CD19, for example. The antibody or the like capable of binding to the second target antigen may be an scFv obtained using the second screening method of the present invention, which will be described later, or an antibody obtained through immunization with the second target antigen, for example. The CDRH1, the CDRH2, and the CDRH3 in the second heavy-chain variable region may respectively be polypeptides that consist of the amino acid sequences of the CDRH1, the CDRH2, and the CDRH3 of the antibody or the like capable of binding to the second target antigen, or polypeptides that include the amino acid sequences of the CDRH1, the CDRH2, and the CDRH3 of the antibody or the like capable of binding to the second target antigen, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the antibody or the like capable of binding to the second target antigen, or polypeptides that include the amino acid sequences of the FRH1, the FRH2, the FRH3, and the FRH4 of the antibody or the like capable of binding to the second target antigen, for example. It is preferable that “the antibody or the like capable of binding to the second target antigen” in the descriptions of the CDRHs and “the antibody or the like capable of binding to the second target antigen” in the descriptions of the FRHs are the same antibody or the like.

The second heavy-chain variable region may include the heavy-chain variable region of the antibody or the like capable of binding to the second target antigen, for example. In this case, the second heavy-chain variable region may be a polypeptide that consists of the amino acid sequence of the heavy-chain variable region of the antibody or the like capable of binding to the second target antigen, or a polypeptide that includes the amino acid sequence of the heavy-chain variable region of the antibody or the like capable of binding to the second target antigen, for example.

The second light-chain variable region is encoded by a VJ gene fragment formed through VJ gene recombination of a V gene fragment and a J gene fragment, for example. Therefore, the first B cell receptor may be a B cell receptor that includes a polypeptide encoded by an artificial VJ gene fragment designed as an artificial combination of a V gene fragment and a J gene fragment, for example. B cells in a living organism express a light-chain variable region encoded by a product formed through VJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the BsAb library of the present invention, the first B cell receptor is preferably the light-chain variable region of the B cell receptor derived from isolated B cells for the reason that the BsAb library can be more easily prepared.

The CDRL1, the CDRL2, and the CDRL3 in the second light-chain variable region may respectively be polypeptides that consist of the amino acid sequences of the CDRL1, the CDRL2, and the CDRL3 of the first B cell receptor, or polypeptides that include the amino acid sequences of the CDRL1, the CDRL2, and the CDRL3 of the first B cell receptor, for example.

The FRL1, the FRL2, the FRL3, and the FRL4 in the second light-chain variable region may respectively be polypeptides that consist of the amino acid sequences of the FRL1, the FRL2, the FRL3, and the FRL4 of the first B cell receptor, or polypeptides that include the amino acid sequences of the FRL1, the FRL2, the FRL3, and the FRL4 of the first B cell receptor, for example. It is preferable that “the first B cell receptor” in the descriptions of the CDRLs and “the first B cell receptor” in the descriptions of the FRLs are the same B cell receptor.

The second light-chain variable region may include the light-chain variable region of the first B cell receptor, for example. In this case, the second light-chain variable region may be a polypeptide that consists of the amino acid sequence of the light-chain variable region of the first B cell receptor, or a polypeptide that includes the amino acid sequence of the light-chain variable region of the first B cell receptor, for example.

In the second scFv, the second heavy-chain variable region and the second light-chain variable region are coupled to each other via a second linker peptide (second Fv linker peptide), for example. It is preferable that the second Fv linker peptide does not inhibit the second scFv from binding to the second target antigen, for example. The second Fv linker peptide is constituted by 1 to 40, 1 to 18, 1 to 15, 1 to 7, 1 to 3, or 1 or 2 amino acids, for example. The second Fv linker peptide is constituted by amino acids such as glycine and serine, for example, and a specific example thereof is (GGGGS)ₙ. n in this formula is an integer from 1 to 6, for example. The amino acid sequence of the second Fv linker peptide may be the amino acid sequence of the polypeptide represented by Sequence ID No. 1 or 2 above, for example. The second Fv linker peptide may be the same as or different from the first Fv linker peptide, for example.

Next, in Condition 2 above, the CDRL1, the CDRL2, and the CDRL3 in the second light-chain variable region are derived from the antibody or the like capable of binding to the second target antigen, and the CDRH1, the CDRH2, and the CDRH3 in the second heavy-chain variable region are screened for the ability to bind to the second target antigen.

The second heavy-chain variable region is encoded by a VDJ gene fragment formed through VDJ gene recombination of a V gene fragment, a D gene fragment, and a J gene fragment, for example. Therefore, the first B cell receptor may be a B cell receptor that includes a polypeptide encoded by an artificial VDJ gene fragment designed as an artificial combination of a V gene fragment, a D gene fragment, and a J gene fragment, for example. B cells in a living organism express a heavy-chain variable region encoded by a product formed through VDJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the BsAb library of the present invention, the first B cell receptor is preferably the heavy-chain variable region of the B cell receptor derived from isolated B cells for the reason that the BsAb library can be more easily prepared.

The CDRH1, the CDRH2, and the CDRH3 in the second heavy-chain variable region may respectively be polypeptides that consist of the amino acid sequences of the CDRH1, the CDRH2, and the CDRH3 of the first B cell receptor, or polypeptides that include the amino acid sequences of the CDRH1, the CDRH2, and the CDRH3 of the first B cell receptor, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the first B cell receptor, or polypeptides that include the amino acid sequences of the FRH1, the FRH2, the FRH3, and the FRH4 of the first B cell receptor, for example. It is preferable that “the first B cell receptor” in the descriptions of the CDRHs and “the first B cell receptor” in the descriptions of the FRHs are the same B cell receptor.

The second heavy-chain variable region may include the heavy-chain variable region of the first B cell receptor, for example. In this case, the second heavy-chain variable region may be a polypeptide that consists of the amino acid sequence of the heavy-chain variable region of the first B cell receptor, or a polypeptide that includes the amino acid sequence of the heavy-chain variable region of the first B cell receptor, for example.

Regarding the second light-chain variable region, the descriptions of the antibody or the like capable of binding to the second target antigen in Condition 1 can be applied to the above-mentioned antibody or the like capable of binding to the second target antigen, for example. The CDRL1, the CDRL2, and the CDRL3 in the second light-chain variable region may respectively be polypeptides that consist of the amino acid sequences of the CDRL1, the CDRL2, and the CDRL3 of the antibody or the like capable of binding to the second target antigen, or polypeptides that include the amino acid sequences of the CDRL1, the CDRL2, and the CDRL3 of the antibody or the like capable of binding to the second target antigen, for example.

The FRs in the second light-chain variable region may include the FRs in the light-chain variable region of the antibody or the like capable of binding to the second target antigen, for example. The FRL1, the FRL2, the FRL3, and the FRL4 in the second light-chain variable region may respectively be polypeptides that consist of the amino acid sequences of the FRL1, the FRL2, the FRL3, and the FRL4 of the antibody or the like capable of binding to the second target antigen, or polypeptides that include the amino acid sequences of the FRL1, the FRL2, the FRL3, and the FRL4 of the antibody or the like capable of binding to the second target antigen, for example. It is preferable that “the antibody or the like capable of binding to the second target antigen” in the descriptions of the CDRLs and “the antibody or the like capable of binding to the second target antigen” in the descriptions of the FRLs are the same antibody or the like.

The second light-chain variable region may include the light-chain variable region of the antibody or the like capable of binding to the second target antigen, for example. In this case, the second light-chain variable region may be a polypeptide that consists of the amino acid sequence of the light-chain variable region of the antibody or the like capable of binding to the second target antigen, or a polypeptide that includes the amino acid sequence of the light-chain variable region of the antibody or the like capable of binding to the second target antigen, for example.

In the second scFv, the second heavy-chain variable region and the second light-chain variable region are coupled to each other via a second linker peptide (second Fv linker peptide), for example. It is preferable that the second Fv linker peptide does not inhibit the second scFv from binding to the second target antigen, for example. The descriptions of the first Fv linker peptide can be applied to the descriptions of the second Fv linker peptide, for example. The second Fv linker peptide may be the same as or different from the first Fv linker peptide, for example.

The first scFv and the second scFv are directly or indirectly coupled to each other, for example. The coupling between the first scFv and the second scFv can be designed as appropriate in accordance with the type of first BsAb, for example. In a specific example, when the first BsAb is a tandem scFv, the first scFv and the second scFv are coupled to each other via a linker peptide (inter-Fv linker peptide). The inter-Fv linker peptide is constituted by 1 to 40, 1 to 18, 1 to 15, 1 to 7, 1 to 3, or 1 or 2 amino acids, for example. The inter-Fv linker peptide is constituted by amino acids such as glycine and serine, for example, and a specific example thereof is (GGGGS)ₙ. n in this formula is an integer from 1 to 6, for example. The amino acid sequence of the inter-Fv linker peptide may be the amino acid sequence of the polypeptide represented by Sequence ID No. 1 or 2 above, for example.

### (2) Second Target Antigen: Immune Cell-Activating Receptor

There is no particular limitation on the first target antigen, and the descriptions of the target antigen in the CAR library can be applied, for example.

The descriptions of the first target antigen of the BsAb library of the present invention in “(1) First Target Antigen: Immune Cell-Activating Receptor” can be applied to the second target antigen.

In the BsAb library of the present invention, the first scFv includes a first heavy-chain variable region and a first light-chain variable region, for example. The first heavy-chain variable region and the first light-chain variable region have structures similar to those of the heavy-chain variable region and the light-chain variable region in an antibody molecule, respectively, and the descriptions of the first scFv of the BsAb library of the present invention in “(1) First Target Antigen: Immune Cell-Activating Receptor” can be applied. The first heavy-chain variable region includes the CDRH1, the CDRH2, and the CDRH3, for example. The first light-chain variable region includes the CDRL1, the CDRL2, and the CDRL3, for example.

An antibody capable of binding to the first target antigen or an antigen-binding fragment of the antibody is used for the CDRH1, the CDRH2, and the CDRH3 in the first heavy-chain variable region and the CDRL1, the CDRL2, and the CDRL3 in the first light-chain variable region. For example, the descriptions of an antibody and an antigen-binding fragment regarding the above-described antibody of the present invention or the like can be applied to specific examples of the antibody or the antigen-binding fragment thereof.

Known antibodies or the like capable of binding to the first target antigen can be used as the antibody or the like capable of binding to the first target antigen in accordance with the type of first target antigen, for example. The antibody or the like capable of binding to the first target antigen may be an antibody obtained through immunization with the first target antigen, for example. The antibody or the like capable of binding to the first target antigen may be an scFv obtained using the second screening method of the present invention, which will be described later, for example. The CDRH1, the CDRH2, and the CDRH3 in the first heavy-chain variable region may respectively be polypeptides that consist of the amino acid sequences of the CDRH1, the CDRH2, and the CDRH3 of the antibody or the like capable of binding to the first target antigen, or polypeptides that include the amino acid sequences of the CDRH1, the CDRH2, and the CDRH3 of the antibody or the like capable of binding to the first target antigen, for example.

The FRH1, the FRH2, the FRH3, and the FRH4 in the first heavy-chain variable region may respectively be polypeptides that consist of the amino acid sequences of the FRH1, the FRH2, the FRH3, and the FRH4 of the antibody or the like capable of binding to the first target antigen, or polypeptides that include the amino acid sequences of the FRH1, the FRH2, the FRH3, and the FRH4 of the antibody or the like capable of binding to the first target antigen, for example. It is preferable that “the antibody or the like capable of binding to the first target antigen” in the descriptions of the CDRHs and “the antibody or the like capable of binding to the first target antigen” in the descriptions of the FRHs are the same antibody or the like.

The first heavy-chain variable region may include the heavy-chain variable region of the antibody or the like capable of binding to the first target antigen, for example. In this case, the first heavy-chain variable region may be a polypeptide that consists of the amino acid sequence of the heavy-chain variable region of the antibody or the like capable of binding to the first target antigen, or a polypeptide that includes the amino acid sequence of the heavy-chain variable region of the antibody or the like capable of binding to the first target antigen, for example.

The CDRL1, the CDRL2, and the CDRL3 in the first light-chain variable region may respectively be polypeptides that consist of the amino acid sequences of the CDRL1, the CDRL2, and the CDRL3 of the antibody or the like capable of binding to the first target antigen, or polypeptides that include the amino acid sequences of the CDRL1, the CDRL2, and the CDRL3 of the antibody or the like capable of binding to the first target antigen, for example.

The FRL1, the FRL2, the FRL3, and the FRL4 in the first light-chain variable region may respectively be polypeptides that consist of the amino acid sequences of the FRL1, the FRL2, the FRL3, and the FRL4 of the antibody or the like capable of binding to the first target antigen, or polypeptides that include the amino acid sequences of the FRL1, the FRL2, the FRL3, and the FRL4 of the antibody or the like capable of binding to the first target antigen, for example. It is preferable that “the antibody or the like capable of binding to the first target antigen” in the descriptions of the CDRLs and “the antibody or the like capable of binding to the first target antigen” in the descriptions of the FRLs are the same antibody or the like.

The first light-chain variable region may include the light-chain variable region of the antibody or the like capable of binding to the first target antigen, for example. In this case, the first light-chain variable region may be a polypeptide that consists of the amino acid sequence of the light-chain variable region of the antibody or the like capable of binding to the first target antigen, or a polypeptide that includes the amino acid sequence of the light-chain variable region of the antibody or the like capable of binding to the first target antigen, for example.

In the first scFv, the first heavy-chain variable region and the first light-chain variable region are coupled to each other via a first linker peptide (first Fv linker peptide), for example. It is preferable that the first Fv linker peptide does not inhibit the first scFv from binding to the first target antigen, for example. The first Fv linker peptide is constituted by 1 to 40, 1 to 18, 1 to 15, 1 to 7, 1 to 3, or 1 or 2 amino acids, for example. The first Fv linker peptide is constituted by amino acids such as glycine and serine, for example, and a specific example thereof is (GGGGS)ₙ. n in this formula is an integer from 1 to 6, for example. The amino acid sequence of the first Fv linker peptide may be the amino acid sequence of the polypeptide represented by Sequence ID No. 1 or 2 above, for example.

In the BsAb library of the present invention, the second scFv includes a second heavy-chain variable region and a second light-chain variable region. The second heavy-chain variable region and the second light-chain variable region have structures similar to those of the heavy-chain variable region and the light-chain variable region in an antibody molecule, respectively, and the descriptions of the first scFv in (1) above can be applied to the structures.

The second heavy-chain variable region includes the CDRH1, the CDRH2, and the CDRH3. The second light-chain variable region includes the CDRL1, the CDRL2, and the CDRL3. The second heavy-chain variable region and the second light-chain variable region meet Condition 1 or Condition 2 above.

In Condition 1 above, an antibody or the like capable of binding to the immune cell-activating receptor is used for the CDRH1, the CDRH2, and the CDRH3 in the second heavy-chain variable region, and the CDRL1, the CDRL2, and the CDRL3 in the second light-chain variable region are screened for the ability to bind to the immune cell-activating receptor. For example, the descriptions of an antibody and an antigen-binding fragment regarding the above-described antibody of the present invention or the like can be applied to specific examples of the antibody or the antigen-binding fragment thereof.

The descriptions of the antibody or the like capable of binding to the immune cell-activating receptor of the BsAb library of the present invention in “(1) First Target Antigen: Immune Cell-Activating Receptor” can be applied to the above-mentioned antibody or the like capable of binding to the immune cell-activating receptor. The CDRH1, the CDRH2, and the CDRH3 in the second heavy-chain variable region may respectively be polypeptides that consist of the amino acid sequences of the CDRH1, the CDRH2, and the CDRH3 of the antibody or the like capable of binding to the immune cell-activating receptor, or polypeptides that include the amino acid sequences of the CDRH1, the CDRH2, and the CDRH3 of the antibody or the like capable of binding to the immune cell-activating receptor, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the antibody or the like capable of binding to the immune cell-activating receptor, or polypeptides that include the amino acid sequences of the FRH1, the FRH2, the FRH3, and the FRH4 of the antibody or the like capable of binding to the immune cell-activating receptor, for example. It is preferable that “the antibody or the like capable of binding to the immune cell-activating receptor” in the descriptions of the CDRHs and “the antibody or the like capable of binding to the immune cell-activating receptor” in the descriptions of the FRHs are the same antibody or the like.

The second heavy-chain variable region may include the heavy-chain variable region of the antibody or the like capable of binding to the immune cell-activating receptor, for example. In this case, the second heavy-chain variable region may be a polypeptide that consists of the amino acid sequence of the heavy-chain variable region of the antibody or the like capable of binding to the immune cell-activating receptor, or a polypeptide that includes the amino acid sequence of the heavy-chain variable region of the antibody or the like capable of binding to the immune cell-activating receptor, for example.

The second light-chain variable region is encoded by a VJ gene fragment formed through VJ gene recombination of a V gene fragment and a J gene fragment, for example. Therefore, the first B cell receptor may be a B cell receptor that includes a polypeptide encoded by an artificial VJ gene fragment designed as an artificial combination of a V gene fragment and a J gene fragment, for example. B cells in a living organism express a light-chain variable region encoded by a product formed through VJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the BsAb library of the present invention, the first B cell receptor is preferably the light-chain variable region of the B cell receptor derived from isolated B cells for the reason that the BsAb library can be more easily prepared.

The CDRL1, the CDRL2, and the CDRL3 in the second light-chain variable region may respectively be polypeptides that consist of the amino acid sequences of the CDRL1, the CDRL2, and the CDRL3 of the first B cell receptor, or polypeptides that include the amino acid sequences of the CDRL1, the CDRL2, and the CDRL3 of the first B cell receptor, for example.

The FRL1, the FRL2, the FRL3, and the FRL4 in the second light-chain variable region may respectively be polypeptides that consist of the amino acid sequences of the FRL1, the FRL2, the FRL3, and the FRL4 of the first B cell receptor, or polypeptides that include the amino acid sequences of the FRL1, the FRL2, the FRL3, and the FRL4 of the first B cell receptor, for example. It is preferable that “the first B cell receptor” in the descriptions of the CDRLs and “the first B cell receptor” in the descriptions of the FRLs are the same B cell receptor.

The second light-chain variable region may include the light-chain variable region of the first B cell receptor, for example. In this case, the second light-chain variable region may be a polypeptide that consists of the amino acid sequence of the light-chain variable region of the first B cell receptor, or a polypeptide that includes the amino acid sequence of the light-chain variable region of the first B cell receptor, for example.

In the first scFv, the first heavy-chain variable region and the first light-chain variable region are coupled to each other via a first linker peptide (first Fv linker peptide), for example. It is preferable that the first Fv linker peptide does not inhibit the first scFv from binding to the first target antigen, for example. The first Fv linker peptide is constituted by 1 to 40, 1 to 18, 1 to 15, 1 to 7, 1 to 3, or 1 or 2 amino acids, for example. The first Fv linker peptide is constituted by amino acids such as glycine and serine, for example, and a specific example thereof is (GGGGS)ₙ. n in this formula is an integer from 1 to 6, for example. The amino acid sequence of the first Fv linker peptide may be the amino acid sequence of the polypeptide represented by Sequence ID No. 1 or 2 above, for example.

Next, in Condition 2 above, the CDRL1, the CDRL2, and the CDRL3 in the second light-chain variable region are derived from the antibody or the like capable of binding to the immune cell-activating receptor, and the CDRH1, the CDRH2, and the CDRH3 in the second heavy-chain variable region are screened for the ability to bind to the immune cell-activating receptor.

The second heavy-chain variable region is encoded by a VDJ gene fragment formed through VDJ gene recombination of a V gene fragment, a D gene fragment, and a J gene fragment, for example. Therefore, the first B cell receptor may be a B cell receptor that includes a polypeptide encoded by an artificial VDJ gene fragment designed as an artificial combination of a V gene fragment, a D gene fragment, and a J gene fragment, for example. B cells in a living organism express a heavy-chain variable region encoded by a product formed through VDJ gene recombination, for example. Therefore, the first B cell receptor may be a B cell receptor derived from isolated B cells, for example. In this case, the first B cell receptor may be a B cell receptor of B cells derived from a human, for example, and is preferably a B cell receptor derived from human peripheral blood B cells. In the BsAb library of the present invention, the first B cell receptor is preferably the heavy-chain variable region of the B cell receptor derived from isolated B cells for the reason that the BsAb library can be more easily prepared.

The CDRH1, the CDRH2, and the CDRH3 in the second heavy-chain variable region may respectively be polypeptides that consist of the amino acid sequences of the CDRH1, the CDRH2, and the CDRH3 of the first B cell receptor, or polypeptides that include the amino acid sequences of the CDRH1, the CDRH2, and the CDRH3 of the first B cell receptor, for example.

The FRH1, the FRH2, the FRH3, and the FRH4 in the second heavy-chain variable region may respectively be polypeptides that consist of the amino acid sequences of the FRH1, the FRH2, the FRH3, and the FRH4 of the first B cell receptor, or polypeptides that include the amino acid sequences of the FRH1, the FRH2, the FRH3, and the FRH4 of the first B cell receptor, for example. It is preferable that “the first B cell receptor” in the descriptions of the CDRHs and “the first B cell receptor” in the descriptions of the FRHs are the same B cell receptor.

The second heavy-chain variable region may include the heavy-chain variable region of the first B cell receptor, for example. In this case, the second heavy-chain variable region may be a polypeptide that consists of the amino acid sequence of the heavy-chain variable region of the first B cell receptor, or a polypeptide that includes the amino acid sequence of the heavy-chain variable region of the first B cell receptor, for example.

Regarding the second light-chain variable region, the descriptions of the antibody or the like capable of binding to the immune cell-activating receptor of the BsAb library of the present invention in “(1) First Target Antigen: Immune Cell-Activating Receptor” can be applied to the above-mentioned antibody or the like capable of binding to the immune cell-activating receptor, for example. The CDRL1, the CDRL2, and the CDRL3 in the second light-chain variable region may respectively be polypeptides that consist of the amino acid sequences of the CDRL1, the CDRL2, and the CDRL3 of the antibody or the like capable of binding to the immune cell-activating receptor, or polypeptides that include the amino acid sequences of the CDRL1, the CDRL2, and the CDRL3 of the antibody or the like capable of binding to the immune cell-activating receptor, for example.

The FRs in the second light-chain variable region may include the FRs in the light-chain variable region of the antibody or the like capable of binding to the immune cell-activating receptor, for example. The FRL1, the FRL2, the FRL3, and the FRL4 in the second light-chain variable region may respectively be polypeptides that consist of the amino acid sequences of the FRL1, the FRL2, the FRL3, and the FRL4 of the antibody or the like capable of binding to the immune cell-activating receptor, or polypeptides that include the amino acid sequences of the FRL1, the FRL2, the FRL3, and the FRL4 of the antibody or the like capable of binding to the immune cell-activating receptor, for example. It is preferable that “the antibody or the like capable of binding to the immune cell-activating receptor” in the descriptions of the CDRLs and “the antibody or the like capable of binding to the immune cell-activating receptor” in the descriptions of the FRLs are the same antibody or the like.

The second light-chain variable region may include the light-chain variable region of the antibody or the like capable of binding to the immune cell-activating receptor, for example. In this case, the second light-chain variable region may be a polypeptide that consists of the amino acid sequence of the light-chain variable region of the antibody or the like capable of binding to the immune cell-activating receptor, or a polypeptide that includes the amino acid sequence of the light-chain variable region of the antibody or the like capable of binding to the immune cell-activating receptor, for example.

In the second scFv, the second heavy-chain variable region and the second light-chain variable region are coupled to each other via a second linker peptide (second Fv linker peptide), for example. It is preferable that the second Fv linker peptide does not inhibit the second scFv from binding to the immune cell-activating receptor, for example. The descriptions of the first Fv linker peptide can be applied to the descriptions of the second Fv linker peptide, for example. The second Fv linker peptide may be the same as or different from the first Fv linker peptide, for example.

The first scFv and the second scFv may be directly or indirectly coupled to each other, or be formed such that, when the first scFv and the second scFv coexist, the third scFv and the fourth scFv are associated with (bind to) each other, for example. The coupling between the first scFv and the second scFv can be designed as appropriate in accordance with the type of first BsAb, for example. In a specific example, when the first BsAb is a tandem scFv, the first scFv and the second scFv are coupled to each other via a linker peptide (inter-Fv linker peptide). The inter-Fv linker peptide is constituted by 1 to 40, 1 to 18, 1 to 15, 1 to 7, 1 to 3, or 1 or 2 amino acids, for example. The inter-Fv linker peptide is constituted by amino acids such as glycine and serine, for example, and a specific example thereof is (GGGGS)ₙ. n in this formula is an integer from 1 to 6, for example. In a specific example, the amino acid sequence of the inter-Fv linker peptide may be the amino acid sequence of the polypeptide represented by Sequence ID No. 1 or 2 above, for example. The electric charges of the amino acid side chains can be used for the above-mentioned association, for example. In a specific example, the association can be induced by adding a domain that includes positively charged amino acids to one binding domain and adding a domain that includes negatively charged amino acids to the other binding domain, for example. Moreover, a combination of a tag sequence and a polypeptide capable of recognizing the tag sequence, or the like can also be used for the association, for example.

Regarding the arrangement order of the first scFv and the second scFv of each of the first BsAbs in the BsAb library of the present invention, the first scFv and the second scFv may be arranged in this order from the N-terminal side, or the second scFv and the first scFv may be arranged in this order from the N-terminal side. The latter case is preferable because the first BsAb library can be prepared more easily.

An example of the first BsAb is a polypeptide consisting of the amino acid sequence represented by Formula (3) (Sequence ID No. 158) below. Examples of the combination of V₁, V₂, V₃, and V₄ in Formula (3) below include combinations (V1) to (V8) below. When the second scFv meets Condition 1 above, the combination of V₁, V₂, V₃, and V₄ is preferably the combination (V7) or (V8) below. When the second scFv meets Condition 2 above, the combination of V₁, V₂, V₃, and V₄ is preferably the combination (V5) or (V6) below. In Formula (3) below, the amino acid sequence between V₁ and V₂ is the amino acid sequence of the first Fv linker peptide, the amino acid sequence between V₂ and V₃ is the amino acid sequence of the inter-Fv linker peptide, and the amino acid sequence between V₃ and V₄ is the amino acid sequence of the second Fv linker peptide. In Formula (3) below, the first Fv linker peptide and the second Fv linker peptide are Fv linker peptides 1, but may also be independently an Fv linker peptide 2.

[V₁]-[GSTSGSGKPGSGEGSTKG]-[V₂]-[SGSG]-[V₃]-
[GSTSGSGKPGSGEGSTKG]-[V₄]......(3)

(V1) V₁: V_{H1}, V₂: V_{L1}, V₃: V_{H2}, V₄: V_{L2}
(V2) V₁: V_{L1}, V₂: V_{H1}, V₃: V_{H2}, V₄: V_{L2}
(V3) V₁: V_{H1}, V₂: V_{L1}, V₃: V_{L2}, V₄: V_{H2}
(V4) V₁: V_{L1}, V₂: V_{H1}, V₃: V_{L2}, V₄: V_{H2}
(V5) V₁: V_{H2}, V₂: V_{L2}, V₃: V_{H1}, V₅: V_{L1}
(V6) V₁: V_{H2}, V₂: V_{L2}, V₃: V_{L1}, V₄: V_{H1}
(V7) V₁: V_{L2}, V₂: V_{H2}, V₃: V_{H1}, V₄: V_{L1}
(V8) V₁: V_{L2}, V₂: V_{H2}, V₃: V_{L1}, V₄: V_{H1}
   V_{H1}: first heavy-chain variable region
   V_{L1}: first light-chain variable region
   V_{H2}: second heavy-chain variable region
   V_{L2}: second light-chain variable region

An example of the nucleic acid coding for the first BsAb is a polynucleotide consisting of the base sequence represented by Formula (4) (Sequence ID No. 159) below. Examples of the combination of N₁, N₂, N₃, and N₄ in Formula (4) below include combinations (N1) to (N8) below. The combinations (N1) to (N8) below are nucleic acids coding for the first BsAbs of the combinations (V1) to (V8) above, respectively. In Formula (4) below, the base sequence between N₁ and N₂ is the base sequence coding for the first Fv linker peptide, the base sequence between N₂ and N₃ is the base sequence coding for the inter-Fv linker peptide, and the base sequence between N₃ and N₄ is the base sequence coding for the second Fv linker peptide. In Formula (4) below, the base sequences coding for the first Fv linker peptide and the second Fv linker peptide are base sequences coding for Fv linker peptides 1, but may also be independently a base sequence coding for an Fv linker peptide 2.

5′-[N₁]-[GGCTCTACAAGCGGCTCTGGCAAGCCTGGATCTGGCGAG
GGAAGCACCAAGGGC]-[N₂]-[AGCGGATCTGGC]-[N₃]-[GGCTC
TACAAGCGGCTCTGGCAAGCCTGGATCTGGCGAGGGAAGCACCAAGG
GC]-[N₄]-3′......(4)

(N1) N₁: N_{H1}, N₂: N_{L1}, N₃: N_{H2}, N₄: N_{L2}
(N2) N₁: N_{L1}, N₂: N_{H1}, N₃: N_{H2}, N₄: N_{L2}
(N3) N₁: N_{H1}, N₂: N_{L1}, N₃: N_{L2}, N₄: N_{H2}
(N4) N₁: N_{L1}, N₂: N_{H1}, N₃: N_{L2}, N₄: N_{H2}
(N5) N₁: N_{H2}, N₂: N_{L2}, N₃: N_{H1}, N₄: N_{L1}
(N6) N₁: N_{H2}, N₂: N_{L2}, N₃: N_{L1}, N₄: N_{H1}
(N7) N₁: N_{L2}, N₂: N_{H2}, N₃: N_{H1}, N₄: N_{L1}
(N8) N₁: N_{L2}, N₂: N_{H2}, N₃: N_{L1}, N₄: N_{H1}
   N_{H1}: base sequence coding for first heavy-chain variable region
   N_{L1}: base sequence coding for first light-chain variable region
   N_{H2}: base sequence coding for second heavy-chain variable region
   N_{L2}: base sequence coding for second light-chain variable region

It is preferable that the BsAb library of the present invention includes a plurality of types of nucleic acids, for example. In this case, the BsAb library of the present invention is a mixture of a plurality of types of nucleic acids, for example. It is preferable that some or all of the plurality of types of nucleic acids code for different first BsAbs, and preferably code for different second antigen-binding domains, for example. When the plurality of types of nucleic acids code for different second antigen-binding domains, the regions other than the second antigen-binding domains in the first BsAbs have the same amino acid sequence or different amino acid sequences, for example. The number of types of nucleic acids included in the BsAb library of the present invention is 1×10⁵ to 1×10⁶ or 1×10⁶ to 5×10⁶, for example, and preferably about 1×10⁶ (e.g., 8×10⁵ to 2×10⁶). In the phage display technique, the number of types of nucleic acids required to screen scFv capable of binding to the target antigen is about 1×10⁸. On the other hand, with the BsAb library of the present invention, scFvs capable of binding to the second target antigen can be screened using as few as about 1×10⁶ types of nucleic acids, for example. Accordingly, with the BsAb library of the present invention, scFvs capable of binding to the second target antigen can be screened using a smaller number of, or a smaller number of types of, nucleic acids compared with the phage display technique, for example.

The first BsAb may include a signal peptide at the N terminus, for example. An example of the signal peptide is a signal peptide serving as an endoplasmic reticulum transport signal. The first BsAb may include a tag, for example. Examples of the tag include a peptide tag and a protein tag. Examples of the tag include a FLAG (registered trademark) tag, a HA tag, a His tag, a Myc tag, a V5 tag, and a truncated NGFR (nerve growth factor receptor). The tag peptide is added to at least either of the N terminus or the C terminus of the first BsAb, for example. When the truncated NGFR is used as the tag, the tag is arranged at the C-terminal side of the first BsAb. Thus, with the BsAb library of the present invention, the nucleic acid introduction efficiency can be adjusted such that one type of nucleic acid coding for a first BsAb is introduced per cell, for example. The first BsAb and the tag may be coupled to each other via a linker peptide.

In the present invention, the nucleic acids coding for the first BsAbs can be prepared based on the amino acid sequences of the first BsAbs using an ordinary method, for example. In a specific example, the nucleic acids coding for the first BsAbs can be prepared based on the base sequences coding for the amino acid sequences obtained from the database in which the amino acid sequences of the above-described domains are registered, using a molecular biological technique and/or a chemical synthesis method, for example. The base sequences of the nucleic acids may be subjected to codon optimization in accordance with the source of cells in which the BsAb library of the present invention is to be expressed, for example.

The nucleic acids coding for the first BsAbs may be introduced into expression vectors, for example. The expression vectors can be prepared by coupling the nucleic acids coding for the first BsAbs to linking vectors, for example. There is no particular limitation on the types of linking vectors, and examples thereof include: retroviral vectors such as oncoretroviral vectors, lentiviral vectors, and pseudo type vectors; and viral vectors such as adenoviral vectors, adeno-associated viral (AAV) vectors, simian viral vectors, vaccinia viral vectors, Sendai viral vectors, Epstein-Barr viral (EBV) vectors, and HSV vectors. Specific examples of the linking vectors include pUC, pCMV, pMX, and pELP. The linking vectors can also be determined as appropriate in accordance with hosts into which the expression vectors are to be introduced, for example. There is no particular limitation on the hosts, and examples thereof include mammalian-derived cultured cells such as CHO cells, Jurkat cells, and Jurkat 76 cells, and immune cells. Examples of the immune cells include lymphocytes, granulocytes, and macrophages. Examples of the lymphocytes include T cells, NK cells, NKT cells, and B cells. The immune cells are cells isolated from a living organism, immune cells induced from stem cells such as multipotent stem cells, or cultured cells derived from immune cells, for example. The T cells may be T cell-like cells. Examples of the T cell-like cells include cultured cells derived from T cells, and specific examples thereof include Jurkat cells and Jurkat 76 cells.

It is preferable that each of the expression vectors includes a regulatory sequence for regulating at least either the expression of the nucleic acid coding for the first BsAb or the expression of the first BsAb encoded by the nucleic acid coding for the first BsAb, for example. Examples of the regulatory sequence include a promoter, a terminator, an enhancer, a polyadenylation signal sequence, and a replication origin sequence (ori). There is no particular limitation on the arrangement of the regulatory sequence in the expression vector. It is sufficient that the regulatory sequence is arranged in the expression vector such that at least either the expression of the nucleic acid coding for the first BsAb or the expression of the first BsAb encoded by the nucleic acid can be functionally regulated, for example, and the regulatory sequence can be arranged based on a known method. For example, a predetermined sequence included in the linking vector may be used as the regulatory sequence, or an additional regulatory sequence may be inserted into the linking vector, or a regulatory sequence included in the linking vector may be replaced with another regulatory sequence.

The expression vector may further include a sequence coding for a selective marker, for example. Examples of the selective marker include drug-resistant markers, fluorescent protein markers, enzymatic markers, and cell-surface receptor markers.

### Second Screening Method

A second scFv-screening method (also referred to as a “second screening method” hereinafter) of the present invention includes:
a first production step of producing first BsAbs from the BsAb library of the present invention;
a first contact step in which, when the first target antigen is an immune cell-activating receptor,
the first BsAbs, the second target antigen, and immune cells capable of expressing the immune cell-activating receptor are brought into contact with one another, and
when the second target antigen is an immune cell-activating receptor,
the first BsAbs, the first target antigen, and immune cells capable of expressing the immune cell-activating receptor are brought into contact with one another; and
a first selection step of selecting second scFvs of the first BsAbs that have bound to the second target antigen in the first contact step as first candidate scFvs capable of binding to the second target antigen.

The second screening method of the present invention is characterized by including the first production step, the first contact step, and the first selection step, and using the BsAb library of the present invention in the first production step, and there is no particular limitation on the other configurations and conditions. With the second screening method of the present invention, scFvs capable of binding to the second target antigen can be screened more easily compared with a method in which hybridomas are used and a method in which phage display is used, for example. Moreover, with the second screening method of the present invention, BsAbs that can induce cytotoxic activity and the like against cells expressing the first target antigen or second target antigen via T cells can be screened, for example. Furthermore, an antibody capable of binding to the target antigen or an antigen-binding fragment thereof can be produced based on the amino acid sequences of the CDRs in the heavy-chain variable regions and the light-chain variable regions of scFvs capable of binding to the second target antigen, for example. Accordingly, the second screening method can also be considered as a method for screening an antibody capable of binding to the target antigen or an antigen-binding fragment of the antibody, for example. The descriptions of the CAR library, the first screening method, the first antibody or the like, the second antibody or the like, the gene, the expression vector, the transformant, the first chimeric antigen receptor, the second chimeric antigen receptor, the nucleic acid, the cell, the cell-manufacturing method, the therapeutic agent, the treatment method, the BsAb library, and the like of the present invention can be applied to the second screening method of the present invention, for example. The second screening method of the present invention can be used to manufacture new scFvs capable of binding to a target antigen, for example. Accordingly, the second screening method of the present invention can also be considered as an scFv-manufacturing method, for example.

When BsAbs and the like capable of binding to a target antigen are manufactured using a phage display technique or the like, an antibody library in which the number of types of antibodies is about 1×10¹⁰ is produced, and antibodies capable of binding to the target antigen are obtained, followed by preparation of BsAbs and the like that include scFvs from the antibodies. Then, the effectiveness of the BsAbs and the like is examined using immune cells or the like. On the other hand, in the second screening method of the present invention, the BsAb library of the present invention and the immune cells are used, thus making it possible to simultaneously perform screening of scFvs capable of binding to the target antigen and examination of the effectiveness of the BsAbs expressed from the library in the immune cells, for example. Accordingly, with the second screening method of the present invention, scFvs capable of binding to a target antigen and being used for BsAbs that are functional in immune cells and the like can be more easily screened compared with a phage display technique and the like used to obtain antibodies capable of binding to a target antigen, for example.

The first production step is a step of producing first BsAbs from the BsAb library of the present invention. There is no particular limitation on the method for producing first BsAbs, and a known method in which a protein is synthesized from a nucleic acid can be used, for example. In a specific example, in the first production step, the BsAb library (first BsAb library) of the present invention is introduced into hosts, for example. Then, in the first production step, the BsAbs are expressed in the hosts by culturing the hosts into which the first BsAb library has been introduced, for example. The method for introducing the first BsAb library is not particularly limited, and can be determined as appropriate in accordance with the type of hosts, for example. When the hosts are cells, a known method for introducing a nucleic acid into cells can be used as the above-mentioned introducing method, for example, and the description of the introducing method in the above-mentioned first expression step of the first screening method of the present invention can be applied. The host culture conditions are not particularly limited, and can be determined as appropriate in accordance with the type of hosts, for example. In a specific example, when the hosts are cells, the cells are cultured for 24 hours to 2 weeks, 24 to 48 hours, or 1 to 2 weeks, for example, but there is no particular limitation thereto. The cells are cultured at a temperature of 28 to 37° C., for example.

The first BsAb library may include nucleic acids coding for the first BsAbs, or expression vectors into which nucleic acids coding for the first BsAbs have been introduced, for example. The descriptions of the BsAb library of the present invention can be applied to the above-mentioned hosts, for example. The hosts are preferably 293 cells. The first BsAbs are preferably tandem scFvs because the library can be easily constructed.

Next, in the first contact step, the first BsAbs, the target antigen, and the immune cells capable of expressing the immune cell-activating receptor are brought into contact with one another. Specifically, when the first target antigen of the first BsAbs is an immune cell-activating receptor, the first BsAbs, the second target antigen, and the immune cells are brought into contact with one another in the first contact step. On the other hand, when the second target antigen of the first BsAbs is an immune cell-activating receptor, the first BsAbs, the first target antigen, and the immune cells are brought into contact with one another in the first contact step. The first BsAbs, the target antigen, and the immune cells can be brought into contact with one another by culturing the first BsAbs, the target antigen, and the immune cells together, for example. These are cultured for 1 to 96 hours, 1 to 6 hours, or 6 to 96 hours, for example. These are cultured at a temperature of 28 to 37° C., for example. It is preferable that the ratio between the number of the immune cells and the number of molecules of the first BsAbs is determined such that the number of molecules of the first BsAbs is 0.1 pmol or more for 1×10⁶ immune cells, for example.

The immune cells capable of expressing the immune cell-activating receptor may by immune cells expressing the immune cell-activating receptor, or immune cells capable of expressing the immune cell-activating receptor under predetermined conditions. The predetermined conditions can be determined as appropriate in accordance with the type of immune cells, for example. The immune cells can be selected as appropriate in accordance with the type of immune cell-activating receptor. When the immune cell-activating receptor is CD3, examples of the immune cells include T cells and NKT cells. When the immune cell-activating receptor is an NK cell-activating receptor, examples of the immune cells include NK cells and NKT cells. When the immune cell-activating receptor is CD19, CD79a, or CD79B, examples of the immune cells include B cells. The above-mentioned T cells, NK cells, NKT cells, and B cells may be the above-described T cell-like cultured cells, NK cell-like cultured cells, NKT cell-like cultured cells, and B cell-like cultured cells, respectively. It is preferable that the immune cell-activating receptor and immune cells are CD3, and T cells or T cell-like cultured cells, respectively. Accordingly, with the second screening method of the present invention, BsAbs that are likely to induce cytotoxic activity against cells expressing the first target antigen or second target antigen via T cells can be screened, for example.

The number of the types of first BsAbs may be one or two or more, for example. In the latter case, a mixture of a plurality of types of first BsAbs having different amino acid sequences is used as the first BsAbs in the first contact step, for example.

When the first target antigen is an immune cell-activating receptor, examples of the second target antigen to be used in the contact in the first contact step include a second target antigen monomer, a second target antigen complex, and a second target antigen-expressing cell, and a second target antigen-expressing cell is preferable. An example of the second target antigen complex is a second target antigen multimer, and specific examples thereof include a target antigen dimer and a target antigen tetramer. The second target antigen multimer can be prepared using a method in which tagged target antigens are cross-linked via an antibody, a method in which a complex of biotinylated target antigens is formed using avidin, or the like, for example. Examples of the second target antigen-expressing cell include cells that intrinsically express the second target antigen, and cells that express the second target antigen due to the introduction of a nucleic acid coding for the second target antigen. Examples of the second target antigen-expressing cell include 293 cells, 293 T cells, and K562 cells. The cell in which the second target antigen is to be expressed may be the same as or different from the above-mentioned host, for example.

When the second target antigen is an immune cell-activating receptor, examples of the first target antigen to be used in the contact in the first contact step include a first target antigen monomer, a first target antigen complex, and a first target antigen-expressing cell, and a first target antigen-expressing cell is preferable. An example of the first target antigen complex is a first target antigen multimer, and specific examples thereof include a target antigen dimer and a target antigen tetramer. The first target antigen multimer can be prepared using a method in which tagged target antigens are cross-linked via an antibody, a method in which a complex of biotinylated target antigens is formed using avidin, or the like, for example. Examples of the first target antigen-expressing cell include cells that intrinsically express the first target antigen, and cells that express the first target antigen due to the introduction of a nucleic acid coding for the first target antigen. Examples of the first target antigen-expressing cell include cultured cells such as 293 cells, 293 T cells, and K562 cells. The cell in which the first target antigen is to be expressed may be the same as or different from the above-mentioned hosts, for example.

Then, in the first selection step, the second scFvs of the first BsAbs that have bound to the second target antigen in the first contact step are selected as first candidate scFvs capable of binding to the second target antigen. The binding of the first BsAbs to the second target antigen can be evaluated directly or indirectly, for example, and indirect evaluation is preferable because BsAbs that can induce cytotoxic activity and the like against cells expressing the first target antigen or second target antigen via T cells can be screened, for example.

The direct evaluation method can be performed using a technique for detecting the binding of an antibody to an antigen such as surface plasmon resonance (SPR) or flow cytometry, for example. In a specific example, the direct evaluation method can be performed using a labeled first target antigen monomer or multimer, or a labeled second target antigen monomer or multimer, for example. In this case, in the first selection step, the first BsAbs, the labeled first target antigen or second target antigen, and the immune cells are brought into contact with one another, for example. When the first BsAbs bind to the second target antigen, the first BsAb, the labeled first target antigen or second target antigen, and the immune cell form a complex. Accordingly, in the first selection step, it can be determined that the first BsAb have bound to the second target antigen if some of the immune cells form a complex that includes the above-mentioned label, for example.

The indirect evaluation method can be implemented as follows, for example. The first BsAbs capable of binding to the second target antigen form cross-links between the immune cell-activating receptors of the immune cells, for example. As a result, the immune cells that include the cross-linked immune cell-activating receptors are activated by the signal generated in the immune cell-activating receptors, for example. Accordingly, in the indirect evaluation method, the activation of the immune cells is used as an evaluation index, for example. In a specific example, when the first BsAbs bind to the second target antigen, the expression of activation markers is increased in the immune cells, the production amounts of cytokines and/or chemokines are increased in the immune cells, and the immune cells are proliferated, for example, compared with the case where the first BsAbs that do not bind to the second target antigen are present, for example. In a specific example, when the immune cells are T cells, NK cells, NKT cells, or B cells, the expression of the following activation markers is increased in the cells, the production amounts of the following cytokines and/or chemokines are increased in the cells, and the cells are proliferated, for example. Accordingly, in the first selection step, when it is determined based on any one or more of the indices that the immune cells are activated, it can be determined that the first BsAbs that have activated the immune cells have bound to the second target antigen. The increase in the production amounts of cytokines and/or chemokines may be evaluated using a reporter whose mRNA or protein expression level increases when the expression of the cytokines and/or chemokines is induced, for example. An example of the reporter is a fluorescent protein.

### T Cells

Activation marker: CD69, CD107a, etc.
Cytokine, chemokine: IFN-γ, IL-12, IL-2, TNFα, MIP-1β, etc.

### NK Cells

Activation marker: CD69, CD107a, etc.
Cytokine, chemokine: INF-γ, IL-12, etc.

### NKT Cells

Activation marker: CD69, CD107a, CD25, etc.
Cytokine, chemokine: INF-γ, IL-2, etc.
   b cells
Activation marker: CD28, CD69, CD80, CD138, B220, etc.
Cytokine, chemokine: CXCR4, etc.

Then, the second scFvs of the first BsAbs that have bound to the second target antigen are selected as first candidate scFvs capable of binding to the second target antigen. The first candidate scFvs can be selected by selecting hosts expressing the first BsAbs capable of binding to the second target antigen and reading the base sequences coding for the scFvs or BsAbs in the selected hosts, for example. In the first selection step, the CDRH1, the CDRH2, and the CDRH3 in the heavy-chain variable region and the CDRL1, the CDRL2, and the CDRL3 in the light-chain variable region in each of the first candidate scFvs may also be identified, for example. The CDRs can be identified using a known method referring to the genome information, for example.

Accordingly, new scFvs capable of binding to the second target antigen can be screened. If a first BsAb of the first BsAb library meets Condition 1 above, the first screening method of the present invention can be used to screen new light-chain variable regions capable of binding to the second target antigen. If a first BsAb of the first BsAb library meets Condition 2 above, the first screening method of the present invention can be used to screen new heavy-chain variable regions capable of binding to the second target antigen.

When the first BsAbs include a plurality of types of BsAbs, the first contact step and the first selection step may be performed once or a plurality of times, and are preferably performed a plurality of times. The term “plurality of times” refers to two to five times or two to four times, for example, and preferably two to three times. In this case, it is preferable that the first contact step and the first selection step are performed as one set, and this set is performed a plurality of times. When the first contact step and the first selection step are performed a plurality of times, the first round of the first contact step and the first selection step is performed, and then new first BsAbs are produced from the hosts expressing first BsAbs capable of binding to the second target antigen, for example. In this case, it is preferable that the number of types of BsAbs included in the new first BsAbs is smaller than the number of types of BsAbs subjected to the previous round of the first contact step and the first selection step. The new first BsAbs are diluted after the hosts expressing the first BsAbs have been collected, for example. The new first BsAbs can be prepared by reseeding the diluted hosts at a cell density lower than that at the time of collection, culturing the hosts, and collecting the culture supernatant. In the second screening method of the present invention, the first contact step and the first selection step are performed using the new first BsAbs, for example. The contact step and the selection step are repeatedly performed a desired number of times in the same manner, and thus a monoclonal host expressing a first BsAb capable of binding to the second target antigen is obtained, for example. Accordingly, with the second screening method of the present invention, first BsAbs capable of binding to the second target antigen can be identified more reliably, for example.

In the second screening method of the present invention, the new heavy-chain variable region or light-chain variable region of the first candidate scFv may be used as the heavy-chain variable region or light-chain variable region of an antibody or the like capable of binding to the second target antigen out of the second scFvs of the first BsAb library to screen the other region, for example. In this case, the second screening method of the present invention further includes a preparation step of preparing a second BsAb library based on the first candidate scFvs, for example.

The second BsAb library includes nucleic acids coding for second BsAbs, for example. Each of the second BsAbs includes a third antigen-binding domain and a fourth antigen-binding domain, for example. The third antigen-binding domain includes a third scFv capable of binding to the first target antigen, for example. The fourth antigen-binding domain includes a fourth scFv to be screened for the ability to bind to the second target antigen, for example.

The nucleic acids coding for the second BsAbs are nucleic acids (polynucleotides) coding for the amino acid sequences of the second BsAbs, for example.

In the second screening method of the present invention, the second BsAbs and the first BsAbs may have the same structure or different structures, for example. The second BsAbs are preferably tandem scFvs because the library can be easily constructed.

When the first target antigen is an immune cell-activating receptor, the first target antigen of the second BsAbs may be the same as or different from the first target antigen of the first BsAbs of the above-mentioned BsAb library of the present invention described in “(1) First Target Antigen: Immune Cell-Activating Receptor”. In the latter case, the first target antigen of the second BsAbs is a subunit or complex different from the first target antigen of the first BsAbs, for example.

When the second target antigen is an immune cell-activating receptor, the first target antigen of the second BsAbs is the same as the first target antigen of the first BsAbs of the above-mentioned BsAb library of the present invention described in “(2) Second Target Antigen: Immune Cell-Activating Receptor”, and the descriptions thereof can be applied.

When the first target antigen is an immune cell-activating receptor, the second target antigen of the second BsAbs is the same as the second target antigen of the first BsAbs of the above-mentioned BsAb library of the present invention described in “(1) First Target Antigen: Immune Cell-Activating Receptor”, and the descriptions thereof can be applied.

When the second target antigen is an immune cell-activating receptor, the second target antigen of the second BsAbs may be the same as or different from the second target antigen of the first BsAbs of the above-mentioned BsAb library of the present invention described in “(2) Second Target Antigen: Immune Cell-Activating Receptor”. In the latter case, the second target antigen of the second BsAbs is a subunit or complex different from the first target antigen of the first BsAbs, for example.

If the “first scFv” is changed to the “third scFv”, the “first heavy-chain variable region” is changed to a “third heavy-chain variable region”, and the “first light-chain variable region” is changed to a “third light-chain variable region”, the descriptions of the first scFv of the BsAb library of the present invention described in “(1) First Target Antigen: Immune Cell-Activating Receptor” can be applied to the third scFv, for example. The third scFv of the second BsAb may be the same as or different from the first scFv of the first BsAb.

In each of the third scFvs, the third heavy-chain variable region and the third light-chain variable region are coupled to each other via a third linker peptide (third Fv linker peptide), for example. It is preferable that the third Fv linker peptide does not inhibit the third scFv from binding to the second target antigen, for example. The third Fv linker peptide is constituted by 1 to 40, 1 to 18, 1 to 15, 1 to 7, 1 to 3, or 1 or 2 amino acids, for example. The third Fv linker peptide is constituted by amino acids such as glycine and serine, for example, and a specific example thereof is (GGGGS)ₙ. n in this formula is an integer from 1 to 6, for example. The amino acid sequence of the third Fv linker peptide may be the amino acid sequence of the polypeptide represented by Sequence ID No. 1 or 2 above, for example.

In each of the second BsAbs, the fourth scFv includes a fourth heavy-chain variable region and a fourth light-chain variable region. The fourth heavy-chain variable region and the fourth light-chain variable region have structures similar to those of the heavy-chain variable region and the light-chain variable region in an antibody molecule, respectively, and the descriptions of the first scFv can be applied to the structures.

The fourth heavy-chain variable region includes a CDRH1, a CDRH2, and a CDRH3. The fourth light-chain variable region includes a CDRL1, a CDRL2, and a CDRL3. The fourth heavy-chain variable region and the fourth light-chain variable region meet Condition 3 or Condition 4 below.

### Condition 3:

If the BsAb library in the first production step meets Condition 1 above,
the CDRH1, the CDRH2, and the CDRH3 in the fourth heavy-chain variable region include the CDRH1, the CDRH2, and the CDRH3 in the heavy-chain variable region of a second B cell receptor, respectively, and
the CDRL1, the CDRL2, and the CDRL3 in the fourth light-chain variable region include the CDRL1, the CDRL2, and the CDRL3 in the light-chain variable region of the first candidate BsAb, respectively.

### Condition 4:

If the BsAb library in the first production step meets Condition 2 above,
the CDRH1, the CDRH2, and the CDRH3 in the fourth heavy-chain variable region include the CDRH1, the CDRH2, and the CDRH3 in the heavy-chain variable region of the first candidate BsAb, respectively, and
the CDRL1, the CDRL2, and the CDRL3 in the fourth light-chain variable region include the CDRL1, the CDRL2, and the CDRL3 in the light-chain variable region of a second B cell receptor, respectively.

Condition 3 above is employed in the case where the second BsAb library is prepared using the first candidate scFvs screened using the first BsAb library that meets Condition 1 above, for example. In Condition 3 above, the first candidate scFv is used for the CDRL1, the CDRL2, and the CDRL3 in the fourth light-chain variable region, and the CDRH1, the CDRH2, and the CDRH3 in the fourth heavy-chain variable region are screened for the ability to bind to the second target antigen, for example.

If the “first B cell receptor” is changed to the “second B cell receptor”, and the “second heavy-chain variable region” is changed to the “fourth heavy-chain variable region”, the descriptions of the second heavy-chain variable region of the above-mentioned BsAb library of the present invention described in Condition 2 in “(1) First Target Antigen: Immune Cell-Activating Receptor” can be applied to the second B cell receptor in the fourth heavy-chain variable region, for example.

The CDRH1, the CDRH2, and the CDRH3 in the fourth heavy-chain variable region may respectively be polypeptides that consist of the amino acid sequences of the CDRH1, the CDRH2, and the CDRH3 of the second B cell receptor, or polypeptides that include the amino acid sequences of the CDRH1, the CDRH2, and the CDRH3 of the second B cell receptor, for example.

The FRH1, the FRH2, the FRH3, and the FRH4 in the fourth heavy-chain variable region may respectively be polypeptides that consist of the amino acid sequences of the FRH1, the FRH2, the FRH3, and the FRH4 of the second B cell receptor, or polypeptides that include the amino acid sequences of the FRH1, the FRH2, the FRH3, and the FRH4 of the second B cell receptor, for example. It is preferable that “the second B cell receptor” in the descriptions of the CDRHs and “the second B cell receptor” in the descriptions of the FRHs are the same B cell receptor.

The fourth heavy-chain variable region may include the heavy-chain variable region of the second B cell receptor, for example. In this case, the fourth heavy-chain variable region may be a polypeptide that consists of the amino acid sequence of the heavy-chain variable region of the second B cell receptor, or a polypeptide that includes the amino acid sequence of the heavy-chain variable region of the second B cell receptor, for example.

The CDRL1, the CDRL2, and the CDRL3 in the fourth light-chain variable region may respectively be polypeptides that consist of the amino acid sequences of the CDRL1, the CDRL2, and the CDRL3 of the first candidate scFv, or polypeptides that include the amino acid sequences of the CDRL1, the CDRL2, and the CDRL3 of the first candidate scFv, for example.

The FRs in the fourth light-chain variable region may include the FRs in the light-chain variable region of the first candidate scFv, for example. The FRL1, the FRL2, the FRL3, and the FRL4 in the fourth light-chain variable region may respectively be polypeptides that consist of the amino acid sequences of the FRL1, the FRL2, the FRL3, and the FRL4 of the first candidate scFv, or polypeptides that include the amino acid sequences of the FRL1, the FRL2, the FRL3, and the FRL4 of the first candidate scFv, for example. It is preferable that “the first candidate scFv” in the descriptions of the CDRLs and “the first candidate scFv” in the descriptions of the FRLs are the same scFv.

The fourth light-chain variable region may include the light-chain variable region of the first candidate scFv, for example. In this case, the fourth light-chain variable region may be a polypeptide that consists of the amino acid sequence of the light-chain variable region of the first candidate scFv, or a polypeptide that includes the amino acid sequence of the light-chain variable region of the first candidate scFv, for example.

Next, Condition 4 above is employed in the case where the second BsAb library is prepared using the first candidate scFvs screened using the first BsAb library that meets Condition 2 above, for example. In Condition 4 above, the first candidate scFv is used for the CDRH1, the CDRH2, and the CDRH3 in the fourth heavy-chain variable region, and the CDRL1, the CDRL2, and the CDRL3 in the fourth light-chain variable region are screened for the ability to bind to the second target antigen, for example.

The CDRH1, the CDRH2, and the CDRH3 in the fourth heavy-chain variable region may respectively be polypeptides that consist of the amino acid sequences of the CDRH1, the CDRH2, and the CDRH3 of the first candidate scFv, or polypeptides that include the amino acid sequences of the CDRH1, the CDRH2, and the CDRH3 of the first candidate scFv, for example.

The FRH1, the FRH2, the FRH3, and the FRH4 in the fourth heavy-chain variable region may respectively be polypeptides that consist of the amino acid sequences of the FRH1, the FRH2, the FRH3, and the FRH4 of the first candidate scFv, or polypeptides that include the amino acid sequences of the FRH1, the FRH2, the FRH3, and the FRH4 of the first candidate scFv, for example. It is preferable that “the first candidate scFv” in the descriptions of the CDRHs and “the first candidate scFv” in the descriptions of the FRHs are the same scFv.

The fourth heavy-chain variable region may include the heavy-chain variable region of the first candidate scFv, for example. In this case, the fourth heavy-chain variable region may be a polypeptide that consists of the amino acid sequence of the heavy-chain variable region of the first candidate scFv, or a polypeptide that includes the amino acid sequence of the heavy-chain variable region of the first candidate scFv, for example.

If the “first B cell receptor” is changed to the “second B cell receptor”, and the “second light-chain variable region” is changed to the “fourth light-chain variable region”, the descriptions of the second light-chain variable region of the above-mentioned BsAb library of the present invention described in Condition 1 in “(1) First Target Antigen: Immune Cell-Activating Receptor” can be applied to the second B cell receptor in the fourth light-chain variable region, for example.

The CDRL1, the CDRL2, and the CDRL3 in the fourth light-chain variable region may respectively be polypeptides that consist of the amino acid sequences of the CDRL1, the CDRL2, and the CDRL3 of the second B cell receptor, or polypeptides that include the amino acid sequences of the CDRL1, the CDRL2, and the CDRL3 of the second B cell receptor, for example.

The FRL1, the FRL2, the FRL3, and the FRL4 in the fourth light-chain variable region may respectively be polypeptides that consist of the amino acid sequences of the FRL1, the FRL2, the FRL3, and the FRL4 of the second B cell receptor, or polypeptides that include the amino acid sequences of the FRL1, the FRL2, the FRL3, and the FRL4 of the second B cell receptor, for example. It is preferable that “the second B cell receptor” in the descriptions of the CDRLs and “the second B cell receptor” in the descriptions of the FRLs are the same B cell receptor.

The fourth light-chain variable region may include the light-chain variable region of the second B cell receptor, for example. In this case, the fourth light-chain variable region may be a polypeptide that consists of the amino acid sequence of the light-chain variable region of the second B cell receptor, or a polypeptide that includes the amino acid sequence of the light-chain variable region of the second B cell receptor, for example.

In each of the fourth scFvs, the fourth heavy-chain variable region and the fourth light-chain variable region are coupled to each other via a fourth linker peptide (fourth Fv linker peptide), for example. It is preferable that the fourth Fv linker peptide does not inhibit the fourth scFv from binding to the immune cell-activating receptor, for example. The fourth Fv linker peptide is constituted by 1 to 40, 1 to 18, 1 to 15, 1 to 7, 1 to 3, or 1 or 2 amino acids, for example. The fourth Fv linker peptide is constituted by amino acids such as glycine and serine, for example, and a specific example thereof is (GGGGS)ₙ. n in this formula is an integer from 1 to 6, for example. The amino acid sequence of the fourth Fv linker peptide may be the amino acid sequence of the polypeptide represented by Sequence ID No. 1 or 2 above, for example. The fourth Fv linker peptide in the fourth scFv may be the same as or different from the first Fv linker peptide in the first scFv, for example.

The third scFv and the fourth scFv may be directly or indirectly coupled to each other, or be formed such that, when the third scFv and the fourth scFv coexist, the third scFv and the fourth scFv are associated with (bind to) each other, for example. The coupling between the third scFv and the fourth scFv can be designed as appropriate in accordance with the type of second BsAb, for example. In a specific example, when the second BsAb is a tandem scFv, the third scFv and the fourth scFv are coupled to each other via a linker peptide (inter-Fv linker peptide). The inter-Fv linker peptide is constituted by 1 to 40, 1 to 18, 1 to 15, 1 to 7, 1 to 3, or 1 or 2 amino acids, for example. The inter-Fv linker peptide is constituted by amino acids such as glycine and serine, for example, and a specific example thereof is (GGGGS)ₙ. n in this formula is an integer from 1 to 6, for example. In a specific example, the amino acid sequence of the inter-Fv linker peptide may be the amino acid sequence of the polypeptide represented by Sequence ID No. 1 or 2 above, for example. The electric charges of the amino acid side chains can be used for the above-mentioned association, for example. In a specific example, the association can be induced by adding a domain that includes positively charged amino acids to one binding domain and adding a domain that includes negatively charged amino acids to the other binding domain, for example. Moreover, a combination of a tag sequence and a polypeptide capable of recognizing the tag sequence, or the like can also be used for the association, for example.

Regarding the arrangement order of the third scFv and the fourth scFv of each of the second BsAbs, the third scFv and the fourth scFv may be arranged in this order from the N-terminal side, or the fourth scFv and the third scFv may be arranged in this order from the N-terminal side.

An example of the second BsAb is a polypeptide consisting of the amino acid sequence represented by Formula (5) (Sequence ID No. 160) below. Examples of the combination of V₅, V₆, V₇, and V₈ in Formula (5) below include combinations (V9) to (V16) below. When the fourth scFv meets Condition 3 above, the combination of V₅, V₆, V₇, and V₈ is preferably the combination (V13) or (V14) below. When the fourth scFv meets Condition 4 above, the combination of V₅, V₆, V₇, and V₈ is preferably the combination (V15) or (V16) below. In Formula (5) below, the amino acid sequence between V₅ and V₆ is the amino acid sequence of the first Fv linker peptide, the amino acid sequence between V₆ and V₇ is the amino acid sequence of the inter-Fv linker peptide, and the amino acid sequence between V₇ and V₈ is the amino acid sequence of the second Fv linker peptide. In Formula (5) below, the first Fv linker peptide and the second Fv linker peptide are Fv linker peptides 1, but may also be independently an Fv linker peptide 2.

[V₅]-[GSTSGSGKPGSGEGSTKG]-[V₆]-[SGSG]-[V₇]-
[GSTSGSGKPGSGEGSTKG]-[V₈]......(5)

(V9) V₅: V_{H3}, V₆: V_{L3}, V₇: V_{H4}, V₈: V_{L4}
(V10) V₅: V_{L3}, V₆: V_{H3}, V₇: V_{H4}, V₈: V_{L4}
(V11) V₅: V_{H3}, V₆: V_{L3}, V₇: V_{L4}, V₈: V_{H4}
(V12) V₅: V_{L3}, V₆: V_{H3}, V₇: V_{L4}, V₈: V_{H4}
(V13) V₅: V_{H4}, V₆: V_{L4}, V₇: V_{H3}, V₈: V_{L3}
(V14) V₅: V_{H4}, V₆: V_{L4}, V₇: V_{L3}, V₈: V_{H3}
(V15) V₅: V_{L4}, V₆: V_{H4}, V₇: V_{H3}, V₈: V_{L3}
(V16) V₅: V_{L4}, V₆: V_{H4}, V₇: V_{L3}, V₈: V_{H3}
   V_{H3}: third heavy-chain variable region
   V_{L3}: third light-chain variable region
   V_{H4}: fourth heavy-chain variable region
   V_{L4}: fourth light-chain variable region

An example of the nucleic acid coding for the second BsAb is a polynucleotide consisting of the base sequence represented by Formula (6) (Sequence ID No. 161) below. Examples of the combination of N₅, N₆, N₇, and N₈ in Formula (6) below include combinations (N9) to (N16) below. The combinations (N9) to (N16) below are nucleic acids coding for the second BsAbs of the combinations (V9) to (V16) above, respectively. In Formula (6) below, the base sequence between N₅ and N₆ is the base sequence coding for the first Fv linker peptide, the base sequence between N₆ and N₇ is the base sequence coding for the inter-Fv linker peptide, and the base sequence between N₇ and N₈ is the base sequence coding for the second Fv linker peptide. In Formula (6) below, the base sequences coding for the first Fv linker peptide and the second Fv linker peptide are base sequences coding for Fv linker peptides 1, but may also be independently a base sequence coding for an Fv linker peptide 2.

5′-[N₅]-[GGCTCTACAAGCGGCTCTGGCAAGCCTGGATCTGGCGAG
GGAAGCACCAAGGGC]-[N₆]-[AGCGGATCTGGC]-[N₇]-[GGCTC
TACAAGCGGCTCTGGCAAGCCTGGATCTGGCGAGGGAAGCACCAAGG
GC]-[N₈]-3′......(6)

(N9) N₅: N_{H3}, N₆: N_{L3}, N₇: N_{H4}, N₈: N_{L4}
(N10) N₅: N_{L3}, N₆: N_{H3}, N₇: N_{H4}, N₈: N_{L4}
(N11) N₅: N_{H3}, N₆: N_{L3}, N₇: N_{L4}, N₈: N_{H4}
(N12) N₅: N_{L3}, N₆: N_{H3}, N₇: N_{L4}, N₈: N_{H4}
(N13) N₅: N_{H4}, N₆: N_{L4}, N₇: N_{H3}, N₈: N_{L3}
(N14) N₅: N_{H4}, N₆: N_{L4}, N₇: N_{L3}, N₈: N_{H3}
(N15) N₅: N_{L4}, N₆: N_{H4}, N₇: N_{H3}, N₈: N_{L3}
(N16) N₅: N_{L4}, N₆: N_{H4}, N₇: N_{L3}, N₈: N_{H3}
   N_{H3}: base sequence coding for third heavy-chain variable region
   N_{L3}: base sequence coding for third light-chain variable region
   N_{H4}: base sequence coding for fourth heavy-chain variable region
   N_{L4}: base sequence coding for fourth light-chain variable region

It is preferable that the second BsAb library includes a plurality of types of nucleic acids, for example. In this case, the second BsAb library is a mixture of a plurality of types of nucleic acids, for example. It is preferable that some or all of the plurality of types of nucleic acids code for different second BsAbs, and preferably code for different fourth antigen-binding domains, for example. When the plurality of types of nucleic acids code for different fourth antigen-binding domains, the regions other than the fourth antigen-binding domains in the second BsAbs have the same amino acid sequence or different amino acid sequences, for example. The number of types of nucleic acids included in the second BsAb library is 1×10⁵ to 1×10⁶ or 1×10⁶ to 5×10⁶, for example, and preferably about 1×10⁶ (e.g., 8×10⁵ to 2×10⁶).

The second BsAb may include a signal peptide at the N terminus, for example. The second BsAb may include a tag, for example. If the “first BsAb” is changed to the “second BsAb”, the descriptions of the signal peptide and the tag in the first BsAb can be applied to the above-mentioned signal peptide and the tag, for example.

In the present invention, the nucleic acids coding for the second BsAbs can be prepared based on the amino acid sequences of the second BsAbs using an ordinary method, for example. In a specific example, the nucleic acids coding for the second BsAbs can be prepared based on the base sequences coding for the amino acid sequences obtained from the database in which the amino acid sequences of the above-described domains are registered, using a molecular biological technique and/or a chemical synthesis method, for example. The base sequences of the nucleic acids may be subjected to codon optimization in accordance with the source of cells in which the second BsAb library is to be expressed, for example.

The nucleic acids coding for the second BsAbs may be introduced into expression vectors, for example. The descriptions of the expression vectors for the BsAb library of the present invention can be applied to the above-mentioned expression vectors, for example.

Next, in the second screening method of the present invention, a production step, a contact step, and a selection step are performed in the same manner, except that the second BsAb library prepared in the above-mentioned preparation step is used instead of the first BsAb library, for example. Specifically, the second screening method of the present invention further includes a second production step of producing the second BsAbs from the second BsAb library, a second contact step of bringing the second BsAbs, the target antigen, and immune cells capable of expressing the immune cell-activating receptor into contact with one another, and a second selection step of selecting fourth scFvs of the second BsAbs that have bound to the second target antigen in the second contact step as second candidate scFvs capable of binding to the second target antigen, for example. When the first target antigen of the second BsAbs is an immune cell-activating receptor, the second BsAbs, the second target antigen, and the immune cells are brought into contact with one another in the second contact step, for example. On the other hand, when the second target antigen of the second BsAbs is an immune cell-activating receptor, the second BsAbs, the first target antigen, and the immune cells are brought into contact with one another in the second contact step, for example.

If the “first production step” is changed to the “second production step”, the “first BsAb library” is changed to the “second BsAb library”, and the “first BsAb” is changed to the “second BsAb”, the descriptions of the first production step can be applied to the second production step, for example.

If the “first production step” is changed to the “second production step”, and the “first contact step” is changed to the “second contact step”, the descriptions of the first contact step can be applied to the second contact step, for example.

If the “first contact step” is changed to the “second contact step”, the “first selection step” is changed to the “second selection step”, the “second scFv” is changed to the “fourth scFv”, the “first candidate scFv” is changed to the “second candidate scFv”, and the “first BsAb” is changed to the “second BsAb”, the descriptions of the first selection step can be applied to the second selection step, for example.

Accordingly, the second screening method of the present invention can be used to screen scFvs that include new heavy-chain variable regions and light-chain variable regions capable of binding to the second target antigen, for example.

Method for Manufacturing Third scFv

As described above, the scFv-manufacturing method of the present invention includes: a first administration step of administering cells expressing a first chimeric antigen receptor (CAR) library to an animal, and
a first collection step of collecting the cells expressing the first CAR library that accumulate in a tissue expressing a target antigen in the animal as cells expressing CARs specific to the target antigen,
wherein the first CAR library includes nucleic acids coding for first CARs,
each of the first CARs includes a first antigen-binding domain, a first transmembrane domain, and a first intracellular signaling domain,
the first antigen-binding domain includes a first single-chain antibody (scFv) to be screened for the ability to bind to the target antigen,
the first scFv includes a first heavy-chain variable region and a first light-chain variable region, and
the first heavy-chain variable region and the first light-chain variable region meet Condition 1 or Condition 2 below.

### Condition 1

The heavy-chain complementarity determining region (CDRH) 1, the CDRH2, and the CDRH3 in the first heavy-chain variable region include the CDRH1, the CDRH2, and the CDRH3 in the heavy-chain variable region of an antibody capable of binding to the target antigen or an antigen-binding fragment of the antibody, respectively, and
the light-chain complementarity determining region (CDRL) 1, the CDRL2, and the CDRL3 in the first light-chain variable region include the CDRL1, the CDRL2, and the CDRL3 in the light-chain variable region of a first B cell receptor, respectively.

### Condition 2

The heavy-chain complementarity determining region (CDRH) 1, the CDRH2, and the CDRH3 in the first heavy-chain variable region include the CDRH1, the CDRH2, and the CDRH3 in the heavy-chain variable region of a first B cell receptor, respectively, and
the light-chain complementarity determining region (CDRL) 1, the CDRL2, and the CDRL3 in the first light-chain variable region include the CDRL1, the CDRL2, and the CDRL3 in the light-chain variable region of an antibody capable of binding to the target antigen or an antigen-binding fragment of the antibody, respectively.

A third scFv-manufacturing method of the present invention is characterized by including the first administration step of administering cells expressing a first CAR library to an animal, and the first collection step of collecting the cells expressing the first CAR library that accumulate in a tissue expressing a target antigen in the animal as cells expressing CARs specific to the target antigen, and there is no particular limitation on the other steps and conditions. The descriptions of the CAR library, the first screening method, and the CAR library cells of the present invention can be applied to the third scFv-manufacturing method of the present invention.

Cells expressing CARs that include scFvs capable of binding to the target antigen out of the cells expressing the CAR library are activated in a target antigen-dependent manner in the animal. The cells expressing the CAR library that have been activated move to and accumulate at a site expressing the target antigen in the animal. Accordingly, with the manufacturing method of the present invention, scFvs that can be functional in CAR-T cells and can also be involved in local invasion can be manufactured by collecting cells expressing the first CAR library that accumulate in a tissue expressing the target antigen in the animal as cells expressing CARs specific to the target antigen. The cells are activated in a target antigen-dependent manner, and therefore, it can be said that the scFvs in the cells that express the CAR library and accumulate in a tissue expressing the target antigen are more likely to recognize the target antigen. Therefore, it can be said that the manufacturing method of the present invention can be used to manufacture scFvs that are capable of binding to the target antigen and activating immune cells such as T cells, for example.

Furthermore, in the first CAR library used in the manufacturing method of the present invention, each of the first CARs is a protein that includes the first antigen-binding domain, the first transmembrane domain, and the first intracellular signaling domain. The first CAR has a structure similar to that of a CAR that includes an extracellular domain capable of binding to an antigen, a transmembrane domain, and an intracellular signaling domain, for example, and the extracellular domain capable of binding to an antigen is changed to a first antigen-binding domain to be screened for the ability to bind to the target antigen. Therefore, with the manufacturing method of the present invention, new scFvs capable of binding to the target antigen and activating immune cells such as T cells can be screened.

In the third scFv-manufacturing method of the present invention, cells expressing the first CAR library (also referred to as “first expressing cells” hereinafter) can be prepared by introducing, into cells, the first CAR library that includes nucleic acids coding for the first CARs, for example. Accordingly, the third scFv-manufacturing method of the present invention may include a first production step of producing the first CAR library prior to the first administration step. Also, the third scFv-manufacturing method of the present invention may include a first preparation step of preparing cells expressing the first CAR library prior to the first administration step by introducing the first CAR library into cells.

In the first production step, the first CAR library is produced. As described above, the first CAR library includes nucleic acids coding for the first CARs. The nucleic acids coding for the first CARs are nucleic acids (polynucleotides) coding for the amino acid sequences of the first CARs, for example. The first CAR library is the same as the first CAR library in the CAR library of the present invention, and the descriptions thereof can be applied.

The descriptions of the CAR library of the present invention can be applied to the target antigen of the first scFvs, for example.

In the first preparation step, cells expressing the first CAR library are prepared by introducing the first CAR library into cells. Specifically, in the first preparation step, the first CAR library is introduced into the above-mentioned cells, for example. In the first preparation step, the first CAR library is expressed in the cells by culturing the cells into which the first CAR library has been introduced, for example. There is no particular limitation on a method for introducing the first CAR library, and a known method for introducing nucleic acids into cells can be used, for example. Specific examples of the method for introducing the first CAR library include a method in which a nucleic acid introducing reagent such as a liposome or a cationic lipid is used; and a method in which virus such as retrovirus or lentivirus is used. The cells are cultured for 6 hours to 30 days, 6 to 96 hours, or 1 to 30 days, for example, but there is no particular limitation thereto. The cells are cultured at a temperature of 28 to 37° C., for example.

The first CAR library may include nucleic acids coding for the first CARs, or expression vectors into which nucleic acids coding for the first CARs have been introduced, for example.

Examples of the cells include, but are not particularly limited to, mammalian-derived cultured cells such as CHO cells, Jurkat cells, and Jurkat 76 cells, and immune cells. Examples of the immune cells include, but are not particularly limited to, T cells, NK cells, NKT cells, and B cells. Examples of the immune cells include cells isolated from a living organism, immune cells induced from stem cells such as multipotent stem cells, and cultured cells derived from immune cells. The isolated immune cells may be immune cell-like cultured cells, for example, and specific examples thereof include T cell-like cultured cells, NK cell-like cultured cells, NKT cell-like cultured cells, and B cell-like cultured cells. The immune cells are immune cells isolated from a living organism, for example, and specific examples thereof include immune cells derived from human peripheral blood. Examples of the T cell-like cultured cells include Jurkat cells and Jurkat 76 cells. The cells are preferably T cells or T cell-like cultured cells because scFvs that are more likely to be functional in CAR-T cells can be screened, for example.

Next, in the first administration step, the cells expressing the first CAR library are administered to an animal. In the first administration step, it is preferable to administer a plurality of types of cells expressing the first CAR library. In this case, a mixture of a plurality of types of expressing cells is used as the cells expressing the first CAR library, for example. It is preferable that some or all of the plurality of types of expressing cells code for different first CARs, and preferably code for different first antigen-binding domains, for example. When the plurality of types of expressing cells code for different first antigen-binding domains, the regions other than the first antigen-binding domains in the first CARs have the same amino acid sequence or different amino acid sequences, for example. It can also be said that the types of cells expressing the first CAR library correspond to the types of first CARs included in the cells expressing the first CAR library, for example. The number of types of first CARs included in the cells expressing the first CAR library is 1×10⁵ to 1×10⁷, 1×10⁵ to 1×10⁶, or 1×10⁶ to 5×10⁶, for example, and preferably about 2×10⁶ (e.g., 1×10⁶ to 3×10⁶). The number of types of first CARs is the number of types for 1×10⁶ cells, for example. The number of types of first CARs can also be referred to as the heterogeneity of the nucleic acids coding for the first CARs, for example. As described above, the heterogeneity can be measured through restriction enzyme mapping, sequencing of the CDRs and/or the FRHs using the Sanger's method etc., or the like, for example.

There is no particular limitation on the conditions for administrating the first expressing cells, and the administration form, the administration method, the timing of administration, the dosage amount, and the like can be determined as appropriate in accordance with the type and amount of target antigen, and the site of the animal at which the target antigen is expressed, for example.

Examples of the animal (administration target) include humans and non-human animals other than humans. Examples of the non-human animals include mammals such as mice, rats, dogs, monkeys, rabbits, sheep, horses, and pigs.

The animal is preferably an immune-suppressed animal. When the animal is a mouse, examples of the immune-suppressed mouse include nude mice, SCID (severe combined immunodeficiency) mice, and NOG (NOD/Shi-scid, IL-2Rγ KO) mice.

The administration method is not particularly limited and can be determined as appropriate in accordance with the administration target, for example. Examples of the administration method include parenteral administration and oral administration. Examples of the parenteral administration include topical administration, subcutaneous administration, intracutaneous administration, intramuscular administration, intraperitoneal administration, intravenous administration, intralymphatic administration, and intratumoral administration. The administration method is preferably intravenous administration or intralymphatic administration.

There is no particular limitation on the administration conditions of the first expressing cells. When the administration method is intravenous administration, the administration conditions of the first expressing cells are as follows: the dose (total) for a mouse is 1×10⁶ to 3×10¹⁰ cells, and preferably 1×10⁶ to 1×10⁸ cells, 1×10⁶ to 1×10⁷ cells, or about 5×10⁶ cells, for example. The administration frequency of the first expressing cells is once a week to once every four weeks, for example.

The animal may express the target antigen intrinsically or extrinsically, for example. In the latter case, the expression in the animal may be achieved by introducing a nucleic acid or expression vector coding for the target antigen or introducing cells expressing the target antigen, for example.

When the cells expressing the target antigen are introduced, the third scFv-manufacturing method of the present invention may include a first formation step of forming a tissue expressing a target antigen prior to the first administration step by introducing cells expressing the target antigen into the animal. The cells to be introduced may be cells expressing a target antigen or cells that express a target antigen after differentiation or under specific conditions, for example. Examples of the cells that express a target antigen under specific conditions include cells in which the expression of a target antigen is induced by treatment with an inducing substance such as doxycycline. The type of tissue formed by the introduced cells is not particularly limited and can be determined as appropriate in accordance with the target antigen. One or two or more types of tissues may be formed. In the latter case, the introduced cells may form an organ. In a specific example, when the target antigen is a tumor antigen, tumor cells expressing the target antigen are introduced into the animal to form a tumor tissue expressing the target antigen in the first formation step. In the present invention, the term “tissue” refers to a cell aggregate, for example. One or two or more types of cells may be included in the aggregate.

The third scFv-manufacturing method of the present invention includes a first stimulation step of administering the target antigen to the animal after the first administration step, for example. Since the third scFv-manufacturing method of the present invention includes the first stimulation step, cells expressing CARs specific to the target antigen out of the first expressing cells can be primed, thus making it possible to allow the cells expressing CARs specific to the target antigen to accumulate in a tissue expressing the target antigen. The first stimulation step is performed before the first collection step, which will be described later, after the first administration step, for example.

The first stimulation step can be performed in the same manner as the immunization of an animal with the target antigen, for example. Specifically, the first stimulation step can be performed by administering the target antigen, a nucleic acid molecule coding for the target antigen, or cells expressing the target antigen to the animal. An immunostimulant (adjuvant) may be administered together with the target antigen, for example.

Next, in the first collection step, the cells expressing the first CAR library that have accumulated in a tissue expressing the target antigen in the animal are collected as cells expressing CARs specific to the target antigen. In the first collection step, the first expressing cells that have accumulated in the tissue may be collected, or the tissue in which the first expressing cells have accumulated is collected, followed by collection of the first expressing cells from the tissue. As described above, this step may be performed on one or two or more types of tissues. In the latter case, it is preferable to collect the first expressing cells that have accumulated in two or more types of tissues in the first collection step. In this case, in the first collection step, two or more types of tissues in which the first expressing cells have accumulated may be collected, followed by collection of the first expressing cells from the two or more types of tissues. When the tissue is collected in the first collection step, a portion or all of the tissue is collected. In the first collection step, it may be confirmed if the first expressing cells accumulate in the tissue, prior to the collection of the first expressing cells. The above-mentioned confirmation can be performed using a microscope capable of live imaging, such as a two-photon excitation microscope, for example. When the confirmation is performed, it is preferable that the first expressing cells express luciferase such as SLR. Moreover, when the confirmation is performed, the first expressing cells may be labeled with a labeling substance such as a fluorescent protein prior to the confirmation, for example.

The first collection step may be performed after a desired period of time has elapsed since the first administration step was finished. Examples of the period from the completion of the first administration step to the start of the first collection step include 8 hours to 1 month, 1 day to 2 weeks, and 1 day to 1 week. When the first collection step is performed subsequently to the first stimulation step, the period from the completion of the first administration step to the start of the first collection step is a time that elapses before cells expressing CARs specific to the target antigen are activated and accumulate, for example, and specific examples thereof include 1 day to 2 weeks and 1 day to 1 week.

It is preferable that the third scFv-manufacturing method of the present invention includes a first restimulation step of restimulating cells expressing CARs specific to the target antigen with the target antigen after the first collection step. With this configuration, in the first restimulation step, it is possible to cause the proliferation of cells expressing CARs specific to the target antigen out of the cells expressing the first CAR library that have accumulated in a tissue expressing the target antigen. Accordingly, if the third scFv-manufacturing method of the present invention includes the first restimulation step, cells expressing CARs specific to the target antigen can be enriched, and the amino acid sequences of the first heavy-chain variable regions and the first light-chain variable regions of the cells expressing CARs specific to the target antigen, and the base sequences coding for the first heavy-chain variable regions and the first light-chain variable regions can be easily determined, for example.

In the first restimulation step, there is no particular limitation on the method of restimulation with the target antigen, and the restimulation can be performed by causing the cells expressing the first CAR library that have been collected from the tissue to coexist with the target antigen, for example. When the above-mentioned cells are caused to coexist with the target antigen, the target antigen may exist in the form of a monomer, a polymer such as a dimer or trimer, or a cell, for example. When the target antigen exists in the form of a cell, the cell is a cell expressing the target antigen.

The third scFv-manufacturing method of the present invention may include a first sequence determination step of determining the amino acid sequences of the first heavy-chain variable regions and the first light-chain variable regions of the first expressing cells collected in the first collection step, for example. The amino acid sequences of the first heavy-chain variable regions and the first light-chain variable regions can be determined by determining the base sequences coding for the amino acid sequences of the first heavy-chain variable regions and the first light-chain variable regions, for example. Specifically, the first sequence determination step can be performed by extracting nucleic acids from the first expressing cells collected in the first collection step, amplifying the base sequences coding for the first heavy-chain variable regions and the first light-chain variable regions in the nucleic acids, and determining the base sequences. Accordingly, with the third scFv-manufacturing method of the present invention, the amino acid sequence of the first heavy-chain variable region or the first light-chain variable region derived from the first B cell receptor can be determined, for example.

Furthermore, in the first sequence determination step, the CDRH1, the CDRH2, and the CDRH3 in each of the first heavy-chain variable regions and the CDRL1, the CDRL2, and the CDRL3 in each of the first light-chain variable regions may be identified, for example. The CDRs can be identified using a known method referring to the genome information (e.g., the website of the IMGT (http://www.imgt.org/)), for example.

The third scFv-manufacturing method of the present invention may be configured such that the first heavy-chain variable regions or first light-chain variable regions derived from the first B cell receptors in the first expressing cells collected in the first collection step are considered as the heavy-chain variable regions or light-chain variable regions of antigens and the like capable of binding to the target antigen in the first CAR library, and then the other regions are screened, for example. That is, the third scFv-manufacturing method of the present invention may be configured such that first heavy-chain variable regions or first light-chain variable regions that are newly identified in the first scFvs screened using the first CAR library (first scFvs of the first expressing cells collected in the first collection step) are used to screen the other regions. In this case, the third scFv-manufacturing method of the present invention may also be configured such that cells expressing a second CAR library are prepared based on the first heavy-chain variable regions or first light-chain variable regions derived from the first B cell receptors in the first expressing cells collected in the first collection step, and then steps similar to the first administration step and the first collection step are performed, for example. In this case, the third scFv-manufacturing method of the present invention includes a second administration step of administering cells expressing the second CAR library to an animal, and a second collection step of collecting the cells expressing the second CAR library that have accumulated in a tissue expressing a target antigen in the animal as cells expressing CARs specific to the target antigen, for example.

The second CAR library includes nucleic acids coding for second CARs, for example. Each of the second CARs includes a second antigen-binding domain, a second transmembrane domain, and a second intracellular signaling domain, for example. The second antigen-binding domain includes a second scFv to be screened for the ability to bind to the target antigen, for example.

The nucleic acids coding for the second CARs are nucleic acids (polynucleotides) coding for the amino acid sequences of the second CARs, for example.

The target antigen of the second scFvs is the same as the target antigen of the first scFvs. The second scFvs have structures similar to those of the first scFvs, for example.

Each of the second scFvs includes a second heavy-chain variable region and a second light-chain variable region, for example. The second heavy-chain variable region includes a CDRH1, a CDRH2, and a CDRH3, for example. The second light-chain variable region includes a CDRL1, a CDRL2, and a CDRL3, for example. The second heavy-chain variable region and the second light-chain variable region meet Condition 3 or Condition 4 below, for example.

### Condition 3

If the first CAR library meets Condition 1 above,
the CDRH1, the CDRH2, and the CDRH3 in the second heavy-chain variable region include the CDRH1, the CDRH2, and the CDRH3 in the heavy-chain variable region of a second B cell receptor, respectively, and
the CDRL1, the CDRL2, and the CDRL3 in the second light-chain variable region include the CDRL1, the CDRL2, and the CDRL3 in the light-chain variable region of the scFv of a cell expressing a CAR specific to the target antigen, respectively.

### Condition 4

If the first CAR library meets Condition 2 above,
the CDRH1, the CDRH2, and the CDRH3 in the second heavy-chain variable region include the CDRH1, the CDRH2, and the CDRH3 in the heavy-chain variable region of the scFv of a cell expressing a CAR specific to the target antigen, respectively, and
the CDRL1, the CDRL2, and the CDRL3 in the second light-chain variable region include the CDRL1, the CDRL2, and the CDRL3 in the light-chain variable region of a second B cell receptor, respectively.

In the third scFv-manufacturing method of the present invention, cells expressing the second CAR library (also referred to as “second expressing cells” hereinafter) can be prepared by introducing, into cells, the second CAR library that includes nucleic acids coding for the second CARs, for example. Accordingly, the third scFv-manufacturing method of the present invention may include a second production step of producing the second CAR library prior to the second administration step. Also, the third scFv-manufacturing method of the present invention may include a second preparation step of preparing cells expressing the second CAR library prior to the second administration step by introducing the second CAR library into cells.

In the second production step, the second CAR library is produced. As described above, the second CAR library includes nucleic acids coding for the second CARs. The nucleic acids coding for the second CARs are nucleic acids (polynucleotides) coding for the amino acid sequences of the second CARs, for example. The second CAR library is the same as the second CAR library in the CAR library of the present invention, and the descriptions thereof can be applied.

As described above, the second heavy-chain variable region includes the CDRH1, the CDRH2, and the CDRH3. The second light-chain variable region includes the CDRL1, the CDRL2, and the CDRL3. The second heavy-chain variable region and the second light-chain variable region meet Condition 3 or Condition 4 above.

Condition 3 above is employed in the case where the second CAR library is prepared using the first light-chain variable regions screened using the first CAR library that meets Condition 1 above, for example. In Condition 3 above, the CDRL1, the CDRL2, and the CDRL3 of the first light-chain variable region screened using the first CAR library are used as those in the second light-chain variable region, and the CDRH1, the CDRH2, and the CDRH3 in the second heavy-chain variable region are screened for the ability to bind to the target antigen, for example.

Next, Condition 4 above is employed in the case where the second CAR library is prepared using the heavy-chain variable regions screened using the first CAR library that meets Condition 2 above, for example. In Condition 4 above, the CDRH1, the CDRH2, and the CDRH3 of the heavy-chain variable region screened using the first CAR library are used as those in the second heavy-chain variable region, and the CDRL1, the CDRL2, and the CDRL3 in the second light-chain variable region are screened for the ability to bind to the target antigen, for example.

Next, in the second preparation step, cells expressing the second CAR library are prepared by introducing the second CAR library into cells. Specifically, in the second preparation step, the second CAR library is introduced into the above-mentioned cells, for example. In the second preparation step, the second CAR library is expressed in the cells by culturing the cells into which the second CAR library has been introduced, for example. The descriptions of the method for introducing the first CAR library can be applied to the method for introducing the second CAR library, for example.

The second CAR library may include nucleic acids coding for the second CARs, or expression vectors into which nucleic acids coding for the second CARs have been introduced, for example.

There is no particular limitation on the cells, and the descriptions of the cells into which the first CAR library is to be introduced can be applied, for example.

Next, in the second administration step, the cells expressing the second CAR library are administered to an animal. In the second administration step, it is preferable to administer a plurality of types of cells expressing the second CAR library. In this case, a mixture of a plurality of types of expressing cells is used as the cells expressing the second CAR library, for example. It is preferable that some or all of the plurality of types of expressing cells code for different second CARs, and preferably code for different second antigen-binding domains, for example. When the plurality of types of expressing cells code for different second antigen-binding domains, the regions other than the second antigen-binding domains in the second CARs have the same amino acid sequence or different amino acid sequences, for example. It can also be said that the types of cells expressing the second CAR library correspond to the types of second CARs included in the cells expressing the second CAR library, for example. The number of types of second CARs included in the cells expressing the second CAR library is 1×10⁵ to 1×10⁷, 1×10⁵ to 1×10⁶, or 1×10⁶ to 5×10⁶, for example, and preferably about 2×10⁶ (e.g., 1×10⁶ to 3×10⁶). The number of types of second CARs can also be referred to as the heterogeneity of the nucleic acids coding for the second CARs, for example. As described above, the heterogeneity can be measured through restriction enzyme mapping, sequencing of the CDRs and/or the FRHs using the Sanger's method etc., or the like, for example.

There is no particular limitation on the administration conditions of the second expressing cells, and the descriptions of the above-mentioned administration conditions of the second expressing cells can be applied, for example.

In the second administration step, the animal may express the target antigen intrinsically or extrinsically, for example. In the latter case, the expression in the animal may be achieved by introducing a nucleic acid or expression vector coding for the target antigen or introducing cells expressing the target antigen, for example.

When the cells expressing the target antigen are introduced, the third scFv-manufacturing method of the present invention may include a second formation step of forming a tissue expressing a target antigen prior to the second administration step by introducing cells expressing the target antigen into the animal. The cells to be introduced may be cells expressing a target antigen or cells that express a target antigen after differentiation or under specific conditions, for example. Examples of the cells that express a target antigen under specific conditions include cells in which the expression of a target antigen is induced by treatment with an inducing substance such as doxycycline. The type of tissue formed by the introduced cells is not particularly limited and can be determined as appropriate in accordance with the target antigen. One or two or more types of tissues may be formed. In the latter case, the introduced cells may form an organ. In a specific example, when the target antigen is a tumor antigen, tumor cells expressing the target antigen are introduced into the animal to form a tumor tissue expressing the target antigen in the second formation step.

The third scFv-manufacturing method of the present invention includes a second stimulation step of administering the target antigen to the animal after the second administration step, for example. Since the third scFv-manufacturing method of the present invention includes the second stimulation step, cells expressing CARs specific to the target antigen out of the second expressing cells can be primed, thus making it possible to allow the cells expressing CARs specific to the target antigen to accumulate in a tissue expressing the target antigen. The second stimulation step is performed in the second collection step, which will be described later, after the second administration step, for example.

The second stimulation step can be performed in the same manner as the immunization of an animal with the target antigen, for example. Specifically, the second stimulation step can be performed by administering the target antigen, a nucleic acid molecule coding for the target antigen, or cells expressing the target antigen to the animal. An immunostimulant (adjuvant) may be administered together with the target antigen, for example.

Next, in the second collection step, the cells expressing the second CAR library that have accumulated in a tissue expressing the target antigen in the animal are collected as cells expressing CARs specific to the target antigen. In the second collection step, the second expressing cells that have accumulated in the tissue may be collected, or the tissue in which the second expressing cells have accumulated is collected, followed by collection of the second expressing cells from the tissue. As described above, this step may be performed on one or two or more types of tissues. In the latter case, it is preferable to collect the second expressing cells that have accumulated in two or more types of tissues in the second collection step. In this case, in the second collection step, two or more types of tissues in which the second expressing cells have accumulated may be collected, followed by collection of the second expressing cells from the two or more types of tissues. When the tissue is collected in the second collection step, a portion or all of the tissue is collected. In the second collection step, it may be confirmed if the second expressing cells accumulate in the tissue, prior to the collection of the second expressing cells. The above-mentioned confirmation can be performed using a microscope capable of live imaging, such as a two-photon excitation microscope, for example. When the confirmation is performed, it is preferable that the second expressing cells express luciferase such as SLR. Moreover, when the confirmation is performed, the second expressing cells may be labeled with a labeling substance such as a fluorescent protein prior to the confirmation, for example.

The second collection step may be performed after a desired period of time has elapsed since the second administration step was finished. Examples of the period from the completion of the second administration step to the start of the second collection step include 8 hours to 1 month, 1 day to 2 weeks, and 1 day to 1 week. When the second collection step is performed subsequently to the second stimulation step, the period from the completion of the second administration step to the start of the second collection step is a time that elapses before cells expressing CARs specific to the target antigen are activated and accumulate, for example, and specific examples thereof include 1 day to 2 weeks and 1 day to 1 week.

It is preferable that the third scFv-manufacturing method of the present invention includes a second restimulation step of restimulating cells expressing CARs specific to the target antigen with the target antigen after the second collection step. With this configuration, in the second restimulation step, it is possible to cause the proliferation of cells expressing CARs specific to the target antigen out of the cells expressing the second CAR library that have accumulated in a tissue expressing the target antigen. Accordingly, if the third scFv manufacturing method of the present invention includes the second restimulation step, cells expressing CARs specific to the target antigen can be enriched, and the amino acid sequences of the second heavy-chain variable regions and the second light-chain variable regions of the cells expressing CARs specific to the target antigen, and the base sequences coding for the second heavy-chain variable regions and the second light-chain variable regions can be easily determined, for example.

In the second restimulation step, there is no particular limitation on the method of restimulation with the target antigen, and if the “first CAR library” is changed to the “second CAR library”, the descriptions of the first restimulation step can be applied, for example.

The third scFv-manufacturing method of the present invention may include a second sequence determination step of determining the amino acid sequences of the second heavy-chain variable regions and the second light-chain variable regions of the second expressing cells collected in the second collection step, for example. The amino acid sequences of the second heavy-chain variable regions and the second light-chain variable regions can be determined by determining the base sequences coding for the amino acid sequences of the second heavy-chain variable regions and the second light-chain variable regions, for example. Specifically, the second sequence determination step can be performed by extracting nucleic acids from the second expressing cells collected in the second collection step, amplifying the base sequences coding for the second heavy-chain variable regions and the second light-chain variable regions in the nucleic acids, and determining the base sequences. Accordingly, with the third scFv-manufacturing method of the present invention, the amino acid sequence of the second heavy-chain variable region or the second light-chain variable region derived from the second B cell receptor can be determined, for example.

Furthermore, in the second sequence determination step, the CDRH1, the CDRH2, and the CDRH3 in each of the second heavy-chain variable regions and the CDRL1, the CDRL2, and the CDRL3 in each of the second light-chain variable regions may be identified, for example. The CDRs can be identified using a known method referring to the genome information (e.g., the website of the IMGT (http://www.imgt.org/)), for example.

### EXAMPLES

Next, examples of the present invention will be described. However, the present invention is not limited to the examples below. Commercially available reagents were used according to their protocols unless otherwise stated.

### Example 1

It was confirmed that the screening method of the present invention in which the CAR library of the present invention is used could be used to screen scFvs capable of binding to A2/NY-ESO-1₁₅₇.

### (1) Preparation of 3M4E5 CAR

A 3M4E5 CAR was prepared using an A2/NY-ESO-1₁₅₇-specific antibody (clone: 3M4E5). Specifically, a nucleic acid coding for a 3M4E5 scFv was prepared by coupling a nucleic acid (Sequence ID No. 162) coding for the heavy-chain variable region derived from the immunoglobulin heavy chain of 3M4E5 and a nucleic acid (Sequence ID No. 163) coding for the light-chain variable region derived from the immunoglobulin light chain of 3M4E5 to each other via a nucleic acid (Sequence ID No. 3) coding for an Fv linker peptide (GSTSGSGKPGSGEGSTKG: Sequence ID No. 1). An scFv obtained by coupling the heavy-chain variable region of 3M4E5, the Fv linker peptide, and the light-chain variable region of 3M4E5 in this order is referred to as a “3M4E5HL scFv”. An scFv obtained by coupling the light-chain variable region of 3M4E5, the Fv linker peptide, and the heavy-chain variable region of 3M4E5 in this order is referred to as a “3M4E5LH scFv”.

Nucleic Acid Coding for Heavy-Chain Variable
Region Derived from Immunoglobulin Heavy
Chain of 3M4E5
(Sequence ID No. 162)
5′-
GAAGTGCAGCTGCTGGAATCTGGCGGCGGACTGGTGCAGCCTGGCGG
ATCTCTGAGACTGAGCTGTGCCGCCAGCGGCTTCACCTTCAGCACCT
ACCAGATGAGCTGGGTGCGCCAGGCCCCTGGCAAAGGACTGGAATGG
GTGTCCGGCATCGTGTCCAGCGGCGGCTCTACAGCCTACGCCGATAG
CGTGAAGGGCCGGTTCACCATCAGCCGGGACAACAGCAAGAACACCC
TGTACCTGCAGATGAACAGCCTGAGAGCCGAGGACACCGCCGTGTAC
TATTGTGCCGGGGAGCTGCTGCCCTACTACGGCATGGATGTGTGGGG
CCAGGGCACCACCGTGACAGTGTCCTCA-3′
Nucleic Acid Coding for Light-Chain Variable
Region Derived from Immunoglobulin Light
Chain of 3M4E5
(Sequence ID No. 163)
5′-
CAGAGCGAGCTGACACAGCCTAGATCCGTGTCTGGCAGCCCTGGCCA
GAGCGTGACCATCAGCTGTACCGGCACCAGCAGAGATGTGGGCGGCT
ACAACTACGTGTCCTGGTATCAGCAGCATCCCGGCAAGGCCCCCAAG
CTGATCATCCACGACGTGATCGAGCGGAGCAGCGGCGTGCCCGATAG
ATTCAGCGGCAGCAAGAGCGGCAACACCGCCAGCCTGACAATCAGCG
GACTGCAGGCCGAGGACGAGGCCGACTACTACTGTTGGAGCTTCGCC
GGCAGCTACTACGTGTTCGGCACCGGCACCGATGTGACCGTGCTG-3′
3M4E5HL scFv
(Sequence ID No. 164)
EVQLLESGGGLVQPGGSLRLSCAASGFTFSTYQMSWVRQAPGKGLEW
VSGIVSSGGSTAYADSVKGRFTISRDNSKNTLYLQMNSLRAEDTAVY
YCAGELLPYYGMDVWGQGTTVTVSSGSTSGSGKPGSGEGSTKGQSEL
TQPRSVSGSPGQSVTISCTGTSRDVGGYNYVSWYQQHPGKAPKLIIH
DVIERSSGVPDRFSGSKSGNTASLTISGLQAEDEADYYCWSFAGSYY
VFGTGTDVTVL
Nucleic Acid Coding for 3M4E5HL scFv
(Sequence ID No. 165)
5′-
GAAGTGCAGCTGCTGGAATCTGGCGGCGGACTGGTGCAGCCTGGCGG
ATCTCTGAGACTGAGCTGTGCCGCCAGCGGCTTCACCTTCAGCACCT
ACCAGATGAGCTGGGTGCGCCAGGCCCCTGGCAAAGGACTGGAATGG
GTGTCCGGCATCGTGTCCAGCGGCGGCTCTACAGCCTACGCCGATAG
CGTGAAGGGCCGGTTCACCATCAGCCGGGACAACAGCAAGAACACCC
TGTACCTGCAGATGAACAGCCTGAGAGCCGAGGACACCGCCGTGTAC
TATTGTGCCGGGGAGCTGCTGCCCTACTACGGCATGGATGTGTGGGG
CCAGGGCACCACCGTGACAGTGTCCTCAGGCTCTACAAGCGGCTCTG
GCAAGCCTGGATCTGGCGAGGGAAGCACCAAGGGCCAGAGCGAGCTG
ACACAGCCTAGATCCGTGTCTGGCAGCCCTGGCCAGAGCGTGACCAT
CAGCTGTACCGGCACCAGCAGAGATGTGGGCGGCTACAACTACGTGT
CCTGGTATCAGCAGCATCCCGGCAAGGCCCCCAAGCTGATCATCCAC
GACGTGATCGAGCGGAGCAGCGGCGTGCCCGATAGATTCAGCGGCAG
CAAGAGCGGCAACACCGCCAGCCTGACAATCAGCGGACTGCAGGCCG
AGGACGAGGCCGACTACTACTGTTGGAGCTTCGCCGGCAGCTACTAC
GTGTTCGGCACCGGCACCGATGTGACCGTGCTG-3′
3M4E5LH scFv
(Sequence ID No. 166)
QSELTQPRSVSGSPGQSVTISCTGTSRDVGGYNYVSWYQQHPGKAPK
LIIHDVIERSSGVPDRFSGSKSGNTASLTISGLQAEDEADYYCWSFA
GSYYVFGTGTDVTVLGSTSGSGKPGSGEGSTKGEVQLLESGGGLVQP
GGSLRLSCAASGFTFSTYQMSWVRQAPGKGLEWVSGIVSSGGSTAYA
DSVKGRFTISRDNSKNTLYLQMNSLRAEDTAVYYCAGELLPYYGMDV
WGQGTTVTVSS
Nucleic Acid Coding for 3M4E5LH scFv
(Sequence ID No. 167)
5′-
CAGAGCGAGCTGACACAGCCTAGATCCGTGTCTGGCAGCCCTGGCCA
GAGCGTGACCATCAGCTGTACCGGCACCAGCAGAGATGTGGGCGGCT
ACAACTACGTGTCCTGGTATCAGCAGCATCCCGGCAAGGCCCCCAAG
CTGATCATCCACGACGTGATCGAGCGGAGCAGCGGCGTGCCCGATAG
ATTCAGCGGCAGCAAGAGCGGCAACACCGCCAGCCTGACAATCAGCG
GACTGCAGGCCGAGGACGAGGCCGACTACTACTGTTGGAGCTTCGCC
GGCAGCTACTACGTGTTCGGCACCGGCACCGATGTGACCGTGCTGGG
CTCTACAAGCGGCTCTGGCAAGCCTGGATCTGGCGAGGGAAGCACCA
AGGGCGAAGTGCAGCTGCTGGAATCTGGCGGCGGACTGGTGCAGCCT
GGCGGATCTCTGAGACTGAGCTGTGCCGCCAGCGGCTTCACCTTCAG
CACCTACCAGATGAGCTGGGTGCGCCAGGCCCCTGGCAAAGGACTGG
AATGGGTGTCCGGCATCGTGTCCAGCGGCGGCTCTACAGCCTACGCC
GATAGCGTGAAGGGCCGGTTCACCATCAGCCGGGACAACAGCAAGAA
CACCCTGTACCTGCAGATGAACAGCCTGAGAGCCGAGGACACCGCCG
TGTACTATTGTGCCGGGGAGCTGCTGCCCTACTACGGCATGGATGTG
TGGGGCCAGGGCACCACCGTGACAGTGTCCTCA-3′

Next, a nucleic acid (Sequence ID No. 169) coding for a sequence (Sequence ID No. 168) that included a partial region, the transmembrane domain, and the intracellular signaling domain of human CD28, and a nucleic acid coding for the intracellular signaling domain of human CD3ζ were coupled to the 3′ ends of the nucleic acids coding for the 3M4E5HL scFv and the 3M4E5LH scFv, such that these nucleic acids were arranged in the stated order from the 5′ end side. Thus, a nucleic acid coding for a 3M4E5HL CAR that included the 3M4E5HL scFv and a nucleic acid coding for a 3M4E5LH CAR that included the 3M4E5LH scFv were prepared. It should be noted that the nucleic acid coding for the partial region, the transmembrane domain, and the intracellular signaling domain of human CD28 and the nucleic acid coding for the intracellular signaling domain of human CD3ζ were obtained using the GeneArt (trademark) artificial gene synthesis service (provided by Thermo Fisher Scientific). A NotI restriction enzyme site was added to the 5′ end of the nucleic acid coding for the partial region, the transmembrane domain, and the intracellular signaling domain of human CD28.

Partial Region, Transmembrane Domain,
Intracellular Signaling Domain of Human CD28
(Sequence ID No. 168)
AAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRK
HYQPYAPPRDFAAYRS
Nucleic Acid Coding for Partial Region,
Transmembrane Domain, Intracellular Signaling
Domain of Human CD28
(Sequence ID No. 169)
5′-
GCGGCCGCAATCGAAGTGATGTACCCCCCTCCCTACCTGGACAACGA
GAAGTCCAACGGCACCATTATCCACGTGAAGGGAAAGCACCTGTGCC
CCAGCCCTCTGTTCCCTGGCCCTAGCAAGCCTTTCTGGGTGCTGGTG
GTCGTGGGCGGAGTGCTGGCCTGTTATAGCCTGCTCGTGACCGTGGC
CTTCATCATCTTTTGGGTGCGCAGCAAGCGGAGCCGGCTGCTGCACA
GCGACTACATGAACATGACCCCCAGACGGCCCGGACCCACCAGAAAG
CACTACCAGCCTTACGCCCCTCCCAGAGACTTCGCCGCCTACAGATC
T-3′
Intracellular Signaling Domain of Human CD3ζ
(Sequence ID No. 170)
RVKFSRSADAPAYQQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPR
Nucleic Acid Coding for Intracellular
Signaling Domain of Human CD3ζ
(Sequence ID No. 171)
5′-
CGAGTGAAGTTCAGCAGAAGCGCCGACGCCCCTGCCTATCAGCAGGG
CCAGAACCAGCTGTACAACGAGCTGAACCTGGGCAGACGGGAAGAGT
ACGACGTGCTGGACAAGCGGAGAGGCAGGGACCCTGAGATGGGCGGC
AAGCCCAGAAGAAAGAACCCCCAGGAAGGCCTGTATAACGAACTGCA
GAAAGACAAGATGGCCGAGGCCTACAGCGAGATCGGCATGAAGGGCG
AGCGGAGAAGAGGCAAGGGCCACGATGGCCTGTACCAGGGCCTGAGC
ACCGCCACCAAGGACACCTATGACGCCCTGCACATGCAGGCCCTGCC
CCCCAGA-3′

Furthermore, a nucleic acid coding for the truncated-NGFR gene (ΔNGFR) was coupled to the 3′ ends of the nucleic acid coding for the 3M4E5HL CAR and the nucleic acid coding for the 3M4E5LH CAR via a nucleic acid coding for a furin cleavage site (RAKR: Sequence ID No. 172), a spacer sequence (SGSG: Sequence ID No. 173), and a codon-optimized P2A sequence (ATNFSLLKQAGDVEENPGP: Sequence ID No. 174). Thus, a nucleic acid coding for a tagged 3M4E5HL CAR and a nucleic acid coding for a tagged 3M4E5LH CAR were prepared. The nucleic acids were introduced into pMX expression vectors.

### (2) Preparation of First CAR Library

A B-cell purification kit (CD19-positive selection kit, MACS (registered trademark) beads, manufactured by Miltenyi Biotec) was used to purify 1×10⁶ to 3×10⁶ human peripheral blood B cells from human peripheral blood monocytes. After the obtained CD19⁺ B cells were collected, RNA was extracted using a cDNA synthesis kit (SMARTer (trademark) RACE cDNA amplification kit, manufactured by Takara Bio), and then cDNA was synthesized from the RNA.

A nucleic acid coding for a protein that included the heavy-chain variable region and a portion of the constant region derived from the immunoglobulin heavy chain was amplified through PCR in which the above-mentioned cDNA and a mixture of a 5′-RACE primer, a 3′IGHC primer, and a 3′IGHM primer were used. The 3′IGHC primer is capable of hybridizing with the CH1 region of a human IgG. The 3′IGHM primer is capable of hybridizing with the CH1 region of a human IgM.

5′-RACE Primer
(Sequence ID No. 175)
5′-CTAATACGACTCACTATAGGGCAAGCAGTGGTATCAACGCAGA
GT-3′
3′IGHC Primer
(Sequence ID No. 176)
5′-TGAGTTCCACGACACCGTCAC-3′
3′IGHM Primer
(Sequence ID No. 177)
5′-TCCAGGACAAAGTGATGGAGTC-3′

Next, semi-nested PCR was performed in which the first PCR product and a mixture of a modified 5′-RACE primer, a 3′IGHJ1 primer, a 3′IGHJ2 primer, and a 3′IGHJ3 primer were used, and thus a nucleic acid coding for the heavy-chain variable region was amplified. Thus, a library of nucleic acids coding for the heavy-chain variable regions derived from human peripheral blood B cells was prepared. The 3′IGHJ1 primer, the 3′IGHJ2 primer, and the 3′IGHJ3 primer are capable of hybridizing with the J regions (J gene fragments) of the heavy chain.

Modified 5′-RACE Primer
(Sequence ID No. 178)
5′-GTGTGGTGGTACGGGAATTCAAGCAGTGGTATCAACGCAGAGT-3′
3′IGHJ1 Primer
(Sequence ID No. 179)
5′-CTTGCCAGAGCCGCTTGTAGAGCCTGAGGAGACGGTGACCAGGG-3′
3′IGHJ2 Primer
(Sequence ID No. 180)
5′-CTTGCCAGAGCCGCTTGTAGAGCCTGAAGAGACGGTGACCATTG
TC-3′
3′IGHJ3 Primer
(Sequence ID No. 181)
5′-CTTGCCAGAGCCGCTTGTAGAGCCTGAGGAGACGGTGACCGTGG
TC-3′

Next, a nucleic acid coding for the light-chain variable region derived from the immunoglobulin light chain was amplified in the same manner as in the above-mentioned amplification of the nucleic acid coding for the heavy-chain variable region, except that a 3′IGKC primer and a 3′IGLC primer were used in the first PCR instead of the 3′IGHC primer and the 3′IGHM primer, and a 3′IGKJ1 primer, a 3′IGKJ2 primer, a 3′IGKJ3 primer, a 3′IGKJ4 primer, a 3′IGLJ1 primer, a 3′IGLJ2 primer, a 3′IGLJ4 primer, a 3′IGLJ5 primer, and a 3′IGLJ7 primer were used in the second semi-nested PCR instead of the 3′IGHJ1 primer, the 3′IGHJ2 primer, and the 3′IGHJ3 primer. Thus, a library of nucleic acids coding for the light-chain variable regions derived from human peripheral blood B cells was prepared. The 3′IGKC primer and the 3′IGLC primer are capable of hybridizing with the CH1 regions of a κ chain and a λ chain, respectively. The 3′IGKJ1 primer, the 3′IGKJ2 primer, the 3′IGKJ3 primer, the 3′IGKJ4 primer, the 3′IGLJ1 primer, the 3′IGLJ2 primer, the 3′IGLJ4 primer, the 3′IGLJ5 primer, and the 3′IGLJ7 primer are capable of hybridizing with the J regions (J gene fragments) of the light chain.

3′IGKC Primer
(Sequence ID No. 182)
5′-AGGGTCAGAGGCCAAAGGATGG-3′
3′IGLC Primer
(Sequence ID No. 183)
5′-CTTGGAGCTCCTCAGAGGAG-3′
3′IGKJ1 Primer
(Sequence ID No. 184)
5′-CTTGCCAGAGCCGCTTGTAGAGCCTTTGATTTCCACCTTGGTCCC-3′
3′IGKJ2 Primer
(Sequence ID No. 185)
5′-CTTGCCAGAGCCGCTTGTAGAGCCTTTGATCTCCAGCTTGGTCCC-3′
3′IGKJ3 Primer
(Sequence ID No. 186)
5′-CTTGCCAGAGCCGCTTGTAGAGCCTTTGATATCCACTTTGGTCCC-3′
3′IGKJ4 Primer
(Sequence ID No. 187)
5′-CTTGCCAGAGCCGCTTGTAGAGCCTTTAATCTCCAGTCGTGTCCC-3′
3′IGLJ1 Primer
(Sequence ID No. 188)
5′-CTTGCCAGAGCCGCTTGTAGAGCCTAGGACGGTGACCTTGGTCCC-3′
3′IGLJ2 Primer
(Sequence ID No. 189)
5′-CTTGCCAGAGCCGCTTGTAGAGCCTAGGACGGTCAGCTTGGTCCC-3′
3′IGLJ4 Primer
(Sequence ID No. 190)
5′-CTTGCCAGAGCCGCTTGTAGAGCCTAAAATGATCAGCTGGGTTCC-3′
3′IGLJ5 Primer
(Sequence ID No. 191)
5′-CTTGCCAGAGCCGCTTGTAGAGCCTAGGACGGTCAGCTCGGTCCC-3′
3′IGLJ7 Primer
(Sequence ID No. 192)
5′-CTTGCCAGAGCCGCTTGTAGAGCCGAGGACGGTCAGCTGGGTGCC-3′

An hH-3M4E4L scFv library was prepared by substituting the nucleic acid coding for the heavy-chain variable region on the 5′ side of the nucleic acid coding for the 3M4E5HL scFv described in (1) above with the above-mentioned library that included the nucleic acids of the heavy-chain variable regions derived from human peripheral blood B cells. Then, a tagged hH-3M4E4L CAR library was prepared in the same manner as in (1) above, except that the hH-3M4E4L scFv library was used instead of the nucleic acid coding for the 3M4E5HL scFv. The tagged hH-3M4E4L CAR library was introduced into pMX expression vectors to prepare expression vectors for expressing the hH-3M4E4L CAR library. It should be noted that, regarding the heterogeneity of the heavy-chain variable regions in the hH-3M4E4L CAR library, it was confirmed through restriction enzyme mapping and sequencing of the regions using the Sanger's method that the number of types of heavy-chain variable regions was about 1×10⁶.

### (3) Preparation of First CAR Library-Expressing T Cells (First Candidate CAR-Ts)

The expression vectors for expressing the hH-3M4E4L CAR library were introduced into Plat-A cells (provided by Dr. Toshio KITAMURA, the Institute of Medical Science, the University of Tokyo, National University Corporation) using a transfection reagent (TransIT293, manufactured by Takara Bio), and then a culture supernatant containing ecotropic retroviruses was prepared. The Plat-A cells were maintained in a DMEM culture medium containing fetal calf serum (FCS) at a concentration of 10%, puromycin at a concentration of 1 μg/mL, and blasticidin at a concentration of 10 μg/mL until before use. Next, the hH-3M4E4L CAR library was introduced into PG13 cells serving as packaging cells by adding the above-mentioned culture supernatant containing ecotropic viruses, and thus GaLV-pseudotyped retroviruses that included the hH-3M4E4L CAR library were prepared. Then, CAR-Ts were prepared by infecting human peripheral blood T cells with the GaLV-pseudotyped retroviruses. A DMEM culture medium containing FCS at a concentration of 10% was used as the culture medium for PG13 cells, and PG13 cells were cultured under a wet atmosphere in the culture conditions of 37° C. and 5% CO₂. The same cell culture conditions were used in the descriptions below unless otherwise stated.

Next, human peripheral blood monocytes were isolated from a healthy subject. The obtained human peripheral blood monocytes were cultured in the presence of an anti-CD3 antibody (clone: OKT3) at a concentration of 50 ng/ml and human IL-2 at a concentration of 100 IU/mL for two days. An RPMI1640 culture medium containing gentamicin at a concentration of 50 μg/mL and human AB serum (manufactured by Sigma) at a concentration of 10% was used as the culture medium for the human peripheral blood monocytes. After the above-mentioned culture, T cells were centrifuged in the presence of the GaLV-pseudotyped retroviruses under the conditions of 1000×g and 32° C. for 1 hour and thus infected with the retroviruses, and thus the hH-3M4E4L CAR library was introduced into the T cells. The infection with the retroviruses and the introduction of the hH-3M4E4L CAR library were performed in the same manner five more times (six times in total). After the resulting T cells were stained with a PE-labeled anti-human NGFR mAb (clone: ME20.4), it was examined using a flow cytometer (Gallios flow cytometer, manufactured by Beckman Coulter) if the tags were expressed on the T cells, and it was thus confirmed that a single hH-3M4E4L CAR was expressed in a single T cell at an introduction efficiency of 30%. Analysis software (FlowJo Version 7.6.5 software, manufactured by TreeStar) was used to analyze data obtained using the flow cytometer (the same applies hereinafter).

The T cells into which the hH-3M4E4 CAR library had been introduced were reacted with the PE-labeled anti-human NGFR mAb. After the above-mentioned reaction, the T cells into which the hH-3M4E4 CAR library had been introduced were purified using anti-PE microbeads (manufactured by Miltenyi Biotec), and the purified T cells were used as first candidate CAR-Ts.

### (4) Preparation of Target Antigen-Expressing Cells

Retroviruses were used to introduce a nucleic acid coding for HLA-A*02:01 and nucleic acids coding for CD80, CD83, CD40, and 4-1BBL into K562 cells (HLA and CD19 are not expressed; available from ATCC), and thus antigen-presenting cells (APCs, K562/A2) were obtained.

Nucleic Acid Coding for HLA-A*02:01
(Sequence ID No. 193)
5′-ATGGCCGTCATGGCGCCCCGAACCCTCGTCCTGCTACTCTCGGGGG
CTCTGGCCCTGACCCAGACCTGGGCGGGCTCTCACTCCATGAGGTATTT
CTTCACATCCGTGTCCCGGCCCGGCCGCGGGGAGCCCCGCTTCATCGCA
GTGGGCTACGTGGACGACACGCAGTTCGTGCGGTTCGACAGCGACGCCG
CGAGCCAGAGGATGGAGCCGCGGGCGCCGTGGATAGAGCAGGAGGGTCC
GGAGTATTGGGACGGGGAGACACGGAAAGTGAAGGCCCACTCACAGACT
CACCGAGTGGACCTGGGGACCCTGCGCGGCTACTACAACCAGAGCGAGG
CCGGTTCTCACACCGTCCAGAGGATGTATGGCTGCGACGTGGGGTCGGA
CTGGCGCTTCCTCCGCGGGTACCACCAGTACGCCTACGACGGCAAGGAT
TACATCGCCCTGAAAGAGGACCTGCGCTCTTGGACCGCGGCGGACATGG
CAGCTCAGACCACCAAGCACAAGTGGGAGGCGGCCCATGTGGCGGAGCA
GTTGAGAGCCTACCTGGAGGGCACGTGCGTGGAGTGGCTCCGCAGATAC
CTGGAGAACGGGAAGGAGACGCTGCAGCGCACGGACGCCCCCAAAACGC
ATATGACTCACCACGCTGTCTCTGACCATGAAGCCACCCTGAGGTGCTG
GGCCCTGAGCTTCTACCCTGCGGAGATCACACTGACCTGGCAGCGGGAT
GGGGAGGACCAGACCCAGGACACGGAGCTCGTGGAGACCAGGCCTGCAG
GGGATGGAACCTTCCAGAAGTGGGCGGCTGTGGTGGTGCCTTCTGGACA
GGAGCAGAGATACACCTGCCATGTGCAGCATGAGGGTTTGCCCAAGCCC
CTCACCCTGAGATGGGAGCCGTCTTCCCAGCCCACCATCCCCATCGTGG
GCATCATTGCTGGCCTGGTTCTCTTTGGAGCTGTGATCACTGGAGCTGT
GGTCGCTGCTGTGATGTGGAGGAGGAAGAGCTCAGATAGAAAAGGAGGG
AGCTACTCTCAGGCTGCAAGCAGTGACAGTGCCCAGGGCTCTGATGTGT
CTCTCACAGCTTGTAAAGTG-3′
Nucleic Acid Coding for CD80
(Sequence ID No. 194)
5′-ATGGGCCACACACGGAGGCAGGGAACATCACCATCCAAGTGTCCAT
ACCTCAATTTCTTTCAGCTCTTGGTGCTGGCTGGTCTTTCTCACTTCTG
TTCAGGTGTTATCCACGTGACCAAGGAAGTGAAAGAAGTGGCAACGCTG
TCCTGTGGTCACAATGTTTCTGTTGAAGAGCTGGCACAAACTCGCATCT
ACTGGCAAAAGGAGAAGAAAATGGTGCTGACTATGATGTCTGGGGACAT
GAATATATGGCCCGAGTACAAGAACCGGACCATCTTTGATATCACTAAT
AACCTCTCCATTGTGATCCTGGCTCTGCGCCCATCTGACGAGGGCACAT
ACGAGTGTGTTGTTCTGAAGTATGAAAAAGACGCTTTCAAGCGGGAACA
CCTGGCTGAAGTGACGTTATCAGTCAAAGCTGACTTCCCTACACCTAGT
ATATCTGACTTTGAAATTCCAACTTCTAATATTAGAAGGATAATTTGCT
CAACCTCTGGAGGTTTTCCAGAGCCTCACCTCTCCTGGTTGGAAAATGG
AGAAGAATTAAATGCCATCAACACAACAGTTTCCCAAGATCCTGAAACT
GAGCTCTATGCTGTTAGCAGCAAACTGGATTTCAATATGACAACCAACC
ACAGCTTCATGTGTCTCATCAAGTATGGACATTTAAGAGTGAATCAGAC
CTTCAACTGGAATACAACCAAGCAAGAGCATTTTCCTGATAACCTGCTC
CCATCCTGGGCCATTACCTTAATCTCAGTAAATGGAATTTTTGTGATAT
GCTGCCTGACCTACTGCTTTGCCCCAAGATGCAGAGAGAGAAGGAGGAA
TGAGAGATTGAGAAGGGAAAGTGTACGCCCTGTA-3′
Nucleic Acid Coding for CD83
(Sequence ID No. 195)
5′-ATGTCGCGCGGCCTCCAGCTTCTGCTCCTGAGCTGCGCCTACAGCC
TGGCTCCCGCGACGCCGGAGGTGAAGGTGGCTTGCTCCGAAGATGTGGA
CTTGCCCTGCACCGCCCCCTGGGATCCGCAGGTTCCCTACACGGTCTCC
TGGGTCAAGTTATTGGAGGGTGGTGAAGAGAGGATGGAGACACCCCAGG
AAGACCACCTCAGGGGACAGCACTATCATCAGAAGGGGCAAAATGGTTC
TTTCGACGCCCCCAATGAAAGGCCCTATTCCCTGAAGATCCGAAACACT
ACCAGCTGCAACTCGGGGACATACAGGTGCACTCTGCAGGACCCGGATG
GGCAGAGAAACCTAAGTGGCAAGGTGATCTTGAGAGTGACAGGATGCCC
TGCACAGCGTAAAGAAGAGACTTTTAAGAAATACAGAGCGGAGATTGTC
CTGCTGCTGGCTCTGGTTATTTTCTACTTAACACTCATCATTTTCACTT
GTAAGTTTGCACGGCTACAGAGTATCTTCCCAGATTTTTCTAAAGCTGG
CATGGAACGAGCTTTTCTCCCAGTTACCTCCCCAAATAAGCATTTAGGG
CTAGTGACTCCTCACAAGACAGAACTGGTA-3′
Nucleic Acid Coding for CD40
(Sequence ID No. 196)
5′-ATGGTTCGTCTGCCTCTGCAGTGCGTCCTCTGGGGCTGCTTGCTGA
CCGCTGTCCATCCAGAACCACCCACTGCATGCAGAGAAAAACAGTACCT
AATAAACAGTCAGTGCTGTTCTTTGTGCCAGCCAGGACAGAAACTGGTG
AGTGACTGCACAGAGTTCACTGAAACGGAATGCCTTCCTTGCGGTGAAA
GCGAATTCCTAGACACCTGGAACAGAGAGACACACTGCCACCAGCACAA
ATACTGCGACCCCAACCTAGGGCTTCGGGTCCAGCAGAAGGGCACCTCA
GAAACAGACACCATCTGCACCTGTGAAGAAGGCTGGCACTGTACGAGTG
AGGCCTGTGAGAGCTGTGTCCTGCACCGCTCATGCTCGCCCGGCTTTGG
GGTCAAGCAGATTGCTACAGGGGTTTCTGATACCATCTGCGAGCCCTGC
CCAGTCGGCTTCTTCTCCAATGTGTCATCTGCTTTCGAAAAATGTCACC
CTTGGACAAGCTGTGAGACCAAAGACCTGGTTGTGCAACAGGCAGGCAC
AAACAAGACTGATGTTGTCTGTGGTCCCCAGGATCGGCTGAGAGCCCTG
GTGGTGATCCCCATCATCTTCGGGATCCTGTTTGCCATCCTCTTGGTGC
TGGTCTTTATCAAAAAGGTGGCCAAGAAGCCAACCAATAAGGCCCCCCA
CCCCAAGCAGGAACCCCAGGAGATCAATTTTCCCGACGATCTTCCTGGC
TCCAACACTGCTGCTCCAGTGCAGGAGACTTTACATGGATGCCAACCGG
TCACCCAGGAGGATGGCAAAGAGAGTCGCATCTCAGTGCAGGAGAGACA
G-3′
Nucleic Acid Coding for 4-1BBL
(Sequence ID No. 197)
5′-ATGGAATACGCCTCTGACGCTTCACTGGACCCCGAAGCCCCGTGGC
CTCCCGCGCCCCGCGCTCGCGCCTGCCGCGTACTGCCTTGGGCCCTGGT
CGCGGGGCTGCTGCTGCTGCTGCTGCTCGCTGCCGCCTGCGCCGTCTTC
CTCGCCTGCCCCTGGGCCGTGTCCGGGGCTCGCGCCTCGCCCGGCTCCG
CGGCCAGCCCGAGACTCCGCGAGGGTCCCGAGCTTTCGCCCGACGATCC
CGCCGGCCTCTTGGACCTGCGGCAGGGCATGTTTGCGCAGCTGGTGGCC
CAAAATGTTCTGCTGATCGATGGGCCCCTGAGCTGGTACAGTGACCCAG
GCCTGGCAGGCGTGTCCCTGACGGGGGGCCTGAGCTACAAAGAGGACAC
GAAGGAGCTGGTGGTGGCCAAGGCTGGAGTCTACTATGTCTTCTTTCAA
CTAGAGCTGCGGCGCGTGGTGGCCGGCGAGGGCTCAGGCTCCGTTTCAC
TTGCGCTGCACCTGCAGCCACTGCGCTCTGCTGCTGGGGCCGCCGCCCT
GGCTTTGACCGTGGACCTGCCACCCGCCTCCTCCGAGGCTCGGAACTCG
GCCTTCGGTTTCCAGGGCCGCTTGCTGCACCTGAGTGCCGGCCAGCGCC
TGGGCGTCCATCTTCACACTGAGGCCAGGGCACGCCATGCCTGGCAGCT
TACCCAGGGCGCCACAGTCTTGGGACTCTTCCGGGTGACCCCCGAAATC
CCAGCCGGACTCCCTTCACCGAGGTCGGAA-3′

### (5) First Screening Method (First Implementation)

Screening was performed using the first candidate CAR-Ts described in (3) above and the APCs prepared in (4) above. Specifically, the first candidate CAR-Ts and the APCs were seeded in each well (24-well plate) such that the number of the first candidate CAR-Ts was 2×10⁶ and the number of the APCs was 1×10⁵, and then they were cultured together for 7 days. Before use, the APCs were treated with 20 Gy of γ rays and were then pulsed with an NY-ESO-1₁₅₇₋₁₆₅ peptide (NY-ESO-1₁₅₇ peptide, SLLMWITQC: Sequence ID No. 206) at a concentration of 1 to 10 μg/mL, and peptides that did not bind to the HLA-A*02:01 were removed therefrom after the pulsing process. An RPMI1640 culture medium containing gentamicin at a concentration of 50 μg/mL, human AB serum (manufactured by Sigma) at a concentration of 10%, human IL-2 (manufactured by Roche) at a concentration of 10 IU/mL, and human IL-15 (manufactured by PeproTech) at a concentration of 10 ng/mL was used as the culture medium used for the above-mentioned coculture of the first candidate CAR-Ts and the APCs. After the coculture, the first candidate CAR-Ts were collected, and coculture of the first candidate CAR-Ts and the APCs was performed two more times (three times in total) in the same conditions. Thus, the first candidate CAR-Ts expressing the hH-3M4E4L CARs capable of binding to the A2/NY-ESO-1₁₅₇ was enriched.

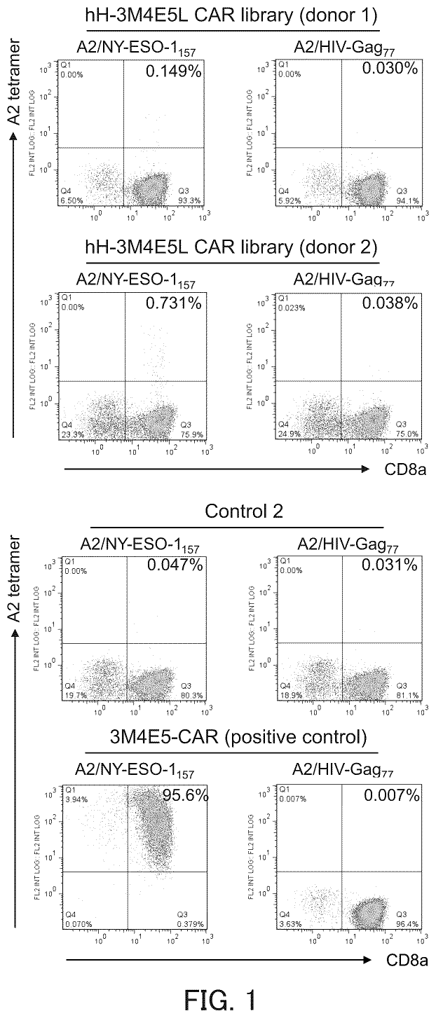

Next, after the enrichment of the first candidate CAR-Ts, the first candidate CAR-Ts were collected and stained with a PE-labeled A2/NY-ESO-1₁₅₇ tetramer at a concentration of 20 μg/mL. The PE-labeled A2/NY-ESO-1₁₅₇ tetramer was prepared by mixing a biotinylated HLA-A2/NY-ESO-1₁₅₇ monomer and PE-labeled streptavidin (manufactured by Thermo Fisher Scientific). After the above-mentioned staining, the T cells were further stained with a PC5-labeled anti-human CD8α mAb (clone: B9.11), an FITC-labeled anti-human CD4 mAb (clone: OKT4), and a V450-labeled anti-human NGFR mAb (clone: C40-1457). The concentrations of the antibodies were about 10 μg/mL. Then, regarding the first candidate CAR-Ts that had been subjected to tetramer staining, it was confirmed using the flow cytometer if first candidate CAR-Ts expressing CARs capable of binding to the A2/NY-ESO-1₁₅₇ were present (donor 1). The same test was performed using T cells derived from another healthy subject (donor 2). A positive control was performed in the same manner, except that an expression vector that included a nucleic acid coding for a tagged 3M4E5HL CAR was used instead of the expression vectors for expressing the hH-3M4E4L CAR library. A control 1 was performed in the same manner, except that a tetramer consisting of a complex (A2/HIV-Gag₇₇) of HLA-A*02:01 and an HIV Gag₇₇₋₈₅ peptide (SLYNTVATL: Sequence ID No. 198) was used instead of the A2/NY-ESO-1₁₅₇ tetramer. A control 2 was performed in the same manner, except that the expression vectors for expressing the hH-3M4E4L CAR library were not introduced. FIG. 1 shows the results obtained when gating of FSC⁺-SSC⁺-NGFR⁺ cells was performed.

FIG. 1 shows dot plots showing the results of flow cytometry. In FIG. 1, the horizontal axes indicate the average fluorescence intensity of CD8a, and the vertical axes indicate the average fluorescence intensity of the A2 tetramer. As shown in FIG. 1, the T cells (control 2) into which the expression vectors for expressing the hH-3M4E4L CAR library had not been introduced did not include T cells capable of binding to the A2/NY-ESO-1₁₅₇ or A2/HIV-Gag₇₇. Moreover, it could be confirmed that the CAR-Ts (3M4E5-CAR, positive control) expressing the 3M4E5HL CAR were capable of binding to the A2/NY-ESO-1₁₅₇ but did not bind to the A2/HIV-Gag₇₇ because a CD8⁺-A2 tetramer⁺ fraction (the upper right fraction in each dot plot) was present when A2/NY-ESO-1₁₅₇ tetramer staining was performed, and the CD8⁺-A2 tetramer fraction was not present when A2/HIV-Gag₇₇ tetramer staining was performed. Furthermore, in the case of the first candidate CAR-Ts (donors 1 and 2), the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed. It was found from these results that the first candidate CAR-Ts included first candidate CAR-Ts expressing CARs capable of binding to the A2/NY-ESO-1₁₅₇.

Next, first candidate CAR-Ts expressing CARs capable of binding to the A2/NY-ESO-1₁₅₇ were selected, and the CARs expressed by the first candidate CAR-Ts were identified. Specifically, the first candidate CAR-Ts were stained in the same manner as in (5) above. Then, cell sorting was performed on the first candidate CAR-Ts using a flow cytometer (FACSAria (registered trademark), manufactured by Becton Dickinson), and thus an NGFR⁺-CD8⁺-A2/NY-ESO-1₁₅₇ tetramer⁺ fraction or NGFR⁺-CD4⁺-A2/NY-ESO-1₁₅₇ tetramer⁺ fraction was collected as first candidate CAR-Ts expressing CARs capable of binding to the A2/NY-ESO-1₁₅₇. The total RNA was extracted from the obtained first candidate CAR-Ts using a total RNA extraction reagent (TRIzol (registered trademark), manufactured by Ambion). cDNA was synthesized from the total RNA using a reverse transcriptase (Superscript (registered trademark) III, manufactured by Thermo Fisher Scientific).

PCR in which the obtained cDNA and a mixture of a forward primer 1 below and a reverse primer 1 below were used was performed to amplify nucleic acids coding for scFvs. A cloning kit (Gibson Assembly Master Mix, manufactured by New England Biolab) was used to introduce the obtained nucleic acids coding for scFvs into pMX/CAR expression vectors (pMX/scFv expression vectors) such that the nucleic acid coding for an scFv bound to the 5′ end of the partial region of human CD28. The pMX/CAR expression vectors were prepared by linking a nucleic acid (Sequence ID No. 169) coding for the partial region, the transmembrane domain, and the intracellular signaling domain of human CD28 and a nucleic acid (Sequence ID No. 171) coding for the intracellular signaling domain of human CD32 such that these nucleic acids were arranged in the stated order from the 5′ end side and introducing the thus obtained product into the pMX expression vector. Then, sequencing of the obtained pMX/scFv expression vectors was performed using the Sanger's method, and thus base sequences coding for CARs (a polynucleotide (Sequence ID No. 138) coding for an H1-3M4E5L CAR and a polynucleotide (Sequence ID No. 157) coding for an H73-3M4E5L CAR) were identified. Moreover, the amino acid sequences of the scFvs (H1-3M4E5L (Sequence ID No. 98) and H73-3M4E5L (Sequence ID No. 117)) were identified based on the above-mentioned base sequences. Then, the CDRH1, CDRH2, and the CDRH3 in the new heavy-chain variable region (HA) and those in the new heavy-chain variable region (HC), which are shown in Table 1A above, were identified based on the amino acid sequences of the scFvs. Furthermore, the pMX/scFv expression vectors were used to prepare a pMX/H1-3M4E5L expression vector that included a polynucleotide coding for the H1-3M4E5L and a pMX/H73-3M4E5L expression vector that included a polynucleotide coding for the H73-3M4E5L.

Forward Primer 1
(Sequence ID No. 199)
5′-ATCCCAGTGTGGTGGTACGGG-3′
Reverse Primer 1
(Sequence ID No. 200)
5′-GGGTACATCACTTCGATTGC-3′

(6) Analysis of First Candidate scFvs

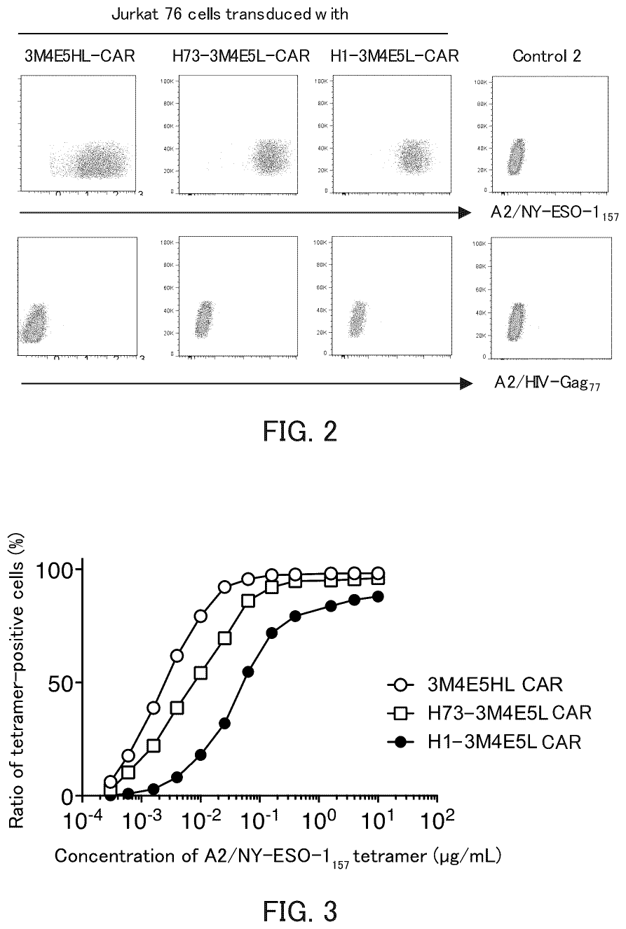

A culture supernatant containing GaLV-pseudotyped retroviruses was prepared in the same manner as in (3) above, except that the pMX/H1-3M4E5L expression vector or the pMX/H73-3M4E5L expression vector was used instead of the expression vectors for expressing the hH-3M4E4L CAR library. Next, the pMX/H1-3M4E5L expression vector or the pMX/H73-3M4E5L expression vector was introduced into Jurkat 76 cells by adding the above-mentioned culture supernatant containing GaLV-pseudotyped retroviruses, and a CAR (H1-3M4E5L CAR or H73-3M4E5L CAR) that included the H1-3M4E5L or H73-3M4E5L as an scFv was expressed. The Jurkat 76 cells that had expressed the CARs were reacted with the PE-labeled anti-human NGFR mAb. After the above-mentioned reaction, Jurkat 76 cells (H1-3M4E5L CAR-Ts or H73-3M4E5L CAR Ts) expressing the H1-3M4E5L CAR and the H73-3M4E5L CAR were purified using the anti-PE microbeads. The H1-3M4E5L CAR-Ts and the H73-3M4E5L CAR Ts were stained with the PE-labeled A2/NY-ESO-1₁₅₇ tetramer at a concentration of 5 μg/mL, and were then stained with the V450-labeled anti-human NGFR mAb. The stained H1-3M4E5L CAR-Ts and H73-3M4E5L CAR Ts were analyzed by flow cytometry. A positive control was performed in the same manner, except that an expression vector that included a nucleic acid coding for a tagged 3M4E5LH CAR was used instead of the pMX/H1-3M4E5L expression vector or the pMX/H73-3M4E5L expression vector. A control 1 was performed in the same manner, except that the A2/HIV-Gag₇₇ tetramer was used instead of the A2/NY-ESO-1₁₅₇ tetramer. A control 2 was performed in the same manner, except that the pMX/H1-3M4E5L expression vector or pMX/H73-3M4E5L expression vector was not introduced. FIG. 2 shows the results obtained when gating of FSC⁺-SSC⁺-NGFR⁺ cells was performed.

FIG. 2 shows dot plots showing the results of flow cytometry. In FIG. 2, the horizontal axes indicate the average fluorescence intensity resulting from A2/NY-ESO-1₁₅₇ tetramer staining or A2/HIV-Gag₇₇ tetramer staining, and the vertical axes indicate the average side scatter (SSC) intensity. As shown in FIG. 2, the Jurkat 76 cells (control 2) into which the pMX/H1-3M4E5L expression vector or pMX/H73-3M4E5L expression vector had not been introduced did not include cells capable of binding to the A2/NY-ESO-1₁₅₇ or A2/HIV-Gag₇₇. Moreover, it could be confirmed that the 3M4E5HL CAR-Ts (3M4E5 CAR, positive control) were capable of binding to the A2/NY-ESO-1₁₅₇ but did not bind to the A2/HIV-Gag₇₇ because substantially all the cells were present as A2 tetramer⁺ cells when A2/NY-ESO-1₁₅₇ tetramer staining was performed, and substantially all the cells were present as A2 tetramer cells when A2/HIV-Gag₇₇ tetramer staining was performed. Furthermore, in the case of the H1-3M4E5L CAR-Ts and the H73-3M4E5L CAR-Ts, substantially all the cells were present as A2 tetramer⁺ cells when A2/NY-ESO-1₁₅₇ tetramer staining was performed, and substantially all the cells were present as A2 tetramer cells when A2/HIV-Gag₇₇ tetramer staining was performed. It was found from these results that the H1-3M4E5L CAR-Ts and the H73-3M4E5L CAR-Ts were capable of binding to the A2/NY-ESO-1₁₅₇ but did not bind to the A2/HIV-Gag₇₇. That is, it was found that the first screening method of the present invention could be used to screen scFvs capable of binding to a target antigen.

(7) Avidity of First Candidate scFvs

Next, flow cytometry analysis was performed on the H1-3M4E5L CAR-Ts and the H73-3M4E5L CAR Ts, which had been obtained in (6) above, and Jurkat 76 cells expressing the 3M4E5LH CAR (3M4E5LH CAR-Ts) in the same manner as in (6) above, except that they were stained with the PE-labeled A2/NY-ESO-1₁₅₇ tetramer at a predetermined concentration (0.0004194304, 0.001048576, 0.00262144, 0.0065536, 0.016384, 0.04096, 0.1024, 0.256, 0.64, 1.6, 4, or 10 μg/mL), and were then stained with the V450-labeled anti-human NGFR mAb. Then, the ratio of A2/NY-ESO-1₁₅₇ tetramer-positive cells was calculated for each A2/NY-ESO-1₁₅₇ tetramer concentration. FIG. 3 shows the results.

FIG. 3 is a graph indicating the ratios of A2/NY-ESO-1₁₅₇ tetramer-positive cells. In FIG. 3, the horizontal axis indicates the concentration of the A2/NY-ESO-1₁₅₇ tetramer, and the vertical axis indicates the ratio of A2/NY-ESO-1₁₅₇ tetramer-positive cells. As shown in FIG. 3, the ratios of the A2/NY-ESO-1₁₅₇ tetramer-positive cells in the CAR-Ts increased in a manner dependent on the concentration of the A2/NY-ESO-1₁₅₇ tetramer. It was necessary to increase the concentration of the A2/NY-ESO-1₁₅₇ tetramer in order to obtain, from the H1-3M4E5L CAR-Ts and the H73-3M4E5L CAR Ts, the A2/NY-ESO-1₁₅₇ tetramer-positive cells at the same level as those from the 3M4E5LH CAR-Ts, and it was thus found that the scFvs in these CAR-Ts were different in avidity for the A2/NY-ESO-1₁₅₇ tetramer. That is, it was found that the first screening method of the present invention could be used to screen scFvs different in binding avidity for a target antigen.

(8) Functions of First Candidate scFvs

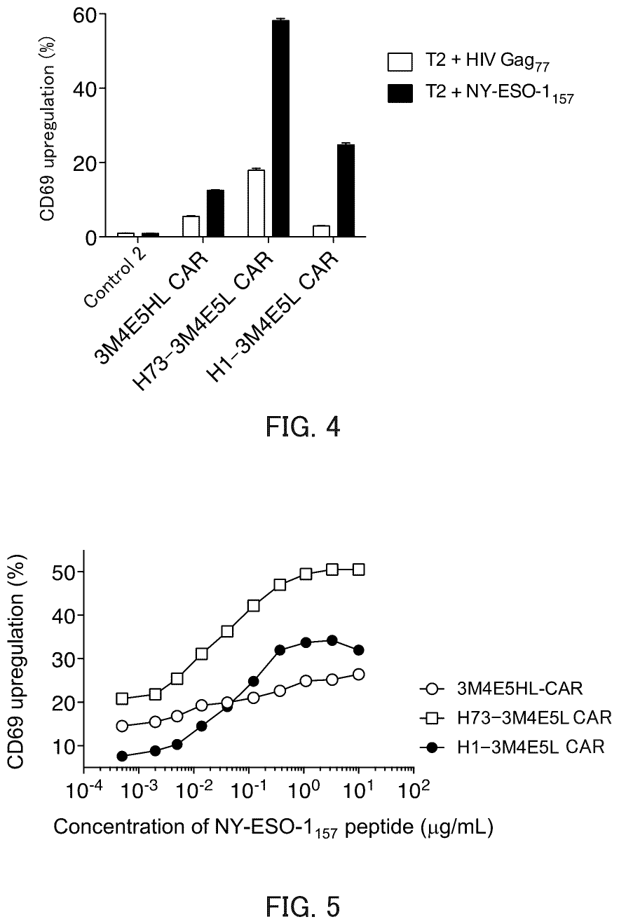

Next, it was examined if the H1-3M4E5L CAR-Ts and the H73-3M4E5L CAR-Ts bound to the target antigen and were thus activated. Specifically, the H1-3M4E5L CAR-Ts or H73-3M4E5L CAR-Ts and the T2 cells were seeded in each well (96-well plate) such that the number of the H1-3M4E5L CAR-Ts or H73-3M4E5L CAR-Ts was 3×10⁵ and the number of the T2 cells was 5×10⁴, and then they were cultured together for 5 hours. Before use, the T2 cells (HLA-A*02:01-expressing cells) were pulsed with the NY-ESO-1₁₅₇ peptide at a predetermined concentration (0.0005, 0.0015, 0.004, 0.01, 0.04, 0.122, 0.366, 1.11, 3.33, or 10 μg/mL) (T2+NY-ESO-1₁₅₇). An RPMI1640 culture medium containing FCS at a concentration of 10% was used as the culture solution for the coculture. After the above-mentioned coculture, the H1-3M4E5L CAR-Ts and the H73-3M4E5L CAR-Ts were collected, and were then stained with an FITC-labeled anti-human CD69 mAb (clone: FN50) and a V450-labeled anti-human NGFR mAb. The average fluorescence intensity of CD69 in NGFR⁺ cells from the stained H1-3M4E5L CAR-Ts and H73-3M4E5L CAR-Ts was measured using the flow cytometer. A positive control was performed in the same manner, except that the 3M4E5HL CAR-Ts obtained in (6) above were used. A control 1 was performed in the same manner, except that T2 cells (T2+HIV Gag₇₇) pulsed with the HIV Gag₇₇₋₈₅ peptide instead of the NY-ESO-1₁₅₇ peptide were used. A control 2 was performed in the same manner, except that the pMX/H1-3M4E5L expression vector was not introduced. Then, the rates of an increase in CD69 expression in the samples were calculated for each peptide concentration using the average fluorescence intensity of CD69 in the control 2 as a standard. FIGS. 4 and 5 show the results.

FIG. 4 is a graph indicating changes in a CD69 expression level obtained when the peptide concentration was 10 μg/mL. In FIG. 4, the horizontal axis indicates the types of samples, and the vertical axis indicates the rate of an increase in CD69 expression. As shown in FIG. 4, the CD69 expression did not increase in the control 2. On the other hand, when the 3M4E5HL CAR-Ts (positive control) were stimulated with the T2+NY-ESO-1₁₅₇, it could be confirmed that the CD69 expression increased compared with the case of the stimulation with the T2+HIV Gag₇₇, and the CAR-Ts bound to the A2/NY-ESO-1₁₅₇ and were activated. When the H1-3M4E5L CAR-Ts and the H73-3M4E5L CAR-Ts were stimulated with the T2+NY-ESO-1₁₅₇, the CD69 expression increased compared with the case of the stimulation with the T2+HIV Gag₇₇, and the CAR-Ts bound to the A2/NY-ESO-1₁₅₇ and were activated. Furthermore, in the H1-3M4E5L CAR-Ts and the H73-3M4E5L CAR-Ts, the rates of an increase in CD69 expression calculated based on the rates of an increase in CD69 expression level obtained when the stimulation with the T2+HIV Gag₇₇ was performed were higher than that in the 3M4E5 HL CAR-Ts. It was thus found that the H1-3M4E5L CAR-Ts and the H73-3M4E5L CAR-Ts were more specific to the A2/NY-ESO-1₁₅₇ than the 3M4E5 HL CAR-Ts. Moreover, it was found that the first screening method of the present invention could be used to screen scFvs that were functional in CAR-T cells and had a higher specificity.

Next, the results of changes in CD69 expression in the case where the NY-ESO-1₁₅₇ peptide was serially diluted are shown in FIG. 5. FIG. 5 is a graph indicating changes in a CD69 expression level. In FIG. 5, the horizontal axis indicates the NY-ESO-1₁₅₇ peptide concentration, and the vertical axis indicates the rate of an increase in CD69 expression. As shown in FIG. 5, in the CAR-Ts, the CD69 expression increased in a manner dependent on the NY-ESO-1₁₅₇ peptide concentration. It was found that, in the H1-3M4E5L CAR-Ts and the H73-3M4E5L CAR Ts, the CD 69 expression increased more sharply and the specificity for the A2/NY-ESO-1₁₅₇ was higher compared with the 3M4E5LH CAR-Ts. That is, it was found that the first screening method of the present invention could be used to screen scFvs that were functional in CAR-T cells and had a higher specificity.

### (9) Preparation of Second CAR Library

A second CAR library was prepared using the heavy-chain variable region (HA) screened from the first CAR library. Specifically, the nucleic acid coding for the heavy-chain variable region (3M4E5H) in the pMX expression vector into which the nucleic acid coding for the tagged 3M4E5LH CAR had been introduced was substituted with a nucleic acid coding for the heavy-chain variable region (HA) (3M4E5L-H1 expression vector). Next, the nucleic acid coding for the light-chain variable region (3M4E5L) in the 3M4E5L-H1 expression vectors was substituted with a library that included nucleic acids coding for light-chain variable regions described in (2) above. Thus, the expression vectors that included the second CAR library (expression vectors for expressing the hK-H1 CAR library) were prepared. It should be noted that, regarding the heterogeneity of the heavy-chain variable regions in the hK-H1 CAR library, it was confirmed through restriction enzyme mapping and sequencing of the regions using the Sanger's method that the number of types of heavy-chain variable regions was about 1×10⁶.

### (10) Preparation of Second CAR Library-Expressing T Cells (Second Candidate CAR-Ts)

The hK-H1 CAR library was introduced into T cells in the same manner as in (3) above, except that the expression vectors for expressing the hK-H1 CAR library were used instead of the expression vectors for expressing the hH-3M4E4L CAR library. Then, the resulting T cells were purified and used as second candidate CAR-Ts.

### (11) First Screening Method (Second Implementation)

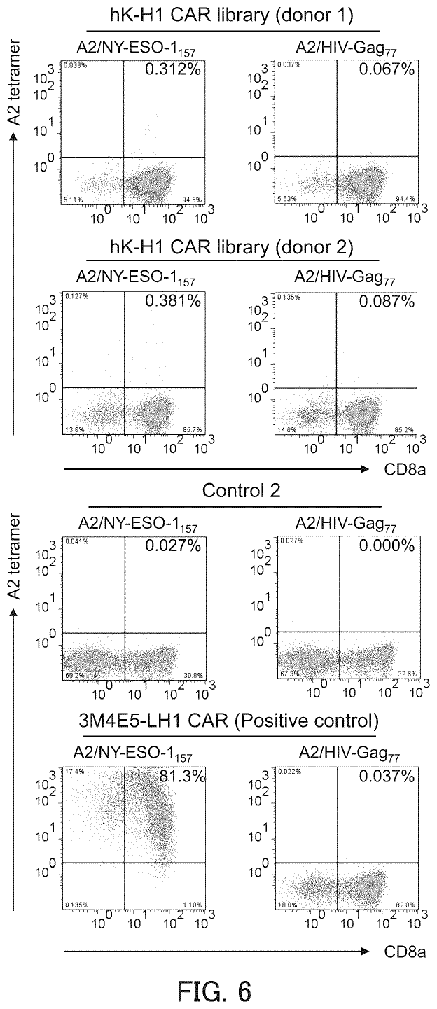

Next, in the same manner as in (5) above, except that the second candidate CAR-Ts were used instead of the first candidate CAR-Ts, it was confirmed if second candidate CAR-Ts expressing CARs capable of binding to the A2/NY-ESO-1₁₅₇ were present (donor 1). The same test was performed using a library that included nucleic acids of heavy-chain variable regions derived from human peripheral blood B cells obtained from another healthy subject (donor 2). A positive control was performed in the same manner, except that a pMX/3M4E5L-H1 expression vector was used instead of the expression vectors for expressing the hK-H1 CAR library. A control 1 was performed in the same manner, except that the A2/HIV-Gag₇₇ tetramer was used instead of the A2/NY-ESO-1₁₅₇ tetramer. A control 2 was performed in the same manner, except that the expression vectors for expressing the hH-3M4E5L CAR library were not introduced. FIG. 6 shows the results obtained when gating of FSC⁺-SSC⁺-NGFR⁺ cells was performed.

FIG. 6 shows dot plots showing the results of flow cytometry. In FIG. 6, the horizontal axes indicate the average fluorescence intensity of CD8a, and the vertical axes indicate the average fluorescence intensity of the A2 tetramer. As shown in FIG. 6, the T cells (control 2) into which the expression vectors for expressing the hK-H1 CAR library had not been introduced did not include T cells capable of binding to the A2/NY-ESO-1₁₅₇ or A2/HIV-Gag₇₇. Moreover, it could be confirmed again that the CAR-Ts (3M4E5L-H1 CAR, positive control) expressing the 3M4E5L-H1 CAR were capable of binding to the A2/NY-ESO-1₁₅₇ but did not bind to the A2/HIV-Gag₇₇ because a CD8⁺-A2 tetramer⁺ fraction (the upper right fraction in each dot plot) was present when A2/NY-ESO-1₁₅₇ tetramer staining was performed, and the CD8⁺-A2 tetramer⁺ fraction was not present when A2/HIV-Gag₇₇ tetramer staining was performed. Furthermore, in the case of the second candidate CAR-Ts (donors 1 and 2), the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed. It was found from these results that the second candidate CAR-Ts included second candidate CAR-Ts expressing CARs capable of binding to the A2/NY-ESO-1₁₅₇.

Next, second candidate CAR-Ts expressing CARs capable of binding to the A2/NY-ESO-1₁₅₇ were selected, and the CARs expressed by the second candidate CAR-Ts were identified. Specifically, the second candidate CAR-Ts were stained with the A2/NY-ESO-1₁₅₇ tetramer. Then, cell sorting was performed on the second candidate CAR-Ts using a flow cytometer, and thus an NGFR⁺-CD8⁺-A2/NY-ESO-1₁₅₇ tetramer⁺ fraction or NGFR⁺-CD4⁺-A2/NY-ESO-1₁₅₇-tetramer⁺ fraction was collected as second candidate CAR-Ts expressing CARs capable of binding to the A2/NY-ESO-1₁₅₇. Then, scFvs were prepared in the same manner as in (5) above, except that second candidate CAR-Ts expressing CARs capable of binding to the A2/NY-ESO-1₁₅₇ were used instead of the first candidate CAR-Ts expressing CARs capable of binding to the A2/NY-ESO-1₁₅₇, and the resulting scFvs were introduced into pMX/CAR expression vectors. Then, sequencing of the obtained pMX/CAR expression vectors into which the scFvs had been introduced was performed using the Sanger's method, and thus base sequences coding for CARs (a polynucleotide coding for K52-H1 CAR (Sequence ID No. 139), a polynucleotide coding for K73-H1 CAR (Sequence ID No. 140), a polynucleotide coding for K121-H1 CAR (Sequence ID No. 141), a polynucleotide coding for K124-H1 CAR (Sequence ID No. 142), a polynucleotide coding for K125-H1 CAR (Sequence ID No. 143), a polynucleotide coding for K131-H1 CAR (Sequence ID No. 144), a polynucleotide coding for K145-H1 CAR (Sequence ID No. 145), a polynucleotide coding for K151-H1 CAR (Sequence ID No. 146), a polynucleotide coding for K160-H1 CAR (Sequence ID No. 147), and a polynucleotide coding for K173-H1 CAR (Sequence ID No. 148)) were identified. Moreover, the amino acid sequences of the scFvs (K52-H1 (Sequence ID No. 99), K73-H1 (Sequence ID No. 100), K121-H1 (Sequence ID No. 101), K124-H1 (Sequence ID No. 102), K125-H1 (Sequence ID No. 103), K131-H1 (Sequence ID No. 104), K145-H1 (Sequence ID No. 105), K151-H1 (Sequence ID No. 106), K160-H1 (Sequence ID No. 107), and K173-H1 (Sequence ID No. 108)) were identified based on the above-mentioned base sequences. Then, the CDRH1, CDRH2, and the CDRH3 in the new light-chain variable regions (LB) to (LJ), which are shown in Table 1B above, were identified based on the amino acid sequences of the scFvs.

(12) Analysis of Second Candidate scFvs

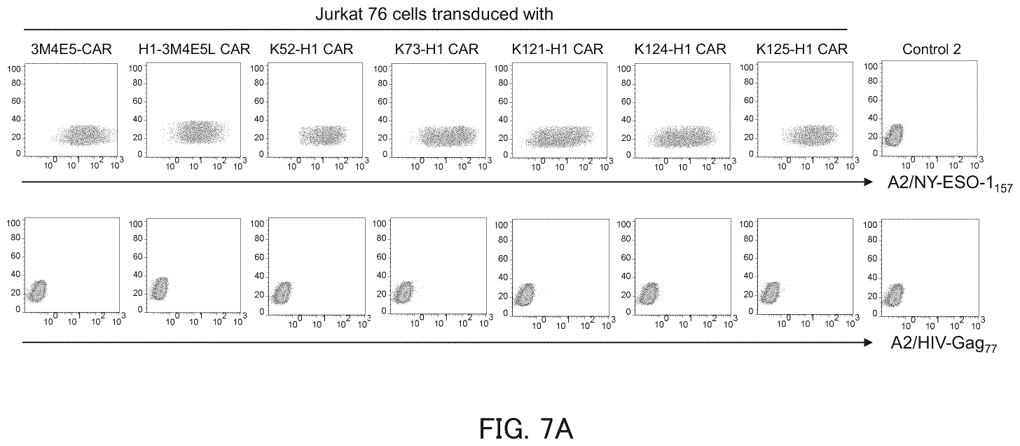

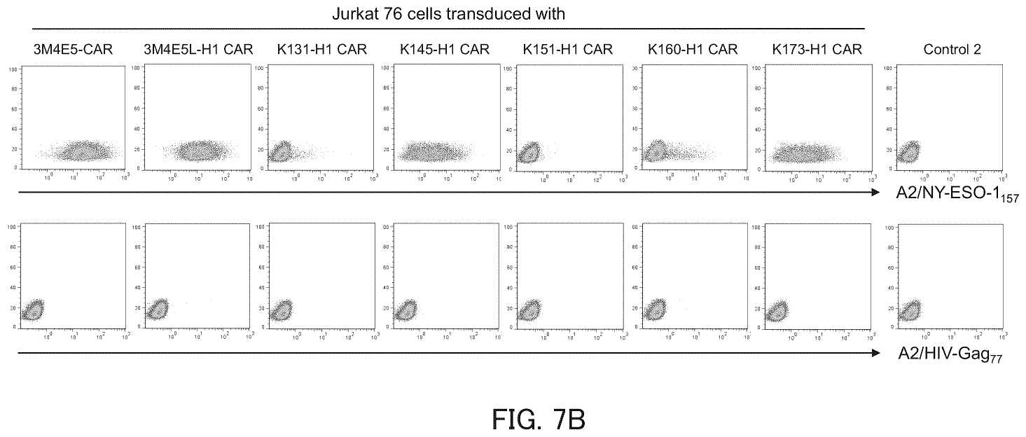

A culture supernatant containing GaLV-pseudotyped retroviruses was prepared in the same manner as in (3) above, except that the pMX/CAR expression vectors into which polynucleotides coding for the scFvs described in (11) above had been introduced instead of the polynucleotides coding for the scFvs in the expression vectors for expressing the hH-3M4E4L CAR library were used. Next, the pMX/CAR expression vectors into which the polynucleotides coding for the scFvs had been introduced were introduced into Jurkat 76 cells by adding the above-mentioned culture supernatant containing GaLV-pseudotyped retroviruses, and a CAR that included the K52-H1, K73-H1, K121-H1, K124-H1, K125-H1, K131-H1, K145-H1, K151-H1, K160-H1, or K173-H1 as an scFv was expressed. The Jurkat 76 cells that had expressed the CARs were reacted with the PE-labeled anti-human NGFR mAb. After the above-mentioned reaction, Jurkat 76 cells expressing the CARs were purified using the anti-PE microbeads. The purified Jurkat 76 cells expressing the CARs were stained with the PE-labeled A2/NY-ESO-1₁₅₇ tetramer at a concentration of 5 μg/mL, and were then stained with the V450-labeled anti-human NGFR mAb. The stained Jurkat 76 cells expressing the CARs were analyzed by flow cytometry. A process was performed in the same manner, except that the pMX/H1-3M4E5L expression vector was used instead of the pMX/CAR expression vectors into which the scFvs had been introduced. A positive control was performed in the same manner, except that an expression vector that included the nucleic acid coding for the tagged 3M4E5LH CAR was used instead of the pMX/CAR expression vectors into which the scFvs had been introduced. A control 1 was performed in the same manner, except that the A2/HIV-Gag₇₇ tetramer was used instead of the A2/NY-ESO-1₁₅₇ tetramer. A control 2 was performed in the same manner, except that the pMX/CAR expression vectors into which the scFvs had been introduced were not introduced. FIGS. 7(A) and 7(B) show the results obtained when gating of NGFR⁺ cells was performed.

FIGS. 7(A) and 7(B) show dot plots showing the results of flow cytometry. In FIGS. 7(A) and 7(B), the horizontal axes indicate the average fluorescence intensity of the A2 tetramer, and the vertical axes indicate the average side scatter (SSC) intensity. As shown in FIGS. 7(A) and 7(B), the Jurkat 76 cells (control 2) into which the scFv-introduced pMX/CAR expression vectors had not been introduced did not include cells capable of binding to the A2/NY-ESO-1₁₅₇ or A2/HIV-Gag₇₇. Moreover, it could be confirmed that the 3M4E5LH CAR-Ts (3M4E5-CAR positive control) were capable of binding to the A2/NY-ESO-1₁₅₇ but did not bind to the A2/HIV-Gag₇₇ because substantially all the cells were present in the A2 tetramer⁺ fraction when A2/NY-ESO-1₁₅₇ tetramer staining was performed, and the A2 tetramer⁺ fraction was not present when A2/HIV-Gag₇₇ tetramer staining was performed. Substantially all or some T cells expressing CARs that included H1-3M4E5L, K52-H1, K73-H1, K121-H1, K124-H1, K125-H1, K131-H1, K145-H1, K151-H1, K160-H1, or K173-H1 were present in the A2 tetramer⁺ fraction when A2/NY-ESO-1₁₅₇ tetramer staining was performed, and the A2 tetramer⁺ fraction was not present when A2/HIV-Gag₇₇ tetramer staining was performed. It was found from these results that the CARs that included H1-3M4E5L, K52-H1, K73-H1, K121-H1, K124-H1, K125-H1, K131-H1, K145-H1, K151-H1, K160-H1, or K173-H1 were capable of binding to the A2/NY-ESO-1₁₅₇ but did not bind to the A2/HIV-Gag₇₇. That is, it was found that the first screening method of the present invention could be used to screen scFvs that included new heavy-chain variable regions and new light-chain variable regions capable of binding to a target antigen. Moreover, since CAR-expressing T cells that were stained with the A2/NY-ESO-1₁₅₇ tetramer to different staining degrees were present, it was found that scFvs that included heavy-chain variable regions and light-chain variable regions having different avidity for a target antigen could be screened.

### Example 2

It was confirmed that the screening method of the present invention in which the CAR library of the present invention is used could be used to screen scFvs capable of binding to A2/NY-ESO-1₁₅₇.

### (1) Preparation of First CAR Library

An hL-3M4E4H scFv library was prepared by substituting the nucleic acid coding for the light-chain variable region on the 5′ side of the nucleic acid coding for the 3M4E5LH scFv described in (1) of Example 1 above with the above-mentioned library that included the nucleic acids of the light-chain variable regions derived from human peripheral blood B cells. Then, a tagged hL-3M4E4H CAR library was prepared in the same manner as in (1) of Example 1 above, except that the hL-3M4E4H scFv library was used instead of the nucleic acid coding for the 3M4E5LH scFv. The tagged hL-3M4E4H CAR library was introduced into pMX expression vectors to prepare expression vectors for expressing the hL-3M4E4H CAR library. It should be noted that the heterogeneity of the heavy-chain variable regions of the hL-3M4E4H CAR library was confirmed through restriction enzyme mapping and sequencing of the regions using the Sanger's method.

### (2) Preparation of First CAR Library-Expressing T Cells (First Candidate CAR-Ts)

The hL-3M4E4H CAR library was introduced into T cells in the same manner as in (3) of Example 1 above, except that the expression vectors for expressing the hL-3M4E4H CAR library were used instead of the expression vectors for expressing the hH-3M4E4L CAR library. Then, the resulting T cells were purified and used as first candidate CAR-Ts.

### (3) First Screening Method

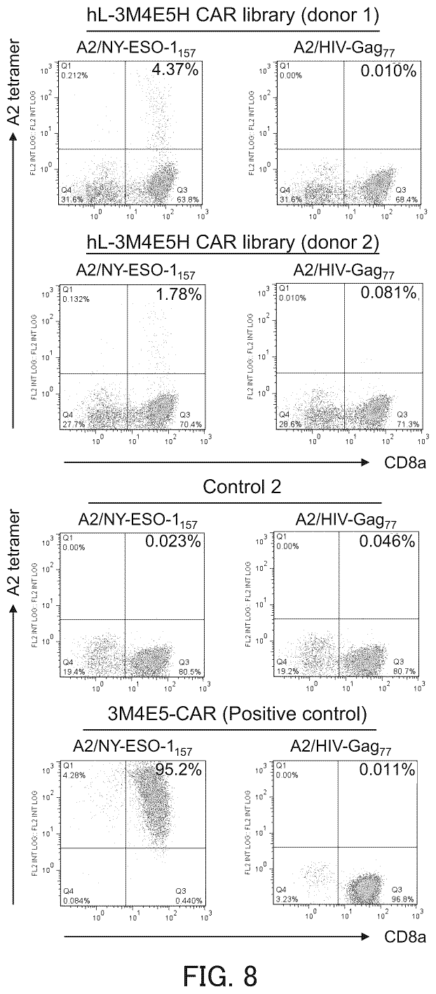

In the same manner as in (5) of Example 1 above, except that the first candidate CAR-Ts described in (2) of Example 2 were used instead of the first candidate CAR-Ts described in (3) of Example 1 above, it was confirmed that first candidate CAR-Ts expressing CARs capable of binding to the A2/NY-ESO-1₁₅₇ were present (donor 1). The same test was performed using a library that included nucleic acids of heavy-chain variable regions derived from human peripheral blood B cells obtained from another healthy subject (donor 2). A positive control was performed in the same manner, except that an expression vector that included a nucleic acid coding for the tagged 3M4E5LH CAR was used instead of the expression vectors for expressing the hH-3M4E4L CAR library. A control 1 was performed in the same manner, except that the A2/HIV-Gag₇₇ tetramer was used instead of the A2/NY-ESO-1₁₅₇ tetramer. A control 2 was performed in the same manner, except that the expression vectors for expressing the hL-3M4E4H CAR library were not introduced. FIG. 8 shows the results obtained when gating of FSC⁺-SSC⁺-NGFR⁺ cells was performed.

FIG. 8 shows dot plots showing the results of flow cytometry. In FIG. 8, the horizontal axes indicate the average fluorescence intensity of CD8a, and the vertical axes indicate the average fluorescence intensity of the A2 tetramer. As shown in FIG. 8, the T cells (control 2) into which the expression vectors for expressing the hL-3M4E4H CAR library had not been introduced did not include T cells capable of binding to the A2/NY-ESO-1₁₅₇ or A2/HIV-Gag₇₇. Moreover, it could be confirmed that the CAR-Ts (3M4E5-CAR, positive control) expressing the 3M4E5LH CAR were capable of binding to the A2/NY-ESO-1₁₅₇ but did not bind to the A2/HIV-Gag₇₇ because a CD8⁺-A2 tetramer⁺ fraction (the upper right fraction in each dot plot) was present when A2/NY-ESO-1₁₅₇ tetramer staining was performed, and the CD8⁺-A2 tetramert fraction was not present when A2/HIV-Gag₇₇ tetramer staining was performed. Furthermore, in the case of the first candidate CAR-Ts (donors 1 and 2), the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed. It was found from these results that the first candidate CAR-Ts included first candidate CAR-Ts expressing CARs capable of binding to the A2/NY-ESO-1₁₅₇.

Next, first candidate CAR-Ts expressing CARs capable of binding to the A2/NY-ESO-1₁₅₇ were selected, and the CARs expressed by the first candidate CAR-Ts were identified. Specifically, the first candidate CAR-Ts were stained in the same manner as in (3) of Example 2 above. Then, cell sorting was performed on the first candidate CAR-Ts using a flow cytometer, and thus an NGFR⁺-CD8⁺-A2/NY-ESO-1₁₅₇ tetramer⁺ fraction or NGFR⁺-CD4⁺-A2/NY-ESO-1₁₅₇ tetramer⁺ fraction was collected as first candidate CAR-Ts expressing CARs capable of binding to the A2/NY-ESO-1₁₅₇. Then, scFvs were prepared in the same manner as in (5) of Example 1 above, except that newly collected first candidate CAR-Ts expressing CARs capable of binding to the A2/NY-ESO-1₁₅₇ were used instead of the first candidate CAR-Ts expressing CARs capable of binding to the A2/NY-ESO-1₁₅₇ described in (5) of Example (1) above, and the resulting scFvs were introduced into pMX/CAR expression vectors. Then, sequencing of the obtained pMX/CAR expression vectors into which the scFvs had been introduced was performed using the Sanger's method, and thus base sequences coding for CARs (a polynucleotide coding for L1-3M4E5 CAR (Sequence ID No. 149), a polynucleotide coding for L66-3M4E5 CAR (Sequence ID No. 151), a polynucleotide coding for L73-3M4E5 CAR (Sequence ID No. 152), a polynucleotide coding for L80-3M4E5 CAR (Sequence ID No. 153), a polynucleotide coding for L88-3M4E5 CAR (Sequence ID No. 154), a polynucleotide coding for L102-3M4E5 CAR (Sequence ID No. 155), and a polynucleotide coding for L124-3M4E5 CAR (Sequence ID No. 156)) were identified. Moreover, the amino acid sequences of the scFvs (L1-3M4E5 (Sequence ID No. 109), L66-3M4E5 (Sequence ID No. 111), L73-3M4E5 (Sequence ID No. 112), L80-3M4E5 (Sequence ID No. 113), L88-3M4E5 (Sequence ID No. 114), L102-3M4E5 (Sequence ID No. 115), and L124-3M4E5 (Sequence ID No. 116)) were identified based on the above-mentioned base sequences. Then, the CDRH1, CDRH2, and the CDRH3 in the new light-chain variable regions (LK) to (LQ), which are shown in Table 1B above, were identified based on the amino acid sequences of the scFvs.

(4) Analysis of First Candidate scFvs

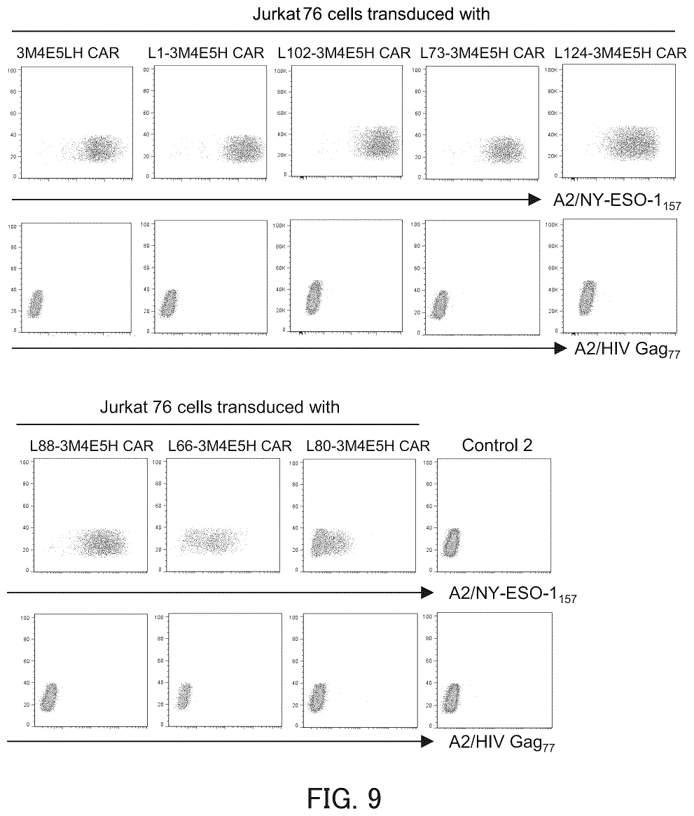

A culture supernatant containing GaLV-pseudotyped retroviruses was prepared in the same manner as in (3) of Example 1 above, except that the pMX/CAR expression vectors into which polynucleotides coding for the scFvs described in (3) of Example 2 above had been introduced instead of the polynucleotides coding for the scFvs in the expression vectors for expressing the hH-3M4E4L CAR library were used. Next, the pMX/CAR expression vectors into which the polynucleotides coding for the scFvs had been introduced were introduced into Jurkat 76 cells by adding the above-mentioned culture supernatant containing GaLV-pseudotyped retroviruses, and a CAR that included the L1-3M4E5, L66-3M4E5, L73-3M4E5, L80-3M4E5, L88-3M4E5, L102-3M4E5, or L124-3M4E5 as an scFv was expressed. The Jurkat 76 cells that had expressed the CARs were reacted with the PE-labeled anti-human NGFR mAb. After the above-mentioned reaction, Jurkat 76 cells expressing the CARs were purified using the anti-PE microbeads. The purified Jurkat 76 cells expressing the CARs were stained with the PE-labeled A2/NY-ESO-1₁₅₇ tetramer at a concentration of 5 μg/mL, and were then stained with the V450-labeled anti-human NGFR mAb. The stained Jurkat 76 cells expressing the CARs were analyzed by flow cytometry. A positive control was performed in the same manner, except that an expression vector that included a nucleic acid coding for the tagged 3M4E5LH CAR was used instead of the pMX/CAR expression vectors into which the scFvs had been introduced. A control 1 was performed in the same manner, except that the A2/HIV-Gag₇₇ tetramer was used instead of the A2/NY-ESO-1₁₅₇ tetramer. A control 2 was performed in the same manner, except that the pMX/CAR expression vectors into which the scFvs had been introduced were not introduced. FIG. 9 shows the results obtained when gating of NGFR⁺ cells was performed.

FIG. 9 shows dot plots showing the results of flow cytometry. In FIG. 9, the horizontal axes indicate the average fluorescence intensity of the A2 tetramer, and the vertical axes indicate the average side scatter (SSC) intensity. As shown in FIG. 9, the Jurkat 76 cells (control 2) into which the scFv-introduced pMX/CAR expression vectors had not been introduced did not include cells capable of binding to the A2/NY-ESO-1₁₅₇ or A2/HIV-Gag₇₇. Moreover, it could be confirmed that the 3M4E5LH CAR-Ts (3M4E5 CAR, positive control) were capable of binding to the A2/NY-ESO-1₁₅₇ but did not bind to the A2/HIV-Gag₇₇ because substantially all the cells were present in the A2 tetramer⁺ fraction when A2/NY-ESO-1₁₅₇ tetramer staining was performed, and the A2 tetramer⁺ fraction was not present when A2/HIV-Gag₇₇ tetramer staining was performed. Substantially all or some T cells expressing CARs that included L66-3M4E5, L73-3M4E5, L80-3M4E5, L88-3M4E5, L102-3M4E5, or L124-3M4E5 were present in the A2 tetramer⁺ fraction when A2/NY-ESO-1₁₅₇ tetramer staining was performed, and the A2 tetramer⁺ fraction was not present when A2/HIV-Gag₇₇ tetramer staining was performed. It was found from these results that the CARs that included L66-3M4E5, L73-3M4E5, L80-3M4E5, L88-3M4E5, L102-3M4E5, or L124-3M4E5 were capable of binding to the A2/NY-ESO-1₁₅₇ but did not bind to the A2/HIV-Gag₇₇. That is, it was found that the first screening method of the present invention could be used to screen scFvs that included new heavy-chain variable regions and new light-chain variable regions capable of binding to a target antigen. Moreover, since CAR-expressing T cells that were stained with the A2/NY-ESO-1₁₅₇ tetramer to different staining degrees were present, it was found that scFvs that included heavy-chain variable regions and light-chain variable regions having different avidity for a target antigen could be screened.

(5) Avidity of First Candidate scFvs

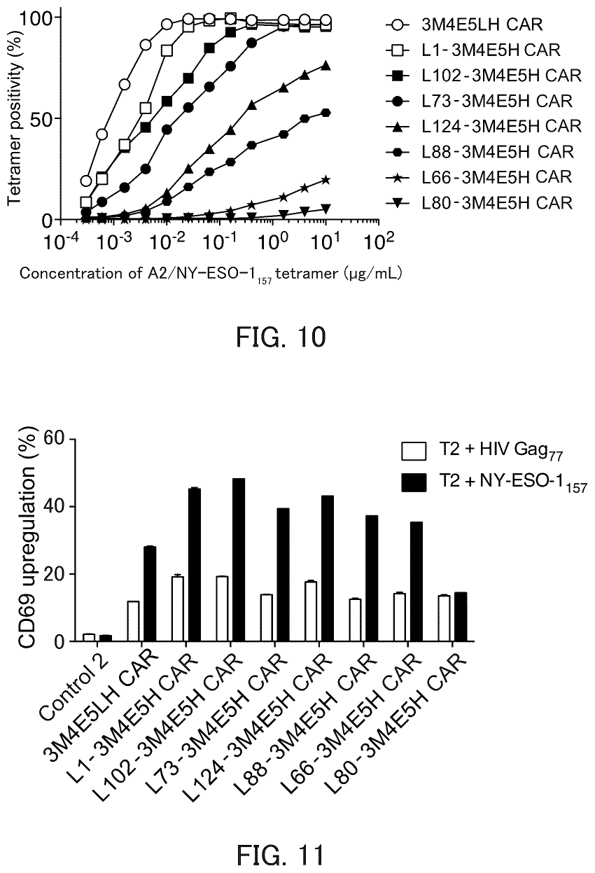

Next, flow cytometry analysis was performed in the same manner as in (6) above, except that the 3M4E5LH CAR-Ts and the Jurkat 76 cells expressing the CARs that included L1-3M4E5, L66-3M4E5, L73-3M4E5, L80-3M4E5, L88-3M4E5, L102-3M4E5, or L124-3M4E5, which had been obtained in (4) of Example 2 above, were stained with the PE-labeled A2/NY-ESO-1₁₅₇ tetramer at a predetermined concentration (0.0004194304, 0.001048576, 0.00262144, 0.0065536, 0.016384, 0.04096, 0.1024, 0.256, 0.64, 1.6, 4, or 10 μg/mL), and were then stained with the V450-labeled anti-human NGFR mAb. Then, the ratio of A2/NY-ESO-1₁₅₇ tetramer-positive cells was calculated for each A2/NY-ESO-1₁₅₇ tetramer concentration. FIG. 10 shows the results.

FIG. 10 is a graph indicating the ratios of A2/NY-ESO-1₁₅₇ tetramer-positive cells. In FIG. 10, the horizontal axis indicates the concentration of the A2/NY-ESO-1₁₅₇ tetramer, and the vertical axis indicates the ratio of A2/NY-ESO-1₁₅₇ tetramer-positive cells. As shown in FIG. 10, the ratios of the A2/NY-ESO-1₁₅₇ tetramer-positive cells in the CAR-Ts increased in a manner dependent on the concentration of the A2/NY-ESO-1₁₅₇ tetramer. It was necessary to increase the concentration of the A2/NY-ESO-1₁₅₇ tetramer in order to obtain, from the T cells expressing CARs that included L1-3M4E5, L66-3M4E5, L73-3M4E5, L80-3M4E5, L88-3M4E5, L102-3M4E5, or L124-3M4E5, the A2/NY-ESO-1₁₅₇ tetramer-positive cells at the same level as those from the 3M4E5LH CAR-Ts, and it was thus found that the scFvs in these CAR-Ts were different in avidity for the A2/NY-ESO-1₁₅₇ tetramer. That is, it was found that the first screening method of the present invention could be used to screen scFvs different in binding avidity for a target antigen.

(6) Functions of First Candidate scFvs

Next, it was examined if the CAR-Ts expressing L1-3M4E5, L66-3M4E5, L73-3M4E5, L80-3M4E5, L88-3M4E5, L102-3M4E5, or L124-3M4E5 as an scFv bound to the target antigen and were thus activated. Specifically, the CAR-Ts and the T2 cells were seeded in each well (96-well plate) such that the number of the CAR-Ts was 3×10⁵ and the number of the T2 cells was 5×10⁴, and then they were cultured together for 5 hours. Before use, the T2 cells (HLA-A*02:01-expressing cells) were pulsed with the NY-ESO-1₁₅₇₋₁₆₅ peptide at a predetermined concentration (0.0005, 0.0015, 0.004, 0.01, 0.04, 0.122, 0.366, 1.11, 3.33, or 10 μg/mL) (T2+NY-ESO-1₁₅₇). An RPMI1640 culture medium containing FCS at a concentration of 10% was used as the culture solution for the coculture. After the above-mentioned coculture, the CAR-Ts were collected, and were then stained with an FITC-labeled anti-human CD69 mAb (clone: FN50) and a V450-labeled anti-human NGFR mAb. The average fluorescence intensity of CD69 in NGFR⁺ cells from the stained CAR-Ts was measured using the flow cytometer. A positive control was performed in the same manner, except that an expression vector that included a nucleic acid coding for a tagged 3M4E5LH CAR was used instead of the pMX/H1-3M4E5L expression vector. A control 1 was performed in the same manner, except that T2 cells (T2+HIV Gag₇₇) pulsed with the HIV Gag₇₇₋₈₅ peptide instead of the NY-ESO-1₁₅₇₋₁₆₅ peptide were used. A control 2 was performed in the same manner, except that the expression vectors were not introduced. Then, the rates of an increase in CD69 expression in the samples were calculated using the average fluorescence intensity of CD69 in the control 2 as a standard. FIG. 11 shows the results.

FIG. 11 is a graph indicating changes in a CD69 expression level obtained when the peptide concentration was 10 μg/mL. In FIG. 11, the horizontal axis indicates the types of samples, and the vertical axis indicates the rate of an increase in CD69 expression. As shown in FIG. 11, the CD69 expression did not increase in the control 2. On the other hand, when the 3M4E5LH CAR-Ts (positive control) were stimulated with the T2+NY-ESO-1₁₅₇, it could be confirmed that the CD69 expression increased compared with the case of the stimulation with the T2+HIV Gag₇₇, and the CAR-Ts bound to the A2/NY-ESO-1₁₅₇ and were activated. When the CAR-Ts expressing L1-3M4E5, L66-3M4E5, L73-3M4E5, L80-3M4E5, L88-3M4E5, L102-3M4E5, or L124-3M4E5 as an scFv were stimulated with the T2+NY-ESO-1₁₅₇, the CD69 expression increased compared with the case of the stimulation with the T2+HIV Gag₇₇, and the CAR-Ts bound to the A2/NY-ESO-1₁₅₇ and were activated. Moreover, it was found that the first screening method of the present invention could be used to screen scFvs that were functional in CAR-T cells and had a higher specificity.

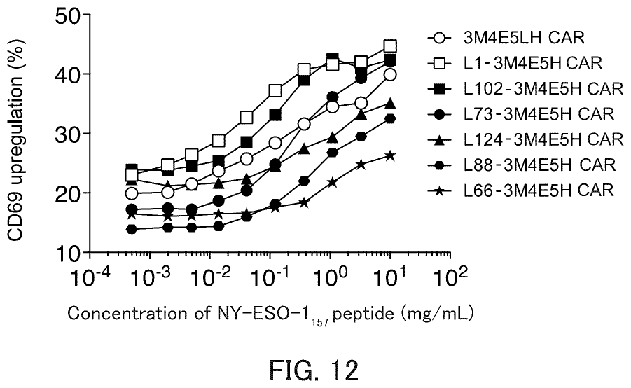

Next, the results of changes in CD69 expression in the case where the NY-ESO-1₁₅₇ peptide was serially diluted are shown in FIG. 12. FIG. 12 is a graph indicating changes in a CD69 expression level. In FIG. 12, the horizontal axis indicates the NY-ESO-1₁₅₇ peptide concentration, and the vertical axis indicates the rate of an increase in CD69 expression. As shown in FIG. 12, in the CAR-Ts, the CD69 expression increased in a manner dependent on the NY-ESO-1₁₅₇ peptide concentration. It was found that, in the CAR-Ts expressing L1-3M4E5 or L102-3M4E5 as the scFvs, the CD 69 expression increased more sharply and the specificity for the A2/NY-ESO-1₁₅₇ was higher compared with the 3M4E5LH CAR-Ts. That is, it was found that the first screening method of the present invention could be used to screen scFvs that were functional in CAR-T cells and had a higher specificity.

### Example 3

It was confirmed that the screening method of the present invention in which the CAR library of the present invention is used could be used to screen scFvs capable of binding to CD19.

### (1) Preparation of FMC63 CAR

A nucleic acid coding for an FMC63HL scFv or FMC63LH scFv was prepared in the same manner as in (1) of Example 1 above, except that the heavy-chain variable region and the light-chain variable region of a human CD19-specific antibody (clone: FMC63) were used instead of the heavy-chain variable region and the light-chain variable region of the A2/NY-ESO-1₁₅₇-specific antibody. Then, nucleic acids coding for a tagged FMC63HL CAR and a tagged FMC63LH CAR were obtained by adding a tag to the 3′ ends of the above-mentioned nucleic acids. The nucleic acids were introduced into pMX expression vectors.

FMC63HL scFv
(Sequence ID No. 201)
EVKLQESGPGLVAPSQSLSVTCTVSGVSLPDYGVSWIRQPPRKGLEW
LGVIWGSETTYYNSALKSRLTIIKDNSKSQVFLKMNSLQTDDTAIYY
CAKHYYYGGSYAMDYWGQGTSVTVSSGSTSGSGKPGSGEGSTKGDIQ
MTQTTSSLSASLGDRVTISCRASQDISKYLNWYQQKPDGTVKLLIYH
TSRLHSGVPSRFSGSGSGTDYSLTISNLEQEDIATYFCQQGNTLPYT
FGGGTKLEIT
Nucleic Acid Coding for FMC63HL scFv
(Sequence ID No. 202)
5′-GAAGTGAAACTGCAAGAGTCTGGCCCTGGACTGGTGGCCCCATC
TCAGTCTCTGAGCGTGACCTGTACAGTCAGCGGAGTGTCCCTGCCTG
ATTACGGCGTGTCCTGGATCAGACAGCCTCCTCGGAAAGGCCTGGAA
TGGCTGGGAGTGATCTGGGGCAGCGAGACAACCTACTACAACAGCGC
CCTGAAGTCCCGGCTGACCATCATCAAGGACAACTCCAAGAGCCAGG
TGTTCCTGAAGATGAACAGCCTGCAGACCGACGACACCGCCATCTAC
TATTGCGCCAAGCACTACTACTACGGCGGCAGCTACGCTATGGACTA
TTGGGGCCAGGGCACCAGCGTTACAGTGTCCTCGGGCTCTACAAGCG
GCTCTGGCAAGCCTGGATCTGGCGAGGGAAGCACCAAGGGCGATATC
CAGATGACCCAGACAACAAGCAGCCTGAGCGCCAGCCTGGGCGATAG
AGTGACCATCAGCTGTAGAGCCAGCCAGGACATCAGCAAGTACCTGA
ACTGGTATCAGCAGAAACCCGACGGCACCGTGAAGCTGCTGATCTAC
CACACCAGCAGACTGCACAGCGGCGTGCCAAGCAGATTTTCTGGCAG
CGGCTCTGGCACCGACTACAGCCTGACCATCTCCAACCTGGAACAAG
AGGATATCGCTACCTACTTCTGCCAGCAAGGCAACACCCTGCCTTAC
ACCTTTGGCGGAGGCACCAAGCTGGAAATCACA-3′
FMC63LH scFv
(Sequence ID No. 203)
DIQMTQTTSSLSASLGDRVTISCRASQDISKYLNWYQQKPDGTVKLL
IYHTSRLHSGVPSRFSGSGSGTDYSLTISNLEQEDIATYFCQQGNTL
PYTFGGGTKLEITGSTSGSGKPGSGEGSTKGEVKLQESGPGLVAPSQ
SLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYW
GQGTSVTVSS
Nucleic Acid Coding for FMC63LH scFv
(Sequence ID No. 204)
5′-GATATCCAGATGACCCAGACAACAAGCAGCCTGAGCGCCAGCCT
GGGCGATAGAGTGACCATCAGCTGTAGAGCCAGCCAGGACATCAGCA
AGTACCTGAACTGGTATCAGCAGAAACCCGACGGCACCGTGAAGCTG
CTGATCTACCACACCAGCAGACTGCACAGCGGCGTGCCAAGCAGATT
TTCTGGCAGCGGCTCTGGCACCGACTACAGCCTGACCATCTCCAACC
TGGAACAAGAGGATATCGCTACCTACTTCTGCCAGCAAGGCAACACC
CTGCCTTACACCTTTGGCGGAGGCACCAAGCTGGAAATCACAGGCTC
TACAAGCGGCTCTGGCAAGCCTGGATCTGGCGAGGGAAGCACCAAGG
GCGAAGTGAAACTGCAAGAGTCTGGCCCTGGACTGGTGGCCCCATCT
CAGTCTCTGAGCGTGACCTGTACAGTCAGCGGAGTGTCCCTGCCTGA
TTACGGCGTGTCCTGGATCAGACAGCCTCCTCGGAAAGGCCTGGAAT
GGCTGGGAGTGATCTGGGGCAGCGAGACAACCTACTACAACAGCGCC
CTGAAGTCCCGGCTGACCATCATCAAGGACAACTCCAAGAGCCAGGT
GTTCCTGAAGATGAACAGCCTGCAGACCGACGACACCGCCATCTACT
ATTGCGCCAAGCACTACTACTACGGCGGCAGCTACGCTATGGACTAT
TGGGGCCAGGGCACCAGCGTTACAGTGTCCTCG-3′

### (2) Preparation of First CAR Library

An hH-FMC63L scFv library was prepared by substituting the nucleic acid coding for the heavy-chain variable region on the 5′ side of the nucleic acids coding for the FMC63HL scFv described in (1) of Example 3 above with a library that included nucleic acids of the heavy-chain variable regions derived from human peripheral blood B cells. Then, a tagged hH-FMC63L CAR library was prepared in the same manner as in (1) of Example 1 above, except that the hH-FMC63L scFv library was used instead of the nucleic acid coding for the 3M4E5LH scFv. The tagged hH-FMC63L CAR library was introduced into pMX expression vectors to prepare expression vectors for expressing the hH-FMC63L CAR library. It should be noted that, regarding the heterogeneity of the heavy-chain variable regions in the hH-FMC63L CAR library, it was confirmed through restriction enzyme mapping and sequencing of the regions using the Sanger's method that the number of types of heavy-chain variable regions was about 1×10⁶.

### (3) Preparation of First CAR Library-Expressing T Cells (First Candidate CAR-Ts)

The hH-FMC63L CAR library was introduced into T cells in the same manner as in (3) of Example 1 above, except that the expression vectors for expressing the hH-FMC63L CAR library were used instead of the expression vectors for expressing the hH-3M4E4L CAR library. Then, the resulting T cells were purified and used as first candidate CAR-Ts.

### (4) Preparation of Target Antigen-Expressing Cells

Retroviruses were used to introduce a nucleic acid coding for human CD19 into K562 cells, and thus antigen-presenting cells (APCs-CD19) were obtained.

Nucleic Acid Coding for Human CD19
(Sequence ID No. 205)
5′-ATGCCACCTCCTCGCCTCCTCTTCTTCCTCCTCTTCCTCACCCC
CATGGAAGTCAGGCCCGAGGAACCTCTAGTGGTGAAGGTGGAAGAGG
GAGATAACGCTGTGCTGCAGTGCCTCAAGGGGACCTCAGATGGCCCC
ACTCAGCAGCTGACCTGGTCTCGGGAGTCCCCGCTTAAACCCTTCTT
AAAACTCAGCCTGGGGCTGCCAGGCCTGGGAATCCACATGAGGCCCC
TGGCCATCTGGCTTTTCATCTTCAACGTCTCTCAACAGATGGGGGGC
TTCTACCTGTGCCAGCCGGGGCCCCCCTCTGAGAAGGCCTGGCAGCC
TGGCTGGACAGTCAATGTGGAGGGCAGCGGGGAGCTGTTCCGGTGGA
ATGTTTCGGACCTAGGTGGCCTGGGCTGTGGCCTGAAGAACAGGTCC
TCAGAGGGCCCCAGCTCCCCTTCCGGGAAGCTCATGAGCCCCAAGCT
GTATGTGTGGGCCAAAGACCGCCCTGAGATCTGGGAGGGAGAGCCTC
CGTGTCTCCCACCGAGGGACAGCCTGAACCAGAGCCTCAGCCAGGAC
CTCACCATGGCCCCTGGCTCCACACTCTGGCTGTCCTGTGGGGTACC
CCCTGACTCTGTGTCCAGGGGCCCCCTCTCCTGGACCCATGTGCACC
CCAAGGGGCCTAAGTCATTGCTGAGCCTAGAGCTGAAGGACGATCGC
CCGGCCAGAGATATGTGGGTAATGGAGACGGGTCTGTTGTTGCCCCG
GGCCACAGCTCAAGACGCTGGAAAGTATTATTGTCACCGTGGCAACC
TGACCATGTCATTCCACCTGGAGATCACTGCTCGGCCAGTACTATGG
CACTGGCTGCTGAGGACTGGTGGCTGGAAGGTCTCAGCTGTGACTTT
GGCTTATCTGATCTTCTGCCTGTGTTCCCTTGTGGGCATTCTTCATC
TTCAAAGAGCCCTGGTCCTGAGGAGGAAAAGAAAGCGAATGACTGAC
CCCACCAGGAGATTCTTCAAAGTGACGCCTCCCCCAGGAAGCGGGCC
CCAGAACCAGTACGGGAACGTGCTGTCTCTCCCCACACCCACCTCAG
GCCTCGGACGCGCCCAGCGTTGGGCCGCAGGCCTGGGGGGCACTGCC
CCGTCTTATGGAAACCCGAGCAGCGACGTCCAGGCGGATGGAGCCTT
GGGGTCCCGGAGCCCGCCGGGAGTGGGCCCAGAAGAAGAGGAAGGGG
AGGGCTATGAGGAACCTGACAGTGAGGAGGACTCCGAGTTCTATGAG
AACGACTCCAACCTTGGGCAGGACCAGCTCTCCCAGGATGGCAGCGG
CTACGAGAACCCTGAGGATGAGCCCCTGGGTCCTGAGGATGAAGACT
CCTTCTCCAACGCTGAGTCTTATGAGAACGAGGATGAAGAGCTGACC
CAGCCGGTCGCCAGGACAATGGACTTCCTGAGCCCTCATGGGTCAGC
CTGGGACCCCAGCCGGGAAGCAACCTCCCTGGGGTCCCAGTCCTATG
AGGATATGAGAGGAATCCTGTATGCAGCCCCCCAGCTCCGCTCCATT
CGGGGCCAGCCTGGACCCAATCATGAGGAAGATGCAGACTCTTATGA
GAACATGGATAATCCCGATGGGCCAGACCCAGCCTGGGGAGGAGGGG
GCCGCATGGGCACCTGGAGCACCAGG-3′
(5) First Screening Method

Screening was performed using the first candidate CAR-Ts described in (3) of Example 3 above and the APCs-CD19 prepared in (4) of Example 3 above. Specifically, the first candidate CAR-Ts and the APCs-CD19 were seeded in each well (24-well plate) such that the number of the first candidate CAR-Ts was 2×10⁶ and the number of the APCs-CD19 was 1×10⁵, and then they were cultured together for 7 days. Before use, the APCs-CD19 were treated with 20 Gy of γ rays. An RPMI1640 culture medium containing gentamicin at a concentration of 50 μg/mL, human AB serum (manufactured by Sigma) at a concentration of 10%, human IL-2 (manufactured by Roche) at a concentration of 10 IU/mL, and human IL-15 (manufactured by PeproTech) at a concentration of 10 ng/mL was used as the culture medium used for the above-mentioned coculture of the first candidate CAR-Ts and the APCs-CD19. After the coculture, the first candidate CAR-Ts were collected, and coculture of the first candidate CAR-Ts and the APCs-CD19 was performed two more times (three times in total) in the same conditions. Thus, the first candidate CAR-Ts expressing the hH-FMC63L CARs capable of binding to human CD19 was enriched.

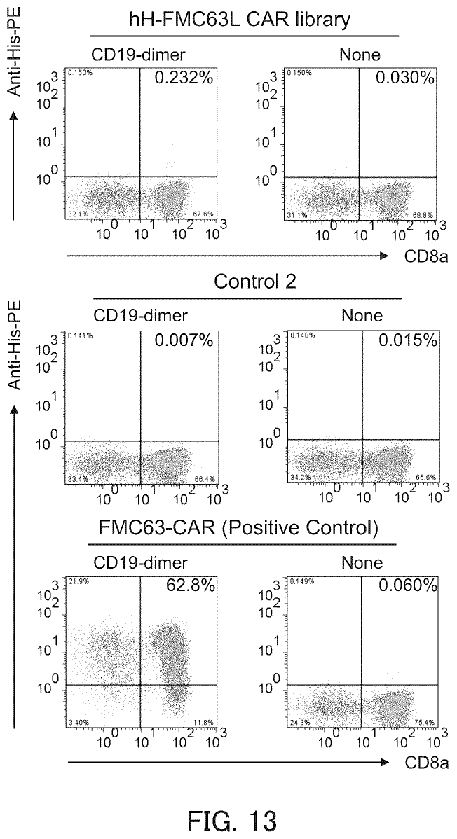

Next, after the enrichment of the first candidate CAR-Ts, the first candidate CAR-Ts were collected and stained with a PE-labeled CD19 dimer at a concentration of 20 μg/mL. The PE-labeled CD19 dimer was prepared using a soluble CD19, which is human CD19 that includes a His-tag added to the C terminus of the extracellular domain thereof via an SGSG linker. Specifically, the PE-labeled CD19 dimer was prepared by mixing the soluble CD19 and a PE-labeled anti-His mAb (clone: GG11-8F3.5.1) such that the molar ratio therebetween was 2:1. After the above-mentioned staining, the T cells were further stained with a PC5-labeled anti-human CD8a mAb (clone: B9.11), an FITC-labeled anti-human CD4 mAb (clone: OKT4), and a V450-labeled anti-human NGFR mAb (clone: C40-1457). Then, regarding the first candidate CAR-Ts that had been subjected to dimer staining, it was confirmed using the flow cytometer if first candidate CAR-Ts expressing CARs capable of binding to CD19 were present. A positive control was performed in the same manner, except that an expression vector that included a nucleic acid coding for the tagged FMC63HL CAR was used instead of the expression vectors for expressing the hH-FMC63L CAR library. It should be noted that, even when an expression vector that included a nucleic acid coding for the tagged FMC63LH CAR was used, the results were the same as those from the case where the expression vector that included a nucleic acid coding for the tagged FMC63HL CAR was used. A control 1 was performed in the same manner, except that CD19 dimer staining was not performed. A control 2 was performed in the same manner, except that the expression vectors for expressing the hH-FMC63L CAR library were not introduced. FIG. 13 shows the results obtained when gating of FSC⁺-SSC⁺-NGFR⁺ cells was performed.

FIG. 13 shows dot plots showing the results of flow cytometry. In FIG. 13, the horizontal axes indicate the average fluorescence intensity of CD8a, and the vertical axes indicate the average fluorescence intensity of the CD19 dimer. As shown in FIG. 13, the T cells (control 2) into which the expression vectors for expressing the hH-FMC63 CAR library had not been introduced did not include T cells capable of binding to the CD19 dimer. Moreover, it could be confirmed that the CAR-Ts (FMC63-CAR, positive control) expressing the FMC63-CAR were capable of binding to the CD19 dimer because a CD8⁺-CD19⁺ dimer fraction (the upper right fraction in each dot plot) was present when CD19 dimer staining was performed, and the CD8⁺-CD19 dimer⁺ fraction was not present when CD19 dimer staining was not performed. Furthermore, in the case of the first candidate CAR-Ts, the CD8⁺-CD19 dimer⁺ fraction was present when CD19 dimer staining was performed, and the CD8⁺-CD19 dimer⁺ fraction was not present when CD19 dimer staining was not performed. It was found from these results that the first candidate CAR-Ts included first candidate CAR-Ts expressing CARs capable of binding to the CD19 dimer. As a result, it was found that the first screening method of the present invention could be used to screen scFvs capable of binding to a target antigen. It should be noted that, although data are not shown, it was confirmed that the CD69 expression increased in cells included in the CD8⁺-CD19 dimer⁺ fraction compared with cells included in the CD19 dimer fraction, and CARs expressed in the cells included in the CD8⁺-CD19 dimer⁺ fraction had a T cell-activating function.

### Example 4

It was confirmed that CARs obtained using the screening method of the present invention in which the CAR library of the present invention is used had high specificity for a target antigen.

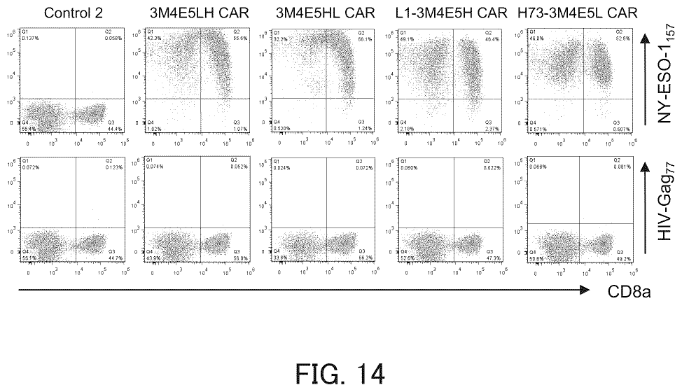

A culture supernatant containing GaLV-pseudotyped retroviruses was prepared in the same manner as in (3) of Example 1 above, except that a pMX/CAR expression vector into which a polynucleotide coding for L1-3M4E5H or H73-3M4E5L as an scFv (antigen-binding domain) had been introduced (respectively referred to as an L1-3M4E5H expression vector and an H73-3M4E5L expression vector) was used instead of the expression vectors for expressing the hH-3M4E4L CAR library. Next, T cells were infected with the retroviruses in the same manner as in (3) of Example 1 above, except that the obtained GaLV-pseudotyped retroviruses were used. Thus, the L1-3M4E5H expression vector or H73-3M4E5L expression vector was introduced into the T cells. The infection with the retroviruses and the introduction of the L1-3M4E5H expression vector or H73-3M4E5L expression vector were performed in the same manner five more times (six times in total). The resulting T cells were stained with the PE-labeled A2/NY-ESO-1₁₅₇ tetramer at a concentration of 20 μg/mL. After the above-mentioned staining, the T cells were further stained with a PC5-labeled anti-human CD8a mAb (clone: B9.11), an FITC-labeled anti-human CD4 mAb (clone: OKT4), and a V450-labeled anti-human NGFR mAb (clone: C40-1457). The concentrations of these antibodies were about 10 μg/mL. Then, it was confirmed using the flow cytometer if the T cells that had been subjected to tetramer staining included T cells expressing CARs capable of binding to the A2/NY-ESO-1₁₅₇. A positive control was performed in the same manner, except that an expression vector that included a nucleic acid coding for the tagged 3M4E5HL CAR or tagged 3M4E5LH CAR was used instead of the expression vectors for expressing the hH-3M4E4L CAR library. A control 1 was performed in the same manner, except that the A2/HIV-Gag₇₇ tetramer was used instead of the A2/NY-ESO-1₁₅₇ tetramer. A control 2 was performed in the same manner, except that the expression vectors for expressing the hH-3M4E4L CAR library were not introduced. FIG. 14 shows the results obtained when gating of FSC⁺-SSC⁺-NGFR⁺ cells was performed.

FIG. 14 shows dot plots showing the results of flow cytometry. In FIG. 14, the horizontal axes indicate the average fluorescence intensity of CD8a, and the vertical axes indicate the average fluorescence intensity of the A2 tetramer. As shown in FIG. 14, in the control 2, T cells capable of binding to the A2/NY-ESO-1₁₅₇ or A2/HIV-Gag₇₇ were not present. Moreover, it could be confirmed that the CAR-Ts (3M4E5HL CAR or 3M4E5LH CAR, positive control) expressing the 3M4E5HL CAR or 3M4E5LH CAR were capable of binding to the A2/NY-ESO-1₁₅₇ but did not bind to the A2/HIV-Gag₇₇ because a CD8⁺-A2 tetramer⁺ fraction (the upper right fraction in each dot plot) was present when A2/NY-ESO-1₁₅₇ tetramer staining was performed, and the CD8⁺-A2 tetramer⁺ fraction was not present when A2/HIV-Gag₇₇ tetramer staining was performed. Furthermore, in the case of the L1-3M4E5H CAR-Ts or H73-3M4E5L CAR-Ts, the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed.

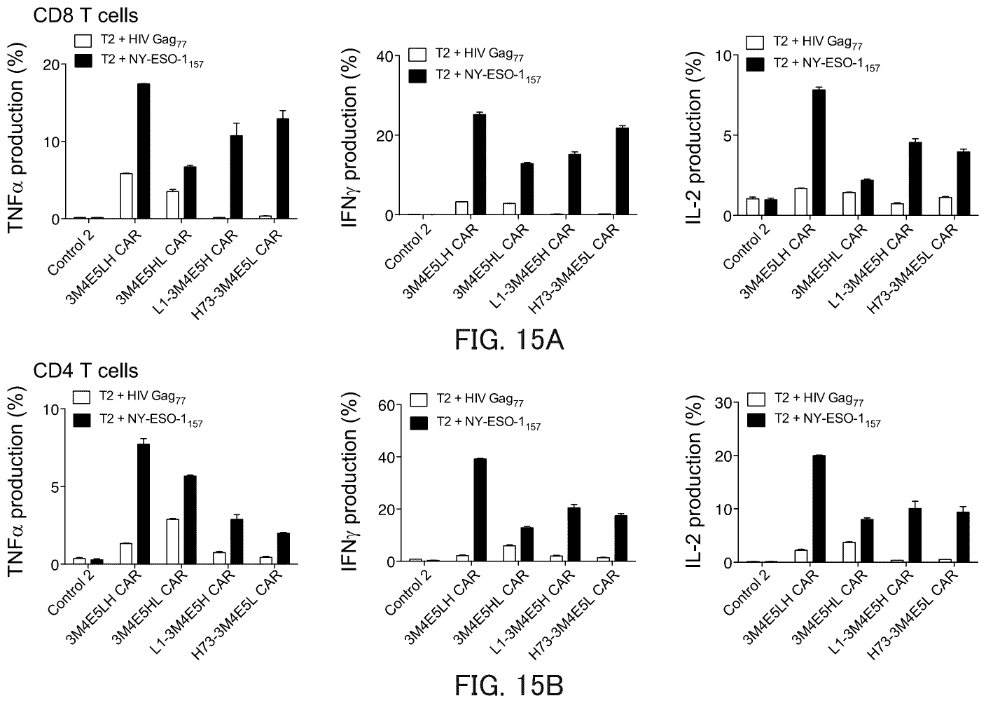

Next, it was examined if the T cells that had been infected with the retroviruses bound to the target antigen, and were thus activated to produce cytokines. Specifically, after the infection of T cells (CAR-Ts) with the retroviruses were repeated six times, the T cells and T2 cells were seeded in each well (96-well plate) such that the number of the T cells was 3×10⁵ and the number of the T2 cells was 5×10⁴, and then they were cultured together for 5 hours. Before use, the T2 cells (HLA-A*02:01-expressing cells) were pulsed with the NY-ESO-1₁₅₇ peptide at a concentration of 10 μg/mL (T2+NY-ESO-1₁₅₇). An RPMI1640 culture medium containing FCS at a concentration of 10% was used as the culture solution for the coculture. After the above-mentioned coculture, the T cells were collected, fixed with paraformaldehyde, subjected to permeabilization, and stained with an APC-labeled anti-human IL-2 mAb (clone: MQ1-17H12), a PE-labeled anti-human TNFα mAb (clone: Mab11), a PC7-labeled anti-human IFN-γ mAb (clone: B27), an FITC-labeled anti-human CD4 mAb (clone: OKT4), a PC5-labeled anti-human CD8a mAb (clone: B9.11), and a V450-labeled anti-human NGFR mAb (clone: C40-1457). After the CAR-Ts were stained, the amounts of TNF-α, IFN-γ, and IL-2 produced by CD8-positive cells or CD4-positive cells in the CAR-Ts (NGFR-positive fraction) were measured using the flow cytometer. A positive control was performed in the same manner, except that an expression vector that included a nucleic acid coding for the 3M4E5HL or 3M4E5LH as an scFv was used. A control 1 was performed in the same manner, except that the A2/HIV-Gag₇₇ peptide was used instead of the NY-ESO-1₁₅₇ peptide. A control 2 was performed in the same manner, except that the expression vectors for expressing the hH-3M4E4L CAR library were not introduced. Then, the rates of an increase in cytokine production in the samples were calculated using the cytokine production amount in the control 2 as a standard. FIG. 15(A) and FIG. 15(B) show the results.

FIG. 15(A) and FIG. 15(B) show graphs indicating changes in a cytokine production amount. FIG. 15(A) shows the results from CD8-positive T cells, and FIG. 15(B) shows the results from CD4-positive T cells. In FIG. 15(A) and FIG. 15(B), the horizontal axes indicate the types of samples (CARs), and the vertical axes indicate the rate of an increase in cytokine production. As shown in FIG. 15(A) and FIG. 15(B), the cytokine production did not increase in the control 2. On the other hand, when the 3M4E5LH CAR-Ts or 3M4E5HL CAR-Ts (positive control) were stimulated with the T2+NY-ESO-1₁₅₇, it could be confirmed that the cytokine production amount increased compared with the case of the stimulation with the T2+HIV Gag₇₇, and the cytokines bound to the A2/NY-ESO-1₁₅₇ to activate the CAR-Ts. When the 3M4E5HL CAR-Ts or 3M4E5LH CAR-Ts were stimulated with the T2+NY-ESO-1₁₅₇, the cytokine production increased compared with the case of the stimulation with the T2+HIV Gag₇₇, and the cytokines bound to the A2/NY-ESO-1₁₅₇ to activate the CAR-Ts, leading to the production of cytokines. Furthermore, when the 3M4E5HL CAR-Ts or 3M4E5LH CAR-Ts were stimulated with the T2+HIV Gag₇₇, the cytokine production amount was smaller than or equal to that in the case of the control 2, and was smaller than that in the case of the positive control. It was found from these results that the CARs obtained using the screening method of the present invention in which the CAR library of the present invention is used had high specificity for a target antigen.

### Example 5

It was confirmed that CARs obtained using the screening method of the present invention in which the CAR library of the present invention is used differed from antibodies capable of binding to a target antigen used for screening in antigen recognition.

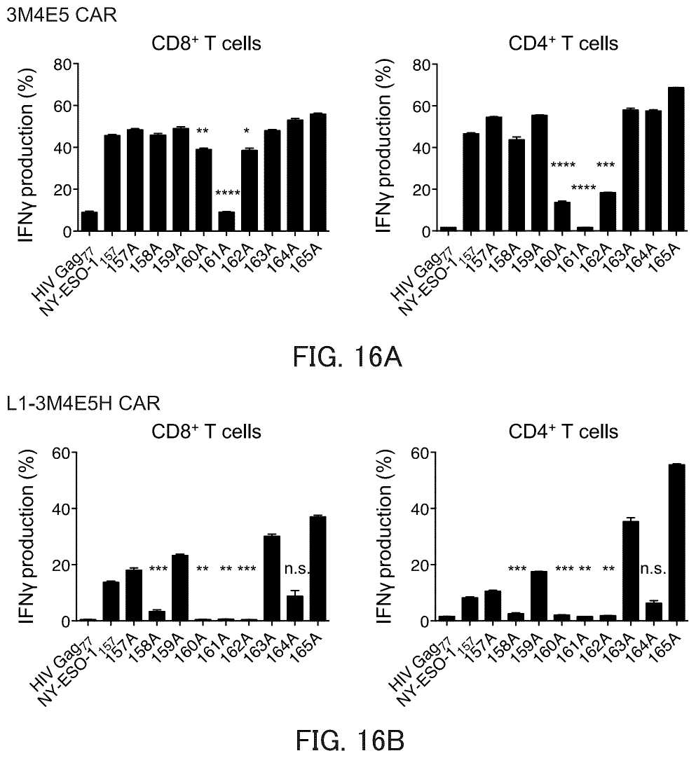

CD8-positive T cells and CD4-positive T cells expressing the L1-3M4E5H CAR were produced in the same manner as in Example 4 above. Next, the T cells and T2 cells were seeded in each well (96-well plate) such that the number of the T cells was 3×10⁵ and the number of the T2 cells was 5×10⁴, and then they were cultured together for 5 hours. Before use, the T2 cells (HLA-A*02:01-expressing cells) were pulsed with the NY-ESO-1₁₅₇ peptide or a mutant peptide of the NY-ESO-1₁₅₇ peptide at a concentration of 10 μg/mL (T2+NY-ESO-1₁₅₇). The mutant peptide is a peptide obtained by substituting one of the amino acids in the amino acid sequence of the NY-ESO-1₁₅₇ peptide with alanine. After the above-mentioned coculture, the culture supernatant was collected, and the concentration of IFN-γ in the CAR-Ts was measured in the same manner as in Example 4 above. A control was performed in the same manner, except that CD8-positive T cells or CD4-positive T cells expressing the 3M4E5LH CAR were used. A control 1 was performed in the same manner, except that the A2/HIV-Gag₇₇ peptide was used instead of the A2/NY-ESO-1₁₅₇ peptide. A control 2 was performed in the same manner, except that CD8-positive T cells or CD4-positive T cells into which the expression vectors had not been introduced were used. Then, the rates of an increase in cytokine production in the samples were calculated using the cytokine production amount in the control 2 as a standard. FIG. 16(A) and FIG. 16(B) show the results.

FIG. 16(A) and FIG. 16(B) show graphs indicating changes in a cytokine production amount. FIG. 16(A) shows the results from T cells expressing the 3M4E5LH CAR, and FIG. 16(B) shows the results from T cells expressing the L1-3M4E5H CAR. In FIGS. 16(A) and 16(B), the horizontal axes indicate the types of peptides, and the vertical axes indicate the changes in a cytokine production amount. The 3M4E5LH CAR recognized the amino acids at positions 160 to 162 of the NY-ESO-1₁₅₇ peptide as shown in FIG. 16(A), whereas the L1-3M4E5H CAR recognized the amino acids at positions 158 and 160 to 162 of the NY-ESO-1₁₅₇ peptide as shown in FIG. 16(B). It was found from these results that CARs obtained using the screening method of the present invention in which the CAR library of the present invention is used differed from antibodies capable of binding to a target antigen used for screening in antigen recognition.

### Example 6

It was confirmed that CARs obtained using the screening method of the present invention in which the CAR library of the present invention is used maintained the antigen recognition even when the heavy-chain variable region and the light-chain variable region were arranged in the reverse order.

A 3M4E5H-L1 expression vector was produced by arranging the polynucleotide coding for the heavy-chain variable region and the polynucleotide coding for the light-chain variable region of the L1-3M4E5H expression vector in the reverse order.

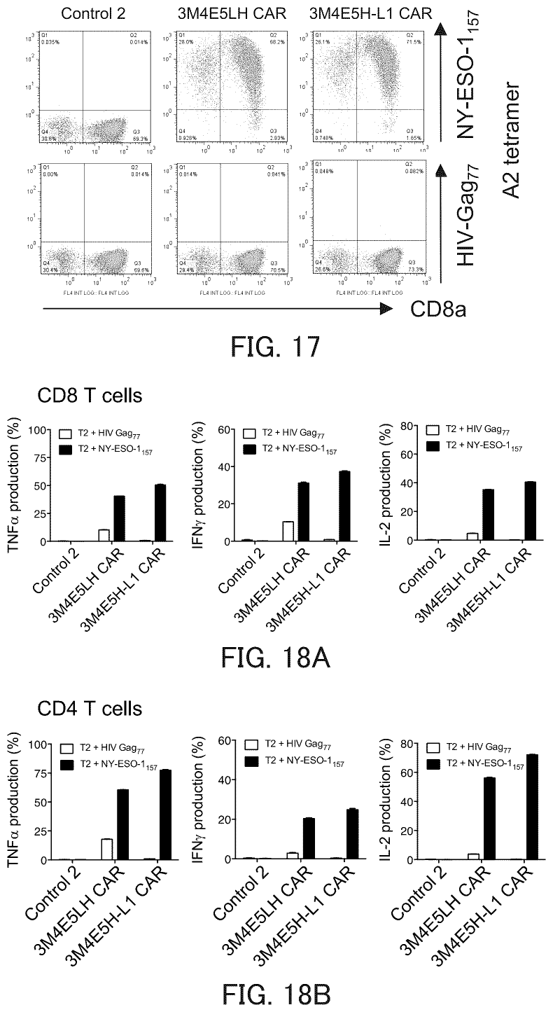

A culture supernatant containing GaLV-pseudotyped retroviruses was prepared in the same manner as in (3) of Example 1 above, except that the 3M4E5H-L1 expression vector was used instead of the expression vectors for expressing the hH-3M4E4L CAR library. Next, T cells were infected with the retroviruses in the same manner as in (3) of Example 1 above, except that the obtained GaLV-pseudotyped retroviruses were used. Thus, the 3M4E5H-L1 expression vector was introduced into the T cells. The infection with the retroviruses and the introduction of the 3M4E5H-L1 expression vector were performed in the same manner five more times (six times in total). The resulting T cells were stained with the PE-labeled A2/NY-ESO-1₁₅₇ tetramer at a concentration of 20 μg/mL. After the above-mentioned staining, the T cells were further stained with a PC5-labeled anti-human CD8a mAb (clone: B9.11), an FITC-labeled anti-human CD4 mAb (clone: OKT4), and a V450-labeled anti-human NGFR mAb (clone: C40-1457). The concentrations of these antibodies were about 10 μg/mL. Then, it was confirmed using the flow cytometer if the T cells that had been subjected to tetramer staining included T cells expressing CARs capable of binding to the A2/NY-ESO-1₁₅₇. A positive control was performed in the same manner, except that an expression vector that included a nucleic acid coding for the tagged 3M4E5LH CAR was used instead of the expression vectors for expressing the hH-3M4E4L CAR library. A control 1 was performed in the same manner, except that the A2/HIV-Gag₇₇ tetramer was used instead of the A2/NY-ESO-1₁₅₇ tetramer. A control 2 was performed in the same manner, except that the expression vectors for expressing the hH-3M4E4L CAR library were not introduced. FIG. 17 shows the results obtained when gating of FSC⁺-SSC⁺-NGFR⁺ cells was performed.

FIG. 17 shows dot plots showing the results of flow cytometry. In FIG. 17, the horizontal axes indicate the average fluorescence intensity of CD8a, and the vertical axes indicate the average fluorescence intensity of the A2 tetramer. As shown in FIG. 17, in the control 2, T cells capable of binding to the A2/NY-ESO-1₁₅₇ or A2/HIV-Gag₇₇ were not present. Moreover, it could be confirmed that the CAR-Ts (3M4E5LH CAR, positive control) expressing the 3M4E5LH CAR were capable of binding to the A2/NY-ESO-1₁₅₇ but did not bind to the A2/HIV-Gag₇₇ because a CD8⁺-A2 tetramer⁺ fraction (the upper right fraction in each dot plot) was present when A2/NY-ESO-1₁₅₇ tetramer staining was performed, and the CD8⁺-A2 tetramer⁺ fraction was not present when A2/HIV-Gag₇₇ tetramer staining was performed. Furthermore, in the case of the CAR-Ts expressing the 3M4E5H-L1, the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed.

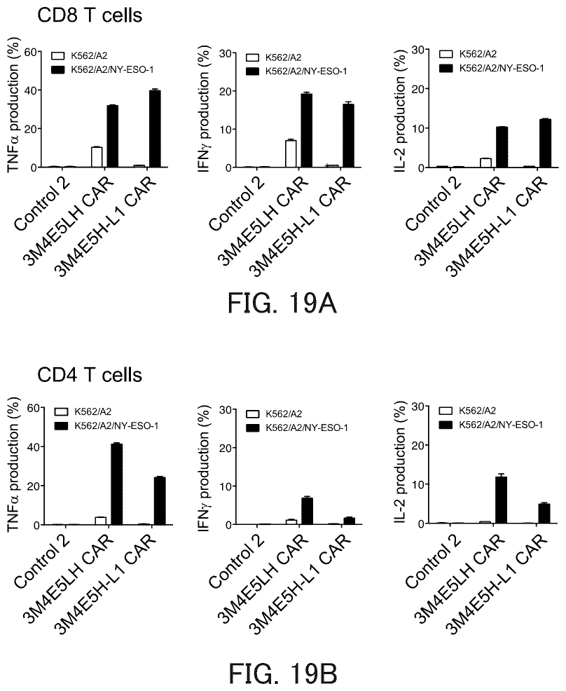

Next, it was examined if the T cells that had been infected with the retroviruses bound to the target antigen, and were thus activated to produce cytokines. Specifically, after the infection of T cells (CAR-Ts) with the retroviruses were repeated six times, the T cells, and T2 cells or APCs prepared in (4) of Example 1 above were seeded in each well (96-well plate) such that the number of the T cells was 3×10⁵ and the number of the T2 cells or APCs was 5×10⁴, and then they were cultured together for 5 hours. Before use, the T2 cells (HLA-A*02:01-expressing cells) and APCs were pulsed with the NY-ESO-1₁₅₇ peptide at a concentration of 10 μg/mL (T2+NY-ESO-1₁₅₇ and K562/A2/NY-ESO-1). An RPMI1640 culture medium containing FCS at a concentration of 10% was used as the culture solution for the coculture. After the above-mentioned coculture, the T cells were collected, fixed with paraformaldehyde, subjected to permeabilization, and stained with an APC-labeled anti-human IL-2 mAb (clone: MQ1-17H12), a PE-labeled anti-human TNFα mAb (clone: Mab11), a PC7-labeled anti-human IFN-γ antibody (clone: B27), an FITC-labeled anti-human CD4 mAb (clone: OKT4), a PC5-labeled anti-human CD8a mAb (clone: B9.11), and a V450-labeled anti-human NGFR mAb (clone: C40-1457). After the CAR-Ts were stained, the amounts of TNF-α, IFN-γ, and IL-2 produced by CD8-positive cells or CD4-positive cells in the CAR-Ts (NGFR-positive fraction) were measured using the flow cytometer. A positive control was performed in the same manner, except that an expression vector that included a nucleic acid coding for the 3M4E5LH as an scFv was used. When the T2 cells were used, a control 1 was performed in the same manner, except that the HIV-Gag₇₇ peptide was used instead of the NY-ESO-1₁₅₇ peptide. When the APCs (K562/A2/NY-ESO-1) were used, a control 1 was performed in the same manner, except that K562/A2 into which the NY-ESO-1 gene had not been introduced was used. A control 2 was performed in the same manner, except that the expression vectors for expressing the hH-3M4E4L CAR library were not introduced. Then, the rates of an increase in cytokine production in the samples were calculated using the cytokine production amount in the control 2 as a standard. FIGS. 18 and 19 show the results.

FIG. 18(A) and FIG. 18(B) show the results obtained when the T2 cells were used. FIG. 18(A) and FIG. 18(B) show graphs indicating changes in a cytokine production amount. FIG. 18(A) shows the results from CD8-positive T cells, and FIG. 18(B) shows the results from CD4-positive T cells. In FIG. 18(A) and FIG. 18(B), the horizontal axes indicate the types of samples (CARs), and the vertical axes indicate the rate of an increase in cytokine production. As shown in FIG. 18(A) and FIG. 18(B), the cytokine production did not increase in the control 2. On the other hand, when the 3M4E5LH CAR-Ts (positive control) were stimulated with the T2+NY-ESO-1₁₅₇, it could be confirmed that the cytokine production amount increased compared with the case of the stimulation with the T2+HIV Gag₇₇, and the cytokines bound to the A2/NY-ESO-1₁₅₇ to activate the CAR-Ts. When the 3M4E5H-L1 CAR-Ts were stimulated with the T2+NY-ESO-1₁₅₇, the cytokine production increased compared with the case of the stimulation with the T2+HIV Gag₇₇, and the cytokines bound to the A2/NY-ESO-1₁₅₇ to activate the CAR-Ts, leading to the production of cytokines. Furthermore, when the 3M4E5H-L1 CAR-Ts were stimulated with the T2+HIV Gag₇₇, the cytokine production amount was smaller than or equal to that in the case of the control 2, and was smaller than that in the case of the positive control.

FIG. 19(A) and FIG. 19(B) show the results obtained when the APCs were used. FIG. 19(A) and FIG. 19(B) show graphs indicating changes in a cytokine production amount. FIG. 19(A) shows the results from CD8-positive T cells, and FIG. 19(B) shows the results from CD4-positive T cells. In FIG. 19(A) and FIG. 19(B), the horizontal axes indicate the types of samples (CARs), and the vertical axes indicate the rate of an increase in cytokine production. As shown in FIG. 19(A) and FIG. 19(B), the cytokine production did not increase in the control 2. On the other hand, when the 3M4E5LH CAR-Ts (positive control) were stimulated with the K562/A2/NY-ESO-1, it could be confirmed that the cytokine production amount increased compared with the case of the stimulation with the K562/A2, and the cytokines bound to the K562/A2/NY-ESO-1 to activate the CAR-Ts. When the 3M4E5H-L1 CAR-Ts were stimulated with the K562/A2/NY-ESO-1, the cytokine production increased compared with the case of the stimulation with the K562/A2, and the cytokines bound to the K562/A2/NY-ESO-1 to activate the CAR-Ts, leading to the production of cytokines. Furthermore, when the 3M4E5H-L1 CAR-Ts were stimulated with the K562/A2, the cytokine production amount was equal to that in the case of the control 2, and was smaller than that in the case of the positive control. It was found from these results that the CARs obtained using the screening method of the present invention in which the CAR library of the present invention is used could maintain the antigen recognition even when the heavy-chain variable region and the light-chain variable region were arranged in the reverse order.

### Example 7

It was confirmed that CARs obtained using the screening method of the present invention in which the CAR library of the present invention is used differed from antibodies capable of binding to a target antigen used for screening in antigen recognition, and could be screened while the crossreactivity of scFvs in the CARs were controlled.

### (1) Antigen Recognition

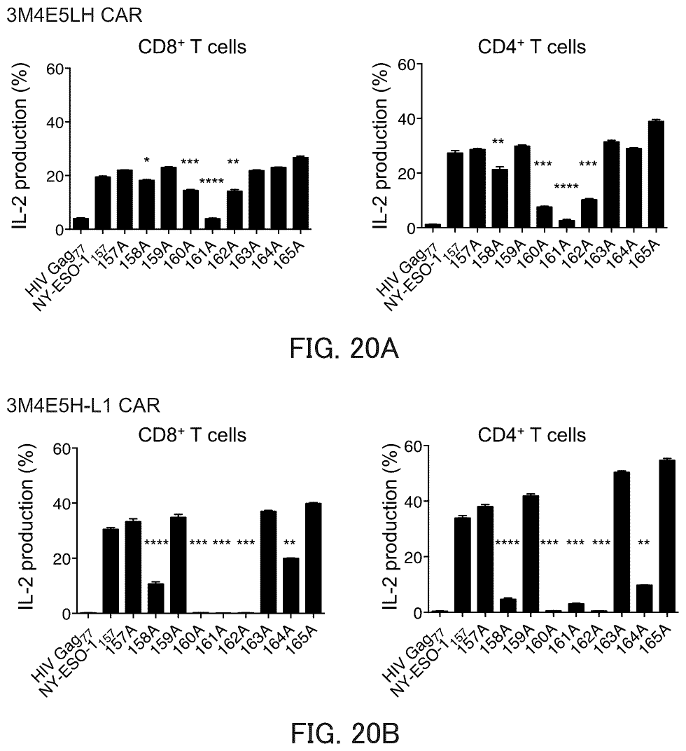

CD8-positive T cells and CD4-positive T cells expressing the 3M4E5H-L1 CAR were produced in the same manner as in Example 6 above. Next, the T cells and T2 cells were seeded in each well (96-well plate) such that the number of the T cells was 3×10⁵ and the number of the T2 cells was 5×10⁴, and then they were cultured together for 5 hours. Before use, the T2 cells (HLA-A*02:01-expressing cells) were pulsed with the NY-ESO-1₁₅₇ peptide or a mutant peptide of the NY-ESO-1₁₅₇ peptide at a concentration of 10 μg/mL (T2+NY-ESO-1₁₅₇). The mutant peptide is a peptide obtained by substituting one of the amino acids in the amino acid sequence of the NY-ESO-1₁₅₇ peptide with alanine. After the above-mentioned coculture, the culture supernatant was collected, and the concentration of IL-2 in the CAR-Ts was measured in the same manner as in Example 4 above. A control was performed in the same manner, except that CD8-positive T cells or CD4-positive T cells expressing the 3M4E5LH CAR were used. A control 1 was performed in the same manner, except that the A2/HIV-Gag₇₇ peptide was used instead of the A2/NY-ESO-1₁₅₇ peptide. A control 2 was performed in the same manner, except that CD8-positive T cells or CD4-positive T cells into which the expression vectors had not been introduced were used. Then, the rates of an increase in cytokine production in the samples were calculated using the cytokine production amount in the control 2 as a standard. FIG. 20(A) and FIG. 20(B) show the results.

FIG. 20(A) and FIG. 20(B) show graphs indicating changes in a cytokine production amount. FIG. 20(A) shows the results from T cells expressing the 3M4E5LH CAR, and FIG. 20(B) shows the results from T cells expressing the 3M4E5H-L1 CAR. In FIGS. 20(A) and 20(B), the horizontal axes indicate the types of peptides, and the vertical axes indicate the changes in a cytokine production amount. The 3M4E5LH CAR mainly recognized the amino acids at positions 160 to 162 of the NY-ESO-1₁₅₇ peptide as shown in FIG. 20(A), whereas the 3M4E5H-L1 CAR recognized the amino acids at positions 158, 160 to 162, and 164 of the NY-ESO-1₁₅₇ peptide as shown in FIG. 20(B). It was found from these results that CARs obtained using the screening method of the present invention in which the CAR library of the present invention is used differed from antibodies capable of binding to a target antigen used for screening in antigen recognition.

### (2) Crossreactivity

As shown in (1) of Example 7 above, the 3M4E5LH CAR mainly recognized the amino acids at positions 160 to 162 of the NY-ESO-1₁₅₇ peptide, whereas the 3M4E5H-L1 CAR recognized the amino acids at positions 158, 160 to 162, and 164 of the NY-ESO-1₁₅₇ peptide. In order to examine the crossreactivity of the 3M4E5LH CAR and the 3M4E5H-L1 CAR to other peptides, human proteins that include a peptide that is capable of binding to HLA-A*02:01 and that may crossreact with the 3M4E5LH CAR and the 3M4E5H-L1 CAR were predicted using an algorithm (netMHC4.0, http://www.cbs.dtu.dk/services/NetMHC/). The uniprotKB/swissprot was used as the protein database. Moreover, homologous peptides that are highly homologous to the NY-ESO-1₁₅₇ peptide were selected from the peptides in the predicted proteins using an algorithm (ScanProsit, https://prosite.expasy.org/scanprosite/). As a result, Lysophospholipase-like protein 1 (LYPLAL1) protein that included a peptide (Sequence ID No. 207: GLRMWIKQV) that may crossreact therewith was hit. Accordingly, the LYPLAL1 protein was expressed in K562 cells, and the crossreactivity of the 3M4E5LH CAR and the 3M4E5H-L1 CAR was examined.

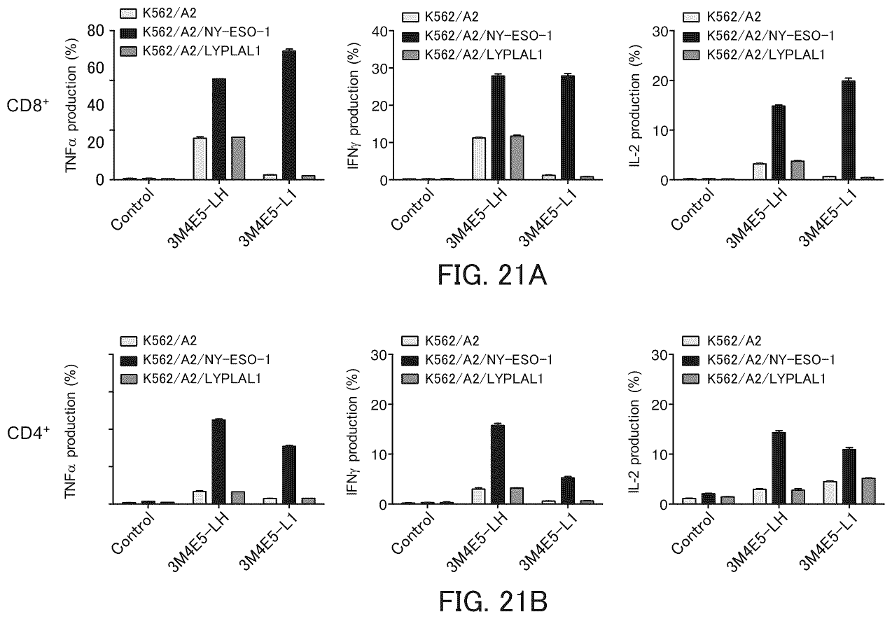

Retroviruses were used to introduce, into K562 cells (HLA and CD19 were not expressed), a nucleic acid coding for HLA-A*02:01 and a nucleic acid coding for LYPLAL1 (Sequence ID No. 208) or NY-ESO-1 (Sequence ID No. 209) to which ΔNGFR was added via the furin cleavage site, the spacer sequence, and the codon-optimized P2A sequence, and thus antigen-presenting cells (K562/A2/NY-ESO-1 or K562/A2/LYPLAL1) were obtained. Moreover, antigen-presenting cells (K562/A2) were prepared in the same manner, except that the nucleic acid coding for NY-ESO-1 or LYPLAL1 to which ΔNGFR was added was not introduced. The 3M4E5LH CAR-Ts and the 3M4E5H-L1 CAR-Ts were prepared in the same manner as in Example 6, and the CAR-T cells and the antigen-presenting cells were seeded in each well (96-well plate) such that the number of the CAR-T cells was 3×10⁵ and the number of the antigen-presenting cells was 5×10⁴, and then they were cultured together for 5 hours. An RPMI1640 culture medium containing FCS at a concentration of 10% was used as the culture solution for the coculture. After the above-mentioned coculture, the CAR-T cells were collected, fixed with paraformaldehyde, subjected to permeabilization, and stained with an APC-labeled anti-human IL-2 mAb (clone: MQ1-17H12), a PE-labeled anti-human TNFα mAb (clone: Mab11), a PC7-labeled anti-human IFN-γ antibody (clone: B27), an FITC-labeled anti-human CD4 mAb (clone: OKT4), a PC5-labeled anti-human CD8a mAb (clone: B9.11), and a V450-labeled anti-human NGFR mAb (clone: C40-1457). After the CAR-Ts were stained, the amounts of TNF-α, IFN-γ, and IL-2 produced by CD8-positive cells or CD4-positive cells in the CAR-Ts (NGFR-positive fraction) were measured using the flow cytometer. In a control 1, K562/A2 into which the NY-ESO-1 gene had not been introduced was used as the APCs. A control 2 was performed in the same manner, except that the expression vectors for expressing the hH-3M4E4L CAR library were not introduced. Then, the rates of an increase in cytokine production in the samples were calculated using the cytokine production amount in the control 2 as a standard. FIG. 21(A) and FIG. 21(B) show the results.

Nucleic Acid Molecule Coding for LYPLAL1
(Sequence ID No. 208)
5′-ATGGCGGCTGCATCTGGAAGCGTGCTGCAGAGATGTATCGTGTC
CCCAGCCGGCAGACATAGCGCCAGCCTGATTTTTCTGCACGGCAGCG
GCGATTCTGGCCAGGGACTGAGAATGTGGATCAAACAGGTGCTGAAC
CAGGACCTGACCTTCCAGCACATCAAGATCATCTACCCCACCGCTCC
ACCTCGGAGCTACACACCTATGAAGGGCGGCATCAGCAACGTTTGGT
TCGACCGGTTCAAGATCACCAACGACTGCCCCGAGCACCTGGAATCC
ATCGACGTGATGTGTCAGGTGCTCACCGACCTGATCGACGAGGAAGT
GAAGTCCGGCATCAAGAAGAACAGAATCCTGATCGGCGGCTTCAGCA
TGGGCGGCTGTATGGCCATTCACCTGGCCTACAGAAACCACCAGGAT
GTGGCTGGCGTGTTCGCCCTGAGCAGCTTTCTGAACAAAGCCAGCGC
CGTGTATCAGGCCCTGCAGAAATCTAACGGCGTGCTGCCTGAGCTGT
TCCAGTGTCATGGCACAGCCGATGAGCTGGTGCTGCACTCTTGGGCC
GAAGAGACAAATAGCATGCTGAAAAGCCTGGGCGTGACCACCAAGTT
CCACAGCTTCCCCAACGTGTACCACGAGCTGAGCAAGACCGAGCTGG
ACATCCTGAAACTGTGGATTCTGACCAAGCTGCCCGGCGAGATGGAA
AAGCAGAAG-3′
Nucleic Acid Molecule Coding for NY-ESO-1
(Sequence ID No. 209)
5′-ATGCAGGCCGAAGGCCGGGGCACAGGGGGTTCGACGGGCGATGC
TGATGGCCCAGGAGGCCCTGGCATTCCTGATGGCCCAGGGGGCAATG
CTGGCGGCCCAGGAGAGGCGGGTGCCACGGGCGGCAGAGGTCCCCGG
GGCGCAGGGGCAGCAAGGGCCTCGGGGCCGGGAGGAGGCGCCCCGCG
GGGTCCGCATGGCGGCGCGGCTTCAGGGCTGAATGGATGCTGCAGAT
GCGGGGCCAGGGGGCCGGAGAGCCGCCTGCTTGAGTTCTACCTCGCC
ATGCCTTTCGCGACACCCATGGAAGCAGAGCTGGCCCGCAGGAGCCT
GGCCCAGGATGCCCCACCGCTTCCCGTGCCAGGGGTGCTTCTGAAGG
AGTTCACTGTGTCCGGCAACATACTGACTATCCGACTGACTGCTGCA
GACCACCGCCAACTGCAGCTCTCCATCAGCTCCTGTCTCCAGCAGCT
TTCCCTGTTGATGTGGATCACGCAGTGCTTTCTGCCCGTGTTTTTGG
CTCAGCCTCCCTCAGGGCAGAGGCGC-3′

FIG. 21(A) and FIG. 21(B) show graphs indicating the ratios of cytokine-producing cells. FIG. 21(A) shows the results from CD8-positive T cells, and FIG. 21(B) shows the results from CD4-positive T cells. In FIG. 21(A) and FIG. 21(B), the horizontal axes of the graphs indicate the types of samples (CARs), and the vertical axes indicate the ratio of TNF-α-positive cells, IFN-γ-positive cells, or IL-2-positive cells. As shown in FIGS. 21(A) and 21(B), the cytokine production did not increase in the control 1 (Control). On the other hand, the T cells expressing the 3M4E5H-L1 CAR or 3M4E5LH CAR produced TNF-α, IFN-γ, and IL-2 in the positive control (K562/A2/NY-ESO-1). Moreover, as shown in FIGS. 21(A) and 21(B), when coculture with the antigen-presenting cell presenting LYPLAL1 (K562/A2/LYPLAL1) was performed, the ratio of cytokine-producing cells in the T cells expressing the 3M4E5LH CAR and 3M4E5H-L1 CAR was nearly equal to that in the case of the control 2 (K562/A2), which corresponds to the background, and no crossreactivity was observed. It was found from these results that the screening method of the present invention in which the CAR library of the present invention is used could control the crossreactivity of scFvs included in CARs.

### Example 8

It was confirmed that the antigenic specificity of CARs obtained using the screening method of the present invention in which the CAR library of the present invention is used was changed by arranging the heavy-chain variable region and the light-chain variable region in the reverse order.

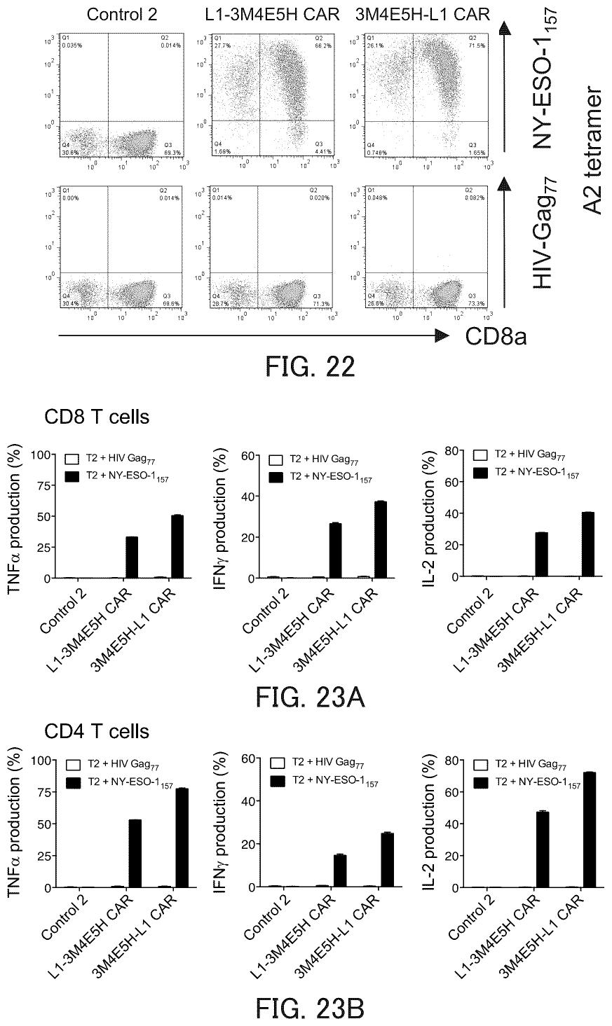

A culture supernatant containing GaLV-pseudotyped retroviruses was prepared in the same manner as in (3) of Example 1 above, except that the 3M4E5H-L1 expression vector or L1-3M4E5H expression vector was used instead of the expression vectors for expressing the hH-3M4E4L CAR library. Next, T cells were infected with the retroviruses in the same manner as in (3) of Example 1 above, except that the obtained GaLV-pseudotyped retroviruses were used. Thus, the 3M4E5H-L1 expression vector or L1-3M4E5H expression vector was introduced into the T cells. The infection with the retroviruses and the introduction of the 3M4E5H-L1 expression vector or L1-3M4E5H expression vector were performed in the same manner five more times (six times in total). The resulting T cells were stained with the PE-labeled A2/NY-ESO-1₁₅₇ tetramer at a concentration of 20 μg/mL. After the above-mentioned staining, the T cells were further stained with a PC5-labeled anti-human CD8a mAb (clone: B9.11), an FITC-labeled anti-human CD4 mAb (clone: OKT4), and a V450-labeled anti-human NGFR mAb (clone: C40-1457). The concentrations of these antibodies were about 10 μg/mL. Then, it was confirmed using the flow cytometer if the T cells that had been subjected to tetramer staining included T cells expressing CARs capable of binding to the A2/NY-ESO-1₁₅₇. A control 1 was performed in the same manner, except that the A2/HIV-Gag₇₇ tetramer was used instead of the A2/NY-ESO-1₁₅₇ tetramer. A control 2 was performed in the same manner, except that the expression vectors for expressing the hH-3M4E4L CAR library were not introduced. FIG. 22 shows the results obtained when gating of FSC⁺-SSC⁺-NGFR⁺ cells was performed.

FIG. 22 shows dot plots showing the results of flow cytometry. In FIG. 22, the horizontal axes indicate the average fluorescence intensity of CD8a, and the vertical axes indicate the average fluorescence intensity of the A2 tetramer. As shown in FIG. 22, in the control 2, T cells capable of binding to the A2/NY-ESO-1₁₅₇ or A2/HIV-Gag₇₇ were not present. Moreover, in the case of the CAR-Ts expressing the L1-3M4E5H or 3M4E5H-L1, the CD8⁺-A2 tetramer⁺ fraction was present when A2/NY-ESO-1₁₅₇ tetramer staining was performed, and the CD8⁺-A2 tetramer⁺ fraction was not present when A2/HIV-Gag₇₇ tetramer staining was performed. Furthermore, when the CAR-Ts expressing the L1-3M4E5H and the CAR-Ts expressing the 3M4E5H-L1 included in the CD8⁻- or CD8⁺-A2 tetramer⁺ fraction were compared, the 3M4E5H-L1 CAR-Ts bound more tightly to the A2/NY-ESO-1₁₅₇ than the L1-3M4E5H CAR-Ts did in both the CD8-A2 tetramer⁺ fraction and the CD8⁺-A2 tetramer⁺ fraction, that is, the antigenic specificity of the 3M4E5H-L1 CAR-Ts was different from that of the L1-3M4E5H CAR-Ts.

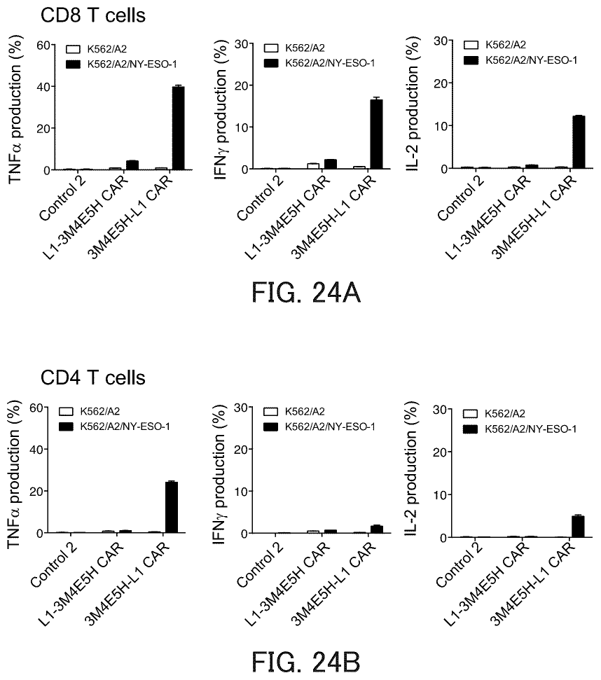

Next, it was examined if the T cells that had been infected with the retroviruses bound to the target antigen, and were thus activated to produce cytokines. Specifically, after the infection of T cells (CAR-Ts) with the retroviruses were repeated six times, the T cells, and T2 cells or APCs prepared in (4) of Example 1 above were seeded in each well (96-well plate) such that the number of the T cells was 3×10⁵ and the number of the T2 cells or APCs was 5×10⁴, and then they were cultured together for 5 hours. Before use, the T2 cells (HLA-A*02:01-expressing cells) and APCs were pulsed with the NY-ESO-1₁₅₇ peptide at a concentration of 10 μg/mL (T2+NY-ESO-1₁₅₇ and K562/A2/NY-ESO-1). An RPMI1640 culture medium containing FCS at a concentration of 10% was used as the culture solution for the coculture. After the above-mentioned coculture, the T cells were collected, fixed with paraformaldehyde, subjected to permeabilization, and stained with an APC-labeled anti-human IL-2 mAb (clone: MQ1-17H12), a PE-labeled anti-human TNFα mAb (clone: Mab11), a PC7-labeled anti-human IFN-γ antibody (clone: B27), an FITC-labeled anti-human CD4 mAb (clone: OKT4), a PC5-labeled anti-human CD8a mAb (clone: B9.11), and a V450-labeled anti-human NGFR mAb (clone: C40-1457). After the CAR-Ts were stained, the amounts of TNF-α, IFN-γ, and IL-2 produced by CD8-positive cells or CD4-positive cells in the CAR-Ts (NGFR-positive fraction) were measured using the flow cytometer. When the T2 cells were used, a control 1 was performed in the same manner, except that the HIV-Gag₇₇ peptide was used instead of the NY-ESO-1₁₅₇ peptide. When the APCs (K562/A2/NY-ESO-1) were used, a control 1 was performed in the same manner, except that K562/A2 into which the NY-ESO-1 gene had not been introduced was used. A control 2 was performed in the same manner, except that the expression vectors for expressing the hH-3M4E4L CAR library were not introduced. Then, the rates of an increase in cytokine production in the samples were calculated using the cytokine production amount in the control 2 as a standard. FIGS. 23 and 24 show the results.

FIG. 23(A) and FIG. 23(B) show the results obtained when the T2 cells were used. FIG. 23(A) and FIG. 23(B) show graphs indicating changes in a cytokine production amount. FIG. 23(A) shows the results from CD8-positive T cells, and FIG. 23(B) shows the results from CD4-positive T cells. In FIG. 23(A) and FIG. 23(B), the horizontal axes indicate the types of samples (CARs), and the vertical axes indicate the rate of an increase in cytokine production. As shown in FIG. 23(A) and FIG. 23(B), the cytokine production did not increase in the control 2. On the other hand, when the L1-3M4E5H CAR-Ts or 3M4E5H-L1 CAR-Ts were stimulated with the T2+NY-ESO-1₁₅₇, it could be confirmed that the cytokine production amount increased compared with the case of the stimulation with the T2+HIV Gag₇₇, and the cytokines bound to the A2/NY-ESO-1₁₅₇ to activate the CAR-Ts. Moreover, when the stimulation with T2+NY-ESO-1₁₅₇ was performed, the 3M4E5H-L1 CAR-Ts produced a larger amount of cytokines than the L1-3M4E5H CAR-Ts did.

FIG. 24(A) and FIG. 24(B) show the results obtained when the APCs were used. FIG. 24(A) and FIG. 24(B) show graphs indicating changes in a cytokine production amount. FIG. 24(A) shows the results from CD8-positive T cells, and FIG. 24(B) shows the results from CD4-positive T cells. In FIG. 24(A) and FIG. 24(B), the horizontal axes indicate the types of samples (CARs), and the vertical axes indicate the rate of an increase in cytokine production. As shown in FIG. 24(A) and FIG. 24(B), the cytokine production did not increase in the control 2. On the other hand, when the L1-3M4E5H CAR-Ts or 3M4E5H-L1 CAR-Ts were stimulated with the K562/A2/NY-ESO-1, it could be confirmed that the cytokine production amount increased compared with the case of the stimulation with the K562/A2, and the cytokines bound to the K562/A2/NY-ESO-1 to activate the CAR-Ts. Moreover, when the stimulation with the K562/A2/NY-ESO-1 was performed, the 3M4E5H-L1 CAR-Ts produced a larger amount of cytokines than the L1-3M4E5H CAR-Ts did. It was found that the antigenic specificity of CARs obtained using the screening method of the present invention in which the CAR library of the present invention is used was changed by arranging the heavy-chain variable regions and the light-chain variable regions in the reverse order. Moreover, it was found that the specificity, for a target antigen, of CARs obtained using the screening method of the present invention in which the CAR library of the present invention is used could be possibly improved by arranging the heavy-chain variable regions and the light-chain variable regions in the reverse order.

### Example 9

It was confirmed that the first screening method of the present invention could be used to screen scFvs that recognize different sites of a target antigen.

First, the CARs expressed by the second candidate CAR-Ts and the CARs expressed by the first candidate CAR-Ts were identified in the same manner as in (9) to (12) of Example 1 above and (1) to (3) of Example 2 above, and thus new CARs (L52-3M4E5H CAR and K156-H1 CAR) were obtained. The underlined sequences in the following amino acid sequences and base sequences correspond to the amino acid sequences or base sequences of the CDRL1, the CDRL2, and the CDRL3 of L52 or L156.

L52-3M4E5H CAR
(Sequence ID No. 210)
QSVLTQPPSVSGAPGQRVTISCTGSSSNLGAGYDVHWYQQLPGTAPK
LLIYGSTTRPSGIPDRFSGSKSGTSATLGITGLQTGDEADYYCGTWD
SSLSAGVFGGGTQLTVLGSTSGSGKPGSGEGSTKGEVQLLESGGGLV
QPGGSLRLSCAASGFTFSTYQMSWVRQAPGKGLEWVSGIVSSGGSTA
YADSVKGRFTISRDNSKNTLYLQMNSLRAEDTAVYYCAGELLPYYGM
DVWGQGTTVTVSSAAAIEVMYPPPYLDNEKSNGTIIHVKGKHLCPSP
LFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQN
QLYNELNLGRREEYDVLDKRRGRDPEMGGKPRRKNPQEGLYNELQKD
KMAEAYSEIGMKGERRRGKGHDGLYQGLSTATKDTYDALHMQALPPR
Polynucleotide Coding for L52-3M4E5H CAR
(Sequence ID No. 211)
5′-CAGTCTGTGCTGACGCAGCCGCCCTCAGTGTCTGGGGCCCCAGG
GCAGAGGGTCACCATCTCCTGCACTGGGAGCAGCTCCAACCTCGGGG
CAGGTTATGATGTACACTGGTACCAGCAGCTTCCAGGAACAGCCCCC
AAACTCCTCATCTATGGTAGCACCACTCGGCCCTCAGGGATTCCTGA
CCGATTCTCTGGCTCCAAGTCTGGCACGTCAGCCACCCTGGGCATCA
CCGGACTCCAGACTGGGGACGAGGCCGATTATTACTGCGGAACATGG
GATAGCAGCCTGAGTGCTGGGGTGTTCGGCGGAGGCACCCAGCTGAC
CGTCCTCGGCTCTACAAGCGGCTCTGGCAAGCCTGGATCTGGCGAGG
GAAGCACCAAGGGCGAAGTGCAGCTGCTGGAATCTGGCGGCGGACTG
GTGCAGCCTGGCGGATCTCTGAGACTGAGCTGTGCCGCCAGCGGCTT
CACCTTCAGCACCTACCAGATGAGCTGGGTGCGCCAGGCCCCTGGCA
AAGGACTGGAATGGGTGTCCGGCATCGTGTCCAGCGGCGGCTCTACA
GCCTACGCCGATAGCGTGAAGGGCCGGTTCACCATCAGCCGGGACAA
CAGCAAGAACACCCTGTACCTGCAGATGAACAGCCTGAGAGCCGAGG
ACACCGCCGTGTACTATTGTGCCGGGGAGCTGCTGCCCTACTACGGC
ATGGATGTGTGGGGCCAGGGCACCACCGTGACAGTGTCCTCAGCGGC
CGCAATCGAAGTGATGTACCCCCCTCCCTACCTGGACAACGAGAAGT
CCAACGGCACCATTATCCACGTGAAGGGAAAGCACCTGTGCCCCAGC
CCTCTGTTCCCTGGCCCTAGCAAGCCTTTCTGGGTGCTGGTGGTCGT
GGGCGGAGTGCTGGCCTGTTATAGCCTGCTCGTGACCGTGGCCTTCA
TCATCTTTTGGGTGCGCAGCAAGCGGAGCCGGCTGCTGCACAGCGAC
TACATGAACATGACCCCCAGACGGCCCGGACCCACCAGAAAGCACTA
CCAGCCTTACGCCCCTCCCAGAGACTTCGCCGCCTACAGATCTCGAG
TGAAGTTCAGCAGAAGCGCCGACGCCCCTGCCTATCAGCAGGGCCAG
AACCAGCTGTACAACGAGCTGAACCTGGGCAGACGGGAAGAGTACGA
CGTGCTGGACAAGCGGAGAGGCAGGGACCCTGAGATGGGCGGCAAGC
CCAGAAGAAAGAACCCCCAGGAAGGCCTGTATAACGAACTGCAGAAA
GACAAGATGGCCGAGGCCTACAGCGAGATCGGCATGAAGGGCGAGCG
GAGAAGAGGCAAGGGCCACGATGGCCTGTACCAGGGCCTGAGCACCG
CCACCAAGGACACCTATGACGCCCTGCACATGCAGGCCCTGCCCCCC
AGA-3′
K156-H1 CAR
(Sequence ID No. 212)
EIVLTQSPATLSVSPGERATLSCRASQSVSSNLAWYQQKPGQAPRLL
IYGASTRATGIPARFSGSGSGTEFTLTISRLEPEDFAVYYCQQYNNW
PPKFTFGPGTKVDIRGSTSGSGKPGSGEGSTKGQVQLQESGPGLVKP
SDTLSLTCLVSGGSISSNYWSWIRQAPGKGLEWIGHVSYSGSTNYNP
SLKSRVTISVDTSKNQFSLKLSSVTAADTAVYYCARESYYYYGMDVW
GQGTTVTVSSAAAIEVMYPPPYLDNEKSNGTIIHVKGKHLCPSPLFP
GPSKPFWVLVVVGGVLACYSLLVTVAFIIFWVRSKRSRLLHSDYMNM
TPRRPGPTRKHYQPYAPPRDFAAYRSRVKFSRSADAPAYQQGQNQLY
NELNLGRREEYDVLDKRRGRDPEMGGKPRRKNPQEGLYNELQKDKMA
EAYSEIGMKGERRRGKGHDGLYQGLSTATKDTYDALHMQALPPR
Polynucleotide Coding for K156-H1 CAR
(Sequence ID No. 213)
5′-GAAATTGTGTTGACGCAGTCTCCAGCCACCCTGTCTGTGTCTCC
AGGGGAAAGAGCCACCCTCTCCTGCAGGGCCAGTCAGAGTGTTAGCA
GCAACTTAGCCTGGTACCAGCAGAAACCTGGCCAGGCTCCCAGGCTC
CTCATCTATGGTGCATCCACCAGGGCCACTGGTATCCCAGCCAGGTT
CAGTGGCAGTGGGTCTGGGACAGAGTTCACTCTCACCATCAGCAGAC
TGGAGCCTGAAGATTTTGCAGTGTATTACTGTCAGCAGTATAATAAC
TGGCCTCCGAAATTCACTTTCGGCCCTGGGACCAAAGTGGATATCAG
AGGCTCTACAAGCGGCTCTGGCAAGCCTGGATCTGGCGAGGGAAGCA
CCAAGGGCCAGGTGCAGCTGCAGGAGTCGGGCCCAGGACTGGTGAAG
CCTTCGGATACCCTGTCCCTCACCTGTCTTGTCTCTGGTGGCTCCAT
CAGTAGTAATTACTGGAGCTGGATCCGGCAGGCCCCAGGGAAGGGAC
TGGAGTGGATTGGACATGTCTCCTACAGTGGGAGCACCAACTACAAC
CCCTCCCTCAAGAGTCGAGTTACCATATCAGTAGACACGTCTAAGAA
CCAGTTCTCCCTGAAGCTGAGCTCTGTGACTGCCGCGGACACGGCCG
TGTATTACTGTGCGAGAGAGTCCTACTACTACTACGGTATGGACGTC
TGGGGCCAAGGGACCACGGTCACCGTCTCCTCAGCGGCCGCAATCGA
AGTGATGTACCCCCCTCCCTACCTGGACAACGAGAAGTCCAACGGCA
CCATTATCCACGTGAAGGGAAAGCACCTGTGCCCCAGCCCTCTGTTC
CCTGGCCCTAGCAAGCCTTTCTGGGTGCTGGTGGTCGTGGGCGGAGT
GCTGGCCTGTTATAGCCTGCTCGTGACCGTGGCCTTCATCATCTTTT
GGGTGCGCAGCAAGCGGAGCCGGCTGCTGCACAGCGACTACATGAAC
ATGACCCCCAGACGGCCCGGACCCACCAGAAAGCACTACCAGCCTTA
CGCCCCTCCCAGAGACTTCGCCGCCTACAGATCTCGAGTGAAGTTCA
GCAGAAGCGCCGACGCCCCTGCCTATCAGCAGGGCCAGAACCAGCTG
TACAACGAGCTGAACCTGGGCAGACGGGAAGAGTACGACGTGCTGGA
CAAGCGGAGAGGCAGGGACCCTGAGATGGGCGGCAAGCCCAGAAGAA
AGAACCCCCAGGAAGGCCTGTATAACGAACTGCAGAAAGACAAGATG
GCCGAGGCCTACAGCGAGATCGGCATGAAGGGCGAGCGGAGAAGAGG
CAAGGGCCACGATGGCCTGTACCAGGGCCTGAGCACCGCCACCAAGG
ACACCTATGACGCCCTGCACATGCAGGCCCTGCCCCCCAGA-3′

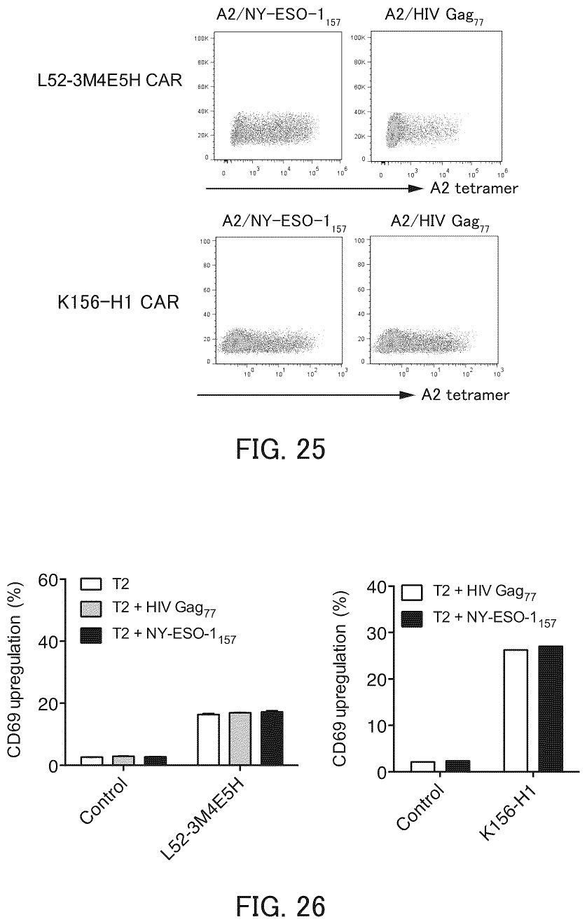

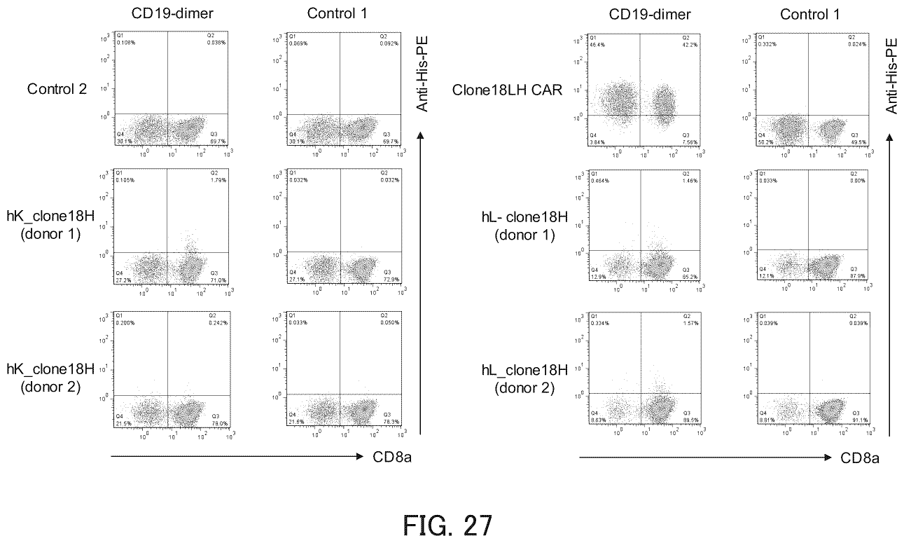

The ability to bind to the A2/NY-ESO-1₁₅₇ tetramer and the A2/HIV-Gag₇₇ tetramer, and the rate of an increase in CD69 expression were analyzed by flow cytometry in the same manner as in (6) and (8) of Example 1 above, except that a CAR expression vector was used into which a polynucleotide coding for the L52-3M4E5H CAR or K156-H1 CAR had been introduced instead of the polynucleotides coding for scFvs included in the expression vectors for expressing the hH-3M4E4L CAR library. A control 1 was performed in the same manner, except that the rate of an increase in CD69 expression was analyzed without adding the CAR-Ts. A control 2 was performed in the same manner, except that pulsing with the NY-ESO-1₁₅₇ peptide or HIV Gag₇₇₋₈₅ peptide was not performed. FIGS. 25 and 26 show the results.

FIG. 25 shows dot plots showing the results of flow cytometry. In FIG. 25, the horizontal axes indicate the average fluorescence intensity resulting from A2/NY-ESO-1₁₅₇ tetramer staining or A2/HIV-Gag₇₇ tetramer staining, and the vertical axes indicate the average side scatter (SSC) intensity. As shown in FIG. 25, the L52-3M4E5H CAR-Ts and the K156-H1 CAR-Ts included fractions that had been stained with both the A2/NY-ESO-1₁₅₇ tetramer and the A2/HIV-Gag₇₇ tetramer. That is, the L52-3M4E5H CAR and the K156-H1 CAR bound to the A2/NY-ESO-1₁₅₇ and the A2/HIV-Gag₇₇. It is inferred that this is because the scFvs of the L52-3M4E5H CAR and the K156-H1 CAR recognize and bind to sites closer to an HLA molecule in a complex of the peptide and the HLA molecule compared with the scFv and the like of the L1-3M4E5H CAR.

FIG. 26 shows graphs indicating changes in a CD69 expression level obtained when the peptide concentration was 10 μg/mL. In FIG. 26, the horizontal axes indicate the types of samples, and the vertical axes indicate the rate of an increase in CD69 expression. As shown in FIG. 26, the CD69 expression did not increase in the control 1. On the other hand, when the L52-3M4E5H CAR-Ts and the K156-H1 CAR-Ts were stimulated with the antigen-presenting cells (control 2, T2) that had not been pulsed with the peptide and the antigen-presenting cells that had been pulsed with the A2/NY-ESO-1₁₅₇ (T2+NY-ESO-1₁₅₇) or the A2/HIV-Gag₇₇ (T2+HIV Gag₅₇), the CD69 expression increased. Since CD69 was also expressed in the control 2, it was corroborated that the scFvs of the L52-3M4E5H CAR and the K156-H1 CAR recognize and bind to sites closer to an HLA molecule in a complex of the peptide and the HLA molecule compared with the scFv and the like of the L1-3M4E5H CAR.

It was found from these results that the first screening method of the present invention could be used to screen scFvs that recognize different sites of a target antigen. That is, it was found that scFvs having the ability to bind to different sites of a target antigen could be screened.

### Example 10

It was confirmed that the screening method of the present invention in which the CAR library of the present invention is used could be used to screen scFvs capable of binding to CD19.

### (1) Preparation of Clone 18 CARs

A clone 18 scFv or a nucleic acid coding for the clone 18 scFv was prepared in the same manner as in (1) of Example 3 above, except that the heavy-chain variable region and the light-chain variable region of a human CD19-specific antibody (clone18 (see WO 2016/033570)) were used instead of the heavy-chain variable region and the light-chain variable region of the human CD19-specific antibody FMC63. The nucleic acid was introduced into a pMX expression vector. It should be noted that the amino acid sequences that correspond to the heavy-chain variable region and the light-chain variable region in the scFv were underlined.

clone18LH scFv
(Sequence ID No. 214)
QSALTQPRSVSGFPGQSVTISCTGTTSDDVSWYQQHPGKAPQLMLYD
VSKRPSGVPHRFSGSRSGRAASLIISGLQTEDEADYFCCSYAGRYNS
VLFGGGTKLTVLGSTSGSGKPGSGEGSTKGEVQLVESGGGLVQPGRS
LRLSCAASGFTFDDYAMHWVRQAPGKGLEWVSGISWNSGRIGYADSV
KGRFTISRDNAKNSLFLQMNSLRAEDTAVYYCARDQGYHYYDSAEHA
FDIWGQGTVVTVSS
Nucleic Acid Coding for clone 18LH scFv
(Sequence ID No. 215)
5′-CAATCTGCTCTGACACAGCCTAGAAGCGTGTCCGGCTTTCCTGG
CCAGAGCGTGACCATCAGCTGTACCGGCACCACCTCCGATGACGTGT
CCTGGTATCAGCAGCATCCTGGCAAAGCCCCTCAGCTGATGCTGTAC
GACGTGTCCAAAAGACCTAGCGGCGTGCCCCACAGATTCAGCGGATC
TAGAAGTGGCAGAGCCGCCAGCCTGATCATCTCTGGACTGCAGACAG
AGGACGAGGCCGACTACTTCTGCTGTAGCTACGCCGGCAGATACAAC
AGCGTGCTGTTTGGCGGCGGAACAAAGCTGACAGTGCTGGGCTCTAC
AAGCGGCTCTGGCAAGCCTGGATCTGGCGAGGGAAGCACCAAGGGCG
AAGTGCAGCTTGTGGAATCTGGCGGAGGACTGGTGCAGCCTGGAAGA
AGCCTGAGACTGTCTTGTGCCGCCAGCGGCTTCACCTTCGACGATTA
TGCCATGCACTGGGTCCGACAGGCCCCTGGAAAAGGCCTTGAATGGG
TGTCCGGCATCTCTTGGAACAGCGGCAGAATCGGCTACGCCGACTCT
GTGAAGGGCAGATTCACCATCAGCCGGGACAACGCCAAGAACAGCCT
GTTCCTGCAGATGAACTCCCTGAGAGCCGAGGACACCGCCGTGTACT
ACTGTGCCAGAGATCAGGGCTACCACTACTACGACTCTGCCGAGCAC
GCCTTCGATATCTGGGGCCAGGGAACAGTGGTCACCGTTAGTTCT-3′

### (2) Preparation of First CAR Library

An hK-clone18H scFv library and an hL-clone18H scFv library were prepared by substituting the nucleic acid coding for the light-chain variable region on the 5′ side of the nucleic acid coding for the clone18LH scFv described in (1) of Example 10 above with a library that included nucleic acids of the light-chain variable regions derived from human peripheral blood B cells. Then, the hK-clone18H scFv library and the hL-clone18H scFv library were prepared in the same manner as in (2) of Example 1 above, except that the hK-clone18H scFv library and the hL-clone18H scFv library were used instead of the nucleic acid coding for the 3M4E5LH scFv. Next, expression vectors for expressing an hK-clone18H CAR library and an hL-clone18H CAR library were prepared by introducing, into pMX expression vectors, the hK-clone18H scFv library and the hL-clone18H scFv library such that a nucleic acid coding for the partial region, the transmembrane domain, and the intracellular signaling domain of human CD28 described in (1) of Example 1 and a nucleic acid coding for the intracellular signaling domain of human CD32 were arranged in the stated order on the 3′ end side of the library. It should be noted that, regarding the heterogeneity of the light-chain variable regions in the hK-clone18H CAR library and the hL-clone18H CARs, it was confirmed through restriction enzyme mapping and sequencing of the regions using the Sanger's method that the number of types of light-chain variable regions was about 1×10⁶.

### (3) Preparation of First CAR Library-Expressing T Cells (First Candidate CAR-Ts)

The hK-clone18H CAR library and the hL-clone18H CAR library were introduced into T cells in the same manner as in (3) of Example 1 above, except that the expression vectors for expressing the hK-clone18H CAR library and the hL-clone18H CAR library were used instead of the expression vectors for expressing the hH-3M4E4L CAR library. Then, the resulting T cells were purified and used as first candidate CAR-Ts.

### (4) Preparation of Target Antigen-Expressing Cells

In the same manner as in (4) of Example 3 above, retroviruses were used to introduce the nucleic acid coding for human CD19 into K562 cells, and thus antigen-presenting cells (APCs-CD19) were obtained.

### (5) First Screening Method

First candidate CAR-Ts expressing hK-clone18H CARs or hL-clone18H CARs capable of binding to human CD19 was enriched in the same manner as in (5) of Example 3 above, except that the first candidate CAR-Ts described in (3) of Example 10 above and the APCs-CD19 prepared in (4) of Example 10 above were used.

Next, after the enrichment of the first candidate CAR-Ts, the first candidate CAR-Ts were collected and stained with a PE-labeled CD19 dimer at a concentration of 40 μg/mL. The PE-labeled CD19 dimer was prepared using a soluble CD19, which is human CD19 that includes a His-tag added to the C terminus of the extracellular domain thereof via an SGSG linker. Specifically, the PE-labeled CD19 dimer was prepared by mixing the soluble CD19 and a PE-labeled anti-His mAb (clone: GG11-8F3.5.1) such that the molar ratio therebetween was 2:1. After the above-mentioned staining, the T cells were further stained with a PC5-labeled anti-human CD8a mAb (clone: B9.11), an FITC-labeled anti-human CD4 mAb (clone: OKT4), and a V450-labeled anti-human NGFR mAb (clone: C40-1457). Then, regarding the first candidate CAR-Ts that had been subjected to dimer staining, it was confirmed using the flow cytometer if first candidate CAR-Ts expressing CARs capable of binding to CD19 were present. A positive control was performed in the same manner, except that an expression vector that included a nucleic acid coding for the clone18LH CAR was used instead of the expression vectors for expressing the hK-clone18H CAR library and the hL-clone18H CAR library. A control 1 was performed in the same manner, except that CD19 dimer staining was not performed. A control 2 was performed in the same manner, except that the expression vectors for expressing the hK-clone18H CAR library and the hL-clone18H CAR library were not introduced. The same test was performed using a library that included nucleic acids of light-chain variable regions derived from human peripheral blood B cells obtained from another healthy subject (donor 2). FIG. 27 shows the results obtained when gating of FSC⁺-SSC⁺-NGFR⁺ cells was performed.

FIG. 27 shows dot plots showing the results of flow cytometry. In FIG. 27, the horizontal axes indicate the average fluorescence intensity of CD8a, and the vertical axes indicate the average fluorescence intensity of the CD19 dimer. As shown in FIG. 27, the T cells (control 2) into which the expression vectors for expressing the hK-clone18H CAR library and the hL-clone18H CAR library had not been introduced did not include T cells capable of binding to the CD19 dimer. Moreover, it could be confirmed that the CAR-Ts (clone18LH, positive control) expressing the clone18LH CAR were capable of binding to the CD19 dimer because a CD19 dimer⁺ fraction (the upper right fraction in each dot plot) was present when CD19 dimer staining was performed, and the CD19 dimer⁺ fraction was not present when CD19 dimer staining was not performed. Furthermore, in all the groups of the first candidate CAR-Ts into which the expression vectors for expressing the hK-clone18H CAR library and the hL-clone18H CAR library had been introduced, the CD19 dimer⁺ fraction was present when CD19 dimer staining was performed, and the CD19 dimer⁺ fraction was not present when CD19 dimer staining was not performed. As a result, it was found that the first candidate CAR-Ts included first candidate CAR-Ts expressing CARs capable of binding to the CD19 dimer. It was found from these results that the first screening method of the present invention could be used to screen scFvs capable of binding to a target antigen even when different antibodies were used.

Next, first candidate CAR-Ts expressing CARs capable of binding to CD19 were selected, and the CARs expressed by the first candidate CAR-Ts were identified. Specifically, the first candidate CAR-Ts were stained in the same manner as in (5) of Example 10 above. Then, cell sorting was performed on the first candidate CAR-Ts using a flow cytometer (FACSAria (registered trademark), manufactured by Becton Dickinson), and thus an NGFR⁺-CD8⁺-CD19 dimer⁺ fraction or NGFR⁺-CD4⁺-CD19 dimer⁺ fraction was collected as first candidate CAR-Ts expressing CARs capable of binding to CD19. The total RNA was extracted from the obtained first candidate CAR-Ts using a total RNA extraction reagent (TRIzol (registered trademark), manufactured by Ambion). cDNA was synthesized from the total RNA using a reverse transcriptase (Superscript (registered trademark) III, manufactured by Thermo Fisher Scientific).

PCR in which the obtained cDNA and a mixture of the forward primer 1 (Sequence ID No. 199) and the reverse primer 1 (Sequence ID No. 200) were used was performed to amplify nucleic acids coding for scFvs. A cloning kit (Gibson Assembly Master Mix, manufactured by New England Biolab) was used to introduce the obtained nucleic acids coding for scFvs into pMX/CAR expression vectors (pMX/scFv expression vectors) such that the nucleic acid coding for an scFv bound to the 5′ end of the partial region of human CD28. The pMX/CAR expression vectors were prepared by linking a nucleic acid (Sequence ID No. 169) coding for the partial region, the transmembrane domain, and the intracellular signaling domain of human CD28 and a nucleic acid (Sequence ID No. 171) coding for the intracellular signaling domain of human CD3 (such that these nucleic acids were arranged in the stated order from the 5′ end side and introducing the thus obtained product into the pMX expression vector. Then, sequencing of the obtained pMX/scFv expression vectors was performed using the Sanger's method, and thus base sequences coding for CARs (a polynucleotide (Sequence ID No. 298) coding for an L4-18H CAR, a polynucleotide (Sequence ID No. 299) coding for an L7-18H CAR, a polynucleotide (Sequence ID No. 300) coding for an L9-18H CAR, a polynucleotide (Sequence ID No. 301) coding for an L13-18H CAR, a polynucleotide (Sequence ID No. 302) coding for an L14-18H CAR, a polynucleotide (Sequence ID No. 303) coding for an L16-18H CAR, a polynucleotide (Sequence ID No. 304) coding for an L17-18H CAR, a polynucleotide (Sequence ID No. 305) coding for an L22-18H CAR, a polynucleotide (Sequence ID No. 306) coding for a K4-18H CAR, a polynucleotide (Sequence ID No. 307) coding for a K5-18H CAR, a polynucleotide (Sequence ID No. 308) coding for a K6-18H CAR, a polynucleotide (Sequence ID No. 309) coding for a K9-18H CAR, and a polynucleotide (Sequence ID No. 310) coding for a K10-18H CAR) were identified. Moreover, the amino acid sequences of the scFvs (L4-18H (Sequence ID No. 272), L7-18H (Sequence ID No. 273), L9-18H (Sequence ID No. 274), L13-18H (Sequence ID No. 275), L14-18H (Sequence ID No. 276), L16-18H (Sequence ID No. 277), L17-18H (Sequence ID No. 278), L22-18H (Sequence ID No. 279), K4-18H (Sequence ID No. 280), K5-18H (Sequence ID No. 281), K6-18H (Sequence ID No. 282), K9-18H (Sequence ID No. 283), and K10-18H (Sequence ID No. 284)) were identified based on the above-mentioned base sequences. Then, the CDRL1, CDRL2, and the CDRL3 in the new light-chain variable regions (LA) to (LM), which are shown in Table 3B above, were identified based on the amino acid sequences of the scFvs. Furthermore, the pMX/scFv expression vectors were used to prepare expression vectors that included the polynucleotides coding for the CARs (a pMX/L4-18H expression vector, a pMX/L7-18H expression vector, a pMX/L9-18H expression vector, a pMX/L13-18H expression vector, a pMX/L14-18H expression vector, a pMX/L16-18H expression vector, a pMX/L17-18H expression vector, a pMX/L22-18H expression vector, a pMX/K4-18H expression vector, a pMX/K5-18H expression vector, a pMX/K6-18H expression vector, a pMX/K9-18H expression vector, and a pMX/K10-18H expression vector).

(6) Analysis of First Candidate scFvs

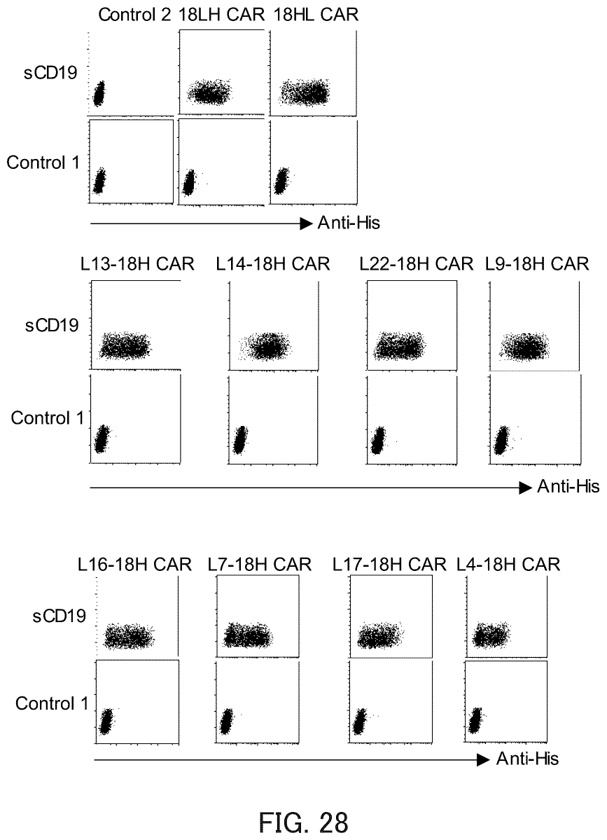

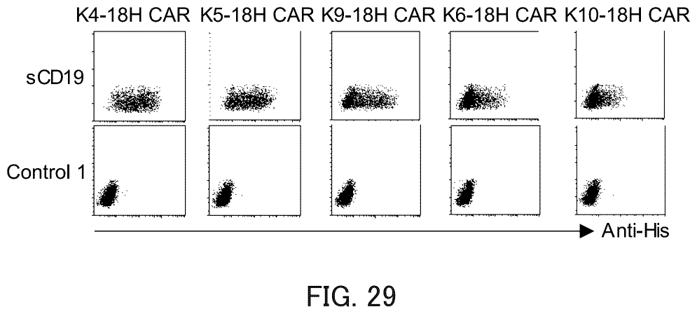

A culture supernatant containing GaLV-pseudotyped retroviruses was prepared in the same manner as in (3) of Example 10 above, except that the expression vectors that included the polynucleotides coding for the CARs were used instead of the expression vectors for expressing the hK-clone18H CAR library and the hL-clone18H CAR library. Next, the expression vectors that included the polynucleotides coding for the CARs were introduced into Jurkat 76 cells by adding the above-mentioned culture supernatant containing GaLV-pseudotyped retroviruses, and a CAR (an L4-18H CAR, an L7-18H CAR, an L9-18H CAR, an L13-18H CAR, an L14-18H CAR, an L16-18H CAR, an L17-18H CAR, an L22-18H CAR, a K4-18H CAR, a K5-18H CAR, a K6-18H CAR, a K9-18H CAR, or a K10-18H CAR) that included the L4-18H, L7-18H, L9-18H, L13-18H, L14-18H, L16-18H, L17-18H, L22-18H, K4-18H, K5-18H, K6-18H, K9-18H, or K10-18H as an scFv was expressed. The Jurkat 76 cells that had expressed the CARs were reacted with the PE-labeled anti-human NGFR mAb. After the above-mentioned reaction, Jurkat 76 cells (L4-18H CAR-Ts, L7-18H CAR-Ts, L9-18H CAR-Ts, L13-18H CAR-Ts, L14-18H CAR-Ts, L16-18H CAR-Ts, L17-18H CAR-Ts, L22-18H CAR-Ts, K4-18H CAR-Ts, K5-18H CAR-Ts, K6-18H CAR-Ts, K9-18H CAR-Ts, or K10-18H CAR-Ts) expressing the CARs were purified using the anti-PE microbeads. The Jurkat 76 cells expressing the CARs were stained in the same manner as in (5) of Example 10 above. The stained Jurkat 76 cells expressing the CARs were analyzed by flow cytometry. A positive control was performed in the same manner, except that an expression vector that included a nucleic acid coding for a tagged clone18LH CAR or a tagged clone18HL CAR was used instead of the expression vectors that included the polynucleotides coding for the CARs. A control 1 was performed in the same manner, except that CD19 dimer staining was not performed. A control 2 was performed in the same manner, except that the expression vectors that included the polynucleotides coding for the CARs were not introduced. FIGS. 28 and 29 show the results obtained when gating of FSC⁺-SSC⁺-NGFR⁺ cells was performed.

FIGS. 28 and 29 show dot plots showing the results of flow cytometry. In FIGS. 28 and 29, the horizontal axes indicate the average fluorescence intensity resulting from CD19 dimer staining, and the vertical axes indicate the average side scatter (SSC) intensity. As shown in FIGS. 28 and 29, the Jurkat 76 cells (control 2) into which the expression vectors that included the polynucleotides coding for the CARs had not been introduced did not include cells capable of binding to CD19. Moreover, it could be confirmed that the clone18LH CAR-Ts and the clone18HL CAR-Ts (clone18LH CAR or clone18HL CAR, positive control) bound to CD19 because substantially all the cells were present as CD19 dimer⁺ cells when CD19 dimer staining was performed, and substantially all the cells were present as CD19 dimer⁻ cells when CD19 dimer staining was performed. Furthermore, the L4-18H CAR-Ts, L7-18H CAR-Ts, L9-18H CAR-Ts, L13-18H CAR-Ts, L14-18H CAR-Ts, L16-18H CAR-Ts, L17-18H CAR-Ts, L22-18H CAR-Ts, K4-18H CAR-Ts, K5-18H CAR-Ts, K6-18H CAR-Ts, K9-18H CAR-Ts, or K10-18H CAR-Ts were present as CD19 dimer⁺ CAR-Ts when CD19 dimer staining was performed, and the CAR-Ts were present as CD19 dimer⁻ CAR-Ts when CD19 dimer staining was not performed. It was found from these results that the L4-18H CAR-Ts, L7-18H CAR-Ts, L9-18H CAR-Ts, L13-18H CAR-Ts, L14-18H CAR-Ts, L16-18H CAR-Ts, L17-18H CAR-Ts, L22-18H CAR-Ts, K4-18H CAR-Ts, K5-18H CAR-Ts, K6-18H CAR-Ts, K9-18H CAR-Ts, or K10-18H CAR-Ts were capable of binding to the CD19 dimer. That is, it was found that the first screening method of the present invention could be used to screen scFvs capable of binding to a target antigen.

(7) Functions of First Candidate scFvs

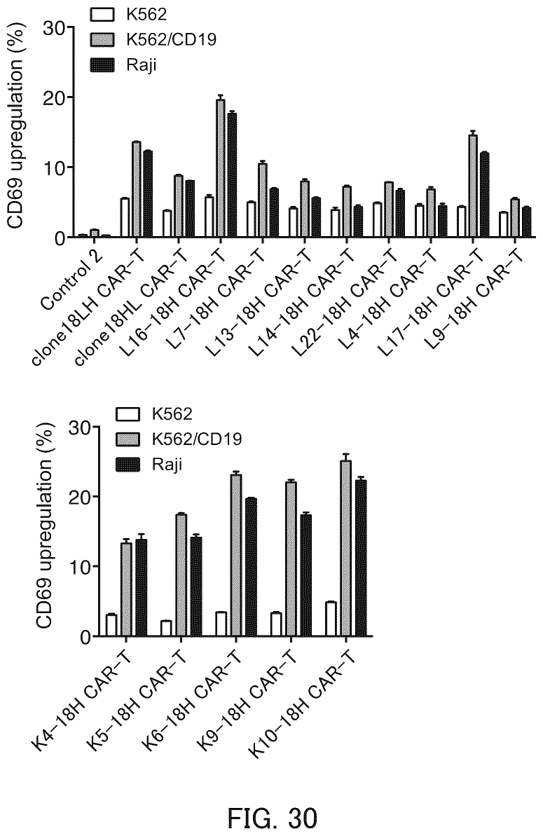

Next, it was examined if the CAR-Ts obtained in (6) of Example 10 above bound to the target antigen and were thus activated. Specifically, the CAR-Ts and antigen-presenting cells (APCs-CD19 or Raji cells) were seeded in each well (96-well plate) such that the number of the CAR-Ts was 2×10⁵ and the number of the antigen-presenting cells (APCs-CD19 or Raji cells) was 5×10⁴, and then they were cultured together for 5 hours. The antigen-presenting cells (APCs-CD19) were prepared in the same manner as in (4) of Example 10 above. An RPMI1640 culture medium containing FCS at a concentration of 10% was used as the culture solution for the coculture. After the above-mentioned coculture, the CAR-Ts were collected, and were then stained with an FITC-labeled anti-human CD69 mAb (clone: FN50) and a V450-labeled anti-human NGFR mAb. The average fluorescence intensity of CD69 in NGFR⁺ cells from the stained CAR-Ts was measured using the flow cytometer. A positive control was performed in the same manner, except that the clone18LH CAR-Ts and the clone18HL CAR-Ts obtained in (6) of Example 10 above were used. A control 1 was performed in the same manner, except that K562 cells into which the nucleic acid coding for CD19 had not been introduced were used. A control 2 was performed in the same manner, except that the expression vectors that included the polynucleotides coding for the CARs were not introduced. Then, the rates of an increase in CD69 expression in the samples were calculated using the average fluorescence intensity of CD69 before the coculture as a standard. FIG. 30 shows the results.

FIG. 30 shows graphs indicating changes in a CD69 expression level. In FIG. 30, the horizontal axes indicate the types of samples, and the vertical axes indicate the rate of an increase in CD69 expression. As shown in FIG. 30, the CD69 expression hardly increases in the control 2. On the other hand, when the clon18LH CAR-Ts and the clon18HL CAR-Ts (positive control) were stimulated with APCs-CD19 (K562/CD19) or Raji cells (Raji), it could be confirmed that the CD69 expression increased compared with the case of the stimulation with the control 1 (K562), and the CAR-Ts bound to CD19 and were activated. Moreover, when the L4-18H CAR-Ts, L7-18H CAR-Ts, L9-18H CAR-Ts, L13-18H CAR-Ts, L14-18H CAR-Ts, L16-18H CAR-Ts, L17-18H CAR-Ts, L22-18H CAR-Ts, K4-18H CAR-Ts, K5-18H CAR-Ts, K6-18H CAR-Ts, K9-18H CAR-Ts, or K10-18H CAR-Ts were stimulated with APCs-CD19 (K562/CD19) or Raji cells (Raji), the CD69 expression increased compared with the case of the stimulation with the control 1 (K562), and the CAR-Ts bound to CD19 and were activated. Furthermore, the rates of an increase in CD69 expression in the CAR-Ts were significantly different, and it was thus found that scFvs different in specificity for CD19 could be screened.

(8) Avidity of First Candidate scFvs

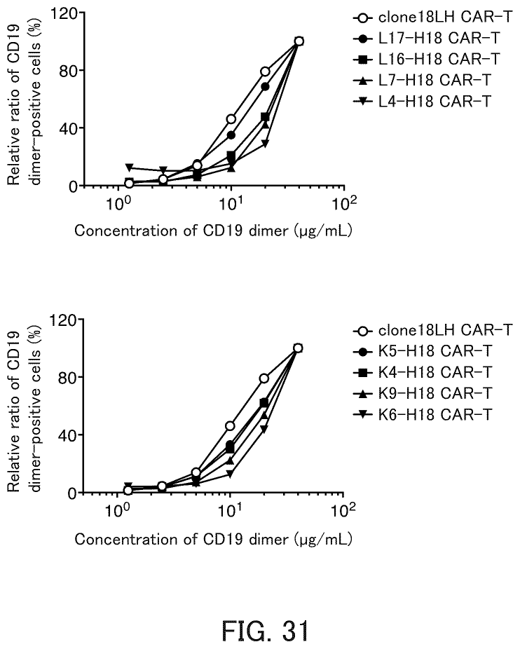

Next, it was examined if the L4-18H CAR-Ts, L7-18H CAR-Ts, L16-18H CAR-Ts, L17-18H CAR-Ts, K4-18H CAR-Ts, K5-18H CAR-Ts, K6-18H CAR-Ts, and K9-18H CAR-Ts, which were obtained in (6) of Example 10 above, were different from the clone18LH CAR-Ts in avidity for the target antigen. Flow cytometry analysis was performed on the CAR-Ts in the same manner as in (6) of Example 10 above, except that the CAR Ts were stained with the PE-labeled CD19 dimer at a predetermined concentration (1.25, 2.5, 5, 10, 20, or 40 μg/mL), and were then stained with the V450-labeled anti-human NGFR mAb. Then, after the ratios of CD19 dimer-positive cells were calculated for each CD19 dimer concentration, the relative ratios of CD19 dimer-positive cells for each CD19 dimer concentration were calculated using the ratio of CD19 dimer-positive cells for a CD19 dimer concentration of 40 μg/mL as a standard. FIG. 31 shows the results.

FIG. 31 shows graphs indicating the relative ratios of CD19 dimer-positive cells. In FIG. 31, the horizontal axes indicate the concentration of the CD19 dimer, and the vertical axes indicate the relative ratio of CD19 dimer-positive cells. As shown in FIG. 31, the ratios of the CD19 dimer-positive cells in the CAR-Ts increased in a manner dependent on the concentration of the CD19 dimer. The staining intensity varied in the CAR-Ts, and it was thus found that the scFvs in these CAR-Ts were different in the avidity for the CD19 dimer. That is, it was found that the first screening method of the present invention could be used to screen scFvs different in binding avidity for a target antigen.

(9) Responsiveness of First Candidate scFvs

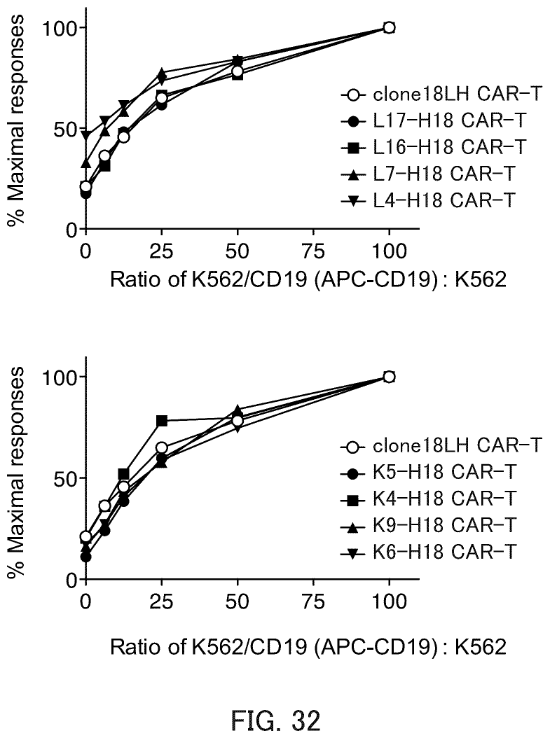

Next, it was examined if the L4-18H CAR-Ts, L7-18H CAR-Ts, L16-18H CAR-Ts, L17-18H CAR-Ts, K4-18H CAR-Ts, K5-18H CAR-Ts, K6-18H CAR-Ts, and K9-18H CAR-Ts, which were obtained in (6) above, were different from the clone18LH CAR-Ts in responsiveness to cells expressing the target antigen. Specifically, the CAR-Ts and mixed cells of the antigen-presenting cells (APCs-CD19) and K562 cells were seeded in each well (96-well plate) such that the number of the CAR-Ts was 2×10⁵ and the number of the mixed cells was 5×10⁴, and then they were cultured together for 5 hours. The ratio between the APCs-CD19 (A) and the K562 (K) in the mixed cells (A:K) was set to a predetermined ratio (0:100, 6.25:93.75, 12.5:87.5, 50:50, 100:0). After the above-mentioned coculture, the CAR-Ts were collected and were then stained with an FITC-labeled anti-human CD69 mAb (clone: FN50) and a V450-labeled anti-human NGFR mAb. The ratios of CD69-positive cells in NGFR⁺ cells from the stained CAR-Ts were measured using the flow cytometer. The relative ratio (% Maximal responses) of CD69-positive cells for each ratio (A:K) was calculated using the ratio of CD69-positive cells for a ratio (A:K) of 100:0 as a standard. FIG. 32 shows the results.

FIG. 32 shows graphs indicating the relative ratios of CD69-positive cells. In FIG. 32, the horizontal axes indicate the ratio (A:K), and the vertical axes indicate the relative ratio (% Maximal responses) of CD69-positive cells. As shown in FIG. 32, the ratios of the CD69-positive cells in the CAR-Ts increased in proportion to the ratio of the APCs-CD19 (A). The reactivity to the APCs-CD19 varied in the CAR-Ts, and it was thus found that the scFvs in these CAR-Ts were different in the avidity for the target antigen. That is, it was found that the first screening method of the present invention could be used to screen scFvs different in binding avidity for a target antigen.

(10) Functions of First Candidate scFvs

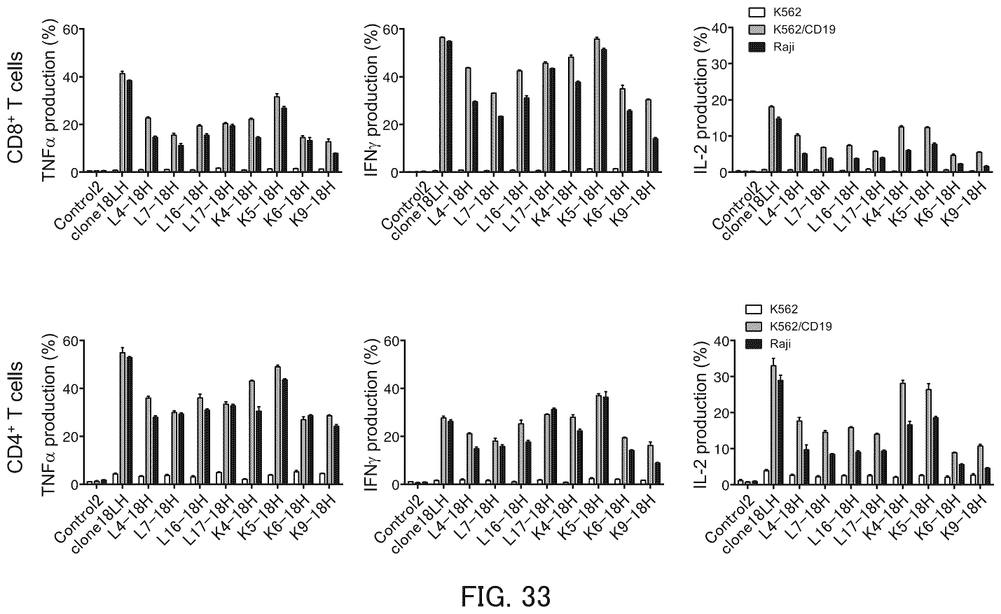

Next, it was examined if the L4-18H CAR-Ts, L7-18H CAR-Ts, L16-18H CAR-Ts, L17-18H CAR-Ts, K4-18H CAR-Ts, K5-18H CAR-Ts, K6-18H CAR-Ts, and K9-18H CAR-Ts, which were obtained in (6) above, and the clone18LH CAR-Ts bound to the target antigen and produced cytokines. Specifically, the CAR-Ts and antigen-presenting cells (APCs-CD19 or Raji cells) were seeded in each well (96-well plate) such that the number of the CAR-Ts was 3×10⁵ and the number of the antigen-presenting cells (APCs-CD19 or Raji cells) was 5×10⁴, and then they were cultured together for 5 hours. The antigen-presenting cells (APCs-CD19) were prepared in the same manner as in (4) of Example 10 above. An RPMI1640 culture medium containing FCS at a concentration of 10% was used as the culture solution for the coculture. After the above-mentioned culture, BFA was added to a concentration of 5 μmol/L, and then the culture was continued for another 5 hours. Then, after the culture, the cells were fixed with 1% PFA and subjected to cell membrane permeabilization. Moreover, the cells were stained with an APC-labeled anti-human IL-2 mAb (clone: MQ1-17H12), a PE-labeled anti-human TNFα mAb (clone: Mab11), a PC7-labeled anti-human IFN-γ mAb (clone: B27), an FITC-labeled anti-human CD4 mAb (clone: OKT4), a PC5-labeled anti-human CD8a mAb (clone: B9.11), and a V450-labeled anti-human NGFR mAb (clone: C40-1457). The stained cells were measured using the flow cytometer. Then, the ratios of cytokine expression were calculated. A positive control was performed in the same manner, except that the clone18LH CAR-Ts obtained in (6) of Example 10 above were used. A control 1 was performed in the same manner, except that K562 cells into which the nucleic acid coding for CD19 had not been introduced were used. A control 2 was performed in the same manner, except that the expression vectors that included the polynucleotides coding for the CARs were not introduced. FIG. 33 shows the results.

FIG. 33 shows graphs indicating the ratios of cytokine-producing cells. In FIG. 33, the horizontal axes indicate the types of CAR-Ts, and the vertical axes indicate the ratio of cytokine-producing cells. In FIG. 33, the upper graphs show the results from CD8-positive CAR-Ts, and the lower graphs show the results from CD4-positive CAR-Ts. As shown in FIG. 33, it was found that, in the presence of the APCs-CD19 expressing CD19 serving as a target antigen or Raji cells, the ratios of TNF-α-producing cells, IFN-γ-producing cells, and IL-2-producing cells in the CAR-Ts increased compared with the control 1 (K562) and the control 2, which did not express the target antigen, and the CAR-Ts were activated. It was found from these results that the screening method of the present invention could be used to screen scFvs capable of binding to CD19 and inducing an effector function in CAR-Ts.

### Example 11

It was confirmed that CARs that included scFvs obtained using the screening method of the present invention in which the CAR library of the present invention is used exhibited anti-tumor effects in vivo.

The L17-18H CAR-Ts, the K5-18H CAR-Ts, and the clone18LH CAR-Ts were prepared in the same manner as in Example 10 above.

Raji cells expressing SLR (Raji/SLR cells) were prepared in the same manner as in (4) of Example 1 above, except that Raji cells were used instead of K562 cells, and a polynucleotide coding for SLR was used instead of the polynucleotide coding for human CD19. It should be noted that, since SLR is an intracellular protein, a nucleic acid coding for a truncated NGFR gene (ΔNGFR) was linked to the polynucleotide coding for SLR via a polynucleotide coding for the furin cleavage site (RAKR: Sequence ID No. 172), the spacer sequence (SGSG: Sequence ID No. 173), and the codon-optimized P2A sequence (ATNFSLLKQAGDVEENPGP: Sequence ID No. 174).

SLR Protein
(SLR/furin-sgsg-p2a/dNGFR, Sequence ID No. 311)
MEEENIVNGDRPRDLVFPGTAGLQLYQSLYKYSYITDGIIDAHTNEVI
SYAQIFETSCRLAVSLEKYGLDHNNVVAICSENNIHFFGPLIAALYQG
IPMATSNDMYTEREMIGHLNISKPCLMFCSKKSLPFILKVQKHLDFLK
KVIVIDSMYDINGVECVFSFVSRYTDHAFDPVKFNPKEFDPLERTALI
MTSSGTTGLPKGVVISHRSITIRFVHSSDPIYGTRIAPDTSILAIAPF
HHAFGLFTALAYFPVGLKIVMVKKFEGEFFLKTIQNYKIASIVVPPPI
MVYLAKSPLVDEYNLSSLTEIACGGSPLGRDIADKVAKRLKVHGILQG
YGLTETCSALILSPNDRELKKGAIGTPMPYVQVKVIDINTGKALGPRE
KGEICFKSQMLMKGYHNNPQATRDALDKDGWLHTGDLGYYDEDRFIYV
VDRLKELIKYKGYQVAPAELENLLLQHPNISDAGVIGIPDEFAGQLPS
ACVVLEPGKTMTEKEVQDYIAELVTTTKHLRGGVVFIDSIPKGPTGKL
MRNELRAIFAREQAKSKLRAKRSGSGATNFSLLKQAGDVEENPGPMDG
PRLLLLLLLGVSLGGAKEACPTGLYTHSGECCKACNLGEGVAQPCGAN
QTVCEPCLDSVTFSDVVSATEPCKPCTECVGLQSMSAPCVEADDAVCR
CAYGYYQDETTGRCEACRVCEAGSGLVFSCQDKQNTVCEECPDGTYSD
EANHVDPCLPCTVCEDTERQLRECTRWADAECEEIPGRWITRSTPPEG
SDSTAPSTQEPEAPPEQDLIASTVAGVVTTVMGSSQPVVTRGTTDNLI
PVYCSILAAVVVGLVAYIAFKRWNS
Polynucleotide Coding for SLR
(Sequence ID No. 312)
5′-ATGGAAGAAGAGAACATCGTGAATGGCGATCGCCCTCGGGATCTG
GTGTTCCCTGGCACAGCCGGCCTGCAGCTGTATCAGTCCCTGTATAAA
TACTCTTACATCACCGACGGAATCATCGACGCCCACACCAACGAGGTG
ATCTCCTATGCCCAGATTTTCGAAACAAGTTGCCGCCTGGCCGTGAGC
CTGGAGAAGTATGGCCTGGATCACAACAACGTGGTGGCCATTTGCAGC
GAGAACAACATCCACTTCTTCGGCCCTCTGATCGCTGCCCTATACCAG
GGGATTCCAATGGCCACATCCAACGATATGTACACCGAGAGGGAGATG
ATCGGCCACCTGAACATCTCCAAGCCATGTCTGATGTTCTGTTCCAAG
AAGTCCCTGCCATTCATCCTGAAGGTGCAGAAGCACCTGGACTTTCTC
AAGAAGGTGATCGTGATCGACAGCATGTACGACATCAACGGCGTGGAG
TGCGTGTTCAGTTTCGTGTCCCGGTACACCGATCATGCGTTCGATCCA
GTGAAGTTCAACCCTAAAGAGTTTGATCCCCTGGAGAGAACCGCGCTG
ATCATGACATCCTCTGGAACAACCGGCCTGCCTAAGGGCGTGGTGATC
AGCCACAGGAGCATCACCATCAGATTCGTCCACAGCAGCGATCCCATC
TACGGCACCCGCATCGCCCCAGATACATCCATCCTGGCCATCGCCCCT
TTCCACCACGCCTTCGGACTGTTTACCGCCCTGGCTTACTTTCCAGTG
GGCCTGAAGATCGTGATGGTGAAAAAGTTTGAGGGCGAGTTCTTCCTG
AAGACCATCCAGAACTACAAGATCGCTTCTATCGTGGTGCCTCCTCCA
ATCATGGTGTATCTGGCCAAGAGCCCTCTGGTGGATGAGTACAATCTG
TCCAGCCTGACAGAGATCGCCTGTGGCGGCTCCCCTCTGGGCAGAGAC
ATCGCCGACAAGGTGGCCAAGAGACTGAAGGTCCACGGCATCCTGCAG
GGCTATGGCCTGACCGAGACCTGTAGCGCCCTGATCCTGAGCCCCAAC
GATAGAGAGCTGAAGAAGGGCGCCATCGGCACCCCTATGCCCTATGTC
CAGGTGAAGGTGATTGACATCAACACCGGCAAAGCCCTGGGACCAAGA
GAGAAGGGCGAGATTTGCTTCAAGAGCCAGATGCTGATGAAGGGCTAC
CACAACAACCCACAGGCCACCAGGGATGCCCTGGACAAGGACGGGTGG
CTGCACACCGGCGATCTGGGCTACTACGACGAGGACAGATTCATCTAT
GTGGTGGATCGGCTGAAAGAACTCATCAAGTACAAGGGCTACCAGGTG
GCCCCTGCCGAGCTGGAGAACTTGCTTCTGCAGCACCCTAACATCTCT
GATGCCGGCGTCATCGGCATCCCAGACGAGTTTGCCGGCCAGCTGCCT
TCCGCCTGTGTCGTGCTGGAGCCTGGCAAGACCATGACCGAGAAGGAG
GTGCAGGATTATATCGCCGAGCTGGTGACCACCACCAAGCACCTGCGG
GGCGGCGTGGTGTTCATCGACAGCATTCCGAAAGGCCCAACAGGCAAG
CTGATGAGAAACGAGCTGAGGGCCATCTTTGCCCGCGAGCAGGCCAAG
TCCAAGCTGAGGGCCAAGCGGTCCGGATCCGGAGCCACCAACTTCAGC
CTGCTGAAGCAGGCCGGCGACGTGGAGGAGAACCCCGGCCCCATGGAC
GGGCCGCGCCTGCTGCTGTTGCTGCTTCTGGGGGTGTCCCTTGGAGGT
GCCAAGGAGGCATGCCCCACAGGCCTGTACACACACAGCGGTGAGTGC
TGCAAAGCCTGCAACCTGGGCGAGGGTGTGGCCCAGCCTTGTGGAGCC
AACCAGACCGTGTGTGAGCCCTGCCTGGACAGCGTGACGTTCTCCGAC
GTGGTGAGCGCGACCGAGCCGTGCAAGCCGTGCACCGAGTGCGTGGGG
CTCCAGAGCATGTCGGCGCCATGCGTGGAGGCCGACGACGCCGTGTGC
CGCTGCGCCTACGGCTACTACCAGGATGAGACGACTGGGCGCTGCGAG
GCGTGCCGCGTGTGCGAGGCGGGCTCGGGCCTCGTGTTCTCCTGCCAG
GACAAGCAGAACACCGTGTGCGAGGAGTGCCCCGACGGCACGTATTCC
GACGAGGCCAACCACGTGGACCCGTGCCTGCCCTGCACCGTGTGCGAG
GACACCGAGCGCCAGCTCCGCGAGTGCACACGCTGGGCCGACGCCGAG
TGCGAGGAGATCCCTGGCCGTTGGATTACACGGTCCACACCCCCAGAG
GGCTCGGACAGCACAGCCCCCAGCACCCAGGAGCCTGAGGCACCTCCA
GAACAAGACCTCATAGCCAGCACGGTGGCAGGTGTGGTGACCACAGTG
ATGGGCAGCTCCCAGCCCGTGGTGACCCGAGGCACCACCGACAACCTC
ATCCCTGTCTATTGCTCCATCCTGGCTGCTGTGGTTGTGGGTCTTGTG
GCCTACATAGCCTTCAAGAGGTGGAACAGCTGA-3′

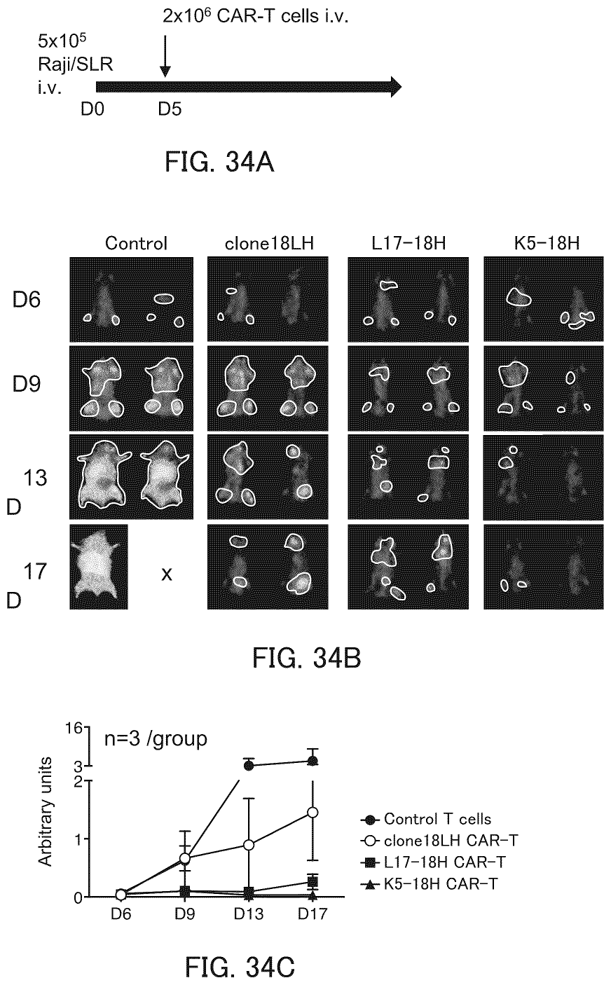

5-week-old NOG mice (NOD/Shi-scid, IL-2RγKO·Jic mice, purchased from In-Vivo Science) were irradiated with 1.5 Gy of γ rays. Next, as shown in FIG. 34(A), the Raji/SLR cells (5×10⁵ cells) were transplanted into the NOG mice through intravenous administration. The day on which the above-mentioned transplantation was performed was taken as Day 0, the CAR-Ts (2×10⁶ cells) were intravenously administered on Day 5. Then, the size of tumor was measured using an image analyzer (AEQUORIA-2D/8600 bioluminescence imaging assays, manufactured by Hamamatsu Photonics) on Days 6, 9, 13, and 17 after the transplantation. A control was measured in the same manner, except that T cells into which the CARs had not been introduced were administered. It should be noted that each group included three mice. FIG. 34(A)-(C) show the results.

FIG. 34(A)-(C) show diagrams relating to anti-tumor activity in living organisms. FIG. 34(A) indicates the protocol, FIG. 34(B) shows photographs subjected to measurement using an image analyzer, and FIG. 34(C) shows a graph indicating the sizes of tumors. In FIG. 34(B), the regions surrounded by white lines are regions in which a tumor exists. As shown in FIGS. 34(B) and 34(C), the sizes of tumors of the CAR-T administered groups decreased compared with the control. Moreover, the sizes of the tumors of the L17-18H CAR-T administered group or K5-18H CAR-T administered group significantly decreased compared with the clone18LH CAR-T administered group. It was found from these results that the CARs that included scFvs obtained using the screening method of the present invention in which the CAR library of the present invention is used exhibited anti-tumor effects in vivo. Also, it was found that the screening method of the present invention could be used to screen scFvs capable of inducing a better effector function in vivo compared with scFvs used as templates.

### Example 12

It was confirmed that CARs that included scFvs that can functionally activate CAR-T cells could be screened by performing selection based on an indirect evaluation method in the first selection step and the second selection step of the screening method of the present invention in which the CAR library of the present invention is used, that is, by performing selection based on evaluation of activation indices of candidate immune cells resulting from binding between a target antigen and a CAR.

The results in (5) and (7) of Example 2 were used to calculate, as a structural avidity index value, the tetramer concentration (EC50) at which the ratio of the tetramer positive cells in the L1-3M4E5 CAR-Ts, L66-3M4E5 CAR-Ts, L73-3M4E5 CAR-Ts, L88-3M4E5 CAR-Ts, L102-3M4E5 CAR-Ts, L124-3M4E5 CAR-Ts, H73-3M4E5 CAR-Ts, H1-3M4E5 CAR-Ts, or 3M4E5LH CAR-Ts was half as high as the maximum ratio of the positive cells. The structural avidity can also be considered as specificity of a CAR (scFv) for a target antigen. Moreover, the results in (6) and (8) of Example 2 were used to calculate, as a functional avidity index value, the tetramer concentration (EC) at which the rate of an increase in CD69 expression in the L1-3M4E5 CAR-Ts, L66-3M4E5 CAR-Ts, L73-3M4E5 CAR-Ts, L88-3M4E5 CAR-Ts, L102-3M4E5 CAR-Ts, L124-3M4E5 CAR-Ts, H73-3M4E5 CAR-Ts, H1-3M4E5 CAR-Ts, or 3M4E5LH CAR-Ts was half as high as the maximum rate of an increase in CD69 expression. The functional avidity refers to an ability of a CAR to activate T cells when the CAR binds to a target antigen. Then, the correlation between the structural avidity index value and the functional avidity index value of the L1-3M4E5 CAR-Ts, L66-3M4E5 CAR-Ts, L73-3M4E5 CAR-Ts, L88-3M4E5 CAR-Ts, L102-3M4E5 CAR-Ts, L124-3M4E5 CAR-Ts, H73-3M4E5 CAR-Ts, H1-3M4E5 CAR-Ts, or 3M4E5LH CAR-Ts was examined. FIG. 35 shows the results.

FIG. 35 is a graph indicating a relationship between the structural avidity index value and the functional avidity index value. In FIG. 35, the horizontal axis indicates the structural avidity index value (Structural avidity), and the vertical axis indicates the functional avidity index value (Functional avidity). As shown in FIG. 35, a positive correlation is observed in a region in which the structural avidity index value and the functional avidity index value are high, whereas a correlation between the structural avidity index value and the functional avidity index value is not observed as the structural avidity index value decreases. It was found from these results that CARs that included scFvs capable of functionally activating CAR-T cells could be screened by selecting candidate scFvs based on evaluation of activation indices of candidate immune cells resulting from binding between a target antigen and a CAR compared with the case where candidate scFvs are selected based on binding between a target antigen and a CAR.

### Example 13

It was confirmed that CARs that included scFvs obtained using the screening method of the present invention in which the CAR library of the present invention is used exhibited cytotoxic activity in vitro.

The L17-18H CAR-Ts, the K5-18H CAR-Ts, and the clone18LH CAR-Ts were prepared in the same manner as in Example 10 above.

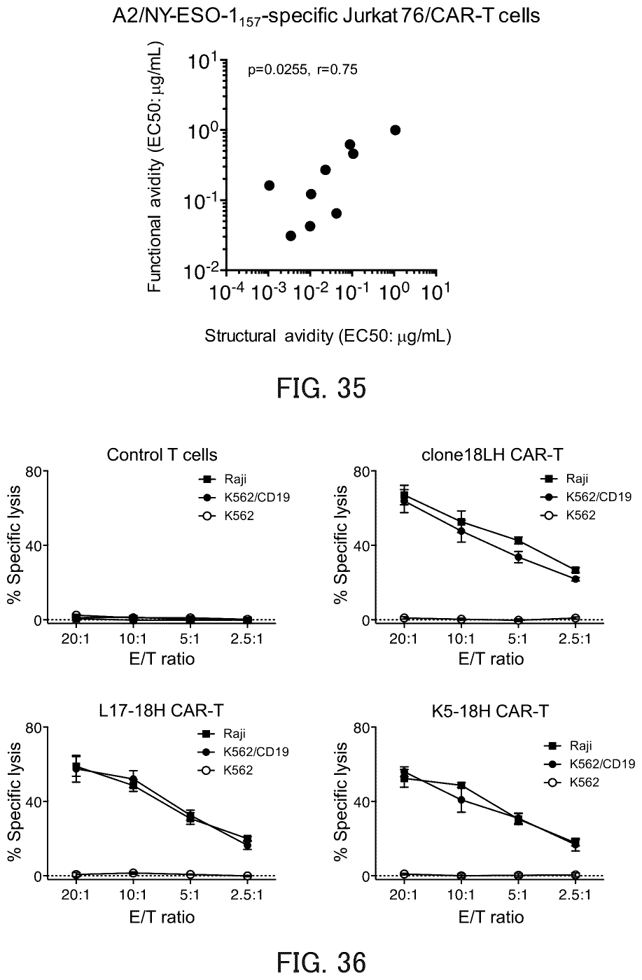

Next, 5.0×10³ APCs-CD19 (K562/CD19) or Raji cells (Raji) serving as target cells were cultured in the presence of chromium 51 (⁵¹Cr) for 1.5 hours and were thus labeled with chromium 51. Then, the L17-18H CAR-Ts, K5-18H CAR-Ts, or clone18LH CAR-Ts (E) were added to the well in which the chromium 51-labeled target cells (T) were cultured such that a ratio E:T was 20:1, 10:1, 5:1, or 2.5:1. After the cells were cultured for 5 to 6 hours, the supernatant was collected, and the radiation counting rate per minute (CPM: count per minute) was measured using AccuFLEXg7010 (manufactured by HITACHI). A control 1 was measured in the same manner, except that T cells into which NGFR expression vectors had been introduced were used instead of the CAR-T cells. A control 2 was measured in the same manner, except that K562 cells were used as the target cells. Then, cytotoxicity (ratio of specific lysis) was measured based on Formula (1) below. FIG. 36 shows the results.

$$K = ( {L}_{E} - {L}_{S} ) / ( {L}_{Max} - {L}_{S} ) \times 100 ( \% ) \qquad ( 1 )$$

K: cytotoxicity
L_{E}: CPM measurement value
L_{S}: CPM measurement value obtained when only target cells were present
L_{Max}: CPM measurement value obtained when PBS containing 0.2% Triton-X was added to well containing only target cells

FIG. 36 shows graphs indicating cytotoxicity. In FIG. 36, the horizontal axes indicate the E/T ratios, and the vertical axes indicate cytotoxicity. As shown in FIG. 36, when K562 expressing no target antigen CD19 was used as the target cells (control 2), specific cell damage did not occur, and the equal cytotoxicity was exhibited for all the groups. Moreover, when T cells that did not express CARs specific to CD19 were used (control 1), specific cell damage occurred in none of the target cells. On the other hand, when K562 and Raji cells expressing the target antigen CD19 were used as the target cells, cytotoxicity increased in a manner dependent on the effector level in the L17-18H CAR-T addition group, K5-18H CAR-T addition group, or clone18LH CAR-T addition group. The cytotoxicity was not significantly different between the L17-18H CAR-T addition group, the K5-18H CAR-T addition group, and the clone18LH CAR-T addition group. It was found from these results that the CARs that included the scFvs obtained using the screening method of the present invention in which the CAR library of the present invention is used exhibited cytotoxic activity in vitro, and the cytotoxic activity of the L17-18H CAR-Ts and the K5-18H CAR-Ts was equal to that of the clone18LH CAR-Ts.

### Example 14

It was confirmed that CARs that included scFvs obtained using the screening method of the present invention in which the CAR library of the present invention is used exhibited anti-tumor effects in vivo.

The L17-18H CAR-Ts, the K5-18H CAR-Ts, and the clone18LH CAR-Ts were prepared in the same manner as in Example 10. Then, Raji cells expressing SLR were prepared in the same manner as in Example 11.

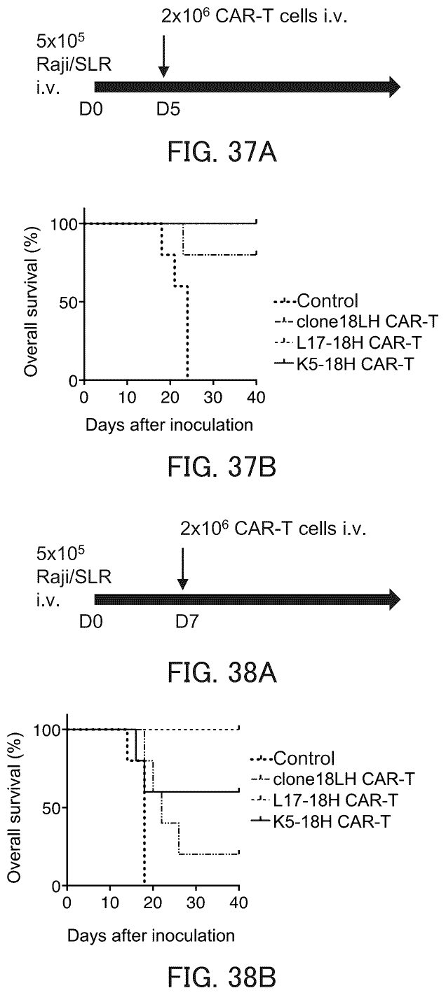

5-week-old NOG mice (NOD/Shi-scid, IL-2RγKO·Jic mice, purchased from In-Vivo Science) were radiated with 1.5 Gy of γ rays. Next, as shown in FIGS. 37(A) and 38(A), the Raji/SLR cells (5×10⁵ cells) were transplanted into the NOG mice through intravenous administration. The day on which the above-mentioned transplantation was performed was taken as Day 0, the CAR-Ts (2×10⁶ cells) were intravenously administered on Day 5 or Day 7. Then, the size of tumor was measured over time using the image analyzer after the transplantation, and the survival rate of the mice was measured. It should be noted that each group included five mice. A control was performed in the same manner, except that T cells into which NGFR expression vectors had been introduced were used instead of the CAR-T cells. FIGS. 37 and 38 show the results.

FIGS. 37 and 38 shows diagrams relating to the overall survival of living organisms. FIGS. 37(A) and 38(A) indicate protocols, and FIGS. 37(B) and 38(B) indicate the Kaplan-Meier curves showing the overall survival. As shown in FIG. 37(B), within a period during which the tumors were small, namely in an initial cancer state, the overall survival was not significantly different between the L17-18H CAR-T administered group or K5-18H CAR-T administered group and the clone18LH CAR-T administered group, and these cells exhibited substantially equal anti-tumor effects. Although not shown in the diagrams, the same results were obtained from image analysis performed using an image analyzer. On the other hand, as shown in FIG. 38(B), within a period during which sufficient infiltration of the tumors occurred, namely in an advanced tumor state, the overall survival significantly improved in the L17-18H CAR-T administered group or K5-18H CAR-T administered group compared with the clone18LH CAR-T administered group, and the overall survival was 100% in the L17-18H CAR-T administered group. Although not shown in the diagrams, the same results were obtained from image analysis performed using an image analyzer.

It was found from these results that the CARs that included the scFvs obtained using the screening method of the present invention in which the CAR library of the present invention is used exhibited anti-tumor effects in vivo. Also, it was found that the CARs that included the scFvs obtained using the screening method of the present invention in which the CAR library of the present invention is used exhibited sufficient therapeutic effects for an advanced tumor model in vivo.

### Example 15

A mechanism was examined in which scFvs having excellent in-vivo anti-tumor effects can be obtained using the screening method of the present invention.

### (1) Comparison of Cell Proliferation Capacity

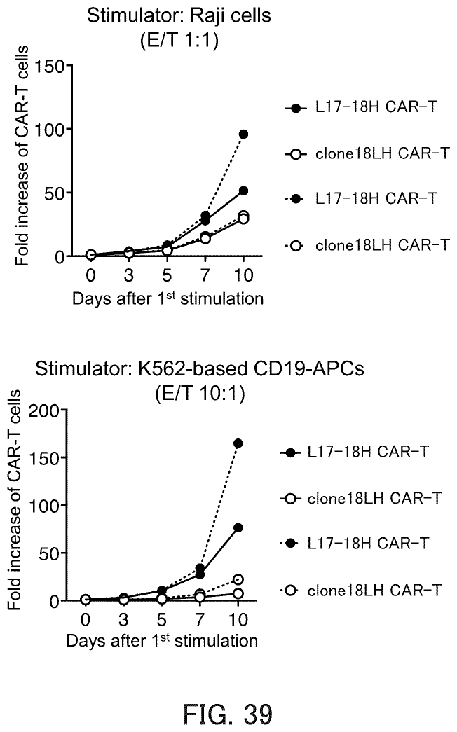

The L17-18H CAR-Ts and the clone18LH CAR-Ts were prepared in the same manner as in Example 10 above. Next, APCs-CD19 (K562/CD19) or Raji cells (Raji) (T) irradiated with 20 Gy of γ rays were added to the well in which the L17-18H CAR-Ts or clone18LH CAR-Ts (E) were cultured such that a ratio E:T was 1:1 or 10:1. The cells were cultured for 5 days after the addition of the target cells. In the above-mentioned culture, IL-2 and IL-15 were added to the culture medium such that the concentrations thereof were respectively 10 U/mL and 10 ng/mL. Furthermore, on Day 5 after the start of the culture, the CAR-T cells were restimulated in the same conditions as those at the start of the culture. Then, the number of cells at the start of the culture was taken as 1, and a ratio of the number of cells counted when a predetermined period of time (0, 3, 5, 7 or 10 days) elapsed after the start of the culture to those at the start of the culture was calculated as a proliferation rate. FIG. 39 shows the results.

FIG. 39 shows graphs indicating the cell proliferation rates. In FIG. 39, the horizontal axes indicate the period of time after the start of the culture, and the vertical axes indicate the proliferation rate. As shown in FIG. 39, at both ratios (E:T), the CARs obtained using the screening method of the present invention had a better ability to induce the proliferation of T cells. Moreover, the L17-18H CAR-Ts also exhibited a better proliferation rate in the case where E:T=1:1 compared with the clone18LH CAR-Ts, and thus it was found that the L17-18H CAR-Ts had excellent proliferating ability even in the environment in which a large amount of the target antigen was present, such as a situation in which a large number of tumor cells are present due to tumor progression.

### (2) Comparison of Cytokine Producibility

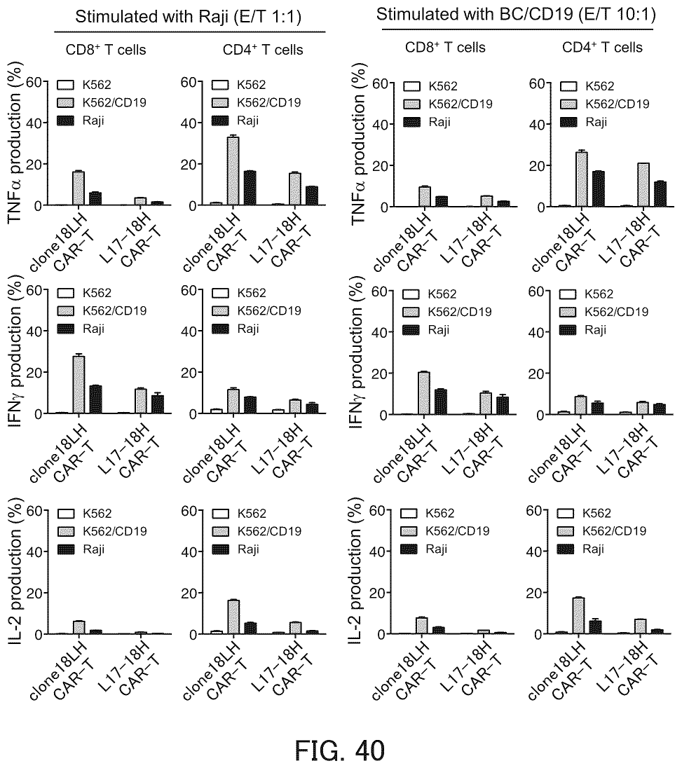

The cytokine producibility of the L17-18H CAR-Ts and the clone18LH CAR-Ts on Day 10 after the start of the culture described in (1) of Example 15 above was examined. Specifically, 3.0×10⁵ CAR-T cells and 5.0×10⁴ target cells (K562 cells, Raji cells, APCs-CD19 (K562/CD19)) were cultured together. After the cells were cultured for 2 hours, Brefeldin A (BFA) was added thereto such that the concentration thereof was 5 μg/mL, and then the cells were cultured for another 14 to 18 hours. After the CAR-T cells had been collected, the CAR-T cells were fixed with 1% paraformaldehyde (PFA), then subjected to cell membrane permeabilization, and stained with a PE-labeled anti-human TNF-α antibody (clone Mab11), an APC-labeled anti-human IL-2 antibody (clone MQ1-17H12), and a PC7-labeled anti-human IFN-γ antibody (clone B27), and a PC5-labeled anti-human CD8 antibody (clone B9.11), an FITC-labeled anti CD4 antibody (clone OKT4), and a V450-labeled anti-human NGFR (clone: C40-1457). The stained CAR-T cells were measured using the flow cytometer, and the ratios of cytokine-producing cells were calculated. FIG. 40 shows the results.

FIG. 40 shows graphs indicating the cytokine producibility. In FIG. 40, the horizontal axes indicate the types of CAR-T cells, the vertical axes indicate the ratio of cytokine-producing cells, and the culture conditions until Day 10 were shown on the upper side of the diagrams. As shown in FIG. 40, it was found that, for all types of cytokines, the ratio of the cytokine-producing cells in the L17-18H CAR-Ts was slightly lower than that in the clone18LH CAR-Ts, that is, the L17-18H CAR-Ts had slightly weak cytokine producibility.

### (3) Comparison of Cytotoxic Activity

The cytotoxic activity of the L17-18H CAR-Ts and the clone18LH CAR-Ts on Day 10 after the start of the culture described in (1) of Example 15 above was examined. Next, 5.0×10³ APCs-CD19 (K562/CD19) or Raji cells (Raji) and K562 cells serving as target cells were cultured in the presence of chromium 51 (⁵¹Cr) for 1.5 hours and were thus labeled with chromium 51.

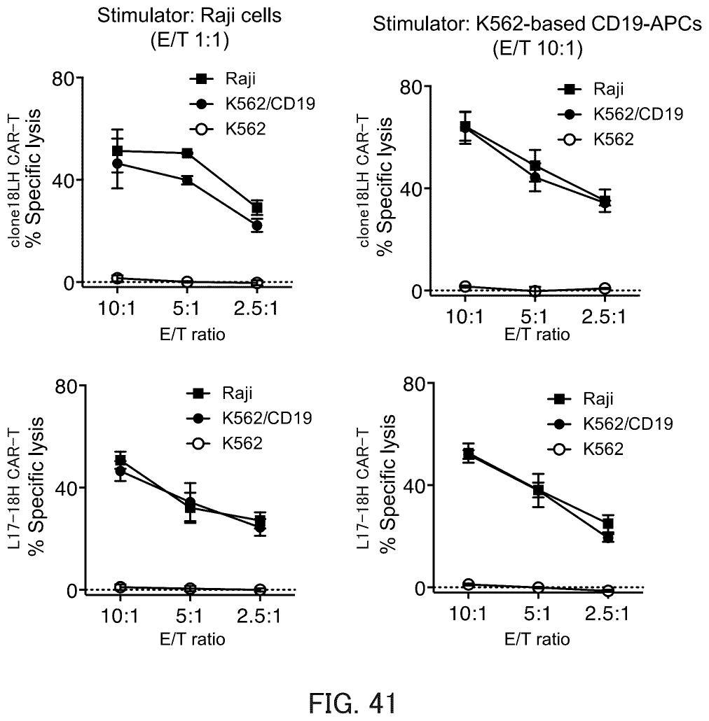

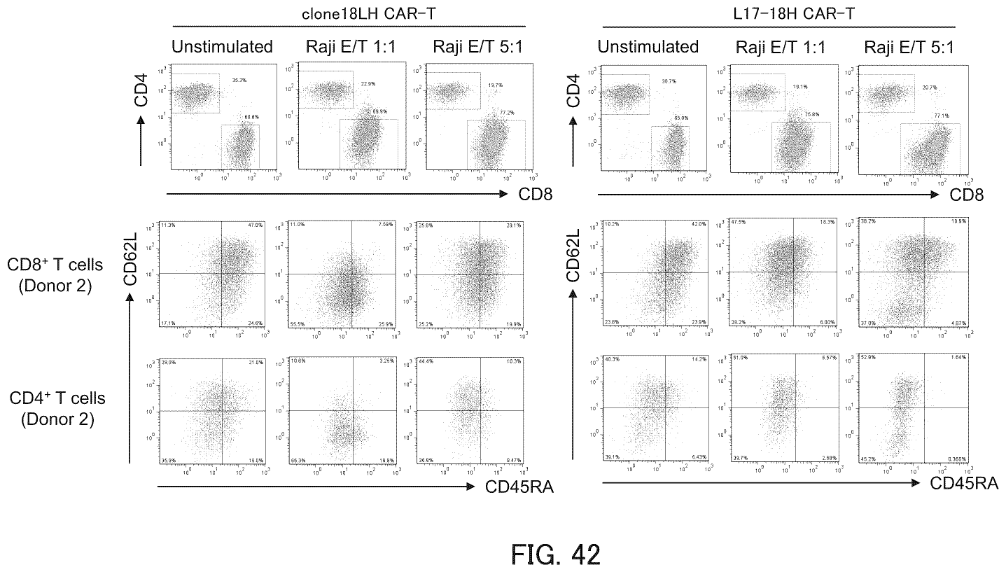

Next, the L17-18H CAR-Ts or clone18LH CAR-Ts (E) were added to the well in which the chromium 51-labeled target cells (T) were cultured such that a ratio E:T was 10:1, 5:1, or 2.5:1. Then, after the cells were cultured for 5 to 6 hours, the supernatant was collected, and the radiation counting rate per minute (CPM: count per minute) was measured using AccuFLEXg7010. Cytotoxicity (ratio of specific lysis) was measured based on Formula (1) above. FIG. 41 shows the results.

FIG. 41 shows graphs indicating cytotoxic activity. In FIG. 41, the horizontal axes indicate the ratios E:T, the vertical axes indicate cytotoxic activity, and the culture conditions until Day 10 were shown on the upper side of the diagrams. As shown in FIG. 41, it was found that the L17-18H CAR-Ts and the clone18LH CAR-Ts had equivalent cytotoxic activity.

(4) Comparison of T cell Exhaustion

The L17-18H CAR-Ts and the clone18LH CAR-Ts were prepared in the same manner as in Example 10 above, except that T cells derived from four different donors (humans) were used. The obtained CAR-T cells were incubated in PBS containing CFSE at a concentration of 0.5 μmol/L at 37° C. for 15 minutes, and were thus labeled with CFSE. Next, Raji cells (Raji) (T) irradiated with 20 Gy of γ rays and the L17-18H CAR-Ts or clone18LH CAR-Ts (E) were added to a well such that a ratio E:T was 1:1 or 5:1. The cells were cultured for 3 days after the addition of the target cells.

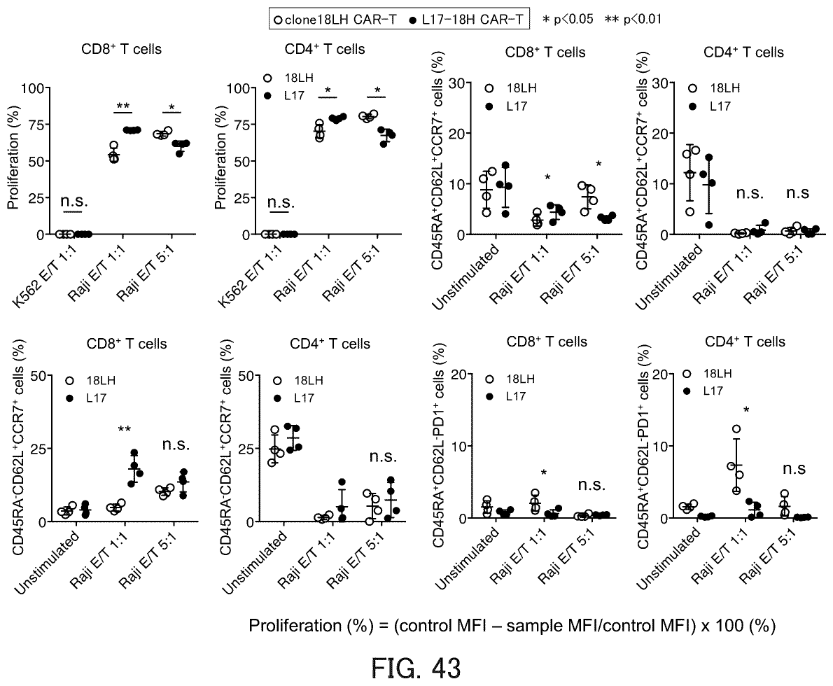

After the above-mentioned culture, the CAR-T cells were collected and stained with a PE-labeled anti-PD-1 antibody (clone EH12.2H7), an APC-labeled CD45RA antibody (clone HI100), a PC7-labeled CCR7 antibody (clone G043H7), a BV421-labeled CD62L antibody (clone DREG-56), a PC5-labeled CD8 antibody (clone B9.11), and an APC-Cy7-labeled CD4 antibody (clone RPA-T4), and then the fluorescence intensity was measured using a flow cytometer. The ratio of dividing CAR-T cells was calculated from the obtained MFI of the fluorescence intensity of CFSE based on Formula (2) below. FIG. 42 shows the distributions of CD45RA/CD62L-positive cells in CD8-positive CAR-T cells and CD4-positive CAR-T cells before and after coculture. Furthermore, FIG. 43 shows the CAR-T cell division ratios, the ratios of CD45RA-positive CD62L-positive CCR7-positive cells before and after coculture, the ratios of CD45RA-negative CD62L-positive CCR7-positive cells before and after coculture, and the ratios of CD45RA-positive CD62L-negative PD1-positive cells before and after coculture.

$$Proliferation ( \% ) = ( control MFI - sample MFI ) / control MFI \times 100 \qquad ( 2 )$$

control MFI: MFI of CAR-T cells cultured together with K562 cell strain on the condition that E:T=1:1
sample MFI: MFI of the CAR-T cells

FIG. 42 shows dot plots indicating the distributions of CD45RA/CD62L-positive cells in CD8-positive CAR-T cells and CD4-positive CAR-T cells before and after coculture. FIG. 43 shows graphs indicating the CAR-T cell division ratios, the ratios of CD45RA-positive CD62L-positive CCR7-positive cells before and after coculture, the ratios of CD45RA-negative CD62L-positive CCR7-positive cells before and after coculture, and the ratios of CD45RA-positive CD62L-negative PD1-positive cells before and after coculture. As shown in FIGS. 42 and 43, the L17-18H CAR-Ts and the clone18LH CAR-Ts had substantially the same character before the coculture. On the other hand, when stimulated in the tumor-rich environment (Raji/CAR-T E/T 1:1) that is considered to simulate the in-vivo environment, the L17-18H CAR-Ts reproducibly exhibited better proliferation capacity compared with the clone18LH CAR-Ts. Furthermore, it became clear that, in the case of the L17-18H CAR-Ts in the same environment, the CD45RA⁺CD62L⁺CCR7⁺ character of young T cells was likely to be retained, and the ratio of CD45RA⁻CD62L⁺CCR7⁺ central memory T cells increased while the ratio of exhausted CD45RA⁺CD62L⁻PD1⁺ T cells decreased, compared with the clone18LH CAR-Ts.

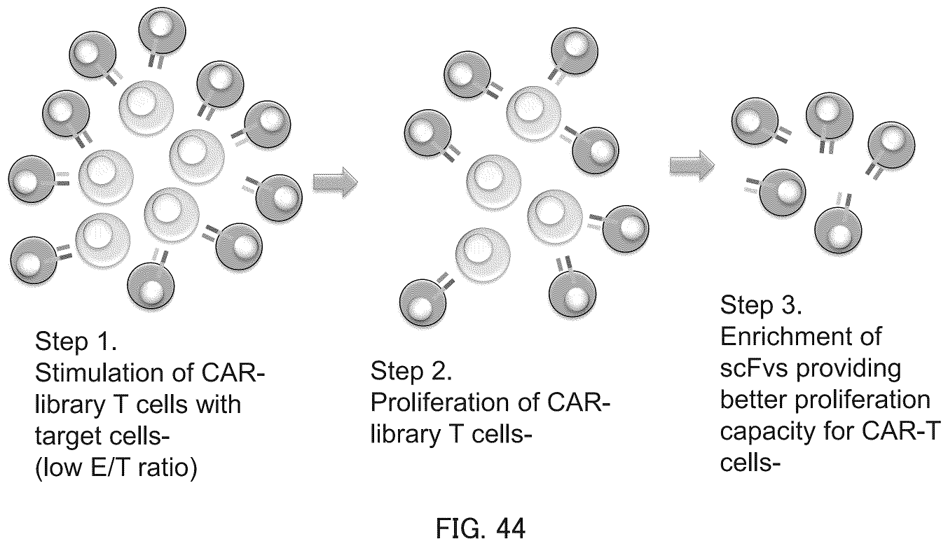

It is inferred from these results that a mechanism in which scFvs having excellent in-vivo anti-tumor effects can be obtained using the first screening method of the present invention is as follows. It should be noted that the present invention is not limited by the inference above. As shown in FIG. 44, with the screening method of the present invention, immune cells such as T cells into which a CAR library has been introduced (CAR library cells) are brought into contact with a target antigen in the first contact step or second contact step. As a result, cells expressing a CAR specific to the target antigen increase among the CAR library cells. However, each of the E/T ratios of the CAR library cells whose scFvs have different amino acid sequences is significantly low in the first contact step and the second contact step (FIG. 44 Step 1). As a result, CAR library cells characterized by being likely to proliferate even in the case of a low E/T ratio, such as the L17-18H CAR-Ts, are eventually concentrated in the first contact step and the second contact step (FIG. 44 Step 2). Then, the first selection step and the second selection step are performed in the state in which the CAR library cells having such a feature are enriched, and therefore, scFvs derived from CAR library cells characterized by being likely to proliferate even in the case of a low E/T ratio and less likely to be exhausted even when continuing to proliferate are relatively likely to be selected (FIG. 44 Step 3). On the other hand, as in the case of the L17-18H CAR-Ts, CAR library cells capable of proliferating in the case of a low E/T ratio exhibit normal cytotoxic activity. Accordingly, with the screening method of the present invention, CAR library cells that exhibit excellent proliferation capacity and include scFvs exhibiting cytotoxic activity are selected, and it is thus inferred that CARs that included scFvs that are also capable of imparting high anti-tumor activity in vivo can be screened. It should be noted that the above-mentioned mechanism is also effective even when the base skeleton of CAR is changed, and it is thus inferred that scFvs having excellent in-vivo anti-tumor effects can also be obtained even if screening is performed using CARs having intracellular domains other than those of CD28 and CD3ζ.

Although the present invention has been described with reference to the embodiments, the present invention is not limited to the embodiments described above. Various modifications that can be understood by a person skilled in the art can be made in the configurations and details of the present invention without departing from the scope of the present invention.

This application claims priority from Japanese Patent Application No. 2019-O18269 filed on Feb. 4, 2019, Japanese Patent Application No. 2019-O94188 filed on May 18, 2019, Japanese Patent Application No. 2019-140121 filed on Jul. 30, 2019, and Japanese Patent Application No. 2019-184071 filed on Oct. 4, 2019. The entire subject matter of the Japanese Patent Applications is incorporated herein by reference.

### SUPPLEMENTARY NOTES

Some or all of the above-mentioned embodiments and examples may also be described as supplementary notes below, but are not limited thereto.

### Supplementary Note 1

A chimeric antigen receptor (CAR) library comprising
nucleic acids coding for first CARS,
wherein each of the first CARs includes a first antigen-binding domain, a first transmembrane domain, and a first intracellular signaling domain,
the first antigen-binding domain includes a first single-chain antibody (scFv) to be screened for ability to bind to a target antigen,
the first scFv includes a first heavy-chain variable region and a first light-chain variable region,
the first heavy-chain variable region and the first light-chain variable region meet Condition 1 or Condition 2 below,

Condition 1 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of an antibody capable of binding to the target antigen or an antigen-binding fragment of the antibody, respectively, and
a light-chain complementarity determining region (CDRL) 1, a CDRL2, and a CDRL3 in the first light-chain variable region include a CDRL1, a CDRL2, and a CDRL3 in a light-chain variable region of a first B cell receptor, respectively, and

Condition 2 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of a first B cell receptor, respectively, and
a light-chain complementarity determining region (CDRL) 1, a CDRL2, and a CDRL3 in the first light-chain variable region include a CDRL1, a CDRL2, and a CDRL3 in a light-chain variable region of an antibody capable of binding to the target antigen or an antigen-binding fragment of the antibody, respectively.

### Supplementary Note 2

The CAR library according to Supplementary Note 1, wherein the first intracellular signaling domain includes an intracellular signaling domain of CD35.

### Supplementary Note 3

The CAR library according to Supplementary Note 1 or 2, wherein the first transmembrane domain includes a transmembrane domain of CD28.

### Supplementary Note 4

The CAR library according to any one of Supplementary Notes 1 to 3, wherein the first B cell receptor is a B cell receptor of B cells derived from a human.

### Supplementary Note 5

An scFv manufacturing method (screening method) comprising:
a first expression step of expressing the CAR library according to any one of Supplementary Notes 1 to 4 in immune cells;
a first contact step of bringing the immune cells obtained in the first expression step into contact with a target antigen; and
a first selection step of selecting a first scFvs of the CARs expressed in the immune cells that have bound to the target antigen in the first contact step as first candidate scFvs capable of binding to the target antigen.

### Supplementary Note 6

The manufacturing method (screening method) according to Supplementary Note 5, further comprising
a preparation step of preparing a second CAR library based on the first candidate scFvs,
wherein the second CAR library includes nucleic acids coding for second CARs,
each of the second CARs includes a second antigen-binding domain, a second transmembrane domain, and a second intracellular signaling domain,
the second antigen-binding domain includes a second scFv to be screened for ability to bind to the target antigen,
the second scFv includes a second heavy-chain variable region and a second light-chain variable region,
the second heavy-chain variable region and the second light-chain variable region meet Condition 3 or Condition 4 below,
the method further comprises:
   a second expression step of expressing the second CAR library in immune cells,
   a second contact step of bringing the immune cells obtained in the second expression step into contact with the target antigen; and
   a second selection step of selecting second scFvs of CARs expressed in immune cells that have bound to the target antigen in the second contact step as second candidate scFvs capable of binding to the target antigen,

Condition 3 is as follows:
if the CAR library in the first expression step meets Condition 1 above,
a CDRH1, a CDRH2, and a CDRH3 in the second heavy-chain variable region include a CDRH1, a CDRH2, and a CDRH3 in a heavy-chain variable region of a second B cell receptor, respectively, and
a CDRL1, a CDRL2, and a CDRL3 in the second light-chain variable region include a CDRL1, a CDRL2, and a CDRL3 in a light-chain variable region of the first candidate scFv, respectively, and

Condition 4 is as follows:
if the CAR library in the first expression step meets Condition 2 above,
a CDRH1, a CDRH2, and a CDRH3 in the second heavy-chain variable region include a CDRH1, a CDRH2, and a CDRH3 in a heavy-chain variable region of the first candidate scFv, respectively, and
a CDRL1, a CDRL2, and a CDRL3 in the second light-chain variable region include a CDRL1, a CDRL2, and a CDRL3 in a light-chain variable region of a second B cell receptor, respectively.

### Supplementary Note 7

The scFv manufacturing method (screening method) according to Supplementary Note 6, wherein the second contact step is performed a plurality of times.

### Supplementary Note 8

The scFv manufacturing method (screening method) according to Supplementary Note 6 or 7,
wherein immune cells capable of binding to the target antigen are detected using a monomer or multimer of the target antigen in the second selection step, and
scFvs expressed in the detected immune cells are selected as the second candidate scFvs.

### Supplementary Note 9

The scFv manufacturing method (screening method) according to any one of Supplementary Notes 6 to 8, wherein the second B cell receptor is a B cell receptor of B cells derived from a human.

### Supplementary Note 10

The scFv manufacturing method (screening method) according to any one of Supplementary Notes 5 to 9, wherein the first contact step is performed a plurality of times.

### Supplementary Note 11

The scFv manufacturing method (screening method) according to any one of Supplementary Notes 5 to 10,
wherein immune cells capable of binding to the target antigen are detected using a monomer or multimer of the target antigen in the first selection step, and
scFvs expressed in the detected immune cells are selected as the first candidate scFvs.

### Supplementary Note 12

The scFv manufacturing method (screening method) according to any one of Supplementary Notes 5 to 11, wherein the immune cells are T cells.

### Supplementary Note 13

An antibody against a complex of HLA-A*02:01 and NY-ESO-1₁₅₇₋₁₆₅ or an antigen-binding fragment of the antibody, comprising a heavy-chain variable region of (H) below and a light-chain variable region of (L) below:
(H) a heavy-chain variable region that includes
a heavy-chain complementarity determining region (CDRH) 1, a CDRH2, and a CDRH3,
wherein the CDRH1 is a polypeptide that includes an amino acid sequence of (H1),
the CDRH2 is a polypeptide that includes an amino acid sequence of (H2),
the CDRH3 is a polypeptide that includes an amino acid sequence of (H3), and
the amino acid sequences of (H1), (H2), and (H3) are as follows:
(H1) an amino acid sequence of (H1-1), (H1-2), or (H1-3) below:
   (H1-1) any one of amino acid sequences of CDRH1 shown in Table 1A above,
   (H1-2) an amino acid sequence having 80% or more identity to the amino acid sequence of (H1-1), and
   (H1-3) an amino acid sequence consisting of the amino acid sequence of (H1-1) with deletion, substitution, insertion, and/or addition of one or several amino acids,
(H2) an amino acid sequence of (H2-1), (H2-2), or (H2-3) below:
   (H2-1) any one of amino acid sequences of CDRH2 shown in Table 1A above,
   (H2-2) an amino acid sequence having 80% or more identity to the amino acid sequence of (H2-1), and
   (H2-3) an amino acid sequence consisting of the amino acid sequence of (H2-1) with deletion, substitution, insertion, and/or addition of one or several amino acids,
(H3) an amino acid sequence of (H3-1), (H3-2), or (H3-3) below:
   (H3-1) any one of amino acid sequences of CDRH3 shown in Table 1A above,
   (H3-2) an amino acid sequence having 80% or more identity to the amino acid sequence of (H3-1), and
   (H3-3) an amino acid sequence consisting of the amino acid sequence of (H3-1) with deletion, substitution, insertion, and/or addition of one or several amino acids; and
(L) a light-chain variable region that includes
a light-chain complementarity determining region (CDRL) 1, a CDRL2, and a CDRL3,
wherein the CDRL1 is a polypeptide that includes an amino acid sequence of (L1),
the CDRL2 is a polypeptide that includes an amino acid sequence of (L2),
the CDRL3 is a polypeptide that includes an amino acid sequence of (L3), and
the amino acid sequences of (L1), (L2), and (L3) are as follows:
(L1) an amino acid sequence of (L1-1), (L1-2), or (L1-3) below:
   (L1-1) any one of amino acid sequences of CDRL1 shown in Table 1B above,
   (L1-2) an amino acid sequence having 80% or more identity to the amino acid sequence of (L1-1), and
   (L1-3) an amino acid sequence consisting of the amino acid sequence of (L1-1) with deletion, substitution, insertion, and/or addition of one or several amino acids,
(L2) an amino acid sequence of (L2-1), (L2-2), or (L2-3) below:
   (L2-1) any one of amino acid sequences of CDRL2 shown in Table 1B above,
   (L2-2) an amino acid sequence having 80% or more identity to the amino acid sequence of (L2-1), and
   (L2-3) an amino acid sequence consisting of the amino acid sequence of (L2-1) with deletion, substitution, insertion, and/or addition of one or several amino acids,
(L3) an amino acid sequence of (L3-1), (L3-2), or (L3-3) below:
   (L3-1) any one of amino acid sequences of CDRL3 shown in Table 1B above,
   (L3-2) an amino acid sequence having 80% or more identity to the amino acid sequence of (L3-1), and
   (L3-3) an amino acid sequence consisting of the amino acid sequence of (L3-1) with deletion, substitution, insertion, and/or addition of one or several amino acids.

### Supplementary Note 14

The antibody or the antigen-binding fragment of the antibody according to Supplementary Note 13, wherein a combination of the heavy-chain variable region and the light-chain variable region is any one of combinations shown in Table 2 above.

### Supplementary Note 15

A chimeric antigen receptor comprising
an antigen-binding domain, a transmembrane domain, and an intracellular signaling domain,
wherein the antigen-binding domain includes the antibody or the antigen-binding fragment of the antibody according to Supplementary Note 13 or 14.

### Supplementary Note 16

The chimeric antigen receptor according to Supplementary Note 15, wherein the antigen-binding fragment is a single-chain antibody.

### Supplementary Note 17

A nucleic acid coding for the chimeric antigen receptor according to Supplementary Note 15 or 16.

### Supplementary Note 18

A cell comprising the chimeric antigen receptor according to Supplementary Note 15 or 16.

### Supplementary Note 19

The cell according to Supplementary Note 18, wherein the cell includes a T cell.

### Supplementary Note 20

A cell manufacturing method comprising an introduction step of introducing the nucleic acid according to Supplementary Note 17 into a cell.

### Supplementary Note 21

The cell manufacturing method according to Supplementary Note 20, wherein the cell includes a T cell.

### Supplementary Note 22

A bispecific antibody (BsAb) library comprising
nucleic acids coding for first BsAbs,
wherein each of the first BsAbs includes a first antigen-binding domain and a second antigen-binding domain,
the first antigen-binding domain includes a first single-chain antibody (scFv) capable of binding to a first target antigen,
the second antigen-binding domain includes a second scFv to be screened for the ability to bind to a second target antigen,
the second scFv includes a second heavy-chain variable region and a second light-chain variable region,
the second heavy-chain variable region and the second light-chain variable region meet Condition 1 or Condition 2 below,
the first target antigen or the second target antigen is an immune cell-activating receptor,

Condition 1 is as follows:
a heavy-chain complementarity determining region (CDRH) 1, a CDRH2, and a CDRH3 in the second heavy-chain variable region include a CDRH1, a CDRH2, and a CDRH3 in a heavy-chain variable region of an antibody capable of binding to the second target antigen or an antigen-binding fragment of the antibody, respectively, and
a light-chain complementarity determining region (CDRL) 1, a CDRL2, and a CDRL3 in the second light-chain variable region include a CDRL1, a CDRL2, and a CDRL3 in a light-chain variable region of a first B cell receptor, respectively, and

Condition 2 is as follows:
a heavy-chain complementarity determining region (CDRH) 1, a CDRH2, and a CDRH3 in the second heavy-chain variable region include a CDRH1, a CDRH2, and a CDRH3 in a heavy-chain variable region of a first B cell receptor, respectively, and
a light-chain complementarity determining region (CDRL) 1, a CDRL2, and a CDRL3 in the second light-chain variable region include a CDRL1, a CDRL2, and a CDRL3 in a light-chain variable region of an antibody capable of binding to the second target antigen or an antigen-binding fragment of the antibody, respectively.

### Supplementary Note 23

The BsAb library according to Supplementary Note 22, wherein the first B cell receptor is a B cell receptor of B cells derived from a human.

### Supplementary Note 24

The BsAb library according to Supplementary Note 22 or 23, wherein the immune cell-activating receptor is CD3.

### Supplementary Note 25

An scFv manufacturing method (screening method) comprising:
a first production step of producing first BsAbs from the BsAb library according to any one of Supplementary Notes 22 to 24;
a first contact step in which, when the first target antigen is an immune cell-activating receptor,
the first BsAbs, the second target antigen, and immune cells capable of expressing the immune cell-activating receptor are brought into contact with one another, and
when the second target antigen is an immune cell-activating receptor,
the first BsAbs, the first target antigen, and immune cells capable of expressing the immune cell-activating receptor are brought into contact with one another; and
a first selection step of selecting second scFvs of the first BsAbs that have bound to the second target antigen in the first contact step as first candidate scFvs capable of binding to the second target antigen.

### Supplementary Note 26

The scFv manufacturing method (screening method) according to Supplementary Note 25, further comprising
a preparation step of preparing a second BsAb library based on the first candidate scFvs,
wherein the second BsAb library includes nucleic acids coding for the second BsAbs,
each of the second BsABs includes a third antigen-binding domain and a fourth antigen-binding domain,
the third antigen-binding domain includes a third scFv capable of binding to the first target antigen,
the fourth antigen-binding domain includes a fourth scFv to be screened for the ability to bind to the second target antigen,
the fourth scFv includes a fourth heavy-chain variable region and a fourth light-chain variable region,
the fourth heavy-chain variable region and the fourth light-chain variable region meet Condition 3 or Condition 4 below,
the method further comprises:
   a second production step of producing the second BsAbs from the second BsAb library;
   a second contact step in which, when the first target antigen is the immune cell-activating receptor,
   the second BsAbs, the second target antigen, and immune cells capable of expressing the immune cell-activating receptor are brought into contact with one another, and
   when the second target antigen is the immune cell-activating receptor,
   the second BsAbs, the first target antigen, and immune cells capable of expressing the immune cell-activating receptor are brought into contact with one another; and
   a second selection step of selecting fourth scFvs of the second BsAbs that have bound to the second target antigen in the second contact step as second candidate scFvs capable of binding to the second target antigen,

Condition 3 is as follows:
if the BsAb library in the first production step meets Condition 1 above,
a CDRH1, a CDRH2, and a CDRH3 in the fourth heavy-chain variable region include a CDRH1, a CDRH2, and a CDRH3 in a heavy-chain variable region of a second B cell receptor, respectively, and
a CDRL1, a CDRL2, and a CDRL3 in the fourth light-chain variable region include a CDRL1, a CDRL2, and a CDRL3 in a light-chain variable region of the first candidate BsAb, respectively, and

Condition 4 is as follows:
if the BsAb library in the first production step meets Condition 2 above,
a CDRH1, a CDRH2, and a CDRH3 in the fourth heavy-chain variable region include a CDRH1, a CDRH2, and a CDRH3 in a heavy-chain variable region of the first candidate BsAb, respectively, and
a CDRL1, a CDRL2, and a CDRL3 in the fourth light-chain variable region include a CDRL1, a CDRL2, and a CDRL3 in a light-chain variable region of a second B cell receptor, respectively.

### Supplementary Note 27

The scFv manufacturing method (screening method) according to Supplementary Note 26, wherein the second contact step and the second selection step are performed as one set, and this set is performed a plurality of times.

### Supplementary Note 28

The scFv manufacturing method (screening method) according to Supplementary Note 26 or 27,
wherein expression of a molecule capable of activating the immune cells is detected in the second selection step, and
a second BsAbs that have induced the molecule capable of activating the immune cells are selected as the second candidate BsAbs.

### Supplementary Note 29

The scFv manufacturing method (screening method) according to any one of Supplementary Notes 25 to 28, wherein the second B cell receptor is a B cell receptor of B cells derived from a human.

### Supplementary Note 30

The scFv manufacturing method (screening method) according to any one of Supplementary Notes 25 to 29, wherein the first contact step and the first selection step are performed as one set, and this set is performed a plurality of times.

### Supplementary Note 31

The scFv manufacturing method (screening method) according to any one of Supplementary Notes 25 to 30,
wherein expression of a molecule capable of activating the immune cells is detected in the first selection step, and
the first BsAbs that have induced the molecule capable of activating the immune cells are selected as the first candidate BsAbs.

### Supplementary Note 32

The scFv manufacturing method (screening method) according to any one of Supplementary Notes 25 to 31, wherein the immune cell-activating receptor is CD3.

### Supplementary Note 33

The scFv manufacturing method (screening method) according to any one of Supplementary Notes 25 to 32, wherein the immune cells are T cells.

### Supplementary Note 34

An antibody against CD19 or an antigen-binding fragment of the antibody, comprising a heavy-chain variable region of (H) below and a light-chain variable region of (L) below:

(H) a heavy-chain variable region that includes
a heavy-chain complementarity determining region (CDRH) 1, a CDRH2, and a CDRH3,
wherein the CDRH1 is a polypeptide that includes an amino acid sequence of (H1),
the CDRH2 is a polypeptide that includes an amino acid sequence of (H2),
the CDRH3 is a polypeptide that includes an amino acid sequence of (H3), and
the amino acid sequences of (H1), (H2), and (H3) are as follows:
(H1) an amino acid sequence of (H1-1), (H1-2), or (H1-3) below:
   (H1-1) any one of amino acid sequences of CDRH1 shown in Table 3A above,
   (H1-2) an amino acid sequence having 80% or more identity to the amino acid sequence of (H1-1), and
   (H1-3) an amino acid sequence consisting of the amino acid sequence of (H1-1) with deletion, substitution, insertion, and/or addition of one or several amino acids,
(H2) an amino acid sequence of (H2-1), (H2-2), or (H2-3) below:
   (H2-1) any one of amino acid sequences of CDRH2 shown in Table 3A above,
   (H2-2) an amino acid sequence having 80% or more identity to the amino acid sequence of (H2-1), and
   (H2-3) an amino acid sequence consisting of the amino acid sequence of (H2-1) with deletion, substitution, insertion, and/or addition of one or several amino acids,
(H3) an amino acid sequence of (H3-1), (H3-2), or (H3-3) below:
   (H3-1) any one of amino acid sequences of CDRH3 shown in Table 3A above,
   (H3-2) an amino acid sequence having 80% or more identity to the amino acid sequence of (H3-1), and
   (H3-3) an amino acid sequence consisting of the amino acid sequence of (H3-1) with deletion, substitution, insertion, and/or addition of one or several amino acids; and
(L) a light-chain variable region that includes
   a light-chain complementarity determining region (CDRL) 1, a CDRL2, and a CDRL3,
   wherein the CDRL1 is a polypeptide that includes an amino acid sequence of (L1),
   the CDRL2 is a polypeptide that includes an amino acid sequence of (L2),
   the CDRL3 is a polypeptide that includes an amino acid sequence of (L3), and
   the amino acid sequences of (L1), (L2), and (L3) are as follows:
   (L1) an amino acid sequence of (L1-1), (L1-2), or (L1-3) below:
      (L1-1) any one of amino acid sequences of CDRL1 shown in Table 3B above,
      (L1-2) an amino acid sequence having 80% or more identity to the amino acid sequence of (L1-1), and
      (L1-3) an amino acid sequence consisting of the amino acid sequence of (L1-1) with deletion, substitution, insertion, and/or addition of one or several amino acids,
   (L2) an amino acid sequence of (L2-1), (L2-2), or (L2-3) below:
      (L2-1) any one of amino acid sequences of CDRL2 shown in Table 3B above,
      (L2-2) an amino acid sequence having 80% or more identity to the amino acid sequence of (L2-1), and
      (L2-3) an amino acid sequence consisting of the amino acid sequence of (L2-1) with deletion, substitution, insertion, and/or addition of one or several amino acids,
   (L3) an amino acid sequence of (L3-1), (L3-2), or (L3-3) below:
      (L3-1) any one of amino acid sequences of CDRL3 shown in Table 3B above,
      (L3-2) an amino acid sequence having 80% or more identity to the amino acid sequence of (L3-1), and
      (L3-3) an amino acid sequence consisting of the amino acid sequence of (L3-1) with deletion, substitution, insertion, and/or addition of one or several amino acids.

### Supplementary Note 35

The antibody or the antigen-binding fragment of the antibody according to Supplementary Note 34, wherein a combination of the heavy-chain variable region and the light-chain variable region is any one of combinations shown in Table 2 above.

### Supplementary Note 36

A chimeric antigen receptor comprising
an antigen-binding domain, a transmembrane domain, and an intracellular signaling domain,
wherein the antigen-binding domain includes the antibody or the antigen-binding fragment of the antibody according to Supplementary Note 34 or 35.

### Supplementary Note 37

The chimeric antigen receptor according to Supplementary Note 36, wherein the antigen-binding fragment is a single-chain antibody.

### Supplementary Note 38

A nucleic acid coding for the chimeric antigen receptor according to Supplementary Note 36 or 37.

### Supplementary Note 39

A cell comprising the chimeric antigen receptor according to Supplementary Note 36 or 37.

### Supplementary Note 40

The cell according to Supplementary Note 39, wherein the cell includes a T cell.

### Supplementary Note 41

A cell manufacturing method comprising an introduction step of introducing the nucleic acid according to Supplementary Note 38 into a cell.

### Supplementary Note 42

The cell manufacturing method according to Supplementary Note 41, wherein the cell includes a T cell.

### Supplementary Note 43

An scFv manufacturing method comprising:
a first administration step of administering cells expressing a first chimeric antigen receptor (CAR) library to an animal, and
a first collection step of collecting the cells expressing the first CAR library that accumulate in a tissue expressing a target antigen in the animal as cells expressing CARs specific to the target antigen,
wherein the first CAR library includes nucleic acids coding for first CARs,
each of the first CARs includes a first antigen-binding domain, a first transmembrane domain, and a first intracellular signaling domain,
the first antigen-binding domain includes a first single-chain antibody (scFv) to be screened for the ability to bind to the target antigen,
the first scFv includes a first heavy-chain variable region and a first light-chain variable region,
the first heavy-chain variable region and the first light-chain variable region meet Condition 1 or Condition 2 below,

Condition 1 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of an antibody capable of binding to the target antigen or an antigen-binding fragment of the antibody, respectively, and
a light-chain complementarity determining region (CDRL) 1, a CDRL2, and a CDRL3 in the first light-chain variable region include a CDRL1, a CDRL2, and a CDRL3 in a light-chain variable region of a first B cell receptor, respectively, and

Condition 2 is as follows:
a heavy-chain complementarity determining region (CDRH) 1, a CDRH2, and a CDRH3 in the first heavy-chain variable region include a CDRH1, a CDRH2, and a CDRH3 in a heavy-chain variable region of a first B cell receptor, respectively, and
a light-chain complementarity determining region (CDRL) 1, a CDRL2, and a CDRL3 in the first light-chain variable region include a CDRL1, a CDRL2, and a CDRL3 in a light-chain variable region of an antibody capable of binding to the target antigen or an antigen-binding fragment of the antibody, respectively.

### Supplementary Note 44

The manufacturing method according to Supplementary Note 43, wherein a plurality of types of cells expressing the first CAR library are administered in the first administration step.

### Supplementary Note 45

The manufacturing method according to Supplementary Note 43 or 44, comprising a first stimulation step of administering the target antigen to the animal after the first administration step.

### Supplementary Note 46

The manufacturing method according to any one of Supplementary Notes 43 to 45, comprising a first restimulation step of restimulating the cells expressing CARs specific to the target antigen with the target antigen.

### Supplementary Note 47

The manufacturing method according to any one of Supplementary Notes 43 to 46, comprising a first formation step of forming a tissue expressing a target antigen prior to the first administration step by introducing cells expressing the target antigen into the animal.

### Supplementary Note 48

The manufacturing method according to any one of Supplementary Notes 43 to 47, wherein the cells expressing the first CAR library that accumulate in the expressing tissue are collected as cells expressing CARs specific to the target antigen.

### Supplementary Note 49

The manufacturing method according to any one of Supplementary Notes 43 to 48, wherein the first B cell receptor is a B cell receptor of B cells derived from a human.

### Supplementary Note 50

The manufacturing method according to any one of Supplementary Notes 43 to 49, further comprising:
a second administration step of administering cells expressing a second CAR library to an animal, and
a second collection step of collecting the cells expressing the second CAR library that accumulate in a tissue expressing a target antigen in the animal as cells expressing CARs specific to the target antigen,
wherein the second CAR library includes nucleic acids coding for second CARs,
each of the second CARs includes a second antigen-binding domain, a second transmembrane domain, and a second intracellular signaling domain,
the second antigen-binding domain includes a second scFv to be screened for the ability to bind to the target antigen,
the second scFv includes a second heavy-chain variable region and a second light-chain variable region,
the second heavy-chain variable region and the second light-chain variable region meet Condition 3 or Condition 4 below,

Condition 3 is as follows:
if the first CAR library meets Condition 1 above,
a CDRH1, a CDRH2, and a CDRH3 in the second heavy-chain variable region include a CDRH1, a CDRH2, and a CDRH3 in a heavy-chain variable region of a second B cell receptor, respectively, and
a CDRL1, a CDRL2, and a CDRL3 in the second light-chain variable region include a CDRL1, a CDRL2, and a CDRL3 in a light-chain variable region of an scFv of the cell expressing the CAR specific to the target antigen, respectively, and

Condition 4 is as follows:
if the first CAR library meets Condition 2 above,
a CDRH1, a CDRH2, and a CDRH3 in the second heavy-chain variable region include a CDRH1, a CDRH2, and a CDRH3 in a heavy-chain variable region of an scFv of the cell expressing the CAR specific to the target antigen, respectively, and
a CDRL1, a CDRL2, and a CDRL3 in the second light-chain variable region include a CDRL1, a CDRL2, and a CDRL3 in a light-chain variable region of a second B cell receptor, respectively.

### Supplementary Note 51

The manufacturing method according to Supplementary Note 50, wherein a plurality of types of cells expressing the second CAR library are administered in the second administration step.

### Supplementary Note 52

The manufacturing method according to Supplementary Note 50 or 51, further comprising a second stimulation step of administering the target antigen to the animal after the second administration step.

### Supplementary Note 53

The manufacturing method according to any one of Supplementary Notes 50 to 52, further comprising a first restimulation step of restimulating the cells expressing CARs specific to the target antigen with the target antigen.

### Supplementary Note 54

The manufacturing method according to any one of Supplementary Notes 50 to 53, further comprising a second formation step of forming a tissue expressing a target antigen prior to the second administration step by introducing cells expressing the target antigen into the animal.

### Supplementary Note 55

The manufacturing method according to any one of Supplementary Notes 50 to 54, wherein the tissue expressing the target antigen is collected in the second collection step, and the cells expressing the second CAR library that accumulate in the tissue expressing the target antigen are collected as cells expressing CARs specific to the target antigen.

### Supplementary Note 56

The manufacturing method according to any one of Supplementary Notes 50 to 55, wherein the second B cell receptor is a B cell receptor of B cells derived from a human.

### Supplementary Note 57

The manufacturing method according to any one of Supplementary Notes 43 to 56, wherein the expressing cells are T cells.

### Supplementary Note 58

The manufacturing method according to any one of Supplementary Notes 43 to 57, wherein the target antigen is a tumor antigen.

### Supplementary Note 59

The manufacturing method according to any one of Supplementary Notes 43 to 58, wherein the animal is a non-human animal.

### Supplementary Note 60

The manufacturing method according to any one of Supplementary Notes 43 to 59, wherein the animal is an immune-suppressed animal.

### INDUSTRIAL APPLICABILITY

As described above, with the present invention, a CAR library used to screen scFvs that can be functional in CAR-T cells, and an scFv screening method in which the CAR library is used can be provided. Therefore, it can be said that the present invention is significantly useful in the pharmaceutical field and the like.

1. An antibody or antigen-binding fragment thereof against CD19, comprising:
a heavy chain variable region of (H) below and a light chain variable region of (L) below:
(H) the heavy chain variable region comprises a heavy chain complementarity determining region (CDRH) 1, CDRH2, and CDRH3, wherein
the CDRH1 is a polypeptide comprising an amino acid sequence of SEQ ID No.: 216;
the CDRH2 is a polypeptide comprising an amino acid sequence of SEQ ID No.: 217; and
the CDRH3 is a polypeptide comprising an amino acid sequence of SEQ ID No.: 218;
(L) the light chain variable region comprises a light chain complementarity determining region (CDRL) 1, CDRL2, and CDRL3, wherein
the CDRL1 is a polypeptide comprising an amino acid sequence of SEQ ID No.: 244;
the CDRL2 is a polypeptide comprising an amino acid sequence of SEQ ID No.: 245; and
the CDRL3 is a polypeptide comprising an amino acid sequence of SEQ ID No.: 246.

2. An antibody or antigen-binding fragment thereof against CD19 according to claim 1, wherein
the heavy chain variable region of (H) comprises a heavy chain variable region of (H-A) below and the light chain variable region of (L) comprises a light chain variable region of (L-G) below, wherein
(H-A) the heavy chain variable region comprises an amino acid sequence of (H-A1), (H-A2) or (H-A3) below:
(H-A1) an amino acid sequence of SEQ ID No.: 219;
(H-A2) an amino acid sequence having 90% or more identity to the amino acid sequence of SEQ ID No.: 219 and comprising CDRH1 comprising an amino acid sequence of SEQ ID No.: 216, CDRH2 comprising an amino acid sequence of SEQ ID No.: 217, and CDRH3 comprising an amino acid sequence of SEQ ID No.: 218;
(H-A3) an amino acid sequence consisting of the amino acid sequence of SEQ ID No.: 219 with deletion, substitution, insertion, and/or addition of one to ten amino acids and comprising CDRH1 comprising the amino acid sequence of SEQ ID No.: 216, CDRH2 comprising the amino acid sequence of SEQ ID No.: 217, and CDRH3 comprising the amino acid sequence of SEQ ID No.: 218;
(L-G) the light chain variable region comprises an amino acid sequence of (L-G1), (L-G2) or (L-G3) below:
(L-G1) an amino acid sequence of SEQ ID No.: 247;
(L-G2) an amino acid sequence having 90% or more identity to the amino acid sequence of SEQ ID No.: 247 and comprising CDRL1 comprising an amino acid sequence of SEQ ID No.: 244, CDRL2 comprising an amino acid sequence of SEQ ID No.: 245, and CDRL3 comprising an amino acid sequence of SEQ ID No.: 246;
(L-G3) an amino acid sequence consisting of the amino acid sequence of SEQ ID No.: 247 with deletion, substitution, insertion, and/or addition of one to ten amino acids and comprising CDRL1 comprising the amino acid sequence of SEQ ID No.: 244, CDRL2 comprising the amino acid sequence of SEQ ID No.: 245, and CDRL3 comprising the amino acid sequence of SEQ ID No.: 246.

3. A chimeric antigen receptor, comprising:
an antigen-binding domain, a transmembrane domain, and an intracellular signaling domain,
wherein the antigen-binding domain comprises the antibody or the antigen-binding fragment thereof according to claim 1.

4. The chimeric antigen receptor according to claim 3, wherein
the antigen binding fragment is a single chain antibody.

5. The chimeric antigen receptor according to claim 3, wherein
the antigen binding domain comprises a polypeptide of (bd) below:
(bd) the polypeptide of (bd1), (bd2) or (bd3) below:
(bd1) a polypeptide comprising the amino acid sequence of SEQ ID No.: 278;
(bd2) a polypeptide comprising an amino acid sequence having 90% or more identity to the amino acid sequence of SEQ ID No.: 278, the amino acid sequences corresponding to CDRH1, CDRH2, CDRH3, CDRL1, CDRL2, and CDRL3 of the amino acid sequence of SEQ ID No.: 278 being conserved, and the polypeptide being capable of binding to CD19;
(bd3) a polypeptide comprising an amino acid sequence of SEQ ID No.: 278 with deletion, substitution, insertion, and/or addition of one to twenty amino acids, the amino acid sequences corresponding to CDRH1, CDRH2, CDRH3, CDRL1, CDRL2, and CDRL3 of the amino acid sequence of SEQ ID No.: 278 being conserved, and the polypeptide being capable of binding to CD19.

6. A nucleic acid coding the antibody or antigen-binding fragment thereof according to claim 1.

7. A transformant, comprising:
a host and the nucleic acid according to claim 6.

8. A nucleic acid coding the chimeric antigen receptor according to claim 3.

9. A cell, comprising:
the chimeric antigen receptor according to claim 3.

10. The cell according to claim 9, wherein
the cell comprises a T cell.

11. A method for producing a cell, the method comprising
introducing the nucleic acid according to claim 8 into the cell.

12. The method for producing a cell according to claim 11, wherein
the cell comprises a T cell.
